(19) United States
(12) Patent Application Publication
(10) Pub. No.: US 2016/0194358 A1
(43) Pub. Date: Jul. 7, 2016

(54) LINEAR PEPTIDE ANTIBIOTCS

(71) Applicant: RQX PHARMACEUTICALS, INC., La Jolla, CA (US)

(72) Inventors: Peter Andrew SMITH, La Jolla, CA (US); Tucker Curran ROBERTS, San Diego, CA (US); Robert I. HIGUCHI, Solana Beach, CA (US); Prasuna PARASELLI, San Diego, CA (US); Michael F.T. KOEHLER, Palo Alto, CA (US); James John CRAWFORD, San Francisco, CA (US)

(21) Appl. No.: 14/911,823

(22) PCT Filed: Aug. 14, 2014
(86) PCT No.: PCT/US2014/051151

§ 371 Date: Feb. 12, 2016

Related U.S. Application Data
(60) Provisional application No. 61/865,980, filed on Aug. 14, 2013.
(60) Provisional application No. 61/865,986, filed on Aug. 14, 2013.
(60) Provisional application No. 61/865,982, filed on Aug. 14, 2013.

(51) Int. Cl. C07K 7/06

(52) U.S. Cl. CPC C07K 7/06; A61K 38/00

(57) ABSTRACT

Provided herein are antibacterial compounds, wherein the compounds in some embodiments have broad spectrum bioactivity. The compounds provided herein can in other embodiments overcome the resistance conferred by single amino acid mutations at defined positions of bacterial Signal Peptidases (SPases) and in other embodiments provide for a broad spectrum of antibiotic bioactivity. Pharmaceutical compositions and methods for treatment using the compounds described herein are also provided.

### CROSS REFERENCE

This application claims the benefit of U.S. Provisional Application Ser. No. 61/865,980, filed Aug. 14, 2013; U.S. Provisional Application Ser. No. 61/865,982, filed Aug. 14, 2013; U.S. Provisional Application Ser. No. 61/865,986, filed Aug. 14, 2013; each of which is incorporated herein by reference in their entirety.

### BACKGROUND OF THE INVENTION

Since the appearance of the first antibiotic-resistant bacterial strains in the 1940's, at least thirteen strains that are impervious to many antibiotics have been discovered. According to the Infectious Disease Society of America, bacteria that are resistant to one or more drugs are responsible for some 100,000 U.S. hospital deaths a year, and cost the health care system more than $34 billion. The discovery of new antibiotics, especially those that act via the inhibition of a novel target, is an urgent need.

### SUMMARY OF THE INVENTION

Described herein are linear peptides for the treatment of microbial infections, such as for the treatment of bacterial infections. In various embodiments, the present disclosure provides lipopeptide compounds for the treatment of bacterial infections. In various embodiments, the lipopeptide compounds act by inhibition of bacterial type 1 signal peptidase (SpsB), an essential protein in bacteria.

In one aspect described herein are compounds having the structure of Formula (A):

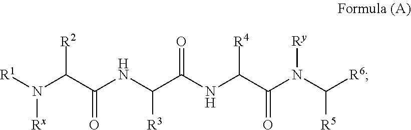

wherein:
R¹ is selected from:

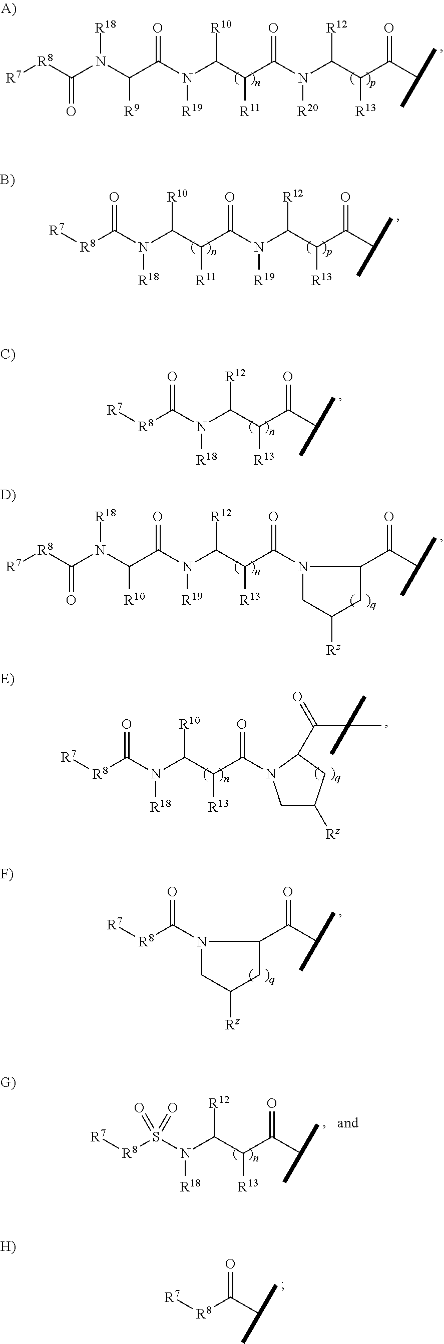

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

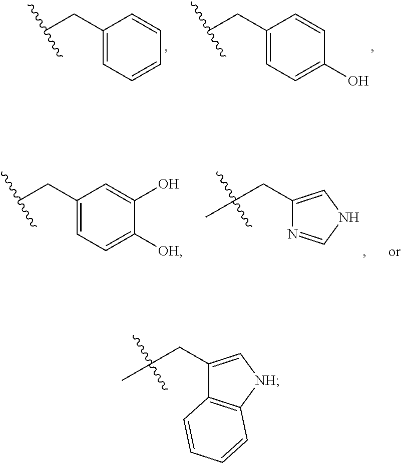 [SMILES: *Cc1ccccc1 ; *Cc1ccc(O)cc1 ; *Cc1ccc(O)c(O)c1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heterocycloalkyl, or an optionally substituted

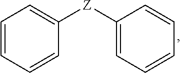

wherein Z is a bond, O, S, NH, CH₂, NHCH₂, or C≡C;
R⁸ is a bond, —O—, or —N(R¹⁷)—, optionally substituted C₁-C₆alkyl, optionally substituted C₁-C₆heteroalkyl, optionally substituted heterocycloalkyl, optionally substituted aryl, or optionally substituted heteroaryl;
R⁹ is —CH₂OH, —CH₂CH(CH₃)₂,

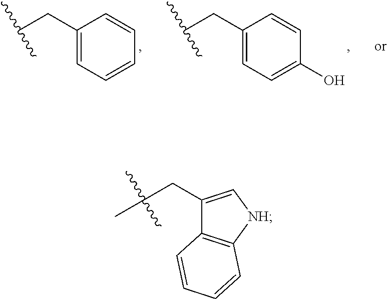 [SMILES: *Cc1ccccc1 ; *Cc1ccc(O)cc1 ; *Cc1c[nH]c2ccccc12]

R¹⁴ is C₁-C₆alkyl, C₁-C₆haloalkyl, —C(O)OR²⁸, —CF₂C(O)OH, or

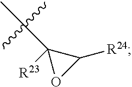

R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

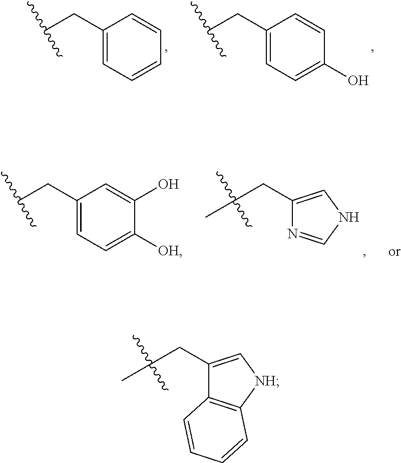 [SMILES: *Cc1ccccc1 ; *Cc1ccc(O)cc1 ; *Cc1ccc(O)c(O)c1 ; *Cc1c[nH]cn1 ; *Cc1c[nH]c2ccccc12]

each R²⁵ is independently C₁-C₆alkyl;
R²⁶ is H, or C₁-C₄alkyl;
R²⁷ is H, or C₁-C₄alkyl;
R²⁸ is C₁-C₆alkyl;
n is 0 or 1;
p is 0 or 1; and
q is 0 or 1;
or a pharmaceutically acceptable salt, solvate, or prodrug thereof.

In one embodiment is a compound of Formula (A) wherein R¹ is

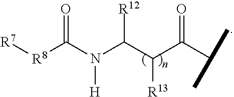

In another embodiment is a compound of Formula (A) wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

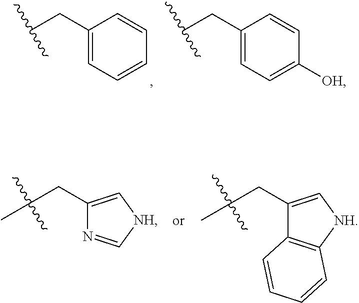

In another embodiment is a compound of Formula (A) wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

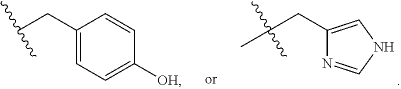 [SMILES: *Cc1ccc(O)cc1 ; "or" ; *Cc1c[nH]cn1]

In another embodiment is a compound of Formula (A) wherein R⁷ is a linear or branched alkyl chain of 1-22 carbon atoms, optionally comprising within the alkyl chain or at an alkyl chain terminus an optionally substituted aryl, an optionally substituted heteroaryl, an optionally substituted heterocycloalkyl, or an optionally substituted

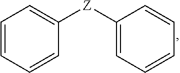

wherein Z is a bond, O, S, NH, CH₂, NHCH₂, or C≡C. In another embodiment is a compound of Formula (A) wherein R⁷ is a linear or branched alkyl chain of 1-22 carbon atoms, optionally comprising within the alkyl chain or at an alkyl chain terminus an optionally substituted

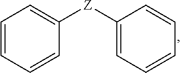

wherein Z is a bond. In another embodiment is a compound of Formula (A) wherein R⁷ is

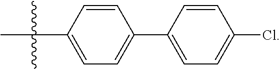 [SMILES: *c1ccc(-c2ccc(Cl)cc2)cc1]

In another embodiment is a compound of Formula (A) wherein R⁸ is a optionally substituted C₁-C₆heteroalkyl. In another embodiment is a compound of Formula (A) wherein R⁸ is a bond. In another embodiment is a compound of Formula (A) wherein R¹⁴ is —C(O)OR²⁸. In another embodiment is a compound of Formula (A) wherein R²⁸ is —CH₃. In another embodiment is a compound of Formula (A) wherein R¹⁴ is

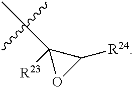

In another embodiment is a compound of Formula (A) wherein R²³ is H or C₁-C₄alkyl; and R²⁴ is H or optionally substituted C₁-C₈alkyl. In another embodiment is a compound of Formula (A) wherein R²³ and R²⁴ are each H. In another embodiment is a compound of Formula (A) wherein R²³ is H and R²⁴ is CH₃. In another embodiment is a compound of Formula (A) wherein R²³ is CH₃ and R²⁴ is H. In another embodiment is a compound of Formula (A) wherein R¹⁴ is C₁-C₆haloalkyl. In another embodiment is a compound of Formula (A) wherein R¹⁴ is CF₃. In another embodiment is a compound of Formula (A) wherein n is 0.

In another embodiment is a compound of Formula (A) wherein R¹ is

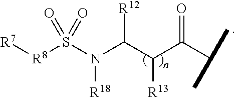

In another embodiment is a compound of Formula (A) wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

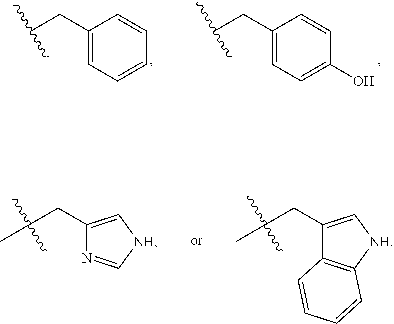 [SMILES: *Cc1ccccc1 ; *Cc1ccc(O)cc1 ; *Cc1c[nH]cn1 ; "or" ; *Cc1c[nH]c2ccccc12]

In another embodiment is a compound of Formula (A) wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

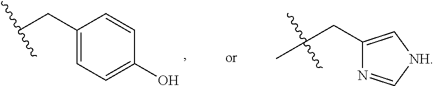 [SMILES: *Cc1ccc(O)cc1 ; "," ; "or" ; *Cc1c[nH]cn1]

In another embodiment is a compound of Formula (A) wherein R⁷ is a linear or branched alkyl chain of 1-22 carbon atoms, optionally comprising within the alkyl chain or at an alkyl chain terminus an optionally substituted aryl, an optionally substituted heteroaryl, an optionally substituted heterocycloalkyl, or an optionally substituted

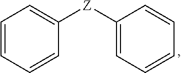

wherein Z is a bond, O, S, NH, CH₂, NHCH₂, or C≡C. In another embodiment is a compound of Formula (A) wherein R⁷ is a linear or branched alkyl chain of 1-22 carbon atoms, optionally comprising within the alkyl chain or at an alkyl chain terminus an optionally substituted

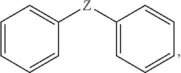

wherein Z is a bond. In another embodiment is a compound of Formula (A) wherein R⁷ is

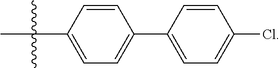 [SMILES: *c1ccc(-c2ccc(Cl)cc2)cc1]

In another embodiment is a compound of Formula (A) wherein R⁸ is a optionally substituted C₁-C₆heteroalkyl. In another embodiment is a compound of Formula (A) wherein R⁸ is a bond. In another embodiment is a compound of Formula (A) wherein R¹⁴ is —C(O)OR²⁸. In another embodiment is a compound of Formula (A) wherein R²⁸ is —CH₃. In another embodiment is a compound of Formula (A) wherein R¹⁴ is

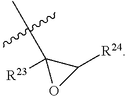

In another embodiment is a compound of Formula (A) wherein R²³ is H or C₁-C₄alkyl; and R²⁴ is H or optionally substituted C₁-C₈alkyl. In another embodiment is a compound of Formula (A) wherein R²³ and R²⁴ are each H. In another embodiment is a compound of Formula (A) wherein R²³ is H and R²⁴ is CH₃. In another embodiment is a compound of Formula (A) wherein R²³ is CH₃ and R²⁴ is H. In another embodiment is a compound of Formula (A) wherein R¹⁴ is C₁-C₆haloalkyl. In another embodiment is a compound of Formula (A) wherein R¹⁴ is CF₃. In another embodiment is a compound of Formula (A) wherein n is 0.

In another aspect is a hydrate or metabolite of a compound of Formula (A).

In another aspect is a pharmaceutical composition comprising a compound of Formula (A) and a pharmaceutically acceptable excipient.

In another aspect is the use of a compound of Formula (A) or a pharmaceutically acceptable salt, pharmaceutically acceptable solvate, or pharmaceutically acceptable prodrug thereof, for the preparation of a medicament for the treatment of a bacterial infection in a patient.

In one aspect is a method for treating a bacterial infection in a mammal comprising administering to the mammal a compound of Formula (A) or a pharmaceutically acceptable salt or prodrug thereof at a frequency and for a duration sufficient to provide a beneficial effect to the mammal. In another embodiment, the bacterial infection is an infection involving Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In another embodiment the bacterial infection is an infection involving a Gram-negative bacteria. In another embodiment, administering comprises a topical administration.

In a further embodiment are methods of treating a mammal in need of such treatment comprising administering to the mammal a second therapeutic agent. In another embodiment, the second therapeutic agent is not an SpsB inhibitor. In another embodiment, the second therapeutic agent is an aminoglycoside antibiotic, fluoroquinolone antibiotic, β-lactam antibiotic, macrolide antibiotic, glycopeptide antibiotic, rifampicin, chloramphenicol, fluoramphenicol, colistin, mupirocin, bacitracin, daptomycin, or linezolid. In another embodiment, the second therapeutic agent is a β-lactam antibiotic. In another embodiment, the β-lactam antibiotic is selected from penicillins, monobactams, cephalosporins, and carbapenems. A further embodiment comprises administering a β-lactamase inhibitor.

In another aspect described herein are compounds having the structure of Formula (XIV):

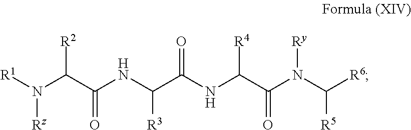

wherein:
R¹ is selected from:

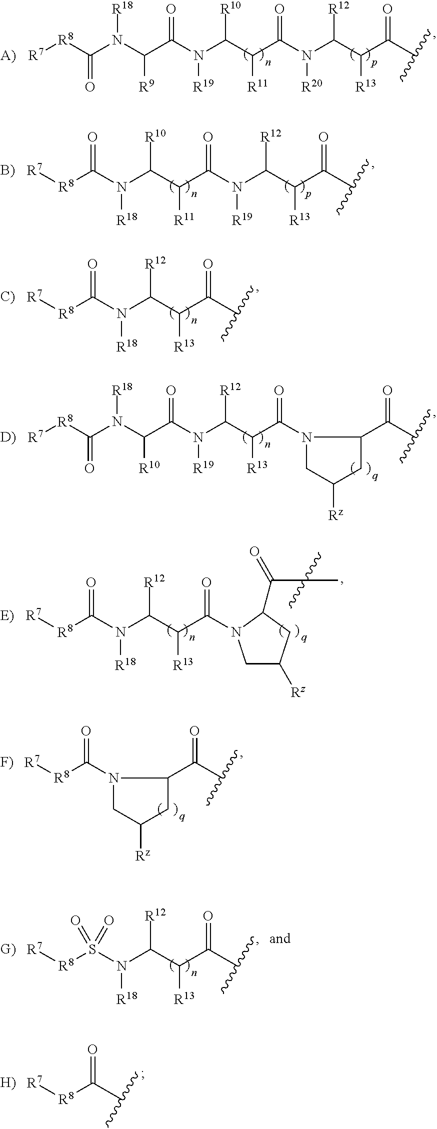

R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

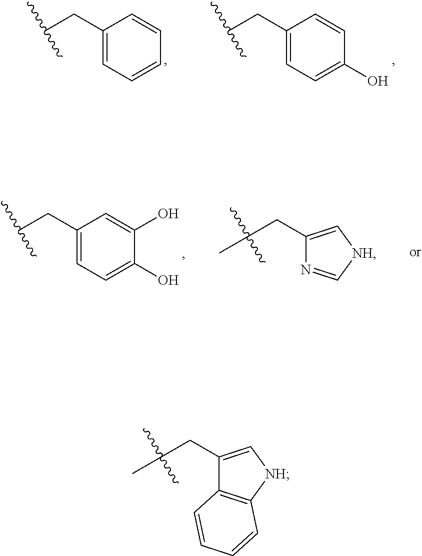 [SMILES: *Cc1ccccc1 ; *Cc1ccc(O)cc1 ; *Cc1ccc(O)c(O)c1 ; *Cc1c[nH]cn1 ; "or" ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —C(═O)H, —CH₂C(═O)H, —C(═O)NHCH₂C(═O)H, —C(═O)C(═O)N(R¹⁴)₂, —C(═O)C(═O)OH, —B(OR²³)(OR²⁴), or

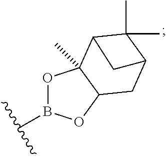 [SMILES: *B1OC2CC3CC(C3(C)C)[C@]2(C)O1]

or R⁵ and R⁶ together with the carbon atom form

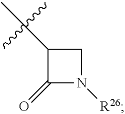

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is unsubstituted C₁-C₁₀alkyl;
R⁸ is optionally substituted C₁-C₁₀heteroalkyl;
R⁹ is —CH₂OH, —CH₂CH(CH₃)₂,

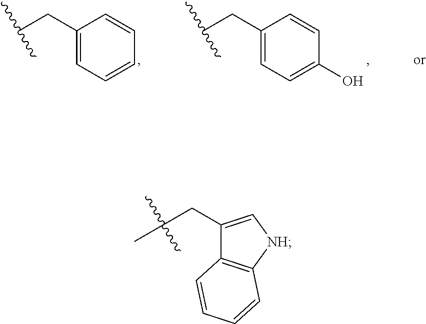 [SMILES: *Cc1ccccc1 ; *Cc1ccc(O)cc1 ; *Cc1c[nH]c2ccccc12]

R¹⁴, R¹⁵, and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ and R²⁴ are each independently H, or C₁-C₄alkyl; or R²³ and R²⁴ together with the boron atom form an optionally substituted 5- or 6-membered boron containing ring;
each R²⁵ is independently C₁-C₆alkyl;
R²⁶ is H, C₁-C₄alkyl, C₁-C₄alkoxy, —CH₂C(O)OR²⁵, or —OCH₂C(O)OR²⁵;
n is 0 or 1;
p is 0 or 1; and
q is 0 or 1;
or a pharmaceutically acceptable salt, solvate, or prodrug thereof.

In one embodiment is a compound of Formula (XIV) wherein R¹ is

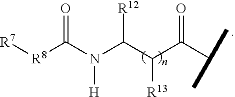

In another embodiment is a compound of Formula XIV) wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

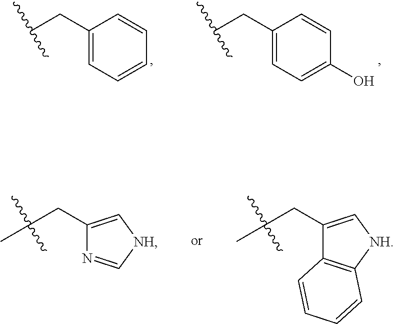 [SMILES: *Cc1ccccc1 ; *Cc1ccc(O)cc1 ; *Cc1c[nH]cn1 ; "or" ; *Cc1c[nH]c2ccccc12]

In another embodiment is a compound of Formula (XIV) wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

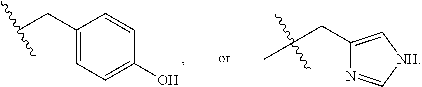 [SMILES: *Cc1ccc(O)cc1 ; "or" ; *Cc1c[nH]cn1]

In another embodiment is a compound of Formula (XIV) wherein R⁷ is unsubstituted C₁-C₈alkyl. In another embodiment is a compound of Formula (XIV) wherein R⁸ is a substituted C₁-C₈heteroalkyl. In another embodiment is a compound of Formula (XIV) wherein R⁸ is an unsubstituted C₁-C₈heteroalkyl. In another embodiment is a compound of Formula (XIV) wherein R⁶ is

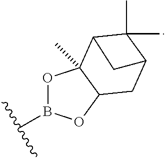 [SMILES: *B1OC2CC3CC(C3(C)C)[C@]2(C)O1]

In another embodiment is a compound of Formula (XIV) wherein R⁶ is —B(OH)₂. In another embodiment is a compound of Formula (XIV) wherein R⁶ is —C(═O)C(═O)OH. In another embodiment is a compound of Formula (XIV) wherein n is 0.

In one embodiment is a compound of Formula (XIV) wherein R¹ is

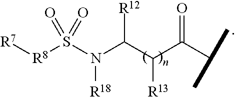

In another embodiment is a compound of Formula XIV) wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

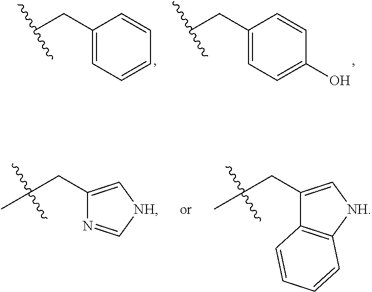

In another embodiment is a compound of Formula (XIV) wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

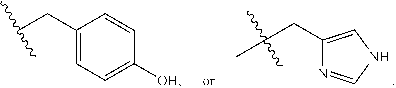 [SMILES: *Cc1ccc(O)cc1 ; "or" ; *Cc1c[nH]cn1 ; "."]

In another embodiment is a compound of Formula (XIV) wherein R⁷ is unsubstituted C₁-C₈alkyl. In another embodiment is a compound of Formula (XIV) wherein R⁸ is a substituted C₁-C₈heteroalkyl. In another embodiment is a compound of Formula (XIV) wherein R⁸ is an unsubstituted C₁-C₈heteroalkyl. In another embodiment is a compound of Formula (XIV) wherein R⁶ is

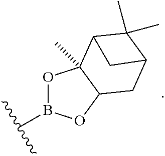 [SMILES: *B1OC2CC3CC(C3(C)C)[C@]2(C)O1]

In another embodiment is a compound of Formula (XIV) wherein R⁶ is —B(OH)₂. In another embodiment is a compound of Formula (XIV) wherein R⁶ is —C(═O)C(═O)OH. In another embodiment is a compound of Formula (XIV) wherein n is 0.

In another aspect is a hydrate or metabolite of a compound of Formula (XIV).

In another aspect is a pharmaceutical composition comprising a compound of Formula (XIV) and a pharmaceutically acceptable excipient.

In another aspect is the use of a compound of Formula (XIV) or a pharmaceutically acceptable salt, pharmaceutically acceptable solvate, or pharmaceutically acceptable prodrug thereof, for the preparation of a medicament for the treatment of a bacterial infection in a patient.

In one aspect is a method for treating a bacterial infection in a mammal comprising administering to the mammal a compound of Formula (XIV) or a pharmaceutically acceptable salt or prodrug thereof at a frequency and for a duration sufficient to provide a beneficial effect to the mammal. In another embodiment, the bacterial infection is an infection involving Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In another embodiment the bacterial infection is an infection involving a Gram-negative bacteria. In another embodiment, administering comprises a topical administration.

In a further embodiment are methods of treating a mammal in need of such treatment comprising administering to the mammal a second therapeutic agent. In another embodiment, the second therapeutic agent is not an SpsB inhibitor. In another embodiment, the second therapeutic agent is an aminoglycoside antibiotic, fluoroquinolone antibiotic, β-lactam antibiotic, macrolide antibiotic, glycopeptide antibiotic, rifampicin, chloramphenicol, fluoramphenicol, colistin, mupirocin, bacitracin, daptomycin, or linezolid. In another embodiment, the second therapeutic agent is a β-lactam antibiotic. In another embodiment, the β-lactam antibiotic is selected from penicillins, monobactams, cephalosporins, and carbapenems. A further embodiment comprises administering a β-lactamase inhibitor.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 depicts synergy between Compound 217 and imipenem.

FIG. 2 depicts the time-kill assay for Compound 217 and imipenem.

### DETAILED DESCRIPTION OF THE INVENTION

### Definitions

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “disease” or “disorder” or “malcondition” are used interchangeably, and are used to refer to diseases or conditions wherein a bacterial SPase plays a role in the biochemical mechanisms involved in the disease or malcondition such that a therapeutically beneficial effect can be achieved by acting on the enzyme. “Acting on” SPase can include binding to SPase and/or inhibiting the bioactivity of an SPase.

The expression “effective amount”, when used to describe therapy to an individual suffering from a disorder, refers to the amount of a compound described herein that is effective to inhibit or otherwise act on SPase in the individual's tissues wherein SPase involved in the disorder is active, wherein such inhibition or other action occurs to an extent sufficient to produce a beneficial therapeutic effect.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds described herein are outweighed by the therapeutically beneficial effects.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

All chiral, diastereomeric, racemic forms of a structure are intended, unless a particular stereochemistry or isomeric form is specifically indicated. Compounds described herein can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

The inclusion of an isotopic form of one or more atoms in a molecule that is different from the naturally occurring isotopic distribution of the atom in nature is referred to as an “isotopically labeled form” of the molecule. All isotopic forms of atoms are included as options in the composition of any molecule, unless a specific isotopic form of an atom is indicated. For example, any hydrogen atom or set thereof in a molecule can be any of the isotopic forms of hydrogen, i.e., protium (¹H), deuterium (²H), or tritium (³H) in any combination. Similarly, any carbon atom or set thereof in a molecule can be any of the isotopic form of carbons, such as ¹¹C, ¹²C, ¹³C, or ¹⁴C, or any nitrogen atom or set thereof in a molecule can be any of the isotopic forms of nitrogen, such as ¹⁴N, ¹³N, ¹⁴N, or ¹⁵N. A molecule can include any combination of isotopic forms in the component atoms making up the molecule, the isotopic form of every atom forming the molecule being independently selected. In a multi-molecular sample of a compound, not every individual molecule necessarily has the same isotopic composition. For example, a sample of a compound can include molecules containing various different isotopic compositions, such as in a tritium or ¹⁴C radiolabeled sample where only some fraction of the set of molecules making up the macroscopic sample contains a radioactive atom. It is also understood that many elements that are not artificially isotopically enriched themselves are mixtures of naturally occurring isotopic forms, such as ¹⁴N and ¹⁵N, ³²S and ³⁴S, and so forth. A molecule as recited herein is defined as including isotopic forms of all its constituent elements at each position in the molecule. As is well known in the art, isotopically labeled compounds can be prepared by the usual methods of chemical synthesis, except substituting an isotopically labeled precursor molecule. The isotopes, radiolabeled or stable, can be obtained by any method known in the art, such as generation by neutron absorption of a precursor nuclide in a nuclear reactor, by cyclotron reactions, or by isotopic separation such as by mass spectrometry. The isotopic forms are incorporated into precursors as required for use in any particular synthetic route. For example, ¹⁴C and ³H can be prepared using neutrons generated in a nuclear reactor. Following nuclear transformation, ¹⁴C and ³H are incorporated into precursor molecules, followed by further elaboration as needed.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R)C(O)R′, (CH₂)₀₋₂N(R)N(R)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” group.

Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

C(O) and S(O)₂ groups can be bound to one or two heteroatoms, such as nitrogen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a urea. When a S(O)₂ group is bound to one carbon and one nitrogen atom, the resulting unit is termed a “sulfonamide.” When a S(O)₂ group is bound to two nitrogen atoms, the resulting unit is termed a “sulfamate.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

Selected substituents within the compounds described herein are present to a recursive degree. In this context, “recursive substituent” means that a substituent may recite another instance of itself or of another substituent that itself recites the first substituent. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

The term “alkyl” refers to a straight or branched hydrocarbon chain radical group consisting solely of carbon and hydrogen atoms from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed above, for example, amino, hydroxy, cyano, carboxy, nitro, thio, alkoxy, and halogen groups. A description herein that a group is an alkyl chain “optionally comprising within the alkyl chain or at an alkyl chain terminus”, signifies that a moiety can be disposed between two subunits of the alkyl chain, or can be disposed at an unsubstituted end of the chain, or can be disposed between the chain and a point of attachment of the chain, for example to a carbonyl, NR, or O group. For example, an alkylbenzoyl group is an alkyl chain with a phenyl group disposed between the alkyl and a carbonyl, fitting the above description; an N-alkylphenylcarboxamido is an alkyl chain with a phenyl group disposed between the alkyl and the aminocarbonyl group, filling within the above description.

The term “alkylene” means a linear saturated divalent hydrocarbon radical of one to six carbon atoms or a branched saturated divalent hydrocarbon radical of one to six carbon atoms unless otherwise stated, such as methylene, ethylene, propylene, 1-methylpropylene, 2-methylpropylene, butylene, pentylene, and the like.

The term “carbonyl” means C═O.

The terms “carboxy” and “hydroxycarbonyl” mean COOH.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic,” “carbocyclyl,” and “carbocycle” denote a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂CH₂—S(═O)—CH₃, and —CH₂CH₂—O—CH₂CH₂—O—CH₃. Up to two heteroatoms may be consecutive, such as, for example, —CH₂—NH—OCH₃, or —CH₂—CH₂—S—S—CH₃.

A “cycloheteroalkyl” ring or “heterocycloalkyl” ring is a cycloalkyl ring containing at least one heteroatom. A cycloheteroalkyl ring can also be termed a “heterocyclyl,” described below.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and —CH═CH—O—CH₂CH₂—O—CH₃.

Aryl groups are cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups or the term “heterocyclyl” includes aromatic and non-aromatic ring compounds containing 3 or more ring members, of which, one or more is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The term “thioalkoxy” refers to an alkyl group previously defined attached to the parent molecular moiety through a sulfur atom.

The term “glycosyloxyoxy” refers to a glycoside attached to the parent molecular moiety through an oxygen atom.

The term “alkoxycarbonyl” represents as ester group; i.e. an alkoxy group, attached to the parent molecular moiety through a carbonyl group such as methoxycarbonyl, ethoxycarbonyl, and the like.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary or quaternary amino group. An “alkylamino” group includes a monoalkylamino, dialkylamino, and trialkylamino group.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to primary carboxamide groups (—C(O)NH₂) and formamide groups (—NHC(O)H). A “carboxamido” or “aminocarbonyl” group is a group of the formula C(O)NR₂, wherein R can be H, alkyl, aryl, etc.

The term “azido” refers to an N₃ group. An “azide” can be an organic azide or can be a salt of the azide (N₃) anion. The term “nitro” refers to an NO₂ group bonded to an organic moiety. The term “nitroso” refers to an NO group bonded to an organic moiety. The term nitrate refers to an ONO₂ group bonded to an organic moiety or to a salt of the nitrate (NO₃) anion.

The term “urethane” (“carbamoyl” or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds described herein may take the form of salts. The term “salts” embraces addition salts of free acids or free bases which are compounds described herein. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compounds of the present disclosure.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the present disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) compounds, for example in their purification by recrystallization. All of these salts may be prepared by conventional means from the corresponding compound according to Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) by reacting, for example, the appropriate acid or base with the compound according to Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The composition can include water in stoichiometic quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometic or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within the patients body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

In addition, where features or aspects of the present disclosure are described in terms of Markush groups, those skilled in the art will recognize that the presently described compounds is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the present disclosure are described in terms of Markush groups, those skilled in the art will recognize that the present disclosure is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The present disclosure further embraces isolated compounds according to Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′). The expression “isolated compound” refers to a preparation of a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′), or a mixture of compounds according to Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′), wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a mixture of compounds according to Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′), which contains the named compound or mixture of compounds according to Formula (A) in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds described herein and intermediates may be isolated from their reaction mixtures and purified by standard techniques such as filtration, liquid-liquid extraction, solid phase extraction, distillation, recrystallization or chromatography, including flash column chromatography, or HPLC.

### Isomerism and Tautomerism in Compounds Described Herein

### Tautomerism

Within the present disclosure it is to be understood that a compound of the Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the present disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

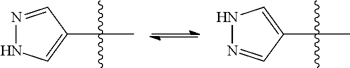 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

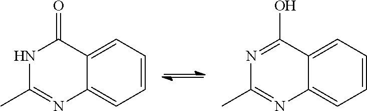

is an example of tautomerism. Accordingly, a structure depicted herein as one tautomer is intended to also include the other tautomer.

### Optical Isomerism

It will be understood that when compounds of the present disclosure contain one or more chiral centers, the compounds may exist in, and may be isolated as pure enantiomeric or diastereomeric forms or as racemic mixtures. The present disclosure therefore includes any possible enantiomers, diastereomers, racemates or mixtures thereof of the compounds described herein.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

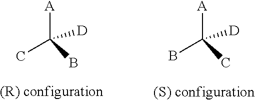

The present disclosure is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound described herein, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (see below). It is further understood that certain structural elements, including steric bulk or substituents on the amide nitrogen, may enhance the stability of a rotamer to the extent that a compound may be isolated as, and exist indefinitely, as a single stable rotamer. The present disclosure therefore includes any possible stable rotamers of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) which are biologically active in the treatment of cancer or other proliferative disease states.

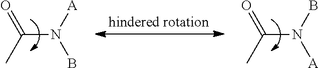

### Regioisomerism

In some embodiments, the compounds described herein have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below.

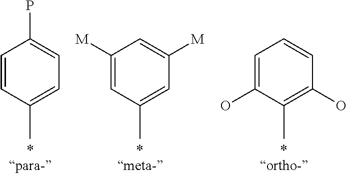

In various embodiments, the compound or set of compounds, such as are among the inventive compounds or are used in the inventive methods, can be any one of any of the combinations and/or sub-combinations of the above-listed embodiments.

### Compounds

In one aspect described herein are compounds of Formula (A):

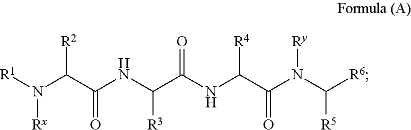

wherein:
R¹ is selected from:

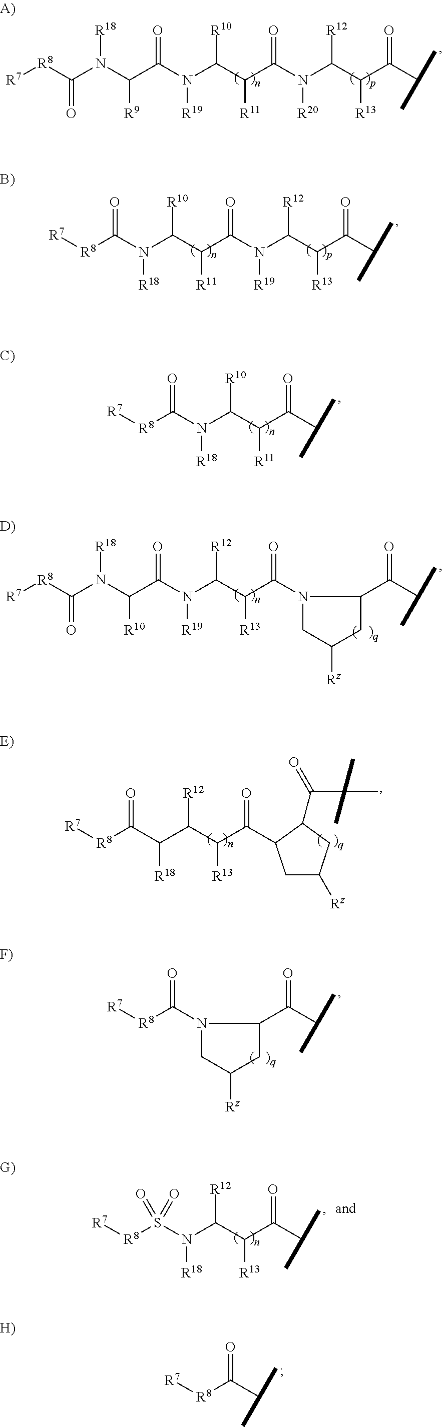

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

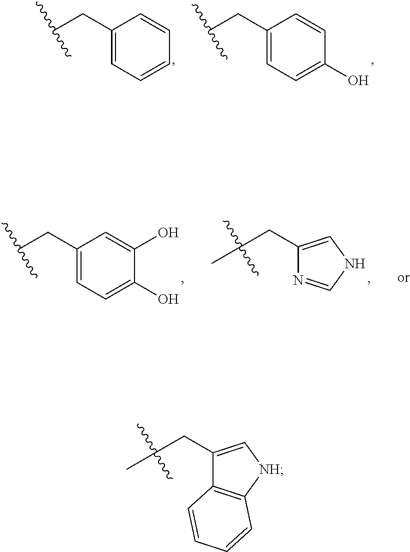

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heterocycloalkyl, or an optionally substituted

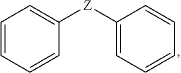

wherein Z is a bond, O, S, NH, CH₂, NHCH₂, or C≡C;
R⁸ is a bond, —O—, or —N(R¹⁷)—, optionally substituted C₁-C₆alkyl, optionally substituted C₁-C₆heteroalkyl, optionally substituted heterocycloalkyl, optionally substituted aryl, or optionally substituted heteroaryl;
R⁹ is —CH₂OH, —CH₂CH(CH₃)₂,

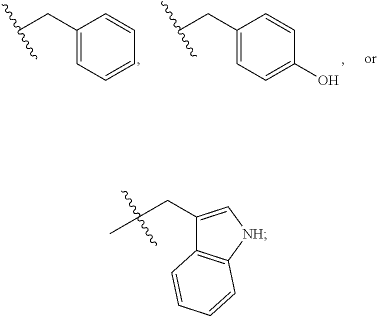

R¹⁴ is C₁-C₆alkyl, C₁-C₆haloalkyl, —C(O)OR²⁸, —CF₂C(O)OH, or

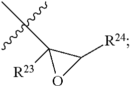

R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

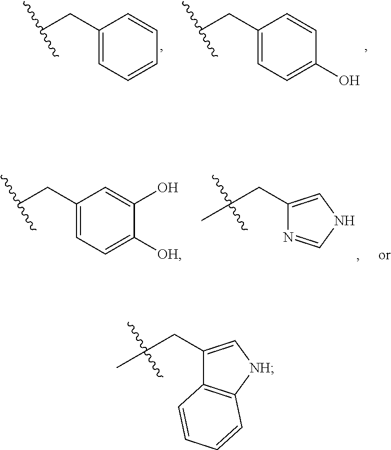

each R²⁵ is independently C₁-C₆alkyl;
R²⁶ is H, or C₁-C₄alkyl;
R²⁷ is H, or C₁-C₄alkyl;
R²⁸ is C₁-C₆alkyl;
n is 0 or 1;
p is 0 or 1; and
q is 0 or 1;
or a pharmaceutically acceptable salt, solvate, or prodrug thereof

In another embodiment is a compound of Formula (A) having the structure of Formula (A′):

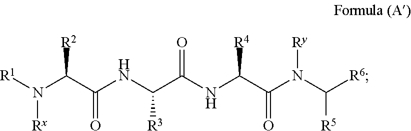

wherein:
R¹ is selected from:

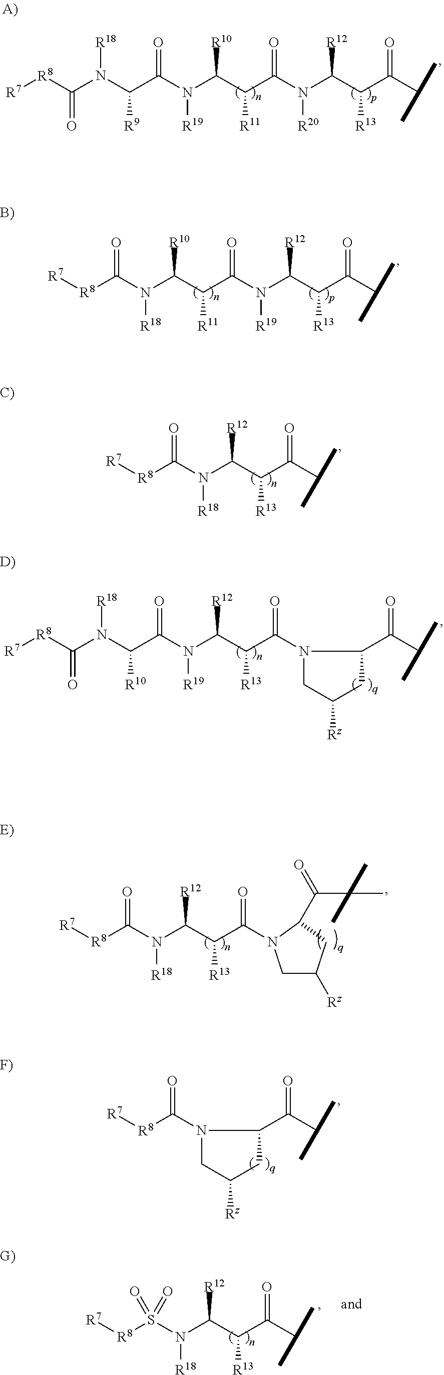

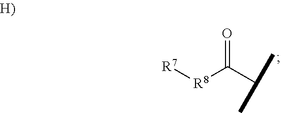

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

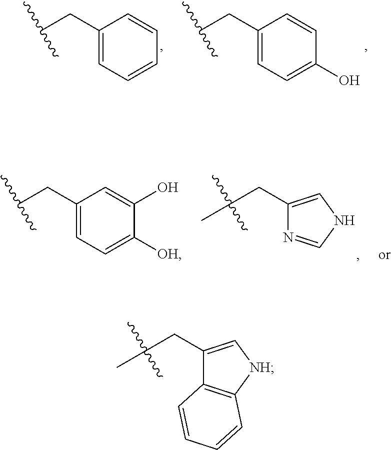

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heterocycloalkyl, or an optionally substituted

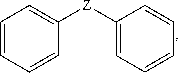

wherein Z is a bond, O, S, NH, CH₂, NHCH₂, or C≡C;
R⁸ is a bond, —O—, or —N(R¹⁷)—, optionally substituted C₁-C₆alkyl, optionally substituted C₁-C₆heteroalkyl, optionally substituted heterocycloalkyl, optionally substituted aryl, or optionally substituted heteroaryl;
R⁹ is —CH₂OH, —CH₂CH(CH₃)₂,

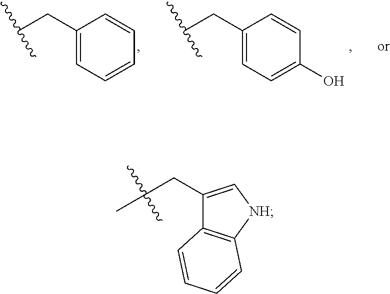 [SMILES: *Cc1ccccc1 ; *Cc1ccc(O)cc1 ; *Cc1c[nH]c2ccccc12]

R¹⁴ is C₁-C₆alkyl, C₁-C₆haloalkyl, —C(O)OR²⁸, —CF₂C(O)OH, or

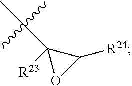

R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

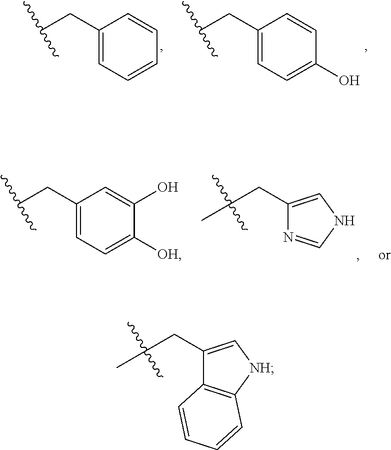

each R²⁵ is independently C₁-C₆alkyl;
R²⁶ is H, or C₁-C₄alkyl;
R²⁷ is H, or C₁-C₄alkyl;
R²⁸ is C₁-C₆alkyl;
n is 0 or 1;
p is 0 or 1; and
q is 0 or 1;
or a pharmaceutically acceptable salt, solvate, or prodrug thereof

In another embodiment is a compound of Formula (A) or Formula (A′) wherein R¹ is

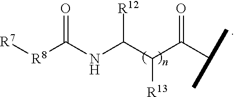

In another embodiment is a compound of Formula (A) or Formula (A′) wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

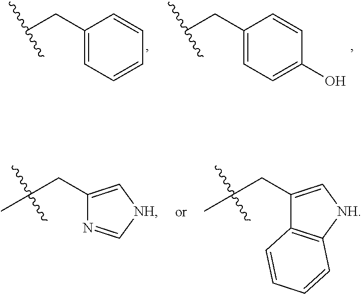

In a further embodiment is a compound of Formula (A) or Formula (A′) wherein R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

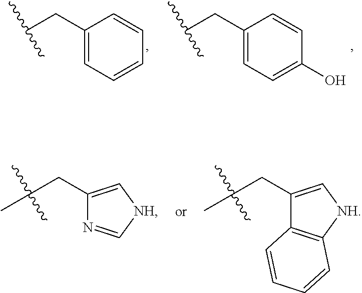

In yet a further embodiment is a compound of Formula (A) or Formula (A′) wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

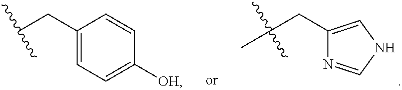 [SMILES: *Cc1ccc(O)cc1 ; "or" ; *Cc1c[nH]cn1]

In a further embodiment is a compound of Formula (A) or Formula (A′) wherein R⁸ is a bond. In a further embodiment is a compound of Formula (A) or Formula (A′) wherein R⁸ is an optionally substituted C₁-C₆heteroalkyl. In another embodiment is a compound of Formula (A) or Formula (A′) wherein R¹⁴ is —C(O)OR²⁸. In another embodiment is a compound of Formula (A) or Formula (A′) wherein R¹⁴ is —C(O)OR²⁸ and R²⁸ is —CH₃. In another embodiment is a compound of Formula (A) or Formula (A′) wherein R¹⁴ is —C(O)OR²⁸ and R²⁸ is —CH₂CH₃. In another embodiment is a compound of Formula (A) or Formula (A′) wherein R¹⁴ is

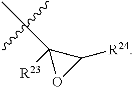

In another embodiment is a compound of Formula (A) or Formula (A′) wherein R²³ is H or C₁-C₄alkyl; and R²⁴ is H or optionally substituted C₁-C₈alkyl. In another embodiment is a compound of Formula (A) or Formula (A′) wherein R²³ and R²⁴ are each H. In another embodiment is a compound of Formula (A) or Formula (A′) wherein R²³ is H and R²⁴ is CH₃. In another embodiment is a compound of Formula (A) or Formula (A′) wherein R²³ is CH₃ and R²⁴ is H. In another embodiment is a compound of Formula (A) or Formula (A′) wherein R¹⁴ is C₁-C₆alkyl. In another embodiment is a compound of Formula (A) or Formula (A′) wherein R¹⁴ is CH₃. In another embodiment is a compound of Formula (A) or Formula (A′) wherein R¹⁴ is C₁-C₆haloalkyl. In another embodiment is a compound of Formula (A) or Formula (A′) wherein R¹⁴ is CF₃. In a further embodiment of the aforementioned embodiments is a compound of Formula (A) or Formula (A′) wherein n is 0. In yet a further embodiment, n is 1.

In a further embodiment is a compound of Formula (A) or Formula (A′) having the structure of Formula (AA):

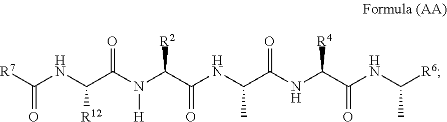

wherein R², R⁴, and R¹² are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂; and R⁶ and R⁷ are defined as above.

In another embodiment is a compound of Formula (AA) wherein R⁴ is —(CH₂)₄NH₂, R² is —CH(OH)(CH₃), and R¹² is —(CH₂)₂NH₂. In another embodiment is a compound of Formula (AA) wherein R⁴ is —(CH₂)₄NH₂, R² is —CH(OH)(CH₃), and R¹² is —CH₂NH₂. In another embodiment is a compound of Formula (AA) wherein R⁴ is —CH₂C(O)NH₂, R² is —CH(OH)(CH₃), and R¹² is —(CH₂)₄NH₂. In another embodiment is a compound of Formula (AA) wherein R⁴ is —(CH₂)₄NH₂, R² is —(CH₂)₄NH₂, and R¹² is —CH₂NH₂. In another embodiment is a compound of Formula (AA) wherein R⁴ is —CH₂C(O)NH₂, R² is —(CH₂)₄NH₂, and R¹² is —CH₂NH₂. In another embodiment is a compound of Formula (AA) wherein R⁴ is —CH₂CH(CH₃)₂, R² is —(CH₂)₂NH₂, and R¹² is —(CH₂)₂NH₂.

In another embodiment is a compound of Formula (A) or Formula (A′) wherein R¹ is

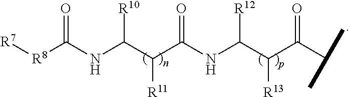

In another embodiment, R⁸ is a bond. In another embodiment, R⁸ is an optionally substituted C₁-C₆heteroalkyl. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

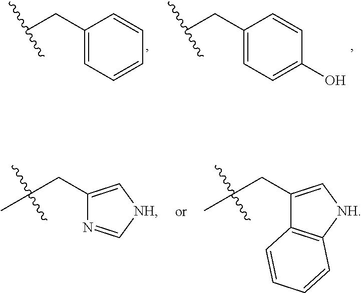

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

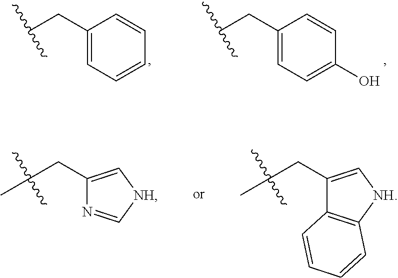 [SMILES: *Cc1ccccc1 ; *Cc1ccc(O)cc1 ; *Cc1c[nH]cn1 ; "or" ; *Cc1c[nH]c2ccccc12]

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂, or

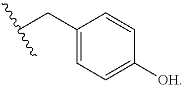 [SMILES: *Cc1ccc(O)cc1]

In a further embodiment of the aforementioned embodiments is a compound of Formula (A) or Formula (A′) wherein n is 0 and p is 0. In another embodiment, n is 0 and p is 1. In yet a further embodiment, n is 1 and p is 0.

In a further embodiment is a compound of Formula (A′) having the structure of Formula (AB):

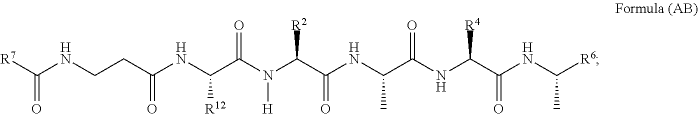

wherein R², R⁴, and R¹², are each independently —CH₂CH(CH₃)₂, —(CH₂)₃NH₂, or —(CH₂)₄NH₂.

In another embodiment is a compound of Formula (AB) wherein R², R⁴, and R¹² are each —(CH₂)₄NH₂. In another embodiment is a compound of Formula (AB) wherein R², R⁴, and R¹² are each —(CH₂)₃NH₂. In another embodiment is a compound of Formula (AB) wherein R⁴ is —CH₂CH(CH₃)₂, R² is —(CH₂)₃NH₂, and R¹² is —(CH₂)₄NH₂. In another embodiment is a compound of Formula (AB) wherein R⁴ is —CH₂CH(CH₃)₂, R² is —(CH₂)₄NH₂, and R¹² is —(CH₂)₄NH₂.

In a further embodiment is a compound of Formula (A′) having the structure of Formula (ABB):

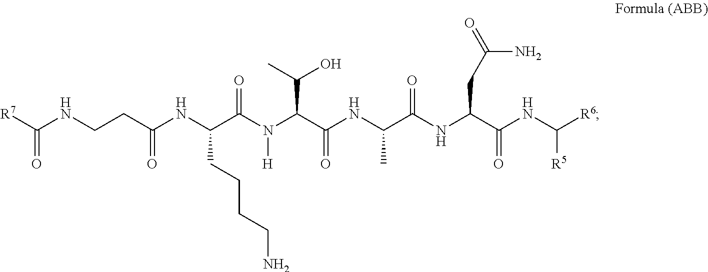

wherein R⁵ is —H, or —CH₃.

In a further embodiment is a compound of Formula (A′) having the structure of Formula (ABBB):

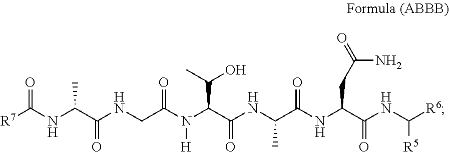

wherein R⁵ is —H, or —CH₃.

In another embodiment is a compound of Formula (A) or Formula (A′) wherein R¹ is

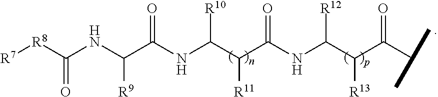

In another embodiment, R⁸ is a bond. In another embodiment, R⁸ is an optionally substituted C₁-C₆heteroalkyl. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

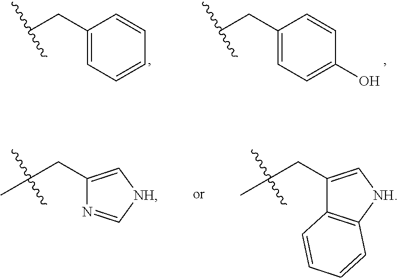 [SMILES: *Cc1ccccc1 ; *Cc1ccc(O)cc1 ; *Cc1c[nH]cn1 ; "or" ; *Cc1c[nH]c2ccccc12]

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

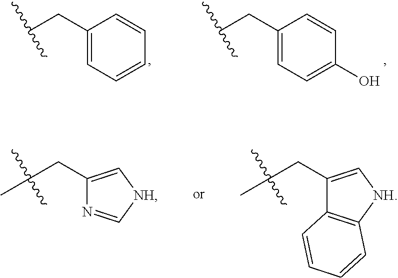 [SMILES: *Cc1ccccc1 ; *Cc1ccc(O)cc1 ; *Cc1c[nH]cn1 ; "or" ; *Cc1c[nH]c2ccccc12]

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

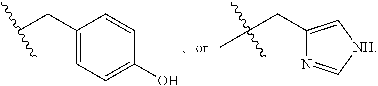 [SMILES: *Cc1ccc(O)cc1 ; *Cc1c[nH]cn1]

In a further embodiment of the aforementioned embodiments is a compound of Formula (A) or Formula (A′) wherein n is 0 and p is 0. In another embodiment, n is 0 and p is 1. In yet a further embodiment, n is 1 and p is 0.

In a further embodiment is a compound of Formula (A′) having the structure of Formula (AC):

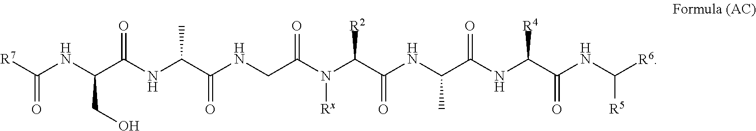

In another embodiment is a compound of Formula (AC) wherein R² is —CH(OH)(CH₃), —CH₂CH₂C(O)OH, or —(CH₂)₄NH₂. In some embodiments, R² is —CH(OH)(CH₃). In some embodiments, R² is —CH₂CH₂C(O)OH. In some embodiments, R² is —(CH₂)₄NH₂. In a further embodiment is a compound of Formula (AC) wherein R⁴ is CH₂CH(CH₃)₂ or —CH₂C(O)NH₂. In some embodiments, R⁴ is CH₂CH(CH₃)₂. In some embodiments, R⁴ is —CH₂C(O)NH₂. In yet a further embodiment is a compound of Formula (AC) wherein R⁵ is H or —CH₃. In some embodiments, R⁴ is H. In some embodiments, R⁴ is —CH₃.

In another embodiment is a compound of Formula (A) or Formula (A′) wherein R¹ is

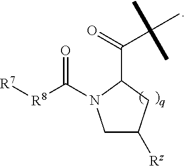

In a further embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

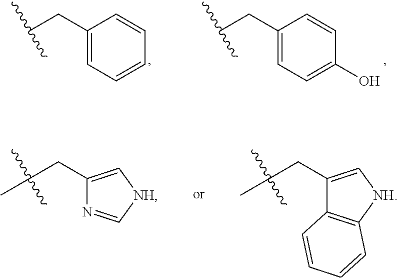 [SMILES: *Cc1ccccc1 ; *Cc1ccc(O)cc1 ; *Cc1c[nH]cn1 ; "or" ; *Cc1c[nH]c2ccccc12]

In another embodiment, q is 1 and R⁸ is a bond. In another embodiment, R⁸ is an optionally substituted C₁-C₆heteroalkyl.

In a further embodiment is a compound of Formula (A′) having the structure of Formula (AD):

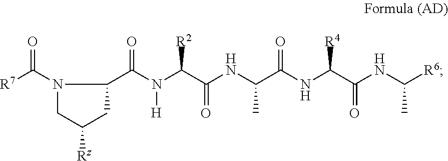

wherein R^{z} is NH₂; and R² and R⁴ are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (AD) wherein R² is —CH(OH)(CH₃), and R⁴ is —CH₂C(O)NH₂. In another embodiment is a compound of Formula (AD) wherein R² is —CH(OH)(CH₃), and R⁴ is —(CH₂)₂NH₂. In another embodiment is a compound of Formula (AD) wherein R² is —CH(OH)(CH₃), and R⁴ is —(CH₂)₃NH₂. In another embodiment is a compound of Formula (AD) wherein R² is —CH(OH)(CH₃), and R⁴ is —(CH₂)₄NH₂. In another embodiment is a compound of Formula (AD) wherein R² is —(CH₂)₄NH₂ and R⁴ is —CH₂CH(CH₃)₂. In another embodiment is a compound of Formula (AD) wherein R² is —(CH₂)₄NH₂ and R⁴ is —CH₂C(O)NH₂. In another embodiment is a compound of Formula (AD) wherein R² is —(CH₂)₄NH₂ and R⁴ is —(CH₂)₄NH₂.

In another embodiment is a compound of Formula (A) or Formula (A′) wherein R¹ is

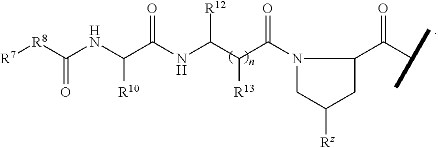

In another embodiment, R⁸ is a bond. In another embodiment, R⁸ is an optionally substituted C₁-C₆heteroalkyl. In another embodiment, R², R⁴, R¹⁰, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

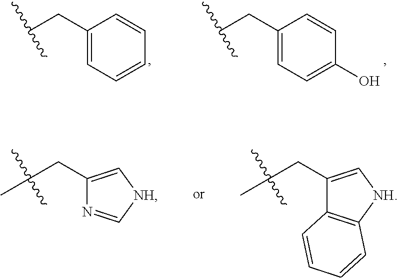 [SMILES: *Cc1ccccc1 ; *Cc1ccc(O)cc1 ; *Cc1c[nH]cn1 ; "or" ; *Cc1c[nH]c2ccccc12]

In a further embodiment, R², R⁴, R¹⁰, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

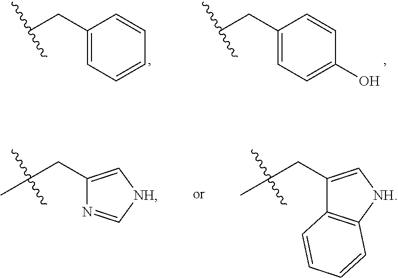 [SMILES: *Cc1ccccc1 ; *Cc1ccc(O)cc1 ; *Cc1c[nH]cn1 ; "or" ; *Cc1c[nH]c2ccccc12]

In yet a further embodiment, R², R⁴, R¹⁰, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

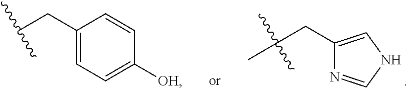 [SMILES: *Cc1ccc(O)cc1 ; "or" ; *Cc1c[nH]cn1]

In a further embodiment of the aforementioned embodiments is a compound of Formula (A) or Formula (A′) wherein n is 0. In yet a further embodiment, n is 1.

In a further embodiment is a compound of Formula (A′) having the structure of Formula (ADD):

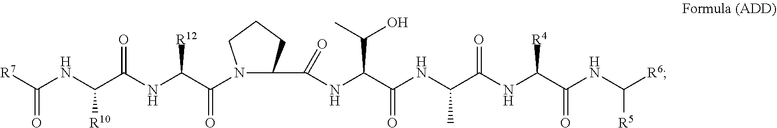

wherein R⁵ is —H, or —CH₃.

In another embodiment is a compound of Formula (ADD) wherein R¹⁰ is —CH₂OH, and R¹² is —CH₃. In another embodiment is a compound of Formula (ADD) wherein R¹⁰ is —CH₂CH(CH₃)₂, and R¹² is —CH(OH)(CH₃). In another embodiment of the aforementioned compounds of Formula (ADD) is a compound wherein R⁴ is —CH₂C(O)NH₂. In yet another embodiment of the aforementioned compounds of Formula (ADD) is a compound wherein R⁴ is

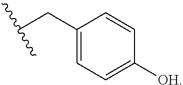 [SMILES: *Cc1ccc(O)cc1]

In another embodiment is a compound of Formula (A) or Formula (A′) wherein R¹ is

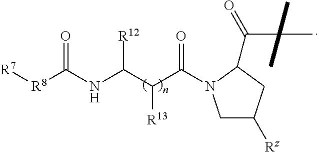

In another embodiment, R⁸ is a bond. In another embodiment, R⁸ is an optionally substituted C₁-C₆heteroalkyl. In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

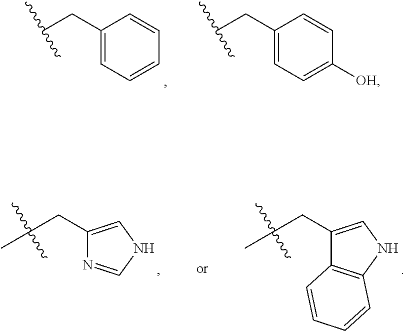 [SMILES: *Cc1ccccc1 ; *Cc1ccc(O)cc1 ; *Cc1c[nH]cn1 ; "or" ; *Cc1c[nH]c2ccccc12]

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

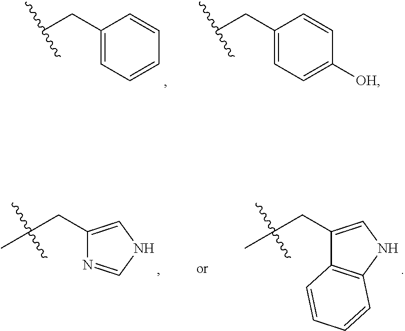 [SMILES: *Cc1ccccc1 ; *Cc1ccc(O)cc1 ; *Cc1c[nH]cn1 ; "or" ; *Cc1c[nH]c2ccccc12]

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂NH₂, —CH₂CH₂C(O)NH₂, —(CH₂)₂NH₂, —(CH₂)₃NH₂, —(CH₂)₄NH₂,

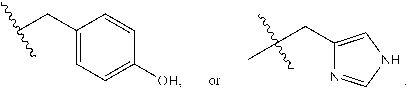 [SMILES: *Cc1ccc(O)cc1 ; "or" ; *Cc1c[nH]cn1]

In a further embodiment of the aforementioned embodiments is a compound of Formula (A) or Formula (A′) wherein n is 0. In yet a further embodiment, n is 1.

In another embodiment is a compound of Formula (A) or Formula (A′) wherein R¹ is

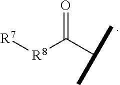

In another embodiment, R⁸ is a bond. In another embodiment, R⁸ is an optionally substituted C₁-C₆heteroalkyl. In another embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

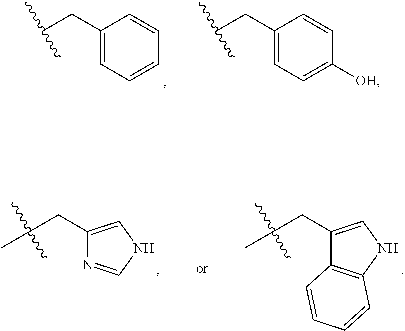 [SMILES: *Cc1ccccc1 ; *Cc1ccc(O)cc1 ; *Cc1c[nH]cn1 ; "or" ; *Cc1c[nH]c2ccccc12]

In a further embodiment, R² and R⁴ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

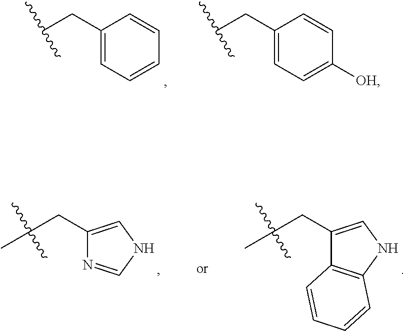 [SMILES: *Cc1ccccc1 ; *Cc1ccc(O)cc1 ; *Cc1c[nH]cn1 ; "or" ; *Cc1c[nH]c2ccccc12]

In yet a further embodiment, R² and R⁴ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

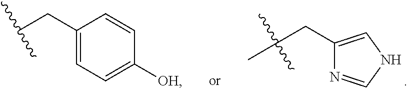 [SMILES: *Cc1ccc(O)cc1 ; "or" ; *Cc1c[nH]cn1]

In another embodiment is a compound of Formula (A) or Formula (A′) wherein R^{x} and R² together with the nitrogen atom form an optionally substituted nitrogen containing ring. In a further embodiment is a compound of Formula (A′) having the structure of Formula (AE):

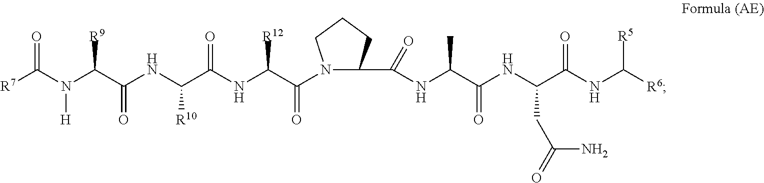

wherein R⁵ is —H, or —CH₃.

In another embodiment is a compound of Formula (AE) wherein R¹⁰ and R¹² are each independently —H, —CH₃, —CH₂CH(CH₃)₂, —CH₂OH, or —CH(OH)(CH₃).

In another aspect described herein are compounds of Formula (I):

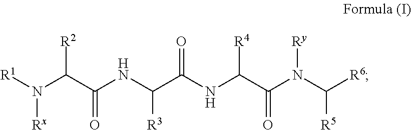

wherein:
R¹ is selected from:

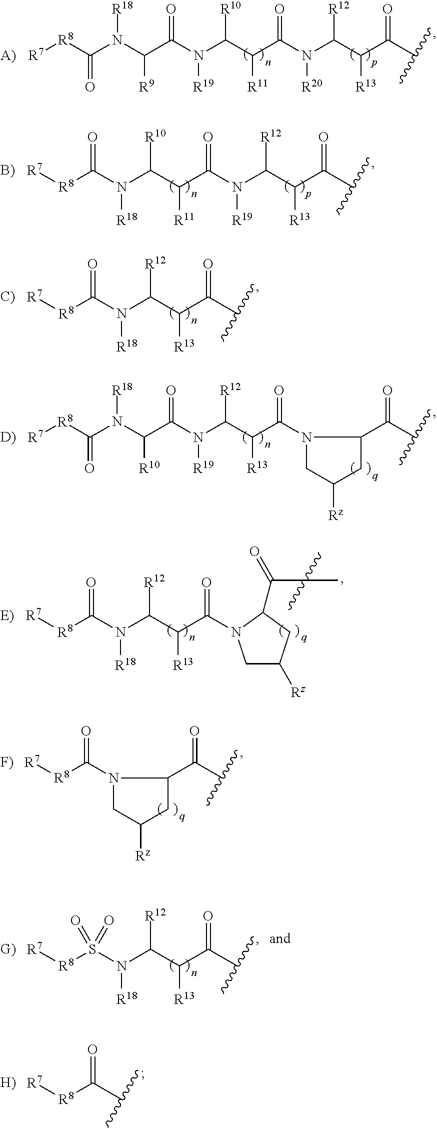

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

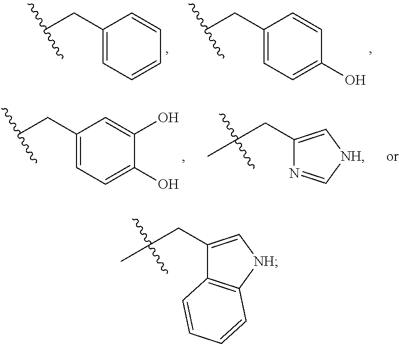

R³ is methyl, ethyl, isopropyl, or cyclopropyl;
R⁵ is H, methyl, ethyl, or —CH₂OH;
R⁶ is —C(═O)C(═O)N(R²³)(R²⁴) or

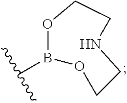 [SMILES: *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

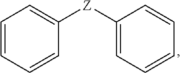

wherein Z is a bond, O, S, NH, CH₂, NHCH₂, or C≡C;
R⁸ is a bond, —O—, or —N(R¹⁷)—, optionally substituted heterocycloalkyl, optionally substituted aryl, or optionally substituted heteroaryl;
R⁹ is —CH₂OH, —CH₂CH(CH₃)₂,

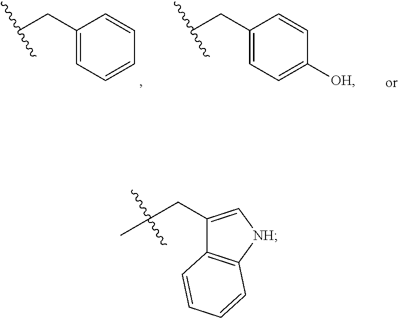 [SMILES: *Cc1ccccc1 ; *Cc1ccc(O)cc1 ; "or" ; *Cc1c[nH]c2ccccc12]

R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ is H, or C₁-C₄alkyl;
R²⁴ is optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²⁵ is independently C₁-C₆alkyl;
R²⁶ is H, or optionally substituted C₁-C₆alkyl;
R²⁷ is optionally substituted heterocycloalkyl, optionally substituted aryl, or optionally substituted heteroaryl;
n is 0 or 1;
p is 0 or 1; and
q is 0 or 1;
or a pharmaceutically acceptable salt, solvate, or prodrug thereof.

In one embodiment is a compound of Formula (I) wherein R¹ is

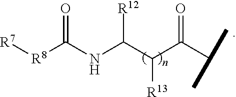

In a further embodiment is a compound of Formula (I) wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

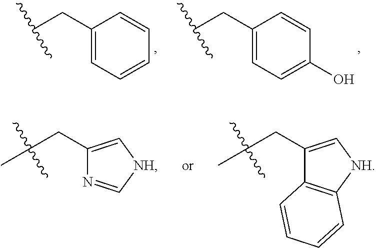

In a further embodiment is a compound of Formula (I) wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

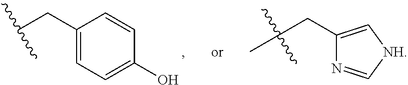 [SMILES: *Cc1ccc(O)cc1 ; *Cc1c[nH]cn1]

In a further embodiment is a compound of Formula (I) wherein R⁸ is a bond. In a further embodiment is a compound of Formula (I) wherein R⁷ is a linear or branched alkyl chain of 1-22 carbon atoms, optionally comprising within the alkyl chain or at an alkyl chain terminus an optionally substituted aryl, an optionally substituted heteroaryl, an optionally substituted heterocycloalkyl, or an optionally substituted

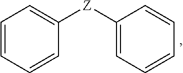

wherein Z is a bond, O, S, NH, CH₂, NHCH₂, or C≡C. In a further embodiment is a compound of Formula (I) wherein R⁷ is a linear or branched alkyl chain of 1-22 carbon atoms, optionally comprising within the alkyl chain or at an alkyl chain terminus an optionally substituted

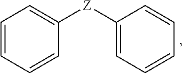

wherein Z is a bond. In a further embodiment is a compound of Formula (I) wherein R⁷ is

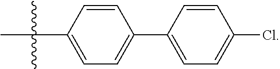 [SMILES: *c1ccc(-c2ccc(Cl)cc2)cc1]

In a further embodiment of the aforementioned embodiments of Formula (I) is a compound wherein R⁶ is —C(═O)C(═O)N(R²³)(R²⁴). In a further embodiment of the aforementioned embodiments of Formula (I) is a compound wherein R⁶ is —C(═O)C(═O)N(R²³)(R²⁴) and R²³ is H. In a further embodiment of the aforementioned embodiments of Formula (I) is a compound wherein R⁶ is —C(═O)C(═O)N(R²³)(R²⁴); R²³ is H; and R²⁴ is optionally substituted C₁-C₆heteroalkyl. In a further embodiment of the aforementioned embodiments of Formula (I) is a compound wherein R⁶ is —C(═O)C(═O)N(R²³)(R²⁴); R²³ is H; and R²⁴ is optionally substituted C₃-C₈cycloalkyl. In a further embodiment of the aforementioned embodiments of Formula (I) is a compound wherein R⁶ is —C(═O)C(═O)N(R²³)(R²⁴); R²³ is H; and R²⁴ is optionally substituted aralkyl. In a further embodiment of the aforementioned embodiments of Formula (I) is a compound wherein R⁶ is —C(═O)C(═O)N(R²³)(R²⁴); R²³ is H; and R²⁴ is optionally substituted aryl. In a further embodiment of the aforementioned embodiments of Formula (I) is a compound wherein R⁶ is —C(═O)C(═O)N(R²³)(R²⁴); R²³ is H; and R²⁴ is optionally substituted heteroaryl. In a further embodiment of the aforementioned embodiments of Formula (I) is a compound wherein R⁶ is —C(═O)C(═O)N(R²³)(R²⁴); R²³ is H; and R²⁴ is —CH₂C(O)OR²⁶. In a further embodiment of the aforementioned embodiments of Formula (I) is a compound wherein R⁶ is —C(═O)C(═O)N(R²³)(R²⁴); R²³ is H; and R²⁴ is —CH₂C(O)OR²⁶; and R²⁶ is optionally substituted C₁-C₆alkyl. In a further embodiment of the aforementioned embodiments of Formula (I) is a compound wherein R⁶ is —C(═O)C(═O)N(R²³)(R²⁴); R²³ is H; and R²⁴ is —CH₂CH₂R²⁷. In another embodiment of the aforementioned embodiments of Formula (I) is a compound wherein R⁶ is

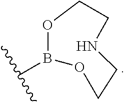 [SMILES: *B1OCCNCCO1]

In a further embodiment of the aforementioned embodiments of Formula (I) is a compound wherein n is 0.

In one embodiment is a compound of Formula (I) having the structure of Formula (I′):

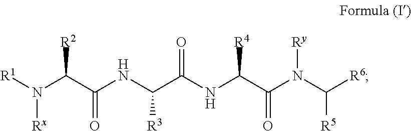

wherein:
R¹ is selected from:

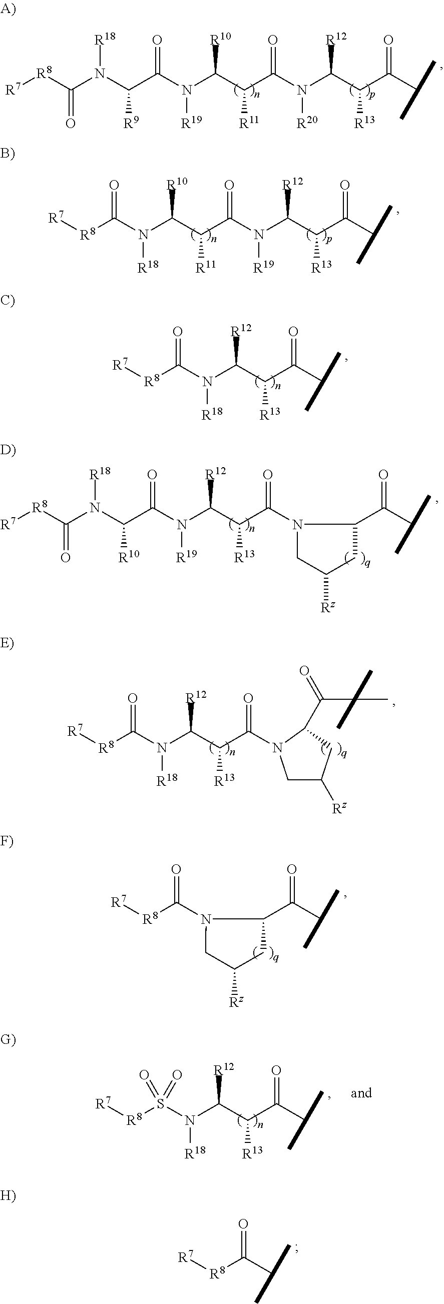

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

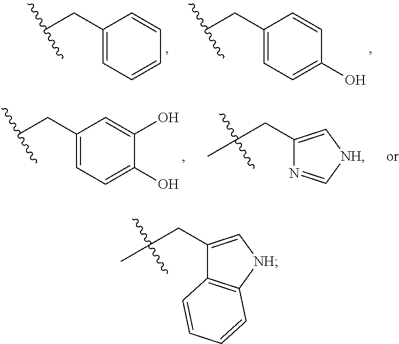

R³ is methyl, ethyl, isopropyl, or cyclopropyl;
R⁵ is H, methyl, ethyl, or —CH₂OH;
R⁶ is —C(═O)C(═O)N(R²³)(R²⁴), or

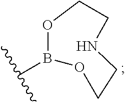 [SMILES: *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

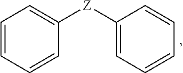

wherein Z is a bond, O, S, NH, CH₂, NHCH₂, or C≡C;
R⁸ is a bond, —O—, or —N(R¹⁷)—, optionally substituted heterocycloalkyl, optionally substituted aryl, or optionally substituted heteroaryl;
R⁹ is —CH₂OH, —CH₂CH(CH₃)₂,

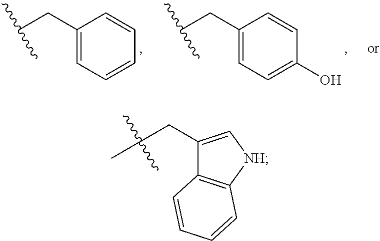

R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ is H, or C₁-C₄alkyl;
R²⁴ is optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²⁵ is independently C₁-C₆alkyl;
R²⁶ is H, or optionally substituted C₁-C₆alkyl;
R²⁷ is optionally substituted heterocycloalkyl, optionally substituted aryl, or optionally substituted heteroaryl;
n is 0 or 1;
p is 0 or 1; and
q is 0 or 1;
or a pharmaceutically acceptable salt, solvate, or prodrug thereof

In one embodiment is a compound of Formula (I) or Formula (I′) wherein R¹ is

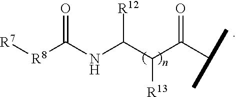

In a further embodiment, R⁸ is a bond. In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

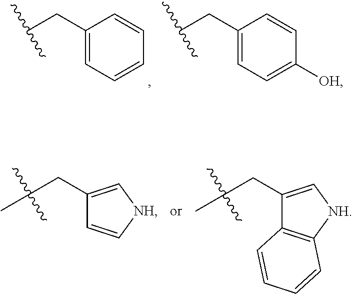

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

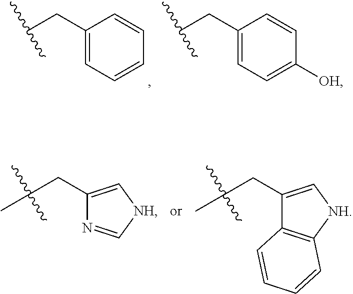

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

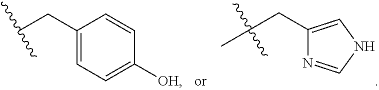 [SMILES: *Cc1ccc(O)cc1 ; *Cc1c[nH]cn1]

In a further embodiment of the aforementioned embodiments is a compound of Formula (I) or Formula (I′) wherein n is 0. In yet a further embodiment, n is 1.

In a further embodiment is a compound of Formula (I′) having the structure of Formula (Ia):

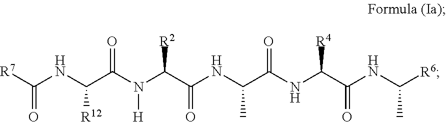

wherein R², R⁴, and R¹² are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (Ia) wherein R⁴ is —(CH₂)₄NH₂, R² is —CH(OH)(CH₃), and R¹² is —(CH₂)₂NH₂. In another embodiment is a compound of Formula (Ia) wherein R⁴ is —(CH₂)₄NH₂, R² is —CH(OH)(CH₃), and R¹² is —CH₂NH₂. In another embodiment is a compound of Formula (Ia) wherein R⁴ is —CH₂C(O)NH₂, R² is —CH(OH)(CH₃), and R¹² is —(CH₂)₄NH₂. In another embodiment is a compound of Formula (Ia) wherein R⁴ is —(CH₂)₄NH₂, R² is —(CH₂)₄NH₂, and R¹² is —CH₂NH₂. In another embodiment is a compound of Formula (Ia) wherein R⁴ is —CH₂C(O)NH₂, R² is —(CH₂)₄NH₂, and R¹² is —CH₂NH₂. In another embodiment is a compound of Formula (Ia) wherein R⁴ is —CH₂CH(CH₃)₂, R² is —(CH₂)₂NH₂, and R¹² is —(CH₂)₂NH₂. In another embodiment of the aforementioned embodiments of Formula (Ia) is a compound wherein R⁶ is —C(═O)C(═O)N(R²³)(R²⁴). In another embodiment of the aforementioned embodiments of Formula (Ia) is a compound wherein R⁶ is —C(═O)C(═O)N(H)(R²⁴). In yet a further embodiment of the aforementioned embodiments is a compound of Formula (Ia) wherein R⁷ is a linear or branched alkyl chain of 1-22 carbon atoms, optionally comprising within the alkyl chain or at an alkyl chain terminus an optionally substituted aryl, an optionally substituted heteroaryl, an optionally substituted heterocycloalkyl, or an optionally substituted

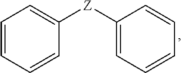

wherein Z is a bond, O, S, NH, CH₂, NHCH₂, or C≡C. In a further embodiment of the aforementioned embodiments is a compound of Formula (Ia) wherein R⁷ is a linear or branched alkyl chain of 1-22 carbon atoms, optionally comprising within the alkyl chain or at an alkyl chain terminus an optionally substituted

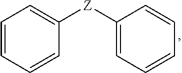

wherein Z is a bond.

In another embodiment is a compound of Formula (Ia) wherein R⁶ is

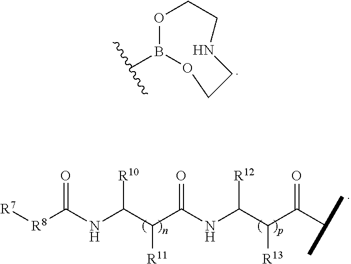

In another embodiment is a compound of Formula (I) or Formula (I′) wherein R¹ is

In a further embodiment, R⁸ is a bond. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

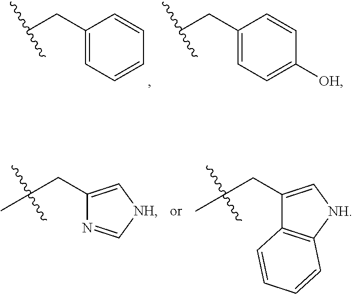

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

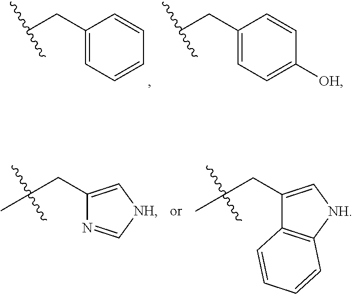

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂, or

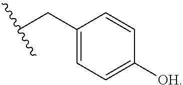 [SMILES: *Cc1ccc(O)cc1]

In a further embodiment of the aforementioned embodiments is a compound of Formula (I) or Formula (I′) wherein n is 0 and p is 0. In another embodiment, n is 0 and p is 1. In yet a further embodiment, n is 1 and p is 0.

In a further embodiment is a compound of Formula (I′) having the structure of Formula (Ib):

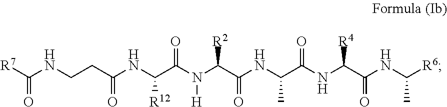

wherein R², R⁴, and R¹², are each independently —CH₂CH(CH₃)₂, —(CH₂)₃NH₂, or —(CH₂)₄NH₂.

In another embodiment is a compound of Formula (Ib) wherein R², R⁴, and R¹² are each —(CH₂)₄NH₂. In another embodiment is a compound of Formula (Ib) wherein R², R⁴, and R¹² are each —(CH₂)₃NH₂. In another embodiment is a compound of Formula (Ib) wherein R⁴ is —CH₂CH(CH₃)₂, R² is —(CH₂)₃NH₂, and R¹² is —(CH₂)₄NH₂. In another embodiment is a compound of Formula (Ib) wherein R⁴ is —CH₂CH(CH₃)₂, R² is —(CH₂)₄NH₂, and R¹² is —(CH₂)₄NH₂.

In a further embodiment is a compound of Formula (I′) having the structure of Formula (Ibb):

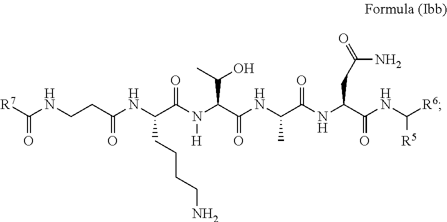

wherein R⁵ is —H, or —CH₃.

In a further embodiment is a compound of Formula (I′) having the structure of Formula (Ibbb):

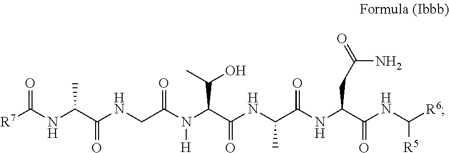

wherein R⁵ is —H, or —CH₃.

In another embodiment is a compound of Formula (I) or Formula (I′) wherein R¹ is

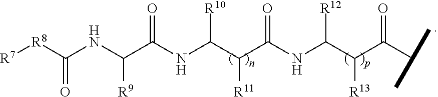

In a further embodiment, R⁸ is a bond. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, (CH₂)₄NH₂,

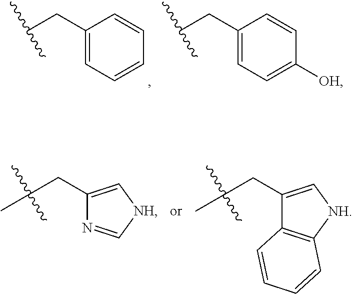

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

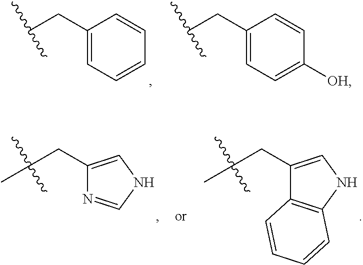

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

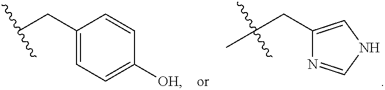 [SMILES: *Cc1ccc(O)cc1 ; *Cc1c[nH]cn1]

In a further embodiment of the aforementioned embodiments is a compound of Formula (I) or Formula (I′) wherein n is 0 and p is 0. In another embodiment, n is 0 and p is 1. In yet a further embodiment, n is 1 and p is 0.

In a further embodiment is a compound of Formula (I′) having the structure of Formula (Ic):

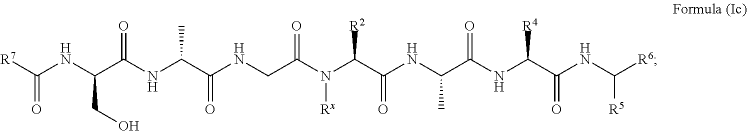

wherein R², R⁴, and R¹², are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (Ic) wherein R⁴ is —(CH₂)₄NH₂, R² is —CH(OH)(CH₃), and R¹² is —(CH₂)₂NH₂. In another embodiment is a compound of Formula (Ic) wherein R⁴ is —(CH₂)₄NH₂, R² is —CH(OH)(CH₃), and R¹² is —CH₂NH₂. In another embodiment is a compound of Formula (Ic) wherein R⁴ is —CH₂C(O)NH₂, R² is —CH(OH)(CH₃), and R¹² is —(CH₂)₄NH₂. In another embodiment is a compound of Formula (Ic) wherein R⁴ is —(CH₂)₄NH₂, R² is —(CH₂)₄NH₂, and R¹² is —CH₂NH₂. In another embodiment is a compound of Formula (Ic) wherein R⁴ is —CH₂C(O)NH₂, R² is —(CH₂)₄NH₂, and R¹² is —CH₂NH₂. In another embodiment is a compound of Formula (Ic) wherein R⁴ is —CH₂CH(CH₃)₂, R² is —(CH₂)₂NH₂, and R¹² is —(CH₂)₂NH₂.

In a further embodiment is a compound of Formula (I′) having the structure of Formula (Icc):

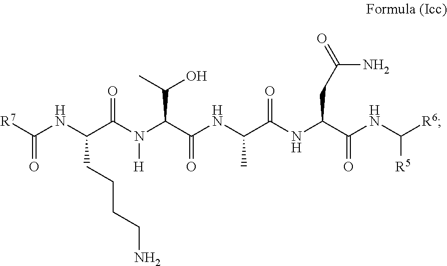

wherein R⁵ is —H, or —CH₃.

In another embodiment is a compound of Formula (I) or Formula (I′) wherein R¹ is

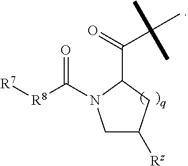

In a further embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

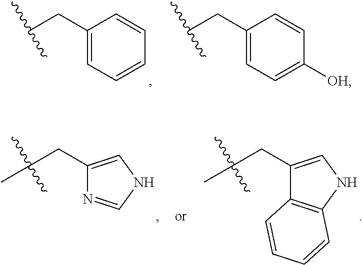

In a further embodiment, q is 1 and R⁸ is a bond.

In a further embodiment is a compound of Formula (I′) having the structure of Formula (Id):

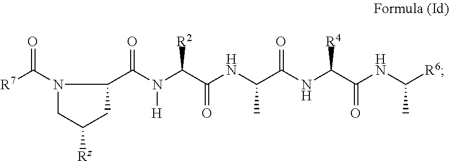

wherein R^{z} is NH₂; and R² and R⁴ are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (Id) wherein R² is —CH(OH)(CH₃), and R⁴ is —CH₂C(O)NH₂. In another embodiment is a compound of Formula (Id) wherein R² is —CH(OH)(CH₃), and R⁴ is —(CH₂)₂NH₂. In another embodiment is a compound of Formula (Id) wherein R² is —CH(OH)(CH₃), and R⁴ is —(CH₂)₃NH₂. In another embodiment is a compound of Formula (Id) wherein R² is —CH(OH)(CH₃), and R⁴ is —(CH₂)₄NH₂. In another embodiment is a compound of Formula (Id) wherein R² is —(CH₂)₄NH₂ and R⁴ is —CH₂CH(CH₃)₂. In another embodiment is a compound of Formula (Id) wherein R² is —(CH₂)₄NH₂ and R⁴ is —CH₂C(O)NH₂. In another embodiment is a compound of Formula (Id) wherein R² is —(CH₂)₄NH₂ and R⁴ is —(CH₂)₄NH₂.

In another embodiment is a compound of Formula (I) or Formula (I′) wherein R¹ is

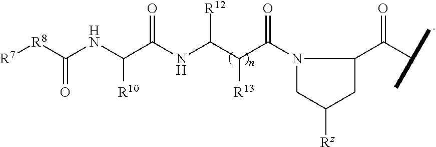

In a further embodiment, R⁸ is a bond. In another embodiment, R², R⁴, R¹⁰, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

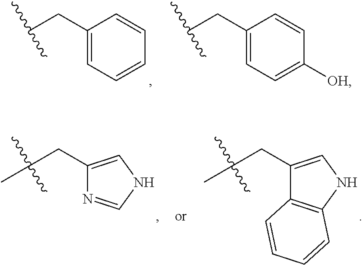

In a further embodiment, R², R⁴, R¹⁰, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

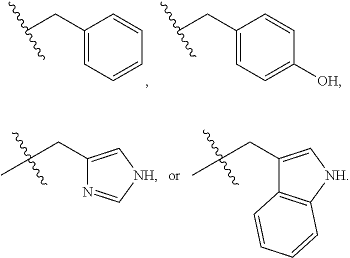

In yet a further embodiment, R², R⁴, R¹⁰, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

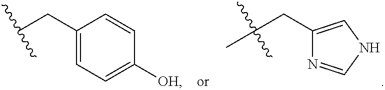 [SMILES: *Cc1ccc(O)cc1 ; *Cc1c[nH]cn1]

In a further embodiment of the aforementioned embodiments is a compound of Formula (I) or Formula (I′) wherein n is 0. In yet a further embodiment, n is 1.

In a further embodiment is a compound of Formula (I′) having the structure of Formula (Idd):

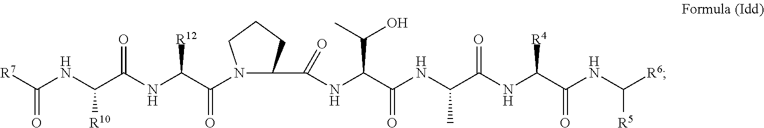

wherein R⁵ is —H, or —CH₃.

In another embodiment is a compound of Formula (Idd) wherein R¹⁰ is —CH₂OH, and R¹² is —CH₃. In another embodiment is a compound of Formula (Idd) wherein R¹⁰ is —CH₂CH(CH₃)₂, and R¹² is —CH(OH)(CH₃). In another embodiment of the aforementioned compounds of Formula (Id) is a compound wherein R⁴ is —CH₂C(O)NH₂. In yet another embodiment of the aforementioned compounds of Formula (Idd) is a compound wherein R⁴ is

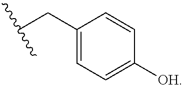 [SMILES: *Cc1ccc(O)cc1]

In another embodiment is a compound of Formula (I) or Formula (I′) wherein R¹ is

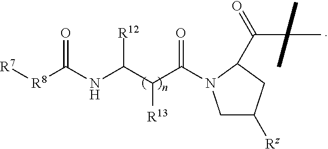

In a further embodiment, R⁸ is a bond. In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

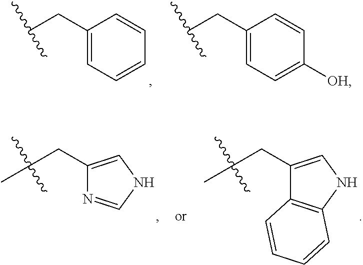

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

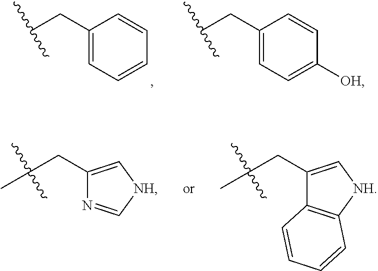 [SMILES: *Cc1ccccc1 ; *Cc1ccc(O)cc1 ; *Cc1c[nH]cn1 ; *Cc1c[nH]c2ccccc12]

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂NH₂, —CH₂CH₂C(O)NH₂, —(CH₂)₂NH₂, —(CH₂)₃NH₂, —(CH₂)₄NH₂,

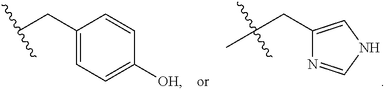 [SMILES: *Cc1ccc(O)cc1 ; *Cc1c[nH]cn1]

In a further embodiment of the aforementioned embodiments is a compound of Formula (I) or Formula (I′) wherein n is 0. In yet a further embodiment, n is 1.

In another embodiment is a compound of Formula (I) or Formula (I′) wherein R¹ is

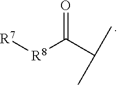

In a further embodiment, R⁸ is a bond. In another embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

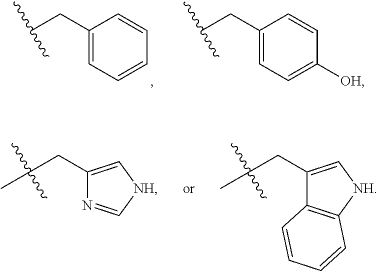 [SMILES: *Cc1ccccc1 ; *Cc1ccc(O)cc1 ; *Cc1c[nH]cn1 ; *Cc1c[nH]c2ccccc12]

In a further embodiment, R² and R⁴ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

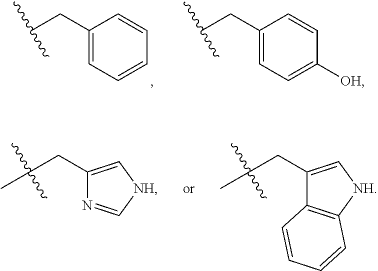 [SMILES: *Cc1ccccc1 ; *Cc1ccc(O)cc1 ; *Cc1c[nH]cn1 ; *Cc1c[nH]c2ccccc12]

In yet a further embodiment, R² and R⁴ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

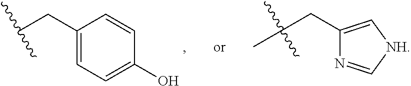 [SMILES: *Cc1ccc(O)cc1 ; *Cc1c[nH]cn1]

In another embodiment is a compound of Formula (I) or Formula (I′) wherein R^{x} and R² together with the nitrogen atom form an optionally substituted nitrogen containing ring. In a further embodiment is a compound of Formula (I′) having the structure of Formula (Ie):

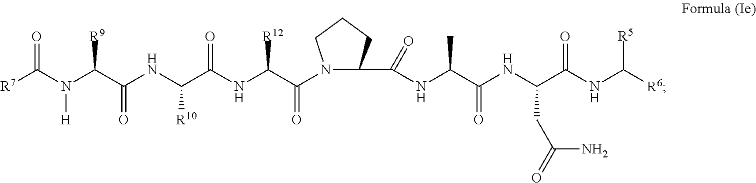

wherein R⁵ is —H, or —CH₃.

In another embodiment is a compound of Formula (Ie) wherein R¹⁰ and R¹² are each independently —H, —CH₃, —CH₂CH(CH₃)₂, —CH₂OH, or —CH(OH)(CH₃).

In another embodiment of any of the aforementioned embodiments of Formula (I) or Formula (I′) is a compound wherein R⁶ is —C(═O)H.

In another embodiment described herein are compounds of Formula (II):

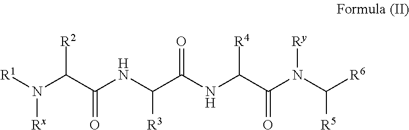

wherein:
R¹ is selected from:

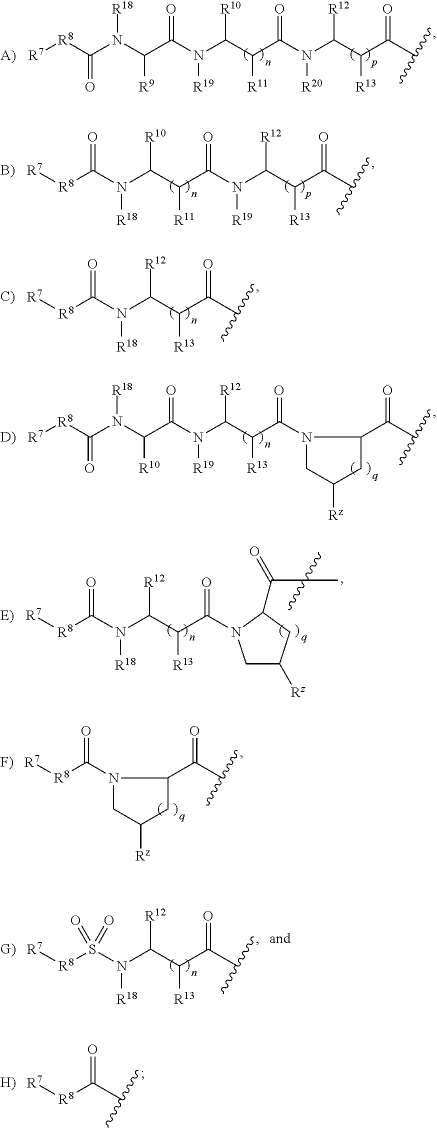

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

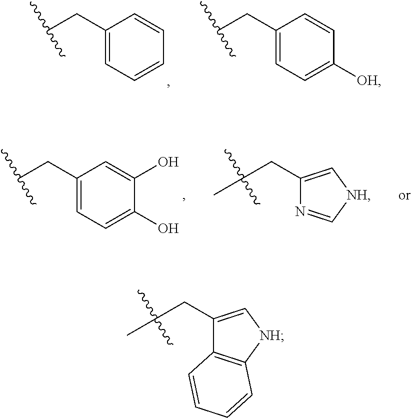 [SMILES: *Cc1ccccc1 ; *Cc1ccc(O)cc1 ; *Cc1ccc(O)c(O)c1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —C(═O)H, —CH₂C(═O)H, —C(═O)NHCH₂C(═O)H, —C(═O)C(═O)N(R¹⁴)₂, —B(OR²³)(OR²⁴), or

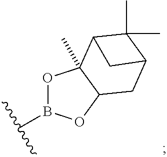 [SMILES: *B1OC2CC3CC(C3(C)C)[C@]2(C)O1]

or R⁵ and R⁶ together with the carbon atom form

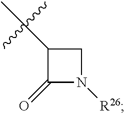

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

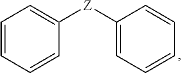

wherein Z is a bond, O, S, NH, CH₂, NHCH₂, or C≡C;
R⁸ is an optionally substituted heterocycloalkyl;
R⁹ is —CH₂OH, —CH₂CH(CH₃)₂,

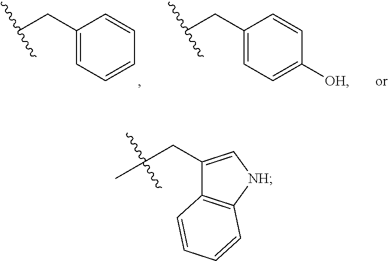 [SMILES: *Cc1ccccc1 ; *Cc1ccc(O)cc1 ; "or" ; *Cc1c[nH]c2ccccc12]

R¹⁴, R¹⁵, and R¹⁶ are each independently H, or C₁-C₄alkyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ and R²⁴ are each independently H, or C₁-C₄alkyl; or R²³ and R²⁴ together with the boron atom form an optionally substituted 5- or 6-membered boron containing ring;
each R²⁵ is independently C₁-C₆alkyl;
R²⁶ is H, C₁-C₄alkyl, C₁-C₄alkoxy, —CH₂C(O)OR²⁵, or —OCH₂C(O)OR²⁵;
n is 0 or 1;
p is 0 or 1; and
q is 0 or 1;
or a pharmaceutically acceptable salt, solvate, or prodrug thereof

In another embodiment is a compound of Formula (II) having the structure of Formula (II′):

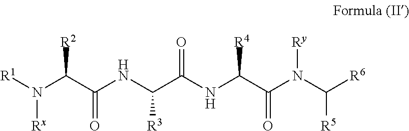

wherein:
R¹ is selected from:

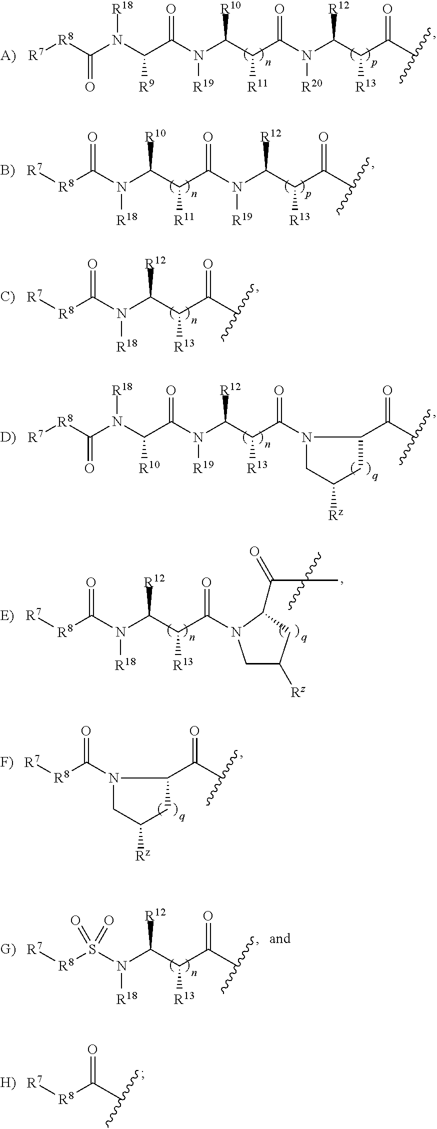

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

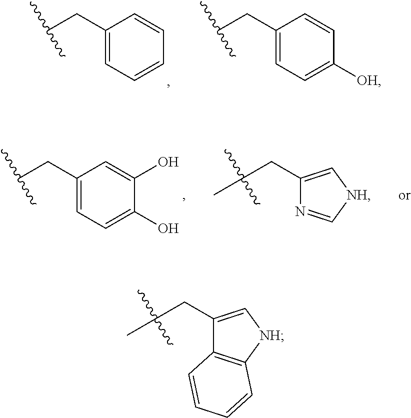 [SMILES: *Cc1ccccc1 ; *Cc1ccc(O)cc1 ; *Cc1ccc(O)c(O)c1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —C(═O)H, —CH₂C(═O)H, —C(═O)NHCH₂C(═O)H, —C(═O)C(═O)N(R¹⁴)₂, —B(OR²³)(OR²⁴), or

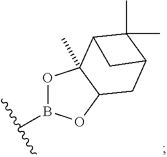 [SMILES: *B1OC2CC3CC(C3(C)C)[C@]2(C)O1]

or R⁵ and R⁶ together with the carbon atom form

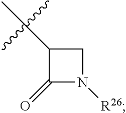

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶; —CH₂—NR¹⁵R¹⁶; or —(CH₂)₂—NR¹⁵R¹⁶;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

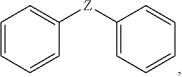

wherein Z is a bond, O, S, NH, CH₂, NHCH₂, or C≡C;
R⁸ is an optionally substituted heterocycloalkyl;
R⁹ is —CH₂OH, —CH₂CH(CH₃)₂,

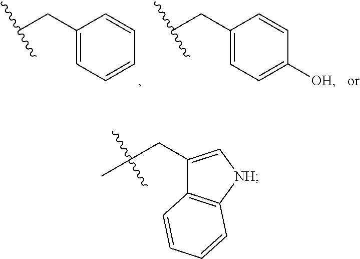

R¹⁴, R¹⁵, and R¹⁶ are each independently H, or C₁-C₄alkyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ and R²⁴ are each independently H, or C₁-C₄alkyl; or R²³ and R²⁴ together with the boron atom form an optionally substituted 5- or 6-membered boron containing ring;
each R²⁵ is independently C₁-C₆alkyl;
R²⁶ is H, C₁-C₄alkyl, C₁-C₄alkoxy, —CH₂C(O)OR²⁵, or —OCH₂C(O)OR²⁵;
n is 0 or 1;
p is 0 or 1; and
q is 0 or 1;
or a pharmaceutically acceptable salt, solvate, or prodrug thereof

In one embodiment is a compound of Formula (II) or Formula (II′) wherein R¹ is

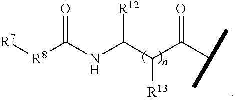

In another embodiment is a compound of Formula (II) or Formula (II′) wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, (CH₂)₄NH₂.

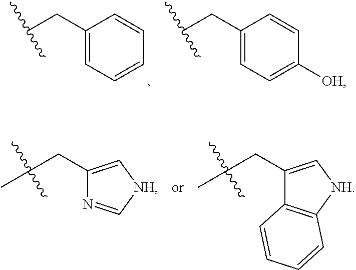

In a further embodiment is a compound of Formula (II) or Formula (II′) wherein R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

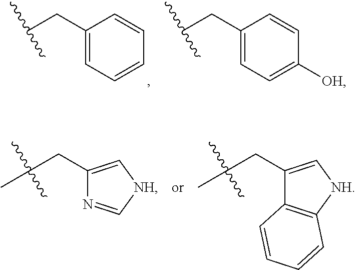

In yet a further embodiment is a compound of Formula (II) or Formula (II′) wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, (CH₂)₄NH₂,

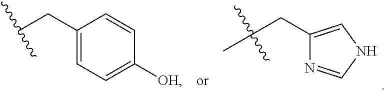 [SMILES: *Cc1ccc(O)cc1 ; *Cc1c[nH]cn1 ; "or"]

In a further embodiment of the aforementioned embodiments of Formula (II) or Formula (II′) is a compound wherein n is 0. In another further embodiment of the aforementioned embodiments of Formula (II) or Formula (II′) is a compound wherein n is 1. In another embodiment of the aforementioned embodiments of Formula (II) or (II′) is a compound wherein R⁸ is piperidine and R⁷ is optionally substituted aryl.

In another embodiment described herein are compounds of Formula (III):

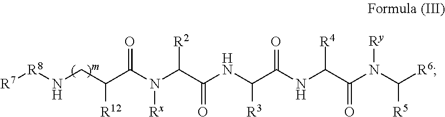

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

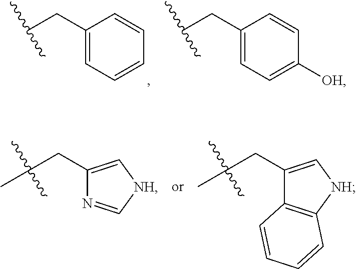

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴),

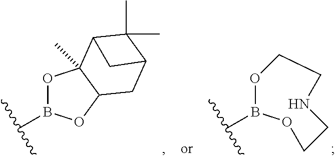 [SMILES: *B1OC2CC3CC(C3(C)C)[C@]2(C)O1 ; *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

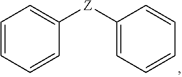

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is —NR²¹R²²;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 0 or 1;
or a pharmaceutically acceptable salt, solvate, or prodrug thereof

In another embodiment is a compound of Formula (III) having the structure of Formula (III′):

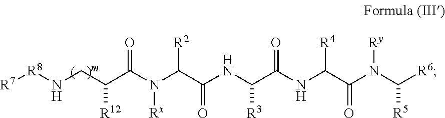

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

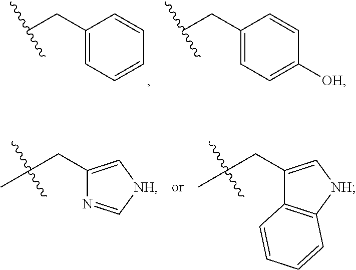

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴),

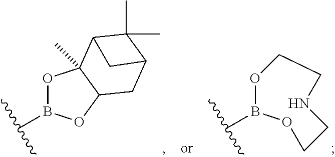 [SMILES: *B1OC2CC3CC(C3(C)C)[C@]2(C)O1 ; *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

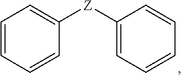

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is —NR²¹R²²;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 0 or 1; or a pharmaceutically acceptable salt, solvate, or prodrug thereof.

In some embodiments is a compound of Formula (III) or Formula (III′) wherein R⁸ is a bond. In another embodiment is a compound of Formula (III) or Formula (III′) wherein R⁸ is an optionally substituted C₁-C₆alkyl. In another embodiment is a compound of Formula (III) or Formula (III′) wherein R⁸ is an optionally substituted C₁-C₆heteroalkyl.

In a further embodiment is a compound of Formula (III) or Formula (III′) wherein R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²². In yet a further embodiment is a compound of Formula (III) or Formula (III′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment is a compound of Formula (III) or Formula (III′) wherein m is 1, and R¹² is —NR²¹R²². In another embodiment is a compound of Formula (III) or Formula (III′) wherein m is 1, and R¹² is —NH₂.

In another embodiment described herein are compounds of Formula (IV):

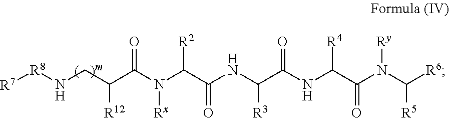

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

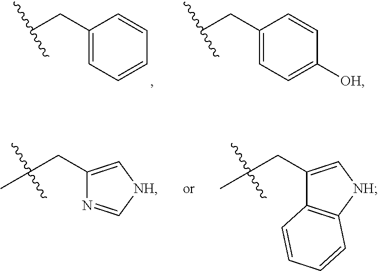 [SMILES: *Cc1ccccc1 ; *Cc1ccc(O)cc1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴),

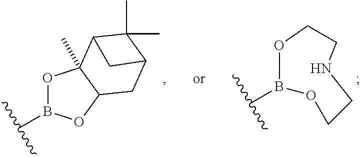 [SMILES: *B1OC2CC3CC(C3(C)C)[C@]2(C)O1 ; *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

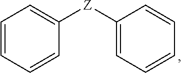

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is H;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 0;
or a pharmaceutically acceptable salt, solvate, or prodrug thereof

In another embodiment is a compound of Formula (IV) having the structure of Formula (IV′):

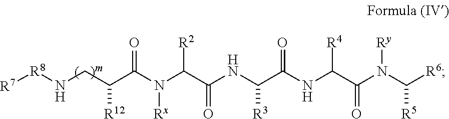

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

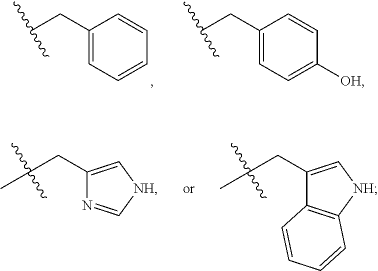 [SMILES: *Cc1ccccc1 ; *Cc1ccc(O)cc1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴),

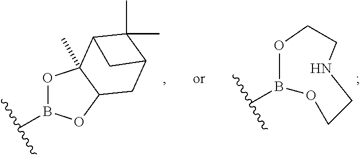 [SMILES: *B1OC2CC3CC(C3(C)C)[C@]2(C)O1 ; *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

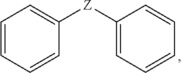

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is H;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 0; or a pharmaceutically acceptable salt, solvate, or prodrug thereof

In some embodiments is a compound of Formula (IV) or Formula (IV′) wherein R⁸ is a bond. In another embodiment is a compound of Formula (IV) or Formula (IV′) wherein R⁸ is an optionally substituted C₁-C₆alkyl. In another embodiment is a compound of Formula (IV) or Formula (IV′) wherein R⁸ is an optionally substituted C₁-C₆heteroalkyl.

In a further embodiment is a compound of Formula (IV) or Formula (IV′) wherein R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²². In yet a further embodiment is a compound of Formula (IV) or Formula (IV′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment described herein are compounds of Formula (V):

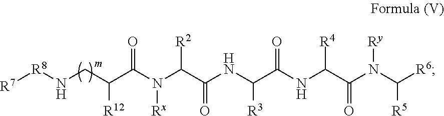

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

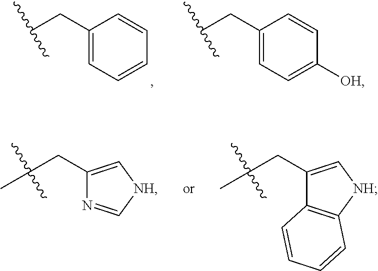 [SMILES: *Cc1ccccc1 ; *Cc1ccc(O)cc1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴), or

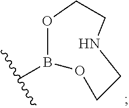 [SMILES: *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

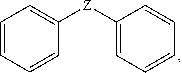

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is H or —NR²¹R²²;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 2-4;
or a pharmaceutically acceptable salt, solvate, or prodrug thereof

In another embodiment is a compound of Formula (V) having the structure of Formula (V′):

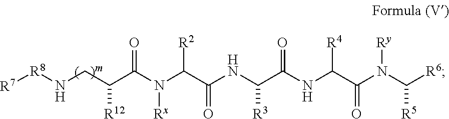

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

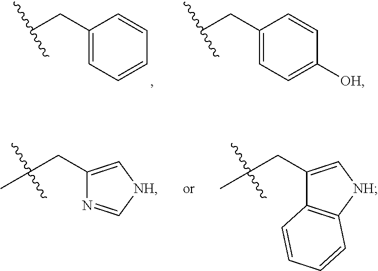 [SMILES: *Cc1ccccc1 ; *Cc1ccc(O)cc1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴), or

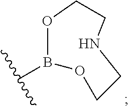 [SMILES: *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

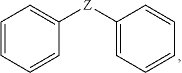

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is H or —NR²¹R²²;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 2-4; or a pharmaceutically acceptable salt, solvate, or prodrug thereof

In some embodiments is a compound of Formula (V) or Formula (V′) wherein R⁸ is a bond. In another embodiment is a compound of Formula (V) or Formula (V′) wherein R⁸ is an optionally substituted C₁-C₆alkyl. In another embodiment is a compound of Formula (V) or Formula (V′) wherein R⁸ is an optionally substituted C₁-C₆heteroalkyl.

In a further embodiment is a compound of Formula (V) or Formula (V′) wherein R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²². In yet a further embodiment is a compound of Formula (V) or Formula (V′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment is a compound of Formula (V) or Formula (V′) wherein R¹² is H. In another embodiment is a compound of Formula (V) or Formula (V′) wherein m is 2, and R¹² is H. In another embodiment is a compound of Formula (V) or Formula (V′) wherein m is 3, and R¹² is H. In another embodiment is a compound of Formula (V) or Formula (V′) wherein m is 4, and R¹² is H. In another embodiment is a compound of (V) or Formula (V′) wherein m is 2, and R¹² is —NR²¹R²². In another embodiment is a compound of Formula (V) or Formula (V′) wherein m is 2, and R¹² is —NH₂. In another embodiment is a compound of (V) or Formula (V′) wherein m is 3, and R¹² is —NR²¹R²². In another embodiment is a compound of Formula (V) or Formula (V′) wherein m is 3, and R¹² is —NH₂. In another embodiment is a compound of Formula (V) or Formula (V′) wherein m is 4, and R¹² is —NR²¹R²². In another embodiment is a compound of Formula (V) or Formula (V′) wherein m is 4, and R¹² is —NH₂.

In another embodiment described herein are compounds of Formula (VI):

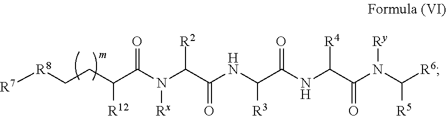

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

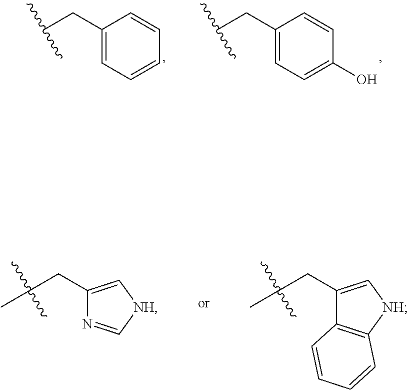 [SMILES: *Cc1ccccc1 ; *Cc1ccc(O)cc1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴),

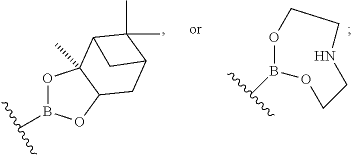 [SMILES: *B1OC2CC3CC(C3(C)C)[C@]2(C)O1 ; *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

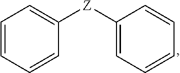

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is —NR²¹R²²;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 0-4;
or a pharmaceutically acceptable salt, solvate, or prodrug thereof

In another embodiment is a compound of Formula (VI) having the structure of Formula (VI′):

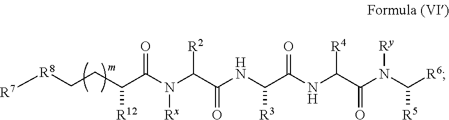

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

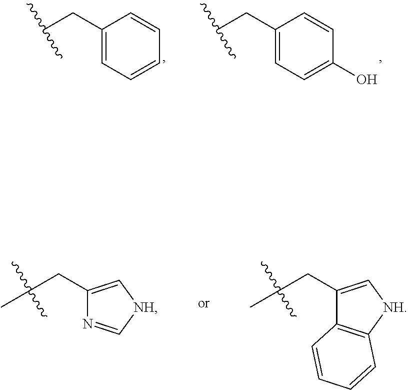 [SMILES: *Cc1ccccc1 ; *Cc1ccc(O)cc1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴),

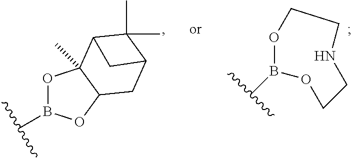 [SMILES: *B1OC2CC3CC(C3(C)C)[C@]2(C)O1 ; *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

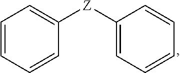

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is —NR²¹R²²;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 0-4; or a pharmaceutically acceptable salt, solvate, or prodrug thereof

In some embodiments is a compound of Formula (VI) or Formula (VI′) wherein R⁸ is a bond. In another embodiment is a compound of Formula (VI) or Formula (VI′) wherein R⁸ is an optionally substituted C₁-C₆alkyl. In another embodiment is a compound of Formula (VI) or Formula (VI′) wherein R⁸ is an optionally substituted C₁-C₆heteroalkyl.

In a further embodiment is a compound of Formula (VI) or Formula (VI′) wherein R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²². In yet a further embodiment is a compound of Formula (VI) or Formula (VI′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment is a compound of Formula (VI) or Formula (VI′) wherein m is 0, and R¹² is —NH₂. In another embodiment is a compound of (VI) or Formula (VI′) wherein m is 1, and R¹² is —NH₂. In another embodiment is a compound of Formula (VI) or Formula (VI′) wherein m is 2, and R¹² is —NH₂. In another embodiment is a compound of Formula (VI) or Formula (VI′) wherein m is 3, and R¹² is —NH₂. In another embodiment is a compound of Formula (VI) or Formula (VI′) wherein m is 4, and R¹² is —NH₂.

In another embodiment described herein are compounds of Formula (VII):

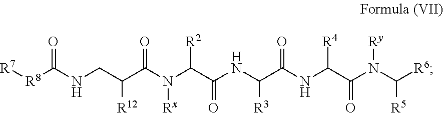

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

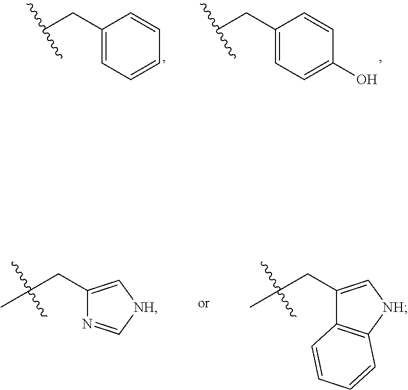 [SMILES: *Cc1ccccc1 ; *Cc1ccc(O)cc1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴),

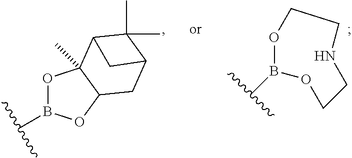 [SMILES: *B1OC2CC3CC(C3(C)C)[C@]2(C)O1 ; *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

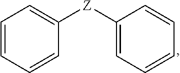

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is —NR²¹R²²;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and or a pharmaceutically acceptable salt, solvate, or prodrug thereof

In another embodiment is a compound of Formula (VII) having the structure of Formula (VII′):

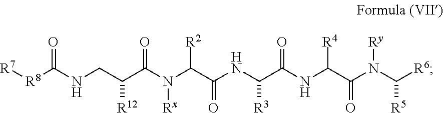

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

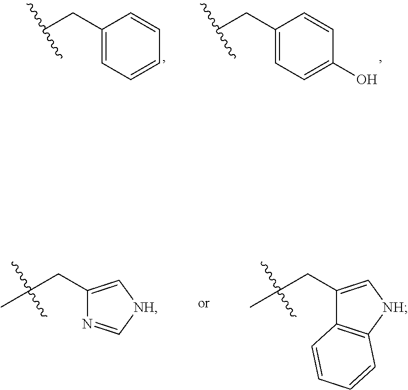 [SMILES: *Cc1ccccc1 ; *Cc1ccc(O)cc1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —CH₂C(═O)H, —C(═O)NHCH₂C(═O)H, —C(═O)C(═O)N(R¹⁴)₂, —B(OR²³)(OR²⁴),

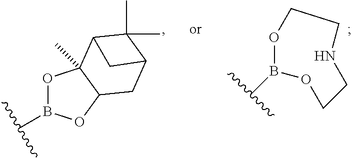 [SMILES: *B1OC2CC3CC(C3(C)C)[C@]2(C)O1 ; *B1OCCNCCO1]

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

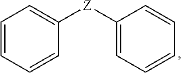

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹² is —NR²¹R²²;
each R¹⁴ is independently H, optionally substituted C₁-C₄alkyl, optionally substituted C₁-C₆heteroalkyl, optionally substituted C₃-C₈cycloalkyl, optionally substituted heterocycloalkyl, optionally substituted aralkyl, optionally substituted aryl, optionally substituted heteroaryl, —CH₂C(O)OR²⁶, or —CH₂CH₂R²⁷;
each R²¹ is independently H, or C₁-C₄alkyl;
each R²² is independently H, C₁-C₄alkyl, —C(O)R²⁸, —C(═NH)(NH₂), or —CH(═NH);
R²³ and R²⁴ are each independently H, or C₁-C₄alkyl;
R²⁶ is H, or optionally substituted C₁-C₆alkyl;
R²⁷ is optionally substituted heterocycloalkyl, optionally substituted aryl, or optionally substituted heteroaryl;
R²⁸ is H, or optionally substituted C₁-C₆alkyl; and
m is 0-4; or a pharmaceutically acceptable salt, solvate, or prodrug thereof

In some embodiments is a compound of Formula (VII) or Formula (VII′) wherein R⁸ is a bond. In another embodiment is a compound of Formula (VII) or Formula (VII′) wherein R⁸ is an optionally substituted C₁-C₆alkyl. In another embodiment is a compound of Formula (VII) or Formula (VII′) wherein R⁸ is an optionally substituted C₁-C₆heteroalkyl.

In a further embodiment is a compound of Formula (VII) or Formula (VII′) wherein R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²². In yet a further embodiment is a compound of Formula (VII) or Formula (VII′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment is a compound of Formula (VII) or Formula (VII′) wherein R¹² is —NH₂.

In another embodiment of the aforementioned embodiments of Formula (I), (II), (III), (IV), (V), (VI), or (VII) is a compound wherein R⁷ is a linear or branched alkyl chain of about 1-22 carbon atoms. In another embodiment of the aforementioned embodiments of Formula (I), (II), (III), (IV), (V), (VI), or (VII) is a compound wherein R⁷ is

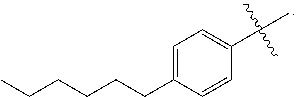 [SMILES: *c1ccc(CCCCCC)cc1]

In another embodiment of the aforementioned embodiments of Formula (I), (II), (III), (IV), (V), (VI), or (VII) is a compound wherein R⁷ is

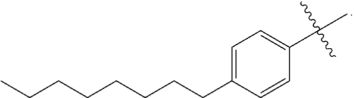 [SMILES: *c1ccc(CCCCCCCC)cc1]

In another embodiment of the aforementioned embodiments of Formula (I), (II), (III), (IV), (V), (VI), or (VII) is a compound wherein R⁷ is

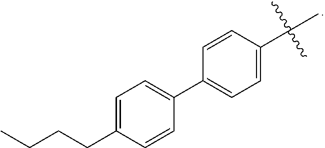 [SMILES: *c1ccc(-c2ccc(CCCC)cc2)cc1]

In another embodiment of the aforementioned embodiments of Formula (I), (II), (III), (IV), (V), (VI), or (VII) is a compound wherein R⁵ is H. In another embodiment of the aforementioned embodiments of Formula (I), (II), (III), (IV), (V), (VI), or (VII) is a compound wherein R⁵ is methyl. In another embodiment of the aforementioned embodiments of Formula (I), (II), (III), (IV), (V), (VI), or (VII) is a compound wherein R⁵ is —CH₂OH. In another embodiment of the aforementioned embodiments of Formula (I), (II), (III), (IV), (V), (VI), or (VII) is a compound wherein R⁶ is —B(OH)₂. In another embodiment of the aforementioned embodiments of Formula (I), (II), (III), (IV), (V), (VI), or (VII) is a compound wherein R⁶ is —B(OR²³)(OR²⁴). In another embodiment of the aforementioned embodiments of Formula (I), (II), (III), (IV), (VI), or (VII) is a compound wherein R⁶ is

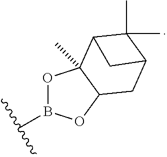 [SMILES: *B1OC2CC3CC(C3(C)C)[C@]2(C)O1]

In another embodiment of the aforementioned embodiments of Formula (I), (III), (IV), (VI), or (VII) is a compound wherein R⁵ is methyl, R⁶ is

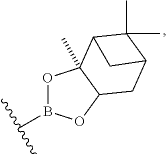 [SMILES: *B1OC2CC3CC(C3(C)C)[C@]2(C)O1]

R⁸ is a bond, and R⁷ is

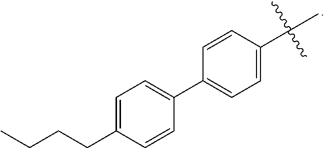 [SMILES: *c1ccc(-c2ccc(CCCC)cc2)cc1]

In another embodiment of the aforementioned embodiments of Formula (I), (III), (IV), (V), (VI), or (VII) is a compound wherein R⁵ is methyl, R⁶ is —B(OH)₂, R⁸ is a bond, and R⁷ is

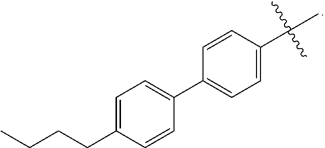 [SMILES: *c1ccc(-c2ccc(CCCC)cc2)cc1]

In another embodiment of the aforementioned embodiments of Formula (I), (III), (IV), (VI), or (VII) is a compound wherein R⁵ is methyl, R⁶ is

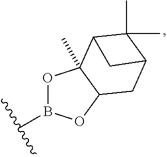 [SMILES: *B1OC2CC3CC(C3(C)C)[C@]2(C)O1]

R⁸ is a bond, and R⁷ is

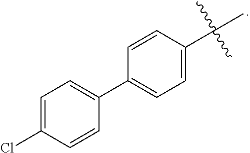 [SMILES: *c1ccc(-c2ccc(Cl)cc2)cc1]

In another embodiment of the aforementioned embodiments of Formula (I), (III), (IV), (V), (VI), or (VII) is a compound wherein R⁵ is methyl, R⁶ is —B(OH)₂, R⁸ is a bond, and R⁷ is

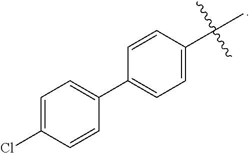 [SMILES: *c1ccc(-c2ccc(Cl)cc2)cc1]

In another embodiment of the aforementioned embodiments of Formula (I), (III), (IV), (VI), or (VII) is a compound wherein R⁵ is methyl, R⁶ is

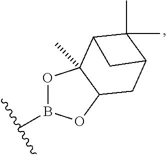 [SMILES: *B1OC2CC3CC(C3(C)C)[C@]2(C)O1]

R⁸ is a bond, and R⁷ is

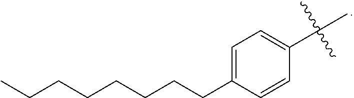 [SMILES: *c1ccc(CCCCCCCC)cc1]

In another embodiment of the aforementioned embodiments of Formula (I), (III), (IV), (V), (VI), or (VII) is a compound wherein R⁵ is methyl, R⁶ is —B(OH)₂, R⁸ is a bond, and R⁷ is

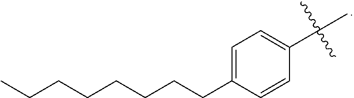 [SMILES: *c1ccc(CCCCCCCC)cc1]

In another embodiment of the aforementioned embodiments of Formula (I), (III), (IV), (VI), or (VII) is a compound wherein R⁵ is methyl, R⁶ is

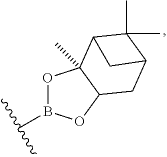 [SMILES: *B1OC2CC3CC(C3(C)C)[C@]2(C)O1]

R⁸ is heteroaryl, and R⁷ is

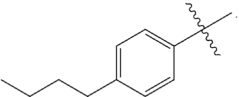 [SMILES: *c1ccc(CCCC)cc1]

In another embodiment of the aforementioned embodiments of Formula (I), (III), (IV), (V), (VI), or (VII) is a compound wherein R⁵ is methyl, R⁶ is —B(OH)₂, R⁸ is heteroaryl, and R⁷ is

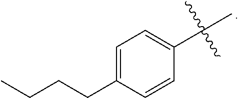 [SMILES: *c1ccc(CCCC)cc1]

In another embodiment of the aforementioned embodiments of Formula (I), (II), (III), (IV), (VI), or (VII) is a compound wherein R⁵ is methyl, R⁶ is

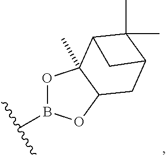 [SMILES: *B1OC2CC3CC(C3(C)C)[C@]2(C)O1]

R⁸ is heterocycloalkyl, and R⁷ is

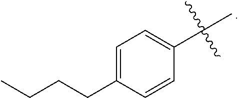 [SMILES: *c1ccc(CCCC)cc1]

In another embodiment of the aforementioned embodiments of Formula (I), (II), (III), (IV), (V), (VI), or (VII) is a compound wherein R⁵ is methyl, R⁶ is —B(OH)₂, R⁸ is heterocycloalkyl, and R⁷ is

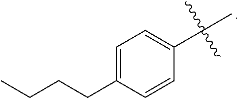 [SMILES: *c1ccc(CCCC)cc1]

In another embodiment of the aforementioned embodiments of Formula (I′), (II′), (III′), (IV′), (V′), (VI′), or (VII′) is a compound wherein R⁷ is a linear or branched alkyl chain of about 1-22 carbon atoms. In another embodiment of the aforementioned embodiments of Formula (I′), (II′), (III′), (IV′), (V′), (VI′), or (VII′) is a compound wherein R⁷ is

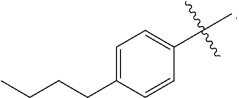 [SMILES: *c1ccc(CCCC)cc1]

In another embodiment of the aforementioned embodiments of Formula (I′), (II′), (III′), (IV′), (V′), (VI′), or (VII′) is a compound wherein R⁷ is

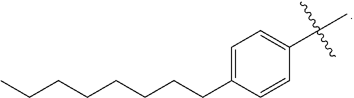 [SMILES: *c1ccc(CCCCCCCC)cc1]

In another embodiment of the aforementioned embodiments of Formula (I′), (II′), (III′), (IV′), (V′), (VI′), or (VII′) is a compound wherein R⁷ is

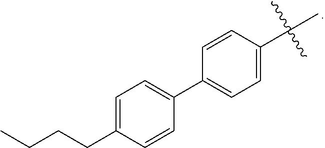 [SMILES: *c1ccc(-c2ccc(CCCC)cc2)cc1]

In another embodiment of the aforementioned embodiments of Formula (I′), (II′), (III′), (IV′), (V′), (VI′), or (VII′) is a compound wherein R⁵ is H. In another embodiment of the aforementioned embodiments of Formula (I′), (II′), (III′), (IV′), (V′), (VI′), or (VII′) is a compound wherein R⁵ is methyl. In another embodiment of the aforementioned embodiments of (I′), (II′), (III′), (IV′), (V′), (VI′), or (VII′) is a compound wherein R⁵ is —CH₂OH. In another embodiment of the aforementioned embodiments of Formula (I′), (II′), (III′), (IV′), (V′), (VI′), or (VII′) is a compound wherein R⁶ is —B(OH)₂. In another embodiment of the aforementioned embodiments of Formula (I′), (II′), (III′), (IV′), (V′), (VI′), or (VII′) is a compound wherein R⁶ is —B(OR²³)(OR²⁴). In another embodiment of the aforementioned embodiments of Formula (I′), (II′), (III′), (IV′), (VI′), or (VII′) is a compound wherein R⁶ is

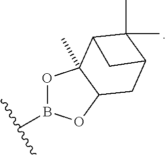 [SMILES: *B1OC2CC3CC(C3(C)C)[C@]2(C)O1]

In another embodiment of the aforementioned embodiments of Formula (I′), (III′), (IV′), (VI′), or (VII′) is a compound wherein R⁵ is methyl, R⁶ is

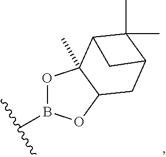 [SMILES: *B1OC2CC3CC(C3(C)C)[C@]2(C)O1]

R⁸ is a bond, and R⁷ is

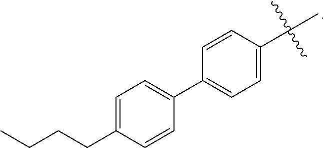 [SMILES: *c1ccc(-c2ccc(CCCC)cc2)cc1]

In another embodiment of the aforementioned embodiments of Formula (I′), (III′), (IV′), (V′), (VI′), or (VII′) is a compound wherein R⁵ is methyl, R⁶ is —B(OH)₂, R⁸ is a bond, and R⁷ is

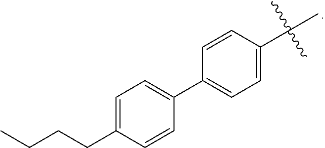 [SMILES: *c1ccc(-c2ccc(CCCC)cc2)cc1]

In another embodiment of the aforementioned embodiments of Formula (I′), (III′), (IV′), (VI′), or (VII′) is a compound wherein R⁵ is methyl, R⁶ is

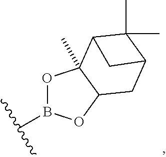 [SMILES: *B1OC2CC3CC(C3(C)C)[C@]2(C)O1]

R⁸ is a bond, and R⁷ is

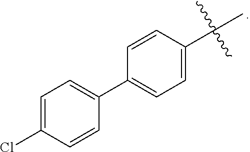 [SMILES: *c1ccc(-c2ccc(Cl)cc2)cc1]

In another embodiment of the aforementioned embodiments of Formula (I′), (III′), (IV′), (V′), (VI′), or (VII′) is a compound wherein R⁵ is methyl, R⁶ is —B(OH)₂, R⁸ is a bond, and R⁷ is

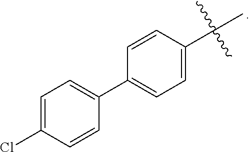 [SMILES: *c1ccc(-c2ccc(Cl)cc2)cc1]

In another embodiment of the aforementioned embodiments of Formula (I′), (III′), (IV′), (VI′), or (VII′) is a compound wherein R⁵ is methyl, R⁶ is

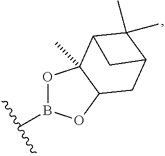 [SMILES: *B1OC2CC3CC(C3(C)C)[C@]2(C)O1]

R⁸ is a bond, and R⁷ is

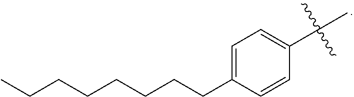 [SMILES: *c1ccc(CCCCCCCC)cc1]

In another embodiment of the aforementioned embodiments of Formula (I′), (III′), (IV′), (V′), (VI′), or (VII′) is a compound wherein R⁵ is methyl, R⁶ is —B(OH)₂, R⁸ is a bond, and R⁷ is

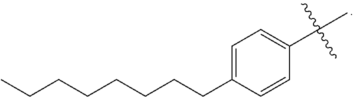 [SMILES: *c1ccc(CCCCCCCC)cc1]

In another embodiment of the aforementioned embodiments of Formula (I′), (III′), (IV′), (V′), (VI′), or (VII′) is a compound wherein R⁵ is methyl, R⁶ is

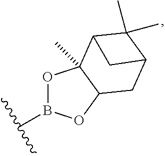 [SMILES: *B1OC2CC3CC(C3(C)C)[C@]2(C)O1]

R⁸ is heteroaryl, and R⁷ is

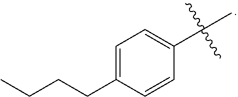 [SMILES: *c1ccc(CCCC)cc1]

In another embodiment of the aforementioned embodiments of Formula (I′), (III′), (IV′), (V′), (VI′), or (VII′) is a compound wherein R⁵ is methyl, R⁶ is —B(OH)₂, R⁸ is heteroaryl, and R⁷ is

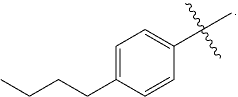 [SMILES: *c1ccc(CCCC)cc1]

In another embodiment of the aforementioned embodiments of Formula (I′), (II′), (III′), (IV′), (V′), (VI′), or (VII′) is a compound wherein R⁵ is methyl, R⁶ is

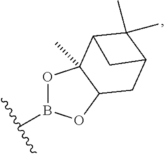 [SMILES: *B1OC2CC3CC(C3(C)C)[C@]2(C)O1]

R⁸ is heterocycloalkyl, and R⁷ is

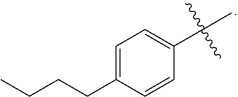 [SMILES: *c1ccc(CCCC)cc1]

In another embodiment of the aforementioned embodiments of Formula (I′), (II′), (III′), (IV′), (V′), (VI′), or (VII′) is a compound wherein R⁵ is methyl, R⁶ is —B(OH)₂, R⁸ is heterocycloalkyl, and R⁷ is

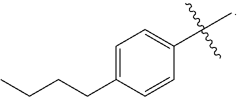 [SMILES: *c1ccc(CCCC)cc1]

In another aspect is a compound selected from:

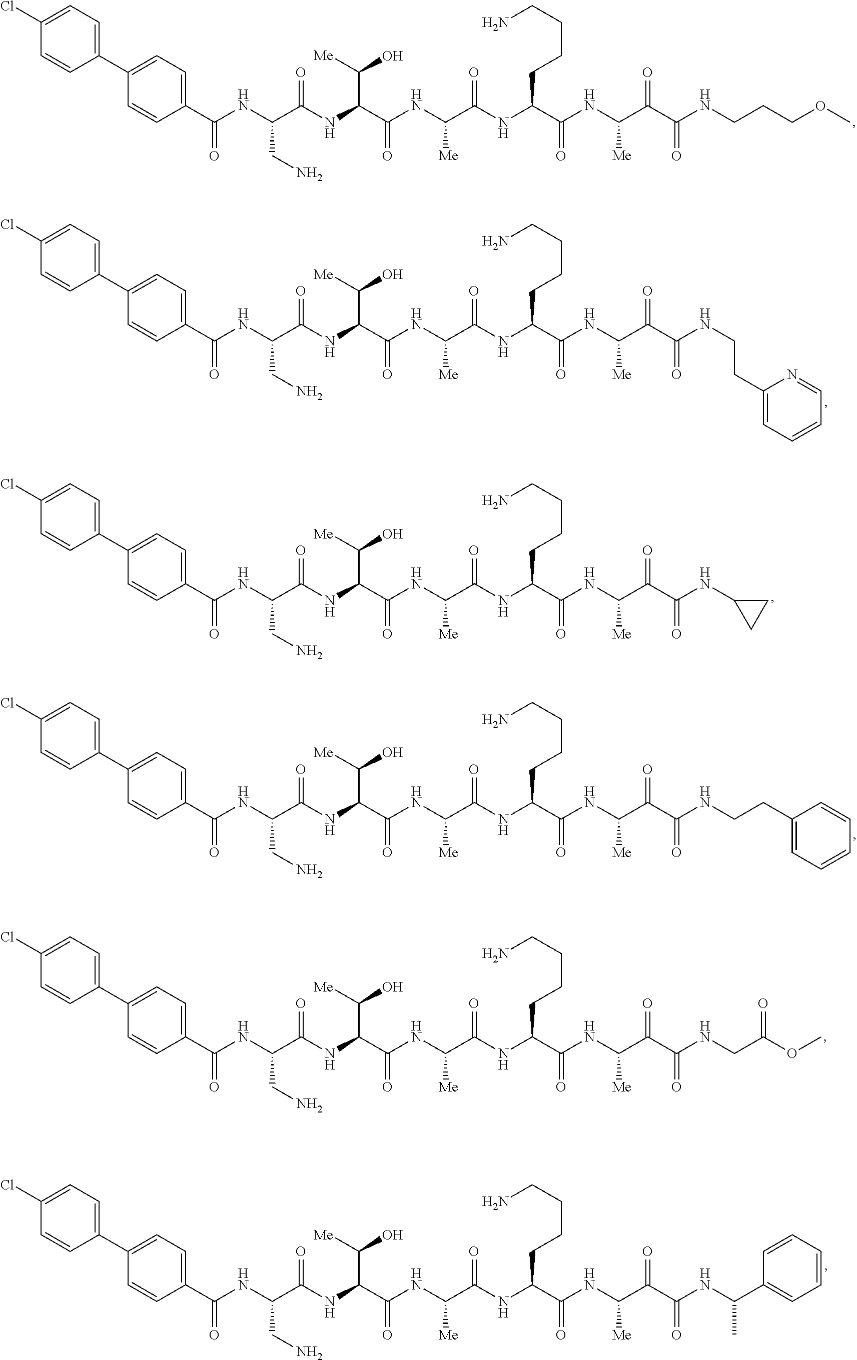

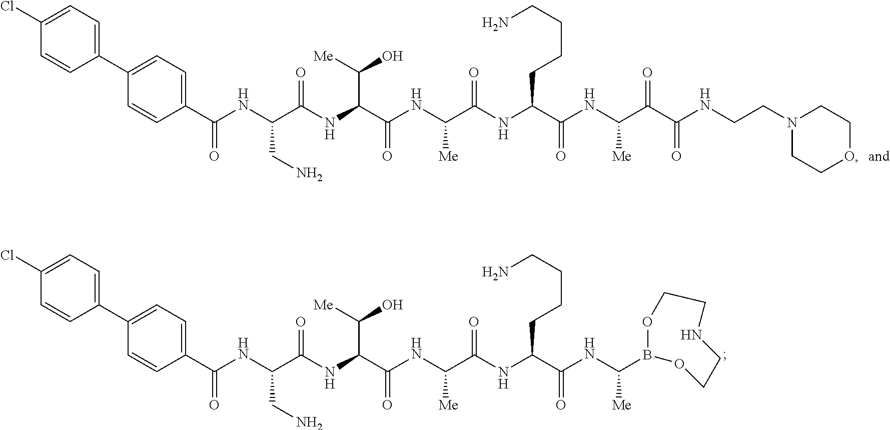 [SMILES: C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)C(=O)NCCN1CCOCC1 ; C[C@H](NC(=O)[C@H](CCCCN)NC(=O)[C@H](C)NC(=O)[C@@H](NC(=O)[C@H](CN)NC(=O)c1ccc(-c2ccc(Cl)cc2)cc1)[C@@H](C)O)B1OCCNCCO1]

or a pharmaceutically acceptable salt, solvate, or prodrug thereof.

In another embodiment is a compound selected from:

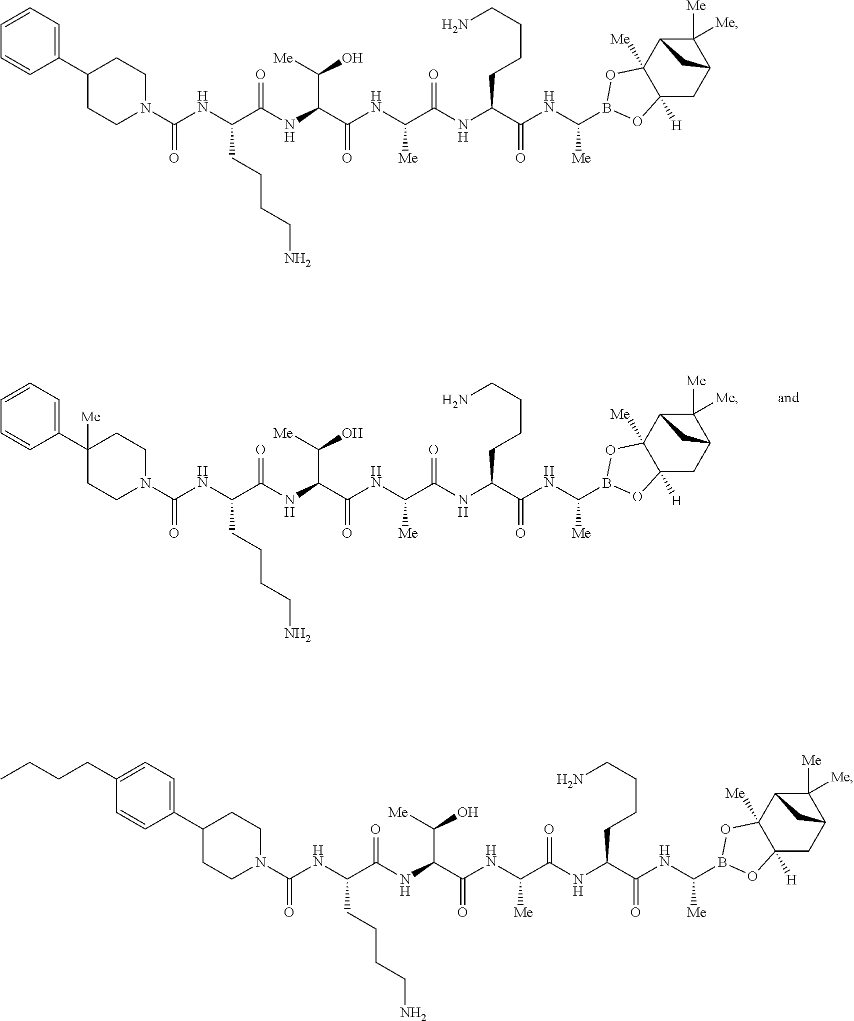 [SMILES: C[C@H](NC(=O)[C@H](CCCCN)NC(=O)[C@H](C)NC(=O)[C@@H](NC(=O)[C@H](CCCCN)NC(=O)N1CCC(c2ccccc2)CC1)[C@@H](C)O)B1O[C@@H]2C[C@@H]3C[C@@H](C3(C)C)[C@]2(C)O1 ; C[C@H](NC(=O)[C@H](CCCCN)NC(=O)[C@H](C)NC(=O)[C@@H](NC(=O)[C@H](CCCCN)NC(=O)N1CCC(C)(c2ccccc2)CC1)[C@@H](C)O)B1O[C@@H]2C[C@@H]3C[C@@H](C3(C)C)[C@]2(C)O1 ; "and" ; CCCCc1ccc(C2CCN(C(=O)N[C@@H](CCCCN)C(=O)N[C@H](C(=O)N[C@@H](C)C(=O)N[C@@H](CCCCN)C(=O)N[C@@H](C)B3O[C@@H]4C[C@@H]5C[C@@H](C5(C)C)[C@]4(C)O3)[C@@H](C)O)CC2)cc1]

or a pharmaceutically acceptable salt, solvate, or prodrug thereof.

In another embodiment is a compound selected from:

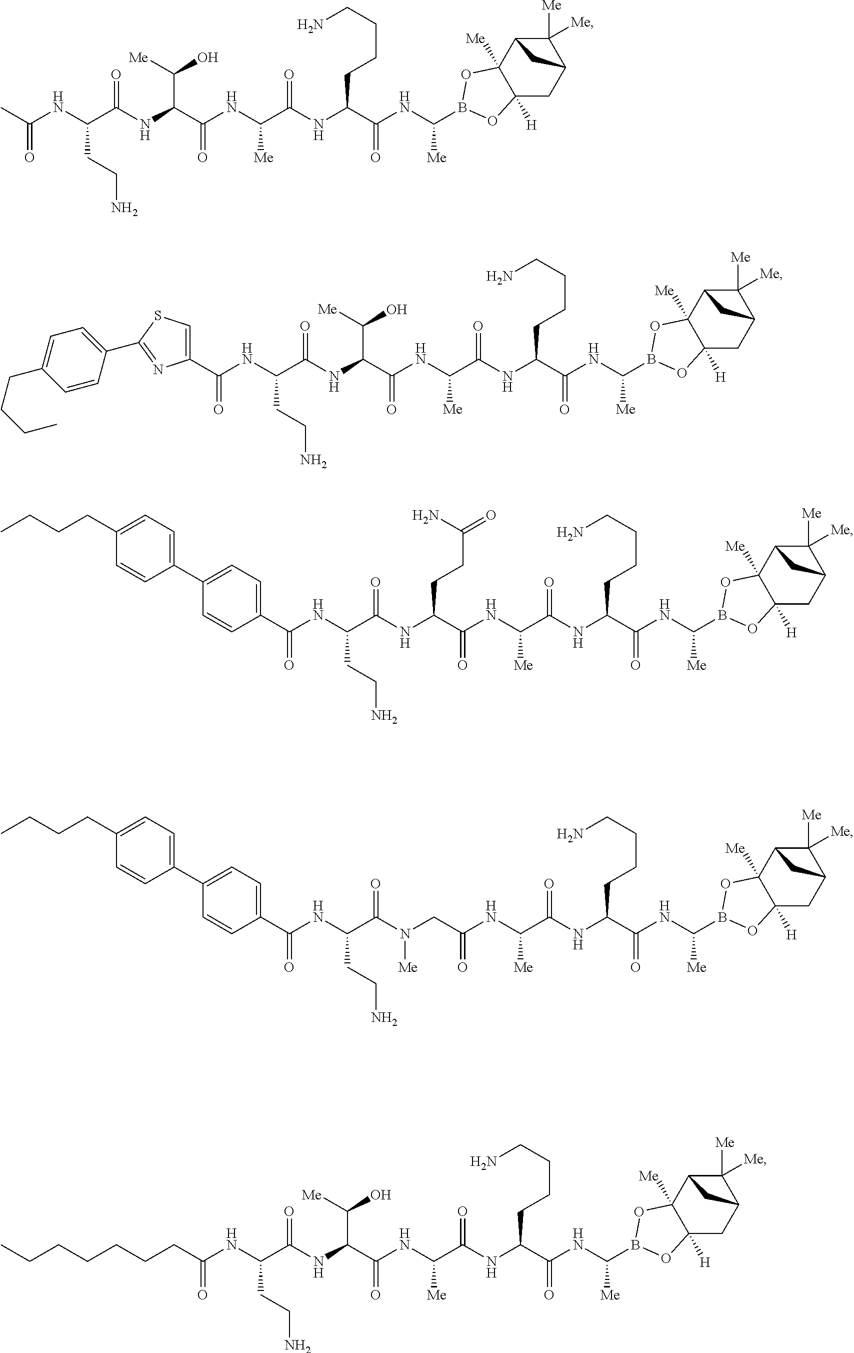

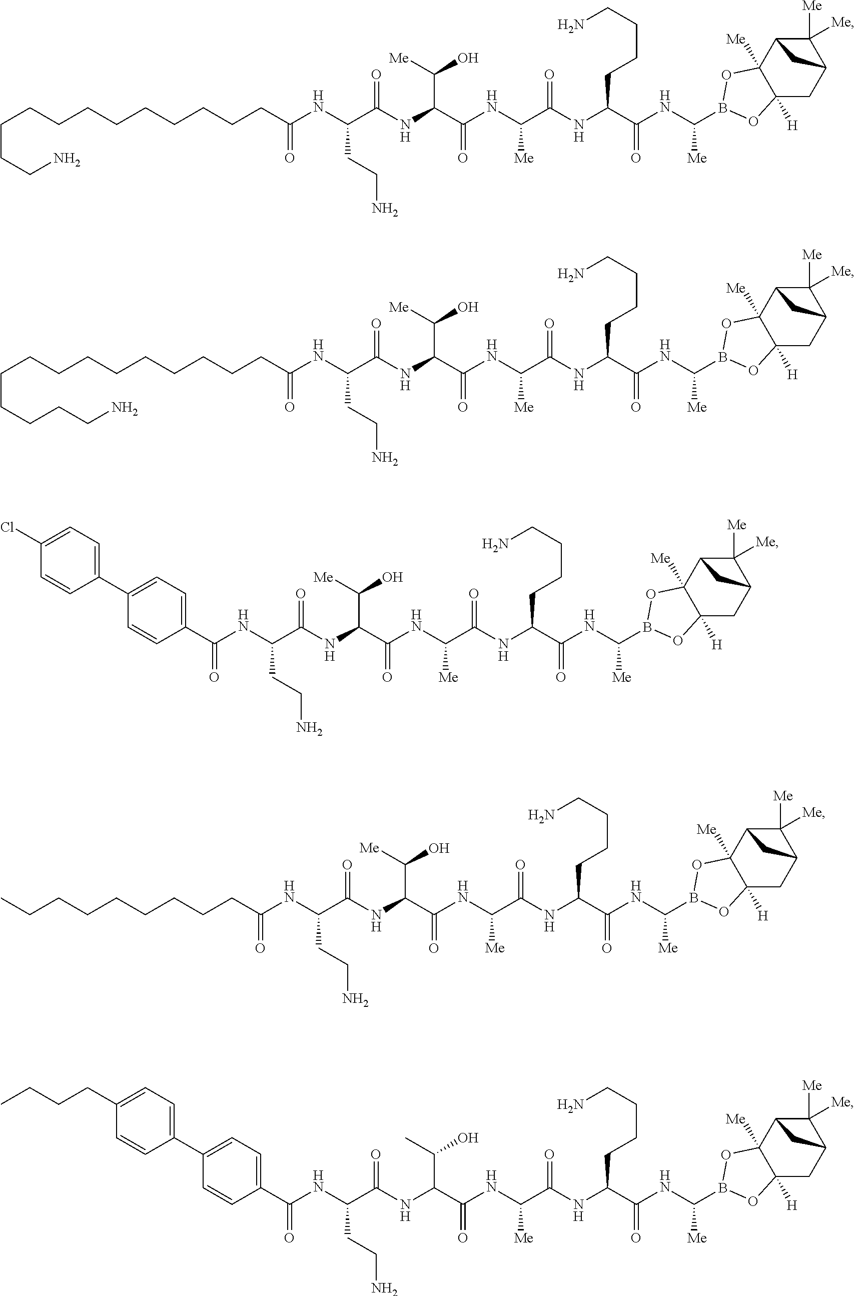

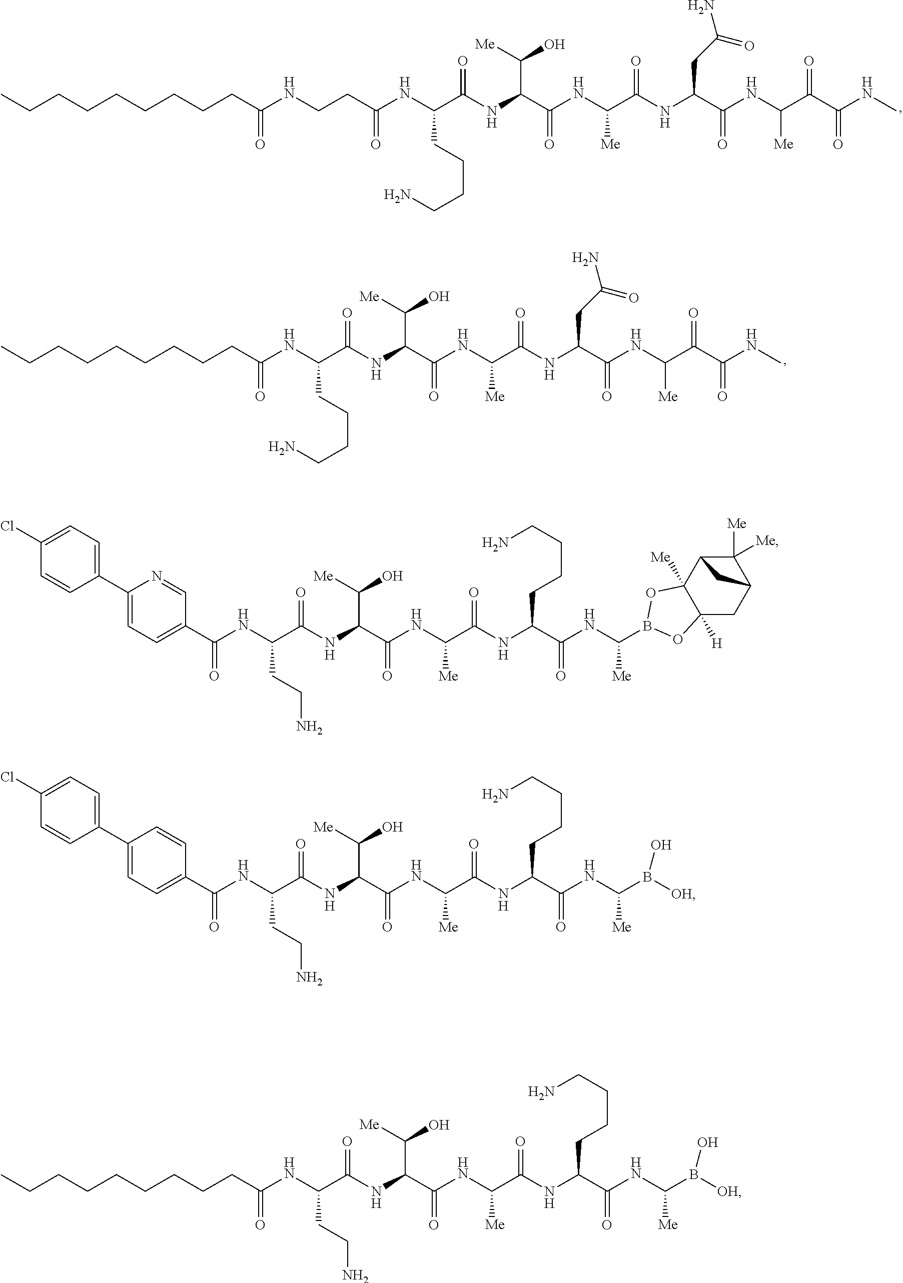

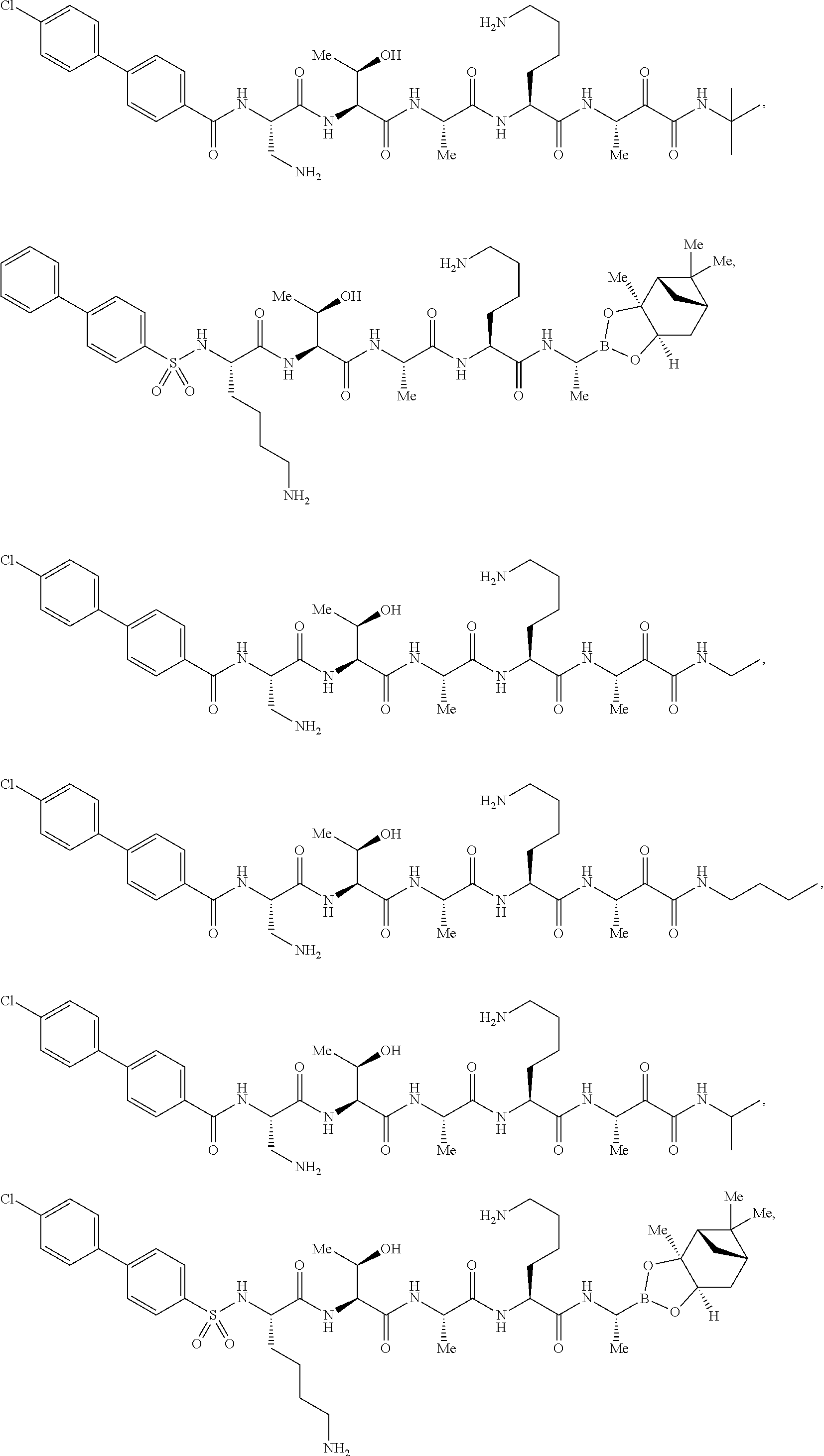

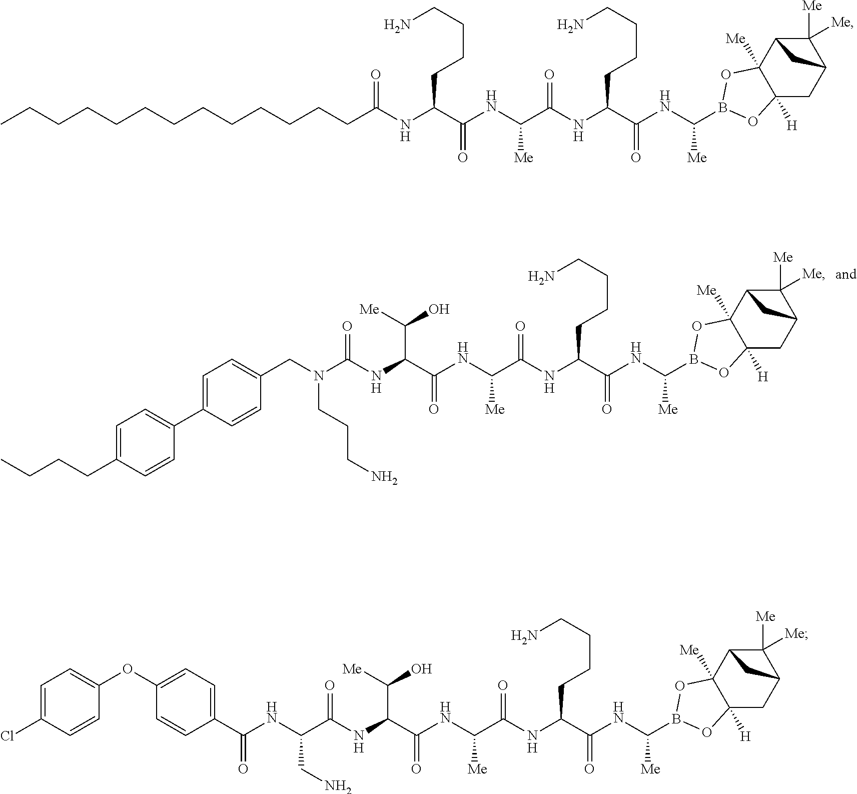 [SMILES: CCCCCCCCCCCCCC(=O)N[C@@H](CCCCN)C(=O)N[C@@H](C)C(=O)N[C@@H](CCCCN)C(=O)N[C@@H](C)B1O[C@@H]2C[C@@H]3C[C@@H](C3(C)C)[C@]2(C)O1 ; CCCCc1ccc(-c2ccc(CN(CCCN)C(=O)N[C@H](C(=O)N[C@@H](C)C(=O)N[C@@H](CCCCN)C(=O)N[C@@H](C)B3O[C@@H]4C[C@@H]5C[C@@H](C5(C)C)[C@]4(C)O3)[C@@H](C)O)cc2)cc1 ; C[C@H](NC(=O)[C@H](CCCCN)NC(=O)[C@H](C)NC(=O)[C@@H](NC(=O)[C@H](CN)NC(=O)c1ccc(Oc2ccc(Cl)cc2)cc1)[C@@H](C)O)B1O[C@@H]2C[C@@H]3C[C@@H](C3(C)C)[C@]2(C)O1]

or a pharmaceutically acceptable salt, solvate, or prodrug thereof.

In one aspect described herein are compounds of Formula (VIII):

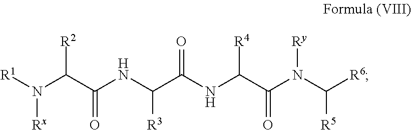

wherein:
R¹ is selected from:

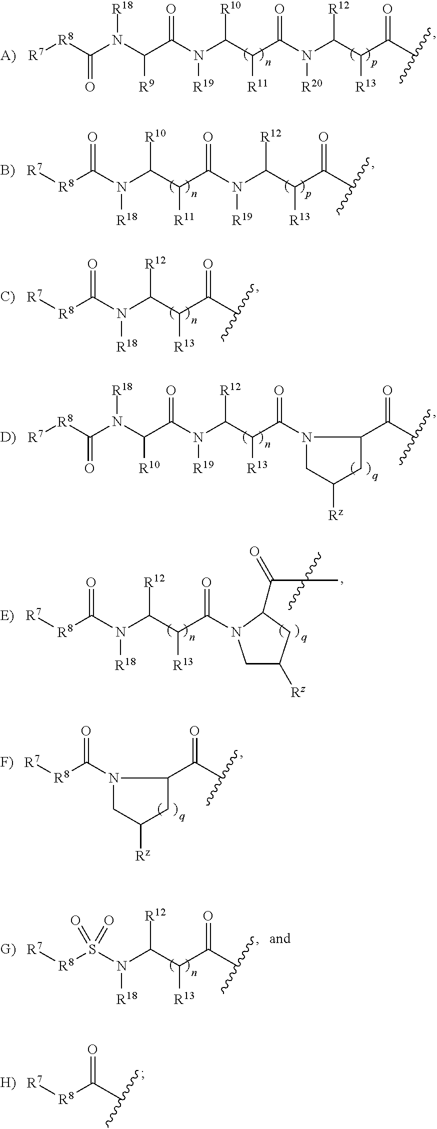

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

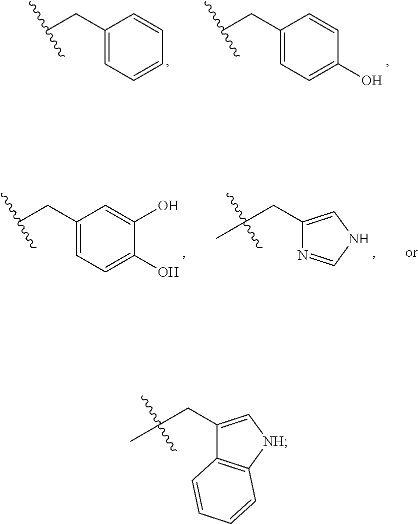 [SMILES: *Cc1ccccc1 ; *Cc1ccc(O)cc1 ; *Cc1ccc(O)c(O)c1 ; *Cc1c[nH]cn1 ; *Cc1c[nH]c2ccccc12]

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

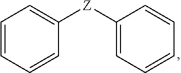

wherein Z is a bond, O, S, NH, CH₂, NHCH₂, or C≡C;
R⁸ is a bond, —O—, or —N(R¹⁷)—, optionally substituted C₁-C₆alkyl, optionally substituted C₁-C₆heteroalkyl, optionally substituted heterocycloalkyl, optionally substituted aryl, or optionally substituted heteroaryl;
R⁹ is —CH₂OH, —CH₂CH(CH₃)₂,

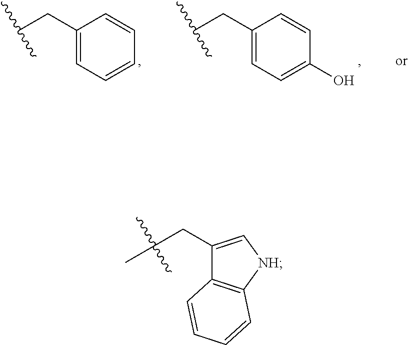 [SMILES: *Cc1ccccc1 ; *Cc1ccc(O)cc1 ; *Cc1c[nH]c2ccccc12]

R¹⁴ is C₁-C₆alkyl, C₁-C₆haloalkyl, or

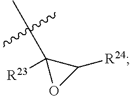

R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

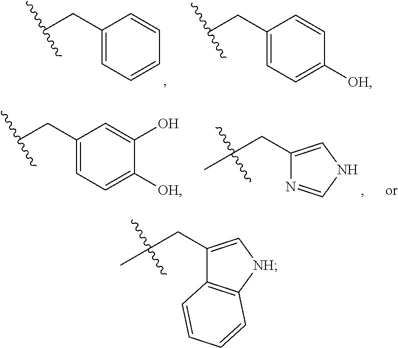

each R²⁵ is independently C₁-C₆alkyl;
R²⁶ is H, or C₁-C₄alkyl;
R²⁷ is H, or C₁-C₄alkyl;
n is 0 or 1;
p is 0 or 1; and
q is 0 or 1;
or a pharmaceutically acceptable salt, solvate, or prodrug thereof.

In one embodiment is a compound of Formula (VIII) having the structure of Formula (VIII′):

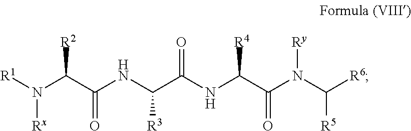

wherein:
R¹ is selected from:

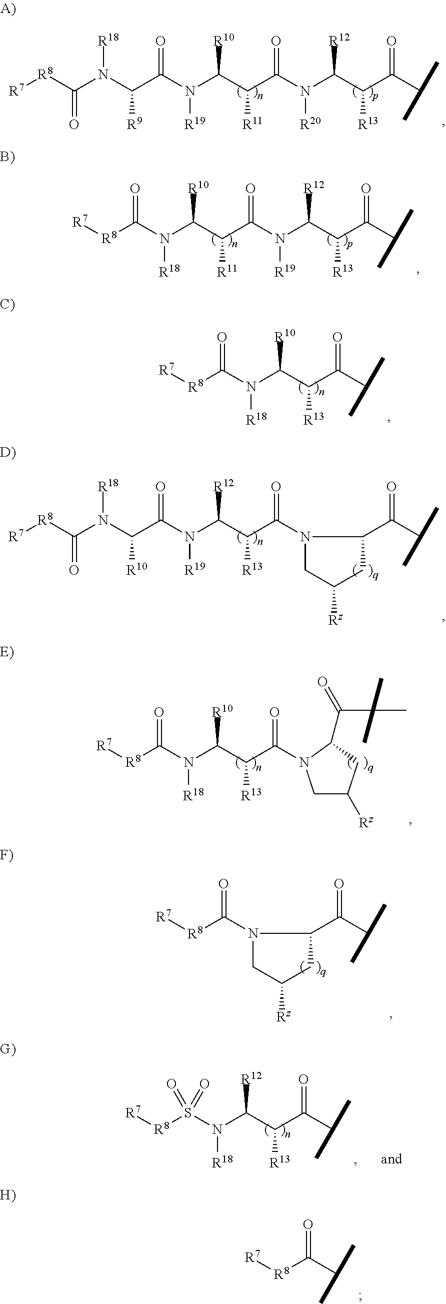

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

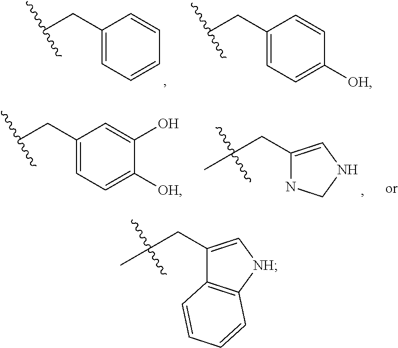

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

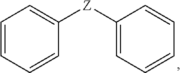

wherein Z is a bond, O, S, NH, CH₂, NHCH₂, or C≡C;
R⁸ is a bond, —O—, or —N(R¹⁷)—, optionally substituted C₁-C₆alkyl, optionally substituted C₁-C₆heteroalkyl, optionally substituted heterocycloalkyl, optionally substituted aryl, or optionally substituted heteroaryl;
R⁹ is —CH₂OH, —CH₂CH(CH₃)₂,

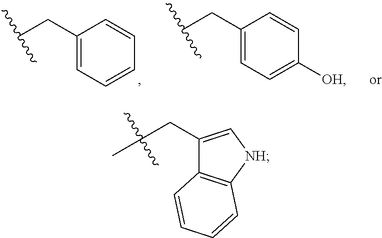 [SMILES: *Cc1ccccc1 ; *Cc1ccc(O)cc1 ; *Cc1c[nH]c2ccccc12]

R¹⁴ is C₁-C₆alkyl, C₁-C₆haloalkyl, or

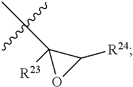

R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

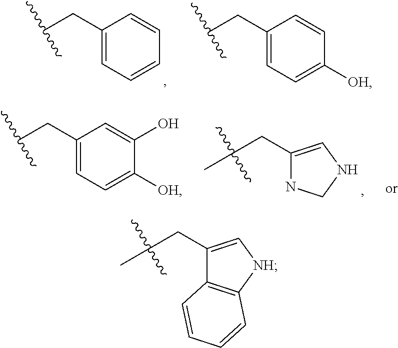

each R²⁵ is independently C₁-C₆alkyl;
R²⁶ is H, or C₁-C₄alkyl;
R²⁷ is H, or C₁-C₄alkyl;
n is 0 or 1;
p is 0 or 1; and
q is 0 or 1;
or a pharmaceutically acceptable salt, solvate, or prodrug thereof

In another embodiment is a compound of Formula (VIII) or Formula (VIII′) wherein R¹ is

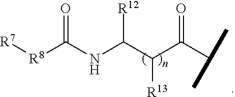

In another embodiment is a compound of Formula (VIII) or Formula (VIII′) wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

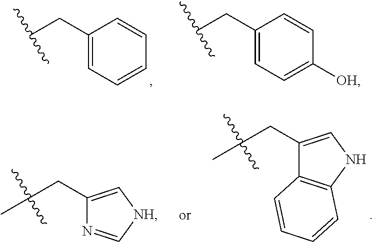 [SMILES: *Cc1ccccc1 ; *Cc1ccc(O)cc1 ; *Cc1c[nH]cn1 ; *Cc1c[nH]c2ccccc12]

In a further embodiment is a compound of Formula (VIII) or Formula (VIII′) wherein R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

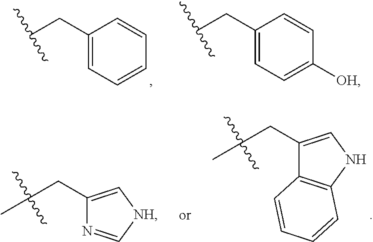 [SMILES: *Cc1ccccc1 ; *Cc1ccc(O)cc1 ; *Cc1c[nH]cn1 ; *Cc1c[nH]c2ccccc12]

In yet a further embodiment is a compound of Formula (VIII) or Formula (VIII′) wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

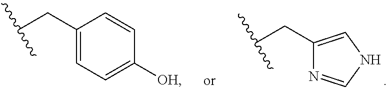 [SMILES: *Cc1ccc(O)cc1 ; *Cc1c[nH]cn1]

In a further embodiment is a compound of Formula (VIII) or Formula (VIII′) wherein R⁸ is a bond. In another embodiment is a compound of Formula (VIII) or Formula (VIII′) wherein R¹⁴ is

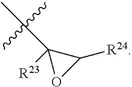

In another embodiment is a compound of Formula (VIII) or Formula (VIII′) wherein R²³ and R²⁴ are each H. In another embodiment is a compound of Formula (VIII) wherein R²³ is H and R²⁴ is CH₃. In another embodiment is a compound of Formula (VIII) or Formula (VIII′) wherein R²³ is CH₃ and R²⁴ is H. In another embodiment is a compound of Formula (VIII) or Formula (VIII′) wherein R¹⁴ is C₁-C₆haloalkyl. In another embodiment is a compound of Formula (VIII) or Formula (VIII′) wherein R¹⁴ is CF₃. In a further embodiment of the aforementioned embodiments is a compound of Formula (VIII) or Formula (VIII′) wherein n is 0. In yet a further embodiment, n is 1.

In a further embodiment is a compound of Formula (VIII) or Formula (VIII′) having the structure of Formula (VIIIa):

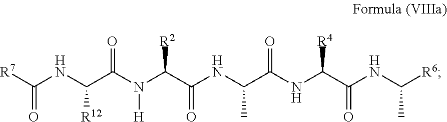

wherein R², R⁴, and R¹² are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂; and R⁶ and R⁷ are defined as above.

In another embodiment is a compound of Formula (VIIIa) wherein R⁴ is —(CH₂)₄NH₂, R² is —CH(OH)(CH₃), and R¹² is —(CH₂)₂NH₂. In another embodiment is a compound of Formula (VIIIa) wherein R⁴ is —(CH₂)₄NH₂, R² is —CH(OH)(CH₃), and R¹² is —CH₂NH₂. In another embodiment is a compound of Formula (VIIIa) wherein R⁴ is —CH₂C(O)NH₂, R² is —CH(OH)(CH₃), and R¹² is —(CH₂)₄NH₂. In another embodiment is a compound of Formula (VIIIa) wherein R⁴ is —(CH₂)₄NH₂, R² is —(CH₂)₄NH₂, and R¹² is —CH₂NH₂. In another embodiment is a compound of Formula (VIIIa) wherein R⁴ is —CH₂C(O)NH₂, R² is —(CH₂)₄NH₂, and R¹² is —CH₂NH₂. In another embodiment is a compound of Formula (VIIIa) wherein R⁴ is —CH₂CH(CH₃)₂, R² is —(CH₂)₂NH₂, and R¹² is —(CH₂)₂NH₂.

In a further embodiment is a compound of Formula (VIII′) having the structure of Formula (VIIIaa):

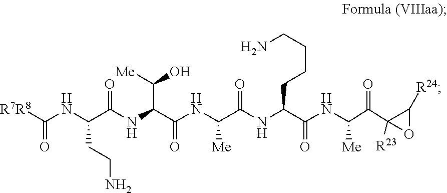

wherein R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

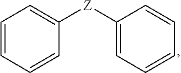

wherein Z is a bond, O, S, NH, CH₂, NHCH₂, or C≡C;
R⁸ is a bond;
R²³ is H, C₁-C₄alkyl, or C₁-C₄alkoxy; and
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

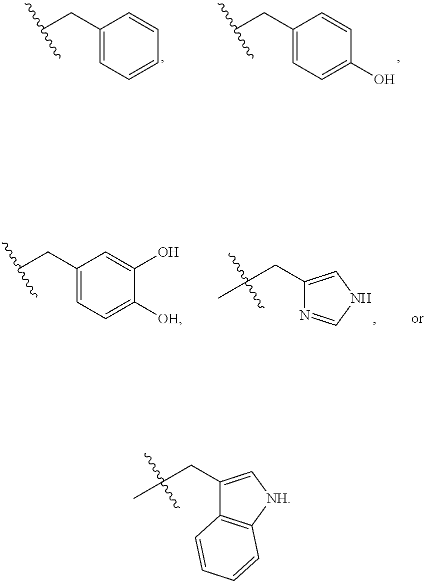 [SMILES: *Cc1ccccc1 ; *Cc1ccc(O)cc1 ; *Cc1ccc(O)c(O)c1 ; *Cc1c[nH]cn1 ; *Cc1c[nH]c2ccccc12]

In another embodiment is a compound of Formula (VIIIaa) wherein R⁷ is a linear or branched alkyl chain of 1-22 carbon atoms, optionally comprising within the alkyl chain or at an alkyl chain terminus an optionally substituted

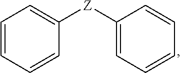

wherein Z is a bond; R²³ is H or C₁-C₄alkyl; and R²⁴ is H or C₁-C₄alkyl.

In another embodiment is a compound of Formula (VIII) or Formula (VIII′) wherein R¹ is

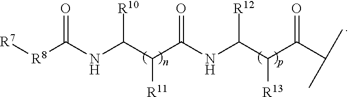

In a further embodiment, R⁸ is a bond. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

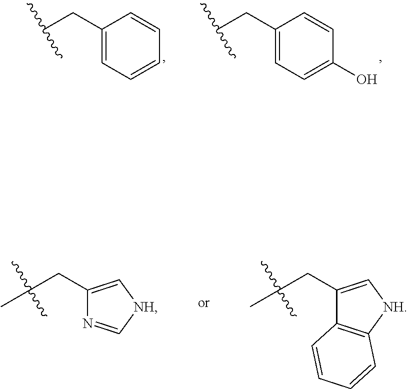 [SMILES: *Cc1ccccc1 ; *Cc1ccc(O)cc1 ; *Cc1c[nH]cn1 ; *Cc1c[nH]c2ccccc12]

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

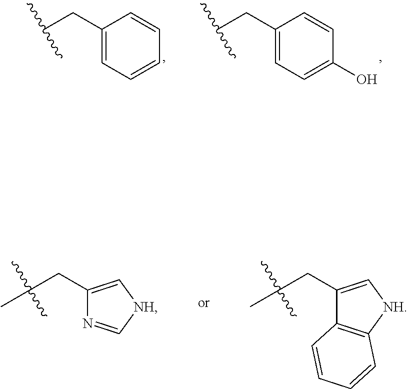 [SMILES: *Cc1ccccc1 ; *Cc1ccc(O)cc1 ; *Cc1c[nH]cn1 ; *Cc1c[nH]c2ccccc12]

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂, or

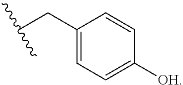 [SMILES: *Cc1ccc(O)cc1]

In a further embodiment of the aforementioned embodiments is a compound of Formula (VIII) or Formula (VIII′) wherein n is 0 and p is 0. In another embodiment, n is 0 and p is 1. In yet a further embodiment, n is 1 and p is 0.

In a further embodiment is a compound of Formula (VIII′) having the structure of Formula (VIIIb):

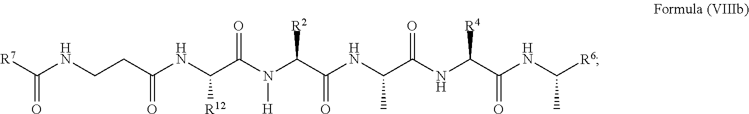

wherein R², R⁴, and R¹², are each independently —CH₂CH(CH₃)₂, —(CH₂)₃NH₂, or —(CH₂)₄NH₂.

In another embodiment is a compound of Formula (VIIIb) wherein R², R⁴, and R¹² are each —(CH₂)₄NH₂. In another embodiment is a compound of Formula (VIIIb) wherein R², R⁴, and R¹² are each —(CH₂)₃NH₂. In another embodiment is a compound of Formula (VIIIb) wherein R⁴ is —CH₂CH(CH₃)₂, R² is —(CH₂)₃NH₂, and R¹² is —(CH₂)₄NH₂. In another embodiment is a compound of Formula (VIIIb) wherein R⁴ is —CH₂CH(CH₃)₂, R² is —(CH₂)₄NH₂, and R¹² is —(CH₂)₄NH₂.

In a further embodiment is a compound of Formula (VIII′) having the structure of Formula (VIIIbb):

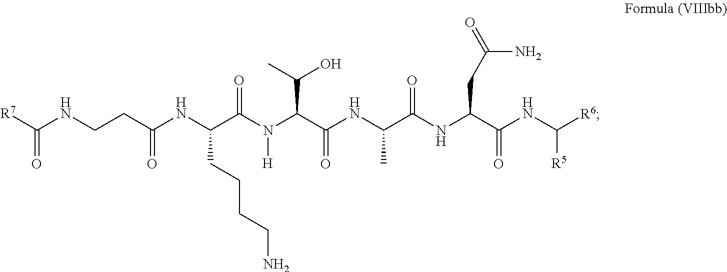

wherein R⁵ is —H, or —CH₃.

In a further embodiment is a compound of Formula (VIII′) having the structure of Formula (VIIIbbb):

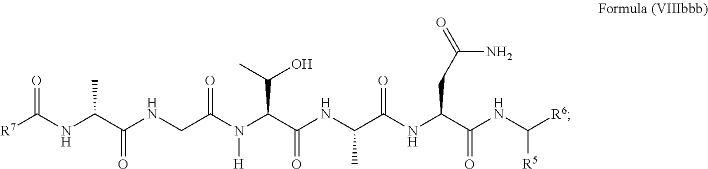

wherein R⁵ is —H, or —CH₃.

In another embodiment is a compound of Formula (VIII) or Formula (VIII′) wherein R¹ is

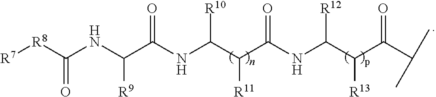

In a further embodiment, R⁸ is a bond. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, (CH₂)₄NH₂,

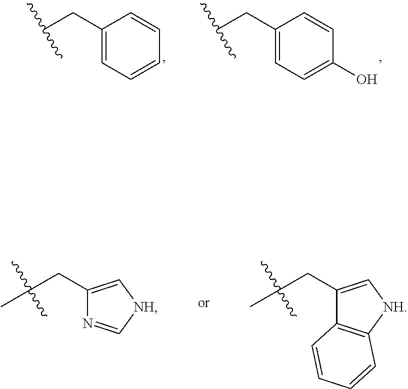 [SMILES: *Cc1ccccc1 ; *Cc1ccc(O)cc1 ; *Cc1c[nH]cn1 ; *Cc1c[nH]c2ccccc12]

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

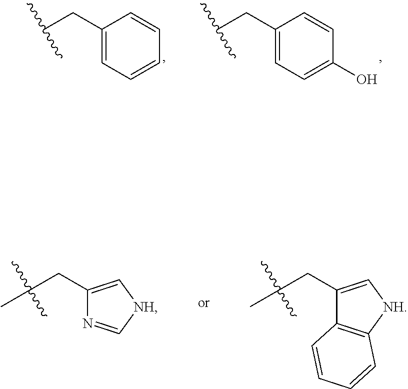 [SMILES: *Cc1ccccc1 ; *Cc1ccc(O)cc1 ; *Cc1c[nH]cn1 ; *Cc1c[nH]c2ccccc12]

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

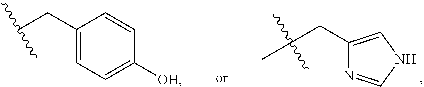 [SMILES: *Cc1ccc(O)cc1 ; *Cc1c[nH]cn1]

In a further embodiment of the aforementioned embodiments is a compound of Formula (VIII) or Formula (VIII′) wherein n is 0 and p is 0. In another embodiment, n is 0 and p is 1. In yet a further embodiment, n is 1 and p is 0.

In a further embodiment is a compound of Formula (VIII′) having the structure of Formula (VIIIc):

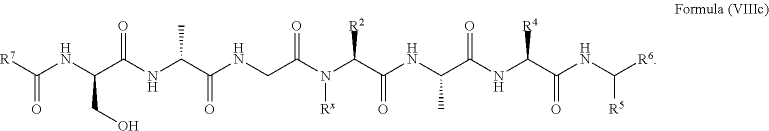

In another embodiment is a compound of Formula (VIIIc) wherein R² is —CH(OH)(CH₃), —CH₂CH₂C(O)OH, or —(CH₂)₄NH₂. In some embodiments, R² is —CH(OH)(CH₃). In some embodiments, R² is —CH₂CH₂C(O)OH. In some embodiments, R² is —(CH₂)₄NH₂. In a further embodiment is a compound of Formula (VIIIc) wherein R⁴ is CH₂CH(CH₃)₂ or —CH₂C(O)NH₂. In some embodiments, R⁴ is CH₂CH(CH₃)₂. In some embodiments, R⁴ is —CH₂C(O)NH₂. In yet a further embodiment is a compound of Formula (VIIIc) wherein R⁵ is H or —CH₃. In some embodiments, R⁴ is H. In some embodiments, R⁴ is —CH₃.

In another embodiment is a compound of Formula (VIII) or Formula (VIII′) wherein R¹ is

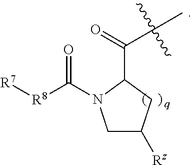

In a further embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

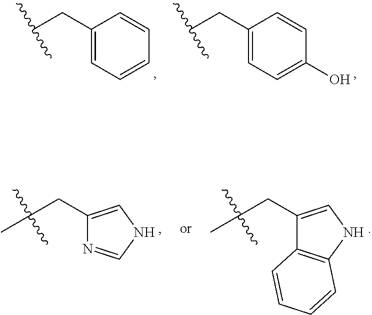

In a further embodiment, q is 1 and R⁸ is a bond.

In a further embodiment is a compound of Formula (VIII′) having the structure of Formula (VIIId):

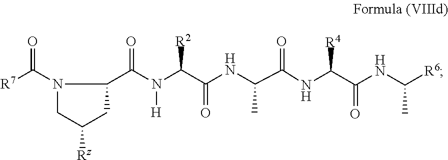

wherein R^{z} is NH₂; and R² and R⁴ are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (VIIId) wherein R² is —CH(OH)(CH₃), and R⁴ is —CH₂C(O)NH₂. In another embodiment is a compound of Formula (VIIId) wherein R² is —CH(OH)(CH₃), and R⁴ is —(CH₂)₂NH₂. In another embodiment is a compound of Formula (VIIId) wherein R² is —CH(OH)(CH₃), and R⁴ is —(CH₂)₃NH₂. In another embodiment is a compound of Formula (VIIId) wherein R² is —CH(OH)(CH₃), and R⁴ is —(CH₂)₄NH₂. In another embodiment is a compound of Formula (VIIId) wherein R² is —(CH₂)₄NH₂ and R⁴ is —CH₂CH(CH₃)₂. In another embodiment is a compound of Formula (VIIId) wherein R² is —(CH₂)₄NH₂ and R⁴ is —CH₂C(O)NH₂. In another embodiment is a compound of Formula (VIIId) wherein R² is —(CH₂)₄NH₂ and R⁴ is —(CH₂)₄NH₂.

In another embodiment is a compound of Formula (VIII) or Formula (VIII′) wherein R¹ is

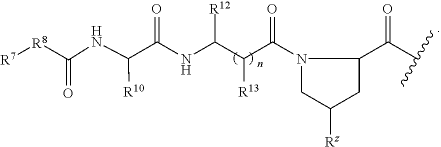

In a further embodiment, R⁸ is a bond. In another embodiment, R², R⁴, R¹⁰, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

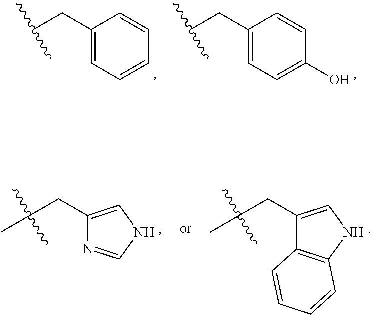

In a further embodiment, R², R⁴, R¹⁰, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

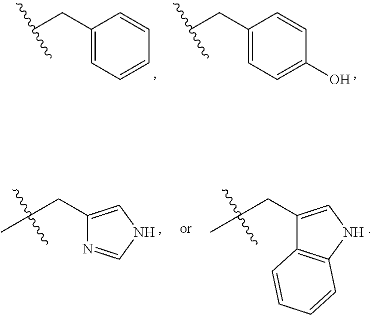

In yet a further embodiment, R², R⁴, R¹⁰, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

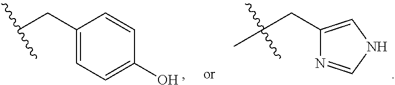 [SMILES: *Cc1ccc(O)cc1 ; *Cc1c[nH]cn1]

In a further embodiment of the aforementioned embodiments is a compound of Formula (VIII) or Formula (VIII′) wherein n is 0. In yet a further embodiment, n is 1.

In a further embodiment is a compound of Formula (VIII′) having the structure of Formula (VIIIdd):

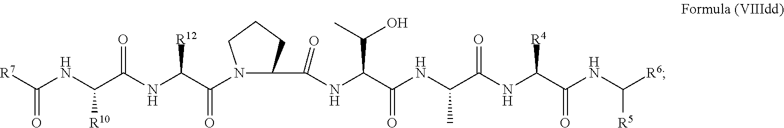

wherein R⁵ is —H, or —CH₃.

In another embodiment is a compound of Formula (VIIIdd) wherein R¹⁰ is —CH₂OH, and R¹² is —CH₃. In another embodiment is a compound of Formula (VIIIdd) wherein R¹⁰ is —CH₂CH(CH₃)₂, and R¹² is —CH(OH)(CH₃). In another embodiment of the aforementioned compounds of Formula (VIIId) is a compound wherein R⁴ is —CH₂C(O)NH₂. In yet another embodiment of the aforementioned compounds of Formula (VIIIdd) is a compound wherein R⁴ is

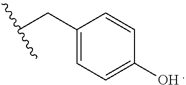 [SMILES: *Cc1ccc(O)cc1]

In another embodiment is a compound of Formula (VIII) or Formula (VIII′) wherein R¹ is

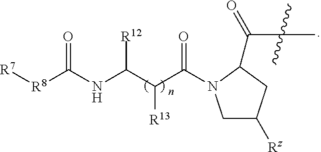

In a further embodiment, R⁸ is a bond. In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

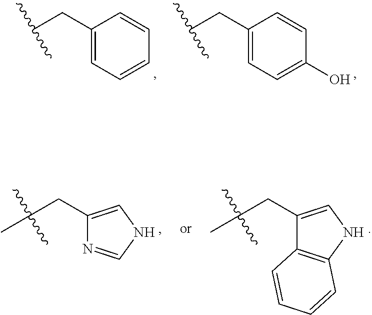

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

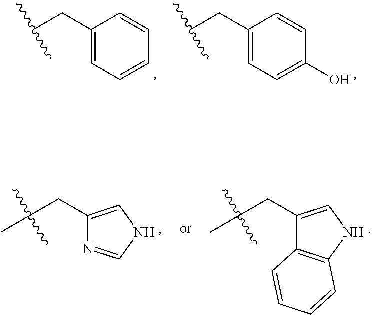

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂NH₂, —CH₂CH₂C(O)NH₂, —(CH₂)₂NH₂, —(CH₂)₃NH₂, —(CH₂)₄NH₂.

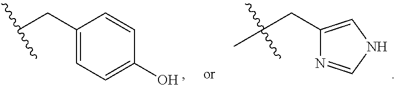 [SMILES: *Cc1ccc(O)cc1 ; *Cc1c[nH]cn1]

In a further embodiment of the aforementioned embodiments is a compound of Formula (VIII) or Formula (VIII′) wherein n is 0. In yet a further embodiment, n is 1.

In another embodiment is a compound of Formula (VIII) or Formula (VIII′) wherein R¹ is

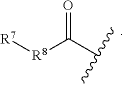

In a further embodiment, R⁸ is a bond. In another embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

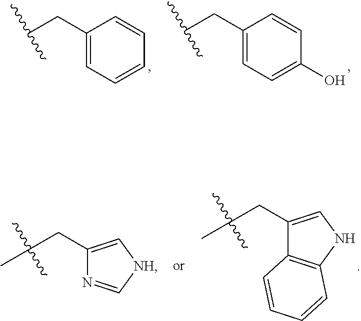

In a further embodiment, R² and R⁴ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

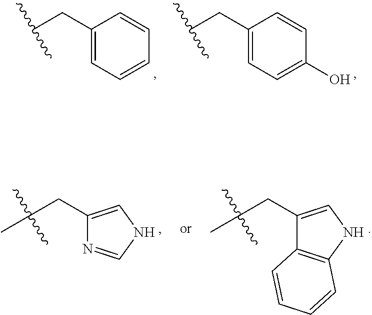

In yet a further embodiment, R² and R⁴ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

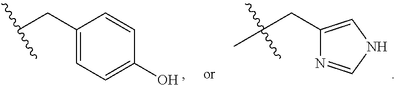 [SMILES: *Cc1ccc(O)cc1 ; *Cc1c[nH]cn1]

In another embodiment is a compound of Formula (VIII) or Formula (VIII′) wherein R^{x} and R² together with the nitrogen atom form an optionally substituted nitrogen containing ring. In a further embodiment is a compound of Formula (VIII′) having the structure of Formula (VIIIe):

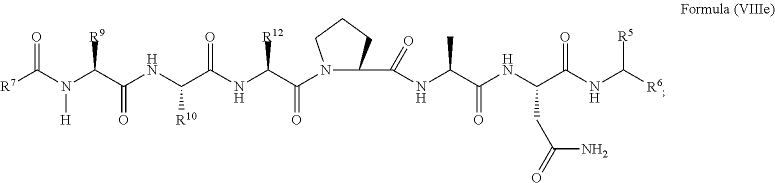

wherein R⁵ is —H, or —CH₃.

In another embodiment is a compound of Formula (VIIIe) wherein R¹⁰ and R¹² are each independently —H, —CH₃, —CH₂CH(CH₃)₂, —CH₂OH, or —CH(OH)(CH₃).

In another aspect described herein are compounds of Formula (IX):

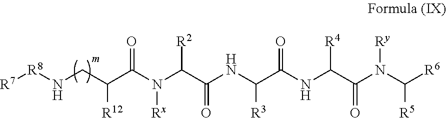

wherein:
R², R⁴, and R¹² are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —NR²¹R²², —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

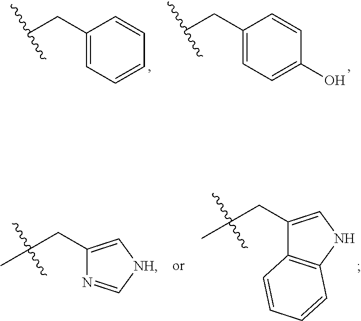

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

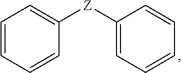

wherein Z is a bond, O, S, NH, CH₂, NHCH₂, or C≡C;
R⁸ is a bond, C(O), optionally substituted C₁-C₆alkyl, optionally substituted C₁-C₆heteroalkyl, optionally substituted aryl, optionally substituted heteroaryl, or optionally substituted heterocycloalkyl;
R¹⁴ is C₁-C₆alkyl, C₁-C₆haloalkyl, or

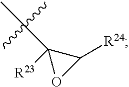

each R²¹ is independently H, or C₁-C₄alkyl;
each R²² is independently H, C₁-C₄alkyl, —C(═O)R²⁸, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

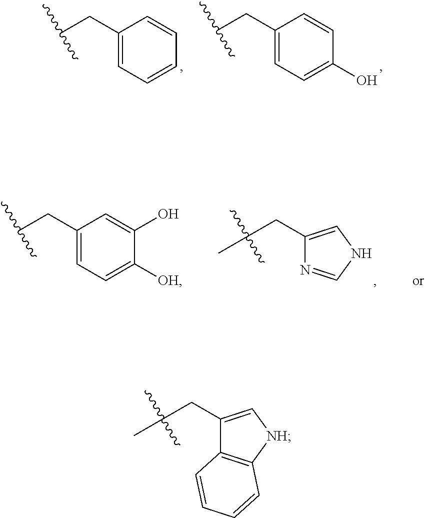

each R²⁵ is independently C₁-C₆alkyl;
R²⁶ is H, or C₁-C₄alkyl;
R²⁷ is H, or C₁-C₄alkyl;
R²⁸ is H, or optionally substituted C₁-C₆alkyl; and
m is 0-4;
or a pharmaceutically acceptable salt, solvate, or prodrug thereof

In another embodiment is a compound of Formula (IX) having the structure of Formula (IX′):

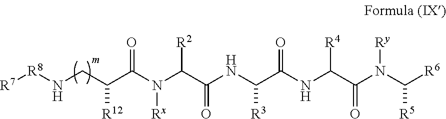

wherein:
R², R⁴, and R¹² are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —NR²¹R²², —CH₂NR²¹R²², —(CH₂)₂NR²¹R²², —(CH₂)₃NR²¹R²², —CH₂ optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

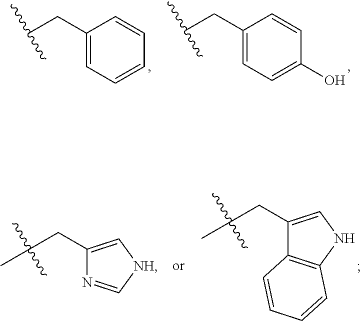

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

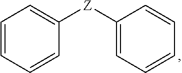

wherein Z is a bond, O, S, NH, CH₂, NHCH₂, or C≡C;
R⁸ is a bond, C(O), optionally substituted C₁-C₆alkyl, optionally substituted C₁-C₆heteroalkyl, optionally substituted aryl, optionally substituted heteroaryl, or optionally substituted heterocycloalkyl;
R¹⁴ is C₁-C₆alkyl, C₁-C₆haloalkyl, or

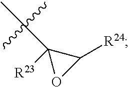

each R²¹ is independently H, or C₁-C₄alkyl;
each R²² is independently H, C₁-C₄alkyl, —C(═O)R²⁸, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

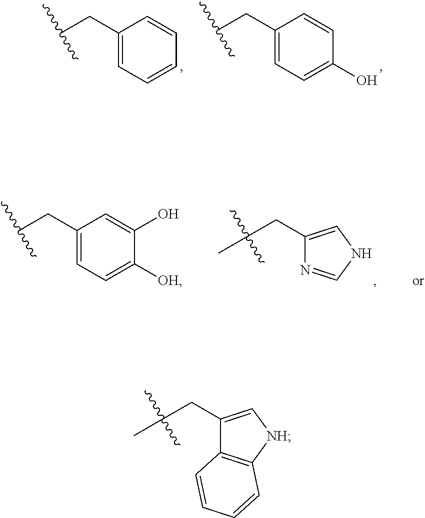

each R²⁵ is independently C₁-C₆alkyl;
R²⁶ is H, or C₁-C₄alkyl;
R²⁷ is H, or C₁-C₄alkyl;
R²⁸ is H, or optionally substituted C₁-C₆alkyl; and
m is 0-4;
or a pharmaceutically acceptable salt, solvate, or prodrug thereof

In a further embodiment is a compound of Formula (IX) or Formula (IX′) wherein R⁸ is a bond. In another embodiment of Formula (IX) or Formula (IX′), R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

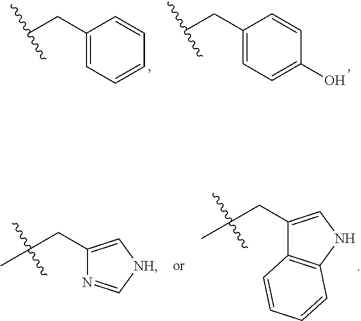

In a further embodiment, R² and R⁴ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

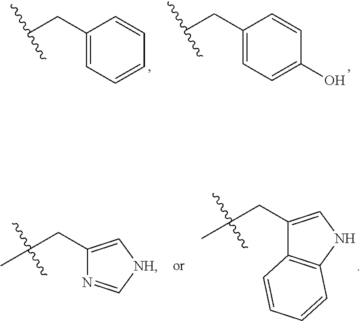

In yet a further embodiment, R² and R⁴ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

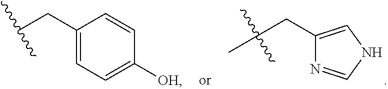 [SMILES: *Cc1ccc(O)cc1 ; *Cc1c[nH]cn1]

In another aspect described herein are compounds of Formula (X):

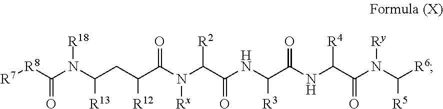

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

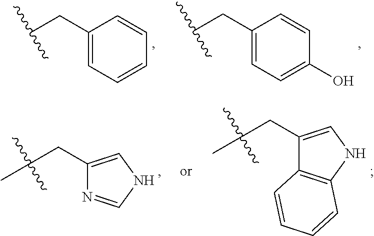

R¹² and R¹³ are each independently —H, —NR²¹R²², —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, or optionally substituted C₁-C₈heteroalkyl; or R¹² and R¹³ together with the carbon atoms to which they are attached form a heterocycloalkyl ring;
R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

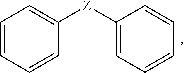

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹⁴ is C₁-C₆alkyl, C₁-C₆haloalkyl, or

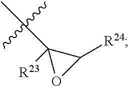

R¹⁸ is H, or methyl; or R¹⁸ and R¹² together with the atoms to which they are attached form a heterocycloalkyl ring;
each R²¹ is independently H, or C₁-C₄alkyl;
each R²² is independently H, C₁-C₄alkyl, —C(═O)R²⁸, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —CH₂, —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

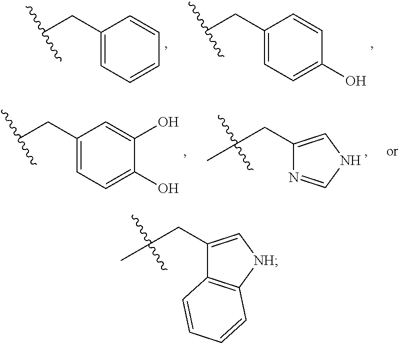

each R²⁵ is independently C₁-C₆alkyl;
R²⁶ is H, or C₁-C₄alkyl;
R²⁷ is H, or C₁-C₄alkyl;
R²⁸ is H, or optionally substituted C₁-C₆alkyl; and
m is 0-4;
or a pharmaceutically acceptable salt, solvate, or prodrug thereof

In another embodiment is a compound of Formula (X) having the structure of Formula (X′):

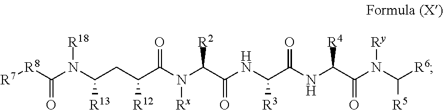

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

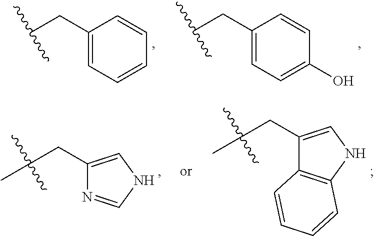

R¹² and R¹³ are each independently —H, —NR²¹R²², —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, or optionally substituted C₁-C₈heteroalkyl; or R¹² and R¹³ together with the carbon atoms to which they are attached form a heterocycloalkyl ring;
R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

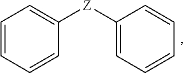

wherein Z is a bond, O, S, NH, CH₂, NHCH₂, or C≡C;
R⁸ is a bond, optionally substituted C₁-C₆alkyl, optionally substituted C₁-C₆heteroalkyl, optionally substituted aryl, optionally substituted heteroaryl, or optionally substituted heterocycloalkyl;
R¹⁴ is C₁-C₆alkyl, C₁-C₆haloalkyl, or

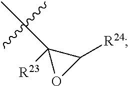

R¹⁸ is H, or methyl; or R¹⁸ and R¹² together with the atoms to which they are attached form a heterocycloalkyl ring;
each R²¹ is independently H, or C₁-C₄alkyl;
each R²² is independently H, C₁-C₄alkyl, —C(═O)R²⁸, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

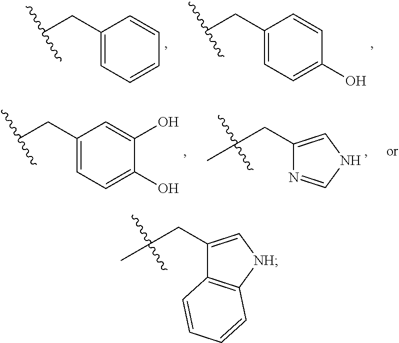

each R²⁵ is independently C₁-C₆alkyl;
R²⁶ is H, or C₁-C₄alkyl;
R²⁷ is H, or C₁-C₄alkyl;
R²⁸ is H, or optionally substituted C₁-C₆alkyl; and
m is 0-4;
   or a pharmaceutically acceptable salt, solvate, or prodrug thereof

In some embodiments is a compound of Formula (X) or Formula (X′) wherein R⁸ is a bond. In a further embodiment is a compound of Formula (X) or Formula (X′) wherein R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²². In yet a further embodiment is a compound of Formula (X) or Formula (X′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment is a compound of Formula (X) or Formula (X′) wherein R¹² and R¹³ together with the carbon atoms to which they are attached form a heterocycloalkyl ring. In a further embodiment is a compound of Formula (X) or Formula (X′) wherein R¹² and R¹³ together with the carbon atoms to which they are attached form a pyrrolidine ring. In yet a further embodiment is a compound of Formula (X) or Formula (X′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment is a compound of Formula (X) or Formula (X′) wherein R¹⁸ and R¹² together with the atoms to which they are attached form a heterocycloalkyl ring. In a further embodiment is a compound of Formula (X) or Formula (X′) wherein R¹⁸ and R¹² together with the atoms to which they are attached form a piperidine ring In yet a further embodiment is a compound of Formula (X) or Formula (X′) wherein R¹³ is H and R² and R⁴ are each independently —CH(CH₃)₂, —CH(CH₃)(CH₂CH₃), —CH₂CH(CH₃)₂, —CH₂OH, —CH(OH)(CH₃), —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment of the aforementioned embodiments of Formula (VIII), (VIII′), (IX), (IX′), (X), or (X′) is a compound wherein R⁸ is a bond and R⁷ is a linear or branched alkyl chain of about 1-22 carbon atoms. In another embodiment of the aforementioned embodiments of Formula (VIII), (VIII′), (IX), (IX′), (X), or (X′) is a compound wherein R⁸ is a bond and R⁷ is

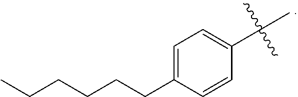 [SMILES: *c1ccc(CCCCCC)cc1]

In another embodiment of the aforementioned embodiments of Formula (VIII), (VIII′), (IX), (IX′), (X), or (X′) is a compound wherein R⁸ is a bond and R⁷ is

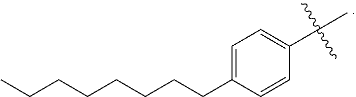 [SMILES: *c1ccc(CCCCCCCC)cc1]

In another embodiment of the aforementioned embodiments of Formula (VIII), (VIII′), (IX), (IX′), (X), or (X′) is a compound wherein R⁸ is a bond and R⁷ is

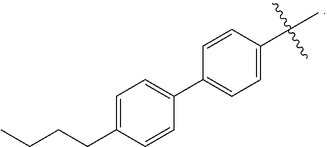 [SMILES: *c1ccc(-c2ccc(CCCC)cc2)cc1]

In another embodiment of the aforementioned embodiments of Formula (VIII), (VIII′), (IX), (IX′), (X), or (X′) is a compound wherein R⁸ is a bond and R⁷ is

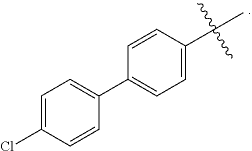 [SMILES: *c1ccc(-c2ccc(Cl)cc2)cc1]

In another embodiment of the aforementioned embodiments of Formula (VIII), (VIII′), (IX), (IX′), (X), or (X′) is a compound wherein R¹⁴ is

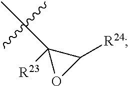

R²³ is H or C₁-C₄alkyl; and R²⁴ is H or C₁-C₄alkyl.

In another aspect is a compound selected from:

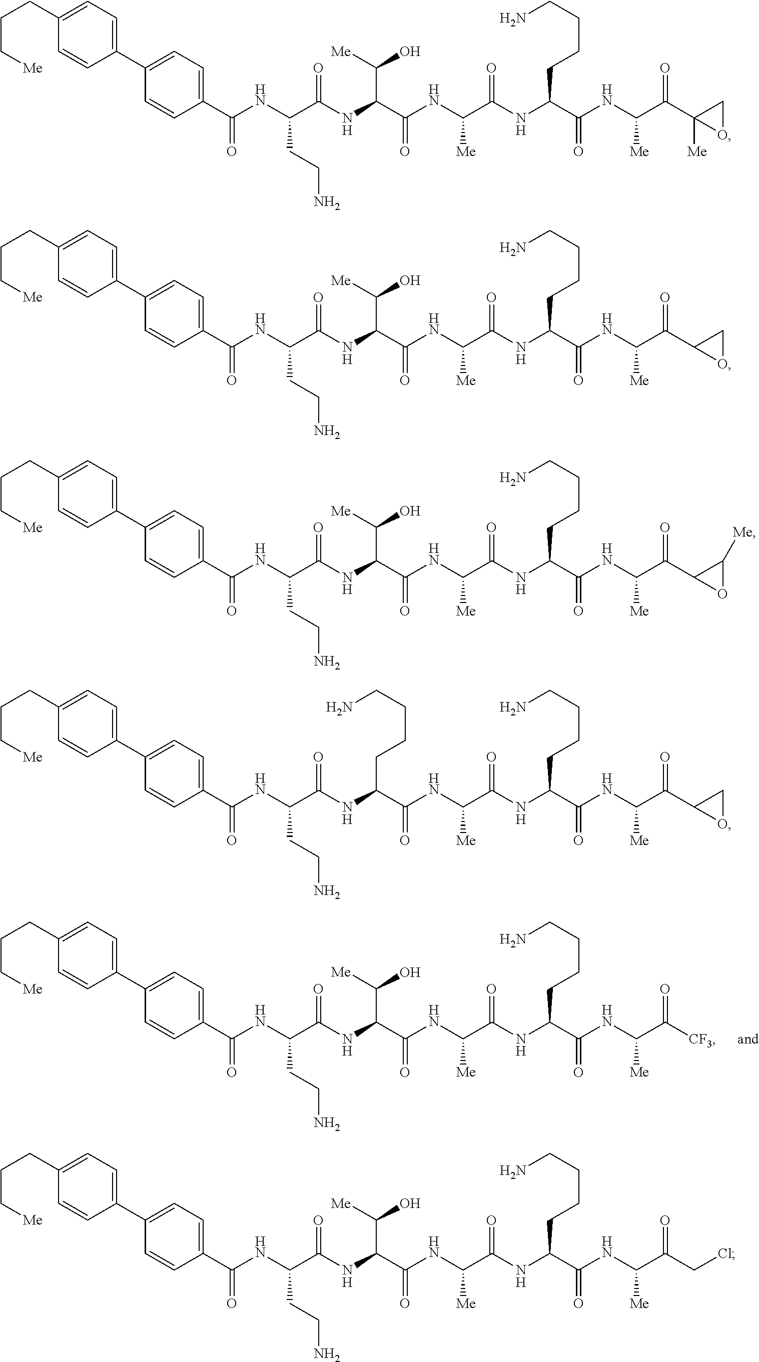

or a pharmaceutically acceptable salt, solvate, or prodrug thereof.

In another aspect is a compound selected from:

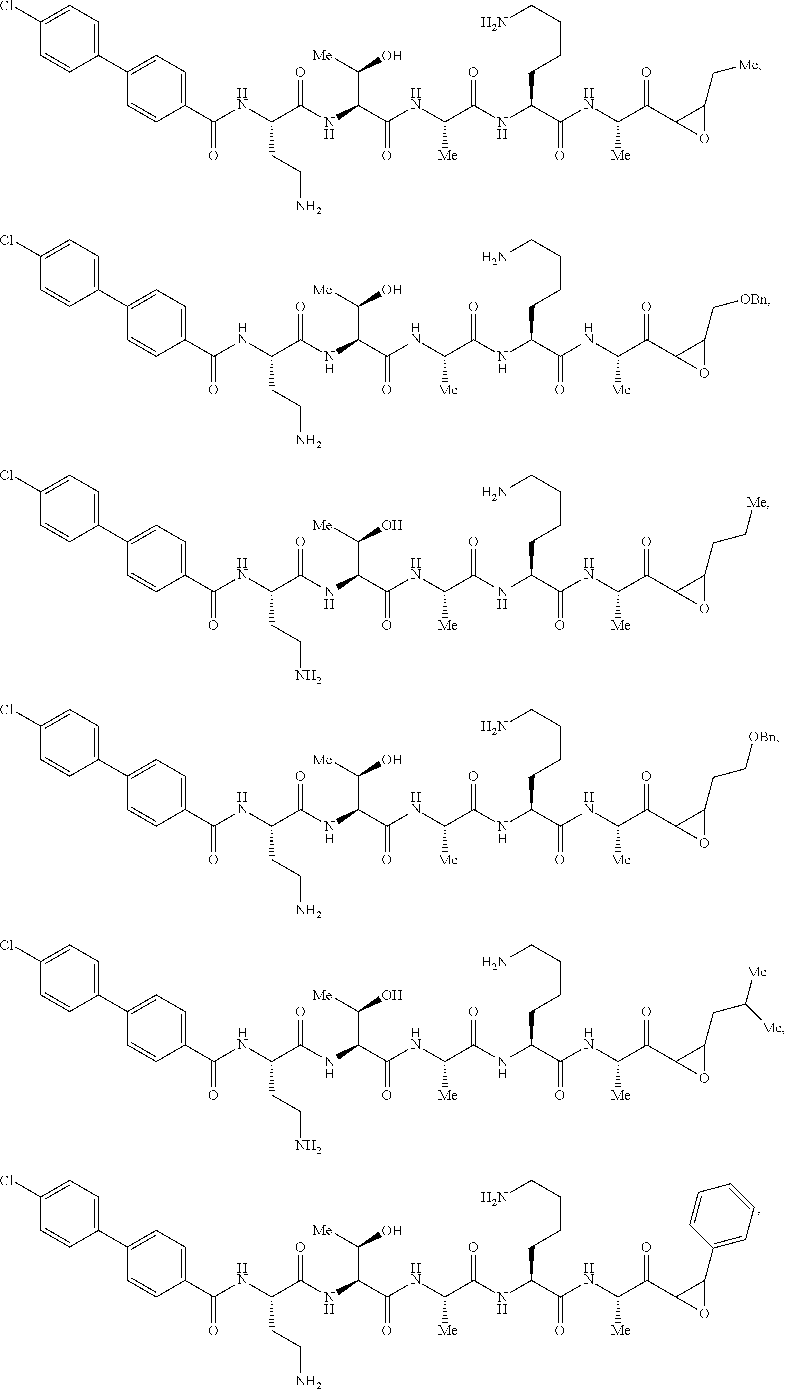

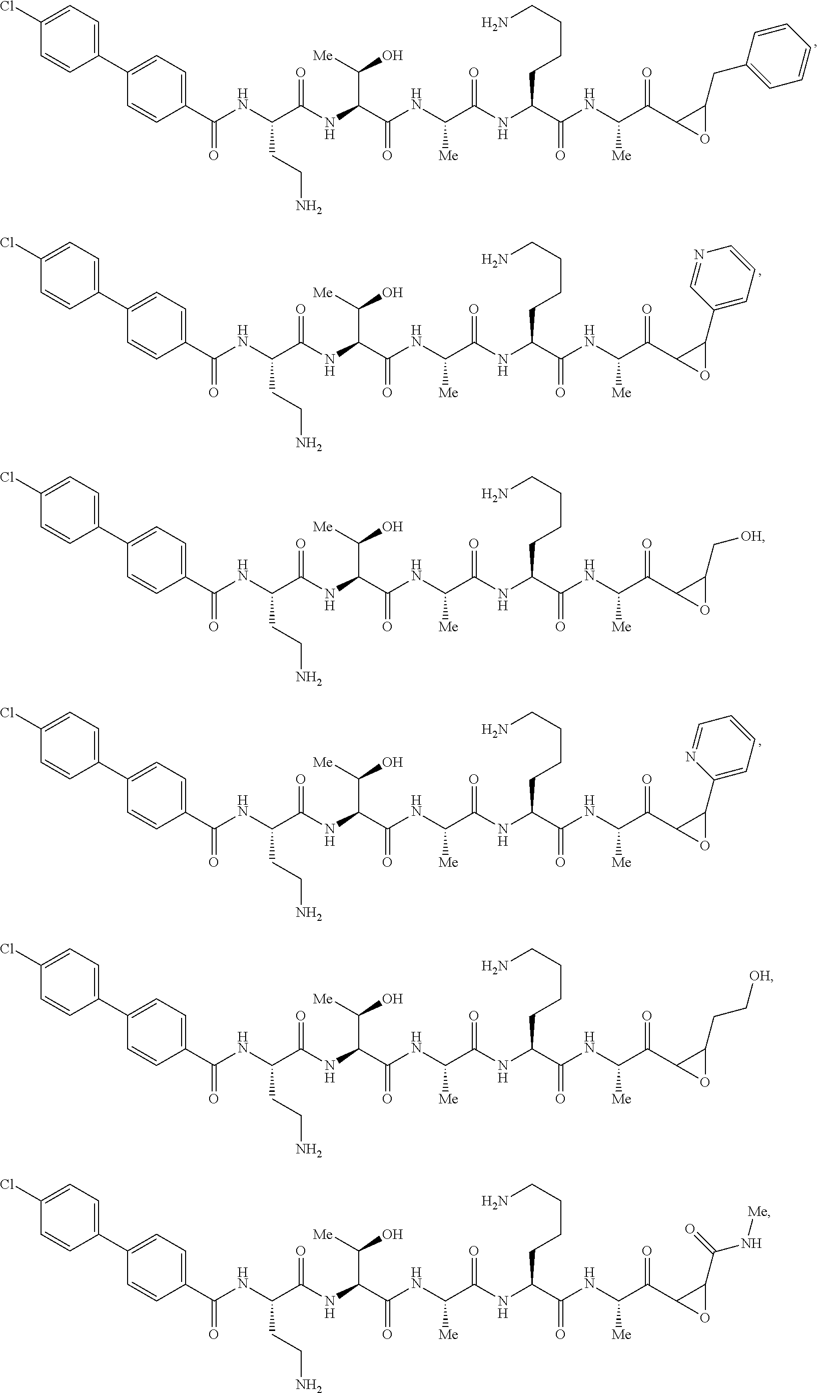

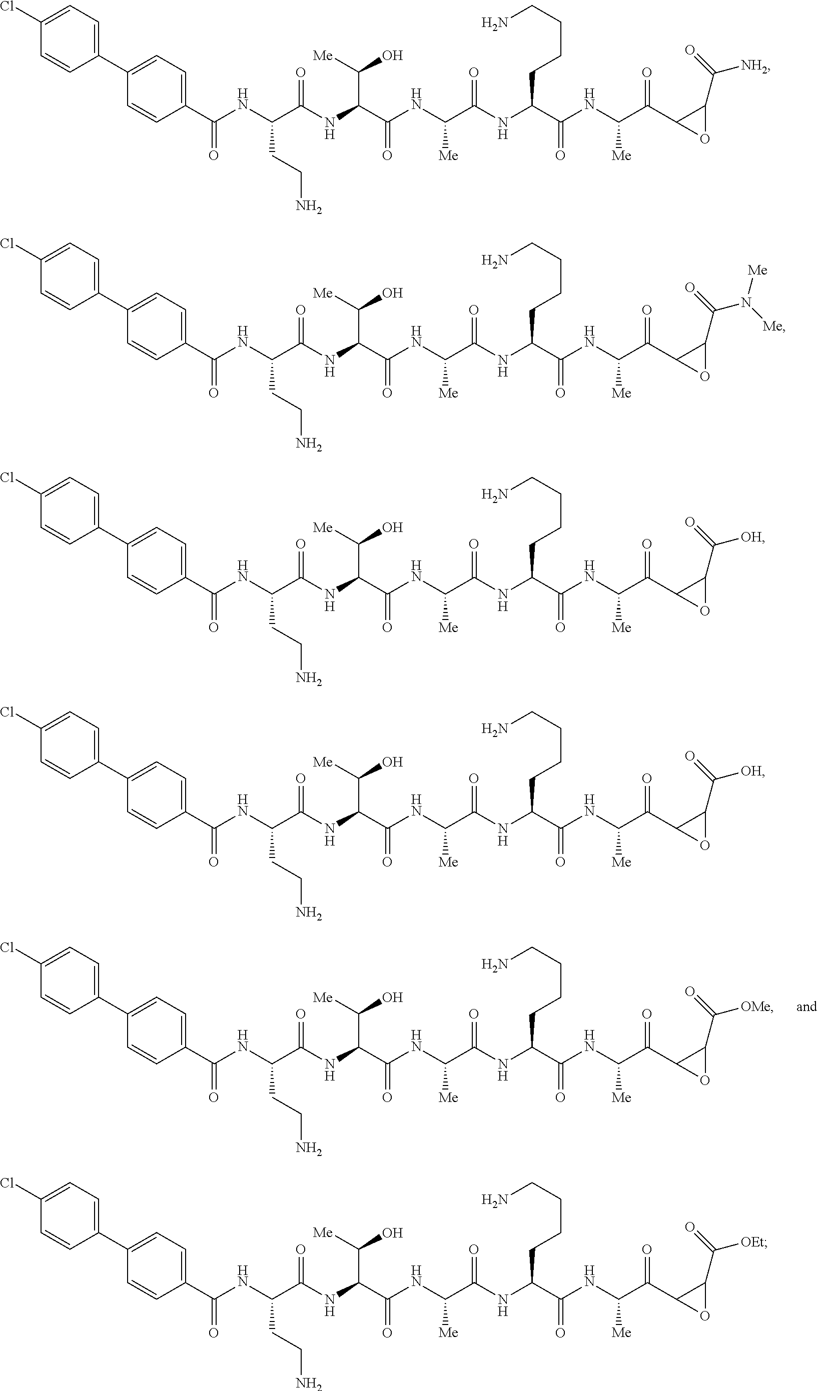

or a pharmaceutically acceptable salt, solvate, or prodrug thereof.

In another aspect described herein are compounds of Formula (XI):

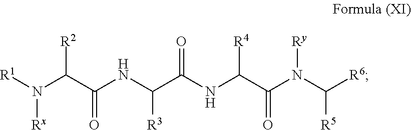

wherein:
R¹ is selected from:

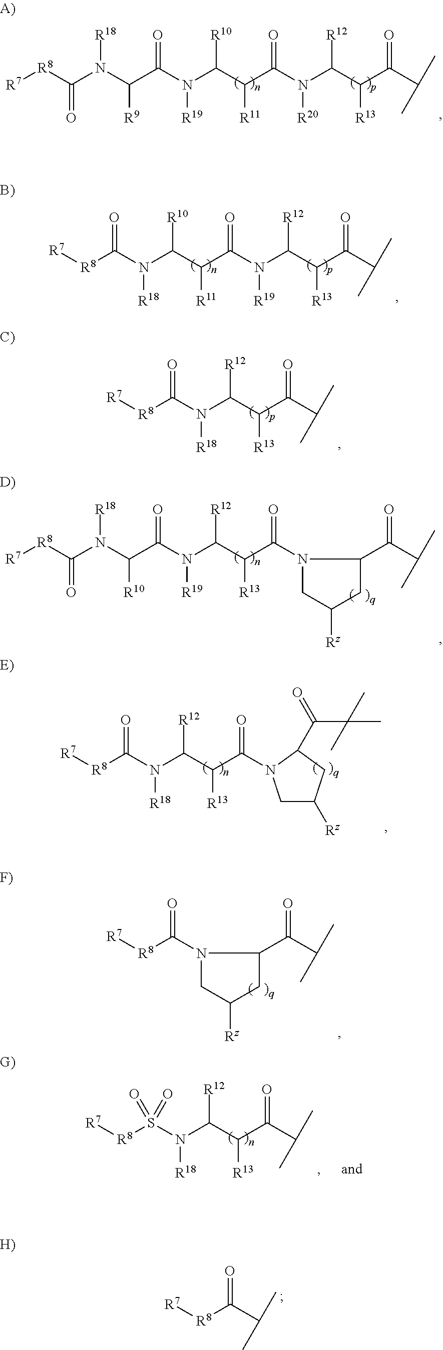

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

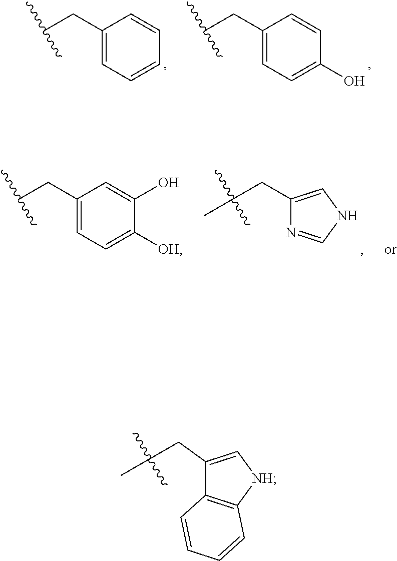

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

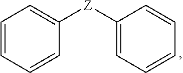

wherein Z is a bond, O, S, NH, CH₂, NHCH₂, or C≡C;
R⁸ is a bond, —O—, —N(R¹⁷)—, optionally substituted C₁-C₆alkyl, optionally substituted C₁-C₆heteroalkyl, optionally substituted heterocycloalkyl, optionally substituted aryl, or optionally substituted heteroaryl;
R⁹ is —CH₂OH, —CH₂CH(CH₃)₂,

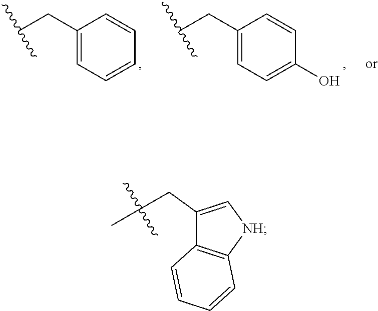

R¹⁴ is optionally substituted heteroaryl;
R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
each R²⁵ is independently C₁-C₆alkyl;
n is 0 or 1;
p is 0 or 1; and
q is 0 or 1;
or a pharmaceutically acceptable salt, solvate, or prodrug thereof

In one embodiment is a compound of Formula (XI) having the structure of Formula (XI′):

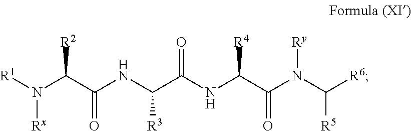

wherein:
R¹ is selected from:

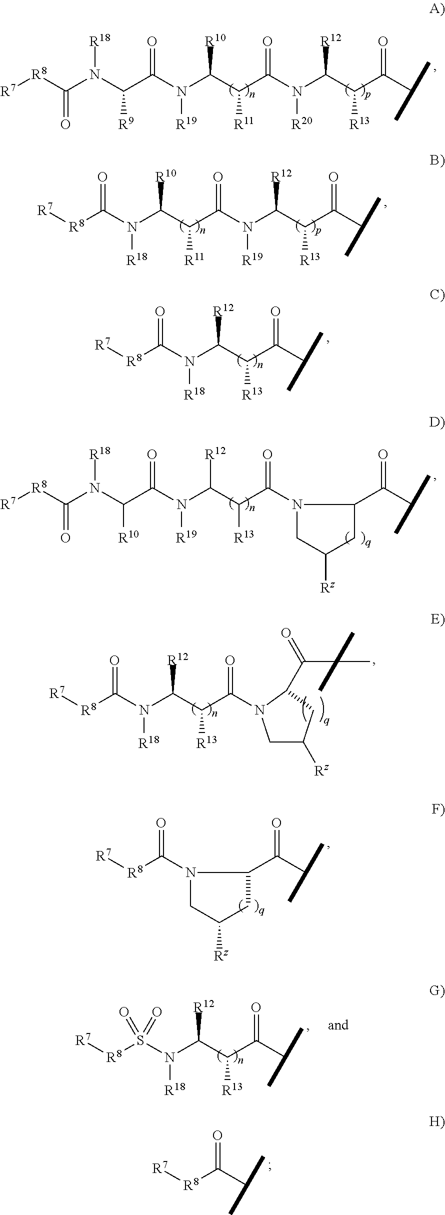

R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

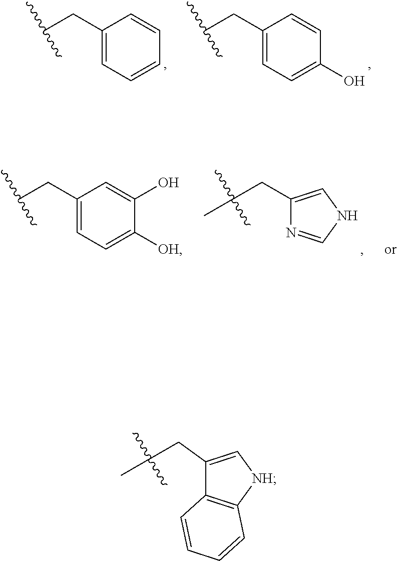

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

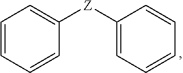

wherein Z is a bond, O, S, NH, CH₂, NHCH₂, or C≡C;
R⁸ is a bond, —O—, —N(R¹⁷)—, optionally substituted C₁-C₆alkyl, optionally substituted C₁-C₆heteroalkyl, optionally substituted heterocycloalkyl, optionally substituted aryl, or optionally substituted heteroaryl;
R⁹ is —CH₂OH, —CH₂CH(CH₃)₂,

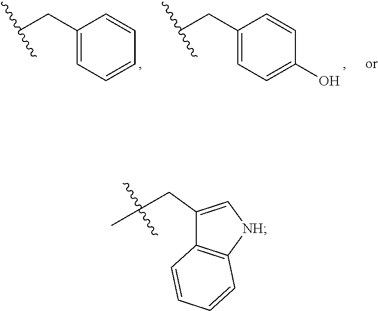

R¹⁴ is optionally substituted heteroaryl;
R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
each R²⁵ is independently C₁-C₆alkyl;
n is 0 or 1;
p is 0 or 1; and
q is 0 or 1;
or a pharmaceutically acceptable salt, solvate, or prodrug thereof

In one embodiment is a compound of Formula (XI) or Formula (XI′) wherein R¹ is

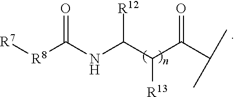

In a further embodiment, R⁸ is a bond. In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

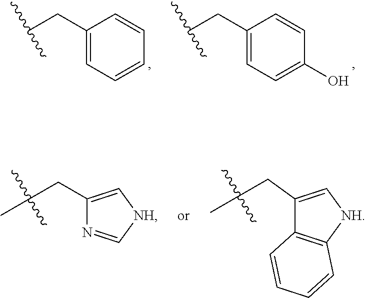

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

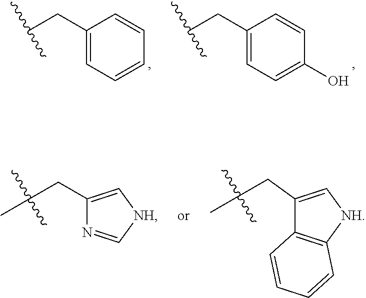

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

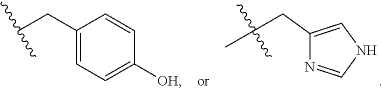 [SMILES: *Cc1ccc(O)cc1 ; *Cc1c[nH]cn1]

In a further embodiment is a compound of Formula (XI′) having the structure of Formula (XIa):

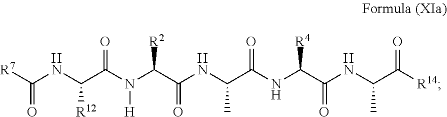

wherein R², R⁴, and R¹², are each independently —CH₂CH(CH₃)₂, —CH(OH)(CH₃), —CH₂C(O)NH₂, CH₂CH₂C(O)NH₂, —CH₂NH₂, —(CH₂)₂NH₂, —(CH₂)₃NH₂, or —(CH₂)₄NH₂.

In another embodiment is a compound of Formula (XIa) wherein R⁴ is —(CH₂)₄NH₂, R² is —CH(OH)(CH₃), and R¹² is —(CH₂)₂NH₂. In another embodiment is a compound of Formula (XIa) wherein R⁴ is —(CH₂)₄NH₂, R² is —CH(OH)(CH₃), and R¹² is —CH₂NH₂. In another embodiment is a compound of Formula (XIa) wherein R⁴ is —CH₂C(O)NH₂, R² is —CH(OH)(CH₃), and R¹² is —(CH₂)₄NH₂. In another embodiment is a compound of Formula (XIa) wherein R⁴ is —(CH₂)₄NH₂, R² is —(CH₂)₄NH₂, and R¹² is —CH₂NH₂. In another embodiment is a compound of Formula (XIa) wherein R⁴ is —CH₂C(O)NH₂, R² is —(CH₂)₄NH₂, and R¹² is —CH₂NH₂. In another embodiment is a compound of Formula (XIa) wherein R⁴ is —CH₂CH(CH₃)₂, R² is —(CH₂)₂NH₂, and R¹² is —(CH₂)₂NH₂. In another embodiment of the aforementioned embodiments of Formula (XIa) is a compound wherein R¹⁴ is an optionally substituted heteroaryl selected from furan, thiophene, pyrrole, pyridine, oxazole, thiazole, imidazole, isoxazole, isothiazole, pyrazole, pyridazine, pyrimidine, pyrazine, oxadiazole, thiadiazole, triazole, indole, benzofuran, benzoxazole, benzothiazole, benzimidazole, benzoxadiazole, benzothiadiazole, benzotriazole, oxazolopyridine, pyrazolopyridine, imidazopyridine, pyrrolopyridine, pyrrolopyrimidine, indolizine, purine, furopyridine, thienopyridine, furopyrrole, furofuran, thienofuran, 1,4-dihydropyrrolopyrrole, thienopyrrole, thienothiophene, quinoline, isoquinoline, furopyrazole, thienopyrazole, and 1,6-dihydropyrrolopyrazole. In a further embodiment is a compound of Formula (XIa) wherein R⁸ is a bond. In yet a further embodiment is a compound of Formula (XIa) wherein R⁷ is a linear or branched alkyl chain of 1-22 carbon atoms, optionally comprising within the alkyl chain or at an alkyl chain terminus an optionally substituted aryl, an optionally substituted heteroaryl, an optionally substituted heterocycloalkyl, or an optionally substituted

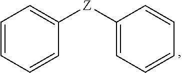

wherein Z is a bond, O, S, NH, CH₂, NHCH₂, or C≡C. In yet a further embodiment is a compound of Formula (XIa) wherein R⁷ is a linear or branched alkyl chain of 1-22 carbon atoms, optionally comprising within the alkyl chain or at an alkyl chain terminus an optionally substituted

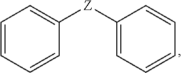

wherein Z is a bond.

In a further embodiment is a compound of Formula (XI′) having the structure of Formula (XIaa):

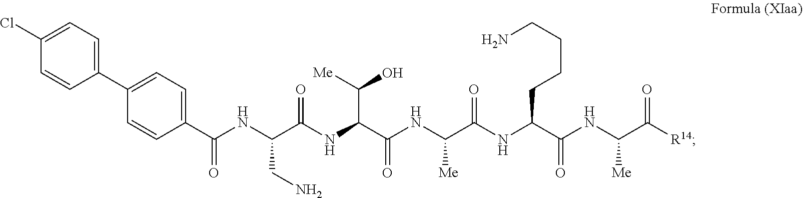

wherein R¹⁴ is an optionally substituted heteroaryl selected from furan, thiophene, pyrrole, pyridine, oxazole, thiazole, imidazole, isoxazole, isothiazole, pyrazole, pyridazine, pyrimidine, pyrazine, oxadiazole, thiadiazole, triazole, indole, benzofuran, benzoxazole, benzothiazole, benzimidazole, benzoxadiazole, benzothiadiazole, benzotriazole, oxazolopyridine, pyrazolopyridine, imidazopyridine, pyrrolopyridine, pyrrolopyrimidine, indolizine, purine, furopyridine, thienopyridine, furopyrrole, furofuran, thienofuran, 1,4-dihydropyrrolopyrrole, thienopyrrole, thienothiophene, quinoline, isoquinoline, furopyrazole, thienopyrazole, and 1,6-dihydropyrrolopyrazole. In another embodiment is a compound of Formula (XIaa) wherein R¹⁴ is an optionally substituted oxazole. In another embodiment is a compound of Formula (XIaa) wherein R¹⁴ is an optionally substituted oxadiazole. In another embodiment is a compound of Formula (XIaa) wherein R¹⁴ is an optionally substituted benzoxazole.

In another embodiment is a compound of Formula (XI) or Formula (XI′) wherein R¹ is

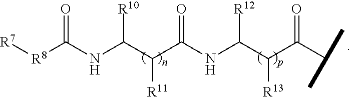

In a further embodiment is a compound of Formula (XI) or Formula (XI′) wherein R⁸ is a bond. In another embodiment is a compound of Formula (XI) or Formula (XI′) wherein R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

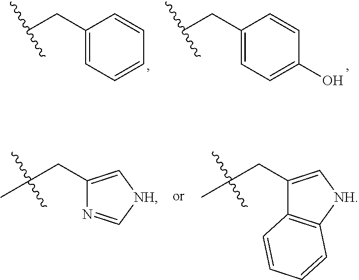

In a further embodiment is a compound of Formula (XI) or Formula (XI′) wherein R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

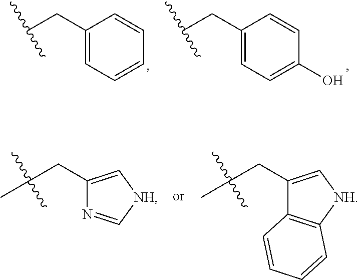

In yet a further embodiment is a compound of Formula (XI) or Formula (XI′) wherein R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂, or

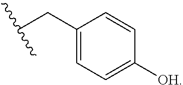 [SMILES: *Cc1ccc(O)cc1]

In a further embodiment of the aforementioned embodiments is a compound of Formula (XI) or Formula (XI′) wherein n is 0 and p is 0. In another embodiment, n is 0 and p is 1. In yet a further embodiment, n is 1 and p is 0.

In a further embodiment is a compound of Formula (XI′) having the structure of Formula (XIb):

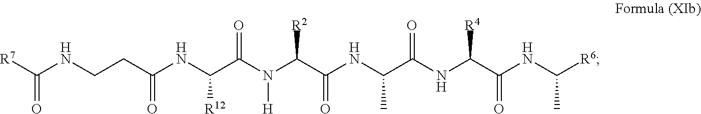

wherein R², R⁴, and R¹², are each independently —CH₂CH(CH₃)₂, —(CH₂)₃NH₂, or —(CH₂)₄NH₂.

In another embodiment is a compound of Formula (XIb) wherein R², R⁴, and R¹² are each —(CH₂)₄NH₂. In another embodiment is a compound of Formula (XIb) wherein R², R⁴, and R¹² are each —(CH₂)₃NH₂. In another embodiment is a compound of Formula (XIb) wherein R⁴ is —CH₂CH(CH₃)₂, R² is —(CH₂)₃NH₂, and R¹² is —(CH₂)₄NH₂. In another embodiment is a compound of Formula (XIb) wherein R⁴ is —CH₂CH(CH₃)₂, R² is —(CH₂)₄NH₂, and R¹² is —(CH₂)₄NH₂.

In a further embodiment is a compound of Formula (XI′) having the structure of Formula (XIbb):

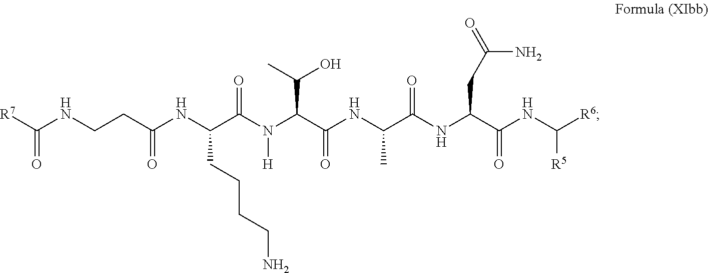

wherein R⁵ is —H, or —CH₃.

In a further embodiment is a compound of Formula (XI′) having the structure of Formula (XIbbb):

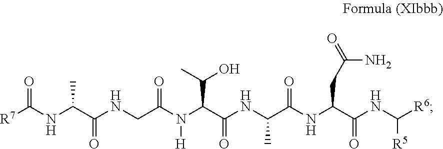

wherein R⁵ is —H, or —CH₃.

In another embodiment is a compound of Formula (XI) or Formula (XI′) wherein R¹ is

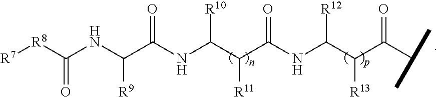

In a further embodiment, R⁸ is a bond. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, (CH₂)₄NH₂,

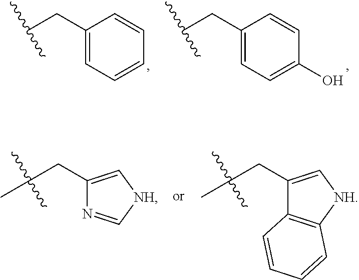

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

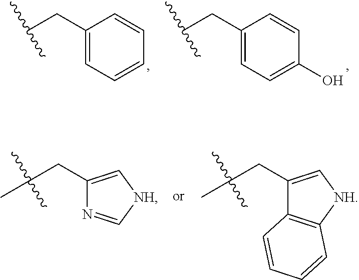

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

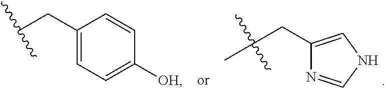 [SMILES: *Cc1ccc(O)cc1 ; "or" ; *Cc1c[nH]cn1 ; "."]

In a further embodiment is a compound of Formula (XI′) having the structure of Formula (XIc):

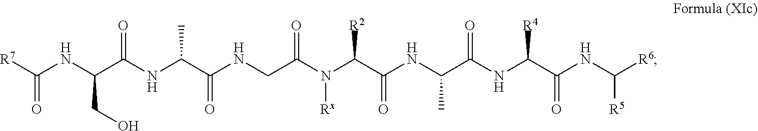

In another embodiment is a compound of Formula (XIc) wherein R² is —CH(OH)(CH₃), —CH₂CH₂C(O)OH, or —(CH₂)₄NH₂. In some embodiments, R² is —CH(OH)(CH₃). In some embodiments, R² is —CH₂CH₂C(O)OH. In some embodiments, R² is —(CH₂)₄NH₂. In a further embodiment is a compound of Formula (XIc) wherein R⁴ is CH₂CH(CH₃)₂ or —CH₂C(O)NH₂. In some embodiments, R⁴ is CH₂CH(CH₃)₂. In some embodiments, R⁴ is —CH₂C(O)NH₂. In yet a further embodiment is a compound of Formula (XIc) wherein R⁵ is H or —CH₃. In some embodiments, R⁴ is H. In some embodiments, R⁴ is —CH₃.

In another embodiment is a compound of Formula (XI) or Formula (XI′) wherein R¹ is

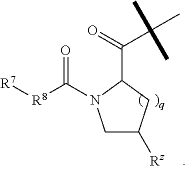

In a further embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

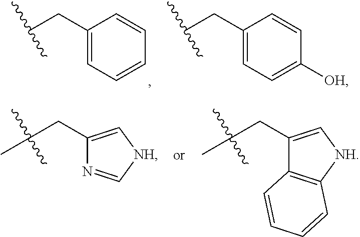

In a further embodiment, q is 1 and R⁸ is a bond.

In a further embodiment is a compound of Formula (XI′) having the structure of Formula (XId):

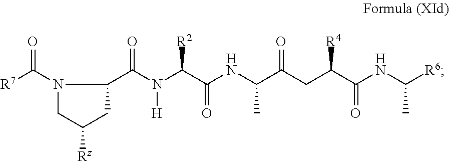

wherein R^{z} is NH₂; and R² and R⁴ are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (XId) wherein R² is —CH(OH)(CH₃), and R⁴ is —CH₂C(O)NH₂. In another embodiment is a compound of Formula (XId) wherein R² is —CH(OH)(CH₃), and R⁴ is —(CH₂)₂NH₂. In another embodiment is a compound of Formula (XId) wherein R² is —CH(OH)(CH₃), and R⁴ is —(CH₂)₃NH₂. In another embodiment is a compound of Formula (XId) wherein R² is —CH(OH)(CH₃), and R⁴ is —(CH₂)₄NH₂. In another embodiment is a compound of Formula (XId) wherein R² is —(CH₂)₄NH₂ and R⁴ is —CH₂CH(CH₃)₂. In another embodiment is a compound of Formula (XId) wherein R² is —(CH₂)₄NH₂ and R⁴ is —CH₂C(O)NH₂. In another embodiment is a compound of Formula (XId) wherein R² is —(CH₂)₄NH₂ and R⁴ is —(CH₂)₄NH₂.

In another embodiment is a compound of Formula (XI) or Formula (XI′) wherein R¹ is

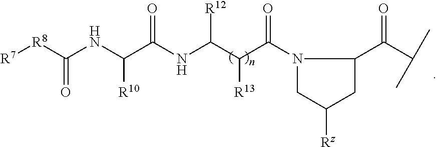

In a further embodiment, R⁸ is a bond. In another embodiment, R², R⁴, R¹⁰, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

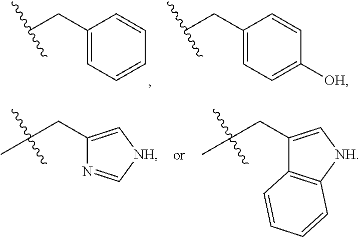

In a further embodiment, R², R⁴, R¹⁰, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

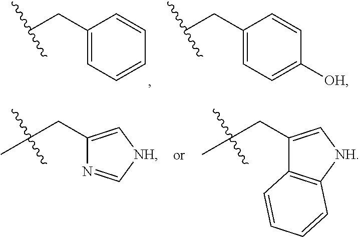

In yet a further embodiment, R², R⁴, R¹⁰, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

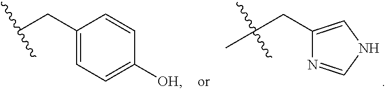 [SMILES: *Cc1ccc(O)cc1 ; *Cc1c[nH]cn1]

In a further embodiment of the aforementioned embodiments is a compound of Formula (XI) or Formula (XI′) wherein n is 0. In yet a further embodiment, n is 1.

In a further embodiment is a compound of Formula (XI′) having the structure of Formula (XIdd):

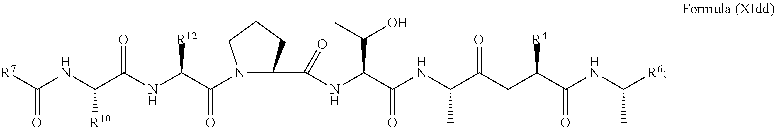

wherein R⁵ is —H, or —CH₃.

In another embodiment is a compound of Formula (XIdd) wherein R¹⁰ is —CH₂OH, and R¹² is —CH₃. In another embodiment is a compound of Formula (XIdd) wherein R¹⁰ is —CH₂CH(CH₃)₂, and R¹² is —CH(OH)(CH₃). In another embodiment of the aforementioned compounds of Formula (XId) is a compound wherein R⁴ is —CH₂C(O)NH₂. In yet another embodiment of the aforementioned compounds of Formula (XIdd) is a compound wherein R⁴ is

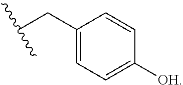 [SMILES: *Cc1ccc(O)cc1]

In another embodiment is a compound of Formula (XI) or Formula (XI′) wherein R¹ is

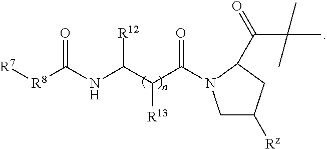

In a further embodiment, R⁸ is a bond. In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

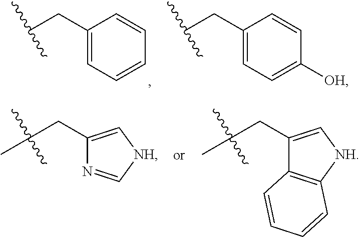

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

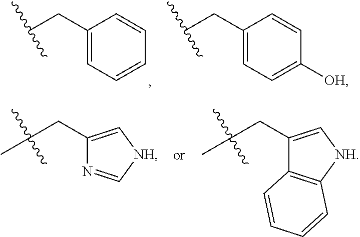

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂NH₂, —CH₂CH₂C(O)NH₂, —(CH₂)₂NH₂, —(CH₂)₃NH₂, —(CH₂)₄NH₂,

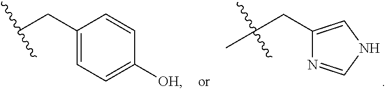 [SMILES: *Cc1ccc(O)cc1 ; *Cc1c[nH]cn1]

In a further embodiment of the aforementioned embodiments is a compound of Formula (XI) or Formula (XI′) wherein n is 0. In yet a further embodiment, n is 1.

In another embodiment is a compound of Formula (XI) or Formula (XI′) wherein R¹ is

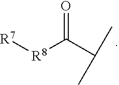

In a further embodiment, R⁸ is a bond. In another embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

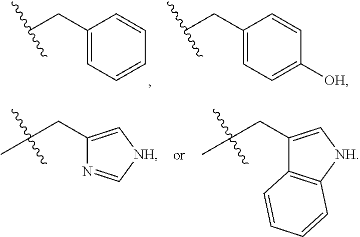

In a further embodiment, R² and R⁴ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

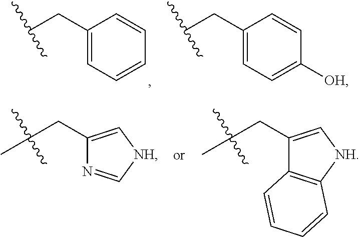

In yet a further embodiment, R² and R⁴ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

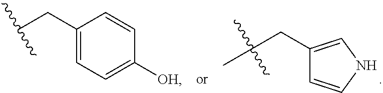 [SMILES: *Cc1ccc(O)cc1 ; *Cc1cc[nH]c1]

In another embodiment is a compound of Formula (XI) or Formula (XI′) wherein R^{x} and R² together with the nitrogen atom form an optionally substituted nitrogen containing ring. In a further embodiment is a compound of Formula (XI′) having the structure of Formula (XIe):

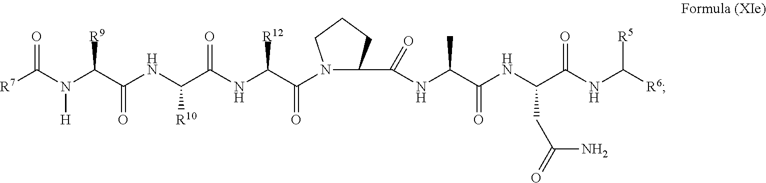

wherein R⁵ is —H, or —CH₃.

In another embodiment is a compound of Formula (XIe) wherein R¹⁰ and R¹² are each independently —H, —CH₃, —CH₂CH(CH₃)₂, —CH₂OH, or —CH(OH)(CH₃).

In another aspect described herein are compounds of Formula (XII):

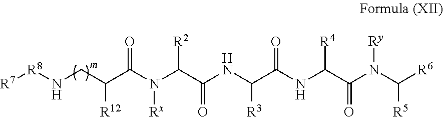

wherein:
R², R⁴, and R¹² are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —NR²¹R²², —CH₂NR²¹R²², —(CH₂)₂NR²¹R²², —(CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

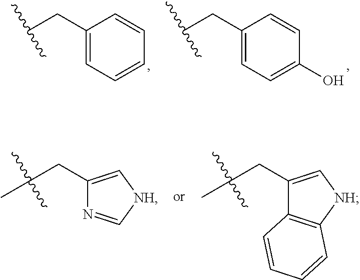

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

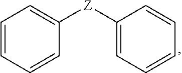

wherein Z is a bond, O, S, NH, CH₂, NHCH₂, or C≡C;
R⁸ is a bond, C(O), optionally substituted C₁-C₆alkyl, optionally substituted C₁-C₆heteroalkyl, optionally substituted aryl, optionally substituted heteroaryl, or optionally substituted heterocycloalkyl;
R¹⁴ is optionally substituted heteroaryl;
each R²¹ is independently H, or C₁-C₄alkyl;
each R²² is independently H, C₁-C₄alkyl, —C(O)R²⁸, —C(═NH)(NH₂), or —CH(═NH);
R²⁸ is H, or optionally substituted C₁-C₆alkyl; and
m is 0-4;
or a pharmaceutically acceptable salt, solvate, or prodrug thereof

In another embodiment is a compound of Formula (XII) having the structure of Formula (XII′):

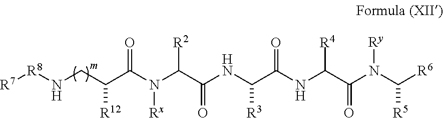

wherein:
R², R⁴, and R¹² are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —NR²¹R²², —CH₂NR²¹R²², —(CH₂)₂NR²¹R²², —(CH₂)₃NR²¹R²², —CH₂ optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

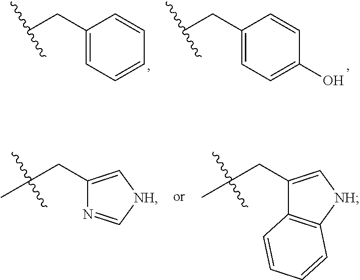

R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

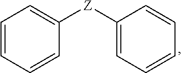

wherein Z is a bond, O, S, NH, CH₂, NHCH₂, or C≡C;
R⁸ is a bond, C(O), optionally substituted C₁-C₆alkyl, optionally substituted C₁-C₆heteroalkyl, optionally substituted aryl, optionally substituted heteroaryl, or optionally substituted heterocycloalkyl;
R¹⁴ is optionally substituted heteroaryl;
each R²¹ is independently H, or C₁-C₄alkyl;
each R²² is independently H, C₁-C₄alkyl, —C(O)R²⁸, —C(═NH)(NH₂), or —CH(═NH); and
R²⁸ is H, or optionally substituted C₁-C₆alkyl;
or a pharmaceutically acceptable salt, solvate, or prodrug thereof

In another embodiment is a compound of Formula (XII) or Formula (XII′) wherein R⁸ is a bond. In another embodiment is a compound of Formula (XII) or Formula (XII′) wherein R⁸ is C(O). In another embodiment is a compound of Formula (XII) or Formula (XII′) wherein R⁸ is an optionally substituted C₁-C₆alkyl. In another embodiment is a compound of Formula (XII) or Formula (XII′) wherein R⁸ is an optionally substituted C₁-C₆heteroalkyl.

In another embodiment of Formula (XII) or Formula (XII′), R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

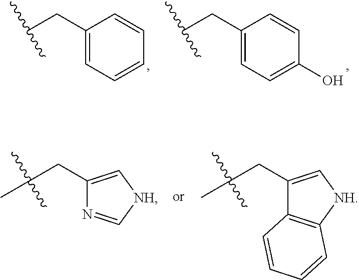

In a further embodiment, R² and R⁴ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

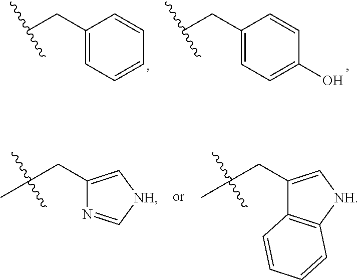

In yet a further embodiment, R² and R⁴ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

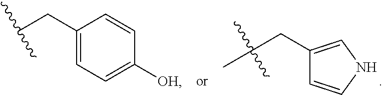 [SMILES: *Cc1ccc(O)cc1 ; *Cc1cc[nH]c1]

In another embodiment is a compound of Formula (XII) or Formula (XII′) wherein R¹² is —H, —CH₂OH, —CH(OH)(CH₃), —CH₂C(O)OH, —CH₂CH₂C(O)OH, —CH₂C(O)NH₂, —CH₂CH₂C(O)NH₂, —NR²¹R²², —CH₂NR²¹R²², —(CH₂)₂NR²¹R²², —(CH₂)₃NR²¹R²², or —(CH₂)₄NR²¹R²². In another embodiment is a compound of Formula (XII) or Formula (XII′) wherein R¹² is —H, —NR²¹R²², —CH₂NR²¹R²², —(CH₂)₂NR²¹R²², —(CH₂)₃NR²¹R²², or —(CH₂)₄NR²¹R²². In another embodiment is a compound of Formula (XII) or Formula (XII′) wherein R¹² is —H, —NR²¹R²², —CH₂NR²¹R²², —(CH₂)₂NR²¹R²². In another embodiment is a compound of Formula (XII) or Formula (XII′) wherein R¹² is —H. In another embodiment is a compound of Formula (XII) or Formula (XII′) wherein R¹² is —NR²¹R²². In another embodiment is a compound of Formula (XII) or Formula (XII′) wherein R¹² is —NH₂. In another embodiment is a compound of Formula (XII) or Formula (XII′) wherein R¹² is —N(H)C(O)CH₃. In another embodiment is a compound of Formula (XII) or Formula (XII′) wherein R¹² is —CH₂NH₂. In another embodiment is a compound of Formula (XII) or Formula (XII′) wherein R¹² is —(CH₂)₂NH₂.

In another embodiment is a compound of Formula (XII) or Formula (XII′) wherein R⁴ is —(CH₂)₄NH₂, R² is —CH(OH)(CH₃), and R¹² is —(CH₂)₂NH₂. In another embodiment is a compound of Formula (XII) or Formula (XII′) wherein R⁴ is —(CH₂)₄NH₂, R² is —CH(OH)(CH₃), and R¹² is —CH₂NH₂. In another embodiment is a compound of Formula (XII) or Formula (XII′) wherein R⁴ is —CH₂C(O)NH₂, R² is —CH(OH)(CH₃), and R¹² is —(CH₂)₄NH₂. In another embodiment is a compound of Formula (XII) or Formula (XII′) wherein R⁴ is —(CH₂)₄NH₂, R² is —(CH₂)₄NH₂, and R¹² is —CH₂NH₂. In another embodiment is a compound of Formula (XII) or Formula (XII′) wherein R⁴ is —CH₂C(O)NH₂, R² is —(CH₂)₄NH₂, and R¹² is —CH₂NH₂. In another embodiment is a compound of Formula (XII) or Formula (XII′) wherein R⁴ is —CH₂CH(CH₃)₂, R² is —(CH₂)₂NH₂, and R¹² is —(CH₂)₂NH₂. In another embodiment of the aforementioned embodiments of Formula (XII) or Formula (XII′) is a compound wherein R¹⁴ is an optionally substituted heteroaryl selected from furan, thiophene, pyrrole, pyridine, oxazole, thiazole, imidazole, isoxazole, isothiazole, pyrazole, pyridazine, pyrimidine, pyrazine, oxadiazole, thiadiazole, triazole, indole, benzofuran, benzoxazole, benzothiazole, benzimidazole, benzoxadiazole, benzothiadiazole, benzotriazole, oxazolopyridine, pyrazolopyridine, imidazopyridine, pyrrolopyridine, pyrrolopyrimidine, indolizine, purine, furopyridine, thienopyridine, furopyrrole, furofuran, thienofuran, 1,4-dihydropyrrolopyrrole, thienopyrrole, thienothiophene, quinoline, isoquinoline, furopyrazole, thienopyrazole, and 1,6-dihydropyrrolopyrazole. In a further embodiment is a compound of Formula (XII) or Formula (XII′) wherein R⁸ is a bond. In yet a further embodiment is a compound of Formula (XII) or Formula (XII′) wherein R⁷ is a linear or branched alkyl chain of 1-22 carbon atoms, optionally comprising within the alkyl chain or at an alkyl chain terminus an optionally substituted aryl, an optionally substituted heteroaryl, an optionally substituted heterocycloalkyl, or an optionally substituted

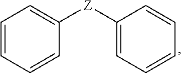

wherein Z is a bond, O, S, NH, CH₂, NHCH₂, or C≡C. In yet a further embodiment is a compound of Formula (XII) or Formula (XII′) wherein R⁷ is a linear or branched alkyl chain of 1-22 carbon atoms, optionally comprising within the alkyl chain or at an alkyl chain terminus an optionally substituted

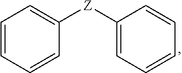

wherein Z is a bond.

In another aspect described herein are compounds of Formula (XIII):

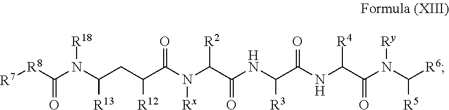

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², (CH₂)₃NR²¹R²², —(CH₂)₄NR²¹R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

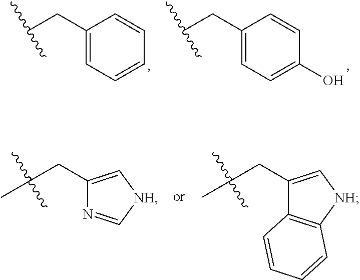

R¹² and R¹³ are each independently —H, —NR²¹R²², —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, or optionally substituted C₁-C₈heteroalkyl; or R¹² and R¹³ together with the carbon atoms to which they are attached form a heterocycloalkyl ring;
R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

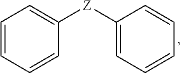

wherein Z is a bond, O, S, NH, CH₂, NHCH₂, or C≡C;
R⁸ is a bond, optionally substituted aryl, optionally substituted heteroaryl, or optionally substituted heterocycloalkyl;
R¹⁴ is optionally substituted heteroaryl;
R¹⁸ is H, or methyl; or R¹⁸ and R¹² together with the atoms to which they are attached form a heterocycloalkyl ring;
each R²¹ is independently H, or C₁-C₄alkyl;
each R²² is independently H, C₁-C₄alkyl, —C(O)R²⁸, —C(═NH)(NH₂), or —CH(═NH);
R²⁸ is H, or optionally substituted C₁-C₆alkyl; and
m is 0-4;
or a pharmaceutically acceptable salt, solvate, or prodrug thereof

In another embodiment is a compound of Formula (XIII) having the structure of Formula (XIII′):

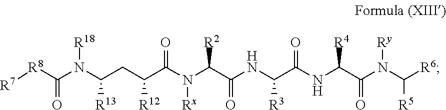

wherein:
R² and R⁴ are each independently —H, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², (CH₂)₃NR²¹R²², —(CH₂)₄NR²¹, R²², optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

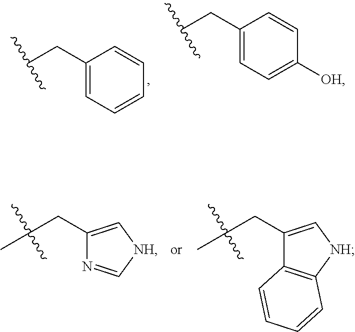 [SMILES: *Cc1ccccc1 ; *Cc1ccc(O)cc1 ; *Cc1c[nH]cn1 ; *Cc1c[nH]c2ccccc12]

R¹² and R¹³ are each independently —H, —NR²¹R²², —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², —(CH₂)₄NR²¹R²², optionally substituted C₁-C₈alkyl, or optionally substituted C₁-C₈heteroalkyl; or R¹² and R¹³ together with the carbon atoms to which they are attached form a heterocycloalkyl ring;
R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R⁷ is optionally substituted aryl, optionally substituted heteroaryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heteroaryl, an optionally substituted heterocycloalkyl, or an optionally substituted

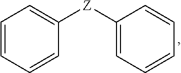

wherein Z is a bond, O, S, NH, CH₂, NHCH₂, or C≡C;
R⁸ is a bond, optionally substituted aryl, optionally substituted heteroaryl, or optionally substituted heterocycloalkyl;
R¹⁴ is optionally substituted heteroaryl;
R¹⁸ is H, or methyl; or R¹⁸ and R¹² together with the atoms to which they are attached form a heterocycloalkyl ring;
each R²¹ is independently H, or C₁-C₄alkyl;
each R²² is independently H, C₁-C₄alkyl, —C(O)R²⁸, —C(═NH)(NH₂), or —CH(═NH);
R²⁸ is H, or optionally substituted C₁-C₆alkyl, and
m is 0-4;
   or a pharmaceutically acceptable salt, solvate, or prodrug thereof

In some embodiments is a compound of Formula (XIII) or Formula (XIII′) wherein R⁸ is a bond. In a further embodiment is a compound of Formula (XIII) or Formula (XIII′) wherein R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²². In yet a further embodiment is a compound of Formula (XIII) or Formula (XIII′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)OH, —CH₂CH₂C(O)OH, —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment is a compound of Formula (XIII) or Formula (XIII′) wherein R¹² is —H, —CH₂OH, —CH(OH)(CH₃), —CH₂C(O)OH, —CH₂CH₂C(O)OH, —CH₂C(O)NH₂, —CH₂CH₂C(O)NH₂, —NR²¹R²², —CH₂NR²¹R²², —(CH₂)₂NR²¹R²², —(CH₂)₃NR²¹R²², or —(CH₂)₄NR²¹R²². In another embodiment is a compound of Formula (XIII) or Formula (XIII′) wherein R¹² is —H, —NR²¹R²², —CH₂NR²¹R²², —(CH₂)₂NR²¹R²², —(CH₂)₃NR²¹R²², or —(CH₂)₄NR²¹R²². In another embodiment is a compound of Formula (XIII) or Formula (XIII′) wherein R¹² is —H, —NR²¹R²², —CH₂NR²¹R²², —(CH₂)₂NR²¹R²². In another embodiment is a compound of Formula (XIII) or Formula (XIII′) wherein R¹² is —H. In another embodiment is a compound of Formula (XIII) or Formula (XIII′) wherein R¹² is —NR²¹R²². In another embodiment is a compound of Formula (XIII) or Formula (XIII′) wherein R¹² is —NH₂. In another embodiment is a compound of Formula (XIII) or Formula (XIII′) wherein R¹² is —N(H)C(O)CH₃. In another embodiment is a compound of Formula (XIII) or Formula (XIII′) wherein R¹² is —CH₂NH₂. In another embodiment is a compound of Formula (XIII) or Formula (XIII′) wherein R¹² is —(CH₂)₂NH₂.

In another embodiment is a compound of Formula (XIII) or Formula (XIII′) wherein R¹² and R¹³ together with the carbon atoms to which they are attached form a heterocycloalkyl ring. In a further embodiment is a compound of Formula (XIII) or Formula (XIII′) wherein R¹² and R¹³ together with the carbon atoms to which they are attached form a pyrrolidine ring. In yet a further embodiment is a compound of Formula (XIII) or Formula (XIII′) wherein R² and R⁴ are each independently —CH(CH₃)₂, —CH(CH₃)(CH₂CH₃), —CH₂CH(CH₃)₂, —CH₂OH, —CH(OH)(CH₃), —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment is a compound of Formula (XIII) or Formula (XIII′) wherein R¹⁸ and R¹² together with the atoms to which they are attached form a heterocycloalkyl ring. In a further embodiment is a compound of Formula (XIII) or Formula (XIII′) wherein R¹⁸ and R¹² together with the atoms to which they are attached form a piperidine ring In yet a further embodiment is a compound of Formula (XIII) or Formula (XIII′) wherein R¹³ is H and R² and R⁴ are each independently —CH(CH₃)₂, —CH(CH₃)(CH₂CH₃), —CH₂CH(CH₃)₂, —CH₂OH, —CH(OH)(CH₃), —CH₂C(O)NH₂, —CH₂CH₂C(O)NH₂, —CH₂NR²¹R²², —(CH₂)₂NR²¹R²², —(CH₂)₃NR²¹R²², or —(CH₂)₄NR²¹R²².

In another embodiment of the aforementioned embodiments of Formula (XI), (XII), or (XIII) is a compound wherein R⁷ is a linear or branched alkyl chain of about 1-22 carbon atoms. In another embodiment of the aforementioned embodiments of Formula (XI), (XII), or (XIII) is a compound wherein R⁷ is

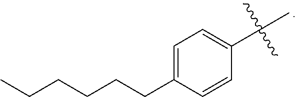 [SMILES: *c1ccc(CCCCCC)cc1]

In another embodiment of the aforementioned embodiments of Formula (XI), (XII), or (XIII) is a compound wherein R⁷ is

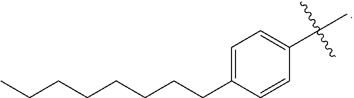 [SMILES: *c1ccc(CCCCCCCC)cc1]

In another embodiment of the aforementioned embodiments of Formula (XI), (XII), or (XIII) is a compound wherein R⁷ is

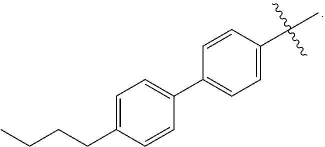 [SMILES: *c1ccc(-c2ccc(CCCC)cc2)cc1]

In another embodiment of the aforementioned embodiments of Formula (XI), (XII), or (XIII) is a compound wherein R⁷ is

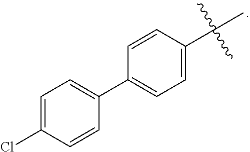 [SMILES: *c1ccc(-c2ccc(Cl)cc2)cc1]

In another embodiment of the aforementioned embodiments of Formula (XI), (XII), or (XIII) is a compound wherein R⁵ is H. In another embodiment of the aforementioned embodiments of Formula (XI), (XII), or (XIII) is a compound wherein R⁵ is methyl. In another embodiment of the aforementioned embodiments of Formula (XI), (XII), or (XIII) is a compound wherein R⁵ is —CH₂OH.

In another embodiment of the aforementioned embodiments of Formula (XI′), (XII′), or (XIII′) is a compound wherein R⁷ is a linear or branched alkyl chain of about 1-22 carbon atoms. In another embodiment of the aforementioned embodiments of Formula (XI′), (XII′), or (XIII′) is a compound wherein R⁷ is

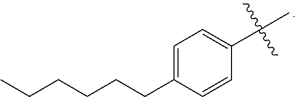 [SMILES: *c1ccc(CCCCCC)cc1]

In another embodiment of the aforementioned embodiments of Formula (XI′), (XII′), or (XIII′) is a compound wherein R⁷ is

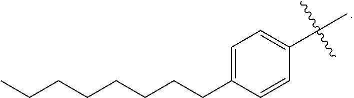 [SMILES: *c1ccc(CCCCCCCC)cc1]

In another embodiment of the aforementioned embodiments of Formula (XI′), (XII′), or (XIII′) is a compound wherein R⁷ is

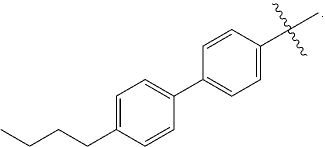 [SMILES: *c1ccc(-c2ccc(CCCC)cc2)cc1]

In another embodiment of the aforementioned embodiments of Formula (XI), (XII), or (XIII) is a compound wherein R⁷ is

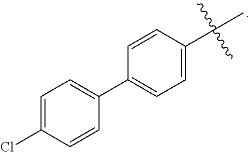 [SMILES: *c1ccc(-c2ccc(Cl)cc2)cc1]

In another embodiment of the aforementioned embodiments of Formula (XI′), (XII′), or (XIII′) is a compound wherein R⁵ is H. In another embodiment of the aforementioned embodiments of Formula (XI′), (XII′), or (XIII′) is a compound wherein R⁵ is methyl. In another embodiment of the aforementioned embodiments of Formula (XI′), (XII′), or (XIII′) is a compound wherein R⁵ is —CH₂OH.

In another aspect is a compound selected from:

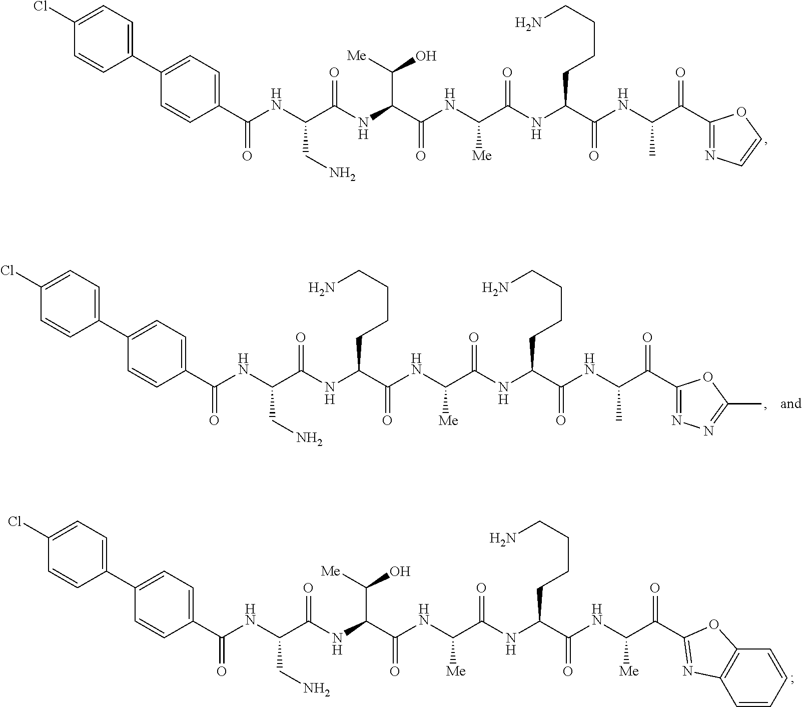 [SMILES: C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)c1ncco1 ; Cc1nnc(C(=O)[C@H](C)NC(=O)[C@H](CCCCN)NC(=O)[C@H](C)NC(=O)[C@H](CCCCN)NC(=O)[C@H](CN)NC(=O)c2ccc(-c3ccc(Cl)cc3)cc2)o1 ; C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)c1nc2ccccc2o1]

or a pharmaceutically acceptable salt, solvate, or prodrug thereof.

In another embodiment is a compound selected from:

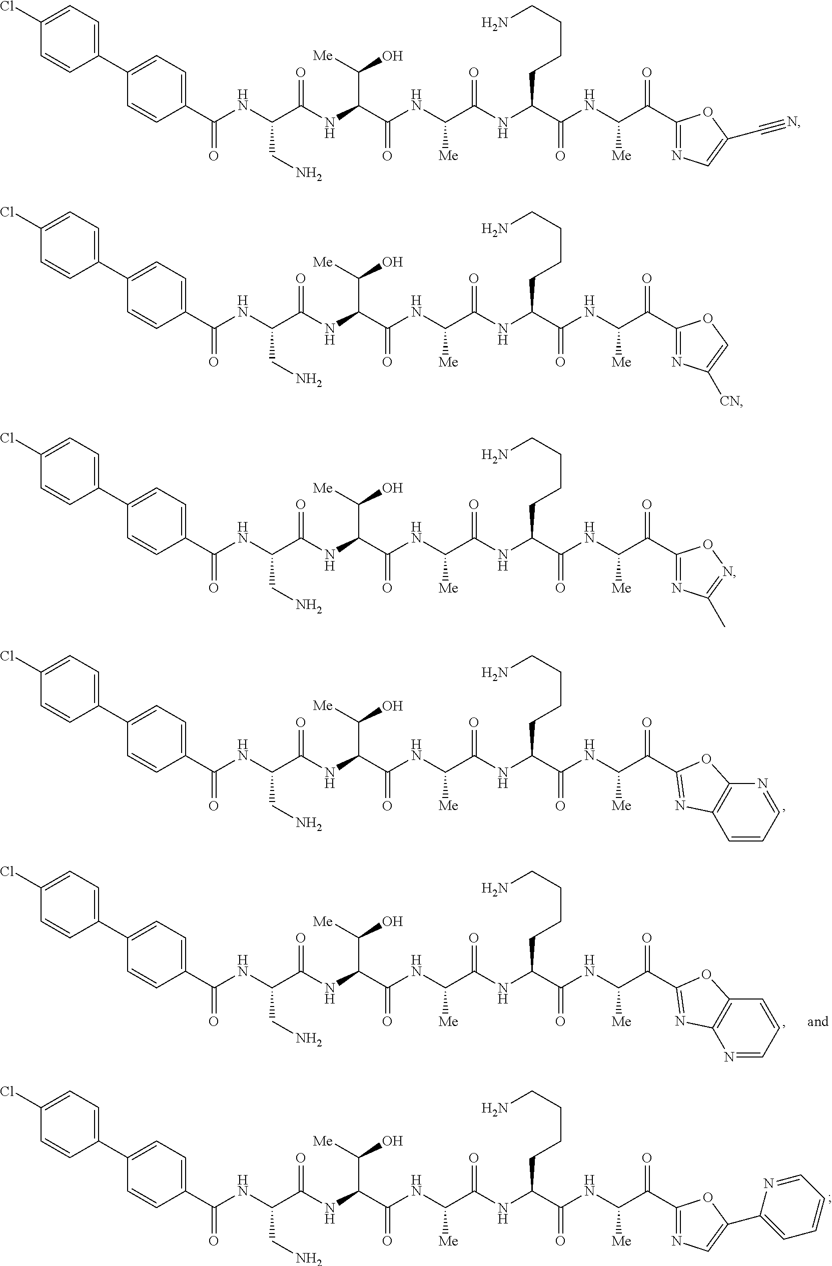

or a pharmaceutically acceptable salt, solvate, or prodrug thereof.

In another aspect described herein are compounds of Formula (XIV):

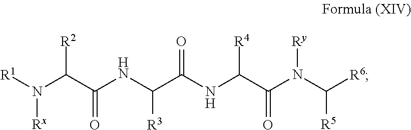

wherein:
R¹ is selected from:

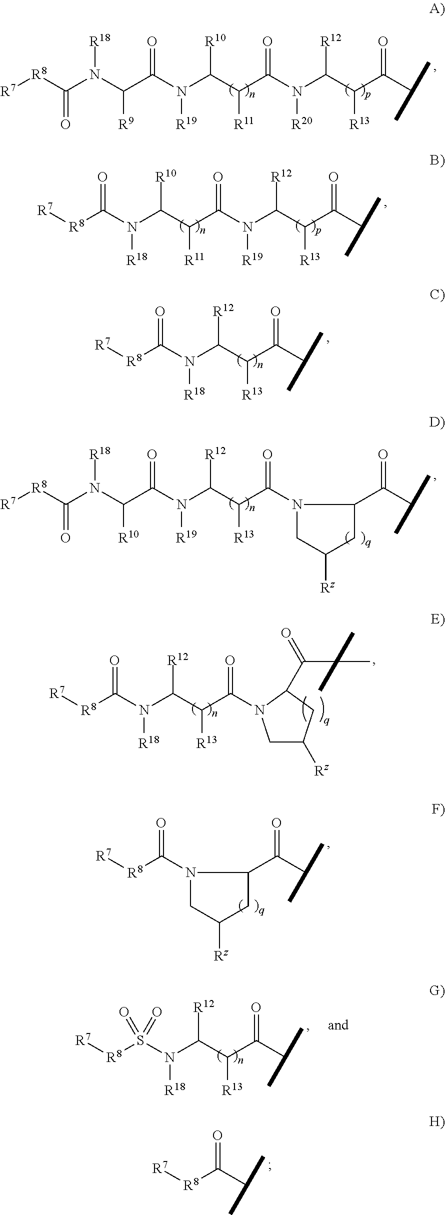

R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

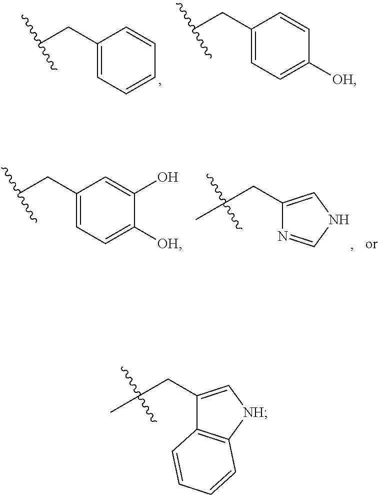

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —C(═O)H, —CH₂C(═O)H, —C(═O)NHCH₂C(═O)H, —C(═O)C(═O)N(R¹⁴)₂, —C(═O)C(═O)OH, —B(OR²³)(OR²⁴), or

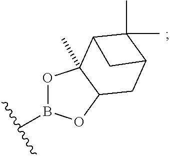 [SMILES: *B1OC2CC3CC(C3(C)C)[C@]2(C)O1]

or R⁵ and R⁶ together with the carbon atom form

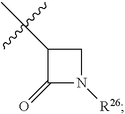

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶; —CH₂—NR¹⁵R¹⁶, or —(CH₂)₂—NR¹⁵R¹⁶;
R⁷ is unsubstituted C₁-C₁₀alkyl;
R⁸ is optionally substituted C₁-C₁₀heteroalkyl;
R⁹ is —CH₂OH, —CH₂CH(CH₃)₂,

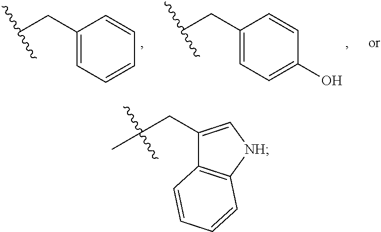 [SMILES: *Cc1ccccc1 ; *Cc1ccc(O)cc1 ; *Cc1c[nH]c2ccccc12]

R¹⁴, R¹⁵, and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH); R²³ and R²⁴ are each independently H, or C₁-C₄alkyl; or R²³ and R²⁴ together with the boron atom form an optionally substituted 5- or 6-membered boron containing ring; each R²⁵ is independently C₁-C₆alkyl;
R²⁶ is H, C₁-C₄alkyl, C₁-C₄alkoxy, —CH₂C(O)OR²⁵, or —OCH₂C(O)OR²⁵;
n is 0 or 1;
p is 0 or 1; and
q is 0 or 1;
   or a pharmaceutically acceptable salt, solvate, or prodrug thereof

In one embodiment is a compound of Formula (XIV) having the structure of Formula (XIV′):

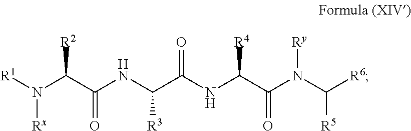

wherein:
R¹ is selected from:

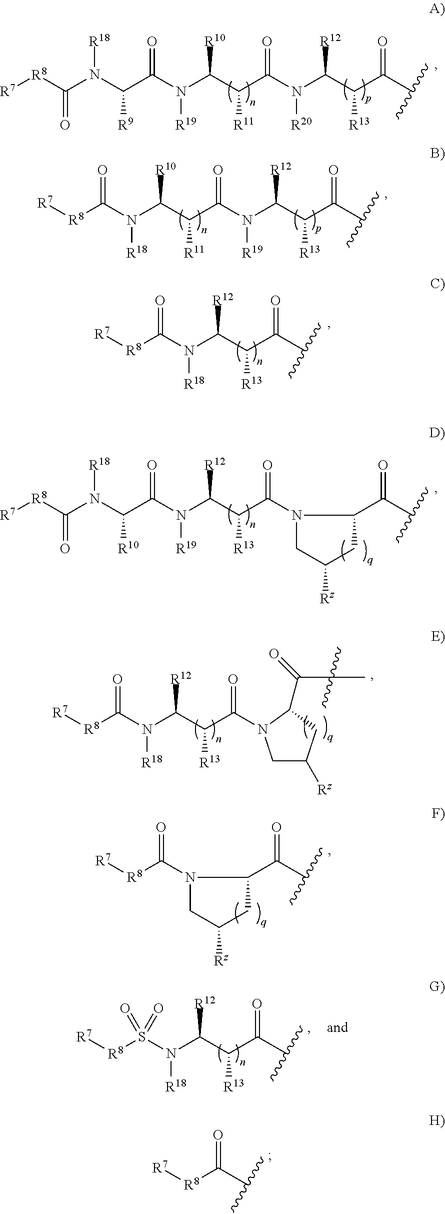

R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

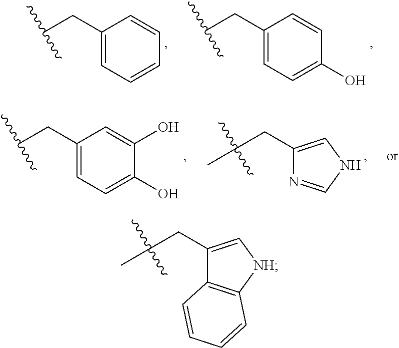

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —C(═O)H, —CH₂C(═O)H, —C(═O)NHCH₂C(═O)H, —C(═O)C(═O)N(R¹⁴)₂, C(═O)C(═O)OH, —B(OR²³)(OR²⁴), or

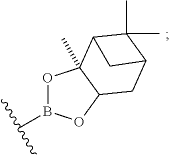 [SMILES: *B1OC2CC3CC(C3(C)C)[C@]2(C)O1]

or R⁵ and R⁶ together with the carbon atom form

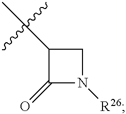

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is unsubstituted C₁-C₁₀alkyl;
R⁸ is optionally substituted C₁-C₁₀heteroalkyl;
R⁹ is —CH₂OH, —CH₂CH(CH₃)₂,

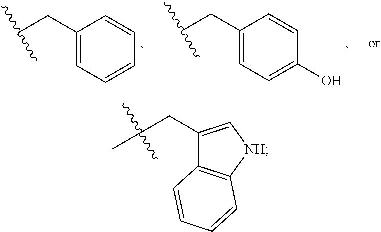 [SMILES: *Cc1ccccc1 ; *Cc1ccc(O)cc1 ; *Cc1c[nH]c2ccccc12]

R¹⁴, R¹⁵, and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ and R²⁴ are each independently H, or C₁-C₄alkyl; or R²³ and R²⁴ together with the boron atom form an optionally substituted 5- or 6-membered boron containing ring;
each R²⁵ is independently C₁-C₆alkyl;
R²⁶ is H, C₁-C₄alkyl, C₁-C₄alkoxy, —CH₂C(O)OR²⁵, or —OCH₂C(O)OR²⁵;
n is 0 or 1;
p is 0 or 1; and
q is 0 or 1;
   or a pharmaceutically acceptable salt, solvate, or prodrug thereof

In another embodiment is a compound of Formula (XIV) or Formula (XIV′) wherein R¹ is

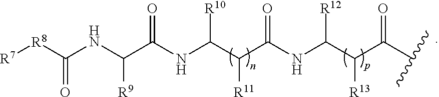

In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

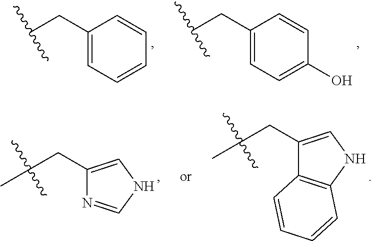

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

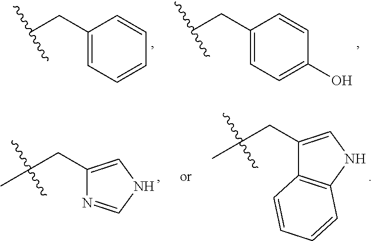

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

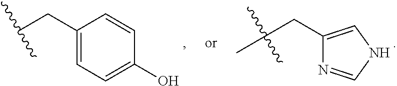 [SMILES: *Cc1ccc(O)cc1 ; *Cc1c[nH]cn1]

In a further embodiment of the aforementioned embodiments is a compound of Formula (XIV) or Formula (XIV′) wherein n is 0 and p is 0. In another embodiment, n is 0 and p is 1. In yet a further embodiment, n is 1 and p is 0.

In a further embodiment is a compound of Formula (XIV′) having the structure of Formula (XIVa):

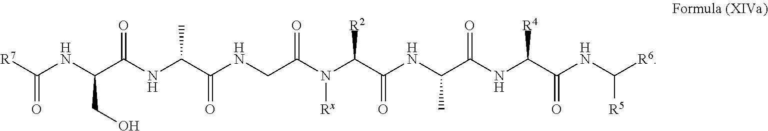

In another embodiment is a compound of Formula (XIVa) wherein R² is —CH(OH)(CH₃), —CH₂CH₂C(O)OH, or —(CH₂)₄NH₂. In some embodiments, R² is —CH(OH)(CH₃). In some embodiments, R² is —CH₂CH₂C(O)OH. In some embodiments, R² is —(CH₂)₄NH₂. In a further embodiment is a compound of Formula (XIVa) wherein R⁴ is CH₂CH(CH₃)₂ or —CH₂C(O)NH₂. In some embodiments, R⁴ is CH₂CH(CH₃)₂. In some embodiments, R⁴ is —CH₂C(O)NH₂. In yet a further embodiment is a compound of Formula (XIVa) wherein R⁵ is H or —CH₃. In some embodiments, R⁴ is H. In some embodiments, R⁴ is —CH₃.

In another embodiment is a compound of Formula (XIV) or Formula (XIV′) wherein R¹ is

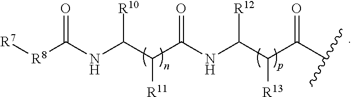

In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

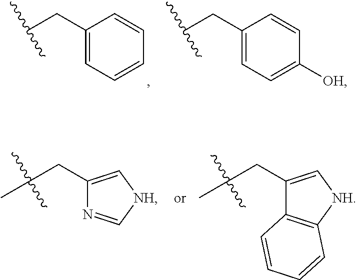

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

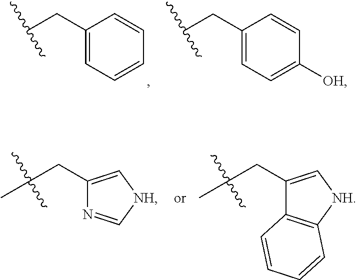

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, CH₂CH₂C(O)NH₂, —CH₂NH₂, —(CH₂)₂NH₂, —(CH₂)₃NH₂, —(CH₂)₄NH₂, or

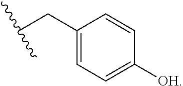 [SMILES: *Cc1ccc(O)cc1]

In a further embodiment of the aforementioned embodiments is a compound of Formula (XIV) or Formula (XIV′) wherein n is 0 and p is 0. In another embodiment, n is 0 and p is 1. In yet a further embodiment, n is 1 and p is 0.

In a further embodiment is a compound of Formula (XIV′) having the structure of Formula (XIVb):

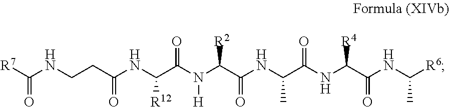

wherein R², R⁴, and R¹², are each independently —CH₂CH(CH₃)₂, —(CH₂)₃NH₂, or —(CH₂)₄NH₂.

In another embodiment is a compound of Formula (XIVb) wherein R², R⁴, and R¹² are each —(CH₂)₄NH₂. In another embodiment is a compound of Formula (XIVb) wherein R², R⁴, and R¹² are each —(CH₂)₃NH₂. In another embodiment is a compound of Formula (XIVb) wherein R⁴ is —CH₂CH(CH₃)₂, R² is —(CH₂)₃NH₂, and R¹² is —(CH₂)₄NH₂. In another embodiment is a compound of Formula (XIVb) wherein R⁴ is —CH₂CH(CH₃)₂, R² is —(CH₂)₄NH₂, and R¹² is —(CH₂)₄NH₂.

In a further embodiment is a compound of Formula (XIV′) having the structure of Formula (XIVbb):

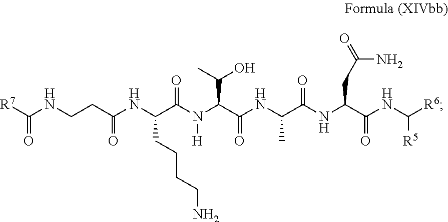

wherein R⁵ is —H, or —CH₃.

In a further embodiment is a compound of Formula (XIV′) having the structure of Formula (XIVbbb):

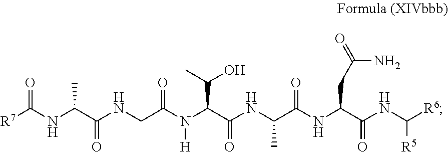

wherein R⁵ is —H, or —CH₃.

In another embodiment is a compound of Formula (XIV) or Formula (XIV′) wherein R¹ is

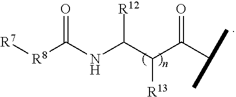

In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

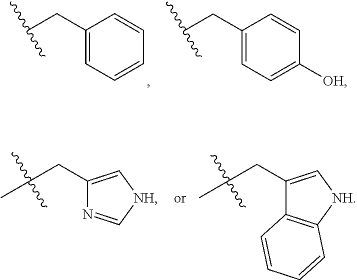

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

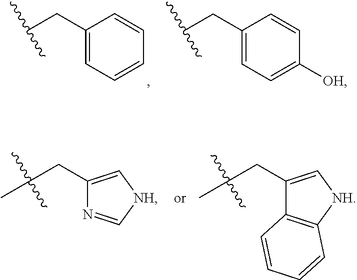

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

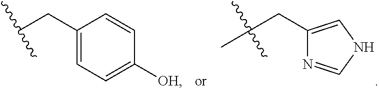 [SMILES: *Cc1ccc(O)cc1 ; *Cc1c[nH]cn1]

In a further embodiment of the aforementioned embodiments is a compound of Formula (XIV) or Formula (XIV′) wherein n is 0. In yet a further embodiment, n is 1.

In a further embodiment is a compound of Formula (XIV′) having the structure of Formula (XIVC):

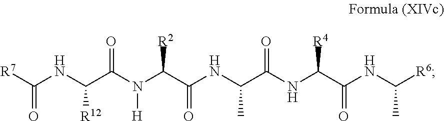

wherein R², R⁴, and R¹², are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (XIVc) wherein R⁴ is —(CH₂)₄NH₂, R² is —CH(OH)(CH₃), and R¹² is —(CH₂)₂NH₂. In another embodiment is a compound of Formula (XIVc) wherein R⁴ is —(CH₂)₄NH₂, R² is —CH(OH)(CH₃), and R¹² is —CH₂NH₂. In another embodiment is a compound of Formula (XIVc) wherein R⁴ is —CH₂C(O)NH₂, R² is —CH(OH)(CH₃), and R¹² is —(CH₂)₄NH₂. In another embodiment is a compound of Formula (XIVc) wherein R⁴ is —(CH₂)₄NH₂, R² is —(CH₂)₄NH₂, and R¹² is —CH₂NH₂. In another embodiment is a compound of Formula (XIVc) wherein R⁴ is —CH₂C(O)NH₂, R² is —(CH₂)₄NH₂, and R¹² is —CH₂NH₂. In another embodiment is a compound of Formula (XIVc) wherein R⁴ is —CH₂CH(CH₃)₂, R² is —(CH₂)₂NH₂, and R¹² is —(CH₂)₂NH₂.

In a further embodiment is a compound of Formula (XIV′) having the structure of Formula (XIVcc):

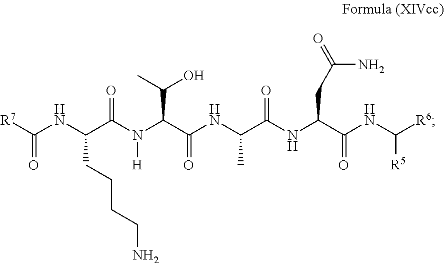

wherein R⁵ is —H, or —CH₃.

In another embodiment is a compound of Formula (XIV) or Formula (XIV′) wherein R¹ is

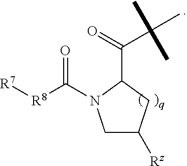

In a further embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

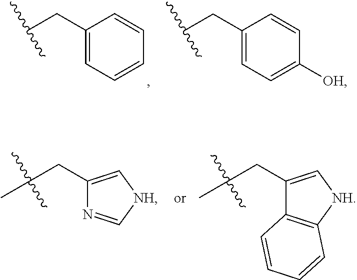

In a further embodiment, q is 1.

In a further embodiment is a compound of Formula (XIV′) having the structure of Formula (XIVd):

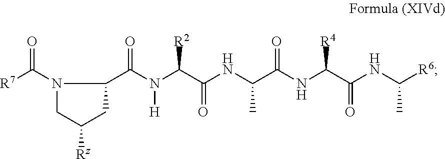

wherein R^{z} is NH₂; and R² and R⁴ are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (XIVd) wherein R² is —CH(OH)(CH₃), and R⁴ is —CH₂C(O)NH₂. In another embodiment is a compound of Formula (XIVd) wherein R² is —CH(OH)(CH₃), and R⁴ is —(CH₂)₂NH₂. In another embodiment is a compound of Formula (XIVd) wherein R² is —CH(OH)(CH₃), and R⁴ is —(CH₂)₃NH₂. In another embodiment is a compound of Formula (XIVd) wherein R² is —CH(OH)(CH₃), and R⁴ is —(CH₂)₄NH₂. In another embodiment is a compound of Formula (XIVd) wherein R² is —(CH₂)₄NH₂ and R⁴ is —CH₂CH(CH₃)₂. In another embodiment is a compound of Formula (XIVd) wherein R² is —(CH₂)₄NH₂ and R⁴ is —CH₂C(O)NH₂. In another embodiment is a compound of Formula (XIVd) wherein R² is —(CH₂)₄NH₂ and R⁴ is —(CH₂)₄NH₂.

In another embodiment is a compound of Formula (XIV) or Formula (XIV′) wherein R¹ is

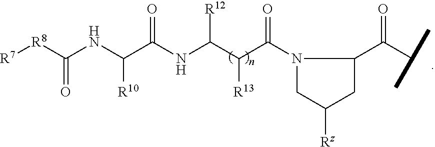

In another embodiment, R², R⁴, R¹⁰, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

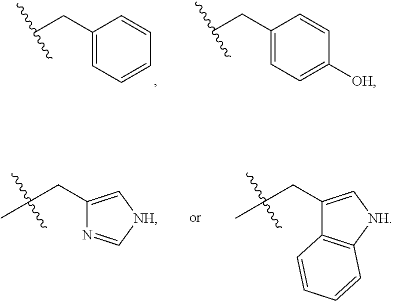 [SMILES: *Cc1ccccc1 ; *Cc1ccc(O)cc1 ; *Cc1c[nH]cn1 ; *Cc1c[nH]c2ccccc12]

In a further embodiment, R², R⁴, R¹⁰, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

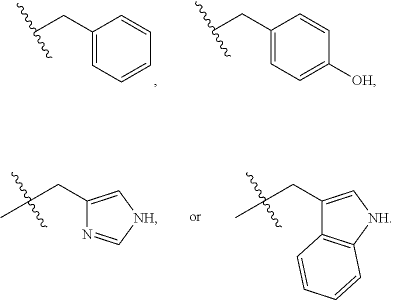 [SMILES: *Cc1ccccc1 ; *Cc1ccc(O)cc1 ; *Cc1c[nH]cn1 ; *Cc1c[nH]c2ccccc12]

In yet a further embodiment, R², R⁴, R¹⁰, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

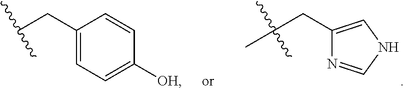 [SMILES: *Cc1ccc(O)cc1 ; *Cc1c[nH]cn1]

In a further embodiment of the aforementioned embodiments is a compound of Formula (XIV) or Formula (XIV′) wherein n is 0. In yet a further embodiment, n is 1.

In a further embodiment is a compound of Formula (XIV′) having the structure of Formula (XIVdd):

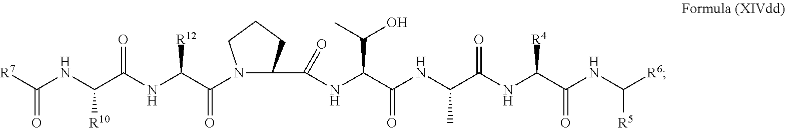

wherein R⁵ is —H, or —CH₃.

In another embodiment is a compound of Formula (XIVdd) wherein R¹⁰ is —CH₂OH, and R¹² is —CH₃. In another embodiment is a compound of Formula (XIVdd) wherein R¹⁰ is —CH₂CH(CH₃)₂, and R¹² is —CH(OH)(CH₃). In another embodiment of the aforementioned compounds of Formula (XIVdd) is a compound wherein R⁴ is —CH₂C(O)NH₂. In yet another embodiment of the aforementioned compounds of Formula (XIVdd) is a compound wherein R⁴ is

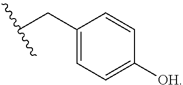 [SMILES: *Cc1ccc(O)cc1]

In another embodiment is a compound of Formula (XIV) or Formula (XIV′) wherein R¹ is

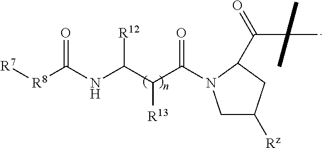

In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

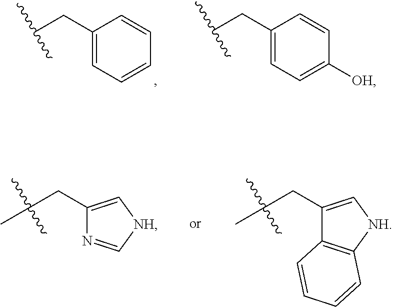 [SMILES: *Cc1ccccc1 ; *Cc1ccc(O)cc1 ; *Cc1c[nH]cn1 ; *Cc1c[nH]c2ccccc12]

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

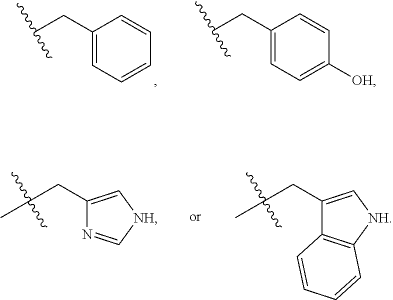 [SMILES: *Cc1ccccc1 ; *Cc1ccc(O)cc1 ; *Cc1c[nH]cn1 ; *Cc1c[nH]c2ccccc12]

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂NH₂, —CH₂CH₂C(O)NH₂, —(CH₂)₂NH₂, —(CH₂)₃NH₂, —(CH₂)₄NH₂,

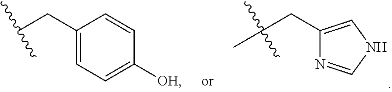 [SMILES: *Cc1ccc(O)cc1 ; *Cc1c[nH]cn1 ; "or"]

In a further embodiment of the aforementioned embodiments is a compound of Formula (XIV) or Formula (XIV′) wherein n is 0. In yet a further embodiment, n is 1.

In another embodiment is a compound of Formula (XIV) or Formula (XIV′) wherein R¹ is

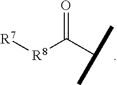

In another embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

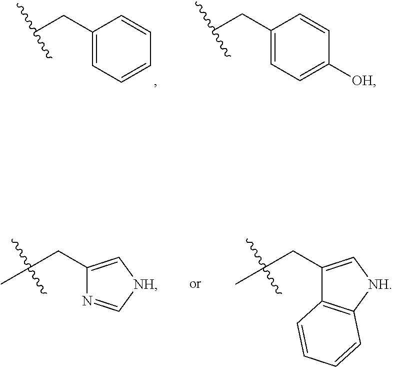 [SMILES: *Cc1ccccc1 ; *Cc1ccc(O)cc1 ; *Cc1c[nH]cn1 ; *Cc1c[nH]c2ccccc12]

In a further embodiment, R² and R⁴ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

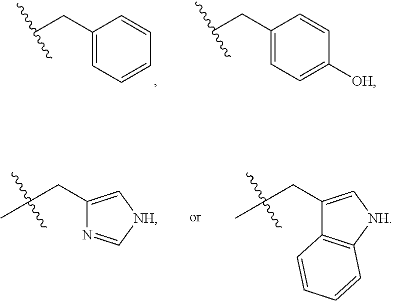 [SMILES: *Cc1ccccc1 ; *Cc1ccc(O)cc1 ; *Cc1c[nH]cn1 ; *Cc1c[nH]c2ccccc12]

In yet a further embodiment, R² and R⁴ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

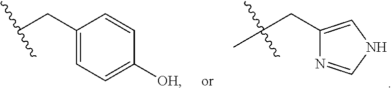 [SMILES: *Cc1ccc(O)cc1 ; *Cc1c[nH]cn1 ; "or"]

In another embodiment is a compound of Formula (XIV) or Formula (XIV′) wherein R^{x} and R² together with the nitrogen atom form an optionally substituted nitrogen containing ring. In a further embodiment is a compound of Formula (XIV′) having the structure of Formula (XIVe):

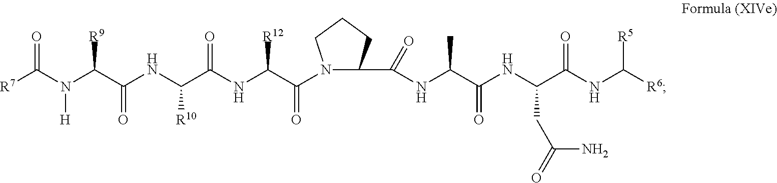

wherein R⁵ is —H, or —CH₃.

In another embodiment is a compound of Formula (XIVe) wherein R¹⁰ and R¹² are each independently —H, —CH₃, —CH₂CH(CH₃)₂, —CH₂OH, or —CH(OH)(CH₃).

In another embodiment of any of the aforementioned embodiments of Formula (XIV) or Formula (XIV′) is a compound wherein R⁶ is

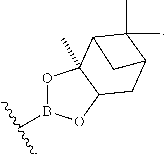 [SMILES: *B1OC2CC3CC(C3(C)C)[C@]2(C)O1]

In another embodiment of any of the aforementioned embodiments of Formula (XIV) or Formula (XIV′) is a compound wherein R⁶ is B(OH)₂. In another embodiment of any of the aforementioned embodiments of Formula (XIV) or Formula (XIV′) is a compound wherein R⁶ is —C(═O)H.

In another embodiment of any of the aforementioned embodiments of Formula (XIV) or Formula (XIV′) is a compound wherein R⁷ is unsubstituted C₁-C₈alkyl. In another embodiment of any of the aforementioned embodiments of Formula (XIV) or Formula (XIV′) is a compound wherein R⁷ is unsubstituted C₁-C₆alkyl.

In another embodiment of any of the aforementioned embodiments of Formula (XIV) or Formula (XIV′) is a compound wherein R⁸ is an optionally substituted C₁-C₈heteroalkyl. In another embodiment of any of the aforementioned embodiments of Formula (XIV) or Formula (XIV′) is a compound wherein R⁸ is an unsubstituted C₁-C₅heteroalkyl. In another embodiment of any of the aforementioned embodiments of Formula (XIV) or Formula (XIV′) is a compound wherein R⁸ is a substituted C₁-C₈heteroalkyl.

In another aspect described herein are compounds of Formula (XV):

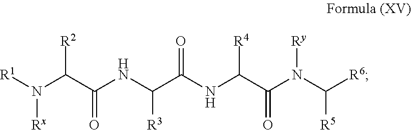

wherein:
R¹ is selected from:

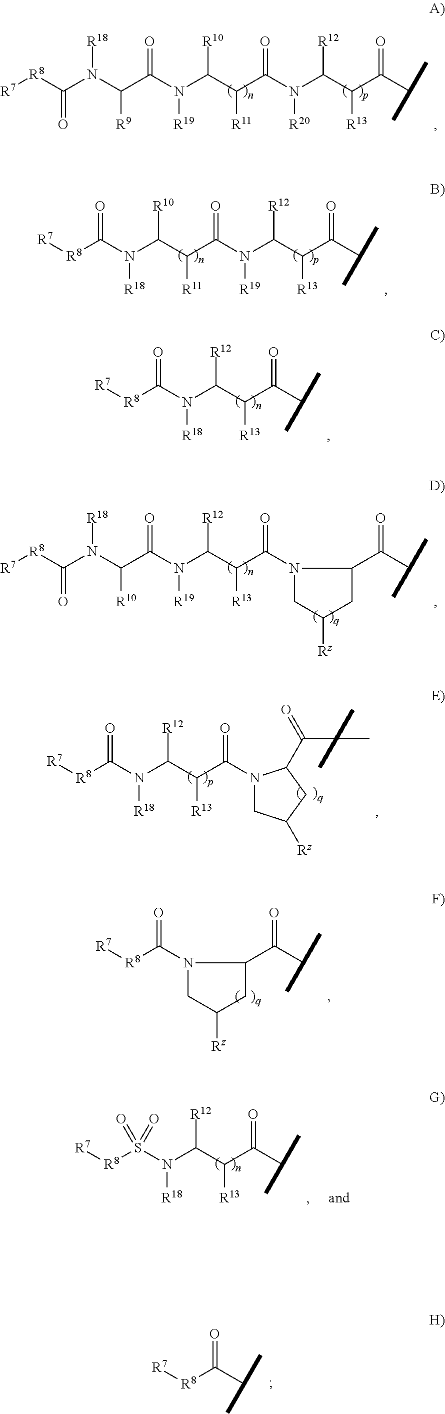

R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

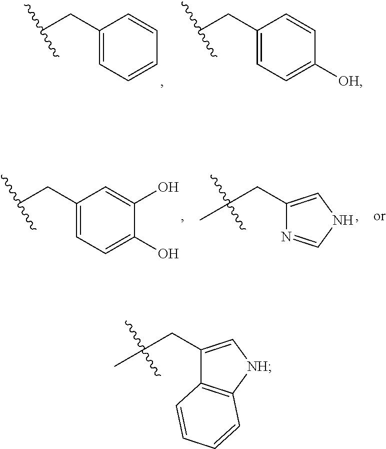

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —C(═O)H, —CH₂C(═O)H, —C(═O)NHCH₂C(═O)H, —C(═O)C(═O)N(R¹⁴)₂, —C(═O)C(═O)OH, —C(═O)R²⁷, —B(OR²³)(OR²⁴), or

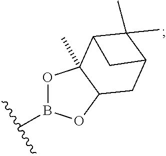 [SMILES: *B1OC2CC3CC(C3(C)C)[C@]2(C)O1]

or R⁵ and R⁶ together with the carbon atom form

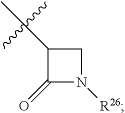

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is

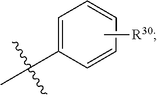

R⁸ is a bond, —O—, or —N(R¹⁷)—, optionally substituted aryl, or optionally substituted heteroaryl;
R⁹ is —CH₂OH, —CH₂CH(CH₃)₂,

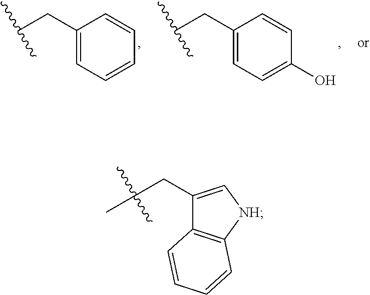

R¹⁴, R¹⁵, and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ and R²⁴ are each independently H, or C₁-C₄alkyl; or R²³ and R²⁴ together with the boron atom form an optionally substituted 5- or 6-membered boron containing ring;
each R²⁵ is independently C₁-C₆alkyl;
R²⁶ is H, C₁-C₄alkyl, C₁-C₄alkoxy, —CH₂C(O)OR²⁵, or —OCH₂C(O)OR²⁵;
R²⁷ is C₁-C₆alkyl, C₁-C₆haloalkyl, optionally substituted heteroaryl, —C(O)OR³², —CF₂C(O)OH, or

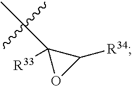

R³⁰ is

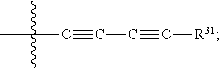

R³¹ is optionally substituted C₁-C₆alkyl;
R³² is optionally substituted C₁-C₆alkyl;
R³³ is H, C₁-C₄alkyl, or C₁-C₄alkoxy;
R³⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

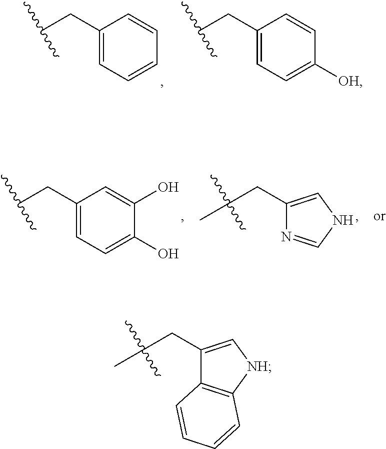

n is 0 or 1;
p is 0 or 1; and
q is 0 or 1;
   or a pharmaceutically acceptable salt, solvate, or prodrug thereof.

In one embodiment is a compound of Formula (XV) having the structure of Formula (XV′):

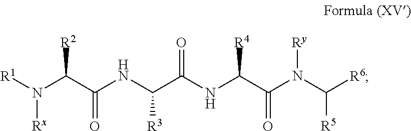

wherein:
R¹ is selected from:

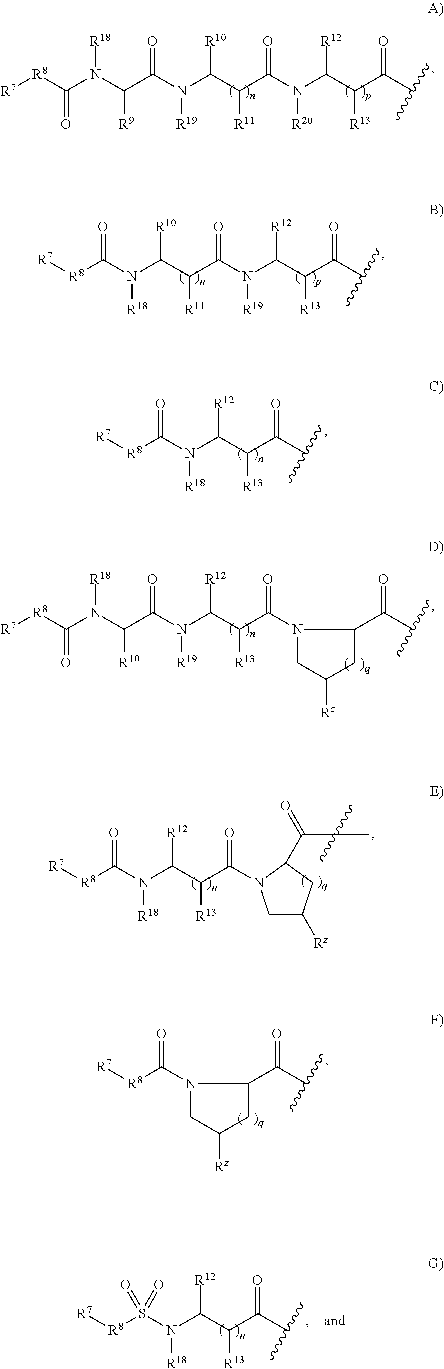

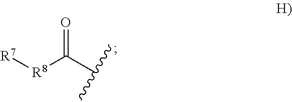

R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

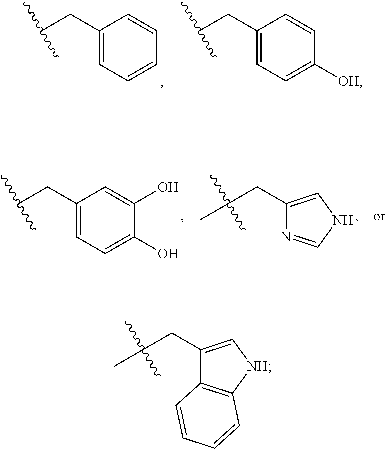

R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —C(═O)H, —CH₂C(═O)H, —C(═O)NHCH₂C(═O)H, —C(═O)C(═O)N(R¹⁴)₂, —C(═O)C(═O)OH, —C(═O)R²⁷, —B(OR²³)(OR²⁴), or

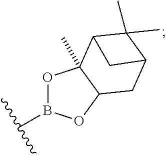 [SMILES: *B1OC2CC3CC(C3(C)C)[C@]2(C)O1]

or R⁵ and R⁶ together with the carbon atom form

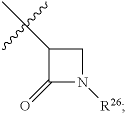

R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is

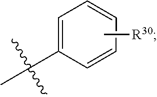

R⁸ is a bond, —O—, or —N(R¹⁷)—, optionally substituted aryl, or optionally substituted heteroaryl;
R⁹ is —CH₂OH, —CH₂CH(CH₃)₂,

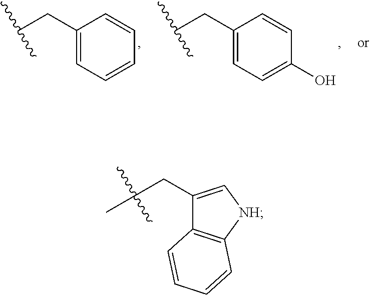

R¹⁴, R¹⁵, and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ and R²⁴ are each independently H, or C₁-C₄alkyl; or R²³ and R²⁴ together with the boron atom form an optionally substituted 5- or 6-membered boron containing ring;
each R²⁵ is independently C₁-C₆alkyl;
R²⁶ is H, C₁-C₄alkyl, C₁-C₄alkoxy, —CH₂C(O)OR²⁵, or —OCH₂C(O)OR²⁵;
R²⁷ is C₁-C₆alkyl, C₁-C₆haloalkyl, optionally substituted heteroaryl, —C(O)OR³², —CF₂C(O)OH, or

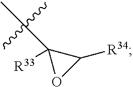

R³⁰ is

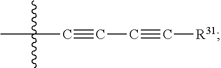

R³¹ is optionally substituted C₁-C₆alkyl;
R³² is optionally substituted C₁-C₆alkyl;
R³³ is H, C₁-C₄alkyl, or C₁-C₄alkoxy;
R³⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl,

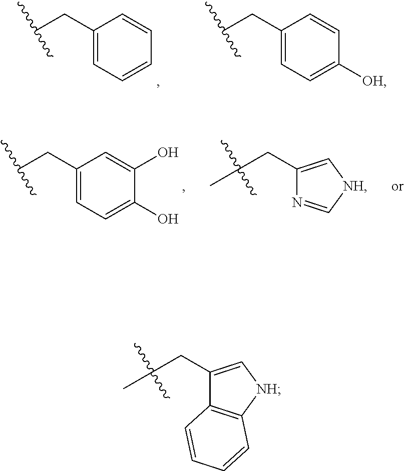 [SMILES: *Cc1ccccc1 ; *Cc1ccc(O)cc1 ; *Cc1ccc(O)c(O)c1 ; *Cc1c[nH]cn1 ; *Cc1c[nH]c2ccccc12]

n is 0 or 1;
p is 0 or 1; and
q is 0 or 1;
   or a pharmaceutically acceptable salt, solvate, or prodrug thereof

In another embodiment is a compound of Formula (XV) or Formula (XV′) wherein R¹ is

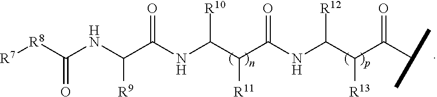

In a further embodiment, R⁸ is a bond. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

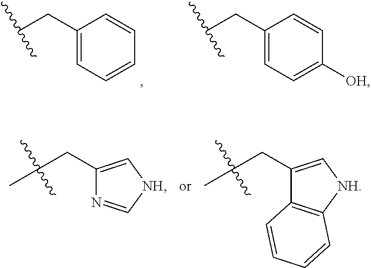 [SMILES: *Cc1ccccc1 ; *Cc1ccc(O)cc1 ; *Cc1c[nH]cn1 ; *Cc1c[nH]c2ccccc12]

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

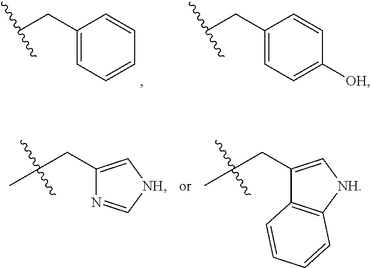 [SMILES: *Cc1ccccc1 ; *Cc1ccc(O)cc1 ; *Cc1c[nH]cn1 ; *Cc1c[nH]c2ccccc12]

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

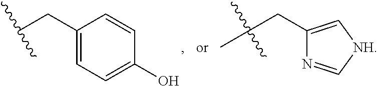 [SMILES: *Cc1ccc(O)cc1 ; *Cc1c[nH]cn1]

In a further embodiment of the aforementioned embodiments is a compound of Formula (XV) or Formula (XV′) wherein n is 0 and p is 0. In another embodiment, n is 0 and p is 1. In yet a further embodiment, n is 1 and p is 0.

In a further embodiment is a compound of Formula (XV′) having the structure of Formula (XVa):

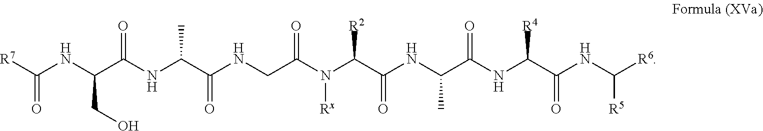

In another embodiment is a compound of Formula (XVa) wherein R² is —CH(OH)(CH₃), —CH₂CH₂C(O)OH, or —(CH₂)₄NH₂. In some embodiments, R² is —CH(OH)(CH₃). In some embodiments, R² is —CH₂CH₂C(O)OH. In some embodiments, R² is —(CH₂)₄NH₂. In a further embodiment is a compound of Formula (XVa) wherein R⁴ is CH₂CH(CH₃)₂ or —CH₂C(O)NH₂. In some embodiments, R⁴ is CH₂CH(CH₃)₂. In some embodiments, R⁴ is —CH₂C(O)NH₂. In yet a further embodiment is a compound of Formula (XVa) wherein R⁵ is H or —CH₃. In some embodiments, R⁴ is H. In some embodiments, R⁴ is —CH₃.

In another embodiment is a compound of Formula (XV) or Formula (XV′) wherein R¹ is

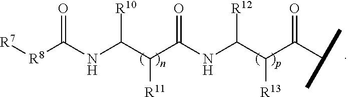

In a further embodiment, R⁸ is a bond. In another embodiment, R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

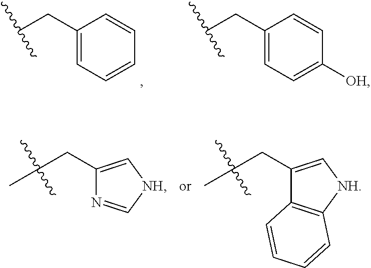 [SMILES: *Cc1ccccc1 ; *Cc1ccc(O)cc1 ; *Cc1c[nH]cn1 ; *Cc1c[nH]c2ccccc12]

In a further embodiment, R², R⁴, R¹⁰, R¹¹, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

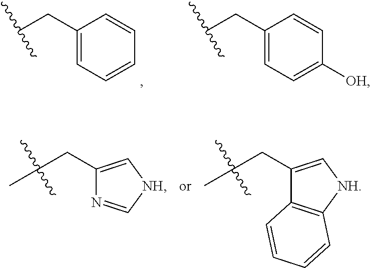 [SMILES: *Cc1ccccc1 ; *Cc1ccc(O)cc1 ; *Cc1c[nH]cn1 ; *Cc1c[nH]c2ccccc12]

In yet a further embodiment, R², R⁴, R¹⁰, R¹¹, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂, or

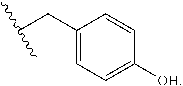 [SMILES: *Cc1ccc(O)cc1]

In a further embodiment of the aforementioned embodiments is a compound of Formula (XV) or Formula (XV′) wherein n is 0 and p is 0. In another embodiment, n is 0 and p is 1. In yet a further embodiment, n is 1 and p is 0.

In a further embodiment is a compound of Formula (XV′) having the structure of Formula (XVb):

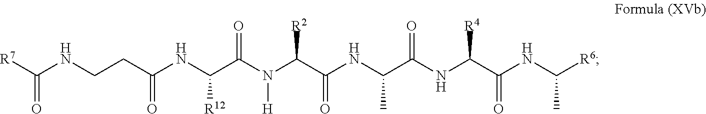

wherein R², R⁴, and R¹², are each independently —CH₂CH(CH₃)₂, —(CH₂)₃NH₂, or —(CH₂)₄NH₂.

In another embodiment is a compound of Formula (XVb) wherein R², R⁴, and R¹² are each —(CH₂)₄NH₂. In another embodiment is a compound of Formula (XVb) wherein R², R⁴, and R¹² are each —(CH₂)₃NH₂. In another embodiment is a compound of Formula (XVb) wherein R⁴ is —CH₂CH(CH₃)₂, R² is —(CH₂)₃NH₂, and R¹² is —(CH₂)₄NH₂. In another embodiment is a compound of Formula (XVb) wherein R⁴ is —CH₂CH(CH₃)₂, R² is —(CH₂)₄NH₂, and R¹² is —(CH₂)₄NH₂.

In a further embodiment is a compound of Formula (XV′) having the structure of Formula (XVbb):

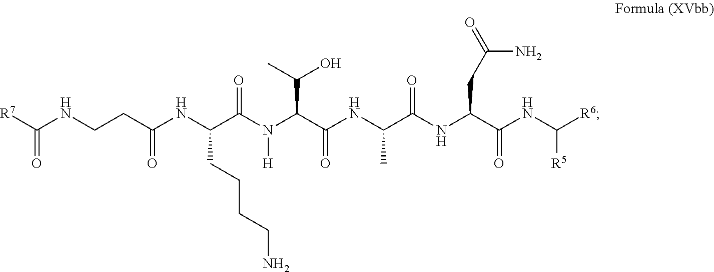

wherein R⁵ is —H, or —CH₃.

In a further embodiment is a compound of Formula (XV′) having the structure of Formula (XVbbb):

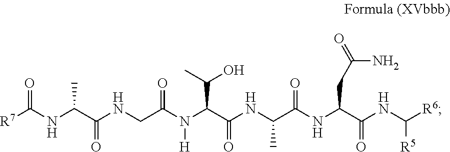

wherein R⁵ is —H, or —CH₃.

In another embodiment is a compound of Formula (XV) or Formula (XV′) wherein R¹ is

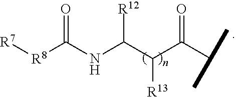

In a further embodiment, R⁸ is a bond. In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

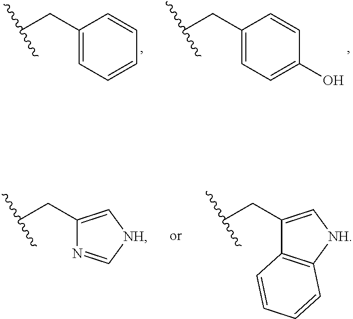 [SMILES: *Cc1ccccc1 ; *Cc1ccc(O)cc1 ; *Cc1c[nH]cn1 ; "or" ; *Cc1c[nH]c2ccccc12]

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

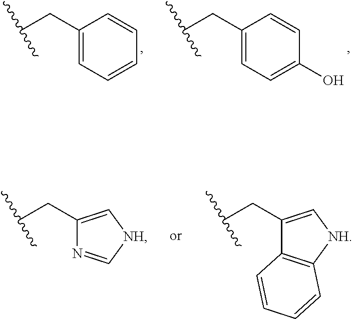 [SMILES: *Cc1ccccc1 ; *Cc1ccc(O)cc1 ; *Cc1c[nH]cn1 ; "or" ; *Cc1c[nH]c2ccccc12]

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

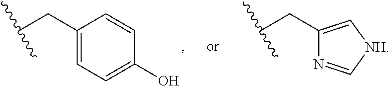 [SMILES: *Cc1ccc(O)cc1 ; *Cc1c[nH]cn1]

In a further embodiment of the aforementioned embodiments is a compound of Formula (XV) or Formula (XV′) wherein n is 0. In yet a further embodiment, n is 1.

In a further embodiment is a compound of Formula (XV′) having the structure of Formula (XVc):

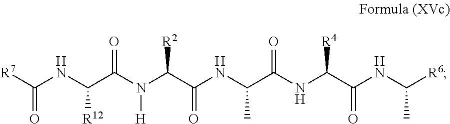

wherein R², R⁴, and R¹², are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (XVc) wherein R⁴ is —(CH₂)₄NH₂, R² is —CH(OH)(CH₃), and R¹² is —(CH₂)₂NH₂. In another embodiment is a compound of Formula (XVc) wherein R⁴ is —(CH₂)₄NH₂, R² is —CH(OH)(CH₃), and R¹² is —CH₂NH₂. In another embodiment is a compound of Formula (XVc) wherein R⁴ is —CH₂C(O)NH₂, R² is —CH(OH)(CH₃), and R¹² is —(CH₂)₄NH₂. In another embodiment is a compound of Formula (XVc) wherein R⁴ is —(CH₂)₄NH₂, R² is —(CH₂)₄NH₂, and R¹² is —CH₂NH₂. In another embodiment is a compound of Formula (XVc) wherein R⁴ is —CH₂C(O)NH₂, R² is —(CH₂)₄NH₂, and R¹² is —CH₂NH₂. In another embodiment is a compound of Formula (XVc) wherein R⁴ is —CH₂CH(CH₃)₂, R² is —(CH₂)₂NH₂, and R¹² is —(CH₂)₂NH₂.

In a further embodiment is a compound of Formula (XV′) having the structure of Formula (XVcc):

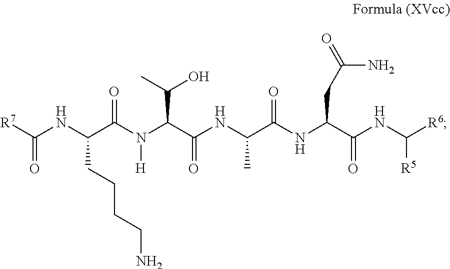

wherein R⁵ is —H, or —CH₃.

In another embodiment is a compound of Formula (XV) or Formula (XV′) wherein R¹ is

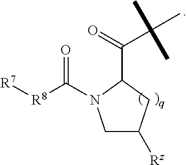

In a further embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

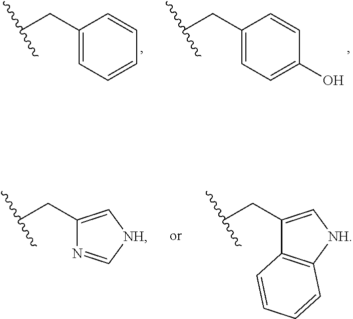 [SMILES: *Cc1ccccc1 ; *Cc1ccc(O)cc1 ; *Cc1c[nH]cn1 ; "or" ; *Cc1c[nH]c2ccccc12]

In a further embodiment, q is 1 and R⁸ is a bond.

In a further embodiment is a compound of Formula (XV′) having the structure of Formula (XVd):

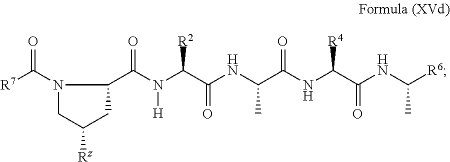

wherein R^{z} is NH₂; and R² and R⁴ are each independently —CH₂CH(CH₃)₂, —CH(OH)(CH₃), —CH₂C(O)NH₂, —CH₂CH₂C(O)NH₂, —CH₂NH₂, —(CH₂)₂NH₂, —(CH₂)₃NH₂, or —(CH₂)₄NH₂.

In another embodiment is a compound of Formula (XVd) wherein R² is —CH(OH)(CH₃), and R⁴ is —CH₂C(O)NH₂. In another embodiment is a compound of Formula (XVd) wherein R² is —CH(OH)(CH₃), and R⁴ is —(CH₂)₂NH₂. In another embodiment is a compound of Formula (XVd) wherein R² is —CH(OH)(CH₃), and R⁴ is —(CH₂)₃NH₂. In another embodiment is a compound of Formula (XVd) wherein R² is —CH(OH)(CH₃), and R⁴ is —(CH₂)₄NH₂. In another embodiment is a compound of Formula (XVd) wherein R² is —(CH₂)₄NH₂ and R⁴ is —CH₂CH(CH₃)₂. In another embodiment is a compound of Formula (XVd) wherein R² is —(CH₂)₄NH₂ and R⁴ is —CH₂C(O)NH₂. In another embodiment is a compound of Formula (XVd) wherein R² is —(CH₂)₄NH₂ and R⁴ is —(CH₂)₄NH₂.

In another embodiment is a compound of Formula (XV) or Formula (XV′) wherein R¹ is

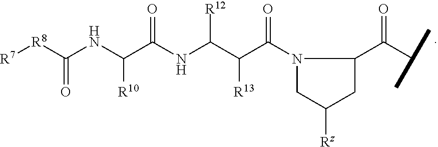

In a further embodiment, R⁸ is a bond. In another embodiment, R², R⁴, R¹⁰, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

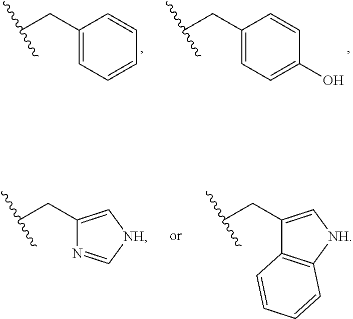 [SMILES: *Cc1ccccc1 ; *Cc1ccc(O)cc1 ; *Cc1c[nH]cn1 ; "or" ; *Cc1c[nH]c2ccccc12]

In a further embodiment, R², R⁴, R¹⁰, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

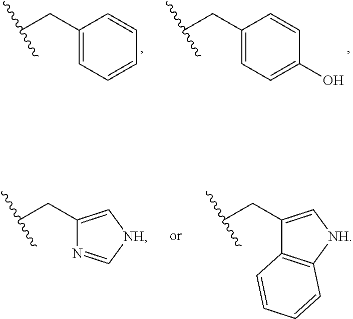 [SMILES: *Cc1ccccc1 ; *Cc1ccc(O)cc1 ; *Cc1c[nH]cn1 ; "or" ; *Cc1c[nH]c2ccccc12]

In yet a further embodiment, R², R⁴, R¹⁰, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

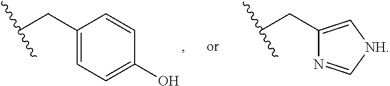 [SMILES: *Cc1ccc(O)cc1 ; *Cc1c[nH]cn1]

In a further embodiment of the aforementioned embodiments is a compound of Formula (XV) or Formula (XV′) wherein n is 0. In yet a further embodiment, n is 1.

In a further embodiment is a compound of Formula (XV′) having the structure of Formula (XVdd):

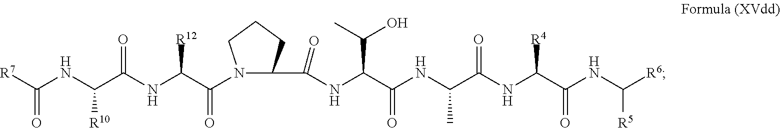

wherein R⁵ is —H, or —CH₃.

In another embodiment is a compound of Formula (XVdd) wherein R¹⁰ is —CH₂OH, and R¹² is —CH₃. In another embodiment is a compound of Formula (Idd) wherein R¹⁰ is —CH₂CH(CH₃)₂, and R¹² is —CH(OH)(CH₃). In another embodiment of the aforementioned compounds of Formula (XVdd) is a compound wherein R⁴ is —CH₂C(O)NH₂. In yet another embodiment of the aforementioned compounds of Formula (XVdd) is a compound wherein R⁴ is

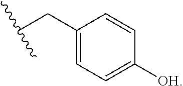 [SMILES: *Cc1ccc(O)cc1]

In another embodiment is a compound of Formula (XV) or Formula (XV′) wherein R¹ is

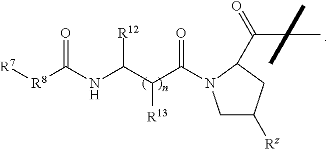

In a further embodiment, R⁸ is a bond. In another embodiment, R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

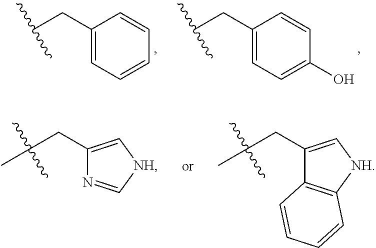

In a further embodiment, R², R⁴, R¹², and R¹³ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

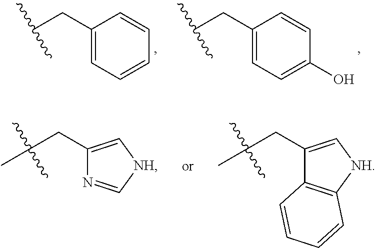

In yet a further embodiment, R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂NH₂, —CH₂CH₂C(O)NH₂, —(CH₂)₂NH₂, —(CH₂)₃NH₂, —(CH₂)₄NH₂,

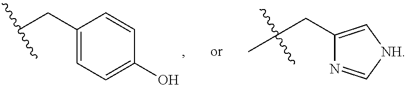 [SMILES: *Cc1ccc(O)cc1 ; *Cc1c[nH]cn1]

In a further embodiment of the aforementioned embodiments is a compound of Formula (XV) or Formula (XV′) wherein n is 0. In yet a further embodiment, n is 1.

In another embodiment is a compound of Formula (XV) or Formula (XV′) wherein R¹ is

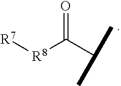

In a further embodiment, R⁸ is a bond. In another embodiment, R² and R⁴ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

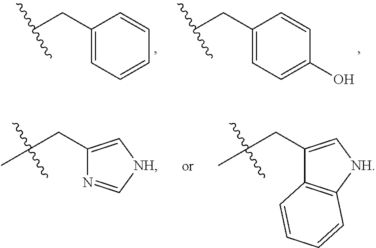

In a further embodiment, R² and R⁴ are each independently —H, —CH₃, —CH(CH₃)₂, —CH(CH₃)(CH₂CH₃), —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

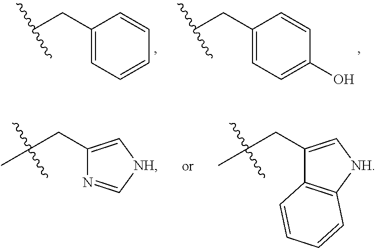

In yet a further embodiment, R² and R⁴ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —(CH₂)₂NH₂, —(CH₂)₃NH₂, —(CH₂)₄NH₂,

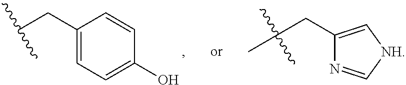 [SMILES: *Cc1ccc(O)cc1 ; *Cc1c[nH]cn1]

In another embodiment is a compound of Formula (XV) or Formula (XV′) wherein R^{x} and R² together with the nitrogen atom form an optionally substituted nitrogen containing ring. In a further embodiment is a compound of Formula (XV′) having the structure of Formula (XVe):

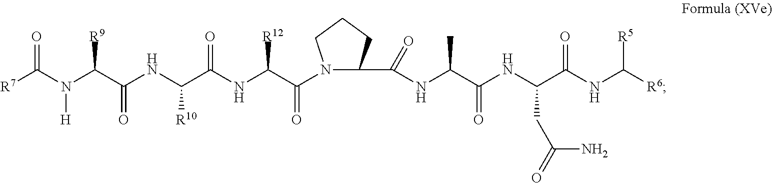

wherein R⁵ is —H, or —CH₃.

In another embodiment is a compound of Formula (XVe) wherein R¹⁰ and R¹² are each independently —H, —CH₃, —CH₂CH(CH₃)₂, —CH₂OH, or —CH(OH)(CH₃).

In another embodiment of any of the aforementioned embodiments of Formula (XV) or Formula (XV′) is a compound wherein R⁶ is

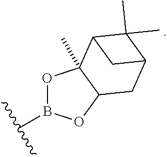 [SMILES: *B1OC2CC3CC(C3(C)C)[C@]2(C)O1]

In another embodiment of any of the aforementioned embodiments of Formula (XV) or Formula (XV′) is a compound wherein R⁶ is B(OH)₂. In another embodiment of any of the aforementioned embodiments of Formula (XV) or Formula (XV′) is a compound wherein R⁶ is —C(═O)H. In another embodiment of any of the aforementioned embodiments of Formula (XV) or Formula (XV′) is a compound wherein R⁶ is —C(═O)C(═O)OH. In another embodiment of any of the aforementioned embodiments of Formula (XV) or Formula (XV′) is a compound wherein R⁶ is —C(═O)R²⁷. In another embodiment of any of the aforementioned embodiments of Formula (XV) or Formula (XV′) is a compound wherein R⁶ is —C(═O)R²⁷ and R²⁷ is —C(O)OR³². In another embodiment of any of the aforementioned embodiments of Formula (XV) or Formula (XV′) is a compound wherein R⁶ is —C(═O)R²⁷ and R²⁷ is

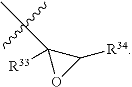

In another embodiment of any of the aforementioned embodiments of Formula (XV) or Formula (XV′) is a compound wherein R⁸ is a bond and R⁷ is

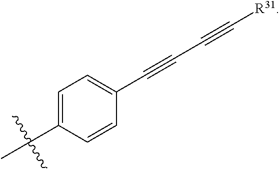

In another embodiment is a compound selected from:

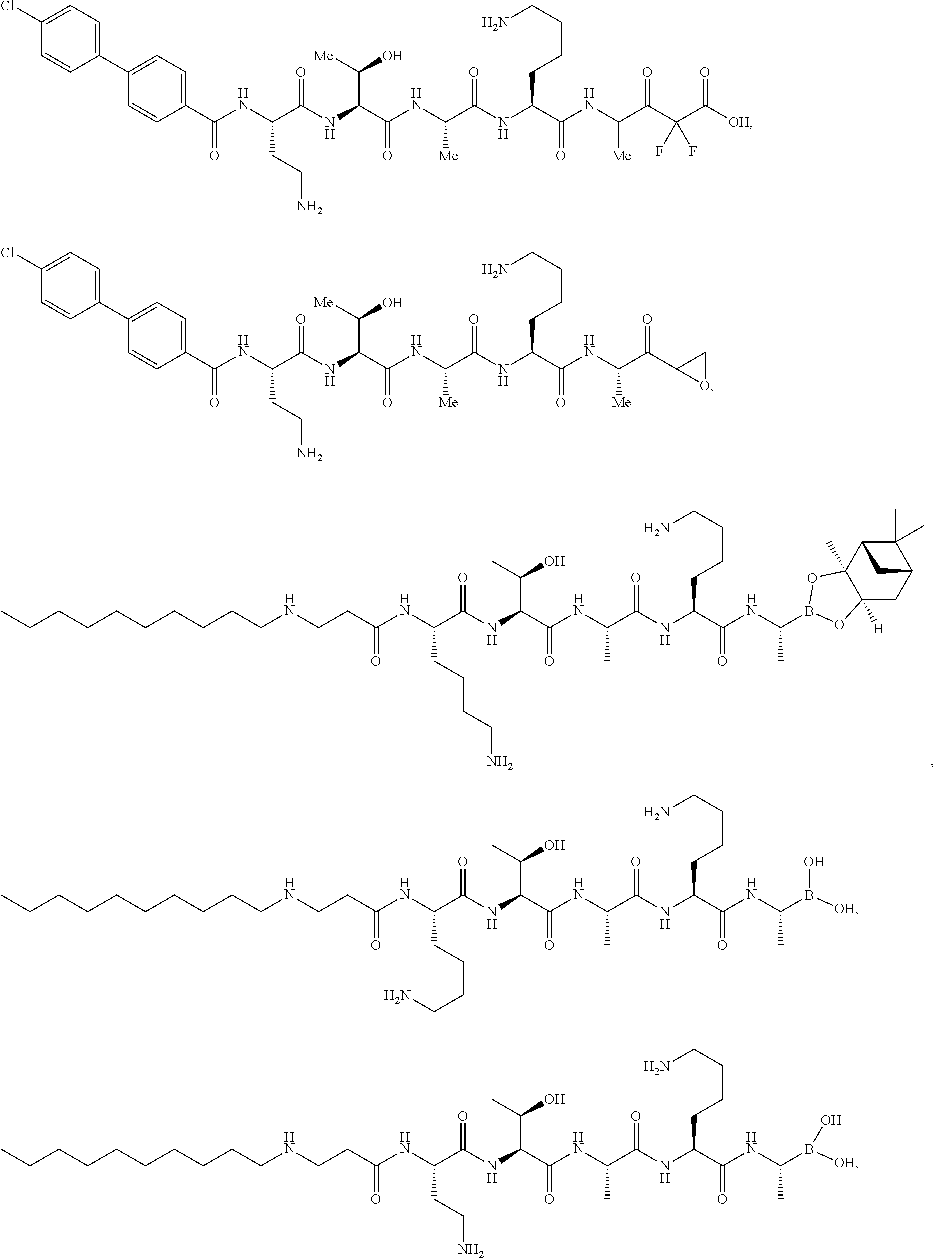

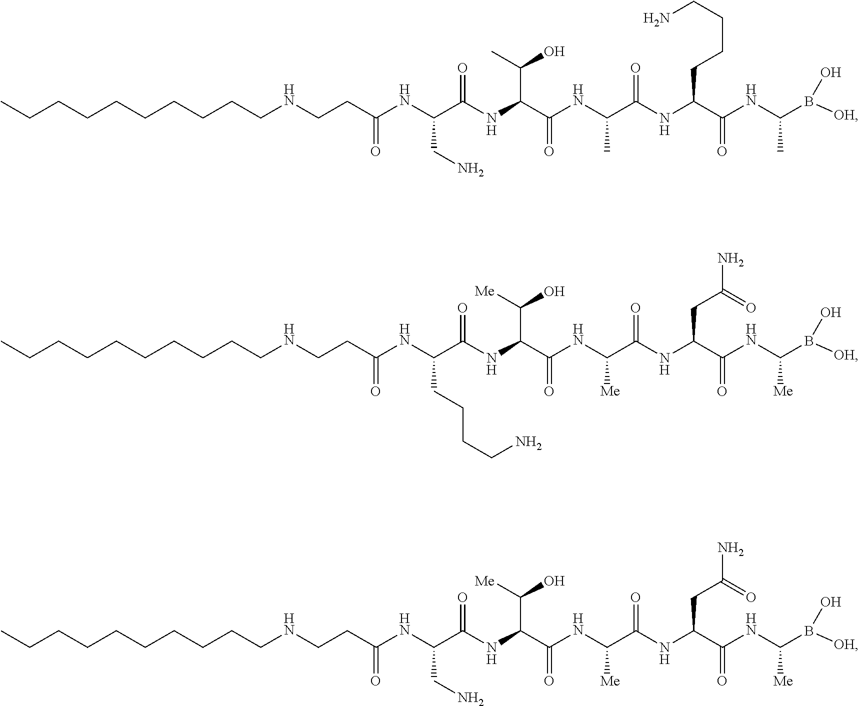

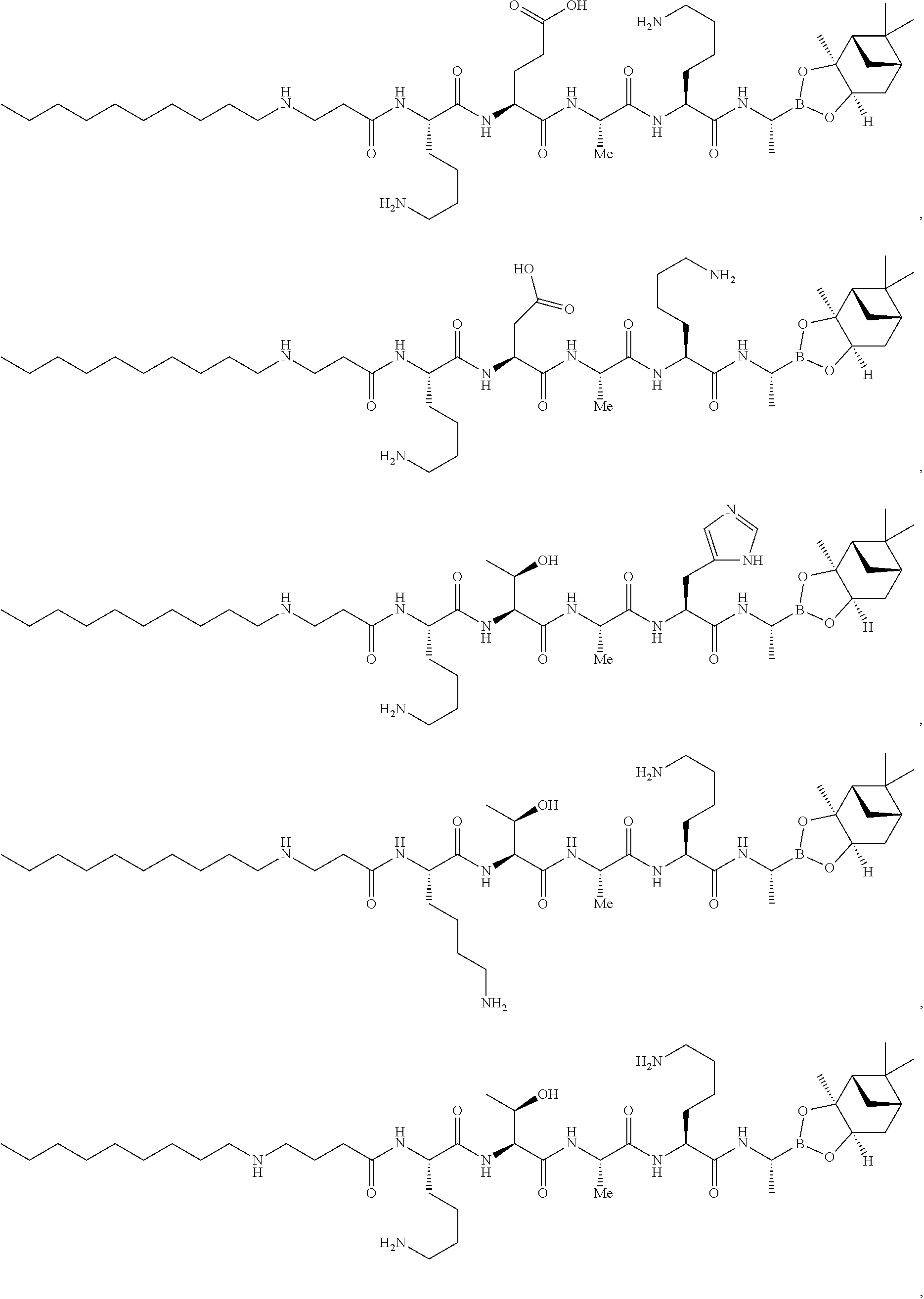

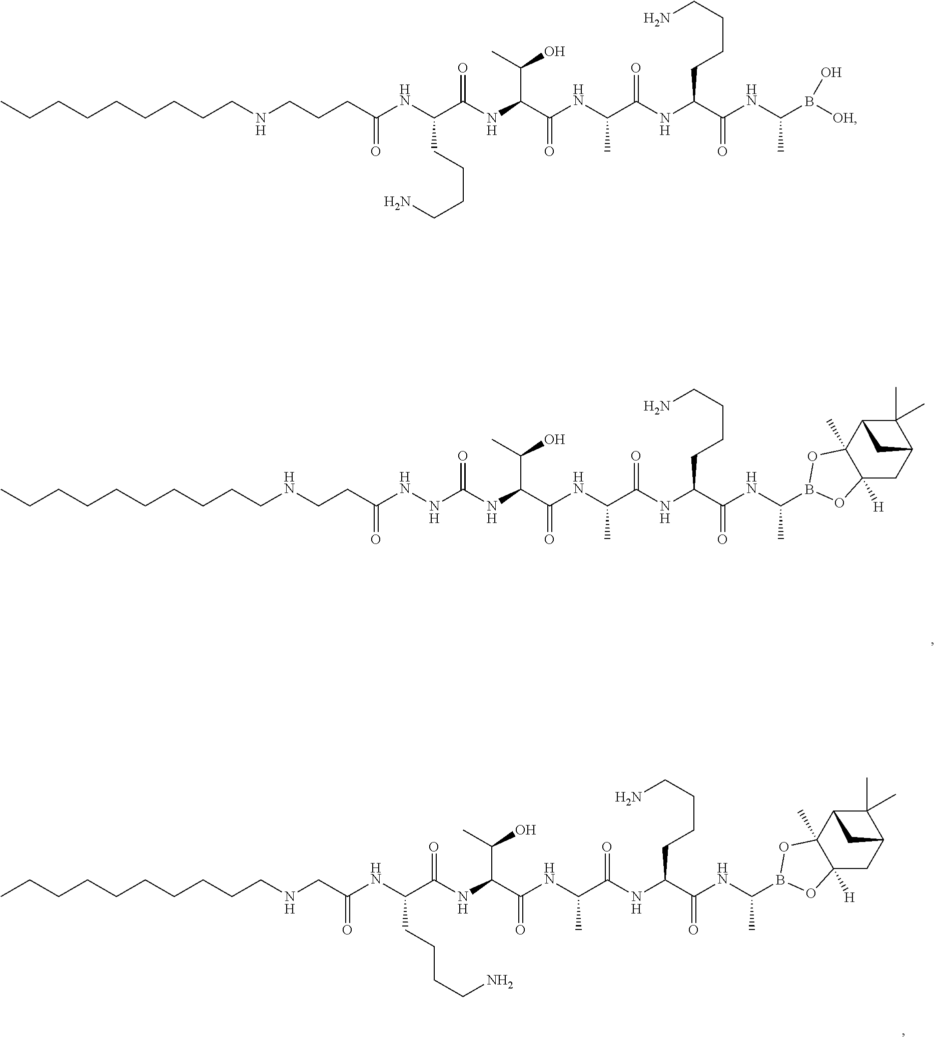

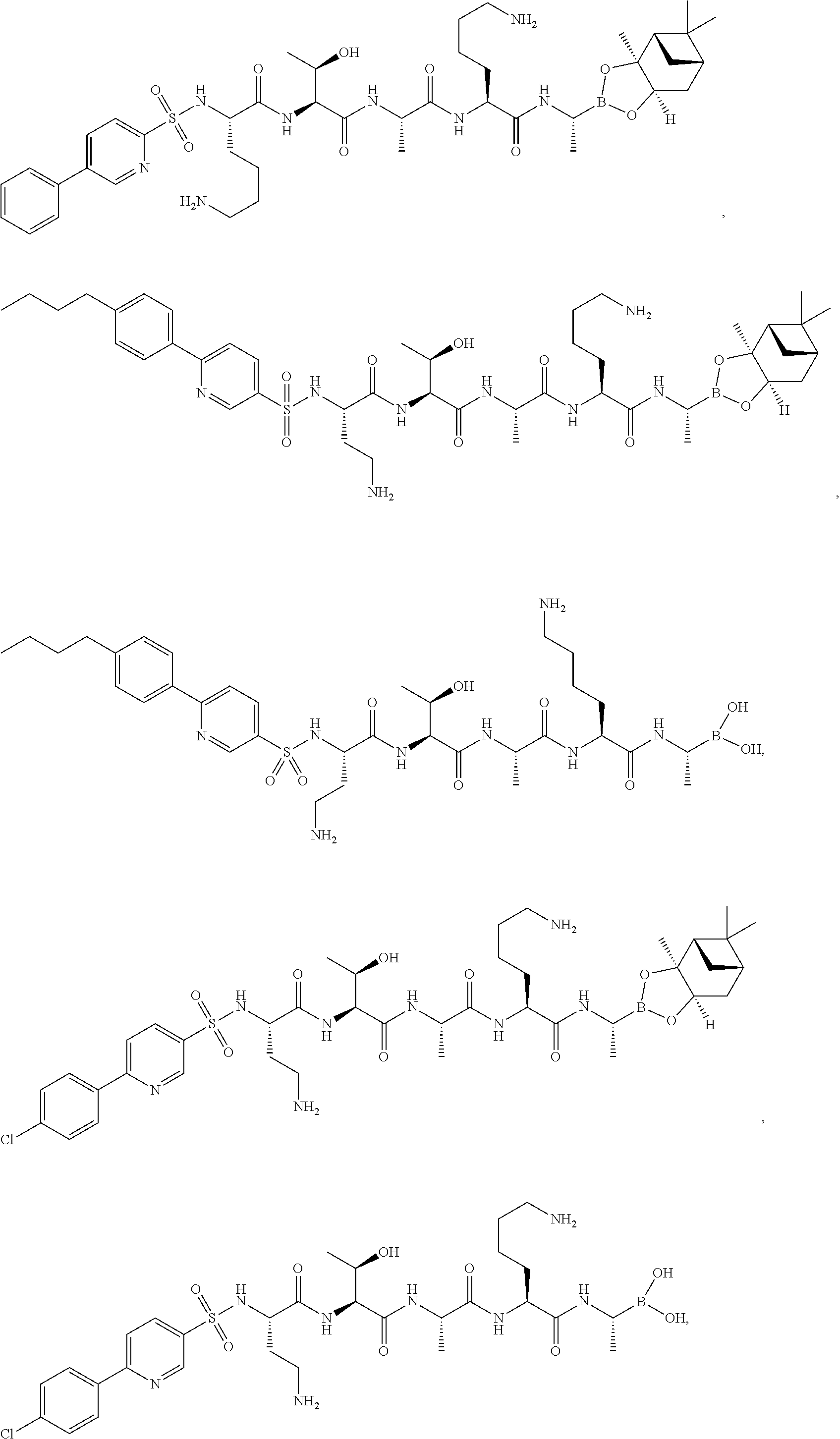

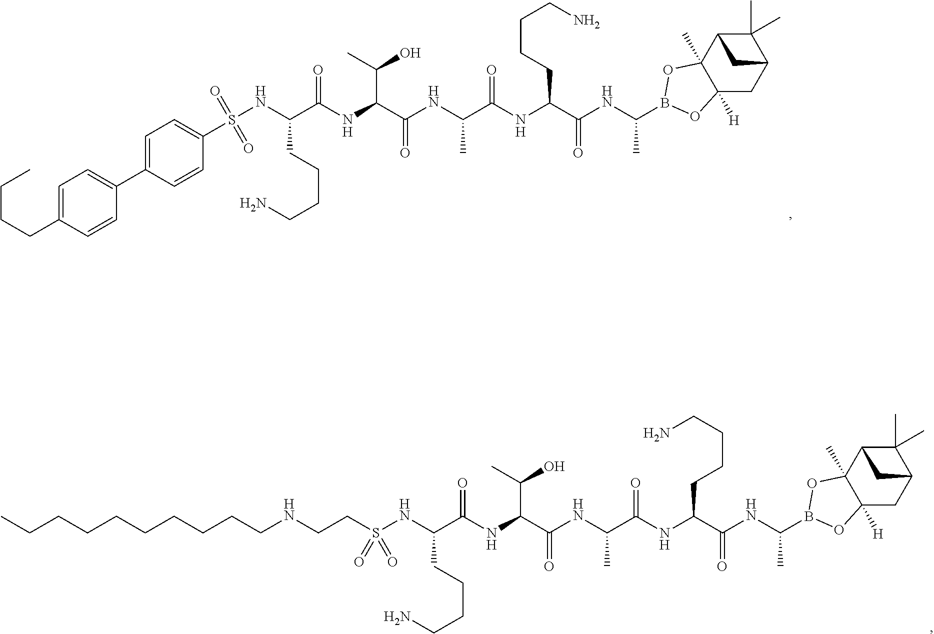

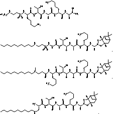

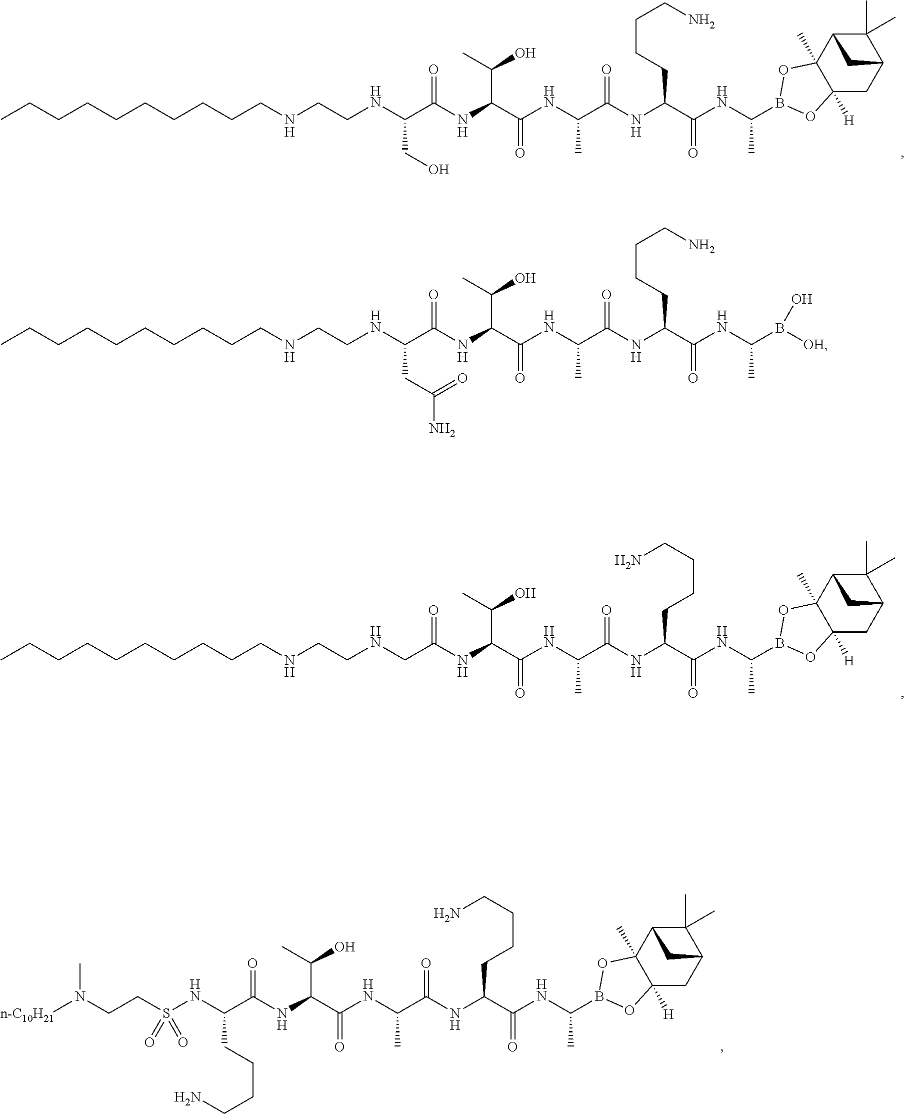

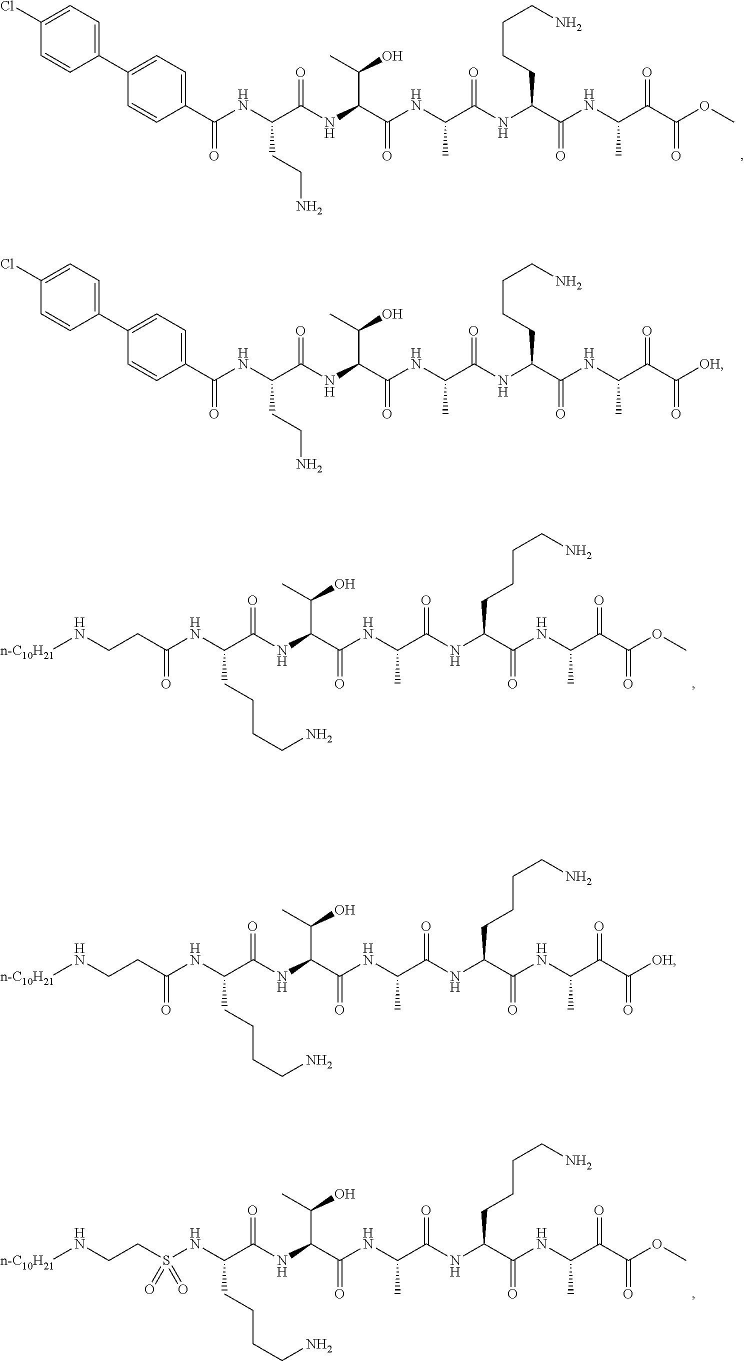

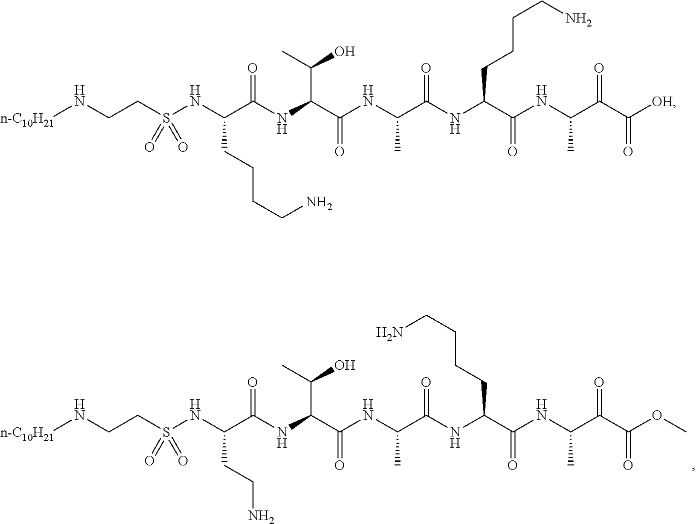

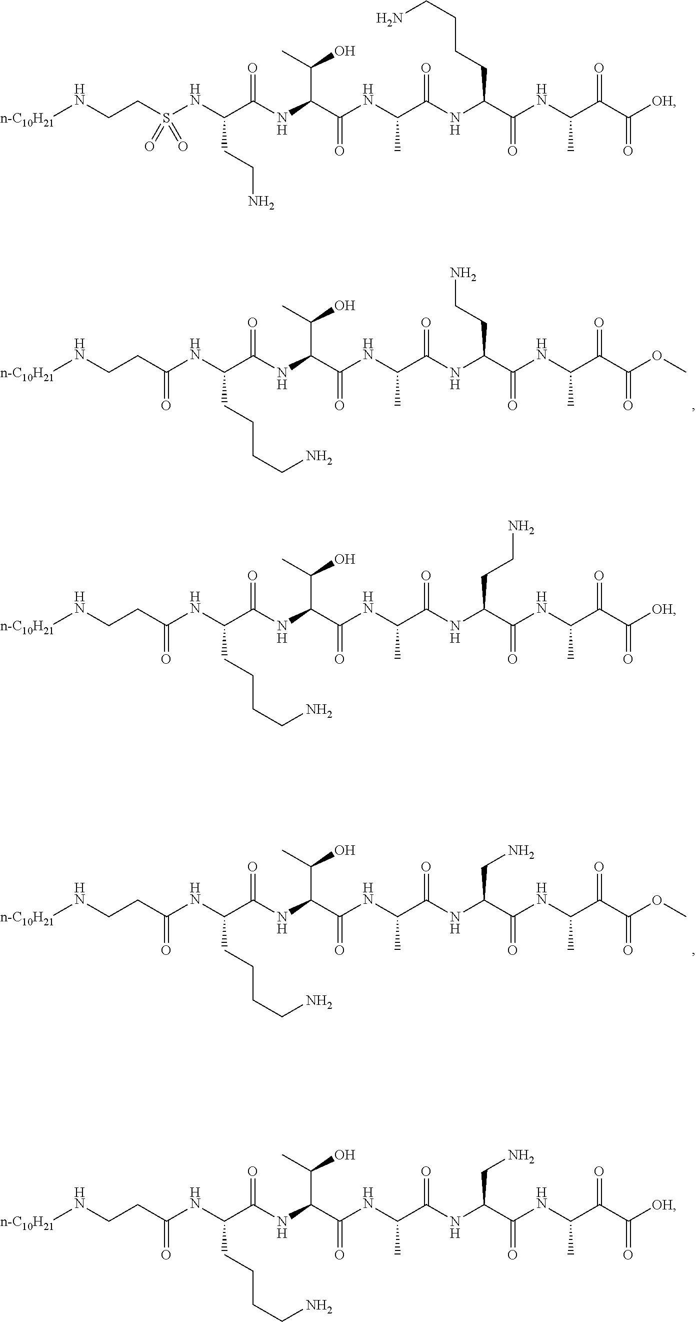

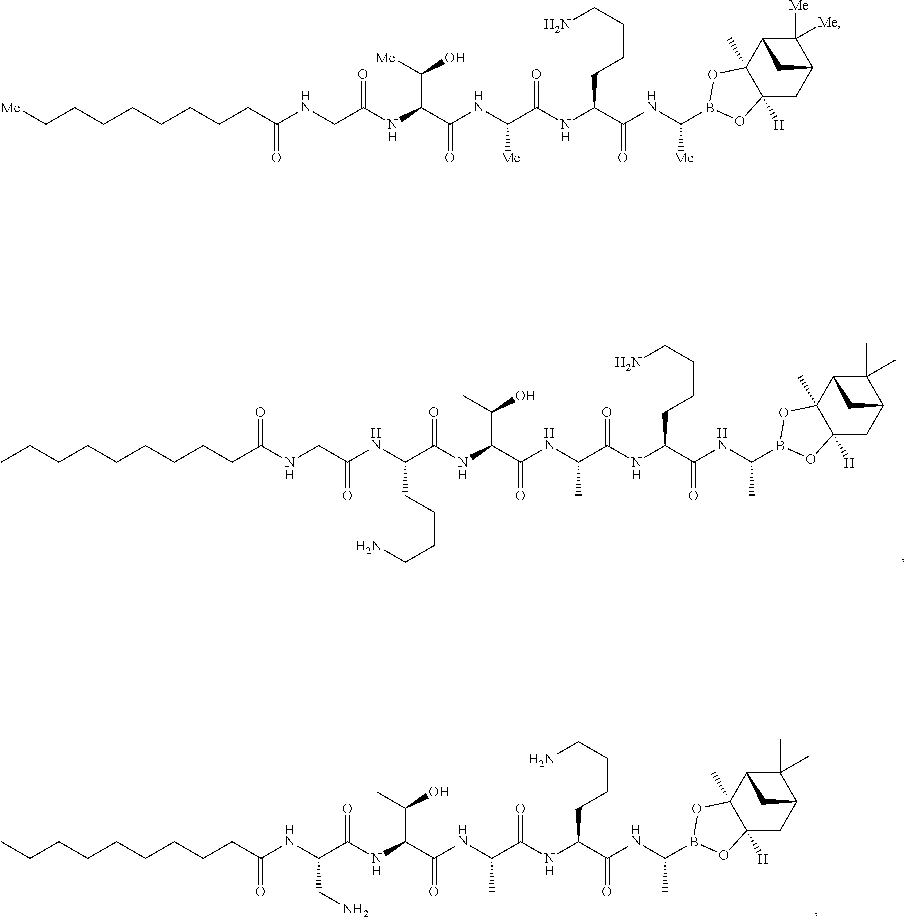

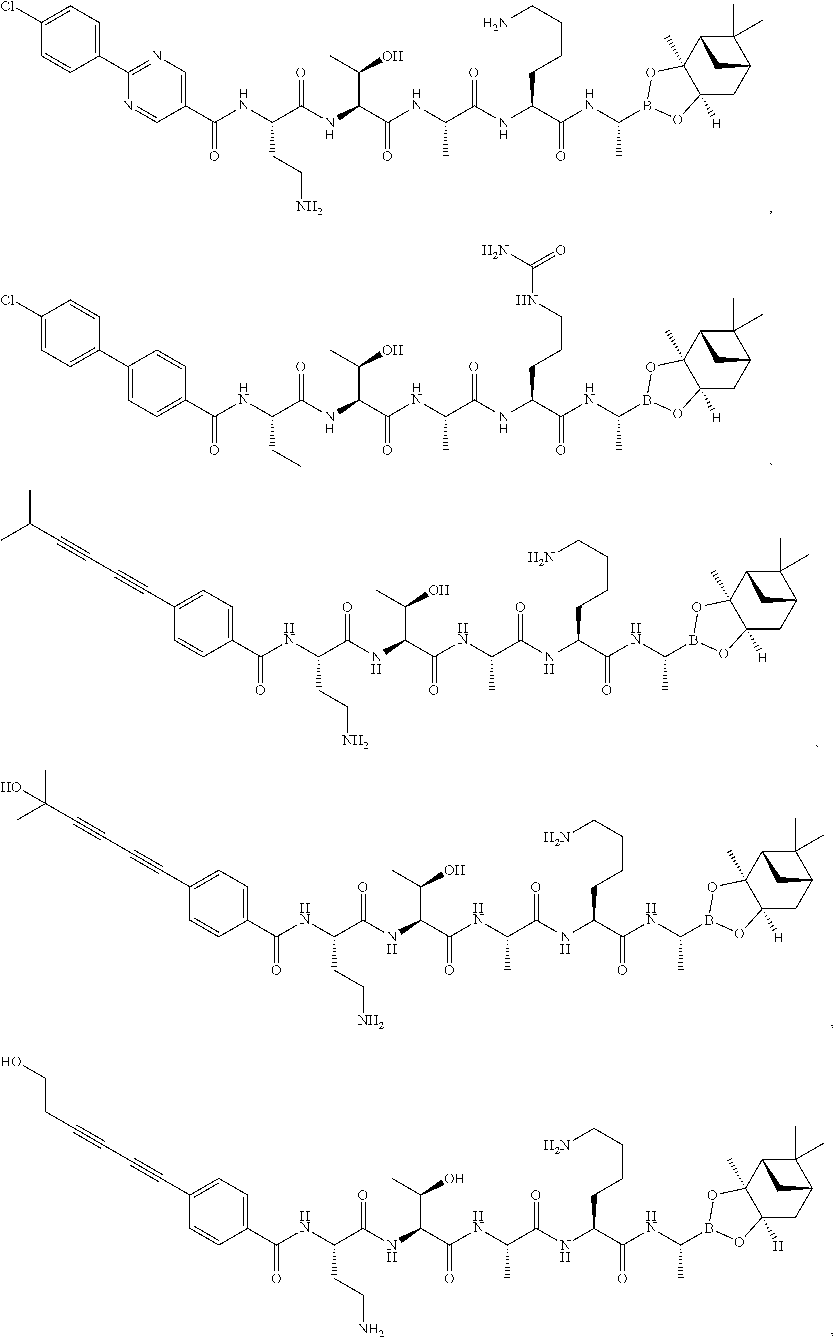

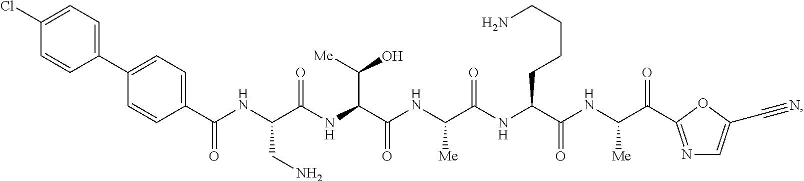 [SMILES: C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)c1ncc(C#N)o1]

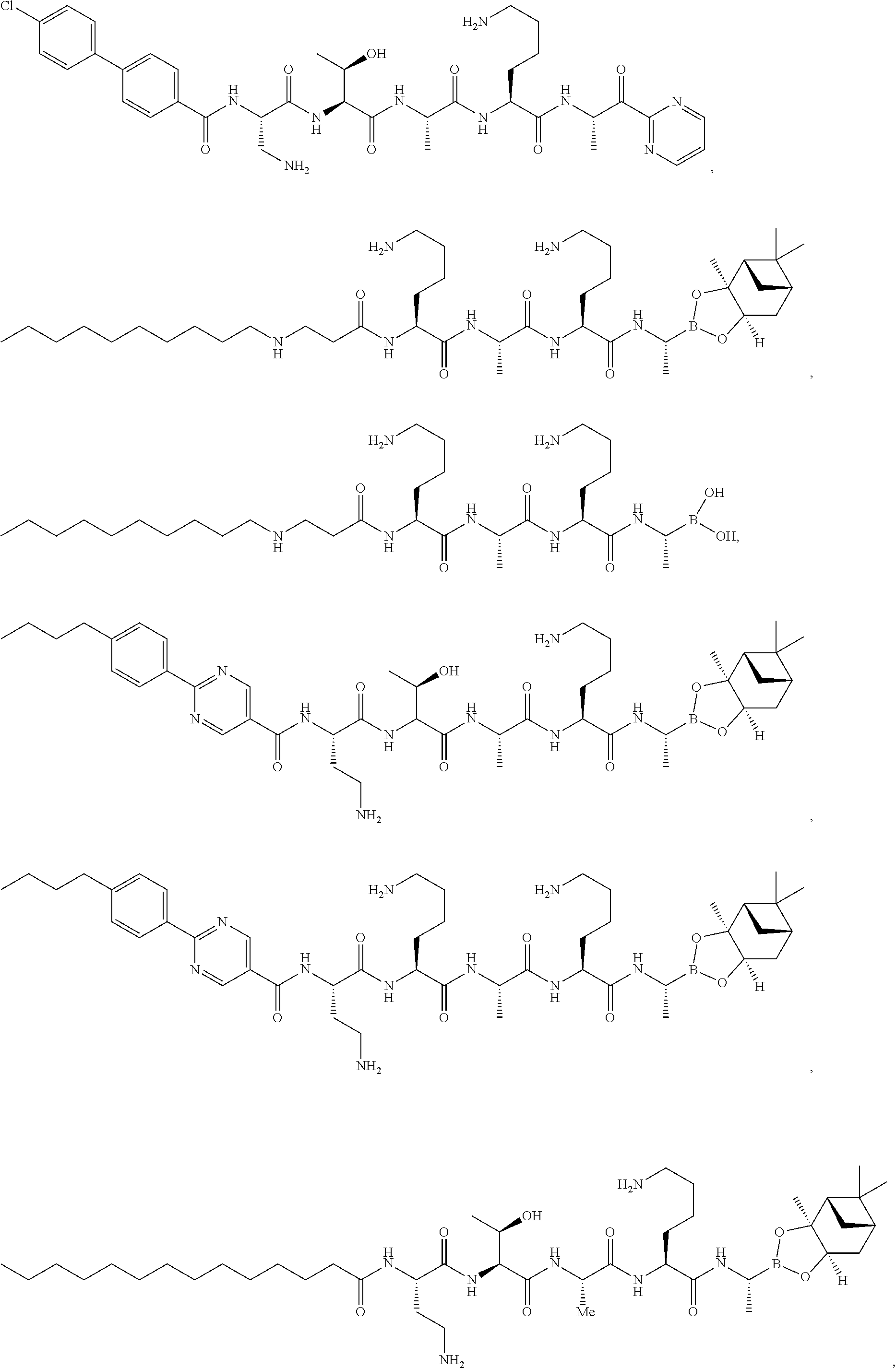

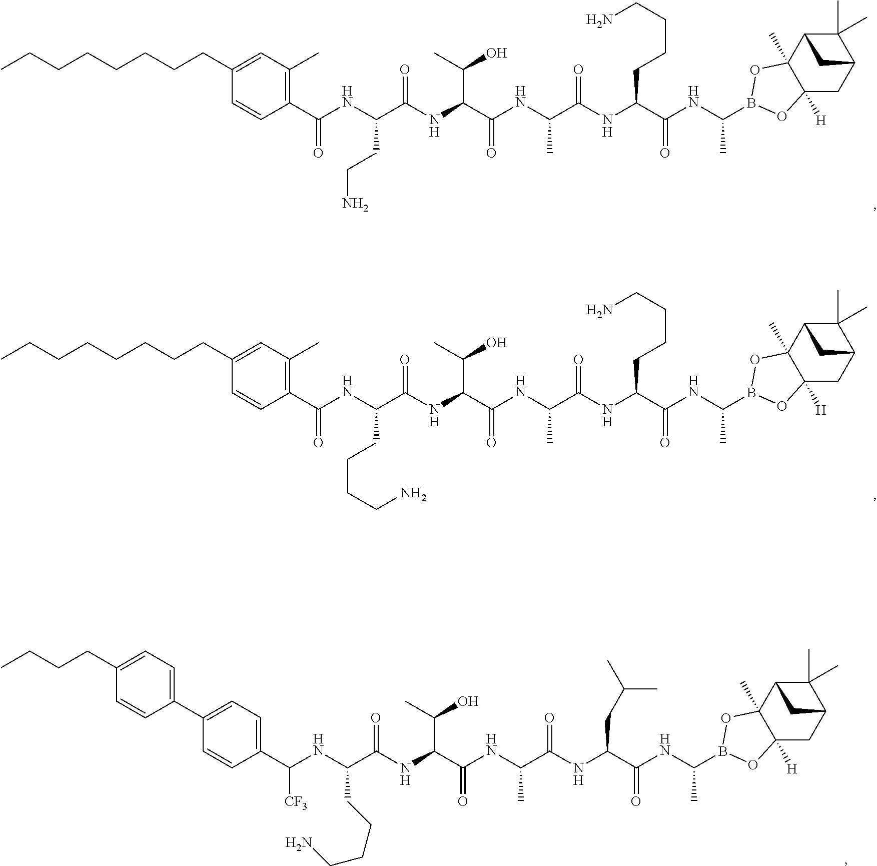

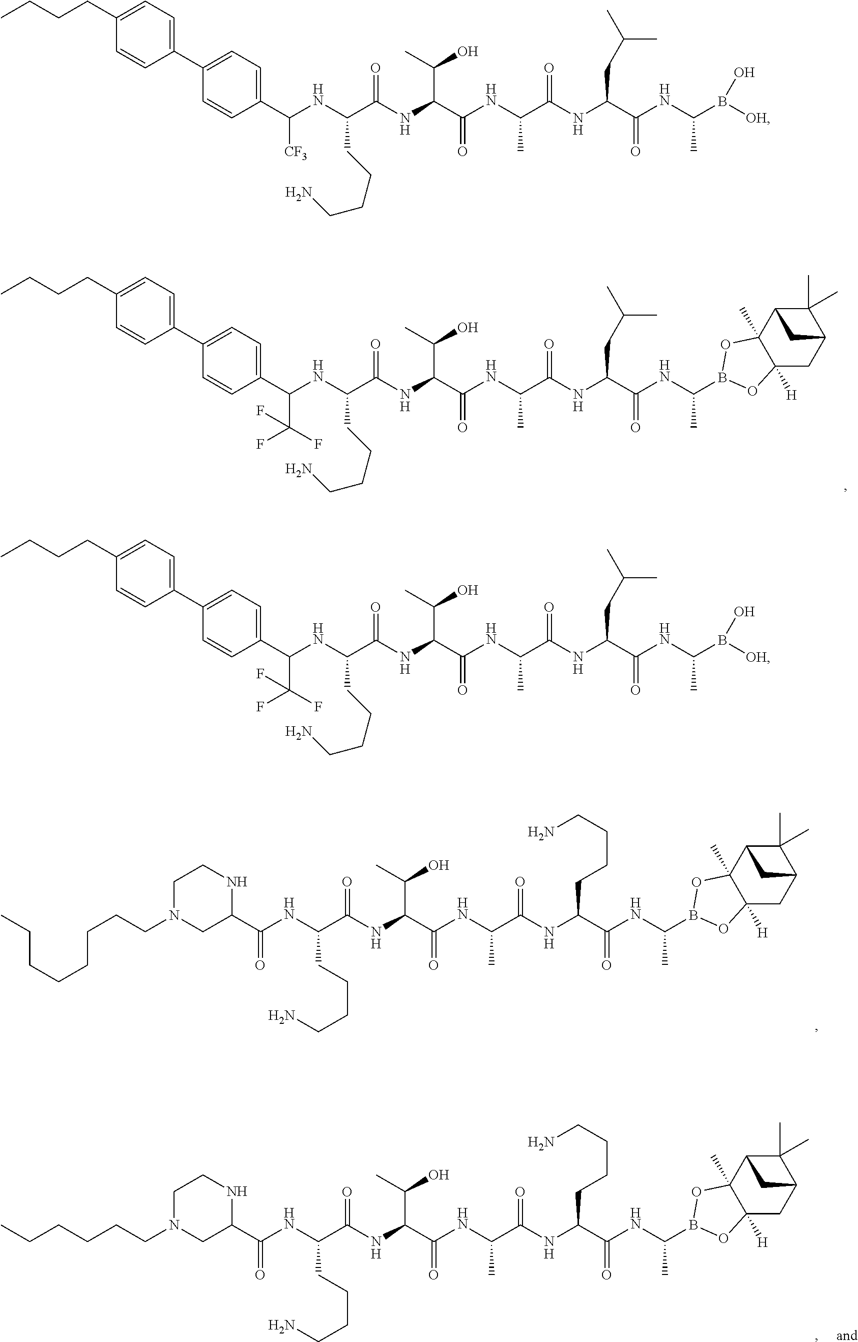

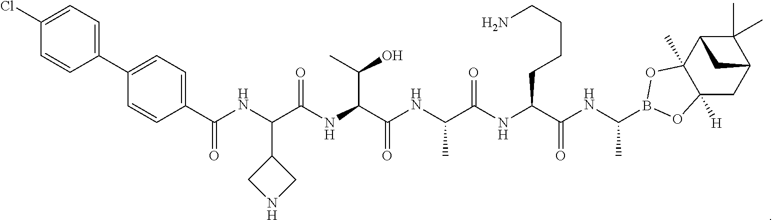 [SMILES: C[C@H](NC(=O)[C@H](CCCCN)NC(=O)[C@H](C)NC(=O)[C@@H](NC(=O)C(NC(=O)c1ccc(-c2ccc(Cl)cc2)cc1)C1CNC1)[C@@H](C)O)B1O[C@@H]2C[C@@H]3C[C@@H](C3(C)C)[C@]2(C)O1 ; "."]

In another aspect are hydrates, or metabolites comprising any of the aforementioned compounds.

In another aspect are pharmaceutical compositions comprising any of the aforementioned compounds together with a pharmaceutically acceptable excipient.

In another aspect described herein is the use of a compound described herein in the manufacture of a medicament for treatment of a bacterial infection in a patient.

In another aspect are methods of treating a mammal in need of such treatment comprising administering to the mammal an antibacterial effective amount of any of the aforementioned compounds at a frequency and for a duration sufficient to provide a beneficial effect to the mammal. In a further embodiment, the causative bacteria species of the bacteria infection is an infection involving Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus. In another embodiment the bacterial infection is an infection involving a gram negative bacteria. In a further embodiment, the bacterial infection is an infection involving a gram positive bacteria. In another embodiment, the mammal has a bacteria-related infection that is resistant to treatment with arylomycin A2.

In another aspect are methods of treating a mammal in need of such treatment comprising administering to the mammal arylomycin A and/or arylomycin B and/or any of the aforementioned compounds, wherein the infection involves a bacterial species that expresses a signal peptidase without a proline residue within 10 amino acids N-terminal to the signal peptidase catalytic serine. In a further embodiment, the bacterial species encodes or expresses an SPase enzyme without a proline residue 5 to 7 amino acids N-terminal to the SPase catalytic serine. In another embodiment, the bacteria infection is an infection involving Corynebacterium diphtheriae, Corynebacterium glutamicum, Campylobacter jejuni, Chlamydia trachomatis, Chlamydophila pneumoniae, Francisella tularensis, Helicobacter pylori, Lactococcus lactis subsp. cremoris, Lactococcus lactis subsp. lactis, Propionibacterium acnes, Rhodococcus equi, Staphylococcus carnosus, Staphylococcus cohnii, Staphylococcus haemolyticus, Staphylococcus hominis, Staphylococcus hominis subsp. hominis, Staphylococcus hominis subsp. novobiosepticus, Staphylococcus lugdunensis, Streptococcus agalactiae, Streptococcus dysgalactiae, Streptococcus mitis, Streptococcus oralis, Streptococcus pyogenes, and/or Streptococcus pnemoniae. In another embodiment the bacterial infection is an infection involving a gram negative bacteria. In another embodiment, administering comprises a topical administration.

In another aspect are methods of treating a mammal in need of such treatment comprising administering to the mammal any one or any combination of the aforementioned compounds, wherein the infection involves a bacterial species that expresses a signal peptidase without a proline residue within 10 amino acids N-terminal to the signal peptidase catalytic serine. In a further embodiment, the bacterial species encodes or expresses an SPase enzyme without a proline residue 5 to 7 amino acids N-terminal to the SPase catalytic serine. In another embodiment, the bacteria infection is an infection involving Staphylococcus capitis, Staphylococcus caprae and/or Yersinia pestis.

In a further embodiment are methods of treating a mammal in need of such treatment comprising administering to the mammal a second therapeutic agent to any of the aforementioned methods of treatment. In another embodiment, the second therapeutic agent is a non-arylomycin antibiotic. In another embodiment, the non-arylomycin antibiotic is an aminoglycoside antibiotic, fluoroquinolone antibiotic, penicillin antibiotic, cephalosporin antibiotic, macrolide antibiotic, glycopeptide antibiotic, rifampicin, chloramphenicol, fluoramphenicol, colistin, mupirocin, bacitracin, daptomycin, or linezolid.

In one embodiment, is a compound described herein which displays antibiotic activity useful in the treatment of bacterial infections, such as by way of example only, various strains of S. aureus, S. pneumoniae, E. faecalis, E. faecium, B. subtilis and E. coli including species that are resistant to many known antibiotics such as methicillin-resistant S. aureus (MRSA), vancomycin-resistant Enterococcus sp. (VRE), multidrug-resistant E. faecium, macrolide-resistant S. aureus and S. epidermidis, and linezolide-resistant S. aureus and E. faecium.

### Methicillin-Resistant Staphylococcus aureus

Staphylococcus aureus (S. aureus), a spherical bacterium, is the most common cause of staph infections. S. aureus has been known to cause a range of illnesses from minor skin infections, such as pimples, impetigo, boils, cellulitis folliculitis, furuncles, carbuncles, scalded skin syndrome, abscesses, to life-threatening diseases such as pneumonia, meningitis, osteomyelitis endocarditis, toxic shock syndrome, and septicemia. Further, S. aureus is one of the most common causes of nosocomial infections, often causing postsurgical wound infections.

Methicillin was introduced in the late 1950s to treat infections caused by penicillin-resistant S. aureus. It has been reported previously that S. aureus isolates had acquired resistance to methicillin (methicillin-resistant S. aureus, MRSA). The methicillin resistance gene (mecA) encodes a methicillin-resistant penicillin-binding protein that is not present in susceptible strains. mecA is carried on a mobile genetic element, the staphylococcal cassette chromosome mec (SCCmec), of which four forms have been described that differ in size and genetic composition. The methicillin-resistant penicillin-binding protein allows for resistance to β-lactam antibiotics and obviates their clinical use during MRSA infections.

In one aspect is a method for treating a subject having a resistant bacterium comprising administering to the subject a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof. In one embodiment, the bacterium is a Gram-positive bacteria. In another embodiment, the Gram-positive bacterium is S. aureus. In further embodiment, the S. aureus is resistant or refractory to a beta-lactam antibiotic. In yet a further embodiment, the beta-lactam antibiotic belongs to the class of penicillins. In a further embodiment, the beta-lactam antibiotic is methicillin. In yet another embodiment, the subject has a methicillin-resistant S. aureus bacteria. In one embodiment the beta-lactam antibiotic is flucloxacillin. In another embodiment is a method for treating a subject having a dicloxacillin-resistant bacteria comprising administering to the subject a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the subject is refractory to dicloxacillin. Also disclosed herein is a method for treating a subject having a methicillin-resistant bacteria comprising administering a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the subject has been determined to have a methicillin-resistant bacteria. In one embodiment the subject is screened for methicillin-resistant bacteria. In another embodiment, the subject screening is performed through a nasal culture. In a further embodiment the methicillin-resistant bacteria is detected by swabbing the nostril(s) of the subject and isolating the bacteria. In another embodiment, Real-time PCR and/or Quantitative PCR is employed to determine whether the subject has a methicillin-resistant bacteria.

In one embodiment is a method for treating a subject having a first-generation cephalosporin-resistant bacteria comprising administering a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the subject is refractory to a first-generation cephalosporin. In one embodiment, the bacteria is resistant to a first-generation cephalosporin. In a further embodiment, the bacteria is resistant to cefacetrile. In another embodiment, the bacteria is resistant to cefadroxil. In yet another embodiment, the bacteria is resistant to cefalexin. In one embodiment, the bacteria is resistant to cefaloglycin. In another embodiment, the bacteria is resistant to cefalonium. In another embodiment, the bacteria is resistant to cefaloridine. In yet another embodiment, the bacteria is resistant to cefalotin. In a further embodiment, the bacteria is resistant to cefapirin. In yet a further embodiment, the bacteria is resistant to cefatrizine. In one embodiment, the bacteria is resistant to cefazaflur. In another embodiment, the bacteria is resistant to cefazedone. In yet another embodiment, the bacteria is resistant to cefazolin. In a further embodiment, the bacteria is resistant to cefradine. In yet a further embodiment, the bacteria is resistant to cefroxadine. In one embodiment, the bacteria is resistant to ceftezole.

In one embodiment is a method for treating a subject having a second-generation cephalosporin-resistant bacteria comprising administering a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the subject is refractory to a second-generation cephalosporin. In another embodiment, the bacteria is resistant to a second-generation cephalosporin. In a further embodiment, the bacteria is resistant to cefaclor. In another embodiment, the bacteria is resistant to cefonicid. In yet another embodiment, the bacteria is resistant to cefprozil. In one embodiment, the bacteria is resistant to cefuroxime. In another embodiment, the bacteria is resistant to cefuzonam. In another embodiment, the bacteria is resistant to cefmetazole. In yet another embodiment, the bacteria is resistant to cefotetan. In a further embodiment, the bacteria is resistant to cefoxitin.

In one embodiment is a method for treating a subject having a third-generation cephalosporin-resistant bacteria comprising administering a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the subject is refractory to a third-generation cephalosporin. In another embodiment, the bacteria is resistant to a third-generation cephalosporin. In a further embodiment, the bacteria is resistant to cefcapene. In another embodiment, the bacteria is resistant to cefdaloxime. In yet another embodiment, the bacteria is resistant to cefdinir. In one embodiment, the bacteria is resistant to cefditoren. In another embodiment, the bacteria is resistant to cefixime. In another embodiment, the bacteria is resistant to cefmenoxime. In yet another embodiment, the bacteria is resistant to cefodizime. In a further embodiment, the bacteria is resistant to cefotaxime. In yet a further embodiment, the bacteria is resistant to cefpimizole. In one embodiment, the bacteria is resistant to cefpodoxime. In another embodiment, the bacteria is resistant to cefteram. In yet another embodiment, the bacteria is resistant to ceftibuten. In a further embodiment, the bacteria is resistant to ceftiofur. In yet a further embodiment, the bacteria is resistant to ceftiolene. In one embodiment, the bacteria is resistant to ceftizoxime. In another embodiment, the bacteria is resistant to ceftriaxone. In yet another embodiment, the bacteria is resistant to cefoperazone. In yet a further embodiment, the bacteria is resistant to ceftazidime.

In one embodiment is a method for treating a subject having a fourth-generation cephalosporin-resistant bacteria comprising administering a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the subject is refractory to a fourth-generation cephalosporin. In another embodiment, the bacteria is resistant to a fourth-generation cephalosporin. In a further embodiment, the bacteria is resistant to cefclidine. In another embodiment, the bacteria is resistant to cefepime. In yet another embodiment, the bacteria is resistant to cefluprenam. In one embodiment, the bacteria is resistant to cefoselis. In another embodiment, the bacteria is resistant to cefozopran. In another embodiment, the bacteria is resistant to cefpirome. In yet another embodiment, the bacteria is refractory to cefquinome.

In one embodiment is a method for treating a subject having a carbapenem-resistant bacteria comprising administering a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the subject is refractory to a carbapenem. In another embodiment, the bacteria is resistant to a carbapenem. In a further embodiment, the bacteria is resistant to imipenem. In another embodiment, the bacteria is resistant to meropenem. In yet another embodiment, the bacteria is resistant to ertapenem. In one embodiment, the bacteria is resistant to faropenem. In another embodiment, the bacteria is resistant to doripenem. In another embodiment, the bacteria is resistant to panipenem. In yet another embodiment, the bacteria is resistant to biapenem,

### Vancomycin-Intermediate and Vancomycin-Resistant Staphylococcus aureus

Vancomycin-intermediate Staphylococcus aureus and vancomycin-resistant staphylococcus aureus are specific types of antimicrobial-resistant Staph bacteria that are refractory to vancomycin treatment. S. aureus isolates for which vancomycin MICs are 4-8 μg/mL are classified as vancomycin-intermediate and isolates for which vancomycin MICs are ≧16 μg/mL are classified as vancomycin-resistant (Clinical and Laboratory Standards Institute/NCCLS. Performance Standards for Antimicrobial Susceptibility Testing. Sixteenth informational supplement. M100-S16. Wayne, Pa.: CLSI, 2006).

As used herein, the term “minimum inhibitory concentration” (MIC) refers to the lowest concentration of an antibiotic that is needed to inhibit growth of a bacterial isolate in vitro. A common method for determining the MIC of an antibiotic is to prepare several tubes containing serial dilutions of the antibiotic, that are then inoculated with the bacterial isolate of interest. The MIC of an antibiotic is determined from the tube with the lowest concentration that shows no turbidity (no growth).

In one aspect is a method of treating a subject having a bacterial infection comprising administering to the subject a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the bacterial infection comprises a vancomycin-intermediate Staphylococcus aureus bacterium. In one embodiment, the vancomycin-intermediate Staphylococcus aureus bacterium has a MIC of between about 4 to about 8 μg/mL. In another embodiment, the vancomycin-intermediate Staphylococcus aureus bacterium has a MIC of about 4 μg/mL. In yet another embodiment, the vancomycin-intermediate Staphylococcus aureus bacterium has a MIC of about 5 μg/mL. In a further embodiment, the vancomycin-intermediate Staphylococcus aureus bacterium has a MIC of about 6 μg/mL. In yet a further embodiment, the vancomycin-intermediate Staphylococcus aureus bacterium has a MIC of about 7 μg/mL. In one embodiment, the vancomycin-intermediate Staphylococcus aureus bacterium has a MIC of about 8 μg/mL.

In another aspect is a method of treating a subject having a bacterial infection comprising administering to the subject a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the bacterial infection comprises a vancomycin-resistant Staphylococcus aureus bacterium. In one embodiment, the vancomycin-resistant Staphylococcus aureus bacterium has a MIC of between about 16 μg/mL. In another embodiment, the vancomycin-resistant Staphylococcus aureus bacterium has a MIC of about ≧16 μg/mL. In yet another embodiment, the vancomycin-resistant Staphylococcus aureus bacterium has a MIC of about 20 μg/mL. In a further embodiment, the vancomycin-resistant Staphylococcus aureus bacterium has a MIC of about 25 μg/mL.

In one embodiment, conditions treated by the compounds described herein include, but are not limited to, endocarditis, osteomyelitis, neningitis, skin and skin structure infections, genitourinary tract infections, abscesses, and necrotizing infections. In another embodiment, the compounds disclosed herein are used to treat conditions, such as, but not limited to, diabetic foot infections, decubitus ulcers, burn infections, animal or human bite wound infections, synergistic-necrotizing gangrene, necrotizing fascilitis, intra-abdominal infection associated with breeching of the intestinal barrier, pelvic infection associated with breeching of the intestinal barrier, aspiration pneumonia, and post-operative wound infections. In another embodiment, the conditions listed herein are caused by, contain, or result in the presence of VISA and/or VRSA.

### Vancomycin-Resistant Enterococci

Enterococci are bacteria that are normally present in the human intestines and in the female genital tract and are often found in the environment. These bacteria sometimes cause infections. In some cases, enterococci have become resistant to vancomycin (also known as vancomycin-resistant enterococci or VRE.) Common forms of resistance to vancomycin occur in enterococcal strains that involve the acquisition of a set of genes endoding proteins that direct peptidoglycan precursors to incorporate D-Ala-D-Lac instead of D-Ala-D-Ala. The six different types of vancomycin resistance shown by enterococcus are: Van-A, Van-B, Van-C, Van-D, Van-E and Van-F. In some cases, Van-A VRE is resistant to both vancomycin and teicoplanin, while in other cases, Van-B VRE is resistant to vancomycin but sensitive to teicoplanin; in further cases Van-C is partly resistant to vancomycin, and sensitive to teicoplanin.

In one aspect, is a method of treating a subject having a vancomycin-resistant enterococci comprising administering to the subject a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the enterococci has developed resistance to vancomycin. In one embodiment, the subject has been previously treated with vancomycin for a sustained period of time. In another embodiment, the subject has been hospitalized. In yet another embodiment, the subject has a weakened immune system such as patients in Intensive Care Units or in cancer or transplant wards. In a further embodiment, the subject has undergone surgical procedures such as, for example, abdominal or chest surgery. In yet a further embodiment, the subject has been colonized with VRE. In one embodiment, the subject has a medical device such that an infection has developed. In another embodiment, the medical device is a urinary catheter or central intravenous (IV) catheter.

In another embodiment, is a method of treating a subject having a vancomycin-resistant enterococci comprising administering to the subject a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the enterococcus has Van-A resistance.

In another embodiment, is a method of treating a subject having a vancomycin-resistant enterococci comprising administering to the subject a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the enterococcus has Van-B resistance.

In another embodiment, is a method of treating a subject having a vancomycin-resistant enterococci comprising administering to the subject a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or a pharmaceutically acceptable salt, ester, solvate, alkylated quaternary ammonium salt, stereoisomer, tautomer or prodrug thereof wherein the enterococcus has Van-C resistance.

### Administration and Pharmaceutical Composition

Pharmaceutical compositions described herein comprise a therapeutically effective amount of a compound described herein (i.e., a compound of any of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′)) formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions described herein can be administered to humans and other animals orally, rectally, parenterally, intracisternally, intravaginally, intraperitoneally, topically (as by powders, ointments, or drops), bucally, or as an oral or nasal spray, or a liquid aerosol or dry powder formulation for inhalation.

Liquid dosage forms for oral administration include pharmaceutically acceptable emulsions, microemulsions, solutions, suspensions, syrups and elixirs. In addition to the active compounds, the liquid dosage forms optionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions are optionally formulated according to the known art using suitable dispersing or wetting agents and suspending agents. The sterile injectable preparation is optionally a sterile injectable solution, suspension or emulsion in a nontoxic parenterally acceptable diluent or solvent, for example, as a solution in 1,3-butanediol. Among the acceptable vehicles and solvents that are optionally employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This is optionally accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is optionally accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are optionally prepared by entrapping the drug in liposomes or microemulsions which are compatible with body tissues.

Compositions for rectal or vaginal administration are preferably suppositories which can be prepared by mixing the compound described herein (i.e., a compound of any of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′)) with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form optionally comprises buffering agents.

Solid compositions of a similar type are optionally employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings known in the pharmaceutical formulating art. They optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type are optionally employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings known in the pharmaceutical formulating art. In such solid dosage forms the active compound is optionally admixed with at least one inert diluent such as sucrose, lactose or starch. Such dosage forms optionally comprise, as is normal practice, additional substances other than inert diluents, e.g., tableting lubricants and other tableting aids such a magnesium stearate and microcrystalline cellulose. In the case of capsules, tablets and pills, the dosage forms optionally comprise buffering agents. They optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as are optionally required. Ophthalmic formulations, ear drops, and the like are also contemplated.

The ointments, pastes, creams and gels may contain, in addition to an active compound described herein, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions described herein are optionally formulated for delivery as a liquid aerosol or inhalable dry powder. Liquid aerosol formulations are optionally nebulized predominantly into particle sizes that can be delivered to the terminal and respiratory bronchioles where bacteria reside in patients with bronchial infections, such as chronic bronchitis and pneumonia. Pathogenic bacteria are commonly present throughout airways down to bronchi, bronchioli and lung parenchema, particularly in terminal and respiratory bronchioles. During exacerbation of infection, bacteria can also be present in alveoli. Liquid aerosol and inhalable dry powder formulations are preferably delivered throughout the endobronchial tree to the terminal bronchioles and eventually to the parenchymal tissue.

Aerosolized formulations described herein are optionally delivered using an aerosol forming device, such as a jet, vibrating porous plate or ultrasonic nebulizer, preferably selected to allow the formation of a aerosol particles having with a mass medium average diameter predominantly between 1 to 5μ. Further, the formulation preferably has balanced osmolarity ionic strength and chloride concentration, and the smallest aerosolizable volume able to deliver effective dose of the compounds described herein compound described herein (i.e., a compound of any of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′)) to the site of the infection. Additionally, the aerosolized formulation preferably does not impair negatively the functionality of the airways and does not cause undesirable side effects.

Aerosolization devices suitable for administration of aerosol formulations described herein include, for example, jet, vibrating porous plate, ultrasonic nebulizers and energized dry powder inhalers, that are able to nebulize the formulation into aerosol particle size predominantly in the size range from 1-5 microns. Predominantly in this application means that at least 70% but preferably more than 90% of all generated aerosol particles are within 1-5 micron range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb™ and AeroDose™ vibrating porous plate nebulizers (AeroGen, Inc., Sunnyvale, Calif.), Sidestream® nebulizers (Medic-Aid Ltd., West Sussex, England), Pari LC® and Pari LC Star® jet nebulizers (Pari Respiratory Equipment, Inc., Richmond, Va.), and Aerosonic™ (DeVilbiss Medizinische Produkte (Deutschland) GmbH, Heiden, Germany) and UltraAire® (Omron Healthcare, Inc., Vernon Hills, Ill.) ultrasonic nebulizers.

In some embodiments, compounds described herein compound described herein (i.e., a compound of any of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′)) are formulated for use as topical powders and sprays that contain, in addition to the compounds described herein, excipients such as lactose, talc, silicic acid, aluminum hydroxide, calcium silicates and polyamide powder, or mixtures of these substances. Sprays optionally contain customary propellants such as hydrofluorocarbons or chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment described herein, bacterial infections are treated or prevented in a patient such as a human or lower mammal by administering to the patient a therapeutically effective amount of a compound described herein, in such amounts and for such time as is necessary to achieve the desired result. By a “therapeutically effective amount” of a compound described herein is meant a sufficient amount of the compound to treat bacterial infections, at a reasonable benefit/risk ratio applicable to any medical treatment. It will be understood, however, that the total daily usage of the compounds and compositions described herein will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors known in the medical arts.

The total daily dose of the compounds described herein compound described herein (i.e., a compound of any of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′)) administered to a human or other mammal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens described herein comprise administration to a patient in need of such treatment from about 10 mg to about 2000 mg of the compound(s) described herein per day in single or multiple doses.

### Synthesis

Compounds disclosed herein are prepared using standard organic synthesis techniques. It will be appreciated that synthetic procedures employed in the preparation of compounds of the invention will depend on the particular substituents present in a compound and that various protection and deprotection may be required as is standard in organic synthesis. In a general synthetic scheme compounds of the invention may be prepared using solution phase or solid phase peptide chemistry techniques. The invention will be more fully understood by reference to the following examples. They should not, however, be construed as limiting the scope of the invention. Abbreviations used herein are as follows:
ELSD: evaporative light scattering detector
DIPEA: diisopropylethylamine
DMAP: 4-dimethylaminopyridine
DMF: dimethylformamide
DCM: dichloromethane
DMSO: dimethyl sulfoxide
EA: ethyl acetate
PE: petroleum ether
TFA: trifluoroacetic acid
TES: triethylsilane
EDC: 1-ethyl-3-(3-dimethylaminopropyl)carbodiimide
HATU: O-(7-azabenzotriazol-1-yl)-N,N,N′,N′-tetramethyluronium hexafluorophosphate
HCTU: O-(6-Chlorobenzotriazol-1-yl)-N,N,N′,N′-tetramethyluronium hexafluorophosphate
HOBt: hydroxybenzotriazole
pyBOP: (Benzotriazol-1-yloxy)tripyrrolidinophosphonium hexafluorophosphate
DMDO: 3,3-Dimethyldioxirane
THF: tetrahydrofuran
MeOH: methanol
EtOAc: ethyl acetate
Trt resin: 2-Chlorotrityl chloride resin
TLC: thin-layer chromatography
5-95 AB, ESI: LC-MS analytical conditions with 100×2.1 mm Acquity BEH C18 column, 1.7 μm particle size, 5% CH₃CN/H₂O, 0.4 min to 95% CH₃CN/H₂O for 5.6 min, with 0.1% formic acid at 40° C.).

Fully protected peptide fragments up to six amino acids in length terminated by a carboxylic acid tail are synthesized on solid phase using chlorotrityl functionalized polystyrene resin (Trt-Cl) and an Fmoc/tBu/Trt/t-Boc protecting group strategy. A representative scheme of a four-amino acid fragment terminated with a carboxylic acid is depicted in Scheme I.

An amino acid is attached to 2-chlorotrityl resin using excess amino acid and DIPEA using DCM as the solvent to afford Compound 1A1. The Fmoc-protecting group is removed by treatment with a solution of 20% piperidine in DMF to afford Compound 1A2. An Fmoc protected amino acid is then attached to the growing peptide by treatment of Fmoc protected amino acid with activating reagents such as a combination of HCTU, HOBT, and DIPEA and addition to the peptide resin to afford Compound 1B1, followed by Fmoc deprotection with 20% piperidine in DMF to afford Compound 1B2. In cases where the coupling partner is a carboxylic acid instead of an amino acid, the Fmoc deprotection is omitted. This process of acid coupling can be repeated to afford Compounds 1C2, 1D2, and 1E1, respectively. Cleavage of the fully protected Compound 1E1 is accomplished by repeated treatment of the resin with 1% TFA in CH₂Cl₂ followed by either aqueous workup of the combined filtrates or removal of the TFA by evaporation under reduced pressure to afford Compound 1F.

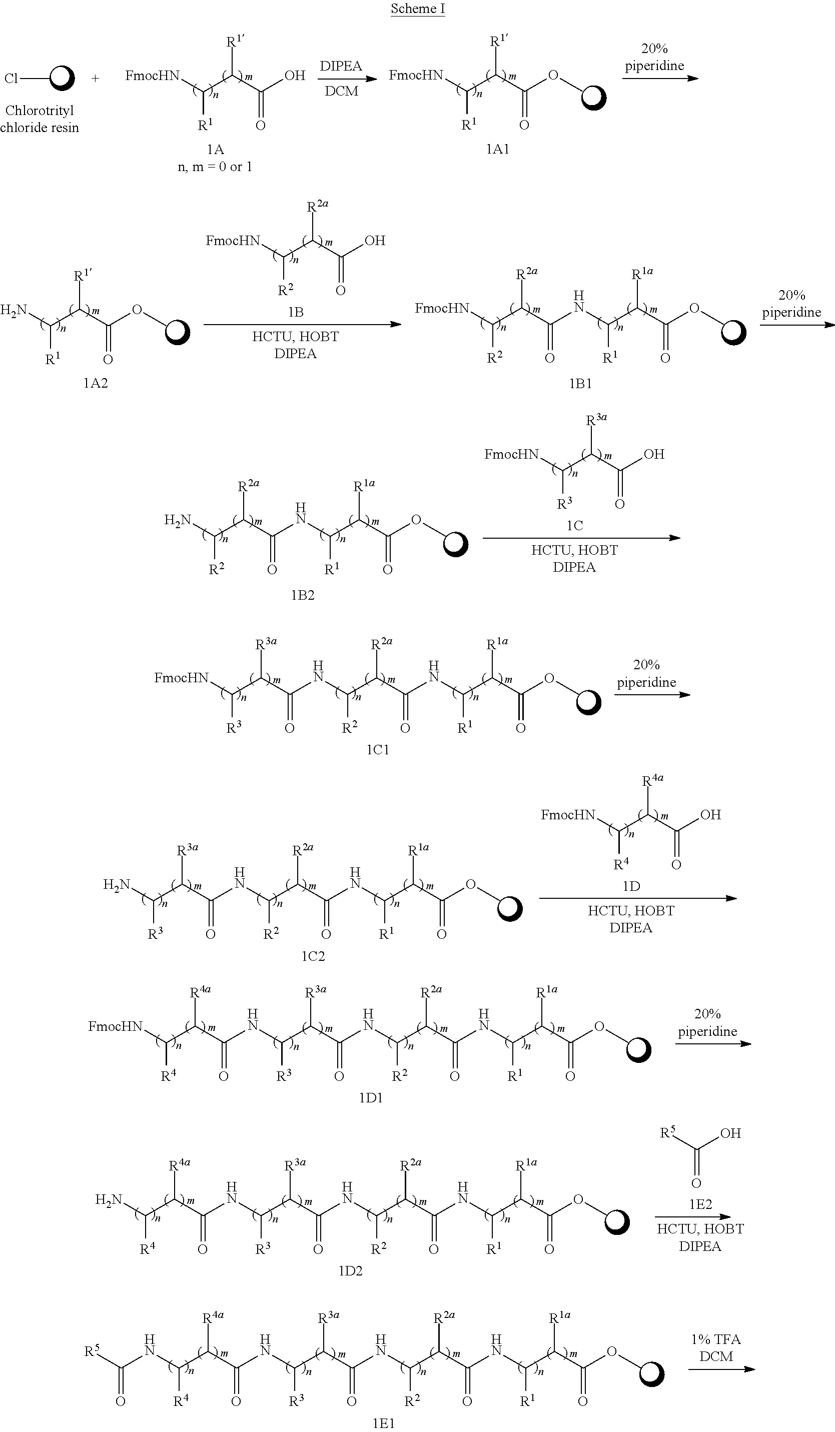

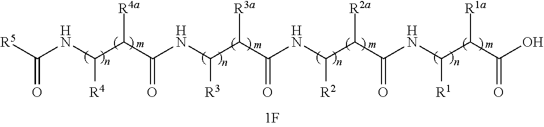

The synthesis of a ketoamide precursor is depicted in Scheme II. Addition to a protected aminoaldehyde with potassium cyanide and NaHSO₃ affords the corresponding cyanohydrin 2B. Hydrolysis of the nitrile with an acid, for example HCl, affords the aminohydroxy acid 2C with concomitant removal of an acid sensitive protecting group such as a Boc-group. Esterification of the hydroxyacid with an acid in the presence of an alcohol, for example HCl in methanol, affords Compound 2D.

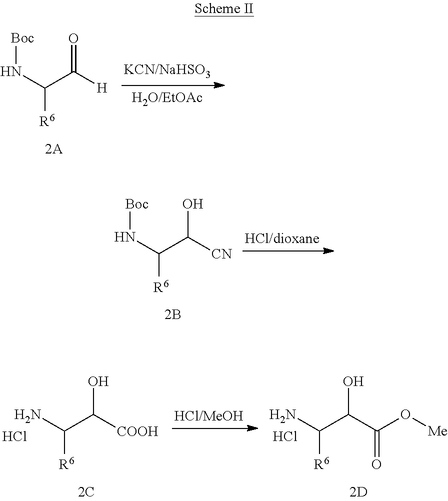

The conversion of peptidic carboxylic acids to ketoamides is depicted in Scheme III. Compound 1F-A is an example peptide that is protected with standard acid-sensitive protecting groups. Peptide coupling of Compound 1F-A with Compound 2D under standard peptide coupling conditions, for example HATU and DIPEA, affords Compound 3. Hydrolysis of the ester under basic conditions, for example K₂CO₃, affords Compound 4. Treatment of Compound 4 with an amine under standard amide coupling conditions, for PyBOP and N-methylmorpholine, affords Compound 5. The alcohol in Compound 5 can be converted to a ketone with an oxidizing agent, for example Dess-Martin periodinane, to afford Compound 6. Global deprotection of the acid-sensitive protecting groups, for example, HCl, affords Compound 7.

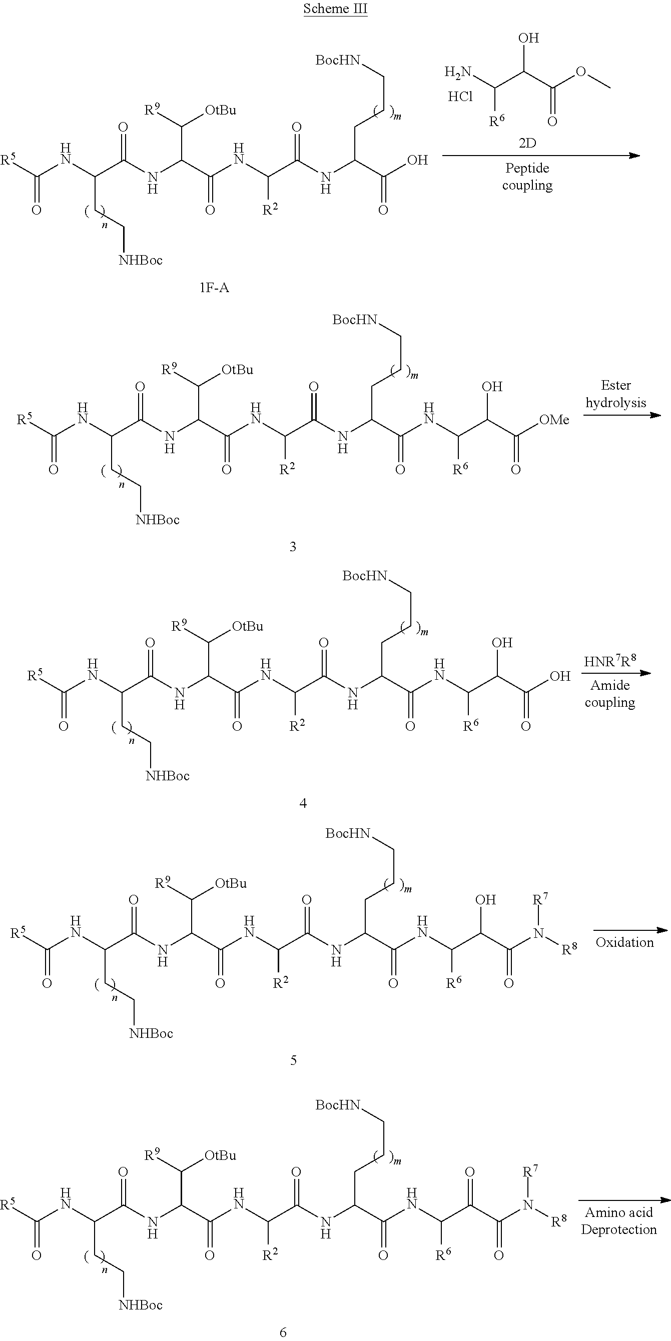

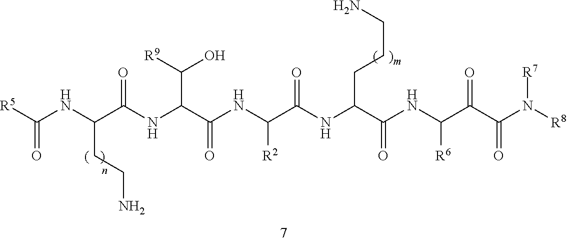

An alternative method to synthesize Compound 7 is depicted in Scheme IV. Compound 1F-A is an example peptide that is protected with standard acid-sensitive protecting groups. Peptide coupling of Compound 1F-A with Compound 2 under standard peptide coupling conditions, for example HATU and DIPEA, affords Compound 5. The alcohol in Compound 5 can be converted to a ketone with an oxidizing agent, for example Dess-Martin periodinane, to afford Compound 6. Global deprotection of the acid-sensitive protecting groups, for example, HCl, affords Compound 7.

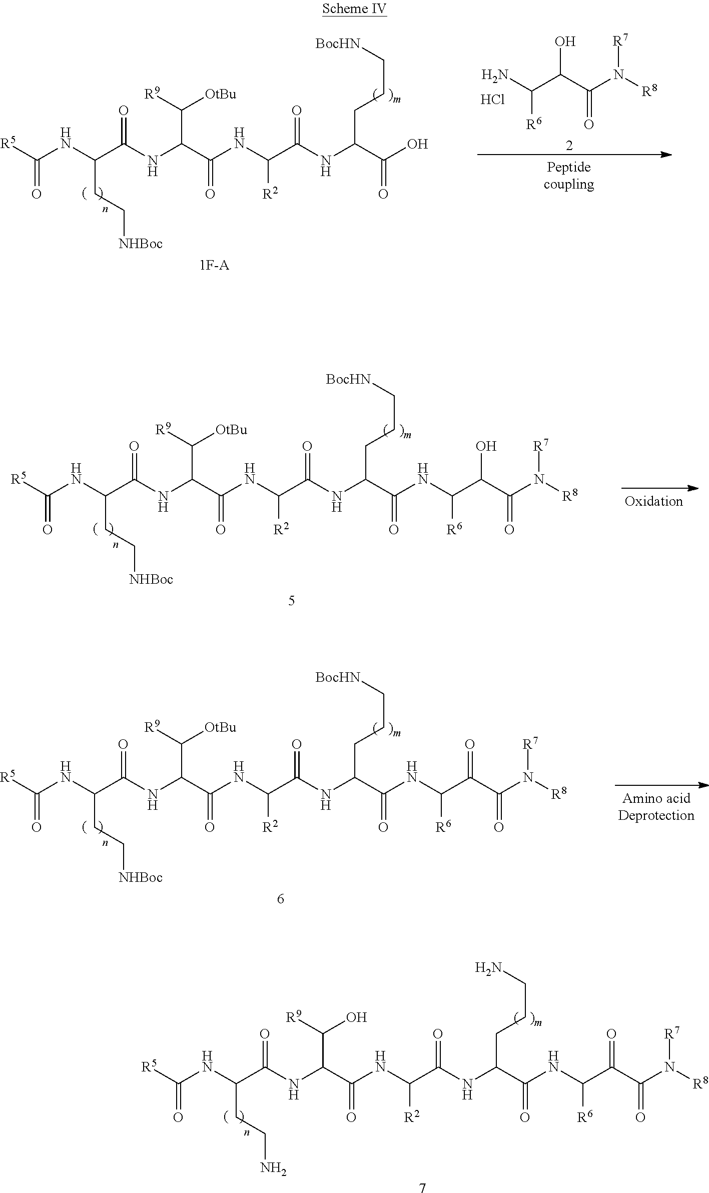

The preparation of Compound 2 is depicted in Scheme V. The addition of nitroethane can be mediated by acid or base, using for example Amberlyst to afford Compound 2E. The nitro group of Compound 2E can be reduced to the corresponding amine with, for example, Raney Nickel and H₂, to afford Compound 2F. Condensation of Compound 2F with an amine, for example methylamine, affords Compound 2.

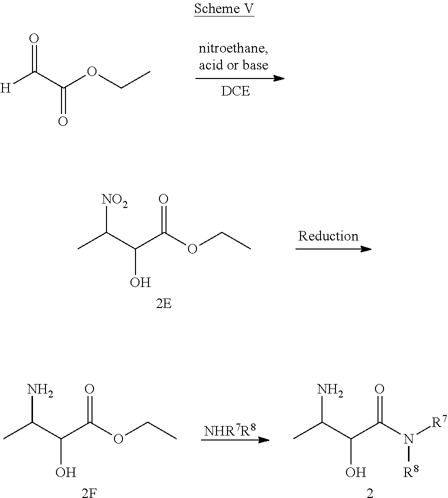

An alternative method to synthesize ketoamides is depicted in Scheme VI. Compound 1F-A is an example peptide that is protected with standard acid-sensitive protecting groups. Coupling of Compound 1F-A with an aminoalcohol under standard peptide coupling conditions, for example HATU and DIPEA, affords Compound 9. Oxidation of the alcohol with, for example, Dess-Martin periodinane, affords Compound 10. Treatment of Compound 10 with an isocyanide, for example, phenethyl isocyanide, in the presence of an acid, for example acetic acid, affords Compound 11. Hydrolysis of the ester group in Compound 11 can be accomplished by treatment with a base, for example potassium carbonate, to afford Compound 12. The alcohol in Compound 12 can be converted to a ketone with an oxidizing agent, for example Dess-Martin periodinane, to afford Compound 13. Global deprotection of the acid-sensitive protecting groups with, for example, TFA, affords Compound 14.

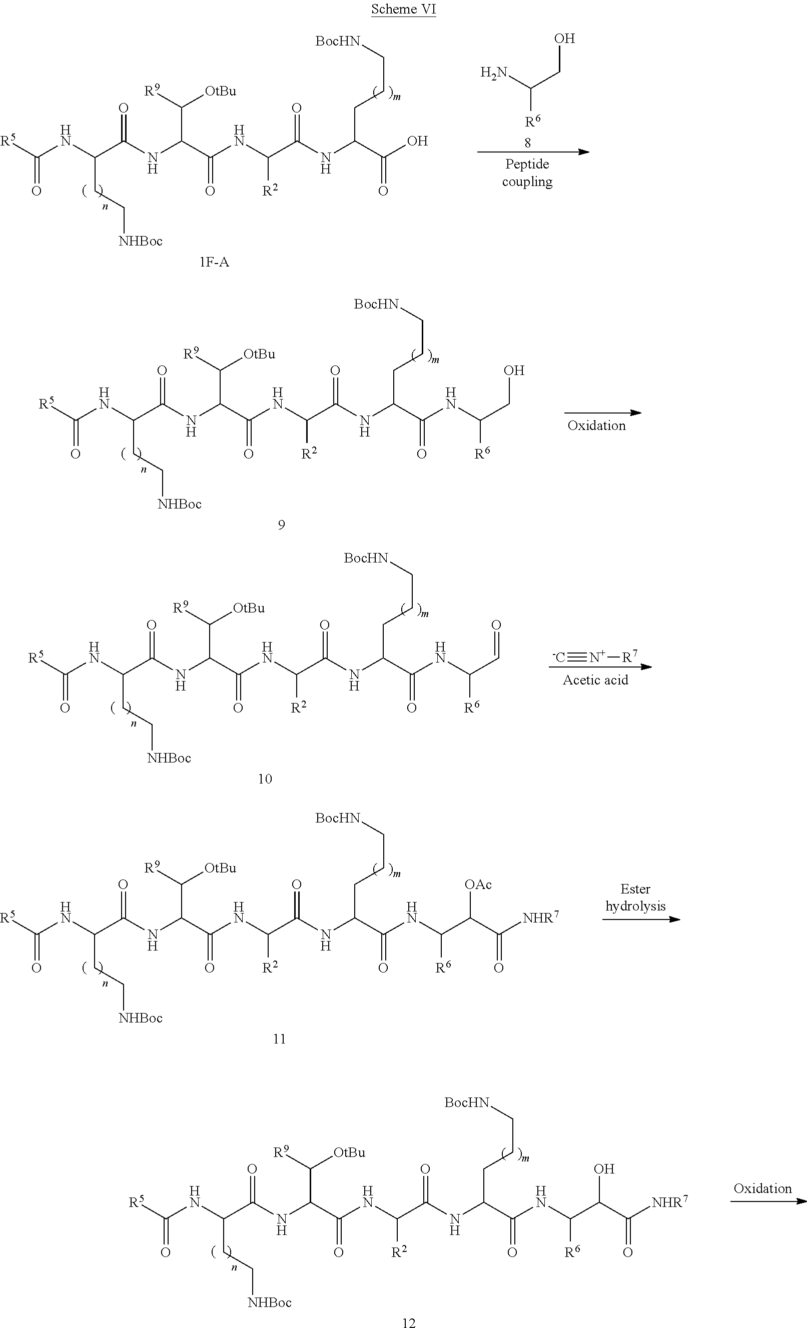

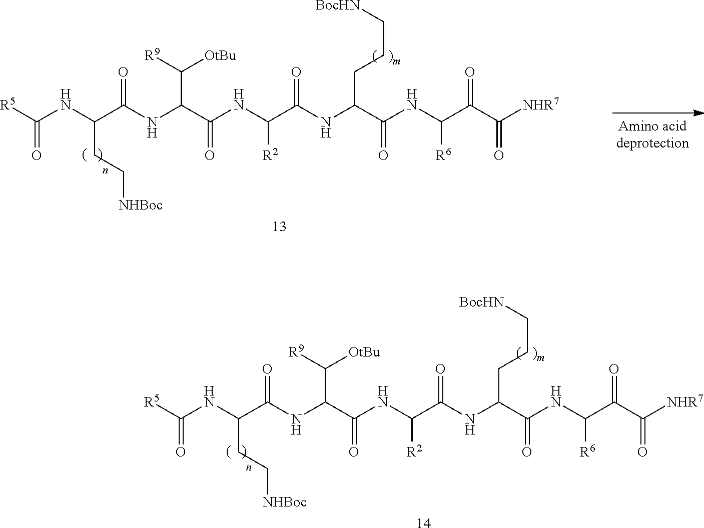

The synthesis of boronate ester peptide is depicted in Scheme VII. An example peptide fragment on resin, for example, Compound 1D1-A, can be synthesized as depicted in Scheme I. The peptide can be cleaved from the resin by treatment with acid, for example, TFA, to afford Compound 15E. Coupling Compound 15E and an amino-alkyl boronate ester under standard peptide coupling conditions, for example HATU and DIPEA, affords Compound 15F. Removal of the Fmoc protecting group can be accomplished by treatment of Compound 15F with piperidine.

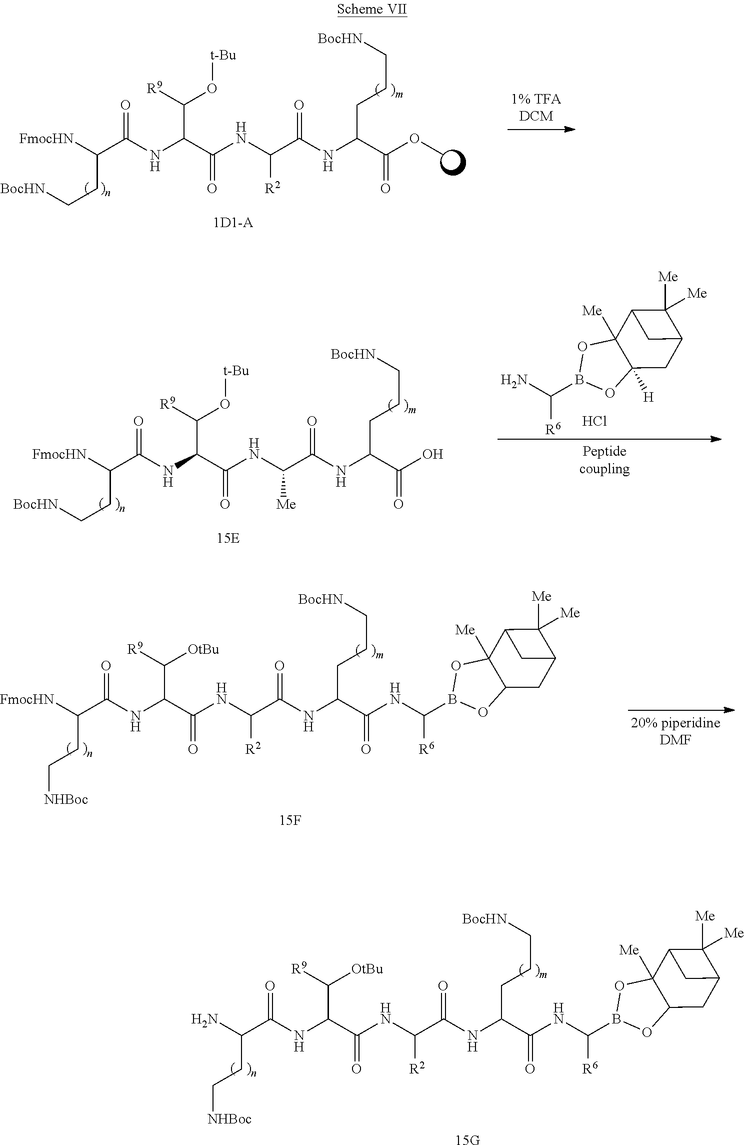

The conversion of boronate ester peptides 15G to lipopeptide boronate esters is depicted in Scheme VIII. Compound 15G can be coupled to an amide under standard coupling conditions, for example EDCI, HOBT and DIPEA to afford Compound 15H. Global deprotection of the acid-sensitive amino acid protecting groups with, for example, TFA, affords Compound 15. Removal of the boronate ester protecting group can be achieved by treatment of Compound 15 with excess of a boronic acid, for example phenylboronic acid in the presence of an acid, to afford Compound 16.

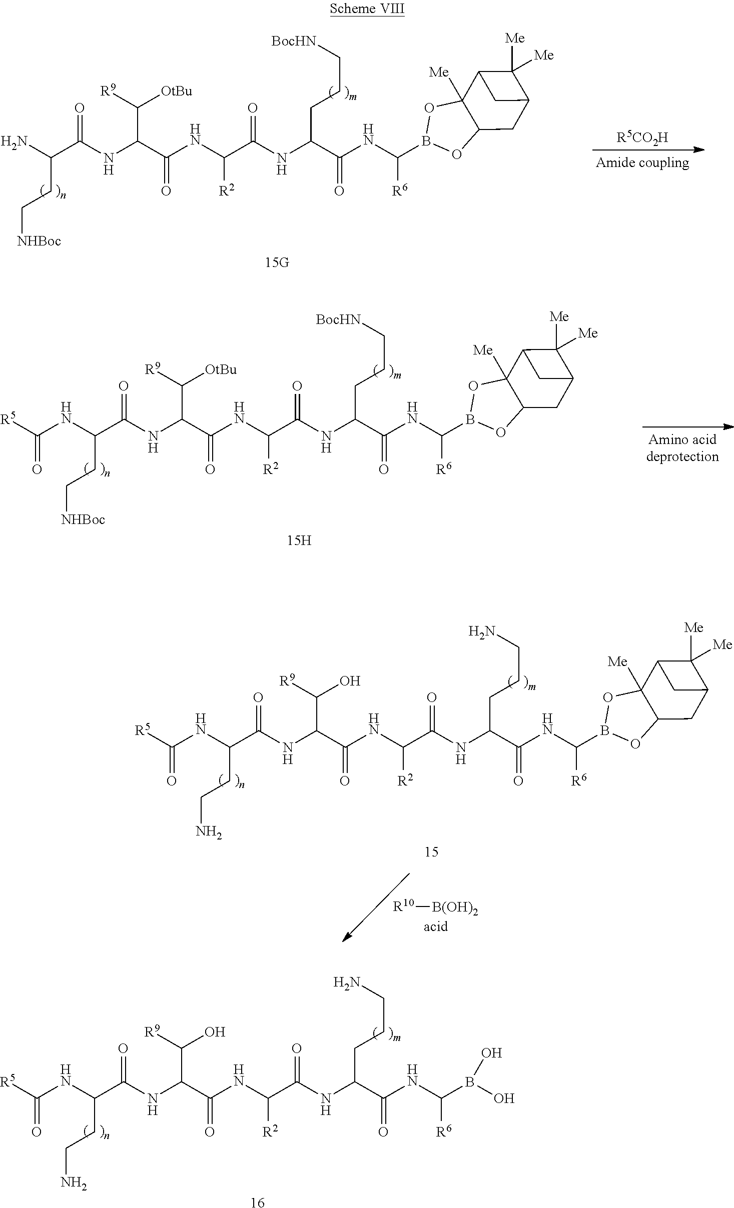

An alternative method to synthesize Compound 15 is depicted in Scheme IX. Amide coupling of Compound 1F-A and an amino-alkyl boronate ester, for example a pinanediol ester under standard peptide coupling conditions, for example HATU and DIPEA, affords Compound 15H. Global deprotection of the acid-sensitive amino acid protecting groups with, for example, TFA, affords Compound 15.

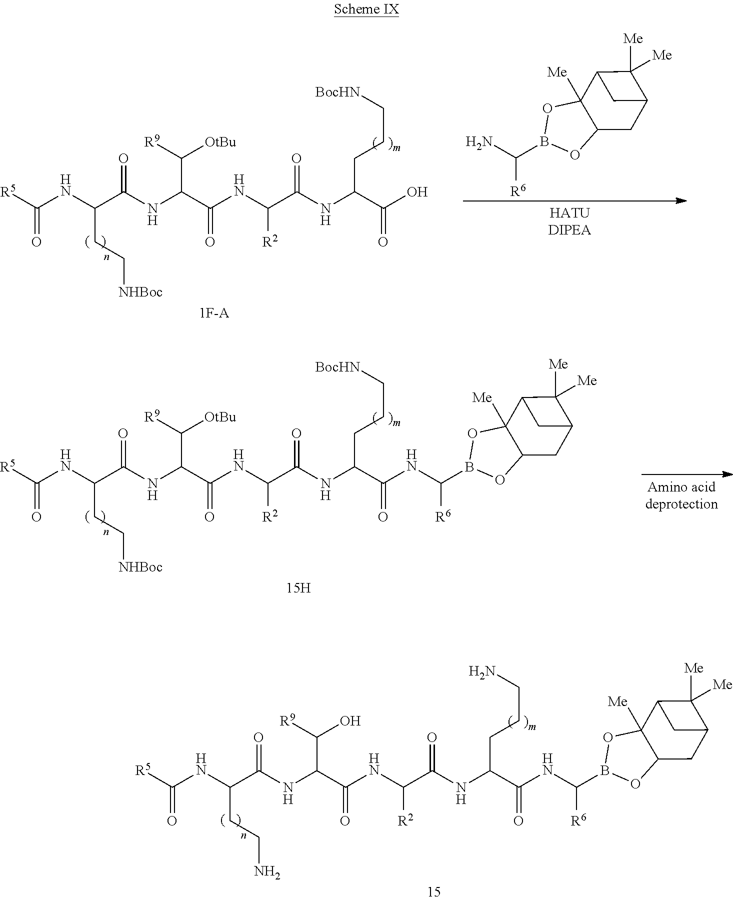

A method to prepare a urea terminal linkage, as in Compound 17, is depicted in Scheme X. An example peptide on resin Compound 1D2-A can be prepared as described in Scheme I. Coupling of Compound 1D2-A and an amine mediated by an di-acylating agent, for example, 1,1′-carbonyldiimidazole, affords Compound 17E. Deprotection from the resin, aminoboronate ester coupling and global peptide deprotection as described in Scheme IX affords Compound 17.

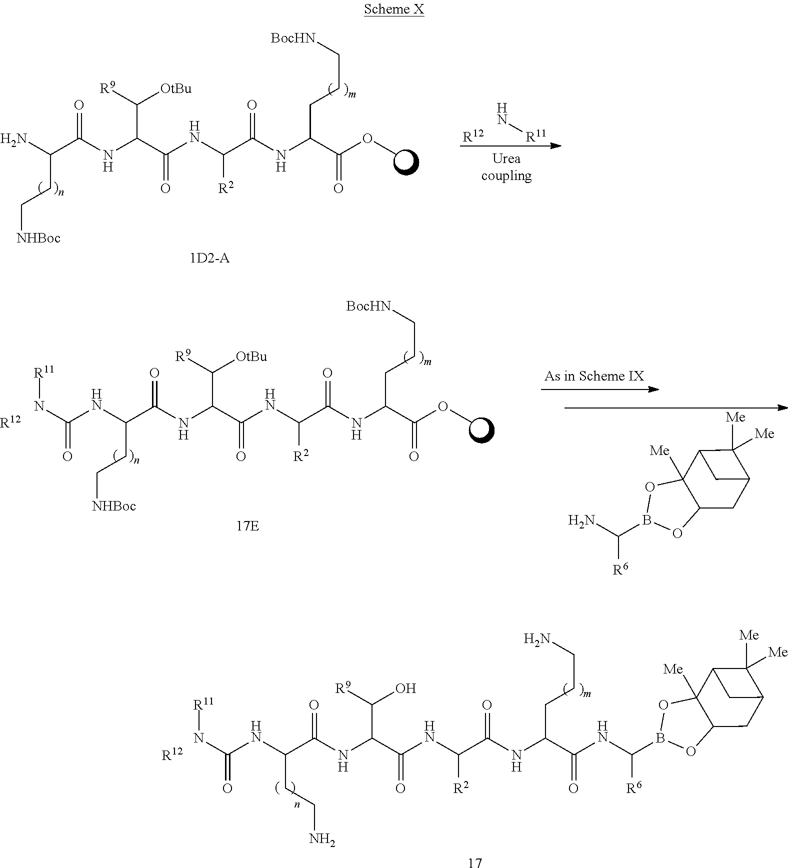

An additional method to prepare a sulfonyl terminal linkage, as in Compound 18, is depicted in Scheme XI. An example peptide on resin Compound 1D2-A can be prepared as described in Scheme I. Coupling of Compound 1D2-A with a sulfonyl chloride mediated by a base, for example triethylamine, affords Compound 18E. Deprotection from the resin, aminoboronate ester coupling and global peptide deprotection as described in Scheme IX affords Compound 18.

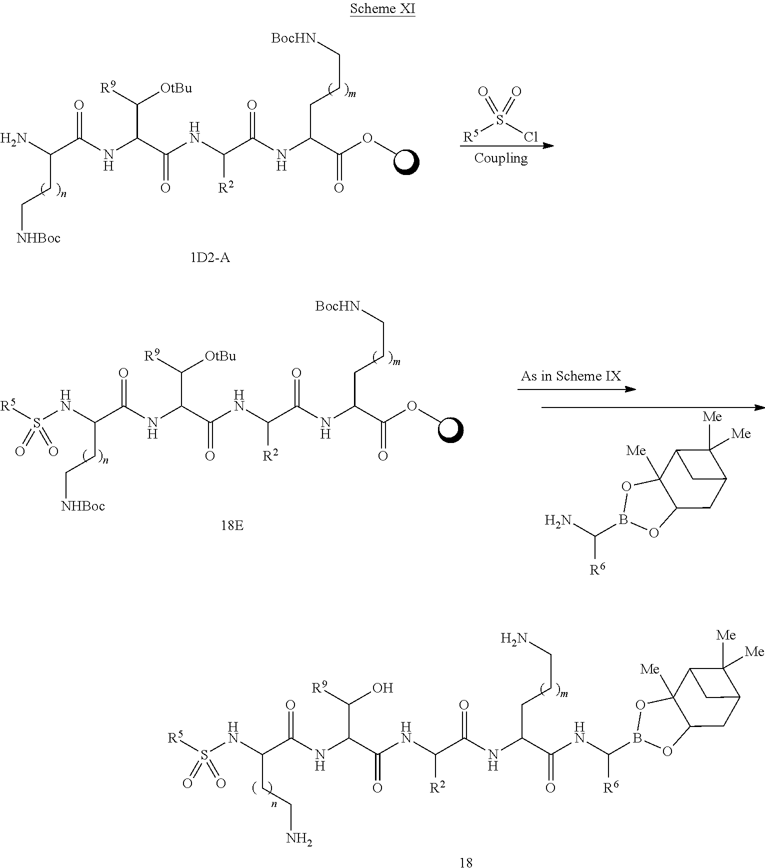

The synthesis of epoxyketone building blocks is depicted in Scheme XII. Addition of an organometallic reagent such as Grignard reagent Compound 20 is added to Weinreb amide 19 to afford olefin Compound 21. Compound 21 can be epoxidized under a number of conditions, for example NaOCl in pyridine, to afford Compound 22. The Boc-protecting group can be removed under acidic conditions, for example TFA in DCM to afford Compound 23.

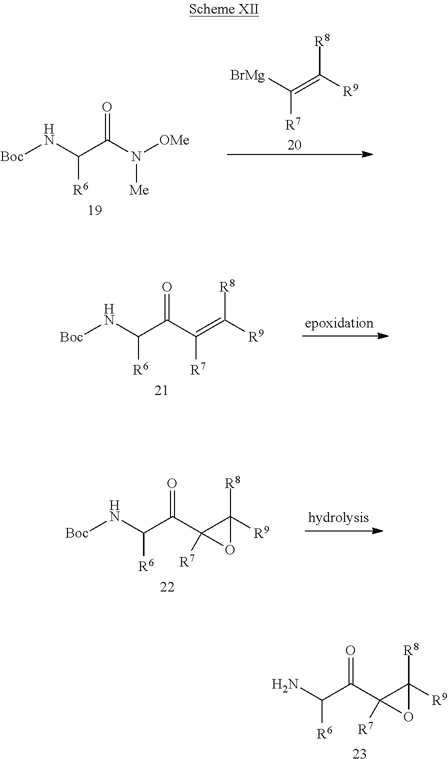

An alternative synthesis of epoxyketone building blocks is depicted in Scheme XIII. Addition of an organometallic reagent such as Grignard reagent 20 is added to aldehyde 2A to afford the allylic alcohol 25. Compound 25 can be epoxidized under a number of conditions, for example VO(acac)₂ and t-butyl hydroperoxide, to afford Compound 26. Oxidation of the alcohol with, for example, Dess-Martin periodinane affords ketone 22. The Boc-protecting group can be removed under acidic conditions, for example TFA in DCM to afford Compound 23.

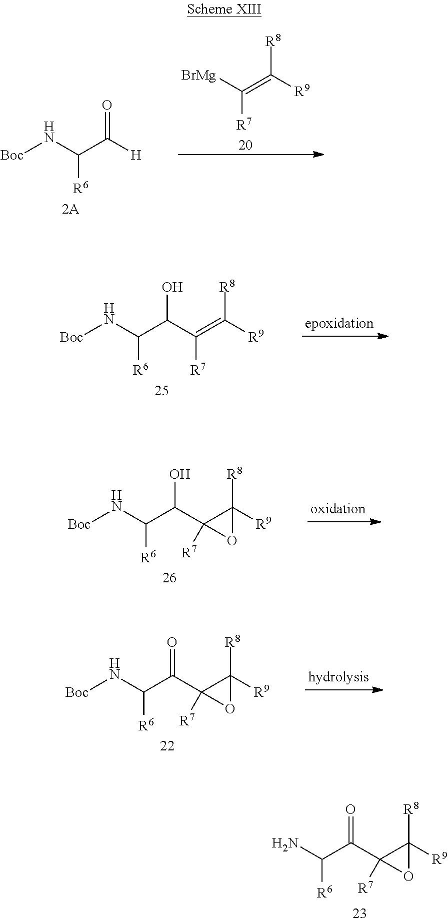

The coupling of an amino-epoxyketone 23 to a protected amino acid is depicted in Scheme XIV. Amide coupling under standard coupling conditions, for example, HATU and DIPEA to afford the coupled peptide followed by protecting group removal to afford the desired Compound 28.

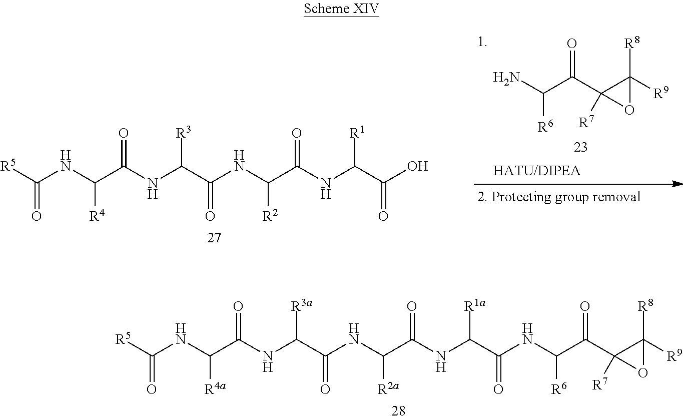

The preparation of a trifluoromethylketone precursor is depicted in Scheme XV. Treatment of an amino-aldehyde with a trifluoromethylating agent, for example TMS-CF₃, in the presence of a fluoride source, such as CsF affords Compound 29. Deprotection of the amino alcohol 29 can be achieved by treatment with acid, for example, trifluoroacetic acid, to afford Compound 30.

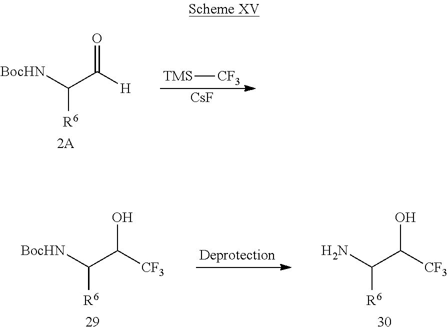

The coupling of an aminoalcohol to a protected amino acid and subsequent conversion to a trifluoromethylketone is depicted in Scheme XVI. Amide coupling with Compound 30 under standard coupling conditions, for example, HATU and DIPEA affords Compound 32. Oxidation of the alcohol with, for example, Dess-Martin periodinane affords ketone 33. Removal of the peptide protecting groups affords Compound 34.

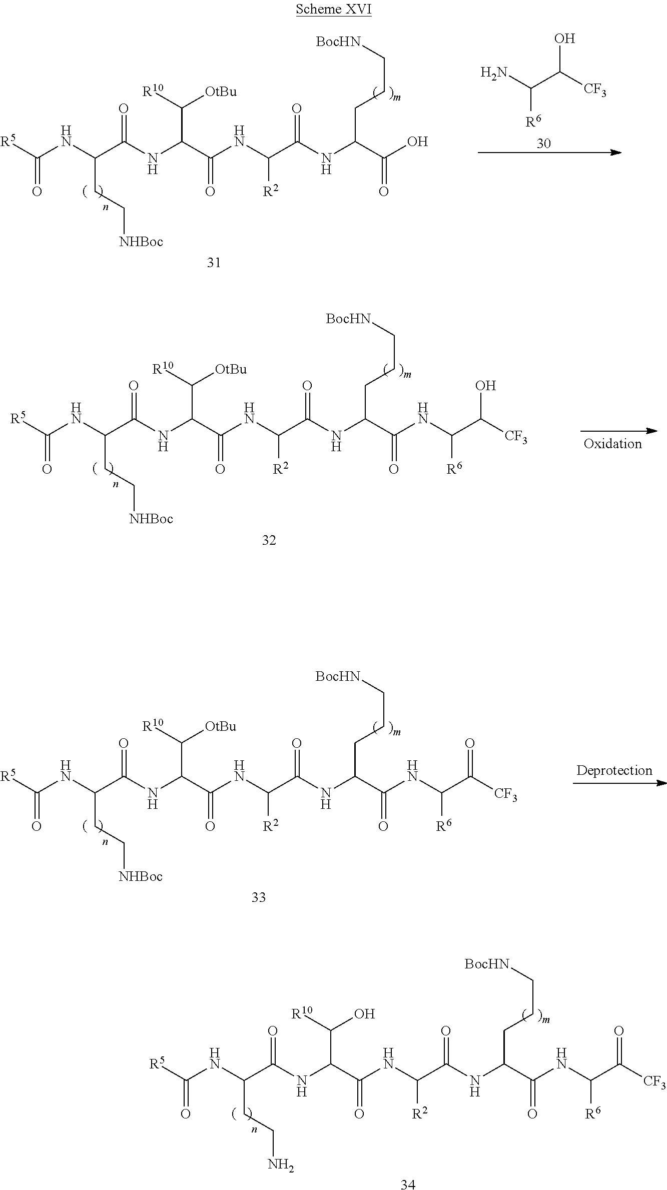

The preparation of Compound 38 is depicted in Scheme XVII. A Boc-amino-chloroketone is deprotected under acidic conditions, for example HCl to afford Compound 36. Amide coupling with Compound 36 under standard coupling conditions, for example, HATU and DIPEA affords Compound 37. Removal of the peptide protecting groups, with HCl in the case of Boc and t-butyl protecting groups, affords Compound 38.

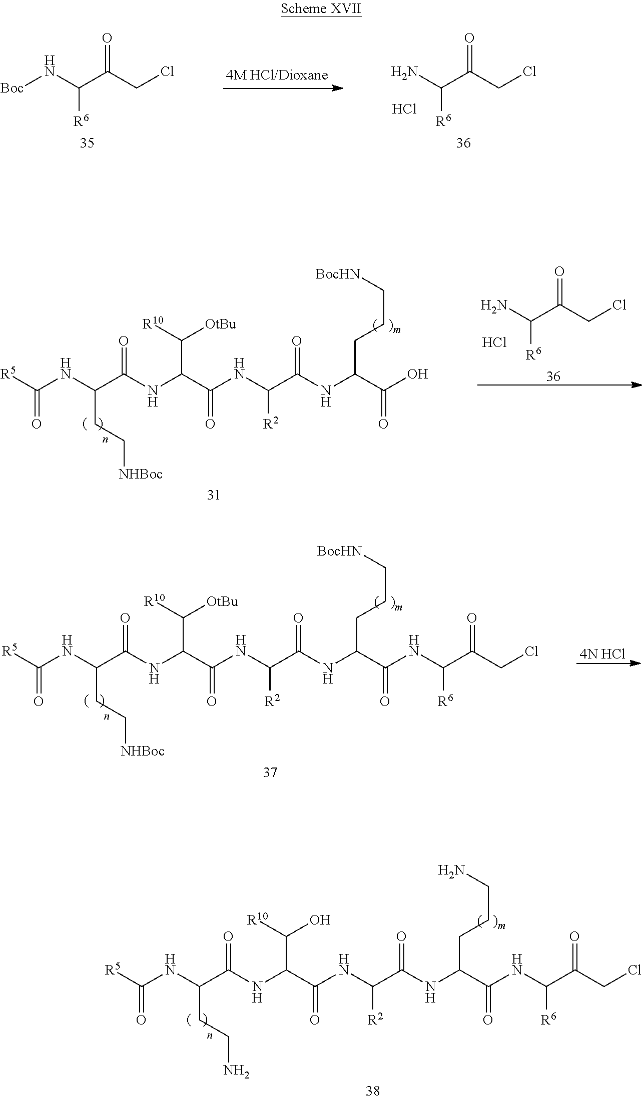

The synthesis of keto-heterocyclic building blocks is depicted in Scheme XVIII. Metallation of heterocycle 39 with an organometallic reagent, for example n-BuLi in hexanes the treatment with MgBr₂, followed by treatment with an aldehyde 2A affords the Compound 40. Oxidation of the alcohol with, for example, Dess-Martin periodinane affords the corresponding ketone 41. The Boc-protecting group can be removed under acidic conditions, for example TFA in DCM, to afford amino-ketone 42.

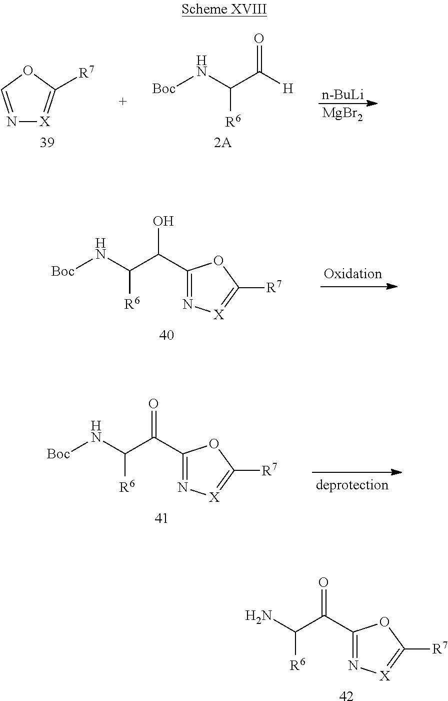

The preparation of compounds of structure 44 is depicted in Scheme XIX. Amide coupling of compound 42 with protected peptide 27 under standard coupling conditions, for example, HATU and DIPEA to affords Compound 43. Removal of the peptide protecting groups affords Compound 44.

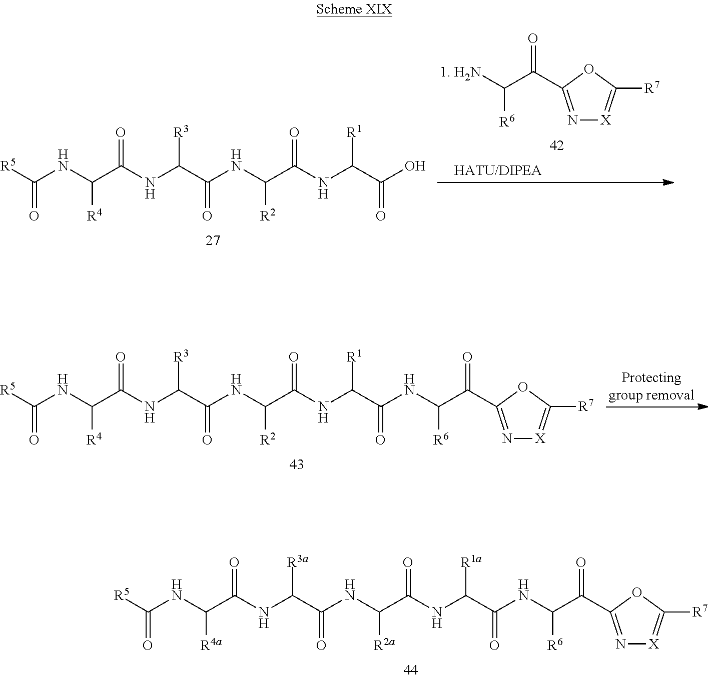

An alternative synthesis of ketoheterocyclic compounds is depicted in Scheme XX. Standard deprotection of a protected amine, with for example TFA in the case of a boc-protected amine affords Compound 45. Amide coupling of Compound 10 on protected peptidic compounds under standard coupling conditions, for example, HATU and DIPEA affords Compound 46. Oxidation of the alcohol with, for example, Dess-Martin periodinane affords the corresponding ketone 43. Removal of the existing peptide protecting groups affords Compound 44.

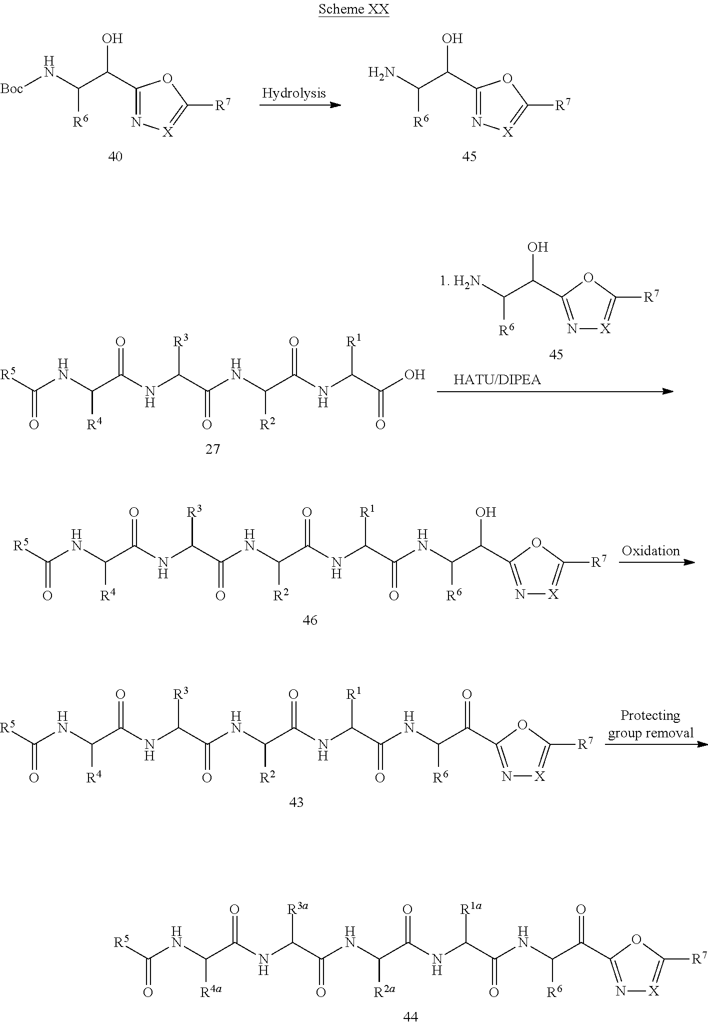

The synthesis of additional keto-heterocyclic building blocks is depicted in Scheme XXI. Metallation of heterocycle 47 with an organometallic reagent, for example n-BuLi in hexanes followed by treatment with an aldehyde 2A affords Compound 48. The Boc-protecting group can be removed under acidic conditions, for example TFA in DCM, to afford amino-alcohol 49.

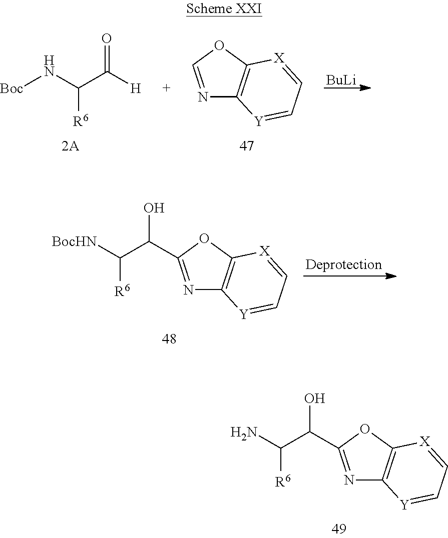

The coupling of an amino-alcohol 49 to a protected amino acid is depicted in Scheme XXII. Amide coupling under standard coupling conditions, for example, HATU and DIPEA to afford the Compound 50. Oxidation of the alcohol with, for example, Dess-Martin periodinane affords the corresponding ketone 51. Removal of the existing peptide protecting groups affords Compound 52.

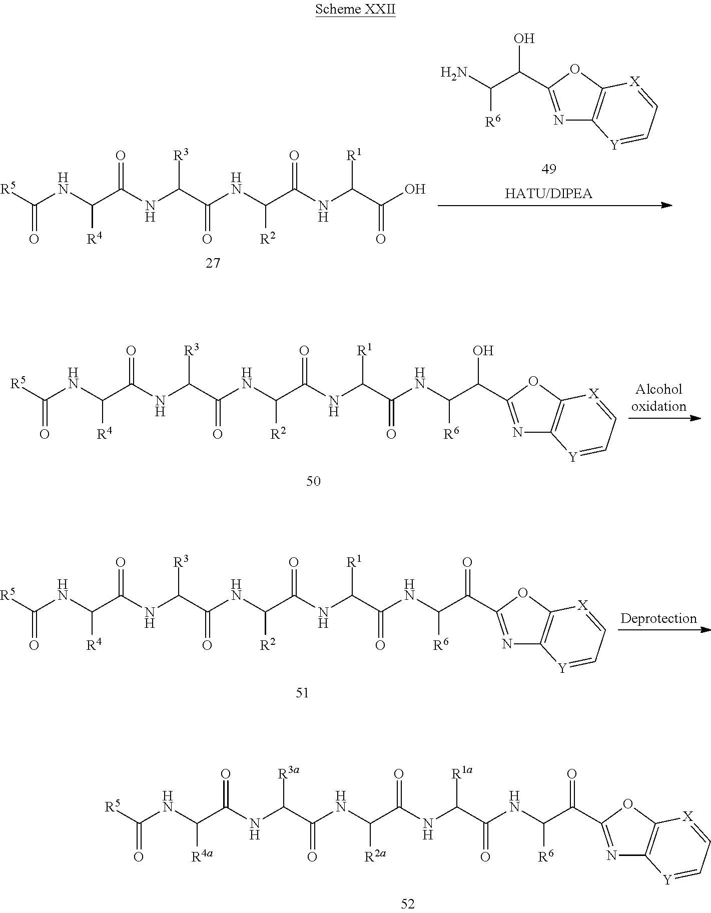

### EXAMPLES

### Example 1

### Preparation of Compound 101

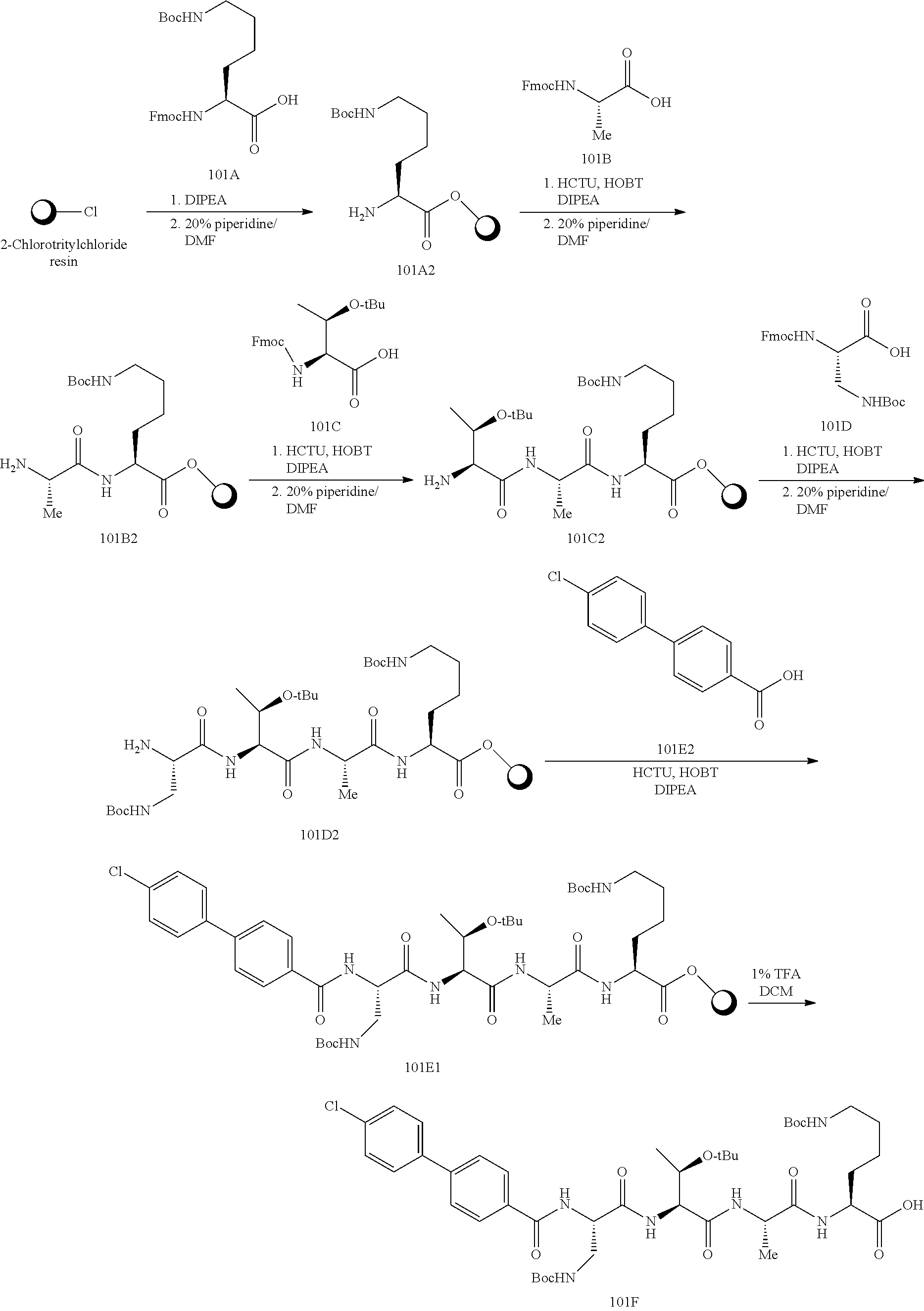

General Method 1.

The preparation of compound 101F utilizes sequential solid phase peptide coupling and subsequent Fmoc-deprotection and is referred to as General Method 1. This is general for any Fmoc-protected amino acid that may contain other protecting groups that are removed under acidic conditions.

To a mixture of Trt resin (1.5 g, 1.5 mmol), and DIPEA (0.77 g, 6.0 mmol) in dry DCM (10 mL) was added a solution of Fmoc-L-Lys(Boc)-OH (2.1 g, 4.5 mmol) in 20 mL dry DCM at 0° C. The mixture was shaken at 25° C. for 5 hr, then the mixture was filtered and the cake was washed with DMF (3×30 mL), DCM (3×30 mL) and MeOH (3×30 mL, to quench the possible unreacted trityl resin). To the above resin was added approximately 20% piperidine/DMF (70 mL) to remove the Fmoc group. The mixture was shaken for 10 min and repeated three times. The mixture was then washed with DMF (3×30 mL) and DCM (2×30 mL) to give compound 101A2.

A mixture of Fmoc-L-Ala-OH (1.4 g, 4.5 mmol), HCTU (1.86 g, 4.5 mmol), HOBT (0.61 g, 4.5 mmol) and DIPEA (0.58 g, 4.5 mmol) in dry DMF (20 mL) was stirred at 25° C. for 30 mins. Then the above mixture was added to compound 101A2 (1.5 mmol) in 30 mL DMF and shaken at 25° C. for 5 hrs. After LCMS showed the reaction was completed, the mixture was filtered and the residue was washed with DMF (3×30 mL) and DCM (3×30 mL). To the above resin was added approximately 30 mL 20% piperidine/DMF to remove the Fmoc group. The mixture was shaken for 10 min and repeated for three times. The mixture was then washed with DMF (3×30 mL) and DCM (2×30 mL) to give compound 101B2.

A mixture of Fmoc-L-Thr(tBu)-OH (1.8 g, 4.5 mmol), HCTU (1.86 g, 4.5 mmol), HOBT (0.61 g, 4.5 mmol) and DIPEA (0.58 g, 4.5 mmol) in dry DMF (20 mL) was stirred at 25° C. for 20 mins. Then the above mixture was added to compound 101B2 (1.5 mmol) and shaken at 25° C. for 5 hrs. After LCMS showed the reaction was completed, the mixture was filtered and the residue was washed with DMF (3×30 mL) and DCM (3×30 mL). To the above resin was added approximately 30 mL 20% piperidine/DMF to remove the Fmoc group. The mixture was shaken for 10 mins and repeated for three times. The mixture was then washed with DMF (3×30 mL) and DCM (2×30 mL) to give compound 101C2.

A mixture of Fmoc-L-Dap(Boc)-OH (1.92 g, 4.5 mmol), HCTU (1.86 g, 4.5 mmol), HOBT (0.61 g, 4.5 mmol) and DIPEA (0.58 g, 4.5 mmol) in dry DMF (20 mL) was stirred at 25° C. for 20 mins. Then the above mixture was added to compound 101C2 (1.5 mmol) and shaken at 25° C. for 5 hrs. After LCMS showed the reaction was completed, the mixture was filtered and the residue was washed with DMF (3×30 mL) and DCM (3×30 mL). To the above resin was added approximately 30 mL 20% piperidine/DMF to remove the Fmoc group. The mixture was shaken for 10 min and repeated for three times. The mixture was then was washed with DMF (3×30 mL) and DCM (2×30 mL) to give compound 101D2.

A mixture of 4-(4-chlorophenyl)benzoic acid (4.5 mmol), HCTU (1.86 g, 4.5 mmol), HOBT (0.61 g, 4.5 mmol) and DIPEA (0.58 g, 4.5 mmol) in dry DMF (20 mL) was stirred at 20° C. for 30 min. Then the above mixture was added to compound 101D2 (1.5 mmol) and shaken at 20° C. for 5 hrs. After LCMS showed the reaction was completed, the mixture was filtered and the residue was washed with DMF (3×30 mL) and DCM (3×30 mL) to give compound 101E1.

A mixture of compound 101E1 (1.5 mmol) was treated with 1% TFA/DCM (4 mL) for 5 min and filtered. This operation was repeated three times. The filtrate was treated with saturated NaHCO₃ solution until pH ˜7-8. The aqueous layer was adjusted to pH ˜3-4 with citric acid. The mixture was extracted with DCM (8 mL) three times, and then the combined organic layers were washed with brine, dried over Na₂SO₄ and concentrated to give 0.65 g of compound 101F. MS (ESI) m/z 875.1 (M+H)⁺.

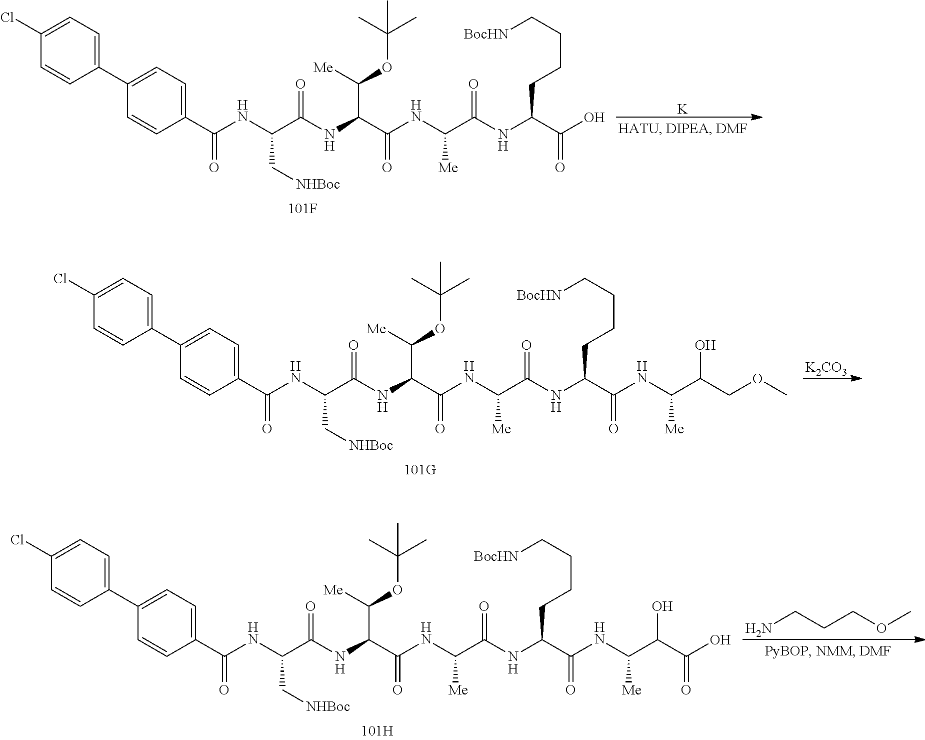

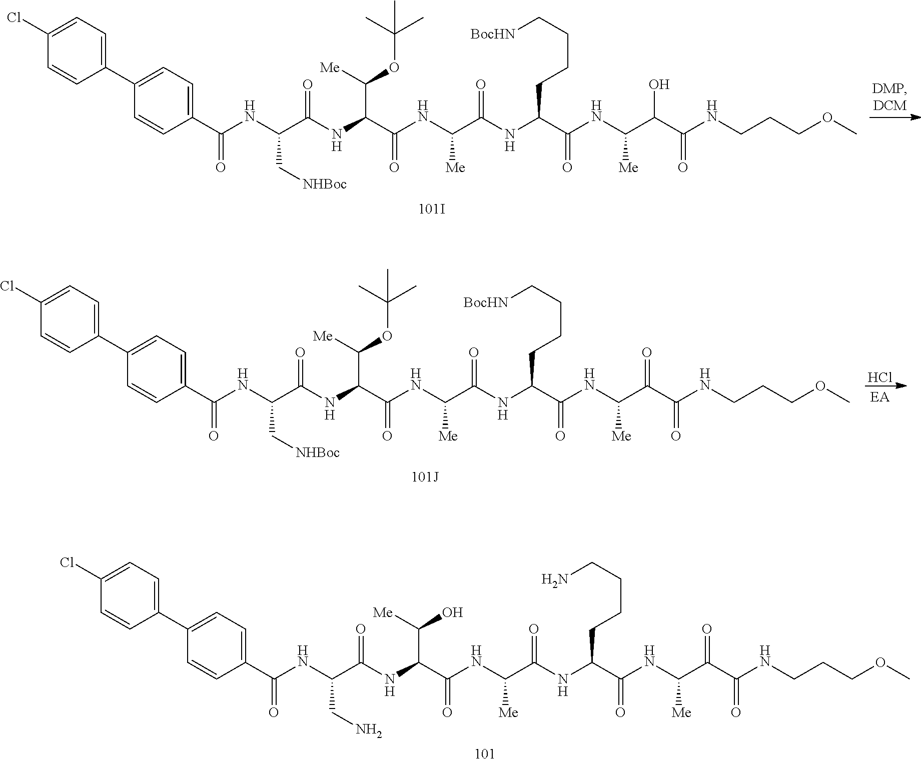 [SMILES: COCCCNC(=O)C(O)[C@H](C)NC(=O)[C@H](CCCCNC(=O)OC(C)(C)C)NC(=O)[C@H](C)NC(=O)[C@@H](NC(=O)[C@H](CNC(=O)OC(C)(C)C)NC(=O)c1ccc(-c2ccc(Cl)cc2)cc1)[C@@H](C)OC(C)(C)C ; COCCCNC(=O)C(=O)[C@H](C)NC(=O)[C@H](CCCCNC(=O)OC(C)(C)C)NC(=O)[C@H](C)NC(=O)[C@@H](NC(=O)[C@H](CNC(=O)OC(C)(C)C)NC(=O)c1ccc(-c2ccc(Cl)cc2)cc1)[C@@H](C)OC(C)(C)C ; Cl ; COCCCNC(=O)C(=O)[C@H](C)NC(=O)[C@H](CCCCN)NC(=O)[C@H](C)NC(=O)[C@@H](NC(=O)[C@H](CN)NC(=O)c1ccc(-c2ccc(Cl)cc2)cc1)[C@@H](C)O]

General Method 2.

The peptide coupling of 101F with K, followed by deprotection, amine coupling, oxidation and sidechain deprotection to give Compound 101 is referred to as General Method 2.

A mixture of compound 101F (3.0 g, 3.43 mmol), compound K (1.74 g, 10.28 mmol) and DIPEA (2.21 g, 17.13 mmol) in N,N-dimethylformamide (25 mL) was stirred at 0° C. for 5 min. Then HATU (1.99 g, 6.86 mmol) was added to the mixture and stirred at room temperature for 12 h. The reaction mixture was poured into ice-water (80 mL), and the suspension was filtered. The cake was washed with water (40 mL*3) and dried under reduced pressure to give the crude compound 101G, which was purified by silica gel column (eluting with 5% to 10% methanol in dichloromethane) to give compound 101G (2.5 g, 74% yield) as a mixture of diastereomers.

A solution of compound 101G (2.1 g, 2.12 mmol) in methanol (60 mL) was stirred at 0° C., to which K₂CO₃ (2.93 g, 21.20 mmol) dissolved in water (60 mL) was added. The reaction mixture was stirred at room temperature for 18 h and concentrated under reduced pressure. The residue was adjusted with HCl (1N) to pH 4˜5 and extracted with dichloromethane (100 mL*3). The combined dichloromethane was washed with water (50 mL*3), brine (50 mL*3), dried over Na₂SO₄, filtered and concentrated under reduced pressure to give the desired compound 101H (2.0 g, 97% yield) as a mixture of diastereomers.

A mixture of compound 101H (400 mg, 0.41 mmol), 3-methoxypropan-1-amine (73 mg, 0.82 mmol) and 4-methylmorpholine (124 mg, 1.23 mmol) in N,N-dimethylformamide (5 mL) was stirred at 0° C. for 5 min. ByBOP (426 mg, 0.82 mmol) was added and the mixture was stirred at room temperature for 12 h. The reaction mixture was poured into ice-water (20 mL) and filtered. The cake was washed with water (20 mL*3) and dried under reduced pressure to give the desired compound 5 (300 mg, 85% purity) as a white solid, which was used directly without further purification. Compound 1011 (300 mg, 0.287 mmol) was dissolved in anhydrous dichloromethane (10 mL) and stirred at 0° C. under nitrogen. To this homogeneous solution, Dess-Martin reagent (243.3 mg, 0.574 mmol) was added in one portion at 0° C. The reaction was stirred at 0° C. for an hour and then at 27° C. for another 12 hours. The reaction mixture was diluted with water (20 mL) and EtOAc (100 mL). The separated organic layer was washed with saturated Na₂S₂O₃ (20 mL*3), Na₂CO₃ (20 mL*3), brine (30 mL*3) and dried over Na₂SO₄. The mixture was filtered and concentrated under reduced pressure to give a residue which was purified by silica gel chromatography (eluting with 2% to 5% methanol in dichloromethane) to give compound 101J (180 mg, 85% purity).

A mixture of compound 101J (180 mg, 0.172 mmol) in HCl/EtOAc (5 mL, 4 M/L) was stirred at room temperature for 2 h. The reaction mixture was concentrated under reduced pressure to give the crude product which was purified by a reverse-phase preparatory HPLC to give compound 101 (30 mg, 22.1% yield) as the hydrochloride salt. MS (ESI) for (C₃₇H₅₃ClN₈O₉): m/z 789.3 [M+H]⁺. ¹HNMR (CD₃OD, 400 MHz): δ 8.03 (d, J=8.4, 2H), 7.76 (d, J=8.4, 2H), 7.67 (d, J=8.4, 2H), 7.47 (d, J=8.4, 2H), 5.03-5.05 (m, 1H), 4.30-4.37 (m, 5H), 3.57-3.66 (m, 1H), 3.41-3.44 (m, 3H), 3.31 (m, 4H), 3.26 (m, 1H), 2.91-2.95 (m, 2H), 1.76-1.79 (m, 5H), 1.40-1.46 (m, 5H), 1.18-1.20 (m, 3H), 1.10-1.12 (m, 2H).

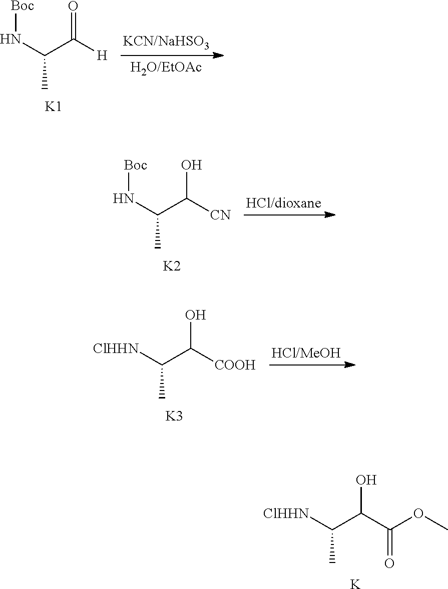

Aldehyde K1 (10.0 g, 57.73 mmol) in H₂O (120 mL) was treated with NaHSO₃ (6.0 g, 57.73 mmol) in 40 mL of H₂O. EtOAc (800 mL) and KCN (3.76 g, 57.73 mmol) in H₂O (40 mL) were added to the above mixture. The reaction mixture was stirred at 24° C. for 10 h. The EtOAc layer was washed with brine (100 mL*3) and dried over anhydrous Na₂SO₄. The mixture was filtered and concentrated under reduced pressure to give compound K2 (10.8 g, 93% yield) as a white solid.

To a solution of K2 (10.8 g, 54 mmol) in 30 mL of dioxane was added HCl (conc.) (30 mL) at 25° C. The reaction mixture was stirred at 100° C. for 10 h and concentrated under reduced pressure. 6.8 g of K3 was obtained as a brown oil and used for next step without further purification. A solution of K3 (6.8 g, crude) in HCl/MeOH (4M, 30 mL) was stirred at room temperature for 16 h. The resulting mixture was concentrated under reduced pressure. 7.2 g of the desired product K was obtained as red oil, which was used without further purification.

### Example 2

### Preparation of Compound 102

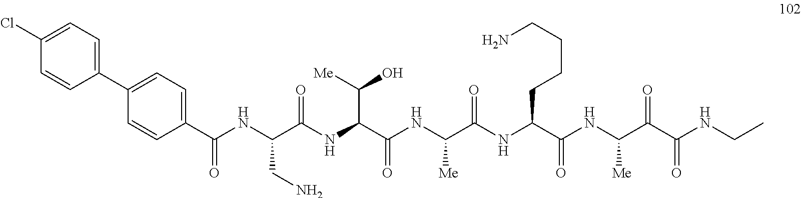 [SMILES: CCNC(=O)C(=O)[C@H](C)NC(=O)[C@H](CCCCN)NC(=O)[C@H](C)NC(=O)[C@@H](NC(=O)[C@H](CN)NC(=O)c1ccc(-c2ccc(Cl)cc2)cc1)[C@@H](C)O]

Compound 102 was prepared according to General Methods 1 and 2 substituting ethylamine for 3-methoxypropan-1-amine. (24.9 mg, 27.6% yield). MS (ESI) for (C₃₅H₄₉ClN₈O₈): m/z 745.4 [M+H]⁺. ¹H NMR (400 MHz, CD₃OD) δ: 1.10-1.13 (m, 9H), 1.19-1.20 (m, 6H), 1.41-1.43 (m, 3H), 1.65-1.66 (m, 1H), 2.91-2.95 (m, 2H), 3.20-3.26 (m, 2H), 3.44-3.57 (m, 2H), 4.30-4.38 (m, 5H), 5.04-5.10 (m, 2H), 7.46 (d, J=8.8 Hz, 2H), 7.66 (d, J=8.4 Hz, 2H), 7.74 (d, J=8.4 Hz, 2H), 8.04 (d, J=8.0 Hz, 2H).

### Example 3

### Preparation of Compound 103

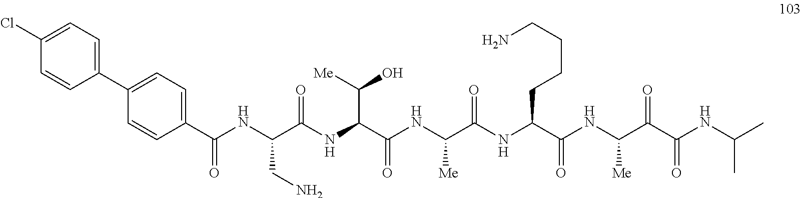 [SMILES: CC(C)NC(=O)C(=O)[C@H](C)NC(=O)[C@H](CCCCN)NC(=O)[C@H](C)NC(=O)[C@@H](NC(=O)[C@H](CN)NC(=O)c1ccc(-c2ccc(Cl)cc2)cc1)[C@@H](C)O]

Compound 103 was prepared according to General Methods 1 and 2 substituting isopropylamine for 3-methoxypropan-1-amine. (75.9 mg, 39.5% yield). MS (ESI) for (C₃₆H₅₁ClN₈O₈): m/z 759.1 [M+H]⁺. ¹H NMR (400 MHz, CD₃OD) δ 1.07-1.21 (m, 10H), 1.45-1.47 (m, 6H), 1.68-1.87 (m, 4H), 2.96 (t, J=6.8 Hz, 2H), 3.48-3.61 (m, 2H), 4.00-4.01 (m, 1H), 4.23-4.40 (m, 5H), 5.08-5.11 (m, 1H), 7.50 (d, J=8.4 Hz, 2H), 7.70 (d, J=8.0 Hz, 2H), 7.79 (d, J=8.0 Hz, 2H), 8.08 (d, J=8.0 Hz, 2H).

### Example 4

### Preparation of Compound 104

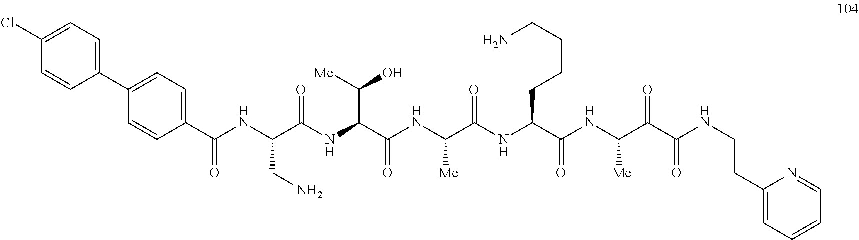 [SMILES: C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)C(=O)NCCc1ccccn1]

Compound 104 was prepared according to General Methods 1 and 2 substituting 2-(pyridin-2-yl)ethanamine for 3-methoxypropan-1-amine. (24.9 mg, 27.6% yield). MS (ESI) for (C₃₅H₄₉ClN₈O₈): m/z 745.4 [M+H]⁺. ¹H NMR (400 MHz, CD₃OD) δ 1.10-1.13 (m, 9H), 1.19-1.20 (m, 6H), 1.41-1.43 (m, 3H), 1.65-1.66 (m, 1H), 2.91-2.95 (m, 2H), 3.20-3.26 (m, 2H), 3.44-3.57 (m, 2H), 4.30-4.38 (m, 5H), 5.04-5.10 (m, 2H), 7.46 (d, J=8.8 Hz, 2H), 7.66 (d, J=8.4 Hz, 2H), 7.74 (d, J=8.4 Hz, 2H), 8.04 (d, J=8.0 Hz, 2H).

### Example 5

### Preparation of Compound 105

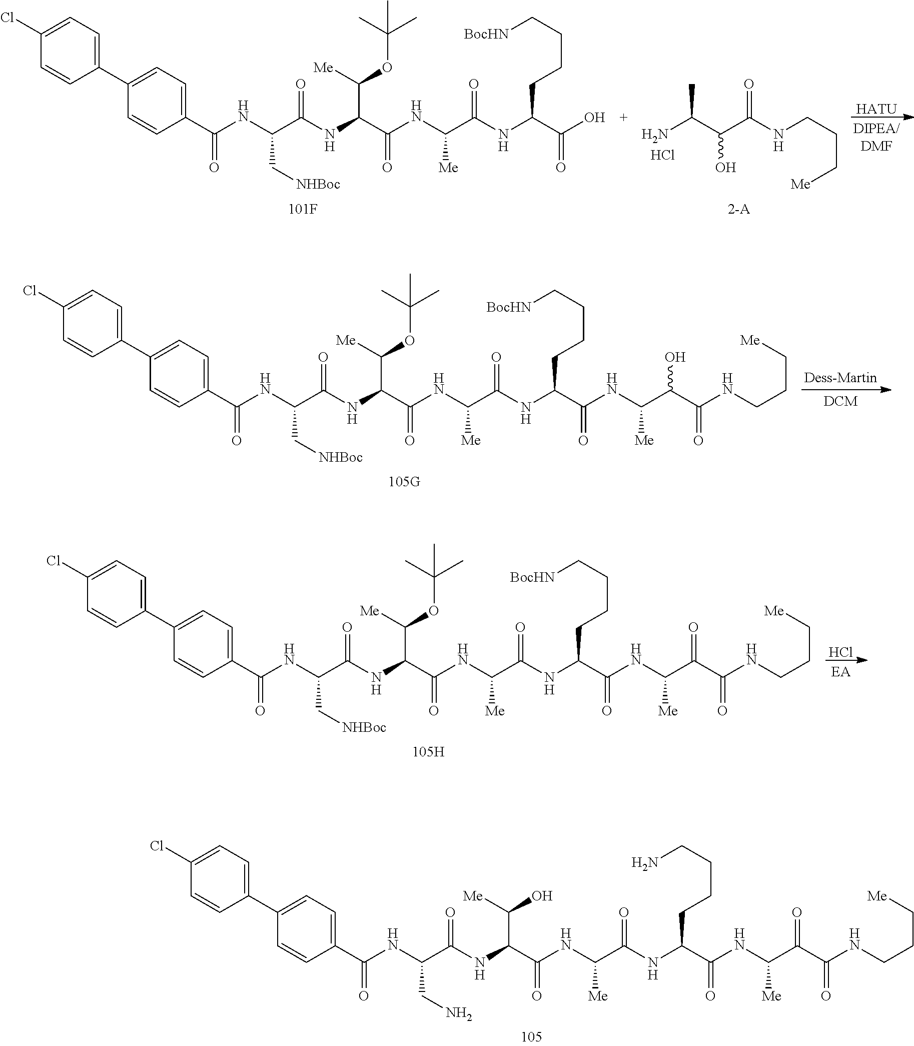

General Method 3.

Peptide coupling of 101F to amino acid 2A, oxidation and sidechain deprotection to give inhibitor 105 is referred to as General Method 3.

Peptide 101F is prepared according to General Method 1. To a solution of peptide 101F (300 mg, 0.337 mmol) in dry DMF (5 mL), compound 2-A (143.2 mg, 0.685 mmol) and DIPEA (221.45 mg, 1.71 mmol) were added in one portion at 0° C. The reaction mixture was stirred at 0° C. for 5 minutes, to which HATU (268.1 mg, 0.685 mmol) was added. The resulting reaction mixture was warmed to room temperature and stirred overnight. The reaction mixture was poured into ice-water (20 mL) and filtered to obtain the crude product which was further purified by silica gel chromatography (eluting with 5% methanol in dichloromethane) to give compound 105G (280 mg, 78.4% yield) as a white solid.

To a solution of compound 105G (280 mg, 0.271 mmol) in anhydrous dichloromethane (10 mL), Dess-Martin reagent (230.25 mg, 0.542 mmol) was added at 0° C. The reaction mixture was stirred at 0° C., slowly warmed up to room temperature and stirred overnight. The reaction mixture was diluted with ethyl acetate (100 mL), washed with NaOH (1M, 10 mL), brine (10 mL) and dried over Na₂SO₄. Crude product was obtained after filtration and concentration, which was further purified by silica gel chromatography (eluting with 5% methanol in dichloromethane) to give compound 105H (156 mg, 56% yield) as a white solid.

Compound 105H (156 mg, 0.15 mmol) was dissolved in HCl/EtOAc (4M, 3 mL). The reaction mixture was stirred at room temperature for 3 h at room temperature and concentrated under reduced pressure to obtain the crude product, which was purified by prep-HPLC to give compound 105 (28.3 mg, 19.1% yield) as a white solid. MS (ESI) for (C₃₇H₅₃ClN₈O₈): m/z 773.4 (M+H). ¹H NMR (400 MHz, CD₃OD), δ 0.91-0.94 (m, 3H), 1.19 (d, J=4.4 Hz, 1H), 1.20 (d, J=4.4 Hz, 1H), 1.20-1.21 (m, 3H), 1.35-1.42 (m, 10H), 1.42-1.44 (m, 3H), 1.67-1.69 (m, 1H), 2.93 (t, J=7.2 Hz, 3H), 3.18-3.24 (m, 2H), 3.24-3.27 (m, 1H), 3.43-3.57 (m, 1H), 4.30-4.48 (m, 5H), 5.04-5.08 (m, 1H), 7.48 (d, J=8.4 Hz, 2H), 7.67 (d, J=8.4 Hz, 2H) 7.76 (d, J=8.4 Hz, 2H), 8.05 (d, J=8.4 Hz, 2H).

### Example 6

### Preparation of Compound 106

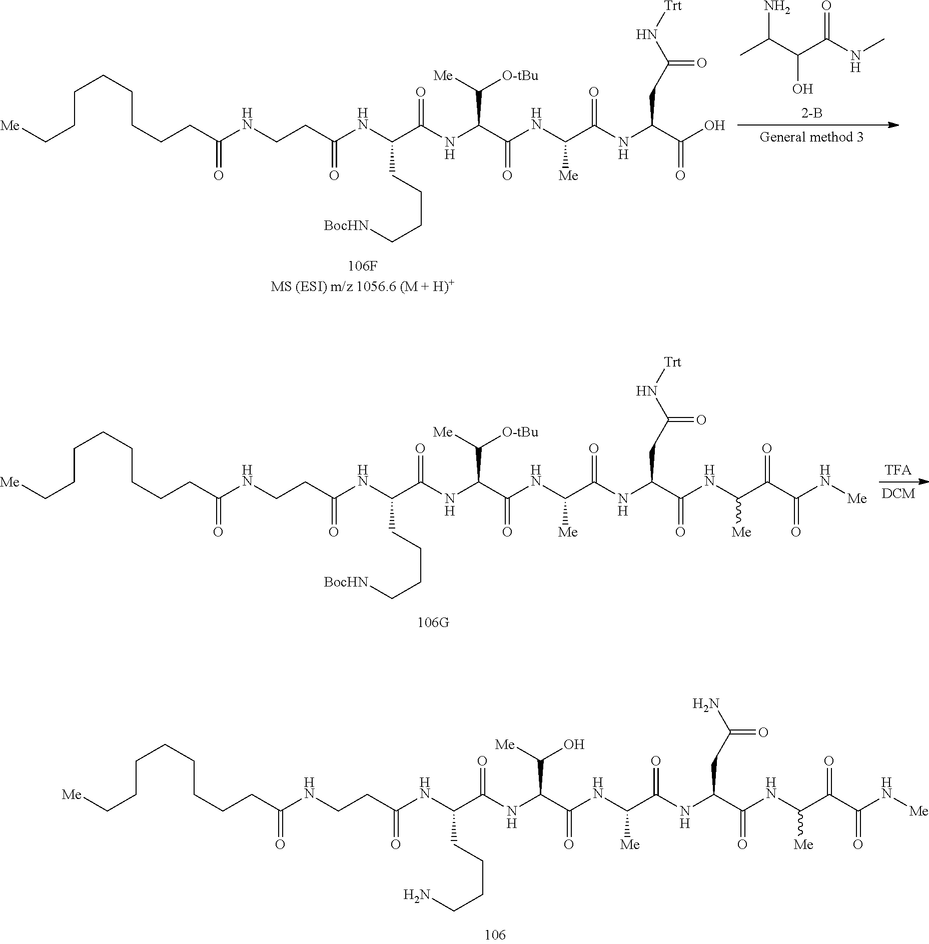

Compound 106F was prepared according to General Method 1 (Example 1).

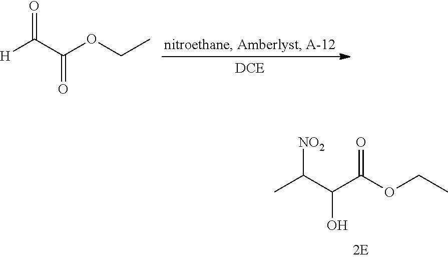

A mixture of nitroethane (3.6 g, 0.5 mol) and Amberlyst A-12 (20 g) in 1,2-dichloroethane (30 mL) was cooled to 0° C. Ethyl glyoxalate (5 g, 50% solution in toluene) was added. The resulting mixture was stirred at room temperature overnight. The mixture was filtered and the filtrate was concentrated in vacuo to give compound 2E (4.2 g, 97% yield), as an oil.

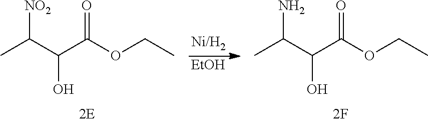

A mixture of 2E (0.2 g, 1.1 mmol) and Raney nickel (0.2 g) in ethanol (5 mL) was subjected to hydrogen gas at 30 psi hydrogen at room temperature for 10 hrs. The mixture was filtered and the filtrate was concentrated in vacuo to afford compound 2F. The residue was used in the next step without further purification.

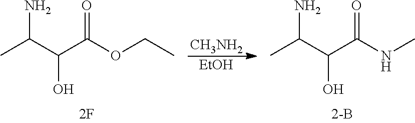

To a 30% solution of methylamine in absolute ethanol (20 mL) was added compound 2F (160 mg, 1 mmol). The solution was refluxed for 2 hrs. After evaporation of the solvent, the residue was recrystallized from dichloromethane/ethyl acetate to give compound 2-B (100 mg, 70% yield), as a yellow solid.

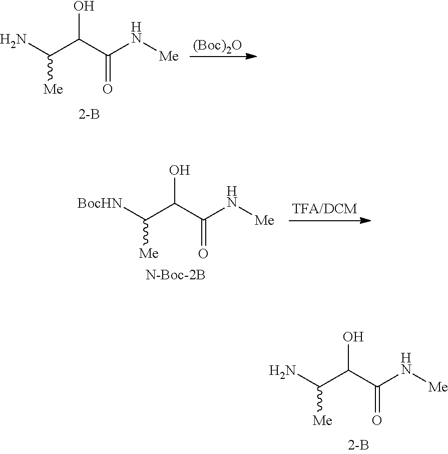

For purification and characterization purposes compound 2-B was protected as an N-Boc derivative, then the Boc group was deprotected. To a solution of compound 2-B (264 mg, 2.0 mmol) in acetone-H₂O (1:1, 6 mL) was added 1M NaOH (6 mL, 3 mmol) and (Boc)₂O (0.69 mL, 3.0 mmol). The mixture was stirred at room temperature for 3 h. Acetone was removed and diluted with H₂O (2 mL). The mixture was extracted with ethyl acetate and the combined organic layers washed with brine, dried over anhydrous Na₂SO₄. After evaporation the residue was purified by ISCO using ethylacetate and hexanes to isolate the N-Boc-2B (120 mg, 26%) as white solid. MS (ESI) m/z 233 (M+H)⁺; t_{R} 2.16 min, (10% CH₃CN/H₂O−90% CH₃CN/H₂O+0.05% TFA), 3 min, 1.0 mL/min, Kinetex C18, 4.6×50 mm).

The Boc group was removed by treatment with 1:4 TFA-DCM and subsequent removal of TFA under reduced pressure to afford compound 2-B and the material was used as such to prepare compounds 106 and 107.

Compound 106 was prepared from peptide 106F and compound 2-B according to the General Method 3 with a modified final deprotection procedure. Compound 106G (32 mg, 0.027 mmol) was dissolved in 20% TFA-DCM (1.0 mL) and the reaction mixture was stirred at rt for 2 h and concentrated under reduced pressure to obtain a brown oily product, which was purified by prep-HPLC (Acetonitrile-water+0.05% TFA) to give compound 106 as a white solid. MS (ESI) m/z 770.3 (M+H)+. t_{R} 5.29 min (10% CH₃CN/H₂O−90% CH₃CN/H₂O+0.05% TFA, 10 min, 0.5 mL/min, Titan C18, 2.1×50 mm).

### Example 7

### Preparation of Compound 107

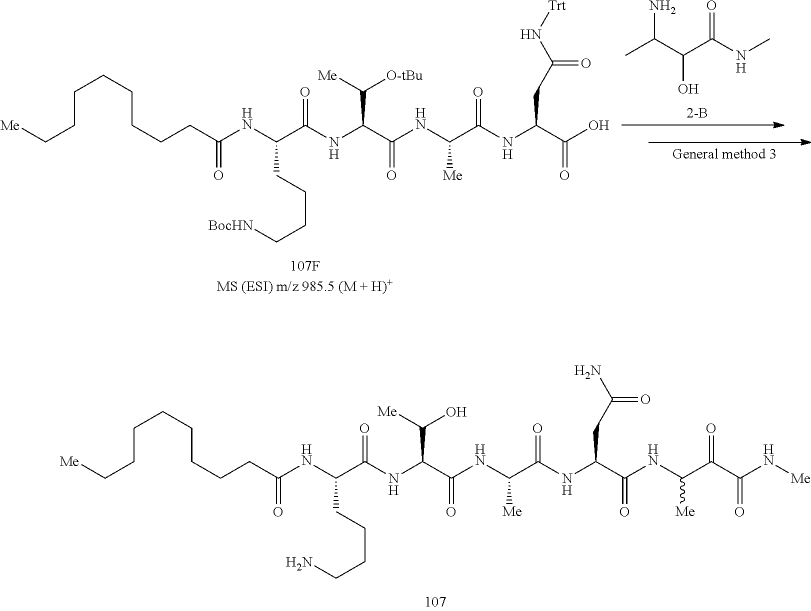

Compound 107F was prepared according to General Method 1 (Example 1). Compound 107 was prepared from peptide 107F according to the General Method 3 and the final deprotection procedure described for compound 107. MS (ESI) m/z 699.2 (M+H)+. t_{R} 4.91 min, (10% CH₃CN/H₂O−90% CH₃CN/H₂O=0.05% TFA, 10 min, 0.5 mL/min, Titan C18, 2.1×50 mm).

### Example 8

### Preparation of Compound 108

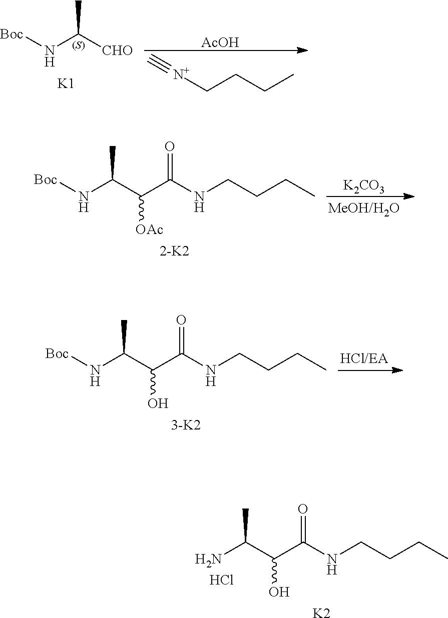

General Method 4.

Isonitrile addition to peptide aldehyde K1, followed by basic, then acidic deprotection to give compound K2 is referred to as General Method 4.

A solution of compound K1 (500 mg, 2.89 mmol) in anhydrous dichloromethane (5 mL), was treated with butyl isonitrile (364.3 mg, 4.33 mmol) and acetic acid (346.7 mg, 5.77 mmol) at 0° C. The reaction mixture was warmed to room temperature and stirred overnight. Solvent was removed under reduce pressure to obtain a crude product which was further purified by silica gel chromatography (eluting with 5% methanol in dichloromethane) to give 2-K2 (595 mg, 75.2% yield) as a white powder.

To a solution of compound 2-K2 (595 mg, 1.88 mmol) in methanol (10 mL) and water (10 mL), K₂CO₃ (2.6 g, 18.8 mmol) was added and the reaction mixture was stirred at room temperature overnight. The reaction mixture was partitioned between EtOAc (100 mL) and water (10 mL). The organic layer was washed with brine, dried over Na₂SO₄, filtrated and concentrated to give a crude product, which was purified by silica gel column chromatography (eluting with 5% methanol in dichloromethane) to give compound 3-K2 (476.7 mg, 92% yield) as a white powder.

A solution of compound 3-K2 (385 mg, 1.40 mmol) in HCl/EtOAc (4M, 5 mL) was stirred at room temperature for 3 h. The mixture was concentrated in vacuum to obtain the crude product K2 which was used in next step without further purification.

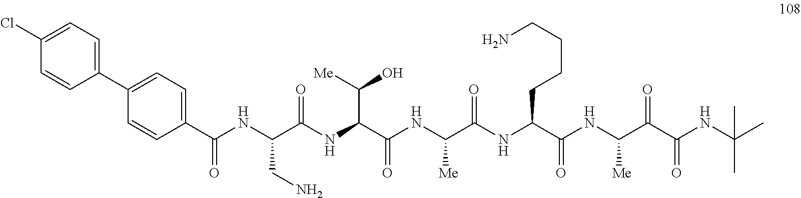 [SMILES: C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)C(=O)NC(C)(C)C]

Compound 108 was prepared according to General Methods 3 and 4, substituting tert-butyl isonitrile for n-butyl isonitrile (40 mg, 31% yield). MS (ESI) for (C₃₇H₅₃ClN₈O₈): m/z 773.1 [M+H]⁺. ¹H NMR (400 MHz, CD₃OD) δ 8.04 (d, J=7.6, 2H), 7.75 (d, J=8.0, 2H), 7.67 (d, J=8.4, 2H), 7.47 (d, J=8.4, 2H), 5.04-5.08 (m, 1H), 4.17-4.37 (m, 5H), 3.57-3.56 (m, 1H), 3.44-3.46 (m, 1H), 2.91-2.94 (m, 2H), 1.64-1.84 (m, 4H), 1.43-1.45 (m, 6H), 1.30-1.35 (m, 10H), 1.19-1.20 (m, 3H), 1.09-1.18 (m, 2H).

### Example 9

### Preparation of Compound 109

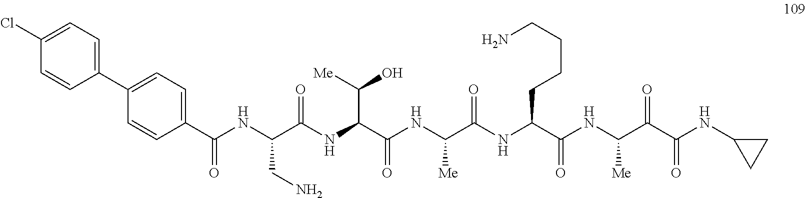 [SMILES: C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)C(=O)NC1CC1]

Compound 109 was prepared according to General Methods 3 and 4, substituting cyclopropyl isonitrile for n-butyl isonitrile. (27.2 mg, 25.2% yield). MS (ESI) for (C₃₆H₄₉ClN₈O₈): m/z 757.3 [M+H]⁺. ¹H NMR (400 MHz, CD₃OD) δ 0.57-0.70 (m, 1H), 0.70-0.72 (m, 2H), 1.10-1.19 (m, 2H), 1.20-1.23 (m, 2H), 1.36-1.40 (m, 6H), 1.42-1.83 (m, 5H), 2.65-2.68 (m, 1H), 2.91-2.95 (m, 2H), 3.40-3.51 (m, 2H), 4.18-4.22 (m, 5H), 7.47 (d, J=8.0 Hz, 2H), 7.66 (d, J=8.4 Hz, 2H), 7.75 (d, J=8.0 Hz, 2H), 8.10 (d, J=8.0 Hz, 2H), 8.48 (s, 2H).

### Example 10

### Preparation of Compound 110

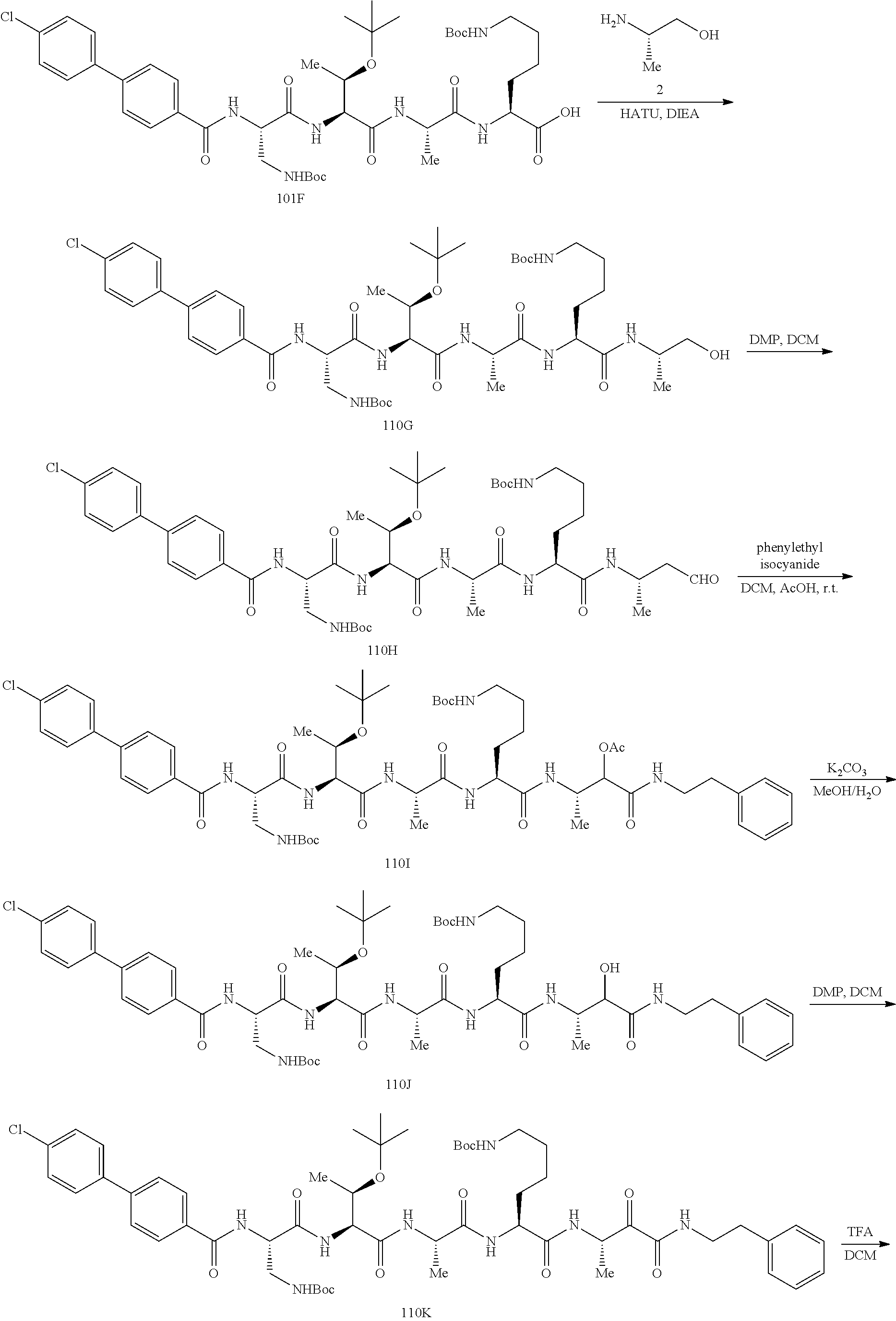

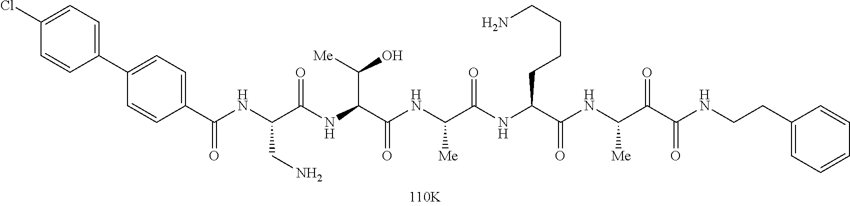 [SMILES: C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)C(=O)NCCc1ccccc1]

General Method 5.

Peptide coupling of 101F to amino alcohol 2, oxidation, isonitrile addition, acetate hydrolysis, oxidation and sidechain deprotection to give compound 110 is referred to as General Method 5.

To a solution a solution of compound 101F (1.0 g, 1.14 mmol) in DMF (10 mL) was added (S)-2-aminopropan-1-ol (171 mg, 2.28 mmol), DIPEA (590 mg, 4.57 mmol) at 0° C. The solution was kept at 0° C. for 10 min, and then HATU (868.6 mg, 2.28 mmol) was added. The reaction mixture was stirred at room temperature for 4 h, then diluted with water (50 mL). The mixture was filtered and the filtrate cake was washed with water (5 mL*3) and PE (5 mL*3) to give compound 110G (852.5 mg, 74.7% yield) used directly in the next step.

To a solution of compound 110G (850 mg, 0.911 mmol) in anhydrous dichloromethane (10 mL), Dess-Martin reagent (773.2 mg, 1.82 mmol) was added at 0° C. The reaction mixture was slowly warmed to room temperature stirred overnight. The reaction mixture was diluted with ethyl acetate (100 mL), washed with NaOH (1M, 10 mL), brine (10 mL), dried over Na₂SO₄ and concentrated to give the crude product, which was further purified by silica gel column chromatography (eluting with 5% methanol in dichloromethane) to give compound 110H (544.3, 64.1% yield) as a white solid.

A solution of compound 110H (400 mg, 0.43 mmol) in anhydrous dichloromethane (5 mL) was treated with phenylethyl isocyanide (132 mg, 1.0 mmol) and acetic acid (51.6 mg, 0.86 mmol) at 0° C. The reaction mixture was warmed to room temperature and stirred overnight. All the volatiles were removed under reduce pressure to give a crude product which was further purified by silica gel column chromatography (eluting with 5% methanol in dichloromethane) to give compound 110I (327 mg, 68% yield) as a white powder.

A solution of compound 110I (327 mg, 0.291 mmol) in methanol and water (10 mL, 1:1) was treated with K₂CO₃ (403 mg, 2.90 mmol) and stirred at room temperature overnight. The reaction mixture was partitioned between EtOAc (100 mL) and water (10 mL), the organic layer was separated and dried over Na₂SO₄. The crude product was obtained after filtration and concentration, which was further purified by silica gel column (eluting with 5% methanol in dichloromethane) to give compound 110J (289 mg, 92% yield) as a white powder.

To a solution of compound 110J (289 mg, 0.267 mmol) in anhydrous dichloromethane (5 mL), Dess-Martin reagent (227 mg, 0.535 mmol) was added at 0° C. The reaction mixture was slowly warmed to room temperature and stirred overnight. The reaction mixture was diluted with ethyl acetate (100 mL), washed with NaOH (1M, 15 mL), brine (20 mL) and dried over Na₂SO₄. Crude product was obtained after filtration and concentration, which was further purified by silica gel column (eluting with 5% methanol in dichloromethane) to give compound 110K. (178.2, 62.1% yield) as a white solid.

To a solution of compound 110K (178 mg, 0.165 mmol) in dichloromethane (3 mL) was added TFA (1 mL). The reaction mixture was stirred at room temperature for 3 hours and concentrated to give the crude product, which was purified by prep-HPLC to give 110 (34.2 mg, 25.1% yield) as a white solid. MS (ESI) for (C₄₁H₅₃ClN₈O₈): m/z 821.3 (M+H). ¹H NMR (400 MHz, CD₃OD) δ 0.96-1.08 (m, 3H), 1.20-1.32 (m, 9H), 1.42-1.84 (m, 4H), 2.76-2.90 (m, 4H), 3.40-3.48 (m, 4H), 4.10-4.44 (m, 6H), 7.19-7.25 (m, 5H), 7.46 (d, J=7.2 Hz, 2H), 7.66 (d, J=7.2 Hz, 2H), 7.70 (d, J=7.6 Hz, 2H), 8.10 (d, J=7.6 Hz, 2H), 8.50 (s, 2H).

### Example 11

### Preparation of Compound 111

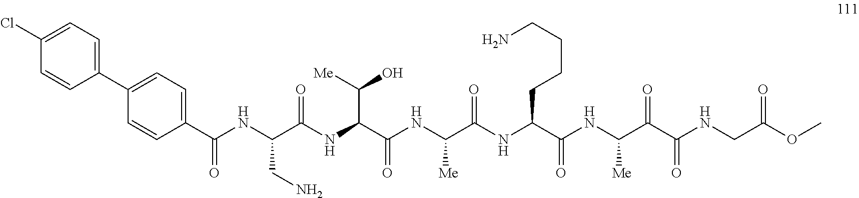 [SMILES: COC(=O)CNC(=O)C(=O)[C@H](C)NC(=O)[C@H](CCCCN)NC(=O)[C@H](C)NC(=O)[C@@H](NC(=O)[C@H](CN)NC(=O)c1ccc(-c2ccc(Cl)cc2)cc1)[C@@H](C)O]

Compound 111 was prepared according to General Method 5, substituting methyl isocyanoacetate for phenylethyl isocyanide. (10 mg, 11% yield). MS (ESI) for (C₃₆H₄₉ClN₈O₁₀): m/z 789.3 [M+H]⁺. ¹H NMR (400 MHz, CD₃OD) δ 1.13-1.31 (m, 6H), 1.40-1.55 (m, 6H), 1.63-1.75 (m, 2H), 1.83-1.95 (m, 1H), 2.95 (m, 2H), 3.74 (s, 3H), 3.98-4.71 (m, 8H), 7.50 (d, J=8 Hz, 2H), 7.70 (d, J=8 Hz, 2H), 7.78 (d, J=8 Hz, 2H), 8.03 (d, J=8 Hz, 2H), 8.54 (s, 1H).

### Example 12

### Preparation of Compound 112

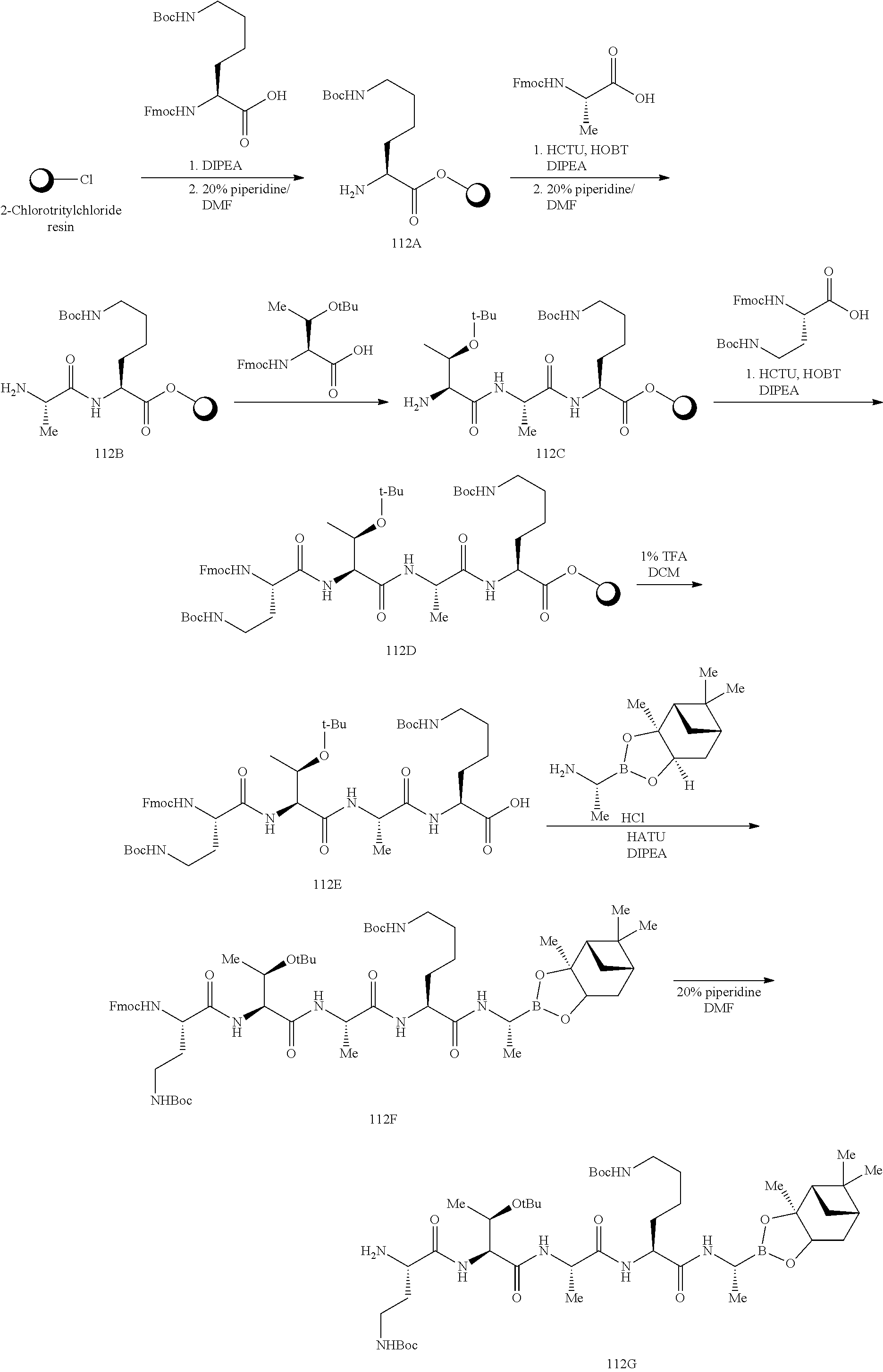

A mixture of 2-chlorotrityl resin (0.320 g, 0.416 mmol), DIPEA (0.215 g, 1.66 mmol) in dry DCM (15.0 mL) was added to a solution of Fmoc-L-Lys(Boc)-OH (0.389 g, 0.832 mmol) in dry DCM (10.0 mL) at 0° C. The mixture was then shaken for 5 hrs at room temperature. The mixture was filtered and the cake was washed with DCM (20.0 mL×3), DMF (20.0 mL×3) MeOH (20.0 mL×3). To the above resin was added 20% piperidine/DMF (approximately 20.0 mL) to remove the Fmoc group. The mixture was shaken for 10 mins and the cycle was repeated three times. The mixture was then washed with DMF (20.0 mL×3 mL) and DCM (20.0 mL×3) to give compound 112A.

To a mixture of Fmoc-L-Ala-OH (0.259 g, 0.832 mmol) in dry DMF (15.0 mL) was added HCTU (0.344 g, 0.832 mmol), HOBt (0.112 g, 0.832 mmol), DIEA (0.215 g, 1.66 mmol) at 0° C. The mixture was then was stirred at 16° C. for 30 mins. The mixture was added to a suspension of Compound 112A (0.416 mmol) in DMF (10.0 mL). The mixture was stirred at room temperature for 1.5 hrs. After ELSD showed the reaction was completed, the mixture was filtered. The cake was washed with DMF (20.0 mL×3) and DCM (20.0 mL×3). To the above resin was added approximately 20.0 mL 20% piperdine/DMF to remove the Fmoc group. The mixture was shaken for 10 mins and the cycle was repeated three times. The mixture was then washed with DCM (20.0 mL×3 mL) and DMF (20.0 mL×3) to give compound 112B.

Compound 112C was made using the same method as for compound 112B except Fmoc-L-Thr(tBu)-OH was utilized in the coupling reaction in place of Fmoc-L-Ala-OH.

Compound 112D was made from Compound 112C using the same method as for compound 112C except Fmoc-L-Dab(Boc)-OH was utilized in the coupling reaction in place of Fmoc-L-Thr(tBu)-OH.

A mixture of Compound 112D (2.00 mmol) in TFA/DCM (1%, 20.0 mL) was shaken at 15° C. for 10 mins. The mixture was then filtered and the filtrate was treated saturated NaHCO₃ solution until pH=7˜8. The mixture was treated with DCM (20.0 mL). The aqueous layer was added citric acid until pH ˜3-4. The mixture was extracted with DCM (20.0 mL×3). The combined organic layers were washed with brine, dried over Na₂SO₄ and concentrated to give compound 112E (1.1 g, 61.5%). MS (ESI) m/z 919.3 (M+Na)⁺.

Compound 112E (250 mg, 0.279 mmol), HATU (212 mg, 0.558 mmol) and (R)-BoroAla-(+)-Pinanediol-HCl (108 mg, 0.419 mmol) were placed in the flask in an ice bath, then DCM (2.40 mL) and DMF (0.800 mL) were added. DIEA (108 mg, 0.837 mmol) was then added to the mixture. The reaction mixture was stirred at −5° C. for 30 mins. The crude residue was taken up in DMSO. A second experiment starting from 250 mg of Compound 112E was repeated and combined with this experiment. The combined batches were purified by prep-HPLC to give compound 112F (200 mg, 81.4%) as white solid. MS (ESI) m/z 1102.4 (M+H)⁺.

To a solution of Compound 112F (400 mg, 0.363 mmol) in MeCN (3 ml) was added Et₂NH (79.6 mg, 1.09 mmol). The mixture was then stirred at 16° C. for 12 hrs until TLC (DCM:MeOH 10:1, R_{f}=0.5) showed the reaction was complete. The mixture was concentrated and the residue was purified by column chromatography to give compound 112G (280 mg, 87.8%). MS (ESI) m/z 880.6 (M+Na)⁺.

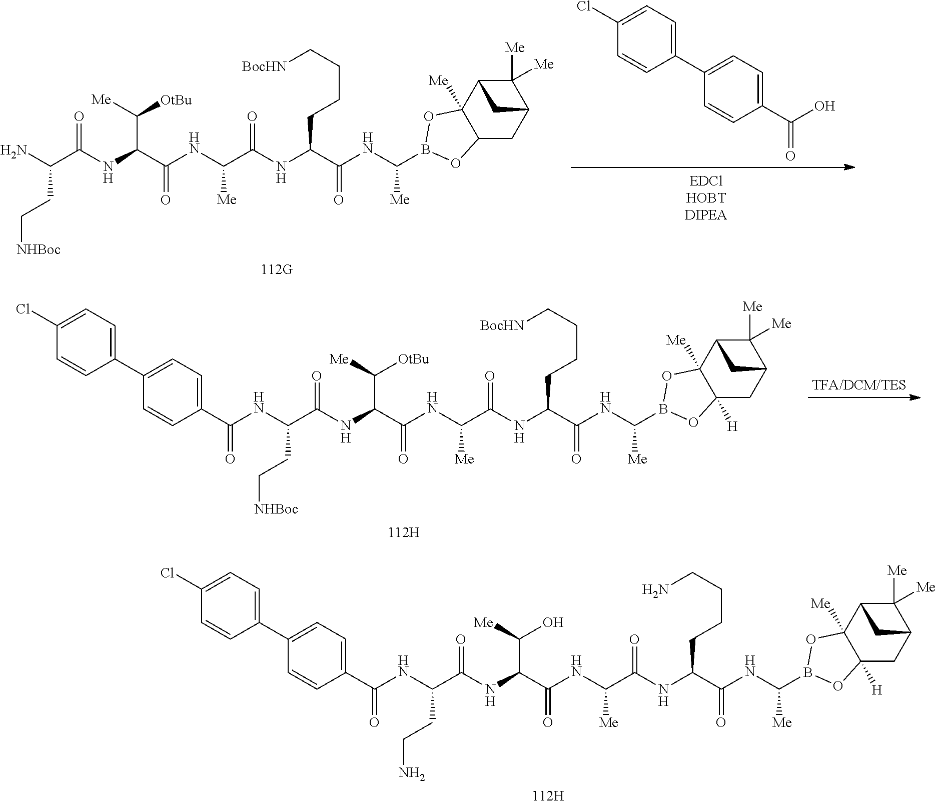

General Method 6:

Coupling of compound 112G with a carboxylic acid in solution phase followed by deprotection of acid sensitive protecting groups with TFA. A specific example is shown to illustrate this method.

To a mixture of compound 112G (60 mg, 0.068 mmol), 4-(4-chlorophenyl)benzoic acid (17.3 mg, 0.0683 mmol), EDCI (26.2 mg, 0.137 mmol), HOBt (18.4 mg, 0.137 mmol) in DMF (2.00 mL) was added DIEA (17.6 mg, 0.137 mmol). The mixture was then stirred at room temperature for 12 hrs. When TLC analysis (DCM:MeOH 10:1, R_{f}=0.5) showed the reaction was complete, the mixture was diluted with water, filtered and the filter cake was washed with water, dried to afford compound 112H (50 mg, yield: 63.3%) as brown solid.

A solution of compound 112H (50.0 mg, 0.0448 mmol) in TFA: DCM: TES (50:45:5) (2.00 mL) was stirred at 12° C. for 0.5 h, then TFA was removed and ELSD showed the reaction was complete. The crude residue was taken up in DMSO and purified by prep-HPLC to give compound 112 (6.3 mg, 16.4%) as an off-white solid. MS (ESI) m/z 838.3 (M+H)⁺. t_{R} 1.37 min (30% CH₃CN/H20−90% CH₃CN/H20.4 min, 0.8 mL/min Luna C18, 2×50 mm).

### Example 13

### Preparation of Compound 113

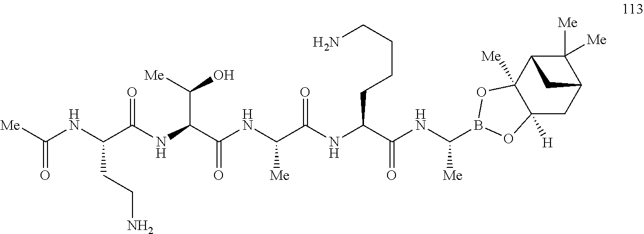 [SMILES: CC(=O)N[C@@H](CCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 113 was prepared according to General Method 6 from 112G and acetic acid. MS (ESI) m/z 666.2 (M+H)⁺. t_{R} 2.27 min (10% CH₃CN/H20−80% CH₃CN/H2O, 4 min, 0.8 mL/min Luna C18, 2×50 mm).

### Example 14

### Preparation of Compound 114

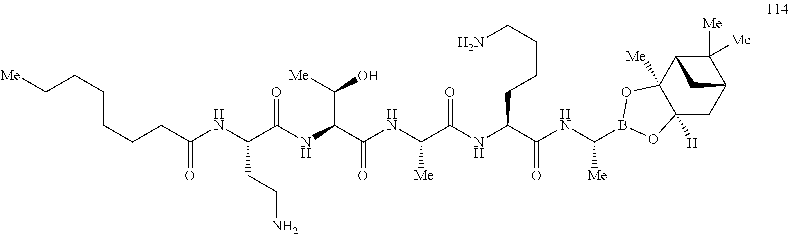 [SMILES: CCCCCCCC(=O)N[C@@H](CCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 114 was prepared according to General Method 6 from 112G and octanoic acid. MS (ESI) m/z 750.5 (M+H)⁺. t_{R} 2.27 min (10% CH₃CN/H20−80% CH₃CN/H2O, 4 min, 0.8 mL/min Luna C18, 2×50 mm).

### Example 15

### Preparation of Compound 115

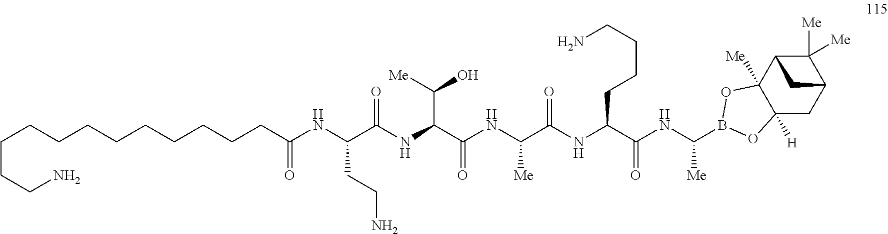 [SMILES: C[C@H](NC(=O)[C@H](CCCCN)NC(=O)[C@H](C)NC(=O)[C@@H](NC(=O)[C@H](CCN)NC(=O)CCCCCCCCCCCCN)[C@@H](C)O)B1O[C@@H]2C[C@@H]3C[C@@H](C3(C)C)[C@]2(C)O1]

Compound 115 was prepared according to General Method 6 from 112G and 13-((tert-butoxycarbonyl)amino)tridecanoic acid. MS (ESI) m/z 835.6 (M+H)⁺. t_{R} 1.22 min (10% CH₃CN/H₂O−80% CH₃CN/H₂O, 1.5 min, 1 mL/min Luna C18, 2×30 mm).

### Example 16

### Preparation of Compound 116

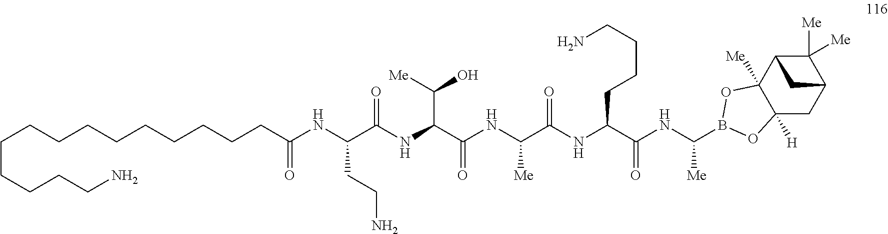 [SMILES: C[C@H](NC(=O)[C@H](CCCCN)NC(=O)[C@H](C)NC(=O)[C@@H](NC(=O)[C@H](CCN)NC(=O)CCCCCCCCCCCCCCN)[C@@H](C)O)B1O[C@@H]2C[C@@H]3C[C@@H](C3(C)C)[C@]2(C)O1]

Compound 116 was prepared according to General Method 6 from 112G and 15-((tert-butoxycarbonyl)amino)pentadecanoic acid. MS (ESI) m/z 863.6 (M+H)⁺. t_{R} 1.79 min (10% CH₃CN/H₂O, 0.3 min; 10%−80% CH₃CN/H₂O, 1.1 min, 1 mL/min Luna C18, 2×30 mm).

### Example 17

### Preparation of Compound 117

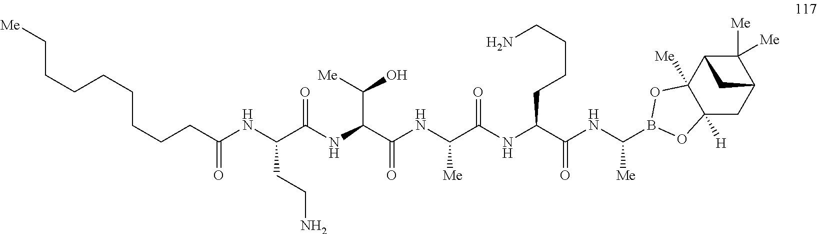 [SMILES: CCCCCCCCCC(=O)N[C@@H](CCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 117 was prepared according to General Method 6 from 112G and decanoic acid. MS (ESI) m/z 778.5 (M+H)⁺.

### Example 18

### Preparation of Compound 118

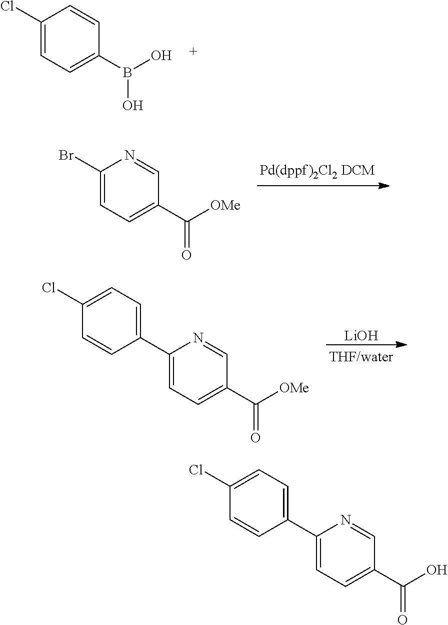

In a sealed tube apparatus with a screw-cap vial, 4-chlorophenylboronic acid (0.69 g, 4.4 mmol), methyl 6-bromonicotinate (0.85 g, 4.0 mmol), Pd(dppf)₂Cl₂ DCM (98 mg, 0.12 mmol) were added. The flask was flushed with nitrogen, then 20 mL THF (bubbled with N₂ for 3 min) was added and the mixture was heated at 90° C. for 20 hr. The mixture was portioned between EtOAc and saturated NH₄Cl, and the aqueous layer was extracted with EtOAc. The organic layers were washed with brine, dried over Na₂SO₄, filtered, and concentrated. Flash chromatography (100% DCM to 4% MeOH/DCM) afforded 0.72 g (72%) of methyl 6-(4-chlorophenyl)nicotinate. MS (ESI) m/z 248.1 (M+H)⁺.

A mixture of 6-(4-chlorophenyl)nicotinate (99 mg, 0.40 mmol), LiOH (21 mg, 0.88 mmol) in 3 mL THF and 1 mL water was stirred at rt for 2 hr. To this mixture was added 0.2 M NaHSO4 (4.4 mL) slowly. A precipitate was formed, and the solution was cooled in an ice bath. The solid was filtered, and washed three times with water, then rinsed with ether to afford 61 mg (65%) of 6-(4-chlorophenyl)nicotinic acid, an off-white solid. MS (ESI) m/z 234.0 (M+H)⁺.

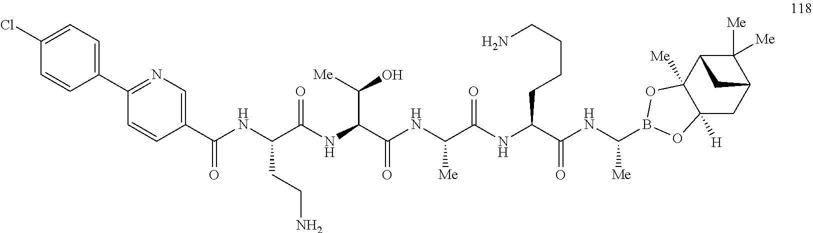 [SMILES: C[C@H](NC(=O)[C@H](CCCCN)NC(=O)[C@H](C)NC(=O)[C@@H](NC(=O)[C@H](CCN)NC(=O)c1ccc(-c2ccc(Cl)cc2)nc1)[C@@H](C)O)B1O[C@@H]2C[C@@H]3C[C@@H](C3(C)C)[C@]2(C)O1]

Compound 118 was prepared according to General Method 6 from 112G and 6-(4-chlorophenyl)nicotinic acid. In this example, the purification was performed using reverse-phase chromatography (C18, 5% CH₃CN/H₂O to 100% CH₃CN with 0.1% TFA) to afford Compound 118 as the TFA salt. MS (ESI) m/z 839.5 (M+H)⁺; t_{R} 2.73 min (10% CH₃CN/H₂O−90% CH₃CN/H₂O+0.05% TFA, 4 min, 1.0 mL/min Titan C18, 2.1×50 mm).

### Example 19

### Preparation of Compound 119

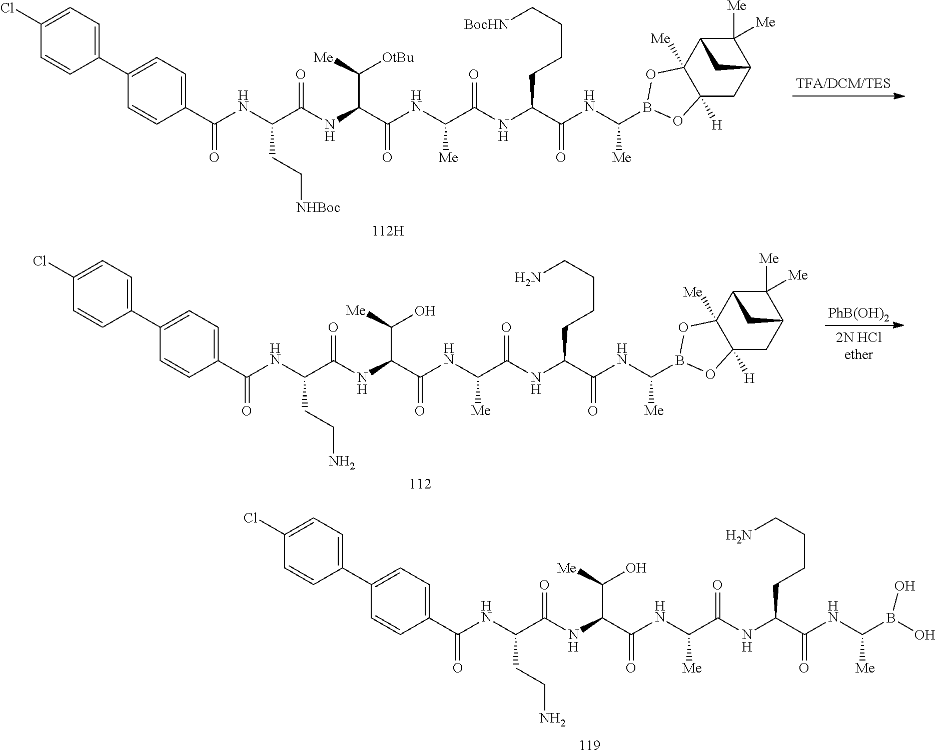 [SMILES: C[C@H](NC(=O)[C@H](CCCCNC(=O)OC(C)(C)C)NC(=O)[C@H](C)NC(=O)[C@@H](NC(=O)[C@H](CCNC(=O)OC(C)(C)C)NC(=O)c1ccc(-c2ccc(Cl)cc2)cc1)[C@@H](C)OC(C)(C)C)B1O[C@@H]2C[C@@H]3C[C@@H](C3(C)C)[C@]2(C)O1 ; "PhB(OH)2" ; C[C@H](NC(=O)[C@H](CCCCN)NC(=O)[C@H](C)NC(=O)[C@@H](NC(=O)[C@H](CCN)NC(=O)c1ccc(-c2ccc(Cl)cc2)cc1)[C@@H](C)O)B1O[C@@H]2C[C@@H]3C[C@@H](C3(C)C)[C@]2(C)O1 ; C[C@H](NC(=O)[C@H](CCCCN)NC(=O)[C@H](C)NC(=O)[C@@H](NC(=O)[C@H](CCN)NC(=O)c1ccc(-c2ccc(Cl)cc2)cc1)[C@@H](C)O)B(O)O]

A mixture of compound 112H (123 mg, 0.11 mmol) and triethylsilane (26 mg, 0.22 mmol) in 3 mL DCM was cooled to 0° C., then 0.75 mL TFA was added dropwise. After 1 hr, the solution was concentrated under reduced pressure to afford an oil. The oil was dissolved in 8 mL water and a solution of PhB(OH)₂ (41 mg, 0.34 mmol) in 6 mL water followed by 2N HCl (0.28 mL) and 14 mL ether was added, and the mixture was stirred vigorously for 1.5 hr. The mixture was treated with 2 mL hexanes to separate the emulsion, and the organic layer was drawn off. The aqueous layer was extracted with 1:1 ether:hexanes (2×). The aqueous layer was lyophilized to a solid, and was subjected to purification by reverse phase column chromatography (C18, 5% CH₃CN/H₂O to 100% CH₃CN in 0.005M HCl) to afford 11 mg (14%) of compound 119, a white solid, as the bis-HCl salt. MS (ESI) m/z 726.5 (M+Na)⁺; t_{R} 3.21 min (10% CH₃CN/H₂O−95% CH₃CN/H₂O, 6 min, 1 mL/min Gemini-NX C18, 4.6×50 mm).

### Example 20

### Preparation of Compound 120

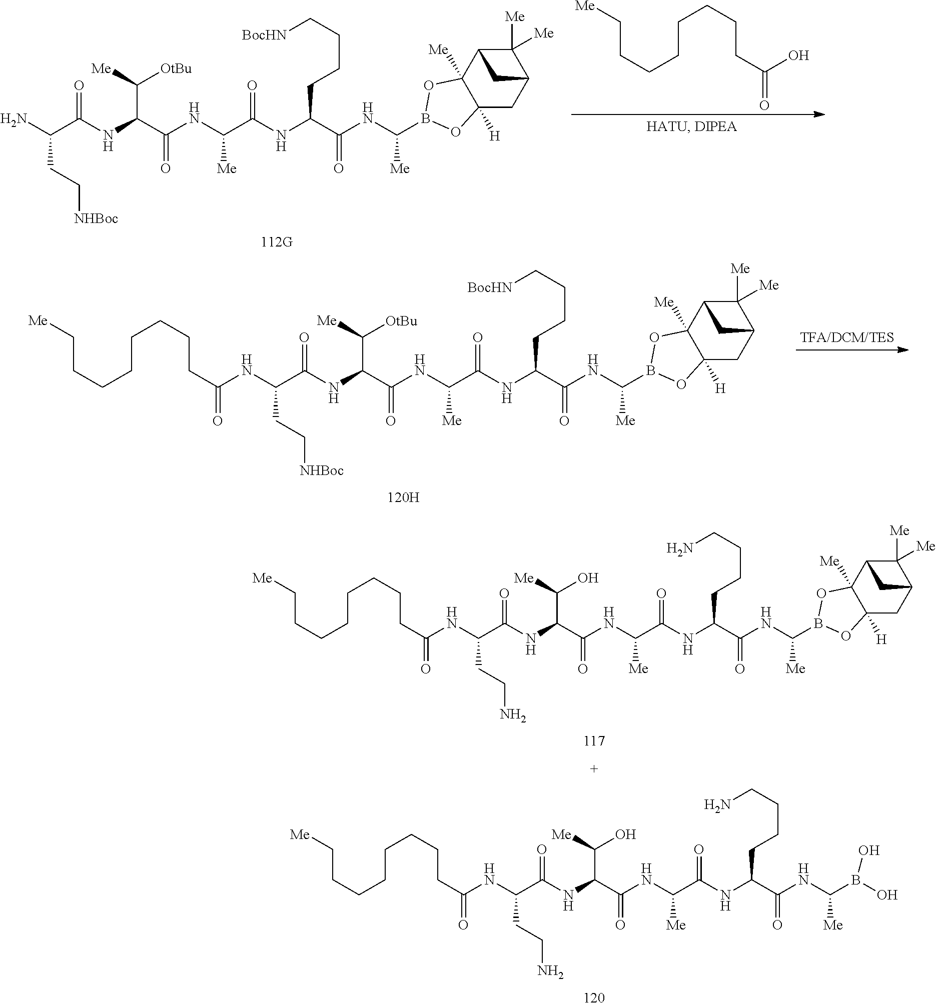

A solution of compound 112G (0.35 g, 0.40 mmol), decanoic acid (76 mg, 0.44 mmol) and HATU (0.18 g, 0.48 mmol) in 4 mL DMF and 4 mL DCM was cooled to 0° C. DIPEA (0.11 g, 0.88 mmol) was added, and the mixture was allowed to warm to rt and was stirred at rt for 1 hr. The mixture was partitioned between DCM and water, and the aqueous layer was extracted with DCM. The combined organic layers were washed sequentially with 0.1 N NaHSO₄ and saturated NaHCO₃. The DCM was evaporated under reduced pressure, and the residue was partitioned between EtOAc and water. A small amount of DCM was added to aid the separation of the emulsion. The organic layer was washed with brine, dried over Na₂SO₄, filtered and concentrated. Flash chromatography (1% MeOH/DCM to 12% MeOH/DCM) afforded 0.18 g (52%) of compound 120H.

A solution of compound 120H (25 mg, 0.024 mmol) and triethylsilane (5.8 mg, 0.05 mmol) in DCM was cooled to 0° C. and 0.2 mL TFA was added dropwise. The solution was allowed to warm to rt and was stirred for 1.5 hr (confirmed consumption of starting material by LC-MS). The solvents were removed under reduced pressure. Reverse-phase chromatography (C18, 5% CH₃CN/H₂O to 100% CH₃CN plus 0.005M HCl) afforded 7.1 mg (35%) of compound 117, and 5.3 mg (31%) of compound 120, in which the pinanediol group was removed during the chromatography. Data for compound 120: MS (ESI) m/z 626 (M−H₂O+H)⁺; t_{R} 3.29 min (10% CH₃CN/H₂O−95% CH₃CN/H₂O, 6 min, 1 mL/min, Gemini-NX C18, 4.6×50 mm).

### Example 21

### Preparation of Compound 121

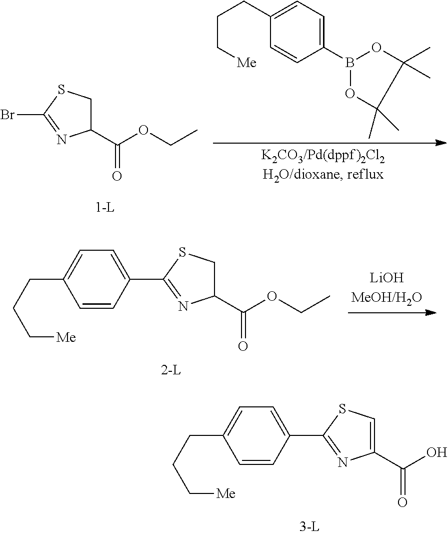 [SMILES: CCOC(=O)C1CSC(Br)=N1 ; CCCCc1ccc(B2OC(C)(C)C(C)(C)O2)cc1 ; CCCCc1ccc(C2=NC(C(=O)OCC)CS2)cc1 ; CCCCc1ccc(-c2nc(C(=O)O)cs2)cc1]

General Method 7:

The synthesis of a biaryl or aryl-heteroaryl carboxylic acid from 4-butylbenzeneboronic acid or 4-butylbenzeneboronic acid pinacol ester and an aryl- or heteroaryl halide. An illustration of this method is depicted for Compound 2-L. A solution of 2-(4-butylphenyl)-4,4,5,5-tetramethyl-1,3,2-dioxaborolane (2.2 g, 8.4 mmol) in dioxane/H₂O (80 mL, v/v, 1/1) was added ethyl 2-bromothiazole-4-carboxylate (1-L) (1.0 g, 4.2 mmol), K₂CO₃ (1.16, 8.4 mmol) and Pd(dppf)Cl₂ (0.31 g, 0.42 mmol) and the flask was flushed with N₂. The mixture was heated to reflux for 7 hrs. After TLC showed the reaction was completed, the dioxane was concentrated under reduced pressure. The residue was adjusted pH=4˜5 with 1 N HCl solution. The resulting mixture was filtered and the filter cake was washed with water, dried to give 0.7 g (58%) of compound 2-L.

Compound 2-L was dissolved in MeOH and water (1:1) and LiOH (0.18 g, 7.3 mmol) was added and the mixture was stirred at room temperature. After TLC showed the reaction was completed, the MeOH was removed. The residue was adjusted pH=4˜5 with 1 N HCl solution. The resulting mixture was filtered and the cake was washed with water, dried to give 0.39 g of 3-L as a brown solid. MS (ESI) m/z 261.8 (M+H)⁺.

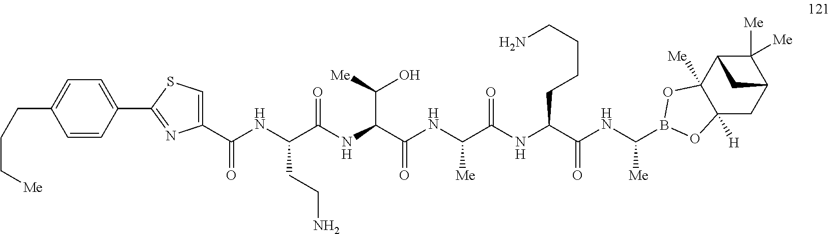 [SMILES: CCCCc1ccc(-c2nc(C(=O)N[C@@H](CCN)C(=O)N[C@H](C(=O)N[C@@H](C)C(=O)N[C@@H](CCCCN)C(=O)N[C@@H](C)B3O[C@@H]4C[C@@H]5C[C@@H](C5(C)C)[C@]4(C)O3)[C@@H](C)O)cs2)cc1]

Compound 121 was prepared according to General Method 6 from 112G and 3-L. MS (ESI) m/z 867.5 (M+H)⁺. t_{R} 3.32 min (10% CH₃CN/H₂O−80% CH₃CN/H₂O, 4 min, 3 mL/min Venusil MP C18, 4.6×50 mm).

### Example 22

### Preparation of Compound 122

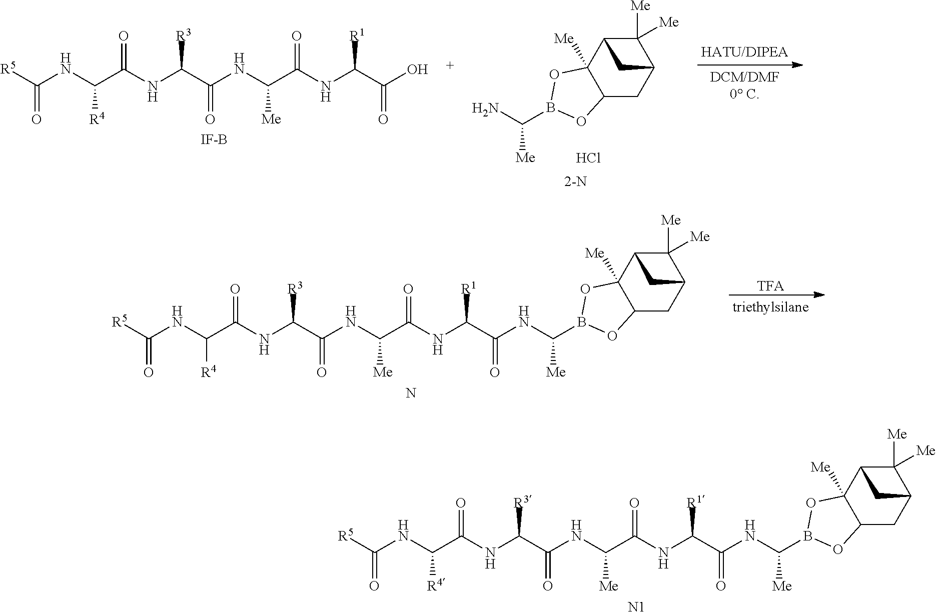

General Method 8:

The coupling of an aminoboronate ester to a carboxylic acid followed by global acid deprotection. Compound 1F-B (1 eq), HATU (2.0 eq) and (R)-BoroAla-(+)-pinanediol HCl (2-N) (1.5 eq) was added to a round-bottom flask and cooled in an ice bath. DCM and DMF were added in a 3:1 ratio (0.03-0.05 M). In cases where solubility is limiting, additional DMF can be added. DIPEA (3 eq) was then added dropwise. After 15-30 minutes, the reaction was allowed to warm to room temperature and stirred for 30 minutes. After LCMS analysis showed the reaction to be complete, the mixture was distributed between DCM and water, and the aqueous layer was extracted twice with DCM. The combined organic layers were washed sequentially with diluted HCl (<0.1 M), NaHCO₃ solution, and brine. The solvent was removed under reduced pressure. The solid residue was washed with acetonitrile to afford the desired compound. In cases where there is excessive DMF remaining, the residue was distributed between EtOAc (300 mL/mmol) mL): water (100 mL/mmol). The organic layers were washed sequentially with water and brine, and dried over Na₂SO₄. The mixture was filtered and concentrated, and the resulting solid washed with acetonitrile to give compound N.

Deprotection: The deprotection of acid sensitive protecting groups (N-Boc, 04-butyl, and/or C(O)NH-trityl) with TFA and triethylsilane. A solution of the fully protected compound N (100 mg, 0.070-0.12 mmol) in TFA: DCM: TES (50:45:5) (1 mL) was stirred at room temperature for 30 min. When analysis by LC-MS showed the reaction was complete, the TFA was evaporated and ELSD showed the reaction was complete. The crude residue was then taken up in DMSO and purified by prep-HPLC to afford Compound N1. In cases where the mobile phase was acetonitrile/water with 0.1% TFA, the resultant salt is the TFA salt. In instances where the mobile phase was acetonitrile/water with 0.1% HCl, the resultant salt is the HCl salt.

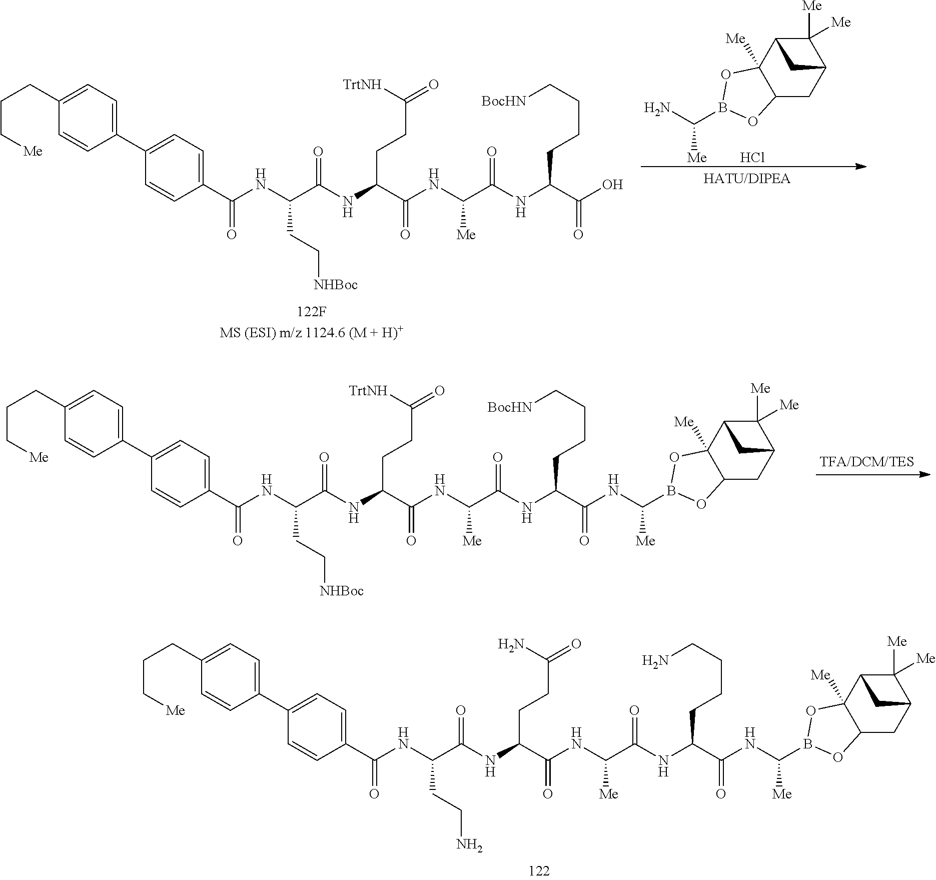

Compound 122 was prepared using General Methods 1 and 8 from compound 122F CH₃CN/H₂O−80% CH₃CN/H₂O, 4 min, 0.8 mL/min Luna C18, 2×50 mm).

### Example 23

### Preparation of Compound 123

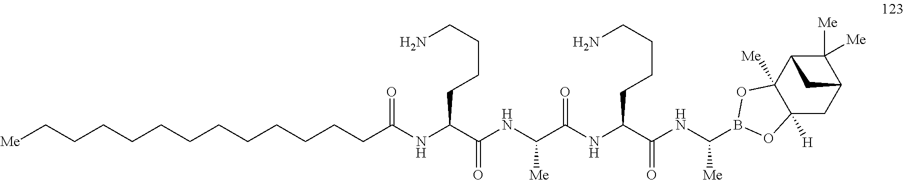 [SMILES: CCCCCCCCCCCCCC(=O)N[C@@H](CCCCN)C(=O)N[C@@H](C)C(=O)N[C@@H](CCCCN)C(=O)N[C@@H](C)B1O[C@@H]2C[C@@H]3C[C@@H](C3(C)C)[C@]2(C)O1]

Compound 123 was prepared using General Methods 1 and 8. MS (ESI) m/z 761.4 (M+H)⁺; t_{R} 3.11 min (10% CH₃CN/H₂O−80% CH₃CN/H₂O, 4 min, 0.8 mL/min Luna C18, 2×50 mm).

### Example 24

### Preparation of Compound 124

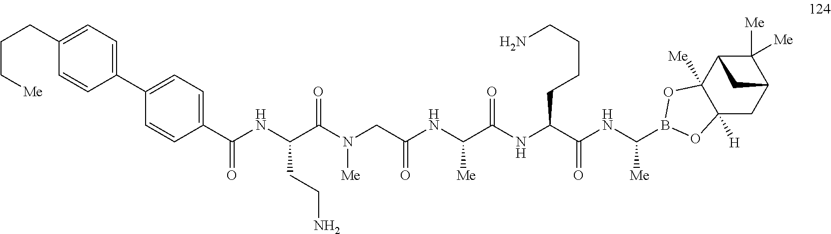 [SMILES: CCCCc1ccc(-c2ccc(C(=O)N[C@@H](CCN)C(=O)N(C)CC(=O)N[C@@H](C)C(=O)N[C@@H](CCCCN)C(=O)N[C@@H](C)B3O[C@@H]4C[C@@H]5C[C@@H](C5(C)C)[C@]4(C)O3)cc2)cc1]

Compound 124 was prepared using General Methods 1 and 8. MS (ESI) m/z 830.5 (M+H)⁺; t_{R} 1.89 min (30% CH₃CN/H₂O−90% CH₃CN/H₂O, 4 min, 0.8 mL/min Luna C18, 2×50 mm).

### Example 25

### Preparation of Compound 125

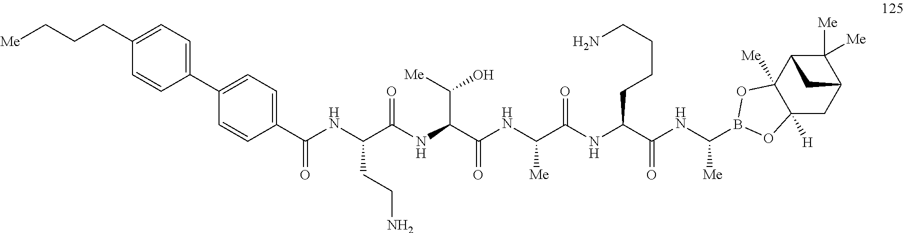 [SMILES: CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)B3O[C@@H]4C[C@@H]5C[C@@H](C5(C)C)[C@]4(C)O3)[C@H](C)O)cc2)cc1]

Compound 125 was prepared using General Methods 1 and 8. MS (ESI) m/z 860.6 (M+H)⁺; t_{R} 2.86 min (10% CH₃CN/H₂O−90% CH₃CN/H₂O, 4 min, 0.8 mL/min Luna C18, 2×50 mm).

### Example 26

### Preparation of Compound 126

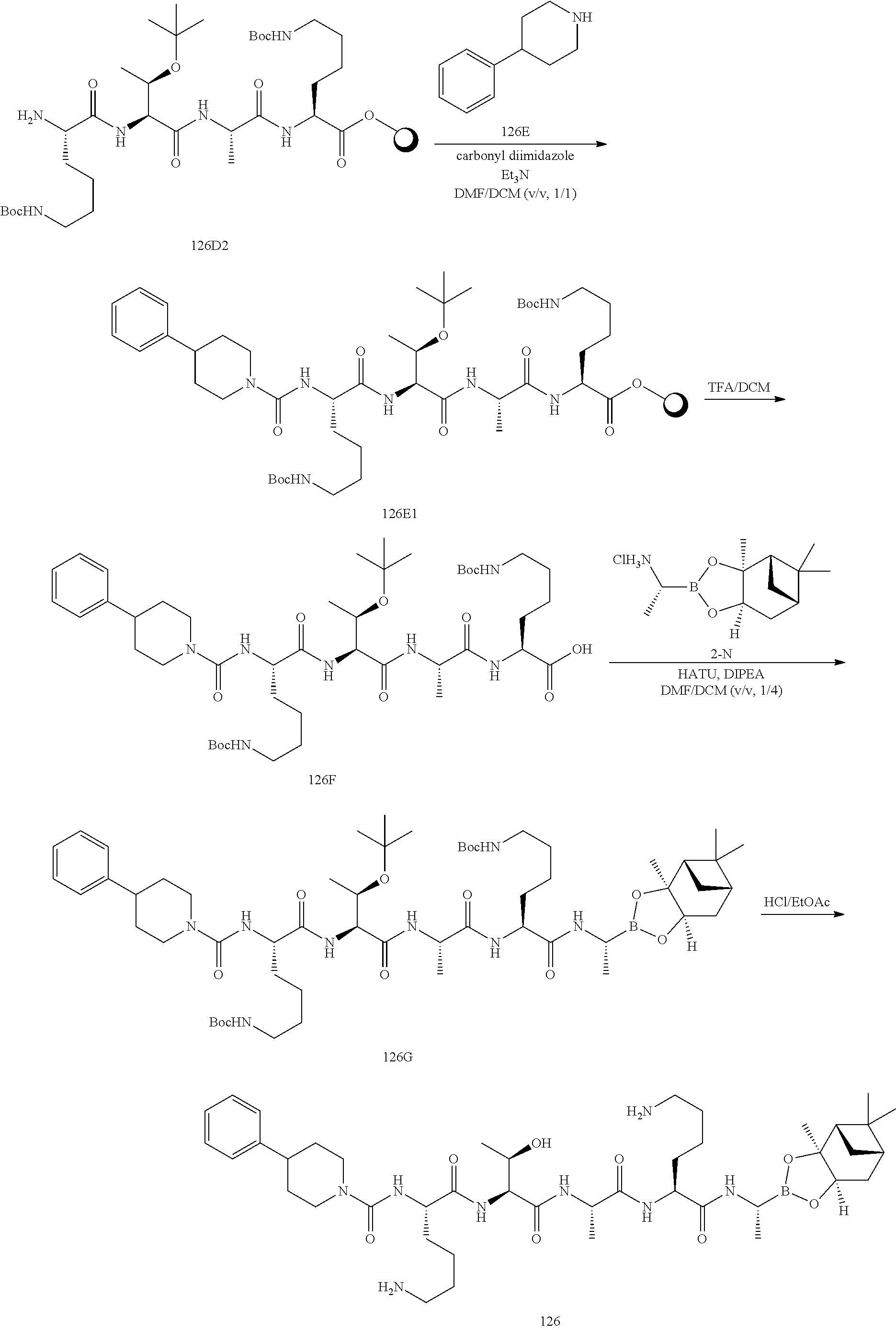

Peptide 126D2 is prepared according to General Method 1. A mixture of CDI (1 g, 12.4 mmol) and peptide 126D2 (10 g, 5.6 mmol) in DMF (100 mL) was stirred at 0° C. for 1 h, then Et₃N (1.26 g, 12.4 mmol) and compound 126E (2 g, 6.2 mmol) were added at 0° C. The reaction mixture was stirred at room temperature for 16 h. The reaction mixture was filtered and the solid was washed with DMF/DCM (50 mL×2), then CH₃OH (20 mL) to give compound 126E1 (10 g, 83%).

A mixture of compound 126E1 (10 g, 4.6 mmol) in HOAc (10 mL)/TFE (10 mL)/DCM (80 mL) was stirred at room temperature for 5 h. The reaction mixture was filtered, the filtrate was concentrated, and the residue was washed with DCM (5 mL)/PE (50 mL) to give compound 126F (1.75 g, 21.8%).

To a mixture of compound 126F (1.75 g, 1.97 mmol) in DMF (4 mL)/DCM (16 mL) was added HATU (1.50 g, 3.94 mmol), (R)-BoroAla-(+)-Pinanediol HCl (1.02 g, 3.94 mmol) in an ice bath. DIPEA (0.76 g, 5.9 mmol) in DMF (2.5 mL)/DCM (2.5 mL) was added dropwise after 10 min, and the reaction was stirred for 1.5 h at 0° C. When the starting material was consumed, the reaction mixture was concentrated and the residue was poured into H₂O (20 mL), extracted by EtOAc (20 mL×3), dried with Na₂SO₄ and concentrated. The crude product was purified by prep-HPLC (CH₃CN/H₂O plus 0.1% v/v concentrated HCl) to give compound 126G (300 mg, 13.9%).

To a stirred suspension of compound 126G (300 mg, 0.28 mmol) in EtOAc (3 mL) was added 4 M HCl/EtOAc (30 mL) at 0° C. The reaction mixture was stirred at room temperature for 1 h. When the starting material was consumed, the reaction mixture was concentrated. The residue was purified by prep-HPLC (CH₃CN/H₂O plus 0.1% v/v concentrated HC) to give compound 126 (70 mg, 29.3%) as white solid. ¹H-NMR (400 MHz, MeOD-d₄) δ 7.28-7.35 (m, 2H), 7.22-7.27 (m, 2H), 7.18-7.21 (m, 1H), 4.52-4.60 (m, 1H), 4.32-4.42 (m, 1H), 4.21-4.31 (m, 3H), 4.10-4.20 (m, 1H), 2.88-3.10 (m, 6H), 2.75-2.85 (m, 1H), 2.66-2.74 (m, 1H), 2.30-2.41 (m, 1H), 2.10-2.21 (m, 1H), 1.80-1.99 (m, 7H), 1.65-1.79 (m, 7H), 1.48-1.60 (m, 5H), 1.40-1.47 (m, 4H), 1.38 (s, 3H), 1.30 (s, 3H), 1.24-1.28 (d, J=6.4 Hz, 3H), 1.14-1.17 (d, J=7.2 Hz, 3H), 0.89 (s, 3H). LCMS (ESI) for (C₄₃H₇₁BN₈O₈): m/z 839.5 (M+H).

### Example 27

### Preparation of Compound 127

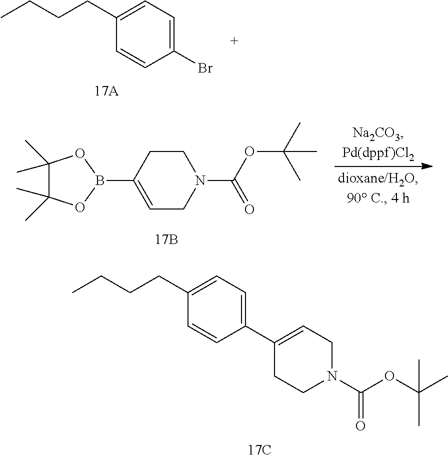

To a mixture of 1-bromo-4-butylbenzene (5 g, 23.46 mmol), compound 17B (8.71 g, 28.15 mmol) and Na₂CO₃ (4.97 g, 46.92 mmol) in dioxane (180 mL) and H₂O (45 mL) was added Pd(dppf)Cl₂ (5 g, 2.35 mmol) under N₂. The mixture was heated at 90° C. for 4 h. TLC (petroleum ether/EtOAc=5/1, Rf=0.6) showed complete consumption of the bromide. The mixture was cooled and diluted with H₂O (100 ml), and extracted with EtOAc (30 ml×3). The organic layer was dried over sodium sulfate and concentrated. The residue was purified by silica gel column chromatography (petroleum ether/EtOAc=30/1 to 10/1) to give compound 17C (5 g, 67.6%) as light yellow oil.

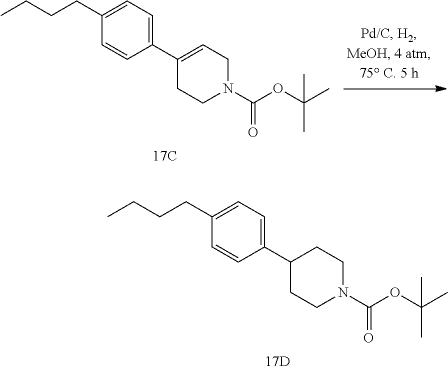

A mixture of compound 17C (5 g, 0.016 mol) and Pd/C (0.5 g, 10%) in CH₃OH (45 mL) was heated at 75° C. for 5 h under 55 psi H₂. TLC (petroleum ether/EtOAc=5/1, Rf=0.5) showed complete consumption of starting material. The mixture was concentrated to give compound 17D (5 g, 100%) as light yellow oil.

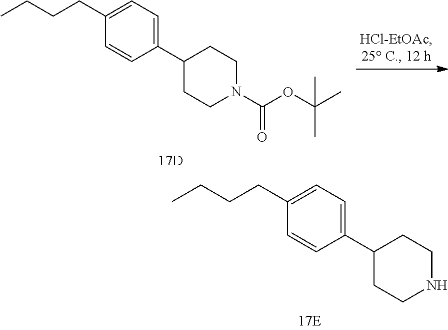

A mixture of compound 17D (5 g, 0.016 mol) in 4 M HCl in EtOAc (25 mL) was stirred at 25° C. for 12 h. TLC (petroleum ether/EtOAc=5/1, Rf=0.6) showed complete consumption of starting material. The mixture was concentrated to give compound 17E (3 g, 87%) as green solid.

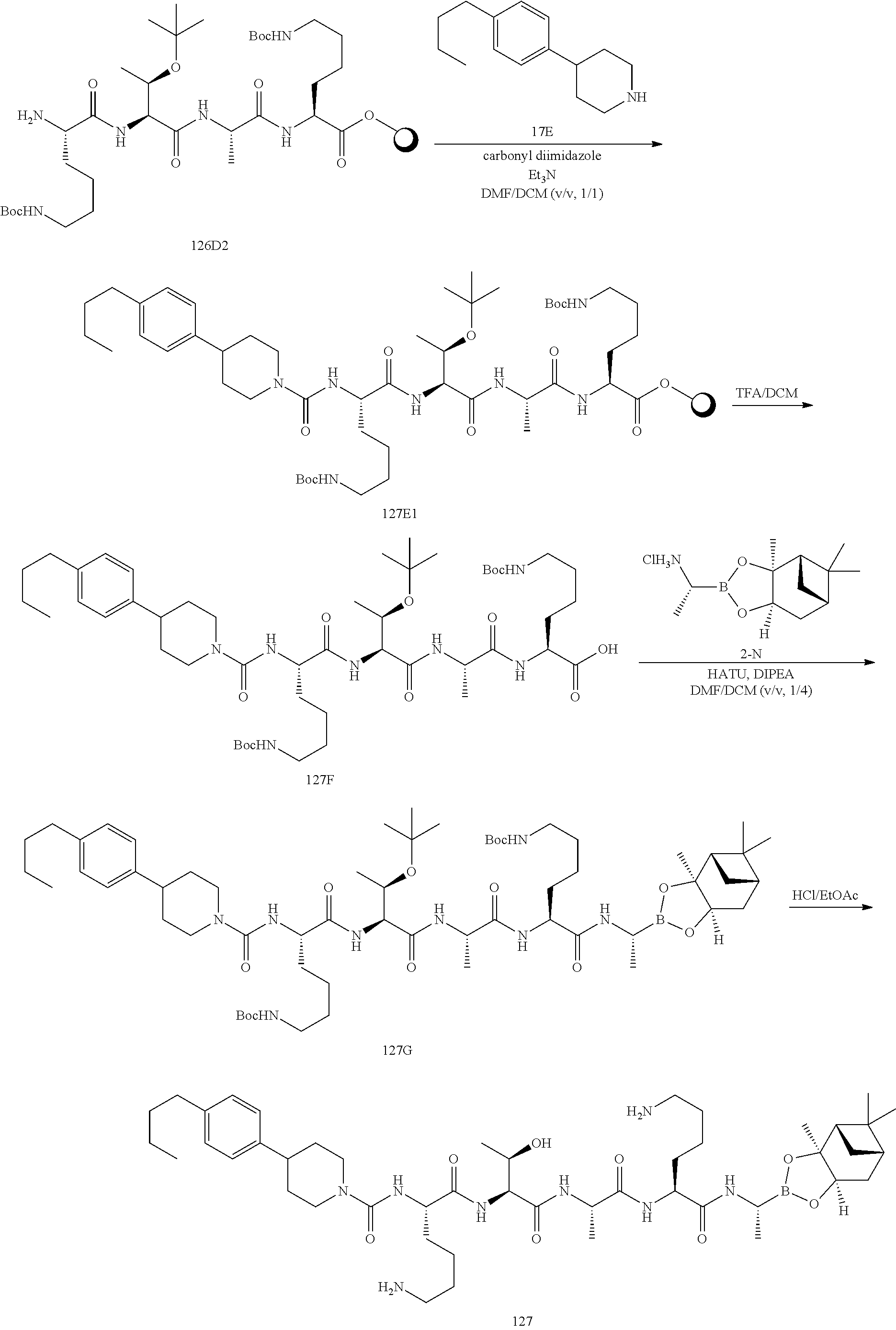

A mixture of CDI (1.49 g, 9.2 mmol) and compound 126D2 (5.6 g, 4.2 mmol) in DMF (50 mL) was stirred at 0° C. for 1 h. To the mixture was then was added Et₃N (0.93 g, 9.2 mmol) and compound 17E (1 g, 4.6 mmol) at 0° C. The reaction was stirred at room temperature for 16 h. The reaction mixture was filtered and the solid was washed with DMF/DCM (30 mL×2) and CH₃OH (20 mL) to give compound 127E1 (5 g, 76%).

A mixture of compound 127E1 (5 g, 3.2 mmol) in HOAc (5 mL)/TFE (5 mL)/DCM (40 mL) was stirred at room temperature for 5 h. The reaction mixture was filtered and the filtrate was concentrated. The residue was washed with DCM (5 mL)/petroleum ether (50 mL) to give compound 127F (1.0 g, 33%).

To a mixture of compound 127F (1.0 g, 1.06 mmol) in DMF (4 mL)/DCM (16 mL) was added HATU (0.81 g, 2.12 mmol), and (R)-BoroAla-(+)-Pinanediol HCl (0.55 g, 2.12 mmol) in an ice bath. To this mixture was added DIPEA (0.41 g, 3.18 mmol) in DMF (2.5 mL)/DCM (2.5 mL) dropwise, and the reaction was stirred for 1.5 h at 0° C. When the starting material was consumed, the reaction mixture was concentrated and the residue was poured into H₂O, extracted with EtOAc (20 mL×3), dried with Na₂SO₄ and concentrated. The crude product was purified by prep-HPLC (CH₃CN/H₂O plus HCl as an additive) to give compound 127G (50 mg, 4.5%).

To a stirred suspension of compound 127G (50 mg, 0.045 mmol) in EtOAc (3 mL) was added 4M HCl in EtOAc (30 mL) at 0° C. The reaction mixture was stirred at room temperature for 1 h. When the starting material was consumed, volatiles were removed under reduced pressure. The residue was purified by prep-HPLC (CH₃CN/H₂O plus 0.1% v/v concentrated HCl) to give 3 (20 mg, 48.9%) as white solid. ¹H-NMR (400 MHz, MeOD-d₄) δ 7.10-7.16 (m, 4H), 4.50-4.60 (m, 1H), 4.35-4.40 (m, 1H), 4.14-4.39 (m, 5H), 3.30-3.40 (m, 6H), 2.88-3.00 (m, 2H), 2.65-2.75 (m, 2H), 2.50-2.60 (m, 1H), 2.30-2.40 (m, 1H), 2.10-2.20 (m, 1H), 1.80-2.00 (m, 7H), 1.65-1.79 (m, 6H), 1.49-1.60 (m, 7H), 1.40-1.48 (m, 4H), 1.38 (s, 3H), 1.32-1.34 (m, 3H), 1.30 (s, 3H), 1.20-1.27 (d, J=8.0 Hz, 3H), 1.10-1.19 (d, J=7.2 Hz, 3H), 0.90-0.96 (t, J=7.6 Hz, 3H), 0.89 (s, 3H). LCMS (5-95 AB, ESI): RT=0.828, (M+H)⁺=895.2.

### Example 28

### Preparation of Compound 128

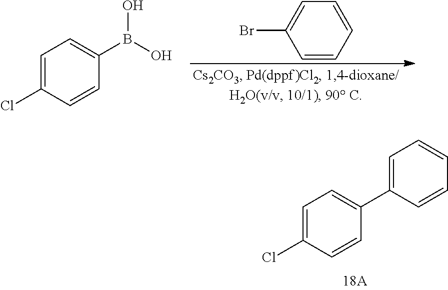 [SMILES: OB(O)c1ccc(Cl)cc1 ; Brc1ccccc1 ; Clc1ccc(-c2ccccc2)cc1]

A mixture of (4-chlorophenyl)boronic acid (13 g, 84 mol) and bromobenzene (11 g, 70 mol), Cs₂CO₃ (45 g, 140 mol), Pd(dppf)Cl₂ (5.1 g, 7 mol) in 1,4-dioxane/H₂O (165 mL, v/v, 10/1) was stirred at 90° C. overnight under N₂ atmosphere. The mixture was added water (150 mL), and the solution was extracted by EtOAc (200 mL×3). The combined organic layer were washed with brine (30 mL), dried over anhydrous Na₂SO₄ and concentrated. The residue was purified by silica gel column chromatography (petroleum ether:EtOAc 1:10) to give compound 18A (13 g, yield: 98.5%) as a yellow solid.

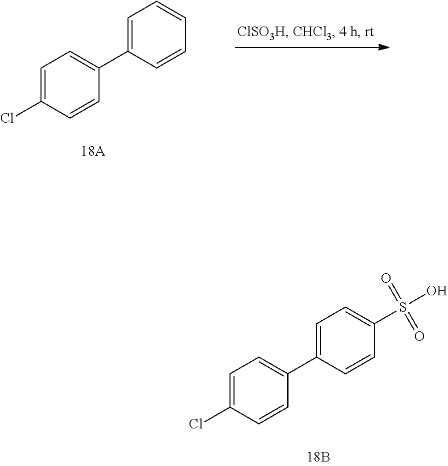 [SMILES: Clc1ccc(-c2ccccc2)cc1 ; O=S(=O)(O)c1ccc(-c2ccc(Cl)cc2)cc1]

To a solution of compound 18A (10.5 g, 55.5 mol) in chloroform (250 mL) was added ClSO₃H (9.72 g, 83.3 moL) drop-wise at 30° C. During the addition of ClSO₃H, a green white solid precipitated. The reaction mixture was stirred at 30° C. for 4 h, at which time the precipitate was collected by filtration. The product was oven-dried at 40° C. to a constant weight to give crude compound 18B (8.95 g, yield: 60.0%) as a green-white solid.

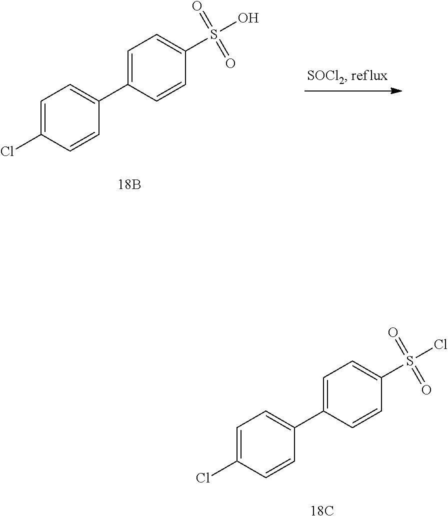 [SMILES: O=S(=O)(O)c1ccc(-c2ccc(Cl)cc2)cc1 ; O=S(=O)(Cl)c1ccc(-c2ccc(Cl)cc2)cc1]

Crude compound 18B (10.5 g, 39.07 mmol) was diluted with thionyl chloride (100 ml) and treated with a catalytic amount of DMF (0.5 ml). The reaction mixture was stirred and refluxed for 4 h. The mixture was cooled to room temperature, and concentrated in vacuo. To remove residual thionyl chloride, toluene was added to the residue and concentrated in vacuo. The resulting oily residue was recrystallized from hexane/ethyl acetate to give compound 18C (8.0 g, yield: 71.4%) as green-white solid.

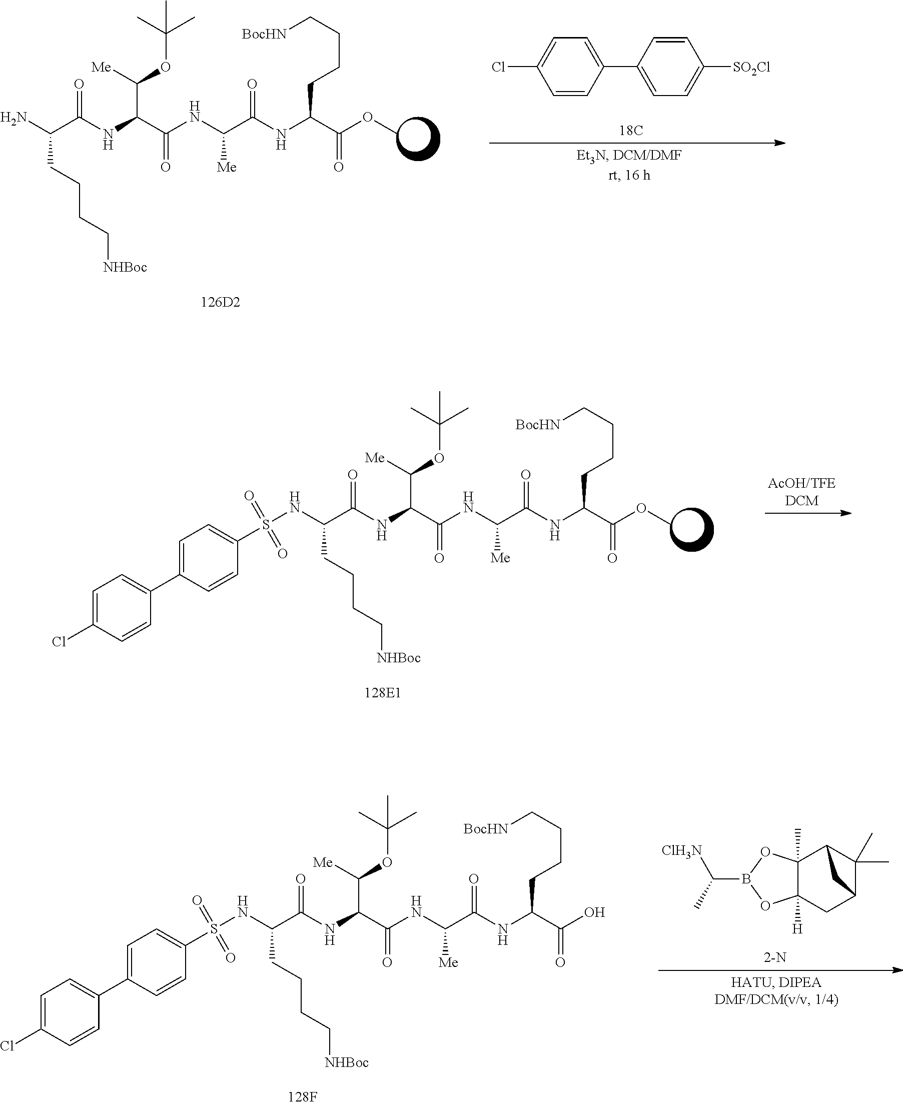

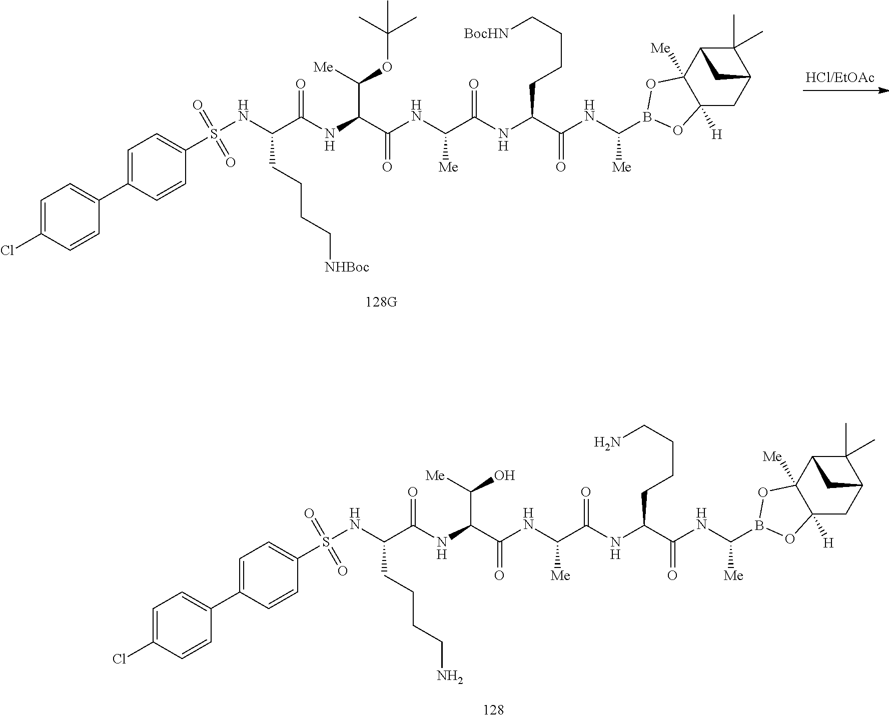 [SMILES: C[C@H](NC(=O)[C@H](CCCCNC(=O)OC(C)(C)C)NC(=O)[C@H](C)NC(=O)[C@@H](NC(=O)[C@H](CCCCNC(=O)OC(C)(C)C)NS(=O)(=O)c1ccc(-c2ccc(Cl)cc2)cc1)[C@@H](C)OC(C)(C)C)B1O[C@@H]2C[C@@H]3C[C@@H](C3(C)C)[C@]2(C)O1 ; C[C@H](NC(=O)[C@H](CCCCN)NC(=O)[C@H](C)NC(=O)[C@@H](NC(=O)[C@H](CCCCN)NS(=O)(=O)c1ccc(-c2ccc(Cl)cc2)cc1)[C@@H](C)O)B1O[C@@H]2C[C@@H]3C[C@@H](C3(C)C)[C@]2(C)O1]

To a mixture of compound 126D2 (4.64 g, 4.64 mmol) and triethylamine (1.17 g, 11.60 mmol) in DMF (20 ml)/DCM (20 ml) was added compound 18C (2.00 g, 6.96 mmol) at 0° C. The mixture was stirred at 30° C. for 15 h. The mixture was filtered and the solid was washed with DCM (20 ml)/MeOH (20 mL) to give compound 128E1 (3.8 g, 65.6%).

A mixture of compound 128E1 (3.8 g, 3.8 mmol) in DCM (32 ml)/TFE (4 ml)/AcOH (4 ml) was stirred at 30° C. for 3 h. The mixture was filtered and the solid was washed with DCM (30 mL)/MeOH (30 mL). The combined filtrates were concentrated, and the residue was washed with DCM (5 mL)/petroleum ether (50 mL) and concentrated to give compound 128F (1.5 g, 51.9%) as yellow oil.

To a mixture of compound 128F (1.5 g, 1.57 mmol) in DMF (10 mL)/DCM (30 mL) was added HATU (1.2 g, 3.14 mmol) and (R)-BoroAla-(+)-Pinanediol HCl (0.82 g, 3.14 mmol) in an ice bath. DIPEA (0.61 g, 4.72 mmol) in DMF (2.5 mL)/DCM (2.5 mL) was added after 10 min, and the reaction was stirred for 1.5 h at 0° C. When the starting material was consumed, the reaction mixture was concentrated and the residue was poured into H₂O, extracted with EtOAc (60 mL×3), dried with Na₂SO₄ and concentrated. The crude product was purified by prep-HPLC (CH₃CN/H₂O plus 0.1% v/v concentrated HCl) to give compound 128G (600 mg, 42.8%).

To a stirred suspension of compound 128G (300 mg, 0.25 mmol) in EtOAc (5 mL) was added 4M HCl in EtOAc (50 mL) at 0° C. The reaction mixture was stirred at room temperature for 1.5 h. When the starting material was consumed, the reaction mixture was concentrated. The residue was purified by prep-HPLC (CH₃CN/H₂O plus 0.1% v/v concentrated HCl) to give compound 128 (90 mg, 40%) as a white solid. ¹H-NMR (400 MHz, MeOD-d₄) δ 7.99 (d, J=8.0 Hz, 2H), 7.85 (d, J=8.0 Hz, 2H), 7.73 (d, J=7.6 Hz, 2H), 7.53 (d, J=7.2 Hz, 2H), 4.51-4.53 (m, 1H), 4.31-4.33 (m, 1H), 4.23-4.24 (m, 1H), 4.17-4.19 (m, 2H), 3.85-3.89 (m, 1H), 2.95-2.96 (m, 2H), 2.86-2.88 (m, 2H), 2.70-2.71 (m, 1H), 2.3-2.41 (m, 1H), 2.11-2.23 (m, 1H), 1.96-1.97 (m, 1H), 1.82-1.90 (m, 2H), 1.72-1.79 (m, 3H), 1.52-1.73 (m, 7H), 1.41-1.48 (m, 2H), 1.37-1.38 (m, 7H), 1.30 (s, 3H), 1.14-1.16 (m, 3H), 1.04-1.06 (d, J=6.4 Hz, 3H), 0.893 (s, 3H). LCMS (5-95 AB, ESI): RT=0.784, (M+H)⁺=902.0.

### Example 29

### Preparation of Compound 129

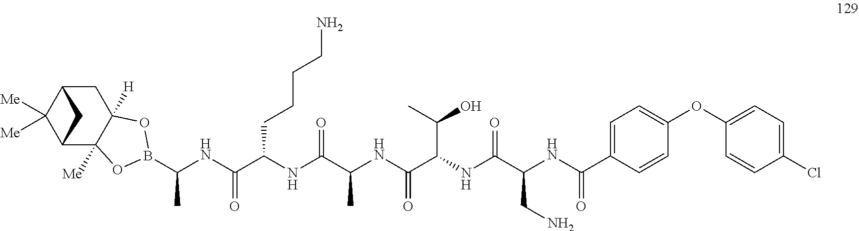 [SMILES: C[C@H](NC(=O)[C@H](CCCCN)NC(=O)[C@H](C)NC(=O)[C@@H](NC(=O)[C@H](CN)NC(=O)c1ccc(Oc2ccc(Cl)cc2)cc1)[C@@H](C)O)B1O[C@@H]2C[C@@H]3C[C@@H](C3(C)C)[C@]2(C)O1]

Compound 129 was prepared using General Methods 1 and 8. ¹H-NMR (400 MHz, MeOD-d₄) δ 0.90 (s, 3H), 1.14-1.15 (d, J=7.2 Hz, 3H), 1.19-1.21 (d, J=6.4 Hz, 3H), 1.31 (s, 3H), 1.37 (s, 3H), 1.42-1.45 (m, 7H), 1.60-1.68 (m, 2H), 1.70-1.75 (m, 2H), 1.80-1.87 (m, 2H), 1.87-1.95 (m, 2H), 2.08-2.10 (m, 1H), 2.32-2.38 (m, 1H), 2.69-2.71 (m, 1H), 2.96-3.00 (m, 3H), 3.33-3.34 (m, 2H), 3.51-3.53 (m, 1H), 4.16-4.20 (m, 1H), 4.23-4.25 (m, 1H), 4.35-4.40 (m, 1H), 4.48-4.53 (m, 1H), 4.88-4.89 (m, 1H), 4.90-4.91 (m, 1H), 7.07-7.10 (m, 4H), 7.42-7.45 (m, 2H), 7.97-8.00 (m, 2H). LCMS (5-95 AB, ESI): RT=0.751, M+H⁺=840.4.

### Example 30

### Preparation of Compound 130

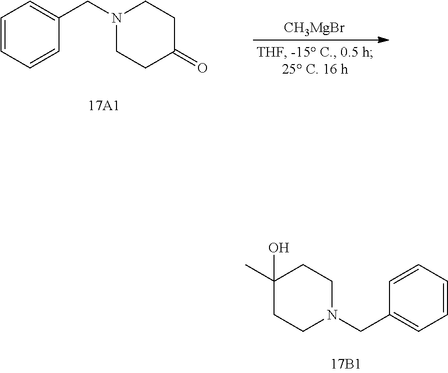

To a mixture of compound 17A1 (50 g, 264 mmol) in THF (250 mL) was added a solution of CH₃MgBr in ether (220 mL, 3M) at −15° C. under N₂ and stirred for 0.5 h, The reaction mixture was warmed to 25° C. and stirred for 16 h. TLC (petroleum ether/EtOAc=1/1, Rf=0.5) showed no starting material. The mixture was diluted with NH₄Cl (aq) (150 ml), and extracted with EtOAc (100 ml×3). The organic layer was dried over sodium sulfate and concentrated. The residue was purified by silica gel column chromatography (petroleum ether/EtOAc=3/1 to 1/1) to give compound 17B1 (18.7 g, 34.5%) as a yellow solid.

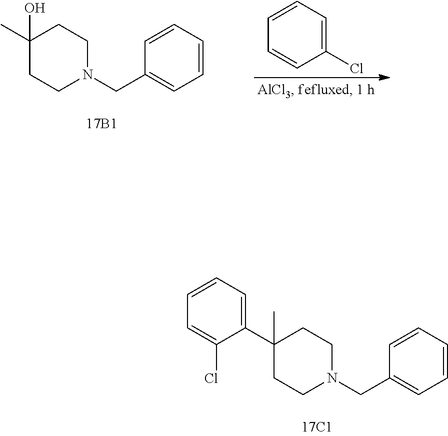 [SMILES: CC1(O)CCN(Cc2ccccc2)CC1 ; Clc1ccccc1 ; CC1(c2ccccc2Cl)CCN(Cc2ccccc2)CC1]

To a mixture of compound 17B1 (18.7 g, 91 mmol) in chlorobenzene (150 mL) was added AlCl₃ (60 g, 455 mmol). The mixture was refluxed for 1 h. TLC (petroleum ether/EtOAc=1/1, R_{f}=0.3) showed no starting material. The mixture was cooled and diluted with cold H₂O (100 ml). The mixture was treated with NaOH (1N) until basic, and then the aqueous layer was extracted with EtOAc (100 ml×3). The combined organic layer was dried over sodium sulfate and concentrated. The residue was purified by silica gel column chromatography (petroleum ether/EtOAc=3/1 to 1/1) to give compound 17C1 (9.9 g, 36.4%) as a yellow oil.

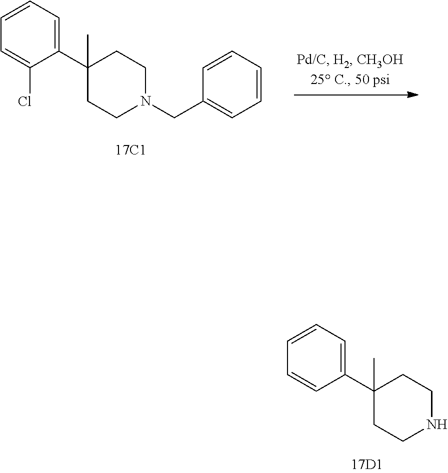 [SMILES: CC1(c2ccccc2Cl)CCN(Cc2ccccc2)CC1 ; CC1(c2ccccc2)CCNCC1]

To a mixture of compound 17C1 (9.5 g, 31.6 mmol) in CH₃OH (50 mL) was added Pd/C (1 g, 10%). The mixture was stirred at 25° C. for 72 h. TLC (petroleum ether/EtOAc=1/1, Rf=0.5) showed no starting material. The mixture was concentrated to give compound 17D1 (5.3 g, 96.3%) as yellow oil.

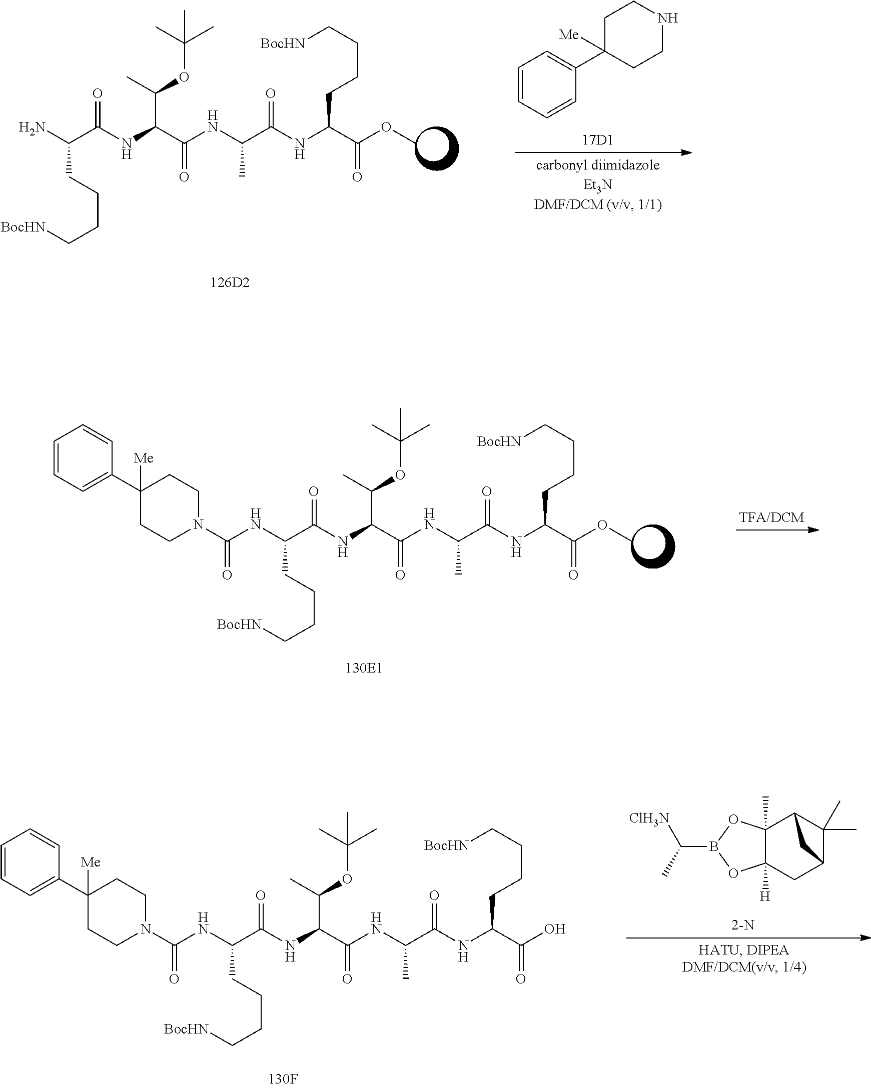

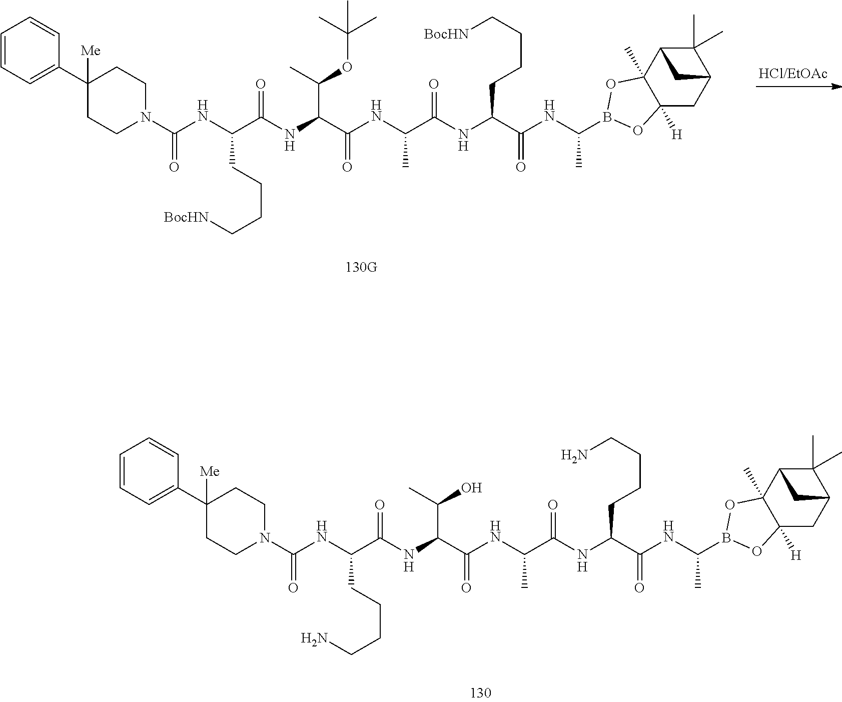 [SMILES: C[C@H](NC(=O)[C@H](CCCCNC(=O)OC(C)(C)C)NC(=O)[C@H](C)NC(=O)[C@@H](NC(=O)[C@H](CCCCNC(=O)OC(C)(C)C)NC(=O)N1CCC(C)(c2ccccc2)CC1)[C@@H](C)OC(C)(C)C)B1O[C@@H]2C[C@@H]3C[C@@H](C3(C)C)[C@]2(C)O1 ; C[C@H](NC(=O)[C@H](CCCCN)NC(=O)[C@H](C)NC(=O)[C@@H](NC(=O)[C@H](CCCCN)NC(=O)N1CCC(C)(c2ccccc2)CC1)[C@@H](C)O)B1O[C@@H]2C[C@@H]3C[C@@H](C3(C)C)[C@]2(C)O1]

A mixture of CDI (1.49 g, 9.2 mmol) and compound 126D2 (6 g, 4.0 mmol) in DMF (50 mL) was stirred at 0° C. for 1 h. To the reaction mixture was then added Et₃N (0.93 g, 9.2 mmol) and compound 17D1 (2 g, 11.4 mmol) at 0° C. The reaction mixture was stirred at room temperature for 16 h. The reaction mixture was filtered and the solid was washed with DMF/DCM (50 mL×2), then CH₃OH (20 mL) to give compound 130E1 (6 g, 88.2%).

A mixture of compound 130E1 (6 g, 4.0 mmol) in HOAc (5 mL)/TFE (5 mL)/DCM (40 mL) was stirred at room temperature for 5 h. The reaction mixture was filtered and the filtrate was concentrated, the residue was washed by DCM (5 mL)/petroleum ether (50 mL) to give compound 130F (0.5 g, 20.8%).

To a mixture of compound 130F (0.5 g, 0.55 mmol)) in DMF (4 mL)/DCM (16 mL) was added HATU (0.42 g, 1.11 mmol), (R)-BoroAla-(+)-Pinanediol HCl (0.29 g, 1.11 mmol) in an ice bath, DIPEA (0.21 g, 1.65 mmol) in DMF (2.5 mL)/DCM (2.5 mL) was added after 10 min and the reaction was stirred for 1.5 h at 0° C. When the starting material was consumed, the reaction mixture was concentrated and the residue was poured into H₂O, extracted by EtOAc (20 mL×3), dried with Na₂SO₄ and concentrated. The crude product was purified by pre-HPLC under HCl condition to give compound 130G (300 mg, 49.2%).

To a stirred suspension of compound 130G (300 mg, 0.27 mmol) in EtOAc (3 mL) was added 4M HCl in EtOAc (30 mL) at 0° C., the reaction mixture was stirred at room temperature for 1 h. When the starting material was consumed, the reaction mixture was concentrated. The residue was purified by prep-HPLC (CH₃CN/H₂O plus 0.1% v/v concentrated HCl) to give compound 130 (60 mg, 26%) as white solid. ¹H-NMR (400 MHz, MeOD-d₄) δ 7.36-7.41 (m, 2H), 7.30-7.35 (m, 2H), 7.15-7.21 (m, 1H), 4.51-4.60 (m, 1H), 4.31-4.40 (m, 1H), 4.24-4.30 (m, 1H), 4.20-4.23 (m, 1H), 4.10-4.19 (m, 2H), 3.60-3.70 (m, 2H), 3.30-3.40 (m, 1H), 2.88-3.00 (m, 4H), 2.65-2.75 (m, 1H), 2.30-2.40 (m, 1H), 2.10-2.21 (m, 3H), 1.91-2.00 (m, 1H), 1.81-1.90 (m, 3H), 1.71-1.80 (m, 4H), 1.60-1.70 (m, 6H), 1.46-1.52 (m, 4H), 1.40-1.45 (m, 4H), 1.38 (s, 3H), 1.31 (s, 3H), 1.28 (s, 3H), 1.20-1.24 (d, J=6.0 Hz, 3H), 1.14-1.16 (d, J=7.2 Hz, 3H), 0.89 (s, 3H), LCMS (5-95 AB, ESI): RT=0.768, (M+H)⁺=853.5.

### Example 31

### Preparation of Compound 131

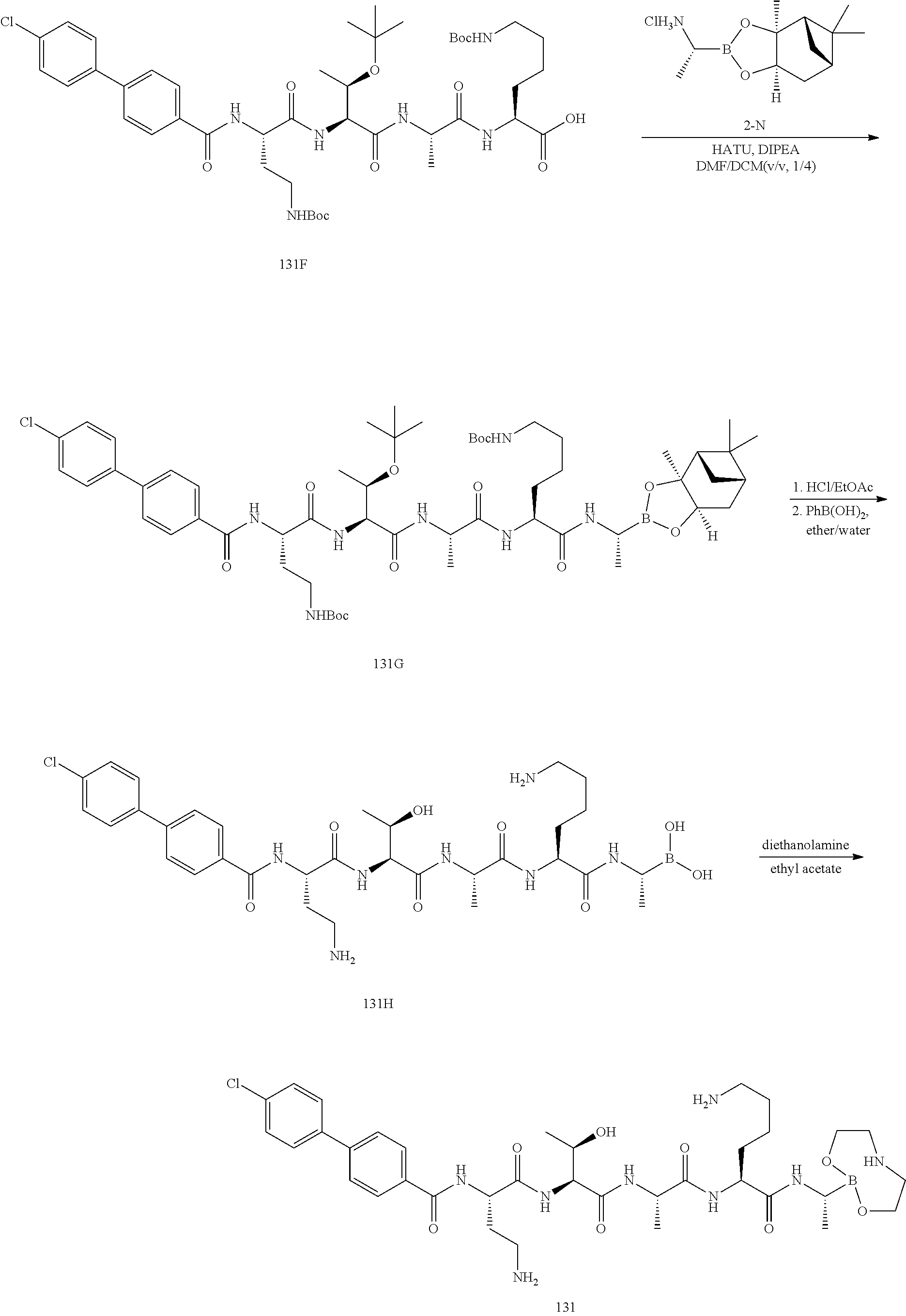

Compound 131H was prepared from compound 131F using General Methods 1 and 8 followed by pinanediol removal by treatment with PhB(OH)₂ according to the procedure used to prepare compound 119 to afford compound 131H. Diethanolamine (5 equiv., 0.461 mmol, 48 mg) was added to a solution of 131H (65 mg, 0.09 mmol) in ethyl acetate (3 mL) and the mixture was stirred at room temperature for 2 hours. The reaction was then concentrated and the residue was purified by preparative HPLC to give 131 as a white solid. LCMS (5-95 AB, ESI): RT=2.92, M+H⁺=704.3 [hydrolyzed to the B(OH)₂].

### Example 32

### Preparation of Compound 132

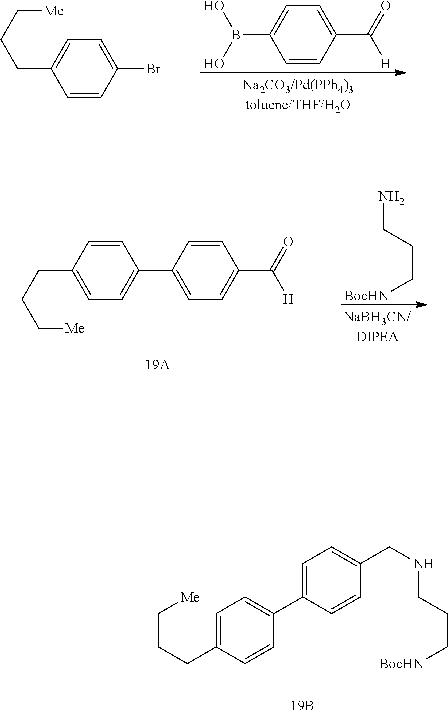 [SMILES: CCCCc1ccc(Br)cc1 ; O=Cc1ccc(B(O)O)cc1 ; CCCCc1ccc(-c2ccc(C=O)cc2)cc1 ; CC(C)(C)OC(=O)NCCCN ; CCCCc1ccc(-c2ccc(CNCCCNC(=O)OC(C)(C)C)cc2)cc1]

A solution of 1-bromo-4-butylbenzene (50.0 g, 0.333 mol), 4-formylphenylboronic acid (47.2 g, 0.222 mol), Na₂CO₃ (70.6 g, 0.666 mol) in toluene/THF/H₂O (200 mL/200 mL/200 mL) was degassed with N₂ three times, and then Pd(PPh₃)₄ (12.8 g, 11.2 mmol) was added. The resulting mixture was degassed with N₂ three times and then heated to reflux for 5 h. After TLC showed the reaction was complete, toluene and THF was removed under vacuum. The residue was extracted with EA (30 mL×3). The combined organic layers were washed with brine, and dried with Na₂SO₄. The solvent was removed to give the crude product. The crude product was purified by column chromatography on silica gel eluted with PE. The solvent was removed to give compound 19A (20.0 g, yield: 37.8%), as a yellow oil.

To a solution of compound 19A (0.900 g, 2.31 mmol) in dry DCM (30 mL) was added tert-butyl (3-aminopropyl)carbamate (0.575 g, 2.42 mmol), DIPEA (0.672 g, 5.21 mmol) and Na₂SO₄ (6 g) at 15° C. The mixture was stirred for 2 hrs at 15° C. The mixture was filtered and the filtrate was evaporated, dissolved in dry MeOH (30 mL), and cooled to 0° C. To the solution was added NaBH₃CN (96.6 mg, 2.54 mmol) portion-wise, and then the mixture was stirred for 1.5 hrs at 15° C. After TLC showed the reaction was complete, the solvent was evaporated and the crude product was purified by column chromatography on silica gel eluted with DCM: MeOH (10:1). The solvent was removed to give compound 19B (1.70 g, yield: 48%). MS (ESI) m/z 397.1 (M+H)⁺.

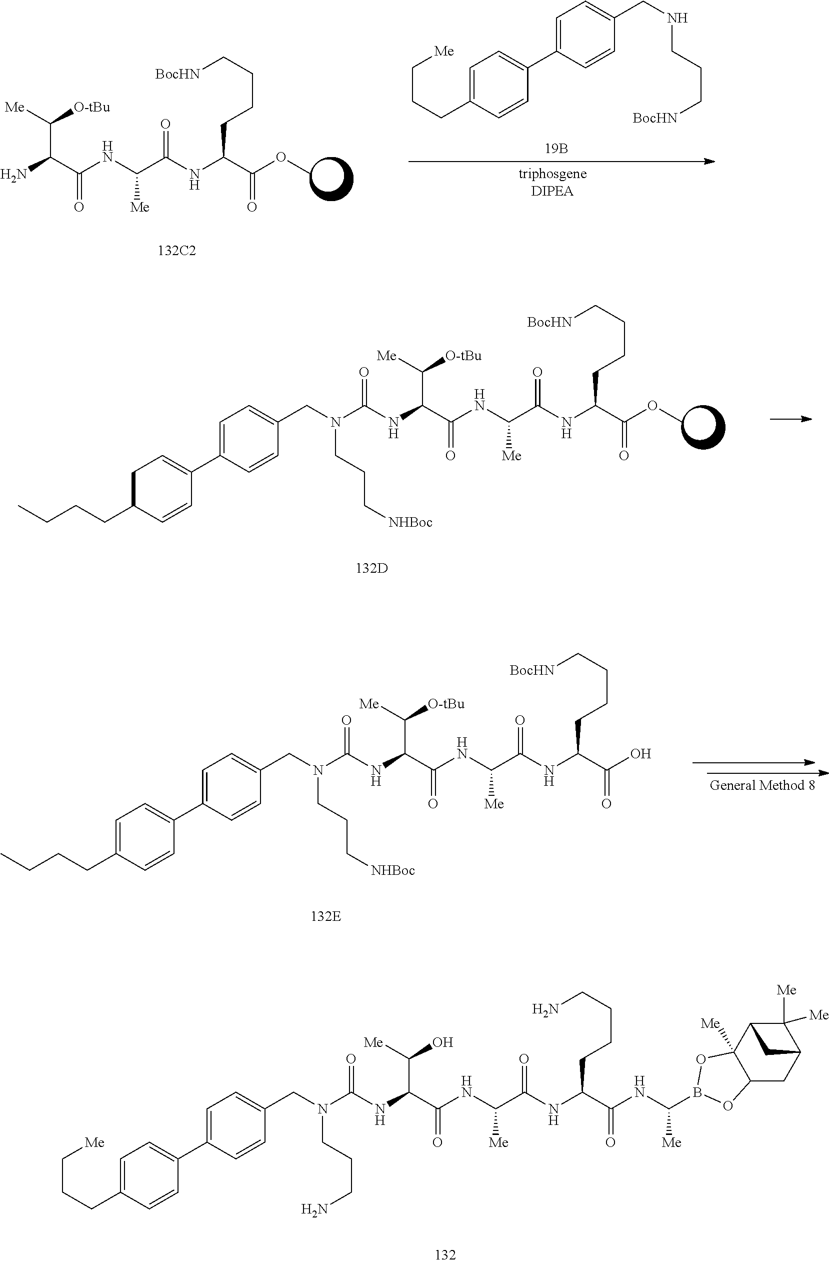

Compound 132C2 was prepared according to General Method 1. To a solution of triphosgene (20.6 mg, 0.07 mmol) in dry DCM (3 mL) was slowly added a solution of compound 132C2 (0.23 mmol) and DIPEA (297 mg, 2.30 mmol) in THF (3 mL) at 0° C. The reaction mixture was stirred at 15° C. for 25 mins. A solution of compound 19B (91.36 mg, 2.30 mmol) was added at 0° C. in THF (2 mL) and the reaction mixture was warmed to 15° C. and shaken at 15° C. for 4 hrs. After LCMS showed the reaction was completed, the mixture was filtered. The filter cake was washed sequentially with THF (20 mL×3) and DCM (20 mL×3), and then dried under vacuum to afford compound 132D. TFA/DCM (1%, 5 mL) was added and the mixture was shaken at 15° C. for 5 mins. The mixture was filtered and the filtrate was treated with saturated NaHCO₃ solution until a pH of 7-8 was obtained. The aqueous layer was adjusted with citric acid until pH=3-4. The mixture was extracted with DCM (20 mL×3). The combined organic layers were washed with brine, dried over Na₂SO₄ and concentrated to give compound 132E (80.0 mg, yield: 38.8%). MS (ESI) m/z 897.4 (M+H)⁺.

Compound 132E was subjected to the conditions described in General Method 8 to afford compound 132. MS (ESI) m/z 846.5 (M+H)⁺.

### Example 33

### Preparation of Compound 133

To a solution of compound 126D2 (2.63 g, 2.63 mmol) and triethylamine (0.66 g, 6.58 mmol) in DMF (10 ml)/DCM (10 ml) was added compound 18C1 (1.0 g, 3.94 mmol) at 0° C. The mixture was stirred at 30° C. for 16 h. The mixture was filtered and the solid was washed with DCM (20 ml)/MeOH (20 mL) to give compound 133E1 (2.1 g, 64%). Compound 133 was prepared according to General Methods 1 and 8 from Compound 133E1. ¹H NMR (400 MHz, MeOD-d₄) δ 7.97-7.99 (d, J=8.0 Hz, 2H), 7.83-7.86 (d, J=8.0 Hz, 2H), 7.70-7.73 (d, J=7.6 Hz, 2H), 7.48-7.57 (m, 2H), 7.43-7.46 (d, J=7.2 Hz, 1H), 4.50-4.56 (m, 1H), 4.31-4.39 (m, 1H), 4.14-4.27 (m, 3H), 3.85-3.95 (m, 1H), 2.96-3.00 (m, 2H), 2.82-2.91 (m, 2H), 2.65-2.75 (m, 1H), 2.29-2.42 (m, 1H), 2.10-2.21 (m, 1H), 1.93-1.99 (m, 1H), 1.88-1.92 (m, 2H), 1.75-1.84 (m, 3H), 1.69-1.73 (m, 3H), 1.62-1.64 (m, 2H), 1.48-1.52 (m, 2H), 1.34-1.46 (m, 9H), 1.30 (s, 3H), 1.12-1.17 (m, 3H), 1.05-1.08 (d, J=6.4 Hz, 3H), 0.89 (s, 3H). LCMS (5-95 AB, ESI): RT=0.695, (M/2-41)⁺=435.0.

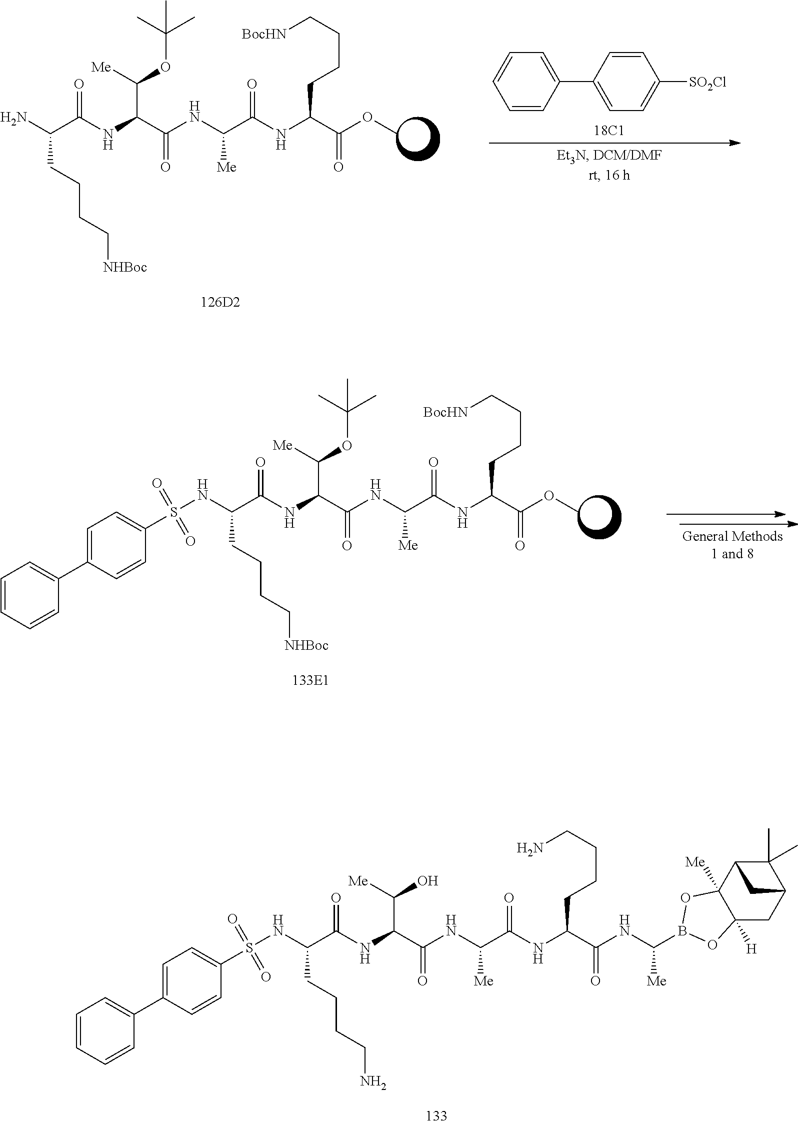 [SMILES: COC(=O)[C@H](CCCCNC(=O)OC(C)(C)C)NC(=O)[C@H](C)NC(=O)[C@@H](NC(=O)[C@@H](N)CCCCNC(=O)OC(C)(C)C)[C@@H](C)OC(C)(C)C ; O=S(=O)(Cl)c1ccc(-c2ccccc2)cc1 ; COC(=O)[C@H](CCCCNC(=O)OC(C)(C)C)NC(=O)[C@H](C)NC(=O)[C@@H](NC(=O)[C@H](CCCCNC(=O)OC(C)(C)C)NS(=O)(=O)c1ccc(-c2ccccc2)cc1)[C@@H](C)OC(C)(C)C ; C[C@H](NC(=O)[C@H](CCCCN)NC(=O)[C@H](C)NC(=O)[C@@H](NC(=O)[C@H](CCCCN)NS(=O)(=O)c1ccc(-c2ccccc2)cc1)[C@@H](C)O)B1O[C@@H]2C[C@@H]3C[C@@H](C3(C)C)[C@]2(C)O1]

### Example 34

### Preparation of Compound 201 P1

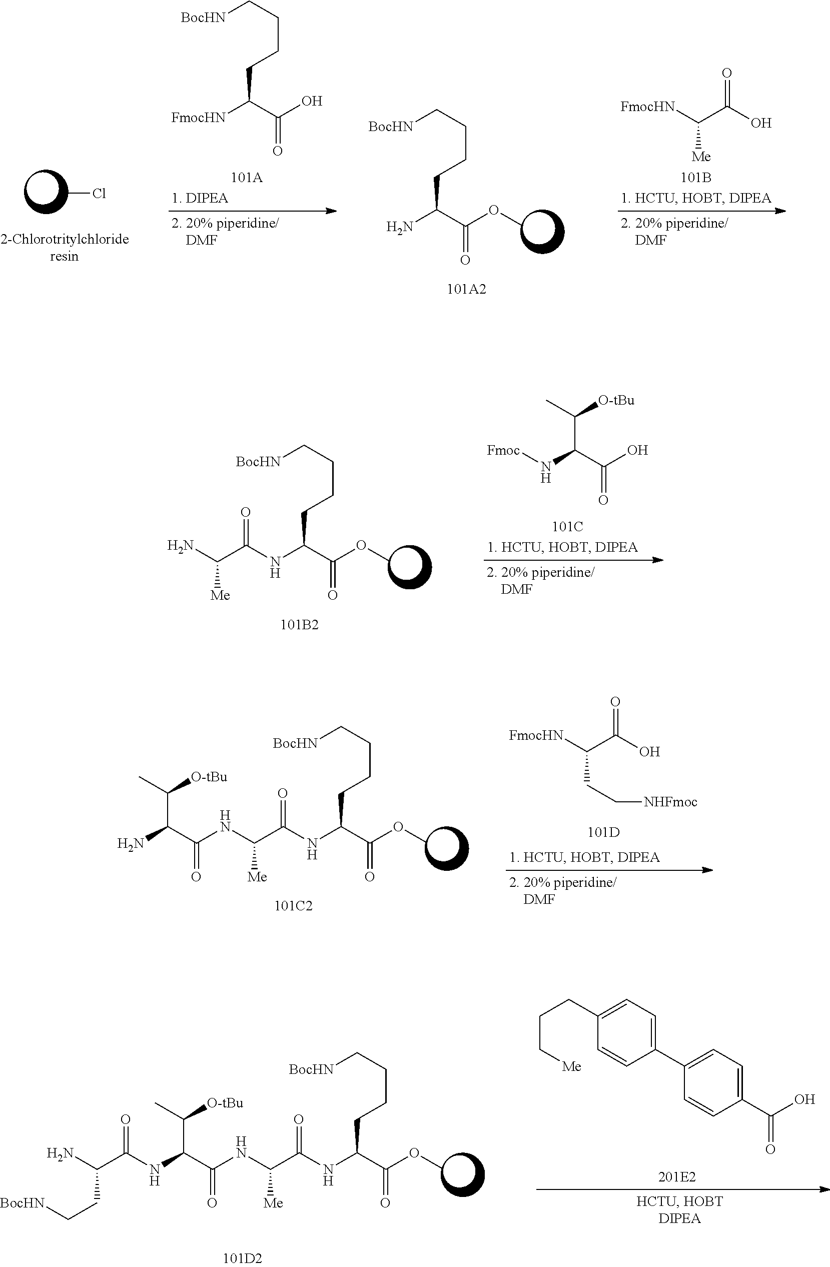

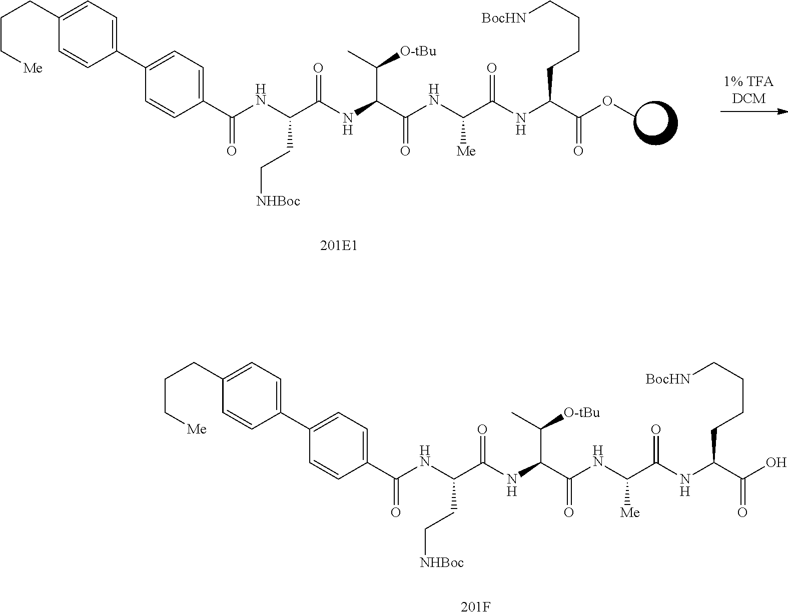

General Method 9:

The preparation of Compound 201F utilizes sequential solid phase peptide coupling and subsequent Fmoc-deprotection and is referred to as General Method 9.

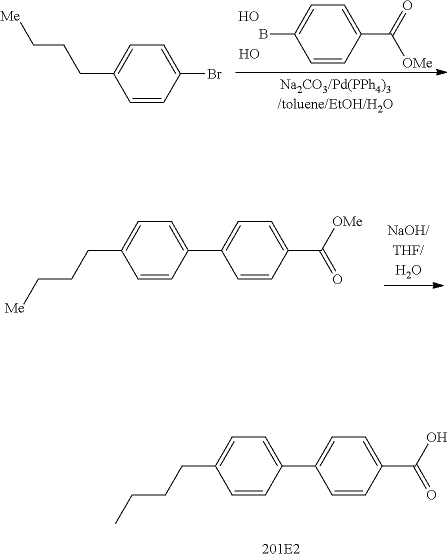

A solution of 1-bromo-4-butylbenzene (100 g, 0.472 mol), 4-(methoxycarbonyl)phenylboronic acid (82.0 g, 0.456 mol), 2 M Na₂CO₃ (150 g, 1.42 mol) in toluene/EtOH (900 mL/300 mL) was degassed with N₂ three times, then Pd(PPh₃)₄ (27.2 g, 23.6 mmol) was added. The resulting mixture was degassed with N₂ three times and then heated to reflux for 5 hrs. After TLC showed the reaction was complete, toluene and EtOH was removed under vacuum. The residue was extracted with EA (30 mL×3). The combined organic layers were washed with brine, dried with Na₂SO₄. The solvent was removed to give the crude product. The crude product was purified by column chromatography on silica gel eluted with PE, PE: EA (150:1). The solvent was removed to give methyl 4-(4-butylphenyl)benzoate (105 g, yield: 86.0%), as a white solid.

A mixture of methyl 4-(4-butylphenyl)benzoate (89.0 g, 0.332 mol), NaOH (26.6 g, 0.664 mol) in THF/H₂O (500 mL/100 mL) was heated to reflux overnight. After TLC showed the reaction was complete, THF was removed. The residue was adjusted pH ˜3-4 with 2 N HCl solution. The resulting mixture was filtered and the cake was washed with water, dried to give 4-(4-butylphenyl)benzoic acid (Compound 201E2) (60.0 g, yield: 71.1%), as a white solid. (ESI) m/z 255.0 (M+H)⁺.

A mixture of 4-(4-butylphenyl)benzoic acid (2 mmol), HCTU (2 mmol), HOBT (2 mmol) and DIPEA (2 mmol) in dry DMF (20 mL) was stirred at 20° C. for 30 min. Then the above mixture was added to Compound 101D2 (prepared as in Example 1) and shaken at 20° C. for 1.5 hrs. After LCMS showed the reaction was completed, the mixture was filtered and the residue was washed with DMF (3×30 mL) and DCM (2×30 mL) to give Compound 201E1.

A mixture of Compound 201E1 (1 mmol) was treated with 1% TFA/DCM (4 mL) for 5 min and filtered. This operation was repeated three times. The filtrate was treated with saturated NaHCO₃ solution until pH ˜7-8. The aqueous layer was adjusted to pH ˜3-4 with citric acid. The mixture was extracted with DCM (8 mL) three times, and then the combined organic layers were washed with brine, dried over Na₂SO₄ and concentrated to give Compound 201F. MS (ESI) m/z 911.4 (M+H)⁺.

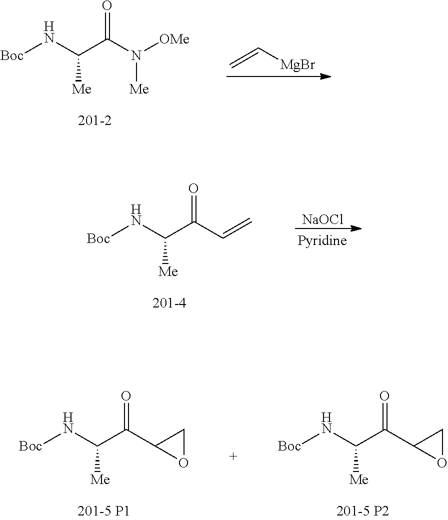

A solution of Compound 201-2 (1 g, 4.3 mmol, 1 eq) in anhydrous THF (76 mL) was cooled to −78° C. and a 1 M solution of vinyl magnesium bromide (9.1 mL, 2.1 eq) was added dropwise over 15 mins. The solution was then warmed to 0° C. on an ice bath. After stirring for 2 hrs, TLC indicated complete consumption of starting material and the reaction mixture was poured into stirring 1 N HCl (30 mL) at 0° C., the mixture was then diluted with an equivalent amount of water and extracted 3× with EtOAc. The combined organic layers were then washed with brine, dried over Na₂SO₄ and concentrated. The crude material was purified via flash chromatography (0 to 50% EtOAc in hexanes) to afford Compound 201-4 (706 mg, 82%). R_{f} 0.6 (25% EtOAc/hexanes). ¹H NMR (CDCl₃, 500 MHz) δ 6.47 (dd, (J=15 Hz, 10 Hz), 1H) 6.38 (dd, (J=18 Hz, 1.5 Hz), 1H), 5.85 (d, J=10 Hz, 1H), 5.35 (br s, 1H), 4.64-4.61 (m, 1H), 1.44 (s, 9H), 1.34 (d, J=7 Hz, 3H).

To a solution of Compound 201-4 (250 mg, 1.3 mmol, 1 eq) in pyridine (5 mL) at −10° C. was added a 10% solution of aqueous NaOCl (1.87 mL, 2 eq) dropwise over 10 mins. The reaction was then warmed to 0° C. and allowed to stir for 2 hrs at which time TLC indicated the reaction was completed. The reaction was then diluted with EtOAc at 0° C., the organic layer was washed twice with water and brine, dried over sodium sulfate and concentrated. The crude material was purified via flash chromatography (0 to 50% EtOAc in hexanes) to give two products, Compound 201-5 P1 (106 mg, R_{f} 0.5 (25% EtOAc in hexanes)) and Compound 201-5 P2 (63 mg, R_{f} 0.2 (25% EtOAc in hexanes)) (62% combined yield). Data for Compound 201-5 P1: ¹H NMR (CDCl₃, 500 MHz) δ 5.04 (m, 1H), 4.31-4.29 (m, 1H), 3.54-3.52 (m, 1H), 3.10-3.09 (m, 1H), 3.06-3.04 (m, 1H), 1.42 (s, 9H), 1.31 (d, J=7 Hz, 3H). Data for Compound 201-5 P2: ¹H NMR (CDCl₃, 500 MHz) δ 5.14 (m, 1H), 4.58-4.50 (m, 1H), 3.68 (dd, J=4.5 Hz, 2.5 Hz, 1H), 3.00 (dd, J=6.5 Hz, 4.5 Hz, 1H), 2.91 (dd, J=6.5 Hz, 2.5 Hz), 1.44 (s, 9H), 1.39 (d, J=7 Hz, 3H).

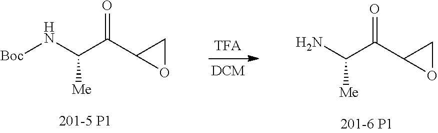 [SMILES: C[C@H](NC(=O)OC(C)(C)C)C(=O)C1CO1 ; C[C@H](N)C(=O)C1CO1]

General Method 10:

TFA hydrolysis of a Boc-protected amino acid. A boc-protected amino acid is dissolved in DCM (0.02-0.2 M) and cooled to 0° C. TFA is added to dropwise to create a 4:1 ratio of DCM:TFA. The solution is stirred for 15 minutes or until LC-MS analysis shows the reaction to be completed. The TFA and DCM are removed under reduced pressure to afford the desired amine. Compound 201-6 P1 was prepared according to General Method 10.

General Method 11:

Coupling of an amino-epoxyketone to a carboxylic acid followed by global acidic deprotection. To a solution of the epoxyketone monomer (1.5 eq) in a 3:1 mixture of DCM and DMF was added the peptide carboxylic acid (1 eq) followed by HATU (1.5 eq) then DIPEA (20 eq) at room temperature. The solution was stirred for 2 hrs then diluted with 0.5 M HCl and extracted three times with DCM. The combined organic layers were then washed with H₂O and saturated NaHCO₃, dried over sodium sulfate and concentrated. The product was then purified by silica gel column chromatography. The protected epoxyketone-peptide conjugate was then treated with a 4:1 mixture of DCM:TFA at 0° C. The solution was stirred until LCMS analysis showed complete conversion to the product (˜2 hrs) then the solvents were evaporated and the residue was azeotroped twice with DCM and dried under vacuum. The crude residue was taken up in MeOH, centrifuged to remove insoluble particulates and purified via HPLC to give the product.

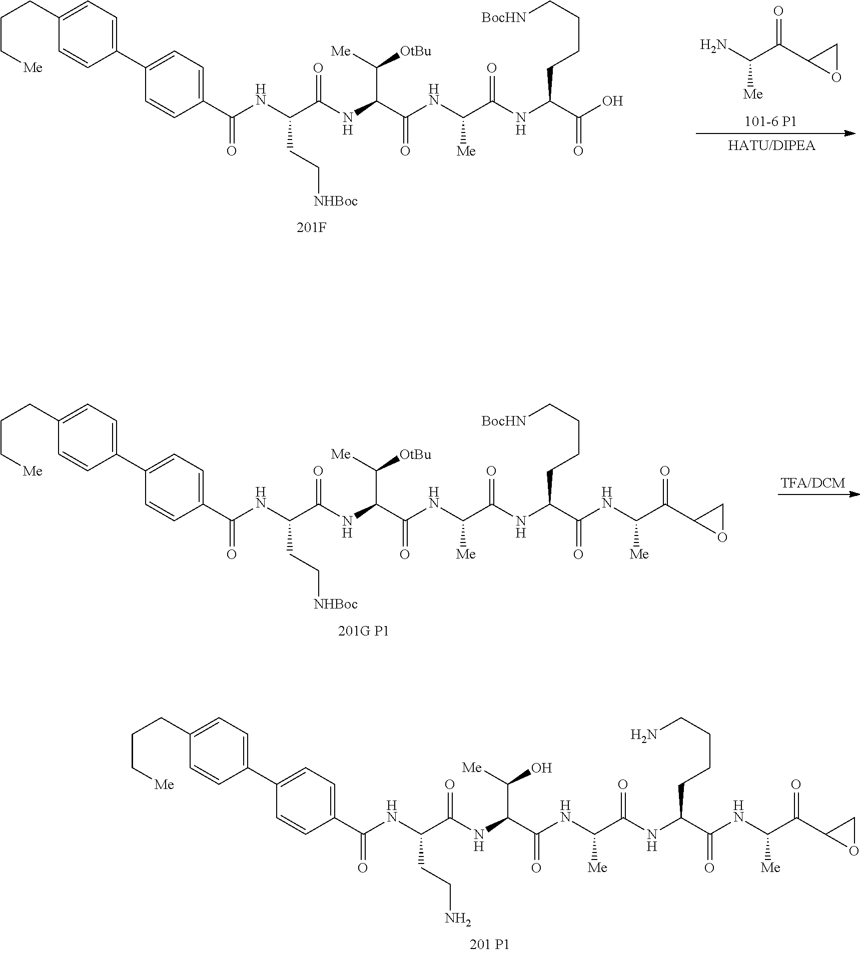 [SMILES: CCCCc1ccc(-c2ccc(C(=O)N[C@@H](CCNC(=O)OC(C)(C)C)C(=O)N[C@H](C(=O)N[C@@H](C)C(=O)N[C@@H](CCCCNC(=O)OC(C)(C)C)C(=O)O)[C@@H](C)OC(C)(C)C)cc2)cc1 ; C[C@H](N)C(=O)C1CO1 ; CCCCc1ccc(-c2ccc(C(=O)N[C@@H](CCNC(=O)OC(C)(C)C)C(=O)N[C@H](C(=O)N[C@@H](C)C(=O)N[C@@H](CCCCNC(=O)OC(C)(C)C)C(=O)N[C@@H](C)C(=O)C3CO3)[C@@H](C)OC(C)(C)C)cc2)cc1 ; CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)C3CO3)[C@@H](C)O)cc2)cc1]

Compound 201 P1 was prepared according to General Method 11 from Compound 201F and Compound 101-6 P1. Data for Compound 201 P1: MS (ESI) m/z 752.3 (M+H)⁺; t_{R} 4.10 min (10% CH₃CN/H₂O−90% CH₃CN/H₂O, 6.5 min, 1.0 mL/min Gemini-NX C18, 4.6×50 mm).

### Example 35

### Preparation of Compound 201 P2

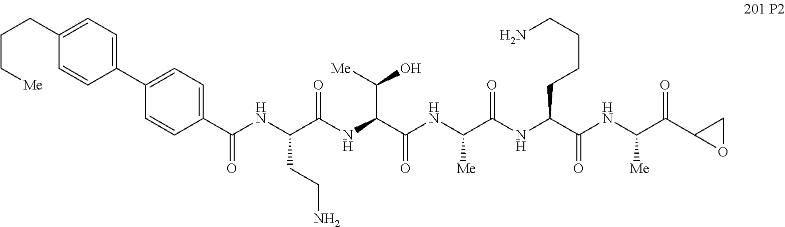 [SMILES: CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)C3CO3)[C@@H](C)O)cc2)cc1]

Compound 201 P2 was prepared according to General Methods 10 and 11 (Example 34) from 201F and 201-5 P2. MS (ESI) m/z 752.1 (M+H)⁺; t_{R} 4.15 min (10% CH₃CN/H₂O−90% CH₃CN/H₂O, 6.5 min, 1.0 mL/min Gemini-NX C18, 4.6×50 mm).

### Example 36

### Preparation of Compound 203 P1

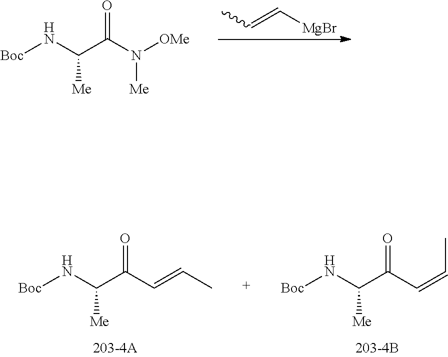

A solution of 1-propenyl magnesium bromide (0.5 M in THF, 45 mL) was added dropwise to Boc-L-Ala-N(OMe)(Me) (1.74 g, 7.5 mmol) in 15 mL THF at −5° C. The reaction was stirred at −5° C. for 1.5 h, then was poured into a cold mixture of ether/0.2 N NaHSO₄. The mixture was extracted with ether, and the combined organic layers were washed sequentially with saturated NaHCO₃ and brine, dried over Na₂SO₄, filtered and concentrated. Flash chromatography (10% EtOAc/hexanes-50% EtOAc/hexanes) afforded 0.35 g (22%) of Compound 203-4A and 0.70 g (44%) of 103-4A. Data for Compound 203-4A: ¹H NMR (CDCl₃) δ 7.02 (dq, J=15.5, 7.0, 1H), 6.22 (broad d, J=15.5, 1H), 5.37 (broad s, 1H), 4.5-4.6 (m, 1H), 1.93 (dd, J=7.0, 1.5, 3H), 1.44 (s, 9H), 1.32 (d, J=7.0, 3H). R_{f} 0.32 (4:1 hexanes:EtOAc). Data for Compound 203-4B: ¹H NMR (CDCl₃, 500 MHz) δ 6.37 (dq, J=11.5, 7.5, 3H), 6.21 (broad d, J=11.5, 1H), 5.37 (broad s, 1H), 4.3-4.4 (m, 1H), 2.15 (dd, J=7.5, 1.8, 3H), 1.44 (s, 9H), 1.32 (d, J=7.0, 3H). R_{f} 0.38 (4:1 hexanes:EtOAc).

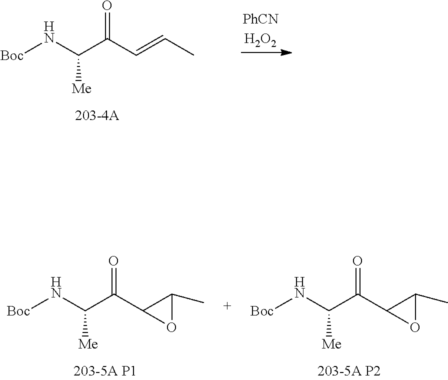 [SMILES: C/C=C/C(=O)[C@H](C)NC(=O)OC(C)(C)C ; CC1OC1C(=O)[C@H](C)NC(=O)OC(C)(C)C ; CC1OC1C(=O)[C@H](C)NC(=O)OC(C)(C)C]

A solution of 50% H₂O₂ (0.13 mL, 2.2 mmol) was added to a solution of Compound 103-4A (64 mg, 0.30 mmol) and benzonitrile (0.23 mL, 2.2 mmol) in 3 mL methanol at 0° C. DIPEA (0.39 mL, 2.2 mmol) was added, and the reaction was stirred for 3 h at 0° C., whereupon it was warmed to room temperature and stirred for 30 min. The methanol was evaporated, and the mixture partitioned between ether and 0.2 N NaHSO₄. The organic layer was washed sequentially with saturated NaHCO₃ and brine, dried over Na₂SO₄, filtered and concentrated. Flash chromatography (10% EtOAc/hexanes-40% EtOAc/hexanes) afforded 17 mg (24%) of Compound 203-5A P1 and 12 mg (17%) of Compound 203-5A P2. Data for Compound 203-5A P1: ¹H NMR (CDCl₃, 500 MHz) δ 5.0-5.1 (m, 1H), 4.2-4.3 (m, 1H), 3.29-3.36 (m, 1H), 3.28 (broad s, 1H), 1.43 (d, 3H), 1.42 (s, 9H), 1.29 (d, J=7.5, 3H). R_{f} 0.42 (3:1 hexanes:EtOAc). Data for Compound 203-5A P2: ¹H NMR (CDCl₃, 500 MHz) δ 5.1-5.2 (broad s, 1H), 4.45-4.55 (m, 1H), 3.43 (d, J=1.5, 1H), 3.1-3.2 (m, 1H), 1.42-1.47 (12H, N-Boc, CH₃), 1.38 (d, 3H). R_{f} 0.25 (3:1 hexanes:EtOAc).

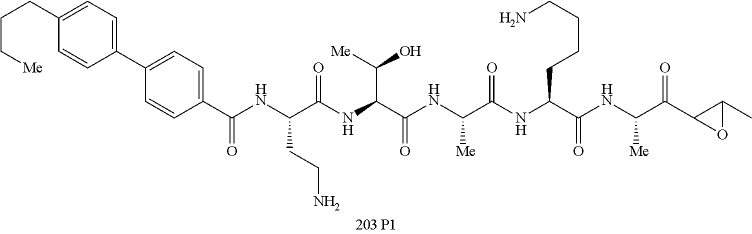 [SMILES: CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)C3OC3C)[C@@H](C)O)cc2)cc1]

Compound 203 P1 was prepared according to General Methods 10 and 11 (Example 34) from Compound 201F and Compound 203-5A P1. MS (ESI) m/z 766.3 (M+H)⁺; t_{R} 4.17 min (10% CH₃CN/H₂O−90% CH₃CN/H₂O, 6.5 min, 1.0 mL/min Gemini-NX C18, 4.6×50 mm).

### Example 37

### Preparation of Compound 203 P2

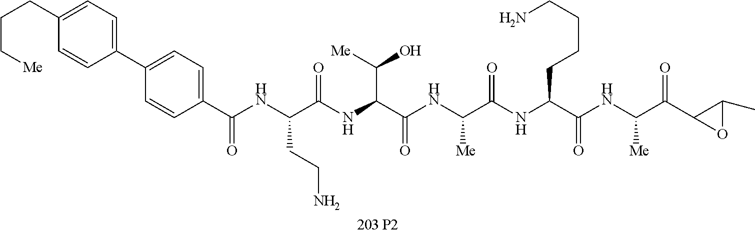 [SMILES: CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)C3OC3C)[C@@H](C)O)cc2)cc1]

Compound 203 P2 was prepared according to General Methods 10 and 11 from Compound 201F and Compound 203-5A P2. Data for Compound 203 P2: MS (ESI) m/z 766.2 (M+H)⁺; t_{R} 4.19 min (10% CH₃CN/H₂O−90% CH₃CN/H₂O, 6.5 min, 1.0 mL/min Gemini-NX C18, 4.6×50 mm).

### Example 38

### Preparation of Compound 205 P1

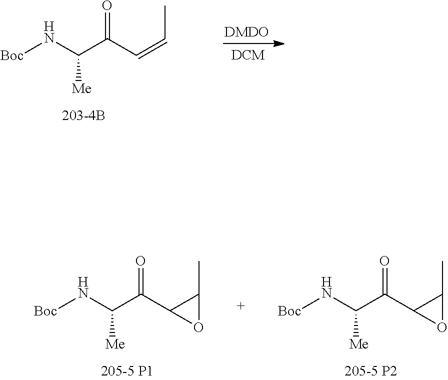 [SMILES: C/C=C\C(=O)[C@H](C)NC(=O)OC(C)(C)C ; CC1OC1C(=O)[C@H](C)NC(=O)OC(C)(C)C ; CC1OC1C(=O)[C@H](C)NC(=O)OC(C)(C)C]

DMDO was prepared from 60 g oxone, 58 g NaHCO₃ in 192 mL acetone and 254 mL water according to Chem. Ber. 1991, 124, 2377 and is herein incorporated by reference. DMDO solution (57 mL) was added to a solution of 203-4B (300 mg, 1.41 mmol) in 10 mL DCM at 0° C. The reaction was stirred at room temperature for 24 h. The solvent was evaporated under reduced pressure to afford an oil. Flash chromatography (10% EtOAc/hexanes-40% EtOAc/hexanes) afforded 95 mg (29%) of Compound 205-5 P1 and 100 mg (31%) of Compound 205-5 P2. Data for Compound 205-5 P1: ¹H NMR (CDCl₃, 500 MHz) δ 5.16 (broad s, 1H), 4.5-4.6 (m, 1H), 3.68 (d, J=4.5, 1H), 3.38 (app quintet, J=5.5, 1H), 1.44 (s, 9H), 1.36 (d, J=7.5, 3H), 1.31 (d, J=5.5, 3H). R_{f} 0.48 (2:1 hexanes:EtOAc). Data for Compound 205-5 P2: ¹H NMR (CDCl₃, 500 MHz) δ 5.08 (broad s, 1H), 4.45-4.55 (m, 1H), 3.84 (d, J=4.5, 1H), 3.40 (app quintet, J=5, 1H), 1.44 (s, 9H), 1.40 (d, J=7.0, 3H), 1.27 (d, J=5.5, 3H). R_{f} 0.41 (2:1 hexanes:EtOAc).

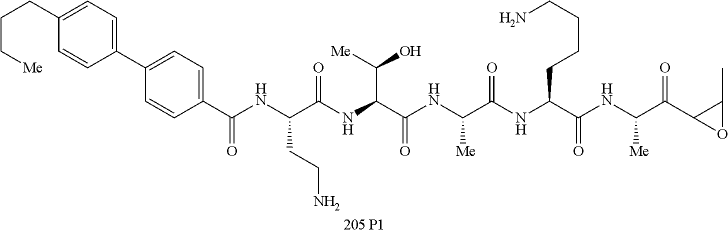 [SMILES: CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)C3OC3C)[C@@H](C)O)cc2)cc1]

Compound 205 P1 was prepared according to General Methods 10 and 11 from Compound 201F and Compound 205-5 P1. Data for Compound 205 P1: MS (ESI) m/z 766.2 (M+H)⁺; t_{R} 4.13 min (10% CH₃CN/H₂O−90% CH₃CN/H₂O, 6.5 min, 1.0 mL/min Gemini-NX C18, 4.6×50 mm).

### Example 39

### Preparation of Compound 205 P2

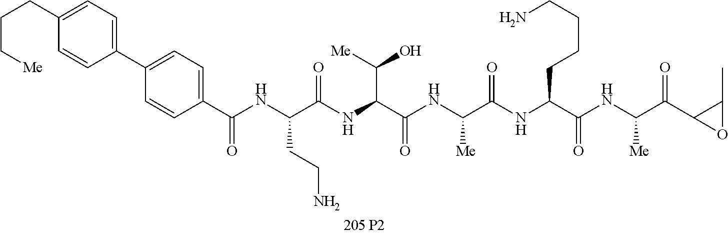 [SMILES: CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)C3OC3C)[C@@H](C)O)cc2)cc1]

Compound 205 P2 was prepared according to General Methods 10 and 11 from Compound 201F and Compound 205-5 P2. Data for Compound 205 P2: MS (ESI) m/z 766.3 (M+H)⁺; t_{R} 4.13 min (10% CH₃CN/H₂O−90% CH₃CN/H₂O, 6.5 min, 1.0 mL/min Gemini-NX C18, 4.6×50 mm).

### Example 40

### Preparation of Compound 207 P1

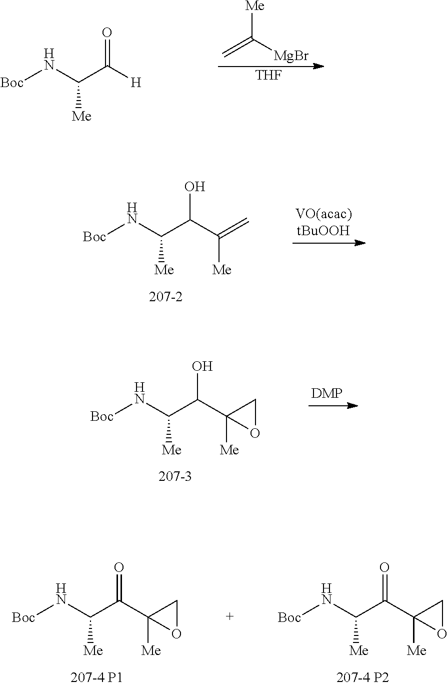

To a solution of L-Boc-Alaninal (100 mg, 0.58 mmol, 1 eq) in THF (4 mL) at −78° C. was added a 0.5 M solution of isopropenyl magnesium bromide in THF (3.5 mL, 3 eq) dropwise over 10 min with stirring. The solution was then allowed to warm to room temperature, and after 2 hrs TLC indicated complete consumption of starting material. The solution was then cooled to 0° C., 2 mL of 1 N HCl was added and the majority of the THF was removed via rotary evaporator. Additional 1 N HCl was added, the aqueous layer was extracted 3 times with DCM, then the combined organic layers were washed twice with water, once with brine, dried over sodium sulfate and concentrated. The crude material was purified via flash chromatography (0 to 50% EtOAc in hexanes) to give Compound 207-2 as a mixture of diastereomers (38% yield, R_{f}˜0.6 (30% EtOAc in hexanes)).

To a solution of Compound 207-2 (46 mg, 0.23 mmol, 1 eq) in anhydrous DCM (2 mL) under Ar was added vanadyl acetoacetonate (3 mg, 0.05 eq) and the solution was allowed to stir for 5 mins. A 5.5 M solution of t-BuOOH in decane (84 uL, 2 eq) was added dropwise then the solution was allowed to stir under Ar for 5 hrs. The reaction mixture was then filtered through celite, diluted with DCM and washed twice with aqueous NaHCO₃ (approximately 0.5 M). The combined aqueous layers were extracted twice with DCM then the combined organic layers were washed with water and brine, dried over sodium sulfate and concentrated to give an oil containing a crude mixture of products comprising Compound 207-3 as a mixture of diastereomers (R_{f} 0.45 (25% EtOAc in hexanes)) and residual decane.

To a solution of Compound 207-3 (90 mg, 0.39 mmol, 1 eq) in anhydrous DCM (2 mL) under Ar at 4° C. was added a suspension of Dess-Martin periodinane (413 mg, 2.5 eq) in anhydrous DCM (2 mL). The mixture was allowed to warm to room temperature and stirred for 4 hrs. To the reaction was then added saturated NaHCO₃ and the aqueous layer was extracted 3 times with EtOAc. The combined organics were washed twice with water, then brine, dried over sodium sulfate and concentrated. The crude material was purified by flash chromatography to give Compound 207-4 P1 (18 mg, R_{f} 0.8 (25% EtOAc in hexanes) and Compound 207-4 P2 (8 mg, R_{f} 0.7 (25% EtOAc in hexanes)) (29% combined yield). Data for Compound 207-4 P1: ¹H NMR (CDCl₃, 500 MHz) δ 5.05-4.95 (m, 1H), 4.34-4.28 (m, 1H), 3.23 (d, J=4.5 Hz, 1H), 2.89 (d, J=4.5 Hz, 1H), 1.52 (s, 3H), 1.41 (s, 9H), 1.31 (d, J=7 Hz, 3H). Data for Compound 207-4 P2: ¹H NMR (CDCl₃, 500 MHz) δ 5.15-4.95 (m, 1H), 4.63-4.50 (m, 1H), 3.03 (d, J=5 Hz, 1H), 2.86 (d, J=5 Hz, 1H), 1.56 (s, 3H), 1.43 (s, 9H), 1.25 (d, J=7 Hz, 3H).

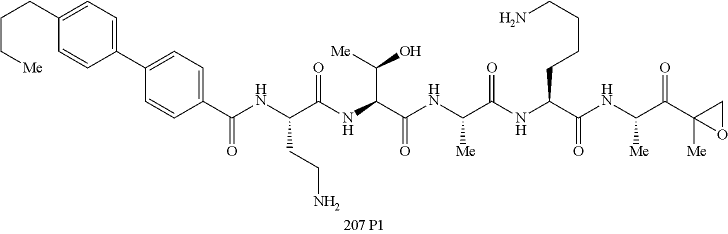 [SMILES: CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)C3(C)CO3)[C@@H](C)O)cc2)cc1]

Compound 207 P1 was prepared according to General Methods 10 and 11 (Example 34) from Compound 101F and Compound 107-4 P1. Data for Compound 107 P1: MS (ESI) m/z 766.4 (M+H)⁺; t_{R} 4.20 min (10% CH₃CN/H₂O−90% CH₃CN/H₂O, 6.5 min, 1.0 mL/min Gemini-NX C18, 4.6×50 mm).

### Example 41

### Preparation of Compound 207 P2

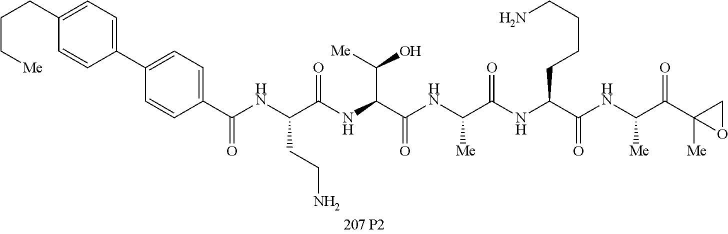 [SMILES: CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)C3(C)CO3)[C@@H](C)O)cc2)cc1]

Compound 207 P2 was prepared according to General Methods 10 and 11 (Example 34) from Compound 101F and Compound 107-4 P2. Data for Compound 207 P2: MS (ESI) m/z 766.2 (M+H)⁺; t_{R} 4.17 min (10% CH₃CN/H₂O−90% CH₃CN/H₂O, 6.5 min, 1.0 mL/min Gemini-NX C18, 4.6×50 mm).

### Example 42

### Preparation of Compound 209

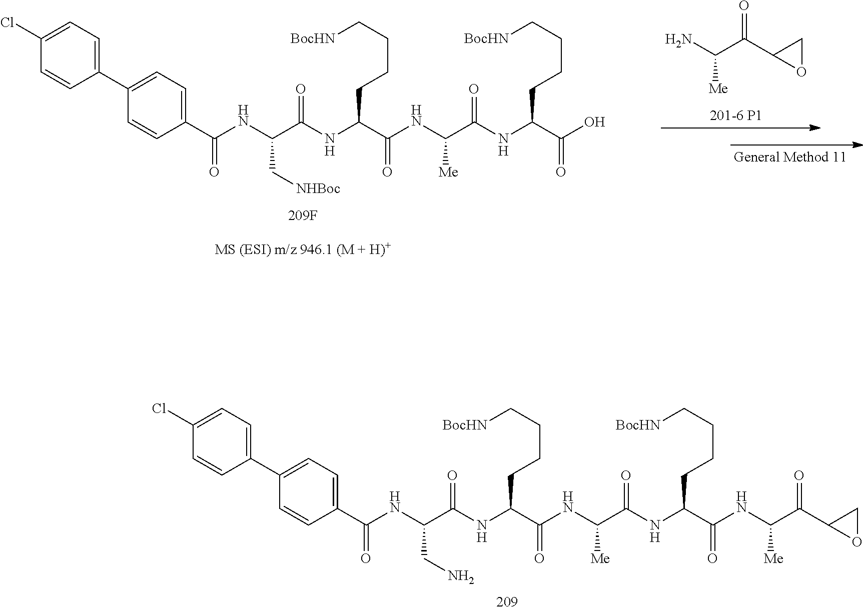 [SMILES: C[C@H](NC(=O)[C@H](CCCCNC(=O)OC(C)(C)C)NC(=O)[C@H](CNC(=O)OC(C)(C)C)NC(=O)c1ccc(-c2ccc(Cl)cc2)cc1)C(=O)N[C@@H](CCCCNC(=O)OC(C)(C)C)C(=O)O ; C[C@H](N)C(=O)C1CO1 ; C[C@H](NC(=O)[C@H](CCCCNC(=O)OC(C)(C)C)NC(=O)[C@H](CN)NC(=O)c1ccc(-c2ccc(Cl)cc2)cc1)C(=O)N[C@@H](CCCCNC(=O)OC(C)(C)C)C(=O)N[C@@H](C)C(=O)C1CO1]

Compound 209F was prepared utilizing the methods described for Compound 201F (General Method 9).

Compound 209 was prepared according to General Methods 10 and 11 from Compound 209F and Compound 201-6 P1. Data for Compound 209: MS (ESI) m/z 743.3 (M+H)⁺; t_{R} 3.15 min (10% CH₃CN/H₂O−90% CH₃CN/H₂O, 6.5 min, 1.0 mL/min Gemini-NX C18, 4.6×50 mm).

### Example 43

### Preparation of Compound 210

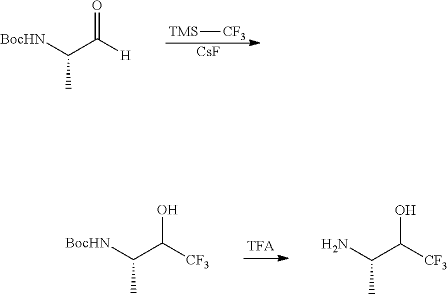

To a solution of Boc-L-alaninal (173 mg g, 1.0 mmol) in anhydrous THF (2.0 mL) at 0° C. under a nitrogen atmosphere was slowly added (trifluoromethyl)-trimethylsilane (2.0 M solution in THF, 1.0 mL, 2.0 mmol) and the mixture was stirred at 0° C. for 30 minutes, and then cesium fluoride (228 mg, 1.5 mmol) was slowly added portions wise and the reaction was allowed to stir at room temperature overnight. The reaction mixture was then poured into water and extracted with EtOAc (3×15 mL). The combined organic layers were washed with brine, dried (Na₂SO₄), filtered, and concentrated. The resulting residue was purified by flash chromatography (0-50% EtOAc/hexanes) to isolate tert-butyl (2S)-4,4,4-trifluoro-3-hydroxybutan-2-ylcarbamate, a diastereomeric mixture of products as light brown oil (68 mg, 26%). MS (ESI) m/z 144 (M+H-Boc)⁺. The diastereomeric mixture was dissolved in 1:4 TFA-DCM (1 mL) and stirred at room temperature for 1 h monitoring the reaction by TLC. After completion of the reaction, the solvents are removed and dried under high vacuum to afford (3S)-3-amino-1,1,1-trifluorobutan-2-ol as its TFA salt.

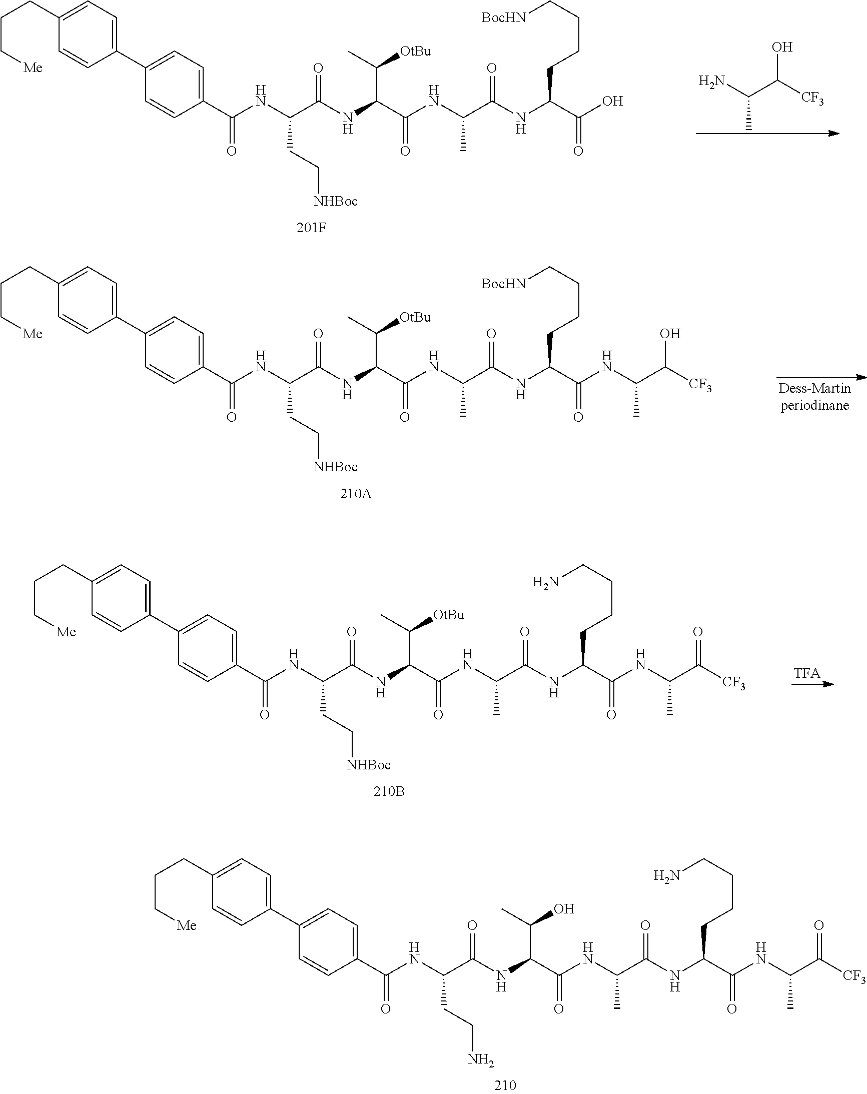

To a solution of Compound 201F (25 mg, 0.025 mmol) in anhydrous DMF (1 mL) under nitrogen atmosphere was added HATU (20 mg, 0.05 mmol), DIEA (18 μL, 0.1 mmol) and (3S)-3-amino-1,1,1-trifluorobutan-2-ol (36 mg, 0.25 mmol). The reaction mixture was stirred at room temperature overnight. Water was added and extracted with EtOAc (3×20 mL). The combined organic layers were washed with brine, dried (Na₂SO₄), filtered, and concentrated. The resulting residue was purified by flash chromatography (1:4 MeOH-DCM and DCM) to isolate Compound 210A as light brown oil 20 mg (77%). MS (ESI) m/z 1036 (M+H)⁺.

To a solution of Compound 210A (20 mg, 0.02 mmol) in anhydrous DCM (2 mL) was added Dess-Martin periodinane (26 mg, 0.06 mmol) in one portion at 0° C. The reaction mixture was allowed to stir at room temperature overnight. After LCMS showed the reaction was complete, the mixture was filtered through celite and the filtrate was washed sequentially with saturated Na₂SO₃ solution, saturated NaHCO₃ solution and brine. The organic layer was dried over Na₂SO₄, filtered, concentrated to afford Compound 210B. MS (ESI) m/z 1034 (M+H)⁺.

Compound 210B was dissolved in 1:4 TFA-DCM (1 mL) and stirred at room temperature for 1 h. After ELSD showed the reaction was complete, the solvent was removed. The residue was purified by prep-HPLC (CH₃CN/H₂O in 0.05% TFA) to afford 7.0 mg (50%) of Compound 210 as a white solid. MS (ESI) for (C₃₈H₅₅F₃N₇O₇): 778 m/z (M+H)⁺. Observed; 796 m/z (M+H₂O+H)⁺.

### Example 44

### Preparation of Compound 211

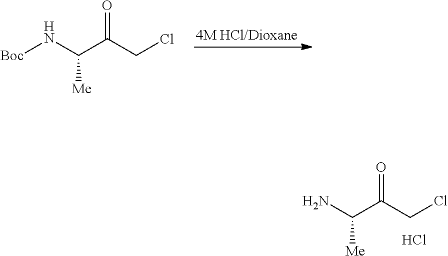 [SMILES: C[C@H](NC(=O)OC(C)(C)C)C(=O)CCl ; C[C@H](N)C(=O)CCl ; Cl]

To a solution of (S)-3-(Boc-amino)-1-chloro-2-butanone (232 mg, 0.23 mmol, 1 eq) in dioxane at 0° C. under Ar was added 4 M HCl in dioxane (2.6 mL, 10 eq). The reaction was allowed to warm to room temperature, then stirred for 2.5 hrs at which time TLC indicated complete consumption of starting materials. The volatiles were evaporated, then the residue was azeotroped with MeOH and dioxane and dried under vacuum to afford (S)-3-amino-1-chloro-2-butanone hydrochloride.

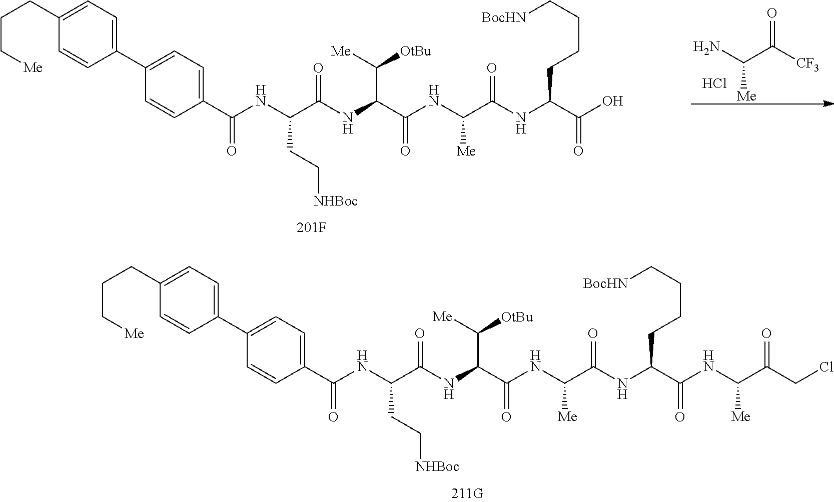 [SMILES: CCCCc1ccc(-c2ccc(C(=O)N[C@@H](CCNC(=O)OC(C)(C)C)C(=O)N[C@H](C(=O)N[C@@H](C)C(=O)N[C@@H](CCCCNC(=O)OC(C)(C)C)C(=O)O)[C@@H](C)OC(C)(C)C)cc2)cc1 ; Cl ; C[C@H](N)C(=O)C(F)(F)F ; CCCCc1ccc(-c2ccc(C(=O)N[C@@H](CCNC(=O)OC(C)(C)C)C(=O)N[C@H](C(=O)N[C@@H](C)C(=O)N[C@@H](CCCCNC(=O)OC(C)(C)C)C(=O)N[C@@H](C)C(=O)CCl)[C@@H](C)OC(C)(C)C)cc2)cc1]

To a solution of Compound 201F (175 mg, 0.19 mmol, 1 eq) in anhydrous DMF under Ar at 0° C. was added (S)-3-amino-1-chloro-2-butanone hydrochloride (60 mg, 2 eq), HATU (144 mg, 2 eq) then N-methylmorpholine (83 uL, 4 eq). After 1 hr LCMS indicated complete conversion of the starting material and water was added. The aqueous layer was extracted 3 times with EtOAc, the combined organic layers were washed with half saturated aqueous NaCl, dried over sodium sulfate and concentrated. The crude material was purified by flash chromatography (0 to 9% MeOH in DCM) to give the Compound 211G (95% yield, R_{f} 0.65 in 8% MeOH in DCM). MS (ESI) m/z 1014.5 (M+H)⁺; t_{R} 7.28 min (50% CH₃CN/H₂O−95% CH₃CN/H₂O, 7 min, then 95% CH₃CN/H₂O, 0.5 min, 1.0 mL/min Gemini-NX C18, 4.6×50 mm).

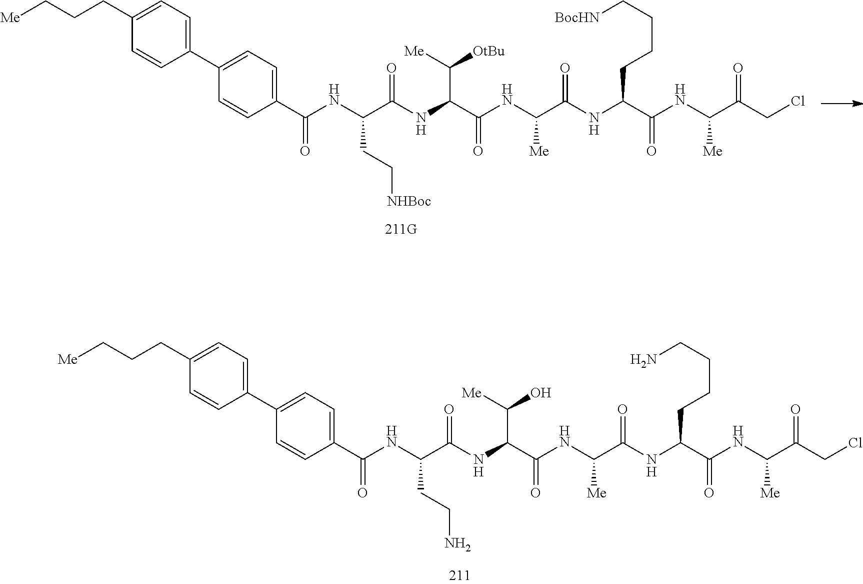 [SMILES: CCCCc1ccc(-c2ccc(C(=O)N[C@@H](CCNC(=O)OC(C)(C)C)C(=O)N[C@H](C(=O)N[C@@H](C)C(=O)N[C@@H](CCCCNC(=O)OC(C)(C)C)C(=O)N[C@@H](C)C(=O)CCl)[C@@H](C)OC(C)(C)C)cc2)cc1 ; CCCCc1ccc(-c2ccc(C(=O)N[C@@H](CCN)C(=O)N[C@H](C(=O)N[C@@H](C)C(=O)N[C@@H](CCCCN)C(=O)N[C@@H](C)C(=O)CCl)[C@@H](C)O)cc2)cc1]

Compound 211G (9.4 mg, 0.009 mmol, 1 eq) was treated with a 4:1 mixture of DCM:TFA at 0° C. The solution was stirred until LCMS analysis showed complete conversion to the product (˜2 hrs) then the solvents were evaporated and the residue was azeotroped twice with DCM and dried under vacuum. The crude residue was taken up in MeOH, centrifuged to remove insoluble particulates and purified via HPLC to give Compound 211 (19% yield). MS (ESI) m/z 758.4 (M+H)⁺; t_{R} 4.22 min (10% CH₃CN/H₂O−90% CH₃CN/H₂O, 6.5 min, 1.0 mL/min Gemini-NX C18, 4.6×50 mm).

### Example 45

### Preparation of Compound 212

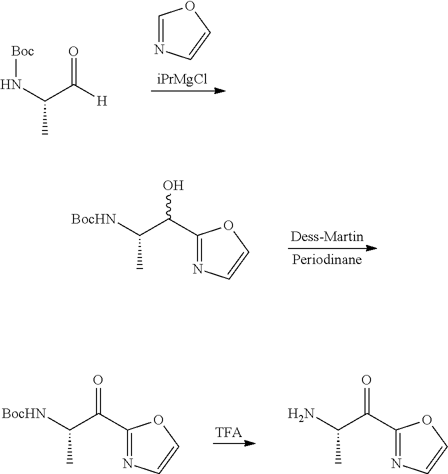

To a solution of oxazole (329 mg, 4.76 mmol) in toluene (5 mL), isopropyl magnesium chloride (2M in THF solution, 2.38 mL, 4.76 mmol) was added at 0° C. and stirred for 1 h. The resulting mixture was added to a solution of (S)-tert-butyl (1-oxopropan-2-yl) carbamate (750 mg, 4.33 mmol) in THF (10 mL) at 0° C. and stirred for 1 hour then at room temperature for 3 hours. The reaction mixture was quenched with 5% sodium carbonate (10 mL), extracted by ethyl acetate (30 mL×3). The combined organic layer was washed with brine (10 mL×3), dried over Na₂SO₄ and then the mixture was filtered. The filtrate was concentrated under reduced pressure to give the crude product, which was purified by silica gel column (eluting with 5% methanol in dichloromethane) to give (5)-tert-butyl 1-hydroxy-1-(oxazol-2-yl)propan-2-ylcarbamate (672 mg, 64%) as a yellow oil. ¹H NMR (400 MHz, CDCl₃) δ 1.10 (1.5H, d, J=6.8 Hz), 1.12 (1.5H, d, J=6.8 Hz), 1.41 (6H, s), 1.45 (6H, s), 4.75 (0.5H, d, J=3.2 Hz), 4.87 (1H, br), 5.01 (0.5H, d, J=3.2 Hz), 7.90 (1H, d, J=12.4 Hz), 7.67 (s, 1H).

To a solution (S)-tert-butyl 1-hydroxy-1-(oxazol-2-yl)propan-2-ylcarbamate (672 mg, 2.77 mmol) in anhydrous dichloromethane (5 mL), Dess-Martin reagent (2.35 g, 5.55 mmol) was added at 0° C. The reaction was stirred at 0° C. for 1 hour and then room temperature overnight. The reaction mixture was diluted with ethyl acetate (100 mL), washed with NaOH (1M, 10 mL×3), brine (20 mL×3) and dried over Na₂SO₄. Crude product was obtained after filtration and concentration, which was further purified by silica gel chromatography (eluting with 5% methanol in dichloromethane) to give (S)-tert-butyl 1-(oxazol-2-yl)-1-oxopropan-2-ylcarbamate (555.7 mg, 83.4%) as a white solid. ¹H NMR (400 MHz, CDCl₃) δ 1.44 (s, 9H), 1.52 (3H, d, J=6.8 Hz), 4.12 (1H, q, J=6.8 Hz), 7.38 (s, 1H), 7.86 (s, 1H).

To a solution of (S)-tert-butyl 1-(oxazol-2-yl)-1-oxopropan-2-ylcarbamate (212 mg, 0.88 mmol) in dichloromethane (6 mL), was added TFA (2 mL). The reaction mixture was stirred at room temperature for 3 hours and concentrated under reduced pressure to give the crude product(S)-2-amino-1-(oxazol-2-yl)propan-1-one, which was used in next step without further purification.

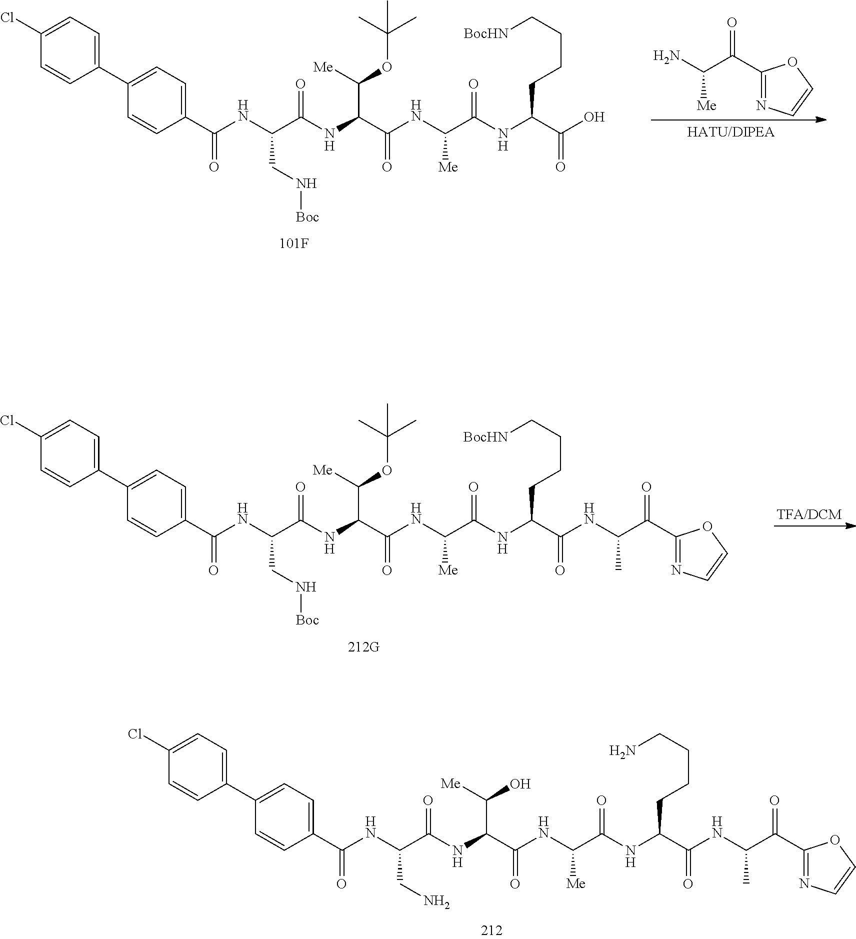 [SMILES: C[C@H](NC(=O)[C@@H](NC(=O)[C@H](CNC(=O)OC(C)(C)C)NC(=O)c1ccc(-c2ccc(Cl)cc2)cc1)[C@@H](C)OC(C)(C)C)C(=O)N[C@@H](CCCCNC(=O)OC(C)(C)C)C(=O)O ; C[C@H](N)C(=O)c1ncco1 ; C[C@H](NC(=O)[C@@H](NC(=O)[C@H](CNC(=O)OC(C)(C)C)NC(=O)c1ccc(-c2ccc(Cl)cc2)cc1)[C@@H](C)OC(C)(C)C)C(=O)N[C@@H](CCCCNC(=O)OC(C)(C)C)C(=O)N[C@@H](C)C(=O)c1ncco1 ; C[C@H](NC(=O)[C@@H](NC(=O)[C@H](CN)NC(=O)c1ccc(-c2ccc(Cl)cc2)cc1)[C@@H](C)O)C(=O)N[C@@H](CCCCN)C(=O)N[C@@H](C)C(=O)c1ncco1]

A solution of Compound 101F (103 mg, 0.117 mmol, prepared as in Example 1) in anhydrous DMF (3 mL) was treated with (S)-2-amino-1-(oxazol-2-yl)propan-1-one followed by DIEA (60.8 mg, 0.47 mmol) and HATU (100 mg, 0.228 mmol). The mixture was stirred at room temperature overnight, then poured into ice-water (10 mL), and filtered to give the crude product of Compound 212G (85.25 mg, yield 74.6%) as a yellowish solid. To a solution of compound 212G (85.25 mg, 0.085 mmol) in dichloromethane (3 mL) was added TFA (1 mL) and the mixture was stirred at room temperature for 3 hours. The mixture was concentrated to give the crude product, which was purified by prep-HPLC to give Compound 212 (58.2 mg, 92% yield) as a white solid. MS (ESI) for (C₃₅H₄₅ClN₈O₈): m/z 741.3 (M+H). ¹H NMR (400 MHz, CD₃OD) δ 1.26-1.28 (3H, m), 1.42-1.55 (8H, m), 1.70-1.78 (4H, m), 1.87-1.95 (1H, m), 2.97-3.02 (2H, m), 3.36-3.37 (2H, m), 3.43-3.48 (1H, m), 3.56-3.60 (1H, m), 4.30-4.50 (5H, m), 5.34-5.40 (1H, m), 7.51-7.54 (3H, m), 7.72 (2H, d, J=8 Hz), 7.80 (2H, m), 8.07 (2H, d, J=8.0 Hz), 8.20 (2H, d, J=7.3 Hz), 8.57 (s, br, 2H).

### Example 46

### Preparation of Compound 213

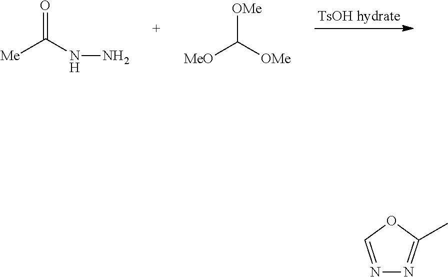

To a mixture of acethydrazide (7.4 g, 100 mmol) and trimethylorthoformate (54.6 mL, 500 mmol) was added TsOH hydrate (1.9 g, 10 mmol). The solution was heated to reflux for 24 h, and then the mixture was concentrated to an oil. Flash chromatography (1% MeOH/DCM to 12% MeOH/DCM) afforded 0.63 g (15%) of 2-methyl-1,3,4-oxadiazole. ¹H NMR (CDCl₃, 500 MHz) δ 8.32 (s, 1H), 2.57 (s, 3H).

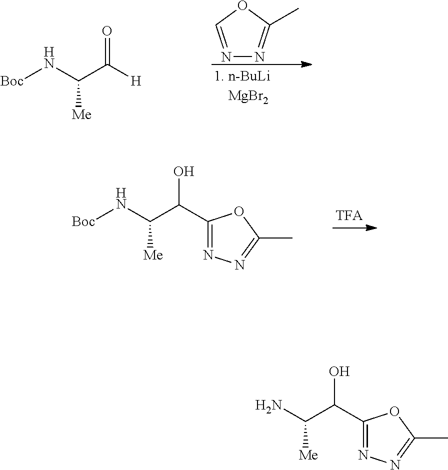

In a flame-dried, round bottom flask, a solution of 2-methyl-1,3,4-oxadiazole (0.37 g, 4.4 mmol) in 17.6 mL anhydrous THF was cooled to −78° C. n-BuLi (2.0 M in cyclohexanes, 2.2 mL, 4.4 mmol) was added dropwise and the mixture was stirred at −78° C. for 2 hr, whereupon MgBr₂ etherate (1.14 g, 4.4 mmol) was added in one portion. The yellow suspension was stirred for 1.5 hr, then a solution of Boc-L-alaninal (0.35 g, 2.0 mmol) in 5 mL THF was added dropwise at −78° C. After 1 hr, the solution was allowed to warm to −20° C., and kept between −20° C. and −25° C. for 1.5 hr. The solution was poured into a mixture of cold ether/0.2 N NaHSO₄, and the aqueous layer was extracted with ether. The combined organic layers were washed sequentially with saturated NaHCO₃ and brine, dried over Na₂SO₄, filtered, and concentrated. Flash chromatography (100% DCM to 12% MeOH/DCM gave tert-butyl (2S)-1-hydroxy-1-(5-methyl-1,3,4-oxadiazol-2-yl)propan-2-ylcarbamate (98 mg, 10%). MS (ESI) m/z 157 (M−Boc+H)⁺.

A solution of tert-butyl (2S)-1-hydroxy-1-(5-methyl-1,3,4-oxadiazol-2-yl)propan-2-ylcarbamate (96 mg, 0.37 mmol) in 2 mL DCM is cooled to 0° C. TFA (0.4 mL) was added and the solution was warmed to room temperature and stirred for 15 min. Volatiles were removed under reduced pressure, and dichloroethane was added and evaporated to remove residual TFA to afford (2S)-2-amino-1-(5-methyl-1,3,4-oxadiazol-2-yl)propan-1-ol as its trifluoracetate salt, which was carried on without further purification.

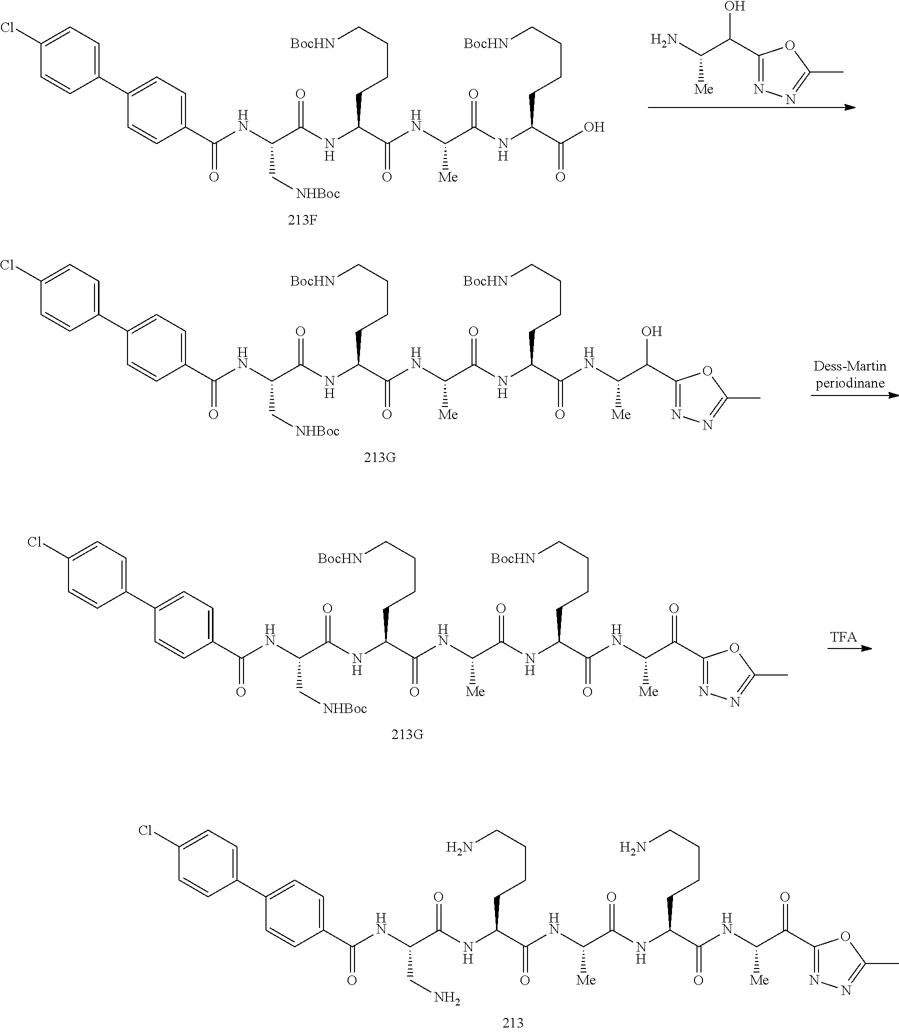

Compound 213F was prepared utilizing the methods described for compound 101F (General Method 1, Example 1). Data for compound 213F: MS (ESI) m/z 946.1 (M+H)⁺.

To a solution of compound 213F (61 mg, 0.064 mmol), (25)-2-amino-1-(5-methyl-1,3,4-oxadiazol-2-yl)propan-1-ol (23 mg, 0.084 mmol), and HATU (50 mg, 0.13 mmol) in 0.8 mL DMF and 0.8 mL DCM at 0° C. was added DIPEA (33 mg. 0.25 mmol). The solution was allowed to warm to room temperature and was stirred for 1.5 hr. The reaction mixture was partitioned between EtOAc and 0.1 N NaHSO₄, and the aqueous layer was extracted with EtOAc. The organic layers were washed sequentially with saturated NaHCO₃ and brine, dried over Na₂SO₄, filtered, and concentrated. Flash chromatography (1% MeOH/DCM to 12% MeOH/DCM) afforded 41 mg (59%) of compound 213G. MS (ESI) m/z 1085.5 (M+H)⁺.

Dess-Martin periodinane (47 mg, 0.11 mmol) was added to a solution of Compound 213G (40 mg, 0.037 mmol) in 4 mL DCM at room temperature. After 3 hr, a few drops of MeOH was added, and the mixture was filtered through Celite. The filtrate was partitioned between saturated NaHCO₃ and DCM, and the aqueous layer was extracted with DCM. The combined organic layers were washed sequentially with aqueous sodium sulfite and brine, dried over Na₂SO₄, filtered and concentrated. Flash chromatography (1% MeOH/DCM to 12% MeOH/DCM) afforded 34 mg (85%) of Compound 213H. MS (ESI) m/z 1083.4 (M+H)⁺.

To a solution of Compound 213H (33 mg, 0.03 mmol) in 2 mL DCM cooled to 0° C. was added 0.5 mL TFA. The mixture was allowed to warm to room temperature and was stirred for 40 min. The TFA and DCM was evaporated under reduced pressure, and dichloroethane was added and removed under reduced pressure to remove residual TFA. The resultant oil was dissolved in 2 mL water and the compound was lyophilized to a solid to afford 22 mg (65%) of Compound 213 as the tris-trifluoroacetate salt. MS (ESI) m/z 783.3 (M+H)⁺; t_{R} 3.04 min (10% CH₃CN/H₂O−95% CH₃CN/H₂O, 6 min, 1.0 mL/min Gemini-NX C18, 4.6×50 mm).

### Example 47

### Preparation of Compound 214

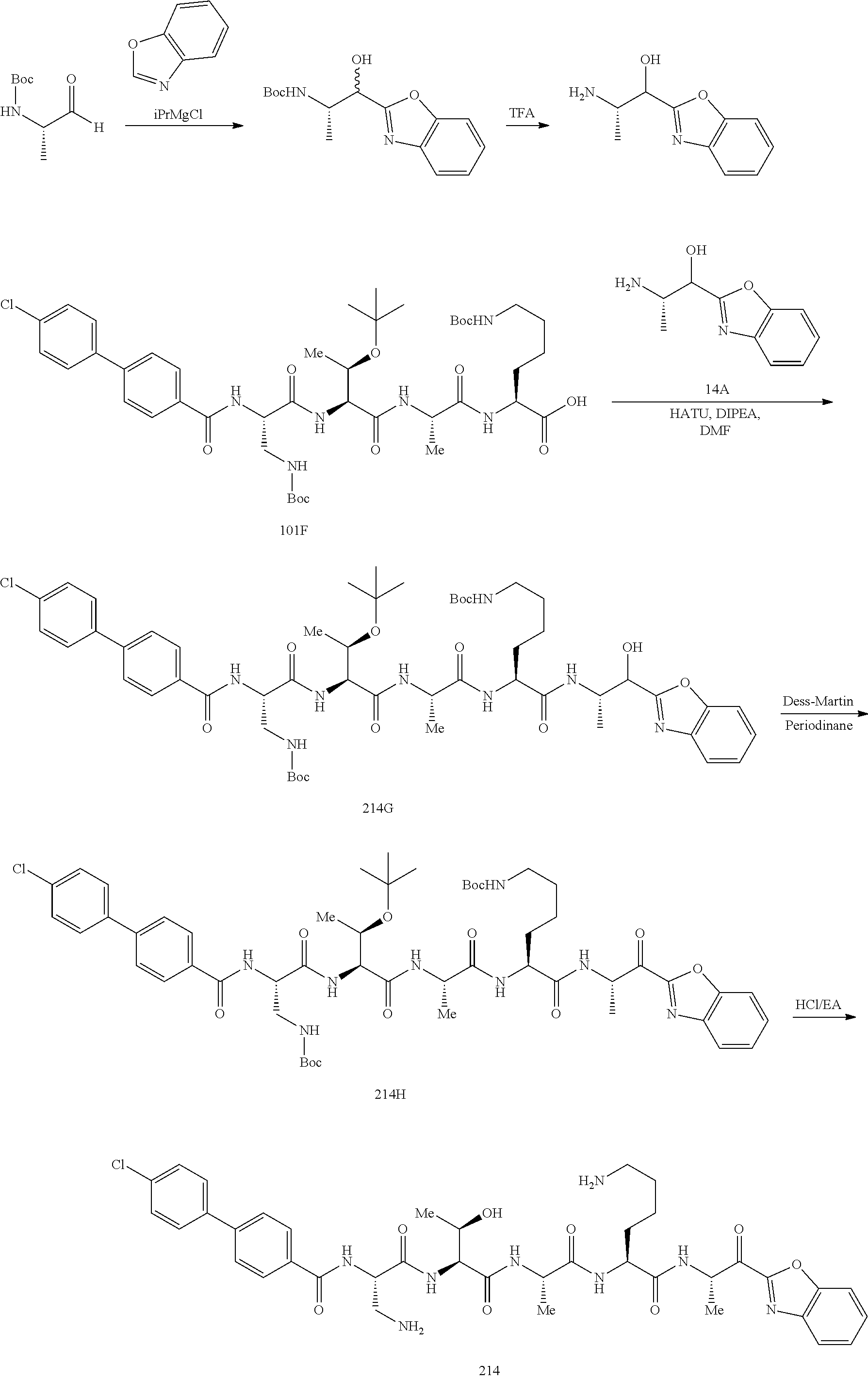

To a solution of benzoxazole (3.9 g, 7.51 mmol) in anhydrous toluene (5 mL), isopropyl magnesium chloride (2M in THF solution, 3.75 mL, 7.51 mmol) was added at 0° C. and stirred for 1 h. The resulting mixture was added to a solution of (S)-tert-butyl (1-oxopropan-2-yl) carbamate (1.0 g, 5.77 mmol) in THF (10 mL) at 0° C. and stirred for 4 h at room temperature. The reaction mixture was quenched with saturate NH₄Cl (20 mL) and extracted with ethyl acetate (50 mL×3). The combined organic layer was washed with brine (20 mL×3), dried over Na₂SO₄ and filtered. The filtrate was concentrated under reduced pressure and the residue was purified by silica gel column chromatography (eluting with 4% methanol in dichloromethane) to give (S)-tert-butyl 1-(benzo[d]oxazol-2-yl)-1-hydroxypropan-2-ylcarbamate (1.0 g, 59%) as a yellow oil.

To a solution of (S)-tert-butyl 1-(benzo[d]oxazol-2-yl)-1-hydroxypropan-2-ylcarbamate (300 mg, 1.03 mmol) in dichloromethane (5 mL) at 0° C., TFA (1.5 mL) was added at 0° C. and stirred at room temperature for 2 h. The reaction mixture was concentrated under reduced pressure to give the crude product (2S)-2-amino-1-(benzo[d]oxazol-2-yl)propan-1-ol (199 mg), which was used in next step without further purification.

A mixture of (2S)-2-amino-1-(benzo[d]oxazol-2-yl)propan-1-ol (199 mg, 1.03 mmol), Compound 101F (400 mg, 0.457 mmol) and DIPEA (177 mg, 1.37 mmol) in N,N-dimethylformamide (5 mL) was stirred at 0° C. for 5 min. Then HATU (347 mg, 0.914 mmol) was added and the mixture was stirred at room temperature for 3 h. The reaction mixture was poured into ice-water (80 mL) and filtered. The cake was washed with water (40 mL*3) and dried under reduced pressure to give the crude desired compound, which was purified by silica gel column chromatography (eluting with 2% to 6% methanol in dichloromethane) to give Compound 214G (270 mg, 56% yield) as a mixture of diastereomers in the form of a white solid.

To a solution of Compound 214G (270 mg, 0.26 mmol) in anhydrous dichloromethane (8 mL), Dess-Martin reagent (218 mg, 0.514 mmol) was added at 0° C. The reaction was stirred at 0° C. for 1 hour and room temperature overnight. The reaction mixture was diluted with dichloromethane (100 mL), washed with Na₂S₂O₃ (2M, 10 mL×3), NaOH (1M, 10 mL×3), brine (20 mL×3) and dried over Na₂SO₄. Crude product was obtained after filtration and concentration, which was further purified by silica gel column (eluting with 5% methanol in dichloromethane) to give Compound 214H (245 mg, 91%) as a white solid.

A solution of Compound 214H (245 mg, 0.233 mmol) in HCl/EtOAc (4M, 6 mL) was stirred at room temperature for 3 hours. The mixture was concentrated to give the crude product, which was purified by prep-HPLC to give Compound 214 (40 mg, 21.6% yield) as a white solid. MS (ESI) for (C₃₉H₄₇ClN₈O₈): m/z 791.0 (M+H). ¹H NMR (400 MHz, CD₃OD) δ 8.00 (d, J=7.6 Hz, 2H), 7.66-7.75 (m, 3H), 7.64-7.65 (m, 3H), 7.38-7.48 (m, 4H), 4.48-4.60 (m, 4H), 4.28-4.43 (m, 4H), 3.48-3.53 (m, 1H), 2.89-2.93 (m, 2H), 1.17-1.66 (m, 15H).

### Example 48

### Preparation of Compound 215

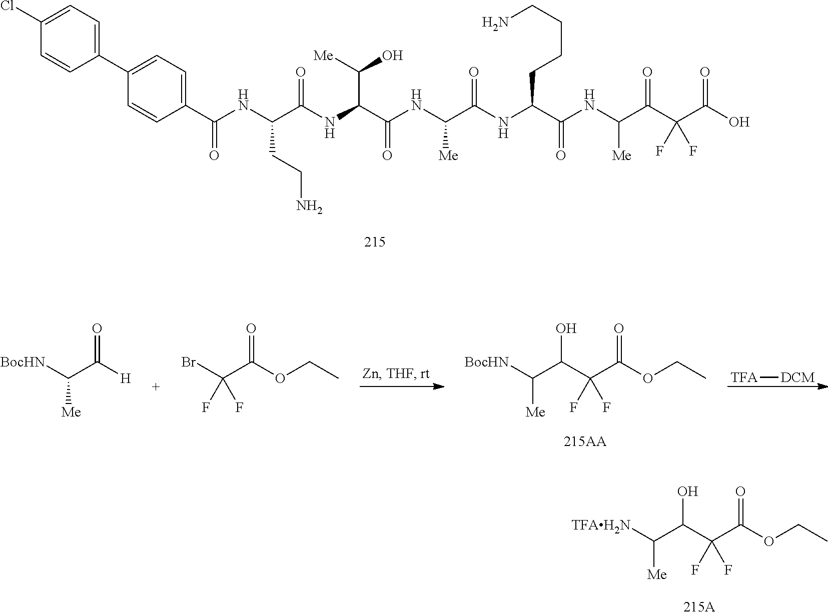

To a suspension of Zn dust (198 mg, 3.0 mmol) was added ethyl 2-bromo-2,2-difluoroacetate (400 μL, 2.5 mmol). The reaction mixture was stirred at rt for about 1 h and heated at 50° C. for about 10 min to initiate the reaction. (S)-tert-butyl (1-oxopropan-2-yl)carbamate (173 mg, 1.0 mmol) was then added and the reaction mixture was stirred at rt for 2 h. After completion of the reaction, the reaction mixture was cooled in an ice bath and quenched with saturated NH₄Cl solution (10 mL). The mixture was diluted with water and extracted with EtOAc. The combined organic layers were washed with brine and dried over anhydrous Na₂SO₄, filtered and the solvent was removed. The residue was purified by flash chromatography (50% EtOAC-hexanes) to afford 183 mg (61%) of compound 215AA as a viscous oil. MS (ESI) for (C₁₂H₂₁F₂NO₅): m/z 198.1 (M+H-Boc, two peaks, 1:1 mixture of β-hydroxyester isomers).

A solution of compound 215AA (60 mg, 0.2 mmol) in 1:3 TFA-DCM (2 mL) was stirred at rt for about 1 to 2 h. After completion of the reaction, the solvent was evaporated and the residue was dried under vacuum to afford compound 215A.

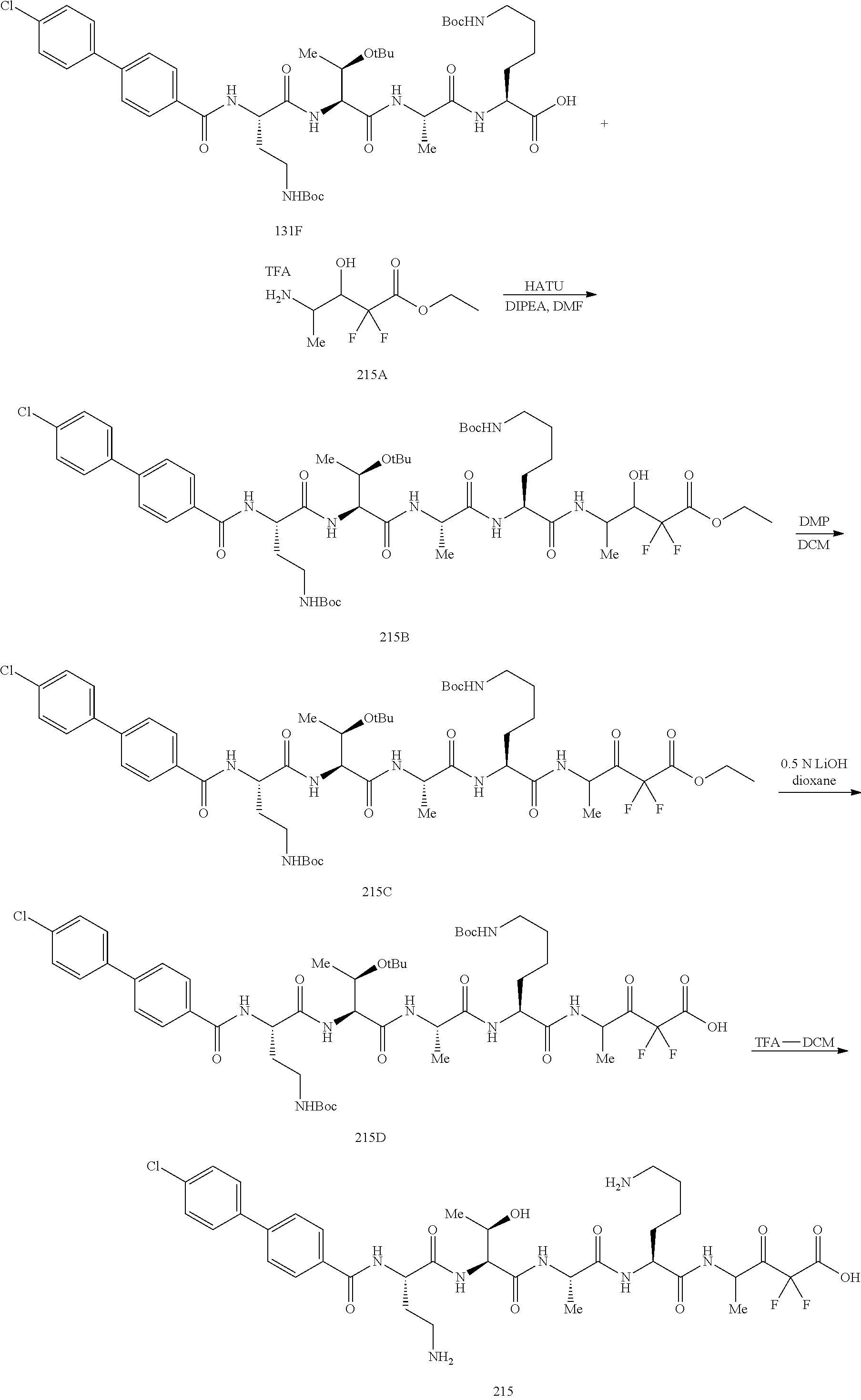

To a solution of compound 131F (45 mg, 0.05 mmol), compound 215A (30 mg, 0.1 mmol), and HATU (48 mg, 0.125 mmol) in DMF (1 mL) at 0° C. was added DIPEA (44 μL, 0.25 mmol). The solution was allowed to warm to rt and was stirred for 4 hr. The reaction mixture was partitioned between EtOAc and brine solution, and the aqueous layer was extracted with EtOAc. The organic layers were washed with brine, dried over Na₂SO₄, filtered, and concentrated. Flash chromatography (DCM to 20% MeOH/DCM) afforded 32 mg (69%) of compound 215B. MS (ESI) for (C₅₁H₇₆ClF₂N₇O₁₃); m/z 1068.4 (M+H)⁺ (mixture of diastereomers).

To a solution of compound 215B (32 mg, 0.03 mmol) in DCM (5 mL) was added Dess-Martin periodinane (63 mg, 0.15 mmol) and the heterogeneous mixture was stirred at rt overnight. The mixture was filtered through Celite and filtrate was partitioned between 1:1 mixture of saturated NaHCO₃—NaHSO₃ solution and DCM, and the aqueous layer was extracted with DCM. The combined organic layers were washed with brine, dried over Na₂SO₄, filtered and concentrated. Flash chromatography (DCM to 20% MeOH/DCM) afforded 20 mg (62%) of compound 215C. MS (ESI) for (C₅₁H₇₄ClF₂N₇O₁₃); m/z 1066.4 (M+H)⁺.

Compound 215C (20 mg, 0.02 mmol) was dissolved in dioxane-H₂O (3:1, 1 mL) and 0.5 M LiOH solution (60 μL, 0.03 mmol) was added at 0° C. The reaction mixture was stirred at rt for 3 h. After completion of the reaction, water (2 mL) was added and the mixture was acidified with 0.5 M HCl. The resultant white cloudy mixture was extracted with EtOAc. The combined organic layers were washed with brine, dried over anhydrous Na₂SO₄, filtered, and the solvent was removed. The residue was purified by prep HPLC (CH₃CN—H₂O containing 0.05% TFA) to afford 6 mg (30%) of compound 215D as a white solid. MS (ESI) for (C₄₉H₇₀ClF₂N₇O₁₃): m/z 1038.2 (M+H) broad peak).

A solution of compound 215D (6 mg, 0.005 mmol) in 1:3 TFA-DCM was stirred for about 2 h at rt. After completion of the reaction, solvent was removed and lyophilized to afford compound 215 as a trifluoroacetate salt. MS (ESI) for (C₃₅H₄₆ClF₂N₇O₉): m/z 782.2 (M+H) t_{R} 2.84 min (10% CH₃CN/H₂O−90% CH₃CN/H₂O, 3 min, 1.0 mL/min Kinetex-5u C18, 4.6×50 mm).

### Example 49

### Preparation of Compound 216

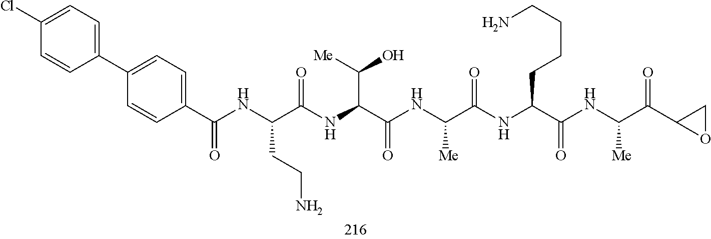 [SMILES: C[C@H](NC(=O)[C@@H](NC(=O)[C@H](CCN)NC(=O)c1ccc(-c2ccc(Cl)cc2)cc1)[C@@H](C)O)C(=O)N[C@@H](CCCCN)C(=O)N[C@@H](C)C(=O)C1CO1]

Compound 216 was prepared using General Methods 1 and 11 from compound 131F and compound 201-6 P1. MS (ESI) m/z 730.3 (M+H)⁺; t_{R} 3.72 min (10% CH₃CN/H₂O−90% CH₃CN/H₂O, 6.5 min, 1.0 mL/min Kinetex C18, 4.6×50 mm).

### Example 50

### Preparation of Compound 217

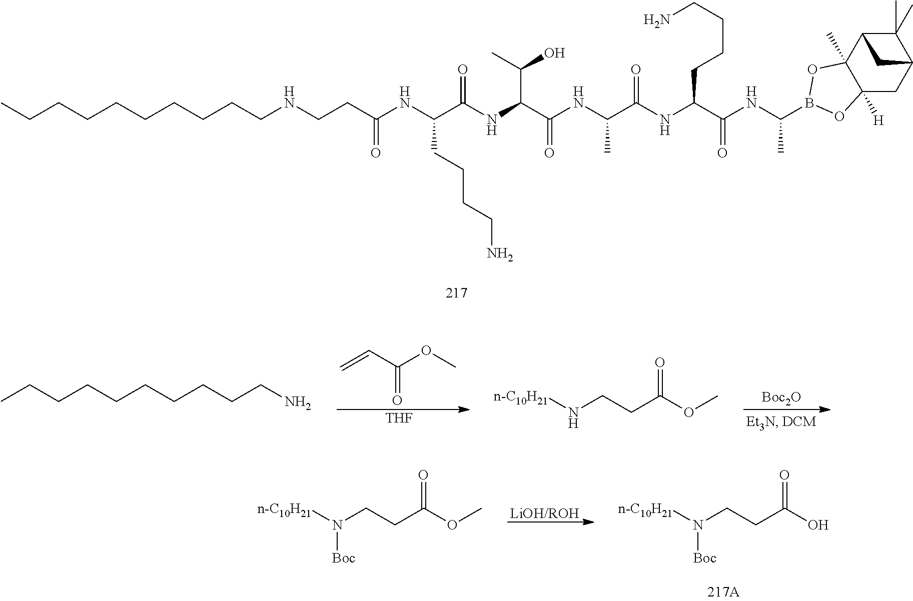

To a solution of methyl acrylate (2.2 g, 26 mmol) in THF (20 mL) was added a solution of decan-1-amine (6 g, 38 mmol) in THF (20 mL) at 0° C. The reaction mixture was stirred at 30° C. for 48 h. The resulting solution was concentrated to obtain methyl 3-(decylamino)propanoate (6.4 g).

To a solution of crude methyl 3-(decylamino)propanoate (6.4 g, 15 mmol) and Et₃N (4 g, 40 mmol) in DCM (30 mL) was added dropwise a solution of Boc₂O (5.7 g, 26 mmol) in DCM (20 mL) at 0° C. The reaction mixture was then allowed to warm to 30° C. gradually and stirred for 18 h. After the reaction was completed, H₂O (50 mL) was added and the resulting aqueous layer was further extracted with DCM (50 mL*2). The combined organic layers were concentrated and the residue was purified by silica gel column (PE/EtOAc=50/1˜20/1) to give methyl 3-((tert-butoxycarbonyl)(decyl)amino)propanoate (6.5 g, 73%) as a colorless oil.

To a solution of methyl 3-((tert-butoxycarbonyl)(decyl)amino)propanoate (8.2 g, 23.9 mmol, crude) in EtOH (40 mL) was added a solution of LiOH (1.15 g, 48 mmol) in H₂O (20 mL) at 0° C. The reaction mixture was then allowed to warm to 30° C. gradually and stirred for 18 h. After the reaction was complete, EtOH was removed under reduced pressure. The remaining aqueous solution was then adjusted to pH=2˜3 with 6 N HCl, followed by the extraction with EtOAc (50 mL*3). The combined EtOAc layers were dried over Na₂SO₄, and concentrated to give compound 217A (7 g, 88.6%) as a colorless oil. ¹H NMR (400 MHz, CDCl₃) δ 3.47-3.43 (t, J=6.8 Hz, 2H), 3.19-3.15 (t, J=7.2 Hz, 2H), 2.61 (brs, 2H), 1.51-1.39 (m, 11H), 1.24-1.22 (m, 14H), 0.88-0.84 (t, J=6.8 Hz, 3H).

General Method 12:

The LC-MS conditions for selected final compounds are the following using acetonitrile (0.02% TFA) and H₂O (0.04% TFA). HPLC 5% CH₃CN/H₂O−95% CH₃CN/H₂O, 0.7 min, then 95% CH₃CN/H₂O, 0.4 min; 1.5 mL/min, MERCK RP-18e, 2×25 mm).

General Method 13:

General Method 13 is similar to General Method 8, the coupling of an aminoboronate ester to a carboxylic acid followed by global acid deprotection, except that the global acid deprotection is carried out as follows. The protected amino acid is dissolved in EtOAc (approximately 0.1 M) and 4 M HCl (excess amount, generally 25 eq) in EtOAc was added dropwise at 0° C. After LCMS showed that the reaction was complete, the mixture was concentrated, and the residue was purified by preparative HPLC (CH₃CN/H₂O, with 0.1% HCl added to the mobile phase) to afford the desired product.

Compound 217 was prepared as the HCl salt using General Methods 1 and 13 from compound 217A and compound 126D2. LC-MS (General Method 12): MS (ESI) m/z 863.2 (M+H)⁺; t_{R} 0.762 min.

### Example 51

### Preparation of Compound 218

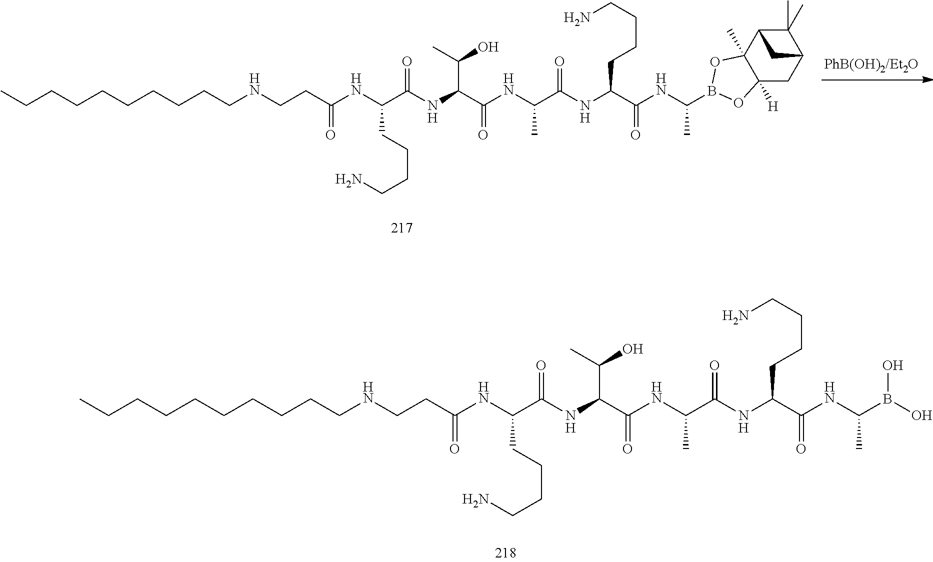

General Method 14:

To a solution of Compound 217 (500 mg, 0.58 mmol) in water (10 mL) was added phenyl boronic acid (212 mg, 3 eq) which was dissolved in water (5 mL) and ether (50 mL). The mixture was stirred at 25° C. overnight. After LC/MS analysis showed the reaction was completed, the water layer was evaporated under reduced pressure. The crude residue was washed with Et₂O, purified by prep-LCMS (0.1% HCl). LC-MS (General Method 12): MS (ESI) m/z 729.8 (M+H)⁺; t_{R} 0.707 min.

Alternatively, compound 218 can also be converted to the corresponding citric acid salt. To an aqueous solution of compound 218 (250 mg) was added citric acid (5 eq). The resulting mixture was then lyophilized to obtain a white solid.

### Example 52

### Preparation of Compound 219

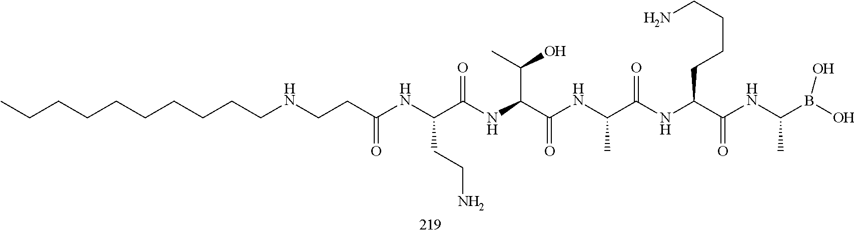 [SMILES: CCCCCCCCCCNCCC(=O)N[C@@H](CCN)C(=O)N[C@H](C(=O)N[C@@H](C)C(=O)N[C@@H](CCCCN)C(=O)N[C@@H](C)B(O)O)[C@@H](C)O]

Compound 219 was prepared as the HCl salt using General Methods 1, 13 and 14 from compound 217A. In this example, the free boronic acid was isolated during the preparative HPLC purification. LC-MS (General Method 12): MS (ESI) m/z 683.0 (M−H₂O+H)⁺; t_{R} 0.710 min.

### Example 53

### Preparation of Compound 220

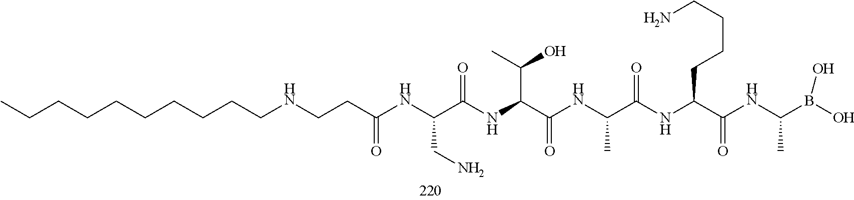 [SMILES: CCCCCCCCCCNCCC(=O)N[C@@H](CN)C(=O)N[C@H](C(=O)N[C@@H](C)C(=O)N[C@@H](CCCCN)C(=O)N[C@@H](C)B(O)O)[C@@H](C)O]

Compound 220 was prepared as the HCl salt using General Methods 1, 13 and 14 from compound 217A. In this example, the free boronic acid was isolated during the preparative HPLC purification. LC-MS (General Method 12): MS (ESI) m/z 669.4 (M−H₂O+H)⁺; t_{R} 0.707 min.

### Example 54

### Preparation of Compound 221

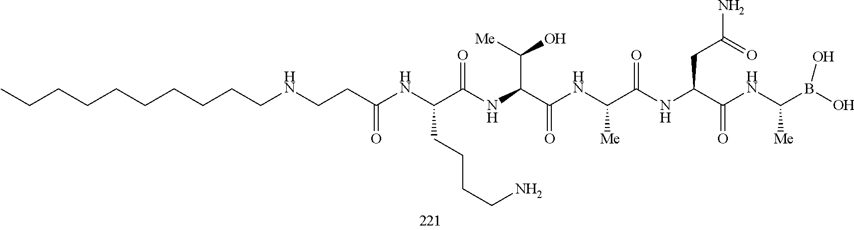 [SMILES: CCCCCCCCCCNCCC(=O)N[C@@H](CCCCN)C(=O)N[C@H](C(=O)N[C@@H](C)C(=O)N[C@@H](CC(N)=O)C(=O)N[C@@H](C)B(O)O)[C@@H](C)O]

Compound 221 was prepared as the HCl salt using General Methods 1, 13 and 14 from compound 217A. LC-MS (General Method 12): MS (ESI) m/z 697.1 (M−H₂O+H)⁺; t_{R} 0.716 min.

### Example 55

### Preparation of Compound 222

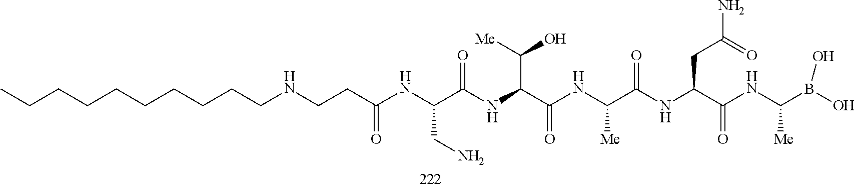 [SMILES: CCCCCCCCCCNCCC(=O)N[C@@H](CN)C(=O)N[C@H](C(=O)N[C@@H](C)C(=O)N[C@@H](CC(N)=O)C(=O)N[C@@H](C)B(O)O)[C@@H](C)O]

Compound 222 was prepared as the HCl salt using General Methods 1, 13 and 14 from compound 217A. LC-MS (General Method 12): MS (ESI) m/z 655.3 (M−H₂O+H)⁺; t_{R} 0.736 min.

### Example 56

### Preparation of Compound 223

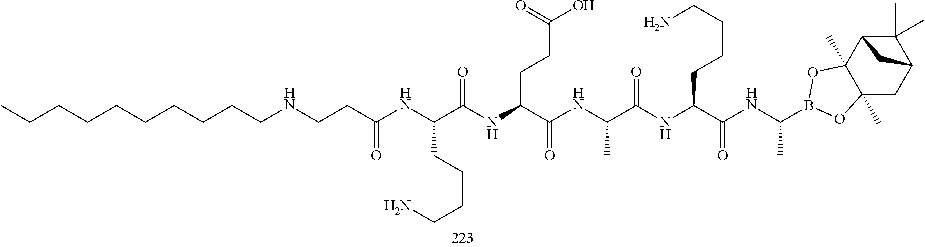 [SMILES: CCCCCCCCCCNCCC(=O)N[C@@H](CCCCN)C(=O)N[C@@H](CCC(=O)O)C(=O)N[C@@H](C)C(=O)N[C@@H](CCCCN)C(=O)N[C@@H](C)B1O[C@]2(C)C[C@@H]3C[C@@H](C3(C)C)[C@]2(C)O1]

Compound 223 was prepared as the HCl salt using General Methods 1 and 13 from compound 217A. LC-MS (General Method 12): MS (ESI) m/z 891.2 (M+H)⁺; t_{R} 0.763 min.

### Example 57

### Preparation of Compound 224

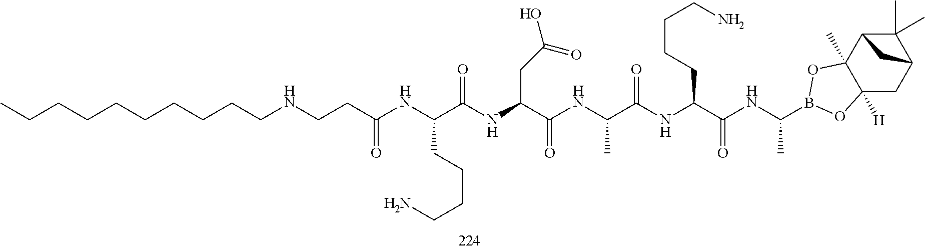 [SMILES: CCCCCCCCCCNCCC(=O)N[C@@H](CCCCN)C(=O)N[C@@H](CC(=O)O)C(=O)N[C@@H](C)C(=O)N[C@@H](CCCCN)C(=O)N[C@@H](C)B1O[C@@H]2C[C@@H]3C[C@@H](C3(C)C)[C@]2(C)O1]

Compound 224 was prepared as the HCl salt using General Methods 1 and 13 from compound 217A. LC-MS (General Method 12): MS (ESI) m/z 877.2 (M+H)⁺; t_{R} 0.771 min.

### Example 58

### Preparation of Compound 225

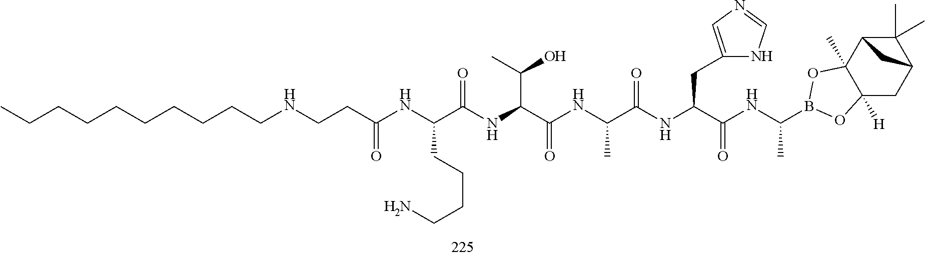 [SMILES: CCCCCCCCCCNCCC(=O)N[C@@H](CCCCN)C(=O)N[C@H](C(=O)N[C@@H](C)C(=O)N[C@@H](Cc1cnc[nH]1)C(=O)N[C@@H](C)B1O[C@@H]2C[C@@H]3C[C@@H](C3(C)C)[C@]2(C)O1)[C@@H](C)O]

Compound 225 was prepared as the HCl salt using General Methods 1 and 13 from compound 217A. LC-MS (General Method 12): MS (ESI) m/z 872.3 (M+H)⁺; t_{R} 0.764 min.

### Example 59

### Preparation of Compound 226

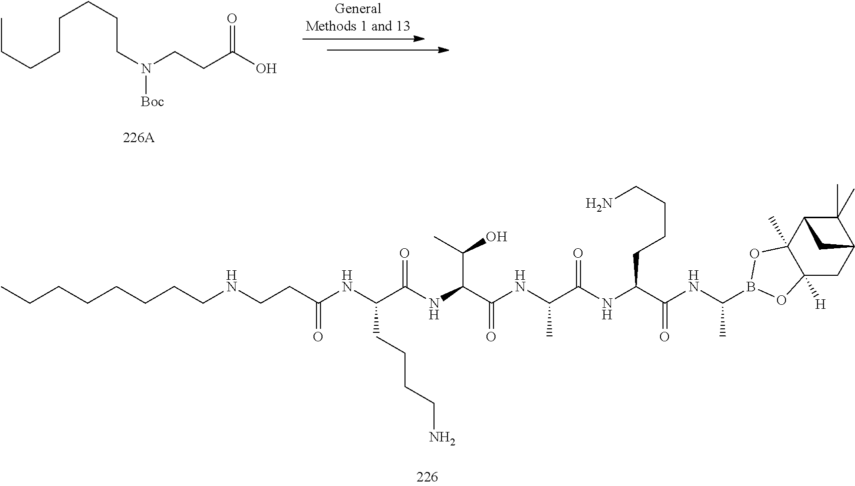 [SMILES: CCCCCCCCN(CCC(=O)O)C(=O)OC(C)(C)C ; CCCCCCCCNCCC(=O)N[C@@H](CCCCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 226A was prepared in a manner similar to compound 217A except octan-1-amine was used as the starting material. Compound 226 was prepared as the HCl salt using General Methods 1 and 13 from compound 226A and compound 126D2. LC-MS (General Method 12): MS (ESI) m/z 835.4 (M+H)⁺; t_{R} 0.737 min.

### Example 60

### Preparation of Compound 227 and 228

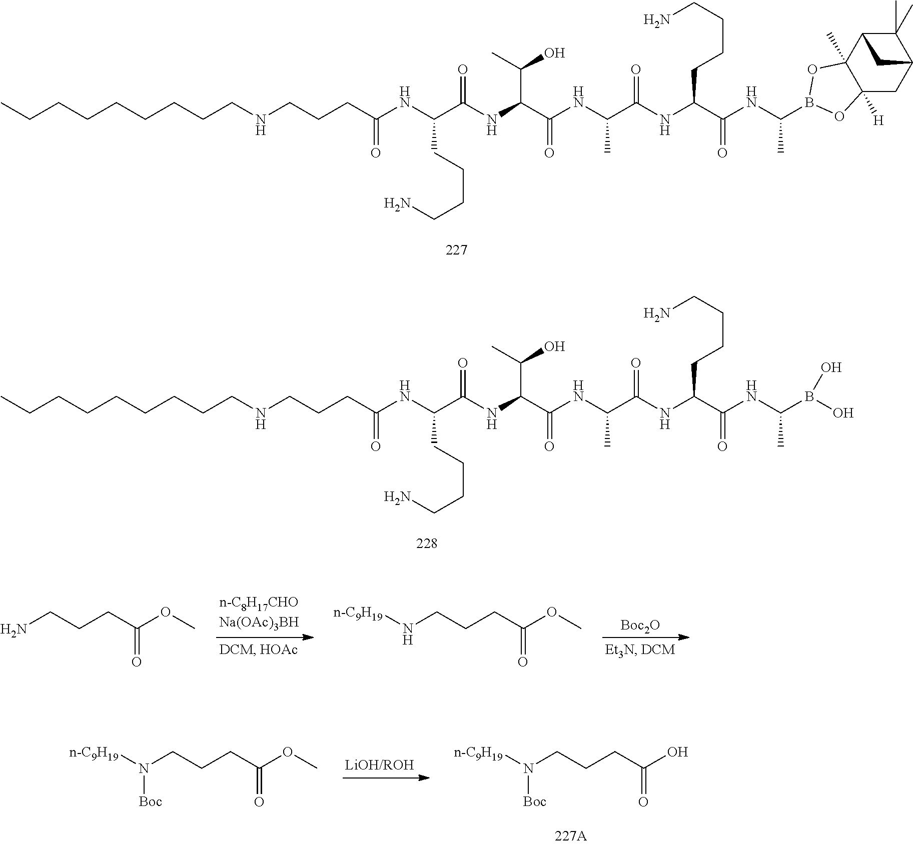

To a solution of nonanal (600 mg, 4.22 mmol) in DCM (25 mL) at 0° C. was added methyl 4-aminobutanoate (988 mg, 8.44 mmol) and HOAc (1 mL), followed by the addition of NaBH₃CN (398 mg, 2 mmol). The mixture was stirred at 15° C. for 12 h. After the reaction was complete, H₂O (20 mL) was added and the aqueous layer was extracted by DCM (30 mL*2). The combined organic layers were concentrated to obtain methyl 4-(nonylamino) butanoate.

The N-Boc formation and LiOH ester hydrolysis was performed in a manner similar to Compound 217A to afford 0.46 g of compound 227A. ELSD-LC/MS 352.3 (M+Na)⁺.

Compound 227 was prepared as the HCl salt using General Methods 1 and 13 from compound 227A and compound 126D2. LC-MS (General Method 12): MS (ESI) m/z 885.6 (M+H)⁺; t_{R} 0.755 min.

During the preparative HPLC purification of compound 227, compound 228 was also isolated as the free boronic acid. LC-MS (General Method 12): MS (ESI) m/z 711.3 (M+H)⁺; t_{R} 0.660 min.

### Example 61

### Preparation of Compound 229

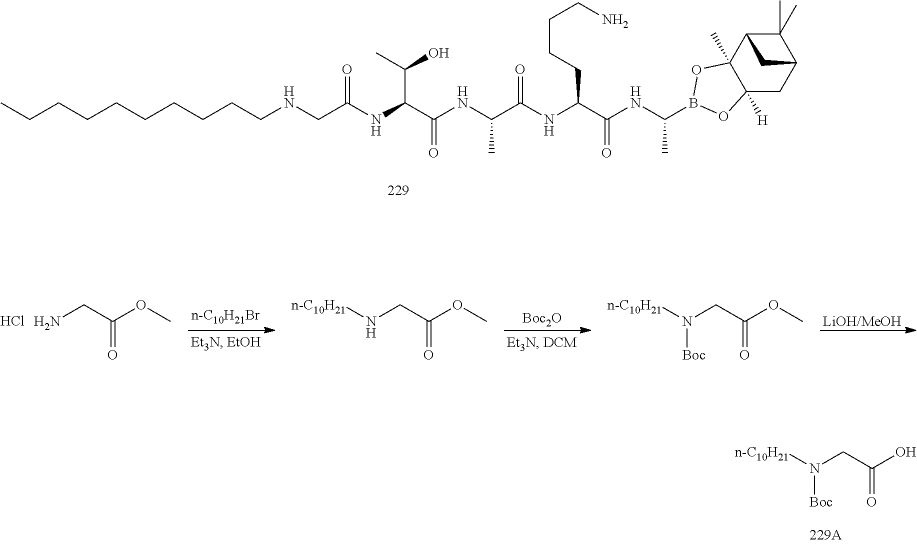

To a solution of decan-1-amine (10.5 g, 66.8 mmol) in anhydrous dichloromethane (250 mL) was added triethylamine (13.5 g, 133.5 mmol) at 0° C. and the reaction mixture was stirred at 0° C. for 30 min. Methyl bromoacetate (10.2 g, 66.8 mmol) was then added dropwise at 0° C., and the reaction mixture was stirred at room temperature for 14 h. The solution containing methyl 2-(decylamino)acetate was used directly for the next step.

The N-Boc formation and LiOH ester hydrolysis was performed in a manner similar to compound 217A, to afford 1.1 g of compound 229A. ¹H NMR (400 MHz, CDCl₃) δ 3.96 (s, 1H), 3.89 (s, 1H), 3.25-3.23 (m, 2H), 1.50-1.41 (m, 11H), 1.25 (m, 14H), 0.88-0.85 (t, J=6.8 Hz, 3H).

Compound 229 was prepared as the HCl salt using General Methods 1 and 13 from compound 229A. LC-MS (General Method 12): MS (ESI) m/z 721.4 (M+H)⁺; t_{R} 0.85 min.

### Example 62

### Preparation of Compound 230

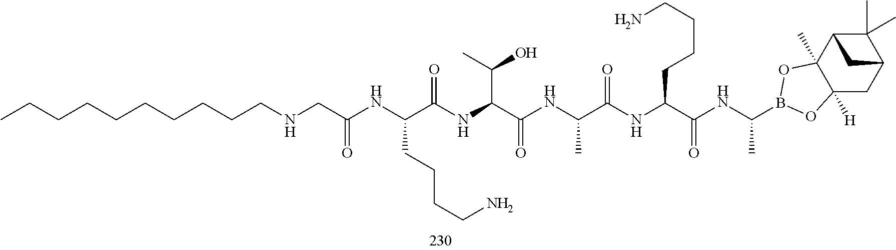 [SMILES: CCCCCCCCCCNCC(=O)N[C@@H](CCCCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 230 was prepared as the HCl salt using General Methods 1 and 13 from compound 229A and compound 126D2. LC-MS (General Method 12): MS (ESI) m/z 849.5 (M+H)⁺; t_{R} 0.748 min.

### Example 63

### Preparation of Compound 231

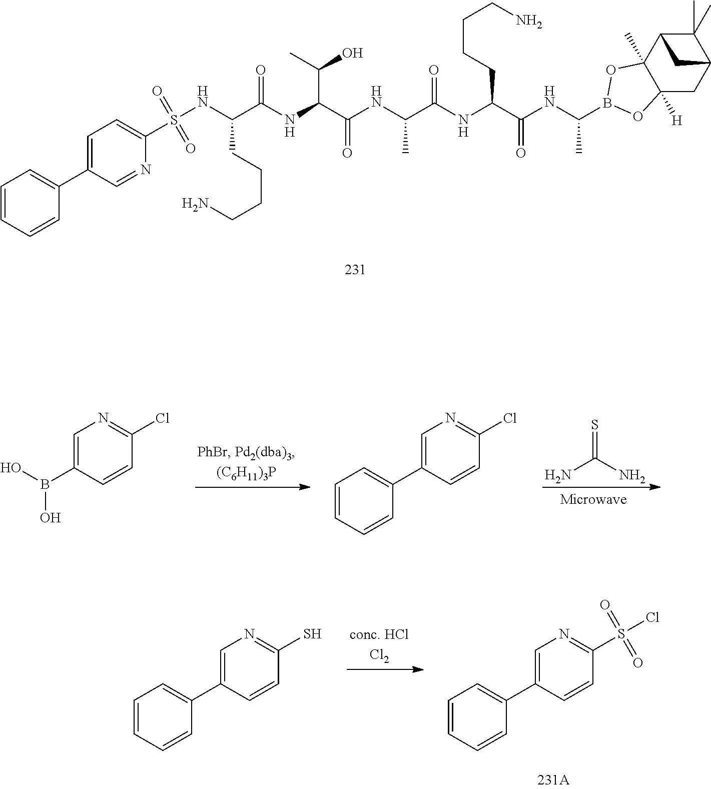

A mixture of (6-chloropyridin-3-yl)boronic acid (10 g, 63.6 mmol), phenyl bromide (10.5 g, 66.7 mmol), K₃PO₄ (22.9 g, 108 mmol), Pd₂(dba)₃ (2.3 g, 3.2 mmol), (C₆H₁₁)₃P (1.8 g, 6.4 mmol) in 1,4-dioxane/H₂O (220 mL/22 mL) under N₂ protection was stirred at 100° C. for 15 h. After TLC showed that the starting materials were consumed completely, the mixture was filtered through a celite pad and the filtrate was concentrated under reduced pressure. The residue was then purified by silica gel column chromatography (PE:EA=19:1 to 9:1 to 3:1) to give 2-chloro-5-phenylpyridine (10 g, yield: 83%) as a yellow solid.

To the prepared sealed vial was added a solution of 2-chloro-5-phenylpyridine (1 g, 5.3 mmol) and thiourea (0.8 g, 10 mmol) in NMP (15 mL). The mixture was irradiated in the microwave on a Biotage Smith Synthesizer at 195° C. for 15 min. After TLC showed that the starting materials were consumed completely, the reaction solution was cooled down and quenched with water (100 mL), which was then extracted with ethyl acetate (30 mL×3). The combined organic layers were washed with brine (60 mL), dried over Na₂SO₄, and then concentrated under reduced pressure. The residue was purified by silica gel column chromatography (PE:EA=10:1 to 5:1 to 1:1) to give 5-phenylpyridine-2-thiol (285 mg, yield: 25%) as a yellow solid.

A stream of Cl₂ was bubbled through a cold (−15° C.) solution of 5-phenylpyridine-2-thiol (1.7 g, 5.3 mmol) in conc. HCl (25 mL) for 30 min. Then the solution was purged with nitrogen for 1 min to remove excess of Cl₂. The resulting precipitate was then filtered, washed by water (20 mL×2) and dried to obtain compound 231A (2 g, 88%) as a yellow solid.

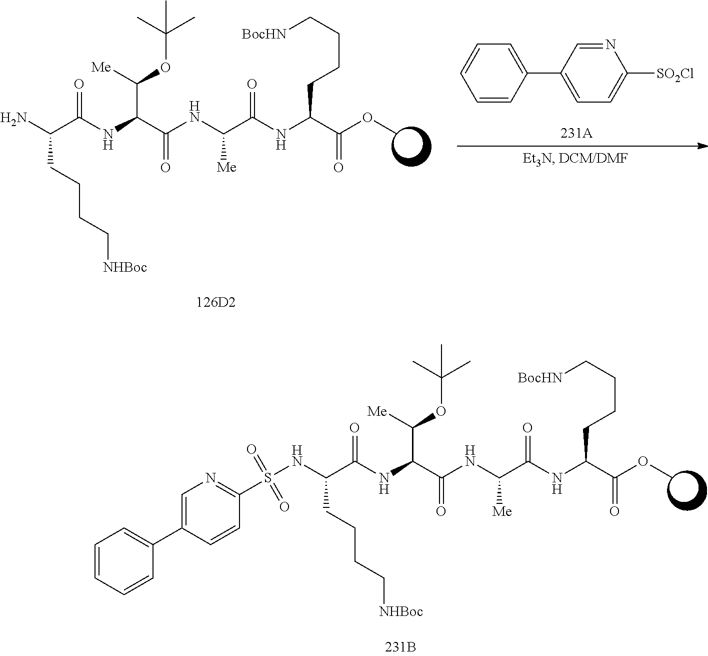 [SMILES: COC(=O)[C@H](CCCCNC(=O)OC(C)(C)C)NC(=O)[C@H](C)NC(=O)[C@@H](NC(=O)[C@@H](N)CCCCNC(=O)OC(C)(C)C)[C@@H](C)OC(C)(C)C ; O=S(=O)(Cl)c1ccc(-c2ccccc2)cn1 ; COC(=O)[C@H](CCCCNC(=O)OC(C)(C)C)NC(=O)[C@H](C)NC(=O)[C@@H](NC(=O)[C@H](CCCCNC(=O)OC(C)(C)C)NS(=O)(=O)c1ccc(-c2ccccc2)cn1)[C@@H](C)OC(C)(C)C]

To a solution of compound 126D2 (3.2 g, 3.6 mmol) and triethylamine (1.5 g, 14.4 mmol) in DMF/DCM (15 ml/5 ml) was added compound 231A (1.8 g, 7.2 mmol) at 0° C. The mixture was stirred at 30° C. for 15 h. The mixture was filtered and the solid was washed with DCM (30 ml)/MeOH (30 mL) to give compound 231B (3.4 g, 68%). Compound 231 was prepared as the HCl salt using General Methods 1 and 13 from compound 231B. LC-MS (General Method 12): MS (ESI) m/z 869.7 (M+H)⁺; t_{R} 0.674 min.

### Example 64

### Preparation of Compound 232

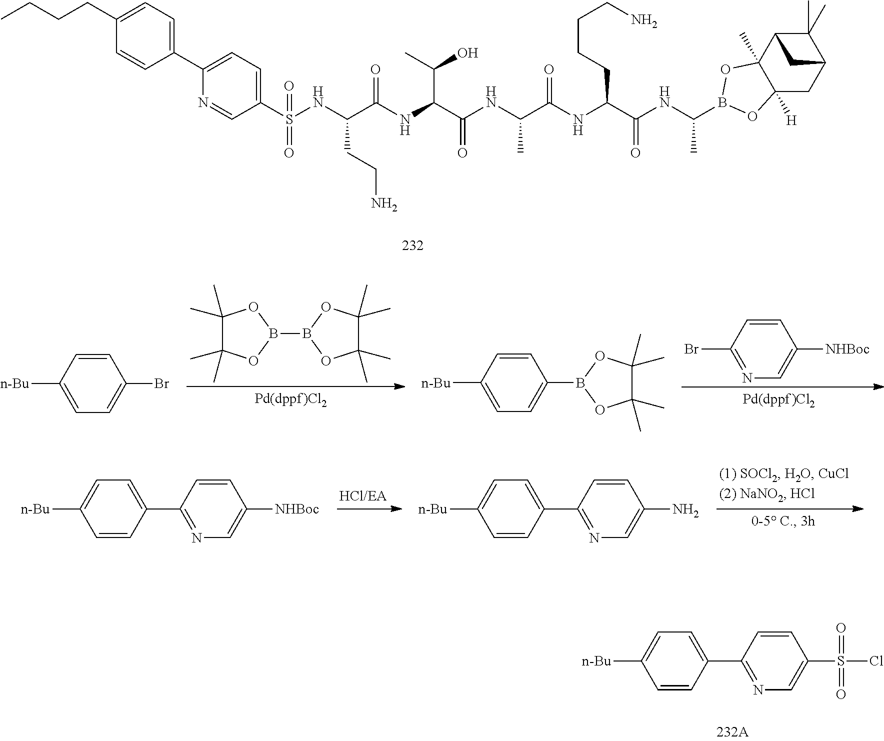

To a stirred of 1-bromo-4-butylbenzene (5.8 g, 27.2 mmol), bis(pinacolato)diboron (8.3 g, 32.6 mmol), Cs₂CO₃ (17.7 g, 54.4 mmol) in 1,4-dioxane/H₂O (200 mL/20 mL) under N₂, Pd(dppf)Cl₂ (2.0 g, 2.72 mmol) was added. The mixture was stirred at 90° C. for 16 h under N₂. The mixture was added to 300 mL water, which was then extracted with ethyl acetate (300 mL×3). The combined organic layers were concentrated. The residue was purified by column (PE: EA=50:1) to give 2-(4-butylphenyl)-4,4,5,5-tetramethyl-1,3,2-dioxaborolane (5.8 g, 75%) as clear oil residue.

To a stirred of 2-(4-butylphenyl)-4,4,5,5-tetramethyl-1,3,2-dioxaborolane (5.8 g, 22.3 mmol), tert-butyl (6-bromopyridin-3-yl)carbamate (6.1 g, 22.3 mmol), Cs₂CO₃ (14.5 g, 44.6 mmol) in 1,4-dioxane/H₂O (200 mL/20 mL) under N₂, Pd(dppf)Cl₂ (1.6 g, 2.23 mmol) was added. The mixture was stirred at 90° C. for 16 hours under N₂. The mixture was added to 300 ml water, which was then extracted with ethyl acetate (300 mL×3). The combined organic layers were concentrated. The residue was purified by column (PE: EA=5:1) to give tert-butyl (6-(4-butylphenyl)pyridin-3-yl)carbamate (3.6 g, 49.5%) as pale yellow solid.

A solution of tert-butyl (6-(4-butylphenyl)pyridin-3-yl)carbamate (3.6 g, 11 mmol) in 4 N HCl/EtOAc (100 mL) was stirred in an ice-bath for 30 minutes and then stirred at 30° C. for 16 hours. The mixture was concentrated to give 6-(4-butylphenyl)pyridin-3-amine (3.0 g, 91%) as pale yellow solid.

A solution of SO₂ was prepared by adding SOCl₂ (5 mL) into stirring water (30 mL) containing CuCl (0.032 g, 0.32 mmol). The solution was then stirred at 30° C. for 18 h. 6-(4-butylphenyl)pyridin-3-amine (3.0 g, 13.3 mmol) was added into stirring conc. HCl (16.5 mL) portion wise and the resulting mixture was added dropwise to a solution of NaNO₂ (1.20 g, 17.5 mmol) in 5 mL water while maintaining temperature 0-5° C. The resulting mixture was stirred for 30 min after the completion of the addition and then added dropwise into the aqueous solution of 5O₂. The temperature was kept below 0° C. during the addition. After that, the mixture was stirred for 1 h below 0° C. and then filtered. The cake was washed with ice-cold water, dissolved in CH₂Cl₂ (50 mL), dried over Na₂SO₄ and concentrated to give crude compound 232A as pale yellow oil (2.3 g, 56%).

Compound 232 was prepared as the HCl salt using General Methods 1 and 13 from compound 232A and compound 126D2 similar to that described for Compound 231. LC-MS (General Method 12): MS (ESI) m/z 896.5 (M+H)⁺; t_{R} 0.816 min.

### Example 65

### Preparation of Compound 233

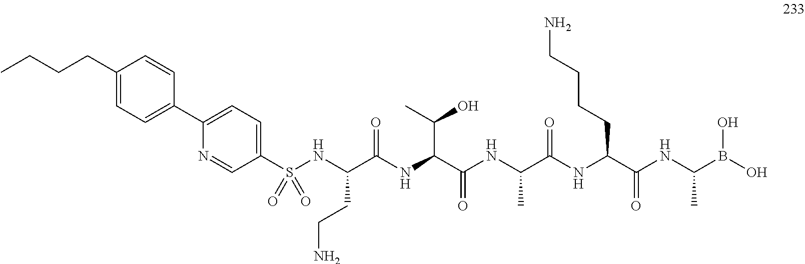 [SMILES: CCCCc1ccc(-c2ccc(S(=O)(=O)N[C@@H](CCN)C(=O)N[C@H](C(=O)N[C@@H](C)C(=O)N[C@@H](CCCCN)C(=O)N[C@@H](C)B(O)O)[C@@H](C)O)cn2)cc1]

Compound 233 was prepared from Compound 232 according to General Method 14. LC-MS (General Method 12): MS (ESI) m/z 744.9 (M−H₂O+H)⁺; t_{R} 0.740 min.

### Example 66

### Preparation of Compound 234 and Compound 235

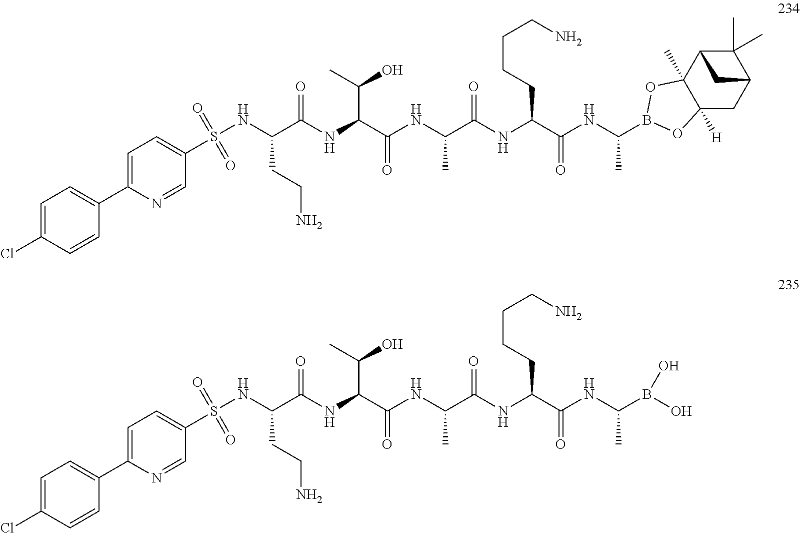 [SMILES: C[C@H](NC(=O)[C@H](CCCCN)NC(=O)[C@H](C)NC(=O)[C@@H](NC(=O)[C@H](CCN)NS(=O)(=O)c1ccc(-c2ccc(Cl)cc2)nc1)[C@@H](C)O)B1O[C@@H]2C[C@@H]3C[C@@H](C3(C)C)[C@]2(C)O1 ; C[C@H](NC(=O)[C@H](CCCCN)NC(=O)[C@H](C)NC(=O)[C@@H](NC(=O)[C@H](CCN)NS(=O)(=O)c1ccc(-c2ccc(Cl)cc2)nc1)[C@@H](C)O)B(O)O]

Compound 234 was prepared as the HCl salt using General Methods 1 and 13 in a manner similar to compound 233 except 1-bromo-4-chlorobenzene was used as the starting material. LC-MS (General Method 12): MS (ESI) m/z 874.9 (M+H)⁺; t_{R} 0.790 min.

During the preparative HPLC purification of compound 234, compound 235 was also isolated as the free boronic acid. LC-MS (General Method 12): MS (ESI) m/z 722.9 (M−H₂O+H)⁺; t_{R} 0.699 min.

### Example 67

### Preparation of Compound 236

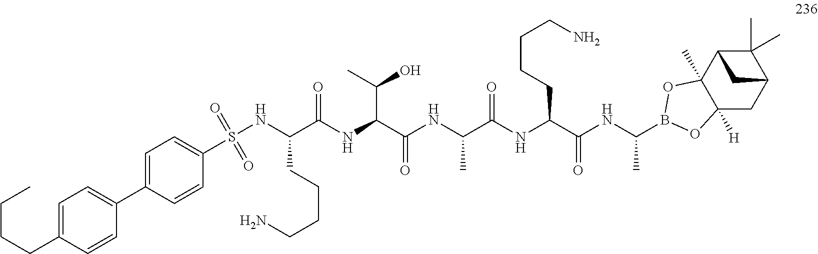 [SMILES: CCCCc1ccc(-c2ccc(S(=O)(=O)N[C@@H](CCCCN)C(=O)N[C@H](C(=O)N[C@@H](C)C(=O)N[C@@H](CCCCN)C(=O)N[C@@H](C)B3O[C@@H]4C[C@@H]5C[C@@H](C5(C)C)[C@]4(C)O3)[C@@H](C)O)cc2)cc1]

Compound 236 was prepared as the HCl salt using General Methods 1 and 13 in a manner similar to compound 128 except 1-bromo-4-butylbenzene was used as the starting material. LC-MS (General Method 12): MS (ESI) m/z 924.3 (M+H)⁺; t_{R} 0.827 min.

### Example 68

### Preparation of Compound 237

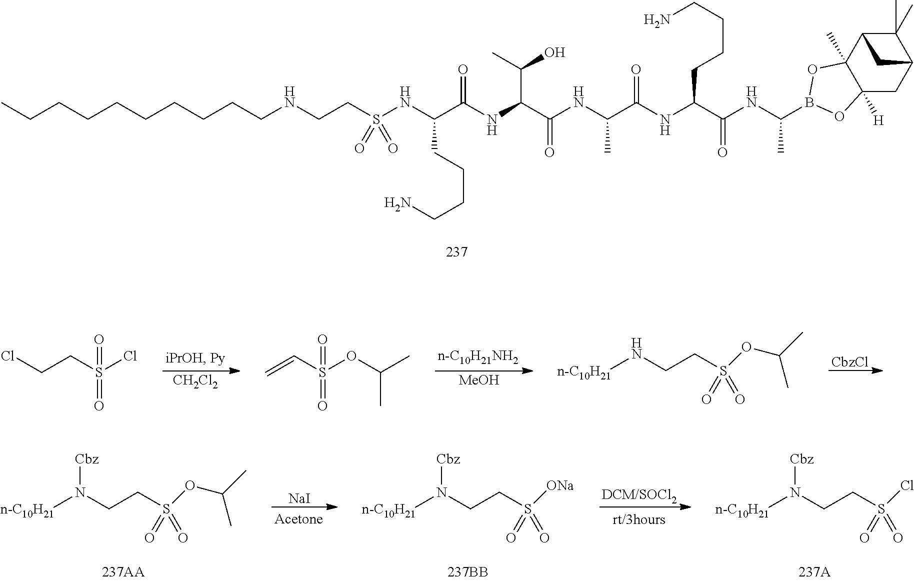

To a solution of 2-chloroethanesulfonyl chloride (25.0 g, 153 mmol) and pyridine (24.3 g, 307 mmol) in DCM (200 mL) was added i-PrOH (27.6 g, 460 mmol) at 0° C. The mixture was warmed to room temperature and stirred for another 3 h at the same temperature. The reaction was washed sequentially with 1N HCl (200 mL) and saturated NaHCO₃ (100 mL). The aqueous layer was further extracted by DCM (200 mL*2). The combined DCM layers were concentrated and the residue was purified by silica gel column (PE/EA=5/1) to afford isopropyl ethenesulfonate (19.5 g, 84.7%) as a colorless oil. ¹H NMR (400 MHz, CDCl₃) δ 0.81 (d, J=4.2 Hz, 6H), 4.74-4.83 (m, 1H), 6.05 (1H, d, J=10.0 Hz), 6.38 (1H, d, J=12.4 Hz), 6.54 (1H, dd, J=12.4 Hz, J=10.0 Hz).

To a solution of n-C₁₀H₂₁NH₂ (19.0 g, 121 mmol) in MeOH (200 mL) at 0° C. was added isopropyl ethenesulfonate (18.2 g, 121 mmol) dropwise. The mixture was warmed to room temperature slowly and stirred for two days at room temperature. The reaction mixture was then concentrated and the residue was purified by silica gel chromatography (PE/EA=20/1 to 3/1) to afford isopropyl 2-(decylamino)ethanesulfonate (20.2 g, 54.4%) as a colorless oil. ¹H NMR (400 MHz, CDCl₃) δ 0.81 (3H, t, J=6.8 Hz), 1.36-1.40 (m, 2H), 2.54 (2H, t, J=7.2 Hz), 3.03 (2H, t, J=7.2 Hz), 3.20 (2H, t, J=4.5 Hz), 4.87-4.95 (m, 1H).

To a solution of isopropyl 2-(decylamino)ethanesulfonate (20.0 g, 65 mmol) and Et₃N (13.2 g, 130 mmol) in DCM (200 mL) at 0° C. was added CbzCl (12.2 g, 71.6 mmol) dropwise. The mixture was warmed to room temperature slowly and stirred for two days at room temperature. The reaction was washed sequentially with 1N HCl (200 mL) and saturated NaHCO₃ (100 mL). The aqueous layer was further extracted by DCM (200 mL*2). The combined DCM layers were concentrated and the residue was purified by silica gel column (PE/EA=10/1) to afford Compound 237AA (28.0 g, 96.6%) as a colorless oil. ¹H NMR (400 MHz, CDCl₃) δ 0.81 (3H, t, J=6.8 Hz), 1.17-1.20 (m, 16H), 1.14-1.15 (m, 2H), 3.15-3.33 (m, 4H), 3.59-3.62 (m, 2H), 4.85-4.89 (m, 1H), 5.06 (s, 2H), 7.21-7.30 (m, 5H).

To a solution of compound 237AA (28.0 g, 63.4 mmol) in acetone (300 mL) was added NaI (11.4 g, 76.1 mmol) in one portion. The reaction mixture was heated to reflux overnight. The reaction mixture was cooled to room temperature and the resulting solid was filtered and dried in vacuum to give compound 237BB (26.1 g, 97.8%) as a white solid. ¹H NMR (400 MHz, DMSO) δ 0.82 (3H, t, J=6.8 Hz), 1.20 (br, 14H), 1.39-1.43 (m, 2H), 2.58-2.64 (m, 2H), 3.14-3.18 (m, 2H), 5.03 (s, 2H), 7.27-7.35 (m, 5H).

To a mixture of compound 237BB (7.0 g, 25.0 mmol) in DCM (100 mL) and DMF (0.1 mL) at 0° C. was added SOCl₂ (29.7 g, 250 mmol). The mixture was slowly warmed to room temperature and stirred for 3 h at room temperature. The reaction mixture was concentrated in vacuum to afford compound 237A, which was used directly without further purification.

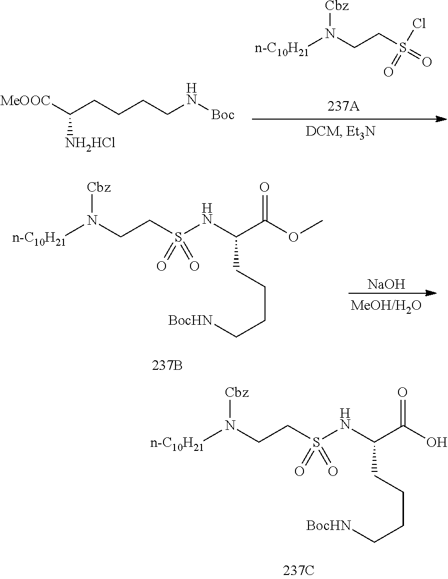

To a mixture of compound L-Lys(Boc)-OMe HCl (2.0 g, 7.68 mmol) and Et₃N (2.33 g 23.05 mmol) in DCM (40 mL) was added a solution of compound 237A (6.42 g, 15.37 mmol) in DCM (20 mL) dropwise at 0° C. The mixture was warmed to room temperature slowly and stirred overnight at room temperature. The reaction was diluted with DCM (200 mL) and washed with 2N HCl (30 mL×3), NaHCO₃ (30 mL). The DCM layer was separated, dried with Na₂SO₄, and concentrated under reduced pressure. The residue was further purified by silica gel chromatography (PE:EA=5:1) to obtained compound 237B (1.7 g, 44.2%) as colorless oil. ¹H NMR (400 MHz, DMSO) δ 7.28 (br, s, 5H), 5.07 (s, 2H), 3.84 (s, 3H), 3.57-3.62 (m, 3H), 3.36-3.57 (m, 1H), 3.20-3.24 (m, 3H), 1.44-1.46 (m, 4H), 1.18-1.26 (m, 28H), 0.82 (3H, t, J=6.8 Hz).

A solution of compound 237B (1.7 g, 8.87 mmol) in MeOH (5 mL) and H₂O (5 mL) was treated with NaOH (1.36 g, 88.7 mmol) at 0° C. The mixture was warmed to room temperature slowly and stirred overnight at room temperature. The reaction mixture was concentrated in vacuum to remove most of the MeOH; the residue was adjust the pH to 1 with 2N HCl, and filtered to obtain compound 237C (0.9 g, 54.5%) as a white solid. The compound was used directly without further purification.

Compound 237 was prepared as the HCl salt using General Methods 1 and 13 from compound 237C and compound 126D2. LC-MS (General Method 12): MS (ESI) m/z 899.7 (M+H)⁺; t_{R} 0.776 min.

### Example 69

### Preparation of Compound 238 (HCl Salt)

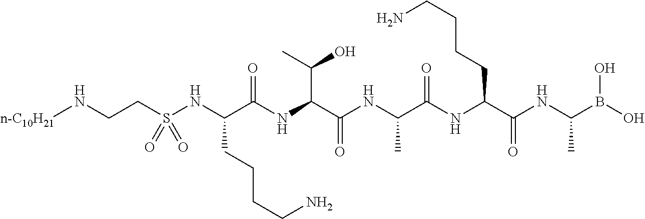

Compound 238 was isolated as a by-product during the prep HPLC purification of Compound 237. LC-MS (General Method 12): MS (ESI) m/z 747.5 (M+H)⁺; t_{R} 0.707 min.

### Example 70

### Preparation of Compound 239

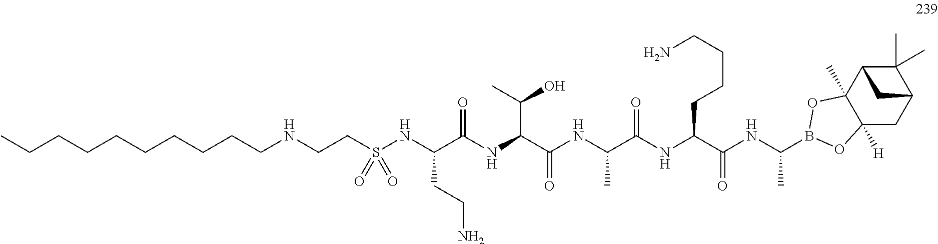 [SMILES: CCCCCCCCCCNCCS(=O)(=O)N[C@@H](CCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 239 was prepared as the HCl salt using General Methods 1 and 13 from compound 237C in a manner similar to compound 237. LC-MS (General Method 12): MS (ESI) m/z 871.4 (M+H)⁺; t_{R} 0.768 min.

### Example 71

### Preparation of Compound 240

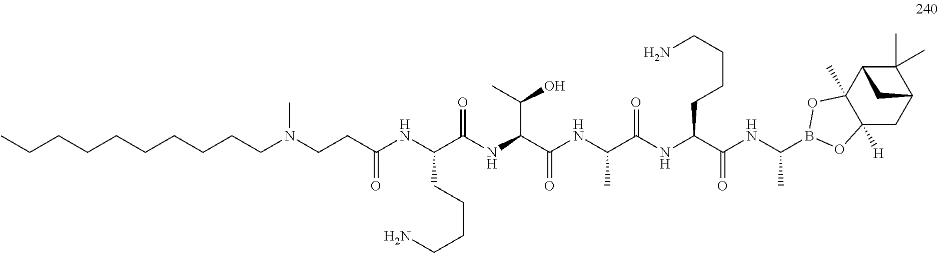 [SMILES: CCCCCCCCCCN(C)CCC(=O)N[C@@H](CCCCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

1-bromodecane (22 g, 100 mmol) was added to a solution of methylamine (30˜34% solution in ethanol, 200 mL) and the mixture was stirred at room temperature under N₂ for 18 h. After TLC showed the reaction was completed, the reaction mixture was evaporated to remove the solvent under vacuum to give N-methyldecan-1-amine as yellow oil without further purification.

To a solution of methyl acrylate (8.6 g, 100 mmol) in THF (100 mL) cooled at 0° C. was added a solution of N-methyldecan-1-amine (17 g, 100 mmol) in THF (50 mL). The reaction mixture was stirred at room temperature for 18 h. After TLC showed the reaction was completed, the reaction mixture was evaporated to remove the solvent under vacuum and the residue was purified on silica gel column to obtain methyl 3-(decyl(methyl)amino)propanoate as colorless oil (4.5 g, 17.5%).

To a solution of methyl 3-(decyl(methyl)amino)propanoate (1.5 g, 6 mmol) in EtOH (15 mL) and H₂O (10 mL) was added LiOH (0.43 g, 18 mmol). The mixture was stirred at room temperature for 16 h. After that, the reaction mixture was concentrated to remove EtOH and the residue was added H₂O (50 mL), which was adjusted to pH=1˜2 with 6N HCl. The aqueous layer was then extracted with EtOAc (50 mL×3). The combined organic layers were dried and concentrated to give compound 240A as colorless oil (0.8 g, 54.8%).

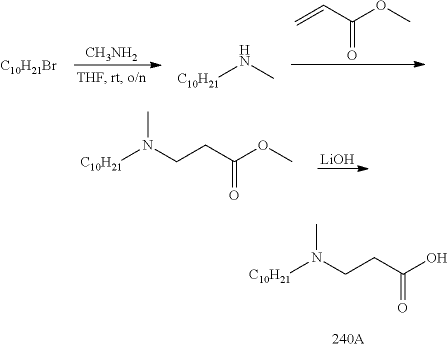

Compound 240 was prepared as the HCl salt using General Methods 1 and 13 from compound 240A and compound 126D2. LC-MS (General Method 12): MS (ESI) m/z 877.5 (M+H)⁺; t_{R} 0.769 min.

### Example 72

### Preparation of Compound 241

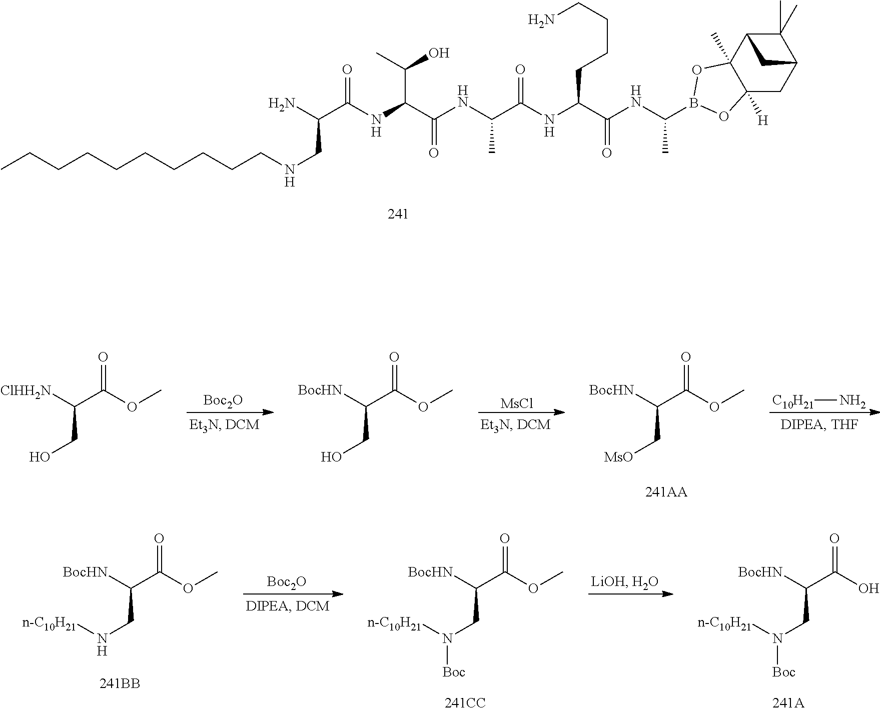

To a solution of D-Ser-OMe HCl (20 g, 129 mmol) and Et₃N (32.5 g, 322 mmol) in DCM (200 mL) was added Boc₂O (33.7 g, 154 mmol) dropwise at ice-bath, and then the mixture was stirred at 25° C. for 16 h. The mixture were concentrated and the residue was further purified by silica gel column (PE:EA=10:1 to 5:1) to give Boc-D-Ser-OMe as colorless oil (19.5 g, 69%).

To a solution of Boc-D-Ser-OMe (10 g, 45.6 mmol) and Et₃N (5.5 g, 54.7 mmol) in DCM (100 mL) was added a solution of MsCl (6.2 g, 54.7 mmol) in DCM (10 mL) dropwise at 0° C. After stirring at 25° C. for 16 h, the reaction mixture was concentrated to remove the solvent. And the residue was re-dissolved in DCM (200 mL) and washed with brine (200 mL×2), dried over Na₂SO₄, and concentrated. The residue was purified by silica gel column (PE: EtOAc=6:1 to 3:1) to afford compound 241AA as a colorless oil (9.5 g, 70%).

A solution of compound 241AA (3.1 g, 10.4 mmol) and decan-1-amine (2.46 g, 15.6 mmol) in THF (50 mL) was heated to 80° C. for 16 hours. After TLC showed the reaction was completed, the mixture was concentrated and the residue was purified by silica gel column (PE:EA=5:1 to 2:1) to give compound 241BB as colorless oil (1.5 g, 40%).

To a solution of compound 241BB (1.8 g, 5.0 mmol) and Et₃N (0.76 g, 7.5 mmol) in DCM (50 mL) was added Boc₂O (1.6 g, 7.5 mmol) dropwise at ice-bath, and then the mixture was stirred at 25° C. for 16 hours. After TLC showed the reaction was completed, the mixture was concentrated and the residue was purified by silica gel column (PE:EA=5:1) to give compound 240CC as colorless oil (2.1 g, 92%).

To a stirred solution of compound 241CC (2.2 g, 4.65 mmol) in EtOH/H₂O (40 mL/20 mL), LiOH.H₂O (0.37 g, 9.31 mmol) was added. The mixture was stirred at 25° C. for 16 hours. The reaction mixture was concentrated under reduced pressure to remove EtOH and then was added H₂O (50 mL), which was extracted by DCM (50 mL). The aqueous was then adjusted to pH=1˜2 with 6N HCl, which was further extracted with EtOAc (50 mL×3). The combined organic layers were concentrated to give compound 241A as colorless oil (2.0 g, 96%).

Compound 241 was prepared as the HCl salt using General Methods 1 and 13 from compound 241A and compound 101C2. LC-MS (General Method 12): MS (ESI) m/z 750.2 (M+H)⁺; t_{R} 0.782 min.

### Example 73

### Preparation of Compound 242

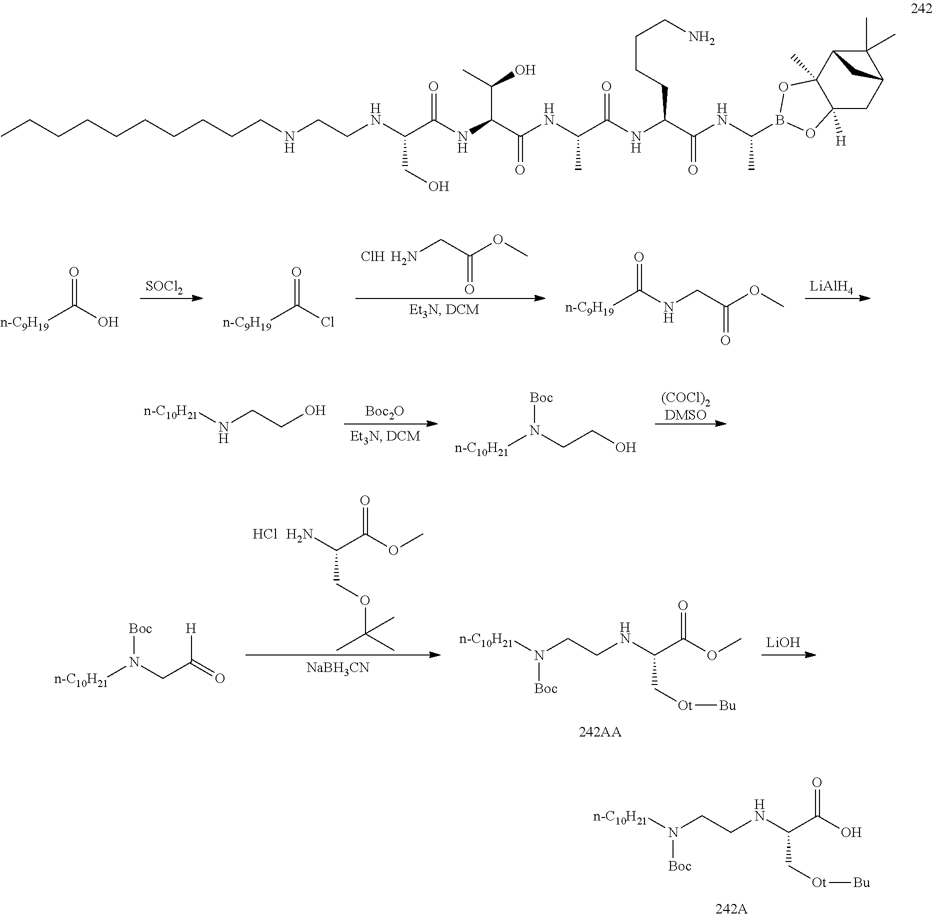

A solution of decanoic acid (30 g, 174 mmol) in SOCl₂ (100 mL) was refluxed for 3 h. After removing excess SOCl₂ under reduced pressure, the residue was re-dissolved in dry DCM (50 mL) and then added dropwise into a solution of glycine methyl ester HCl (21.7 g, 174 mmol) and Et₃N (52.7 g, 522 mmol) in DCM (200 mL) at 0° C. The mixture was then allowed to warm to 25° C. and stirred for 16 h at the same temperature. The reaction mixture was washed with brine (200 mL) and the organic layer was dried over Na₂SO₄, concentrated under reduced pressure. The residue was purified by silica gel column (PE:EA=10:1 to 5:1) to give methyl 2-decanamidoacetate as a white solid (34 g, 80%).

THF (200 ml) was slowly added to an argon purged flask containing LiAlH₄ (15.9 g, 420 mmol). This suspension was brought to reflux and a solution of methyl 2-decanamidoacetate (34 g, 140 mmol) in THF (100 mL) was added via addition funnel over 30 min and stirring at reflux for 16 h. After cooling down, a solution of water (32 ml) in THF (50 ml) was added dropwise to the reaction mixture while maintaining an internal temperature below 20° C. Additional THF (200 mL) was added in portions to maintain consistent stirring, followed by the addition of 10% NaOH (16 mL) dropwise. The reaction mixture was brought to reflux for 1 h at which point the solid in suspension turned completely white. The mixture was filtered through a Buchner funnel and the filtrate was concentrated under reduced pressure. The resulting residue is taken up in 200 mL EtOAc, dried over MgSO₄, and concentrated to obtain 2-(decylamino)ethanol as a colorless oil (22 g, 78.6%).

A solution of 2-(decylamino)ethanol (22 g, 109 mmol) and Et₃N (11 g, 109 mmol) in DCM (200 mL) was added Boc₂O (23.8 g, 109 mmol) dropwise at 0° C., and then the mixture was stirred at 20° C. for 16 hours. The mixture was concentrated and the residue was purified by silica gel column (PE:EA=5:1) to give tert-butyl decyl(2-hydroxyethyl)carbamate as a clear oil (29.5 g, 90%).

To a stirring solution of oxalyl chloride (22.7 g, 179 mmol) in DCM (200 mL) at −50° C. was added a solution of DMSO (18.7 g, 239 mmol) in DCM (50 mL) via addition funnel. After stirring for 15 min, a solution of tert-butyl decyl(2-hydroxyethyl)carbamate (18 g, 59.7 mmol) in DCM (50 mL) was added to the reaction over 10 min and the mixture was held at −50° C. to −45° C. for 2 h. After that, the reaction mixture was diluted with DCM (50 mL) and triethylamine (27.1 g, 269 mmol) was added slowly via addition funnel. And the mixture was maintained at −25° C. for 30 min. The reaction was quenched by the addition of 1M NaHSO₄, and the mixture was stirred for 30 min. The organic layer was further washed with 1M NaHSO₄, saturated NaHCO₃, and brine, dried over MgSO₄, and concentrated under reduced pressure to provide 2-(decylamino)acetaldehyde as a colorless oil (16.5 g, 92.3%).

A solution of 2-(decylamino)acetaldehyde (3.0 g, 10.0 mmol), L-Ser(tBu)-OMe.HCl (2.11 g, 10.0 mmol) and DIPEA (1.29 g, 10 mmol) in DCM/MeOH (90 mL/30 mL), and the mixture was stirred at 15° C. for 30 min. NaBH₃CN (1.89 g, 30 mmol) and AcOH (0.5 mL) were added to the mixture. And the reaction mixture was stirred at 15° C. for 1.5 h. The mixture was then concentrated and the residue was added 100 ml water, which was extracted with ethyl acetate (100 mL×3). The combined organic layers were dried and concentrated. The residue was purified by silica gel column (PE:EA=10:1 to 6:1) to give compound 242AA as colorless oil (2.5 g, 55%).

To a stirred solution of compound 242AA (2.5 g, 5.45 mmol) in EtOH/H₂O (40 mL/20 mL), LiOH.H₂O (458 mg, 10.9 mmol) was added. The mixture was stirred at room temperature for 16 h. After that, the reaction mixture was concentrated to remove EtOH, then H₂O (100 mL) was added. The pH was adjusted to 1˜2 by addition of 6N HCl. The aqueous layer was extracted with EtOAc (100 mL×3). The combined organic layers were dried and concentrated to give compound 242A as colorless oil (1.9 g, 79%).

Compound 242 was prepared as the HCl salt using General Methods 1 and 13 from compound 242A and compound 101C2. LC-MS (General Method 12): MS (ESI) m/z 794.1 (M+H)⁺; t_{R} 0.784 min.

### Example 74

### Preparation of Compound 243

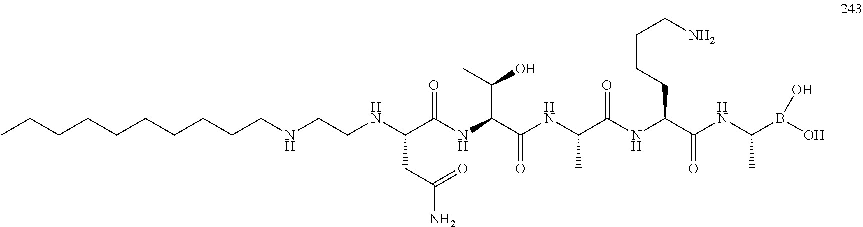 [SMILES: CCCCCCCCCCNCCN[C@@H](CC(N)=O)C(=O)N[C@H](C(=O)N[C@@H](C)C(=O)N[C@@H](CCCCN)C(=O)N[C@@H](C)B(O)O)[C@@H](C)O]

Compound 243 was prepared as the HCl salt using General Methods 1, 13 and 14 in a manner similar to compound 242 except the synthesis starting from L-Asn(Trt)-OMe. LC-MS (General Method 12): MS (ESI) m/z 687.4 (M+H)⁺; t_{R} 0.728 min.

### Example 75

### Preparation of Compound 244

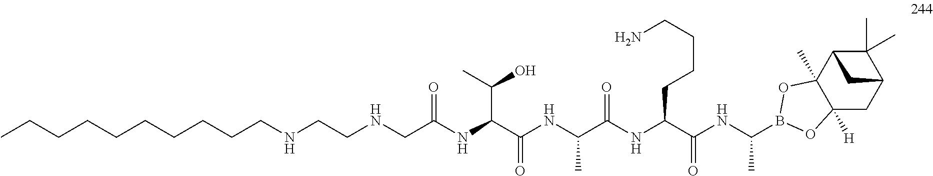 [SMILES: CCCCCCCCCCNCCNCC(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 244 was prepared as the HCl salt using General Methods 1 and 13 in a manner similar to compound 242 except the synthesis begins with Gly-(OMe). LC-MS (General Method 12): MS (ESI) m/z 764.2 (M+H)⁺; t_{R} 0.765 min.

### Example 76

### Preparation of Compound 245

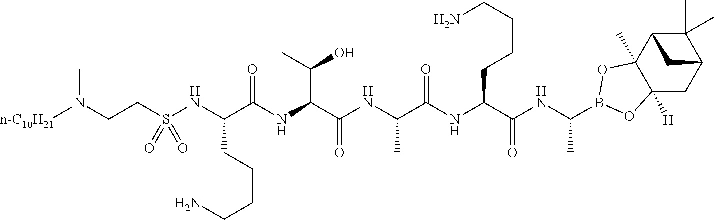

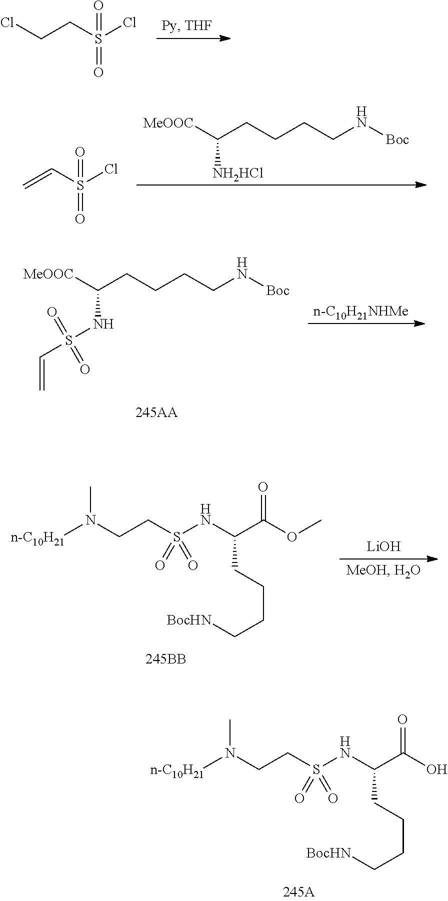

To a solution of 2-chloroethanesulfonyl chloride (2 g, 12.3 mmol) in dichloromethane (20 mL) was added pyridine (1.74 g, 24.5 mmol) at −78° C. The mixture was warmed to 0° C. and stirred for 20 min. The reaction volatiles were concentrated in vacuum to obtain ethenesulfonyl chloride as colorless oil, which was used directly without further purification.

To a solution of L-Lys(Boc)-OMe HCl (3.19 g, 12.25 mmol) and triethylamine (2.48 g, 12.25 mmol) in DCM (5 mL) was added a solution of ethenesulfonyl chloride (1.55 g, 12.25 mmol) in DCM (50 mL). The reaction mixture was stirred overnight at room temperature. The reaction volatiles were concentrated in vacuum and the residue was re-dissolved with EtOAc (100 mL), which was further washed with 0.5 N HCl (50 mL) and brine (50 mL) sequentially. The organic layer was dried and concentrated in vacuum. The resulting residue was further purified by silica gel chromatography (PE:EA=3:1) to obtained compound 245AA, a colorless oil (1.7 g, 39.6%).

To a solution of compound 245AA (1.5 g, 4.28 mmol) in MeOH (20 mL) was added decan-1-amine (733 mg, 1.24 mmol) dropwise at 0° C. The mixture was warmed to room temperature gradually and stirred overnight at room temperature. The reaction volatiles were concentrated in vacuum and the residue was then purified by silica gel chromatography (PE:EA=20:1 to 1:1) to obtain compound 245BB, a colorless oil (0.84 g, 38%). LC-MS (General Method 12): MS (ESI) m/z 522.3 (M+H)⁺; t_{R} 0.869 min.

The ester in compound 245BB (750 mg, 1.44 mmol) was hydrolyzed with LiOH in a manner similar to that described for compound 215C to afford compound 245A (708 mg, 97%) as a yellow solid.

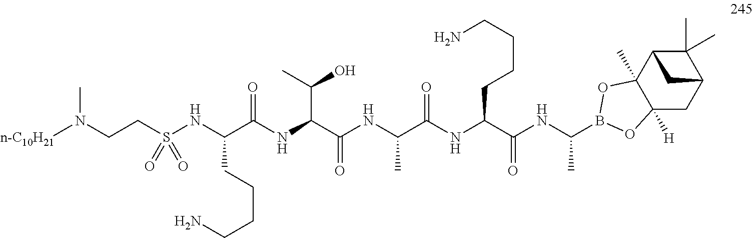

Compound 245 was prepared as the HCl salt using General Methods 1 and 13 from compound 245A and compound 101C2. LC-MS (General Method 12): MS (ESI) m/z 913.5 (M+H)⁺; t_{R} 0.792 min.

### Example 77

### Preparation of Compound 246

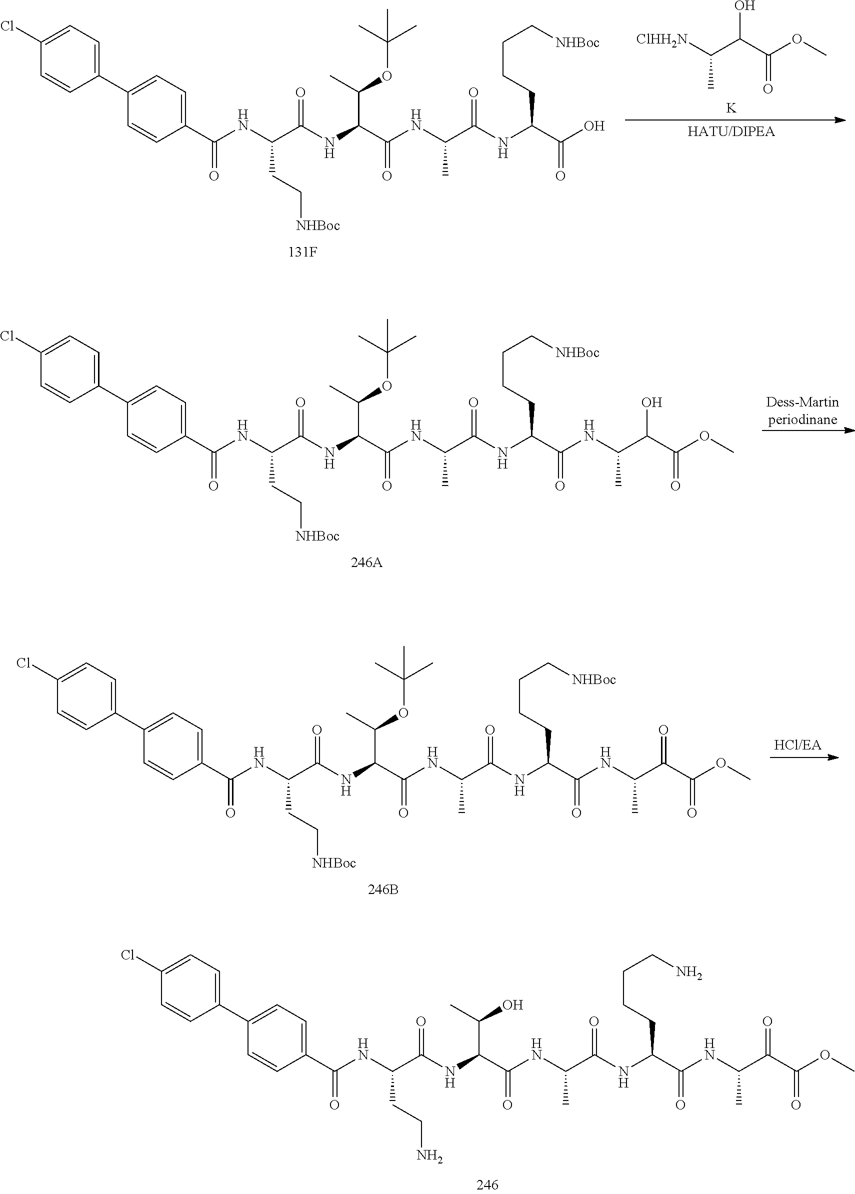

The peptide coupling was performed with HATU between compound K (180 mg, 1.1 mmol) and compound 131F (100 mg, 0.11 mmol) in a manner similar to that described for compound 101G, to obtain compound 246A as a yellow solid (98 mg, 88%).

To a solution of compound 246A (98 mg, 0.1 mmol) in DCM (20 mL) was added Dess-Martin periodinane (169 mg, 0.4 mmol) at 0° C. The reaction mixture was stirred at 25° C. for 16 h. After TLC showed the reaction was completed, the reaction mixture was poured into a saturated solution of NaHCO₃/NaS₂O₃ (30 mL) and the aqueous layer was extracted with DCM (20 mL×2). The combined organic layers were dried and concentrated under reduced pressure and the residue was purified by silica gel column (DCM:MeOH=50/140/1) to give compound 246B as a white solid (95 g, 98%).

To a solution of compound 246B (95 mg, 0.15 mmol) in EtOAc (2 mL) was added EA/HCl (1 mL) dropwise at 0° C. After LCMS showed reaction was completed, the reaction volatiles were concentrated and the residue was purified by HPLC (0.1% HCl) to give compound 246 (32 mg, 40%). LC-MS (General Method 12): MS (ESI) m/z 768.3 (M+Na)⁺; t_{R} 0.721 min.

### Example 78

### Preparation of Compound 247

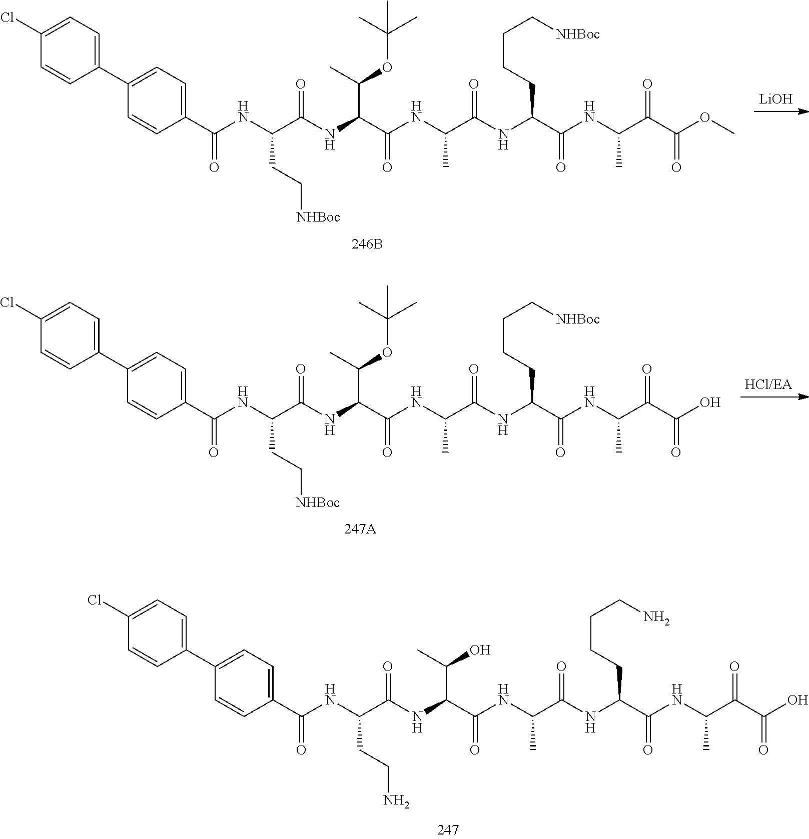

The ester in compound 246B (70 mg, 0.07 mmol) was hydrolyzed with LiOH in a manner similar to that described for compound 215C to afford compound 247A (60 mg, 87%) as a yellow solid.

The removal of the protecting groups in compound 247A (60 mg, 0.06 mmol) was performed using the HCl/EtOAc deprotection described for compound 246 to afford 10 mg (23%) of compound 247. LC-MS (General Method 12): MS (ESI) m/z 732.1 (M+H)⁺; t_{R} 0.688 min.

### Example 79

### Preparation of Compound 248

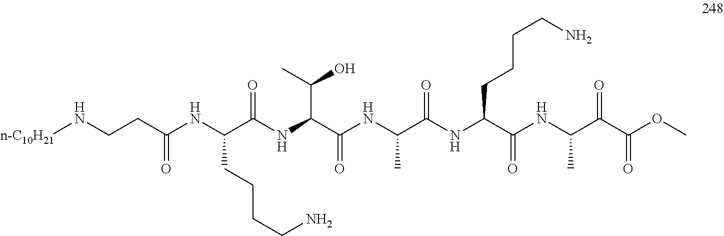

Compound 248 was prepared as the HCl salt in a manner similar to compound 246 utilizing the same peptide fragment used for compound 217. LC-MS (General Method 12): MS (ESI) m/z 771.5 (M+H)⁺; t_{R} 0.711 min

### Example 80

### Preparation of Compound 249

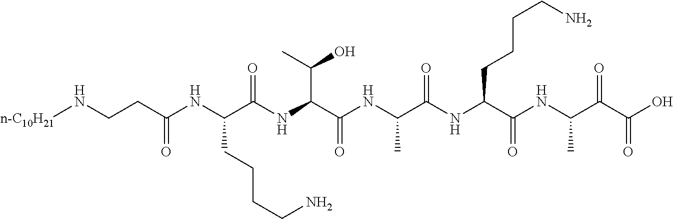

Compound 248 was prepared as the HCl salt by LiOH ester hydrolysis and protecting group removal with HCl in a manner similar to compound 247. LC-MS (General Method 12): MS (ESI) m/z 729.6 (M+H)⁺; t_{R} 0.728 min.

### Example 81

### Preparation of Compound 250

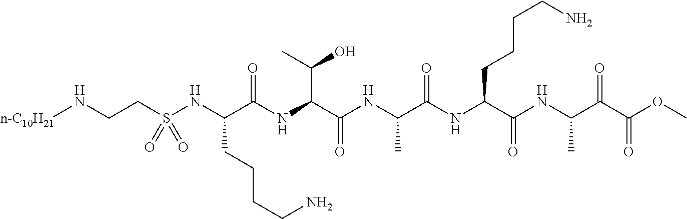

Compound 250 was prepared as the HCl salt in a manner similar to compound 246 utilizing the same peptide fragment used for compound 237. LC-MS (General Method 12): MS (ESI) m/z 807.5 (M+H)⁺; t_{R} 0.711 min.

### Example 82

### Preparation of Compound 251

Compound 251 was prepared as the HCl salt by LiOH ester hydrolysis and protecting group removal with HCl in a manner similar to compound 247. LC-MS (General Method 12): MS (ESI) m/z 793.5 (M+H)⁺; t_{R} 0.732 min.

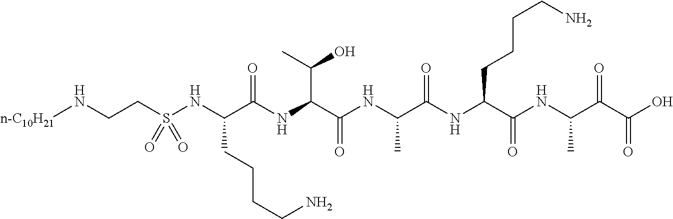

### Example 83

### Preparation of Compound 252

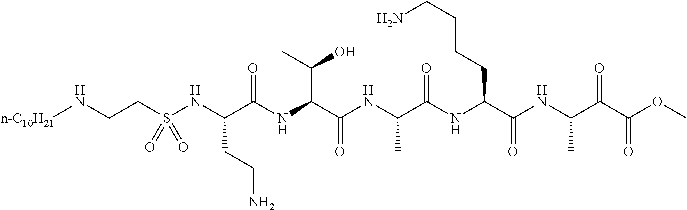

Compound 252 was prepared as the HCl salt in a manner similar to compound 246 utilizing the same peptide fragment used for compound 239. LC-MS (General Method 12): MS (ESI) m/z 779.5 (M+H)⁺; t_{R} 0.715 min.

### Example 84

### Preparation of Compound 253

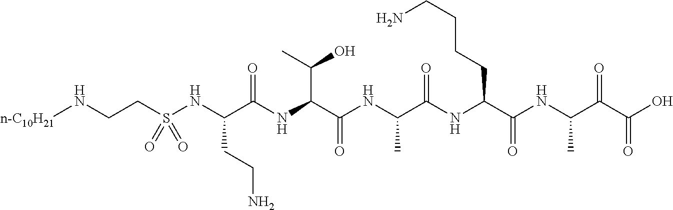

Compound 253 was prepared as the HCl salt by LiOH ester hydrolysis and protecting group removal with HCl in a manner similar to compound 247. LC-MS (General Method 12): MS (ESI) m/z 765.6 (M+H)⁺; t_{R} 0.723 min.

### Example 85

### Preparation of Compound 254

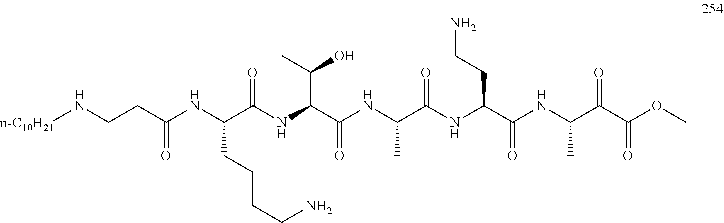

Compound 254 was prepared as the HCl salt in a manner similar to compound 246. LC-MS (General Method 12): MS (ESI) m/z 743.2 (M+H)⁺; t_{R} 0.719 min.

### Example 86

### Preparation of Compound 255

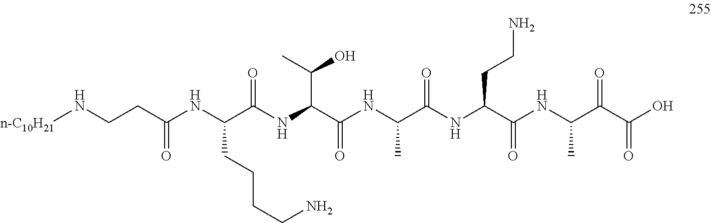

Compound 255 was prepared as the HCl salt by LiOH ester hydrolysis and protecting group removal with HCl in a manner similar to compound 247. LC-MS (General Method 12): MS (ESI) m/z 729.6 (M+H)⁺; t_{R} 0.728 min.

### Example 87

### Preparation of Compound 256

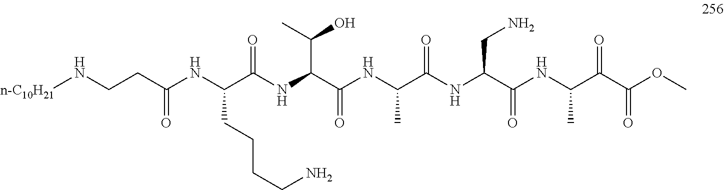

Compound 256 was prepared as the HCl salt in a manner similar to compound 246. LC-MS (General Method 12): MS (ESI) m/z 729.5 (M+H)⁺; t_{R} 0.723 min.

### Example 88

### Preparation of Compound 257

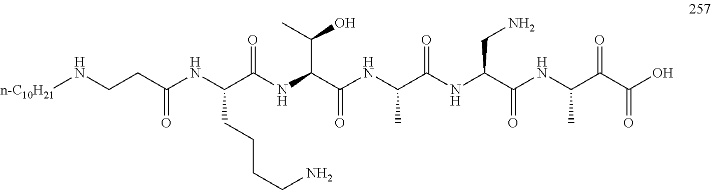

Compound 257 was prepared as the HCl salt by LiOH ester hydrolysis and protecting group removal with HCl in a manner similar to compound 247. LC-MS (General Method 12): MS (ESI) m/z 715.4 (M+H)⁺; t_{R} 0.715 min.

### Example 89

### Preparation of Compound 258

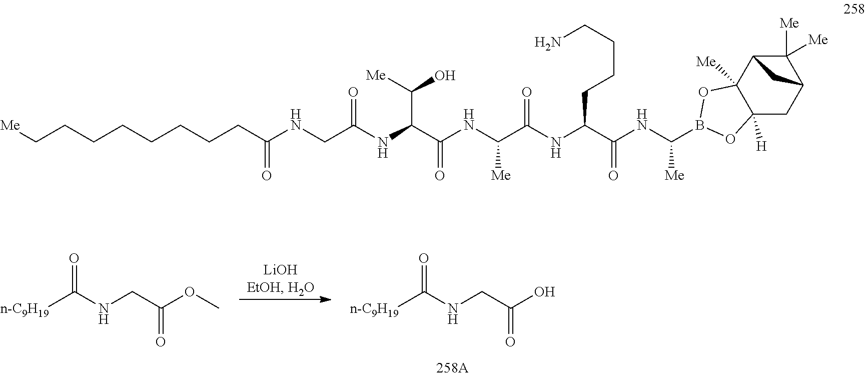

To a solution of methyl 2-decanamidoacetate (11.5 g, 47.2 mmol) in EtOH (110 mL) was added a solution of LiOH (3.92 g, 94.4 mmol) in H₂O (110 mL) at 0° C. The reaction mixture was allowed to warm to 30° C. and stirred for 18 h at the same temperature. After TLC showed that the reaction was completed, the reaction crude was evaporation to remove EtOH and the remaining aqueous was adjusted to pH=2˜3 with 6 N HCl, which was further extracted with EtOAc (50 mL*3). The combined EtOAc layers were dried over Na₂SO₄, and concentrated to give compound 258A as white solid (8.5 g, 78.5%).

Compound 258 was prepared as the HCl salt using General Methods 1 and 13 from compound 258A and compound 101C2. LC-MS (General Method 12): MS (ESI) m/z 734.5 (M+H)⁺; t_{R} 0.734 min.

### Example 90

### Preparation of Compound 259

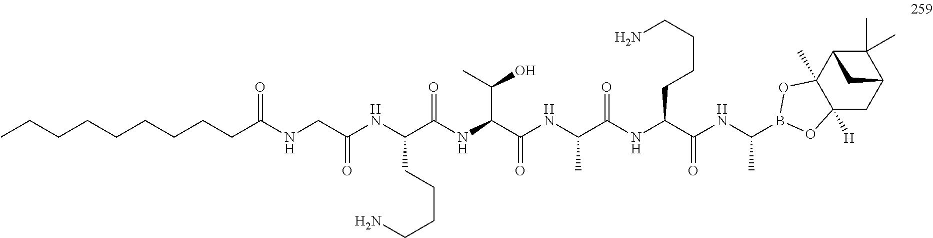 [SMILES: CCCCCCCCCC(=O)NCC(=O)N[C@@H](CCCCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 259 was prepared as the HCl salt using General Methods 1 and 13 from compound 258A and compound 126D2. LC-MS (General Method 12): MS (ESI) m/z 863.4 (M+H)⁺; t_{R} 0.799 min.

### Example 91

### Preparation of Compound 260

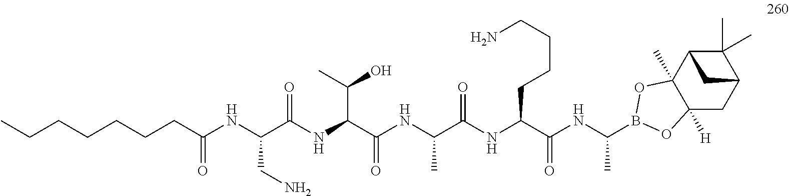 [SMILES: CCCCCCCC(=O)N[C@@H](CN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 260 was prepared as the HCl salt using General Methods 1 and 13 from compound 101D2. LC-MS (General Method 12): MS (ESI) m/z 736.2 (M−H₂O+H)⁺; t_{R} 0.757 min.

### Example 92

### Preparation of Compound 261

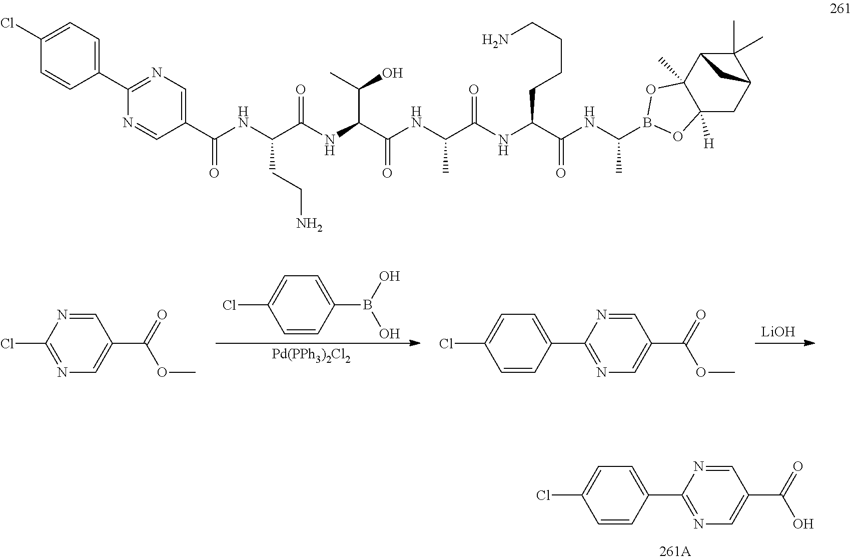

To a mixture of methyl 2-chloropyrimidine-5-carboxylate (1.81 g, 11.6 mmol) and compound (4-chlorophenyl)boronic acid (2.00 g, 11.6 mmol) in dioxane (100 mL) and water (20 mL) was added Pd(PPh₃)₂Cl₂ (200 mg) and Na₂CO₃ (2.46 g, 23.2 mmol). The reaction mixture was stirred under N₂ at 110° C. for 12 h. After TLC showed that the reaction was completed, the reaction volatiles were removed under reduced pressure and the residue was suspended in water (50 mL), which was further extracted with EA (50 mL×3). The combined organic layers were concentrated and the residue was purified by silica gel column (PE/EA=10:0˜10:1) to get the methyl 2-(4-chlorophenyl)pyrimidine-5-carboxylate (800 mg, 27.8%).

The ester hydrolysis of methyl 2-(4-chlorophenyl)pyrimidine-5-carboxylate was performed with LiOH in a manner similar to that described for compound 215C (800 mg, 3.22 mmol) to obtain 2-(4-chlorophenyl)pyrimidine-5-carboxylic acid (261A, 400 mg, 53%).

Compound 261 was prepared as the HCl salt using General Methods 1 and 13 from compound 261A. LC-MS (General Method 12): MS (ESI) m/z 840 (M−H₂O+H)⁺; t_{R} 0.759 min.

### Example 93

### Preparation of Compound 262

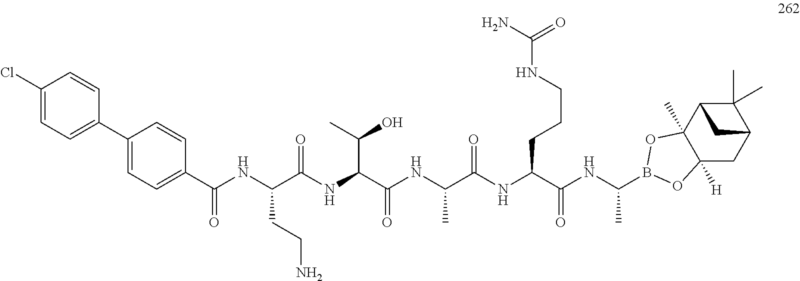 [SMILES: C[C@H](NC(=O)[C@H](CCCNC(N)=O)NC(=O)[C@H](C)NC(=O)[C@@H](NC(=O)[C@H](CCN)NC(=O)c1ccc(-c2ccc(Cl)cc2)cc1)[C@@H](C)O)B1O[C@@H]2C[C@@H]3C[C@@H](C3(C)C)[C@]2(C)O1]

Compound 262 was prepared as the HCl salt using General Methods 1 and 13. LC-MS for C₃₂H₄₆BClN₈O₉ (ESI) m/z 715.14 (M−H₂O+H)⁺; t_{R} 0.381 min (3% CH₃CN/H₂O, 0.3 min; 3% CH₃CN−95% CH₃CN/H₂O with 0.05% TFA, 6.5 min, 0.4 mL/min, Agilent SB C18, 2.1×30 mm).

### Example 94

### Preparation of Compound 263

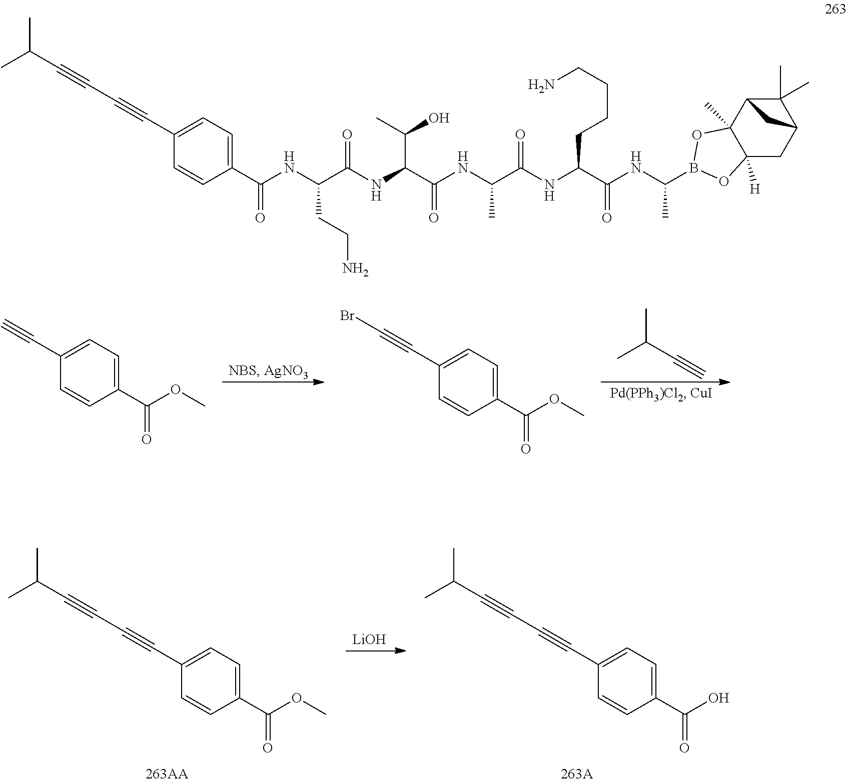

To a solution of methyl 4-ethynylbenzoate (9.6 g, 60 mmol) in acetone (100 mL) was added NBS (12.8 g, 72 mmol) and AgNO₃ (510 mg, 3 mmol) at room temperature. The mixture was stirred at room temperature for 4 h. After LCMS showed that the reaction was completed, the reaction volatiles were removed under reduced pressure and the residue was re-suspended with water (100 mL), which was extracted with EtOAc (100 mL×3). The combined organic layers were dried and concentrated. The residue was purified by silica gel column (PE:EA=20:1) to give methyl 4-(bromoethynyl)benzoate as a yellow solid (14 g, 98%).

To a solution of methyl 4-(bromoethynyl)benzoate (3.0 g, 12.6 mmol), 3-methylbut-1-yne (857 mg, 12.6 mmol), DIPEA (12 mL) in toluene was added Pd(PPh₃)₂Cl₂ (1.68 g, 2.5 mmol), CuI (950 mg, 5.0 mmol) under Ar (Sonogashira coupling). The reaction mixture was stirred at room temperature for 4 h. After TLC showed the reaction was completed, the reaction volatiles were concentrated under reduced pressure and the residue was purified by silica gel column (PE:EA=10:1) to give compound 263AA as a yellow solid (1.0 g, 36%).

The ester hydrolysis of compound 263AA (1.0 g, 4.4 mmol) was performed with LiOH in a manner similar to that described for compound 215C to afford compound 263A as a light yellow solid (900 mg, 97%).

Compound 263 was prepared as the HCl salt using General Methods 1 and 13 from compound 263A. LC-MS (General Method 12): MS (ESI) m/z 818.6 (M−H₂O+H)⁺; t_{R} 0.789 min.

### Example 95

### Preparation of Compound 264

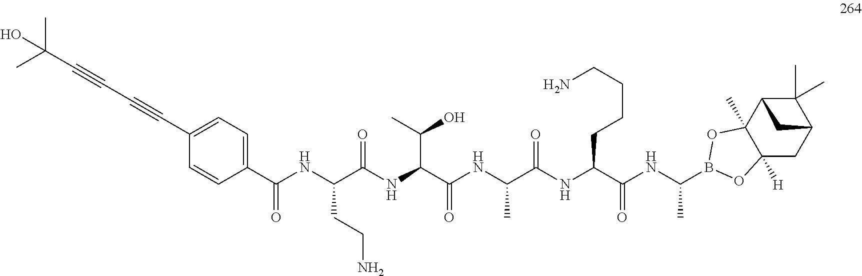 [SMILES: C[C@H](NC(=O)[C@H](CCCCN)NC(=O)[C@H](C)NC(=O)[C@@H](NC(=O)[C@H](CCN)NC(=O)c1ccc(C#CC#CC(C)(C)O)cc1)[C@@H](C)O)B1O[C@@H]2C[C@@H]3C[C@@H](C3(C)C)[C@]2(C)O1]

Compound 264 was prepared as the HCl salt using General Methods 1 and 13 in a manner similar to compound 263 except 2-methylbut-3-yn-2-ol is used as the starting material in the Sonogashira coupling. LC-MS (General Method 12): MS (ESI) m/z 834.5 (M−H₂O+H)⁺; t_{R} 0.744 min.

### Example 96

### Preparation of Compound 265

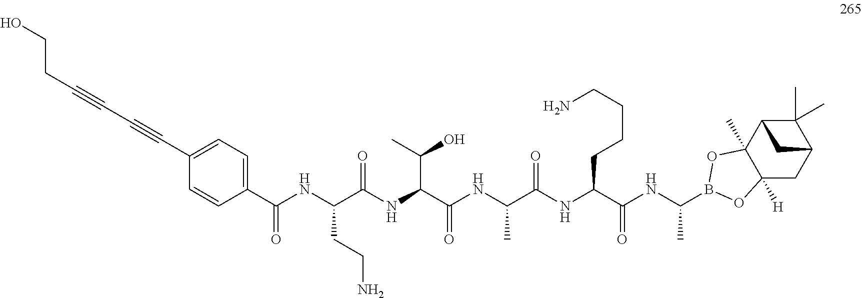 [SMILES: C[C@H](NC(=O)[C@H](CCCCN)NC(=O)[C@H](C)NC(=O)[C@@H](NC(=O)[C@H](CCN)NC(=O)c1ccc(C#CC#CCCO)cc1)[C@@H](C)O)B1O[C@@H]2C[C@@H]3C[C@@H](C3(C)C)[C@]2(C)O1]

Compound 265 was prepared as the HCl salt using General Methods 1 and 13 in a manner similar to compound 263 except but-3-yn-1-ol is used as the starting material in the Sonogashira coupling. LC-MS (General Method 12): MS (ESI) m/z 821.5 (M+H)⁺; t_{R} 0.700 min.

### Example 97

### Preparation of Compound 266

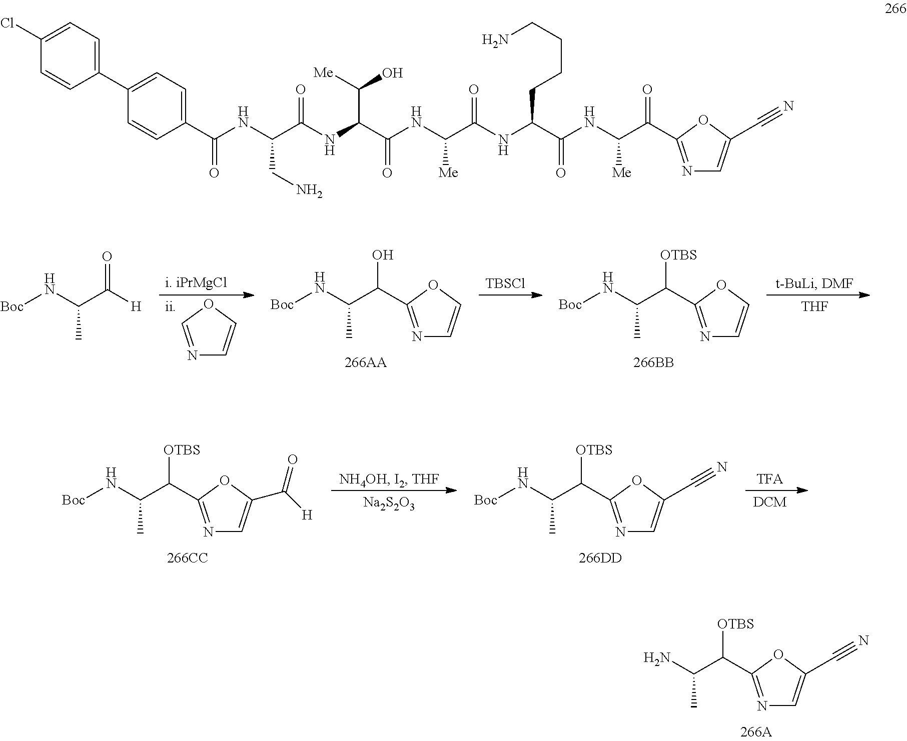

To a solution of oxazole (5.98 g, 86.6 mmol) in toluene (50 mL) was added 2 M i-PrMgCl in THF (43.3 mL, 86.6 mmol) at 0° C., followed by the addition of Boc-L-alanine aldehyde (10 g, 57.7 mmol) in THF (100 mL) at 0° C. The mixture was stirred for 1 h at 0° C. and 3 h at room temperature until TLC showed the completion of the reaction. The reaction mixture was quenched with 5% NaHCO₃ (100 mL), which was extracted by ethyl acetate (100 mL×3). The combined organic layers were washed with brine (300 mL), dried over Na₂SO₄ and concentrated under reduced pressure; and the residue was purified by silica gel column (PE:EA=1/1) to give compound 266AA as yellow oil (5.87 g, 42%).

To a mixture of compound 266AA (5.2 g, 21.5 mmol), imidazole (4.32 g, 64.4 mmol) and DMAP (0.52 g, 4.29 mmol) in DCM (50 mL) was added TBSCl (3.9 g, 25.8 mmol) at 0° C. The mixture was stirred overnight at room temperature. After that, to the reaction mixture was added saturated NH₄Cl (40 mL), and the aqueous layer was further extracted by DCM (50 mL×2). The combined organic layers were washed with brine (150 mL), dried over Na₂SO₄ and concentrated under reduced pressure; and the residue was purified by silica gel column (PE/EA=10:1) to give compound 266BB as yellow oil (4.7 g, 62%).

To a solution of compound 266BB (4.1 g, 11.5 mmol) in THF (50 mL) was added t-BuLi (13.3 mL, 17.2 mmol) dropwise at −78° C. The mixture was stirred for 2 h at −78° C., followed by the addition of a solution of DMF (4.2 g, 57.5 mmol) in anhydrous THF (20 mL). After TLC showed the reaction was completed, the mixture was quenched with saturated NH₄Cl (40 mL) carefully, where the aqueous layer was further extracted by EtOAc (50 mL×3). The combined organic layers were washed with brine (200 mL), dried over Na₂SO₄ and concentrated under reduced pressure; and the residue was purified by silica gel column (PE/EA=5:1) to give compound 266CC as colorless oil (2.1 g, 47.5%).

To a solution of compound 266CC (4.1 g, 11.5 mmol) in NH₄OH (30 mL) was added I₂ (1.58 g, 6.24 mmol) in portions at 0° C. The reaction mixture was stirred for 2 h at room temperature, then saturated Na₂S₂O₃ (10 mL) was added to the reaction and the mixture was stirred overnight. After that, the reaction mixture was extracted by EtOAc (50 mL×3). The combined organic layers were washed with brine (50 mL), dried over Na₂SO₄ and concentrated under reduced pressure; and the residue was purified by silica gel column (PE/EA=10:1) to give compound 266DD as a colorless oil (1.2 g, 61.5%). ¹H NMR (400 MHz, CDCl₃) δ 7.67 (1H, s), 4.83-4.92 (1H, m), 4.64 (1H, br), 4.08 (1H, s, br), 1.41-1.42 (9H, m), 1.23 (1H, d, J=6.8 Hz), 1.12 (2H, d, J=6.8 Hz), 0.91 (s, 9H), 0.08 (s, 3H), 0.01 (s, 3H).

To a solution of compound 266DD (320 mg, 0.84 mmol) in DCM (6 mL) was added TFA (2 mL). The reaction mixture was stirred at room temperature for 3 h until TLC showed the completion of the reaction. Reaction volatiles were concentrated to afford the compound 266A, which was used in next step without purification.

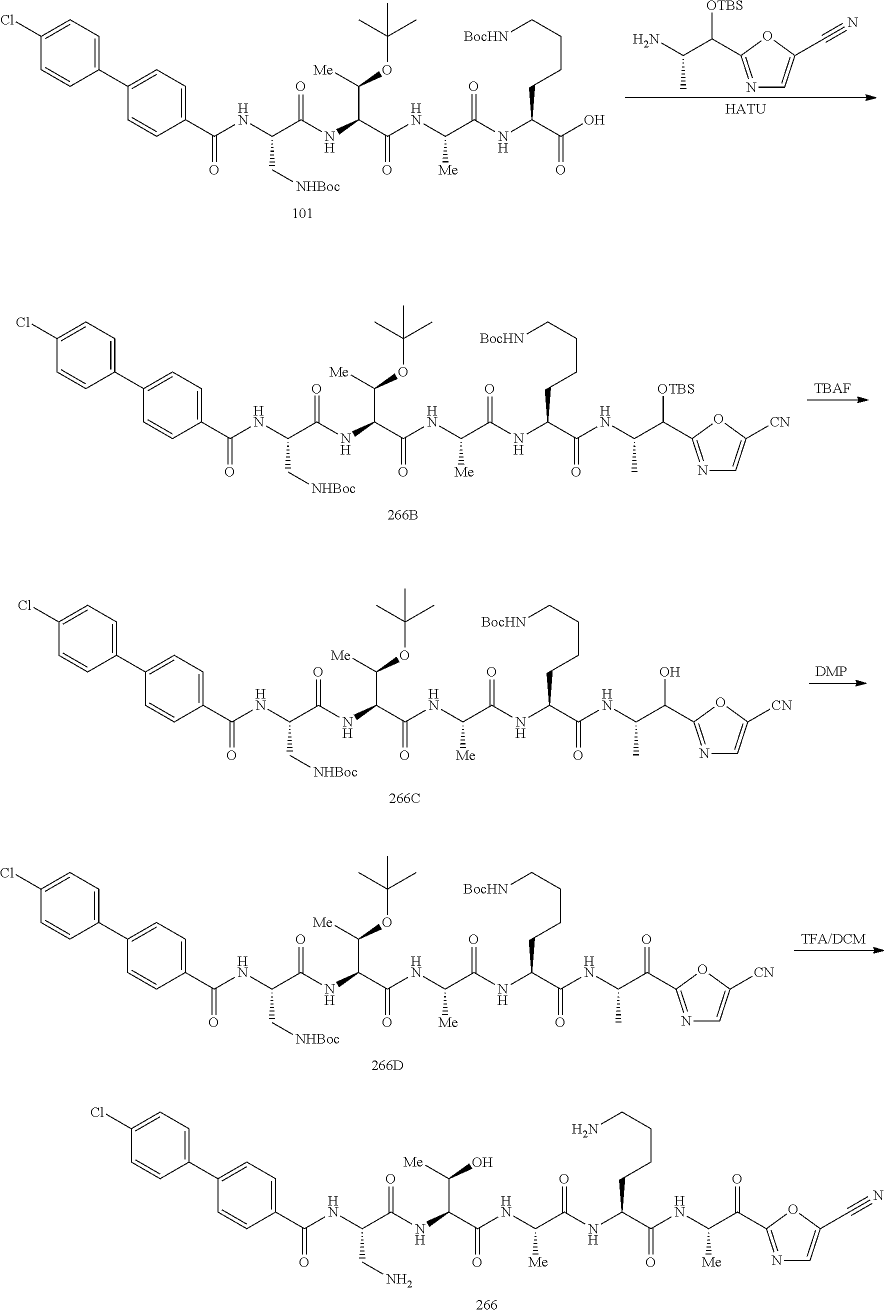

The peptide coupling was performed with HATU between compound 101F (300 mg, 0.342 mmol) and compound 266A (193 mg, 0.685 mmol) in a manner similar to that described for compound 101G, to obtain compound 266B as a white solid (281 mg, 72%).

To a solution of compound 266B (280 mg, 0.375 mmol) in DCM (20 mL) was added a solution of TBAF (129 mg, 0.491 mmol) in THF (10 mL) at 0° C. The reaction mixture was stirred for 2 h and then quenched with saturated aqueous NH₄Cl (20 mL), in which the aqueous layer was further extracted with DCM (30 mL×2). The combined organic layers were washed with brine (10 mL), dried over Na₂SO₄, and concentrated under reduced pressure; and the residue was purified by silica gel column (DCM/MeOH=20:1) to give compound 266C as colorless oil (181 mg, 72.1%).

Compound 266C (181 mg, 0.176 mmol) was subjected to Dess-Martin periodinane oxidation in a manner similar to the preparation of compound 246B to afford compound 266D (135 mg, 75%).

To a solution of compound 266D (135 mg, 0.132 mmol) in DCM (6 mL) was added TFA (2 mL), and the reaction mixture was stirred at room temperature for 3 h until TLC showed the completion of the reaction. The reaction volatiles were removed under reduced pressure and the residue was purified by a reverse-phase preparatory HPLC (0.1% formic acid) to afford compound 266 as a white solid (62 mg, 60.4%). LC-MS (General Method 12): MS (ESI) m/z 766.1 (M+H)⁺; t_{R} 0.740 min.

### Example 98

### Preparation of Compound 267

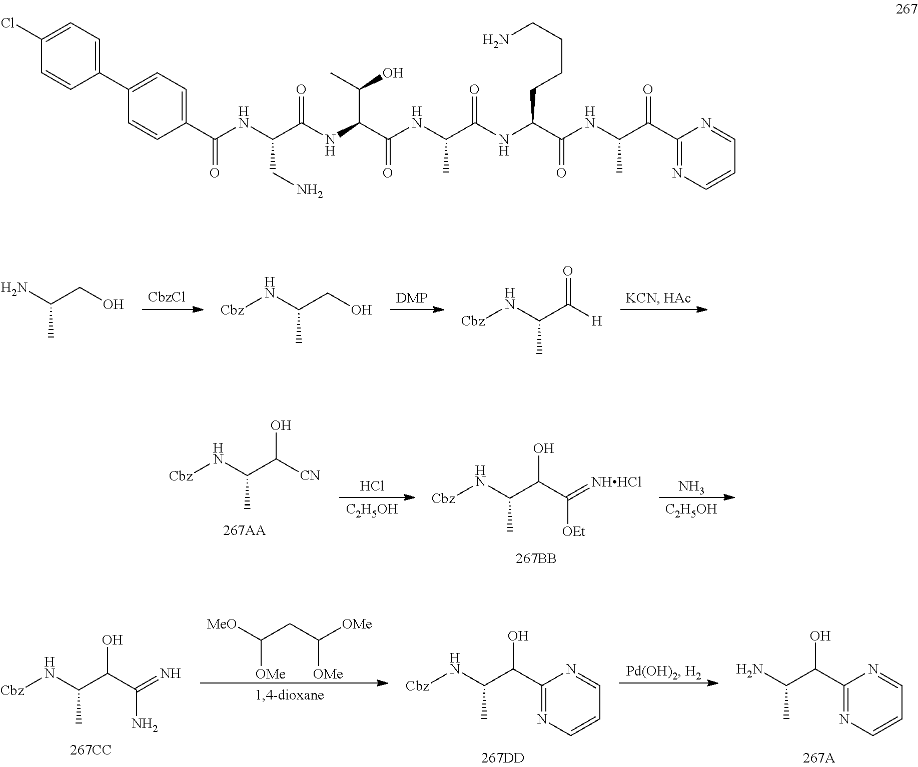

To a solution of (S)-2-aminopropan-1-ol (10 g, 133 mmol) and DIPEA (34.4 g, 266 mmol) in DCM (300 mL) was added CbzCl (22.7 g, 133 mmol) in DCM (100 mL) dropwise at 0° C., the reaction mixture was stirred at 30° C. for 2 h. After TLC showed that the reaction was completed, the reaction mixture was washed with water (500 mL) and brine (500 mL). The organic layer was dried and concentrated under reduced pressure; and the residue was purified by silica gel column (PE:EtOAc=20/110/1) to give (S)-benzyl (1-hydroxypropan-2-yl)carbamate as a white solid (19.8 g, 71%).

To a mixture of (S)-benzyl (1-hydroxypropan-2-yl)carbamate (22 g, 105 mmol) in DMF (50 mL) was added solid NaHCO₃ (177 g, 2.1 mol); and then DMP (67 g, 158 mmol) was added to the solution at 0° C. The reaction was stirred at 30° C. for 2 h. After TLC showed that the reaction was completed, the reaction mixture was poured into a saturated solution of NaHCO₃/NaS₂O₃. The aqueous phase was extracted with DCM (500 mL×3). The combined organic layers were dried over Na₂SO₄ and concentrated under reduced pressure; and the residue was purified by silica gel column (PE:EtOAc=20/115/1) to give (S)-benzyl (1-oxopropan-2-yl)carbamate as a colorless oil (21.7 g, 93%).

To a mixture of (S)-benzyl (1-oxopropan-2-yl)carbamate (16 g, 77.2 mmol) in EA (90 mL)/MeOH (90 mL) was added solid KCN (5.7 g, 87 mmol), followed by the addition AcOH (4.6 g, 87 mmol) at 0° C. The reaction was stirred at 30° C. for 16 h. After TLC showed that the reaction was completed, reaction volatiles were removed under reduced pressure and the residue was poured into water (100 mL), which was extracted with EA (100 mL×3). The combined organic layers were dried over Na₂SO₄ and concentrated under reduced pressure; and the residue was purified by silica gel column (PE:EtOAc=10/16/1) to give compound 267AA as colorless oil (14 g, 61.4%).

A mixture of compound 267AA (14 g, 59.7 mmol) in C₂H₅OH (30 mL) was stirred at −40° C. for 5 min. HCl gas was then passed into the solution for 12 min. The reaction was stirred at 0° C. for another 50 min. After that, reaction volatiles were removed under reduced pressure to obtain compound 267BB without further purification. The crude was re-dissolved in C₂H₅OH (40 mL) and the mixture was stirred at −40° C. for 5 min. NH₃ gas was then passed into the solution for 12 min. The reaction mixture was stirred at 30° C. for 16 h. After that, reaction volatiles were removed under reduced pressure to obtain compound 267CC (15.0 g) without further purification.

A mixture of 1,1,3,3-tetramethoxypropane (9.79 g, 59.7 mmol) in 1,4-dioxane (20 mL) was added EtOAc/HCl (5 mL) at 30° C. for 30 min. Et₃N (10 mL) was then added dropwise at 0° C. for 15 min, followed by the addition of compound 267CC (15.0 g, 59.7 mmol). The reaction mixture was stirred at 80° C. for 16 h. After TLC showed that the reaction was completed, reaction volatiles were removed under reduced pressure and the residue was poured into water (100 mL), which was extracted with DCM (100 mL×3). The combined organic layers were dried over Na₂SO₄ and concentrated under reduced pressure; and the residue was purified by silica gel column (PE:EtOAc=6/13/1) to give compound 267DD as colorless oil (700 mg, 4%).

To a mixture of compound 267DD (220 mg, 0.76 mmol) in MeOH (20 mL) was added Pd(OH)₂ (70 mg), and the solution was stirred at 30° C. under H₂ for 5 min, in which the reaction needs to be carefully monitored to avoid over reduction. After that, the reaction mixture was filtered and the volatiles were removed under reduced pressure to obtain compound 267A (106 mg) without further purification.

Compound 267 was prepared as the formic acid salt from compound 267A (HATU peptide coupling, Dess-Martin periodinane oxidation, and TFA hydrolysis) in a manner similar to compound 266 to afford compound 267. LC-MS (General Method 12): MS (ESI) m/z 752.1 (M−H₂O+H)⁺; t_{R} O. 711 min.

### Example 99

### Preparation of Compounds 268 and 269

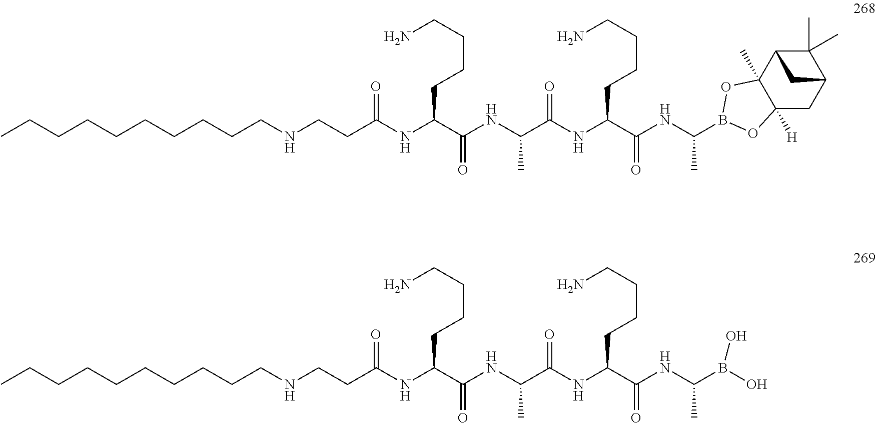

Compounds 268 and 269 were prepared using General Methods 1 and 13 from Compound 217A, and both compounds are isolated during the preparative HPLC separation (C18, CH₃CN/H₂O plus 0.05% HCl).

Analytical data for Compound 268: MS (ESI) m/z 762.5 (M+H)⁺; t_{R} 1.79 min (10% CH₃CN/H₂O−80% CH₃CN/H₂O, 4 min, 0.8 mL/min, Xtimate C18, 2.1×30 mm).

Ok Analytical data for Compound 269: MS (ESI) m/z 305.6 ((M-18)/2+H)⁺; t_{R} 1.28 min (10% CH₃CN/H₂O−80% CH₃CN/H₂O, 4 min, 0.8 mL/min, Xtimate C18, 2.1×30 mm).

### Example 100

### Preparation of Compound 270

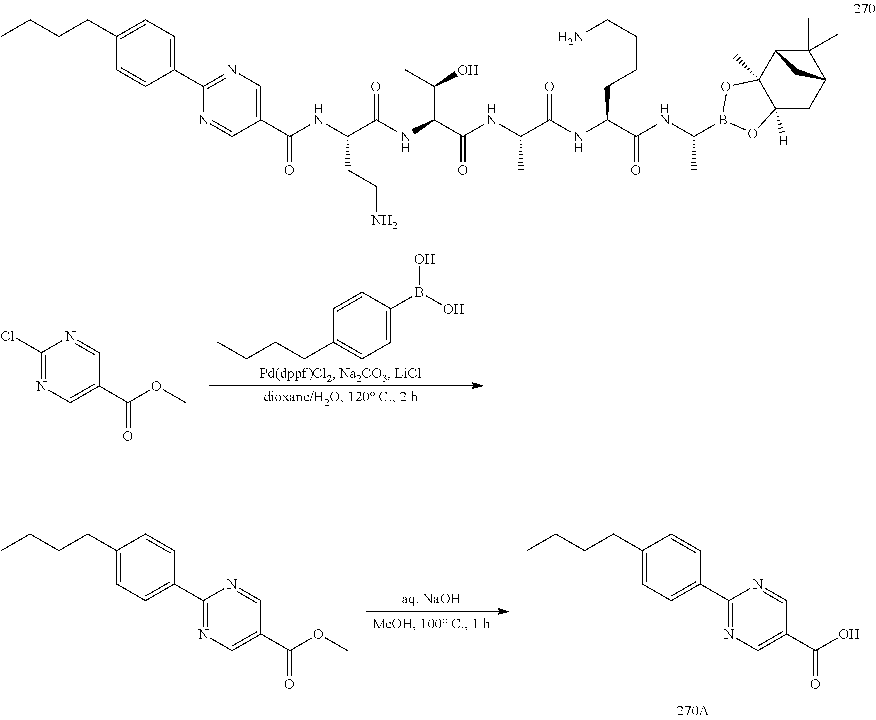

A mixture of methyl 2-chloropyrimidine-5-carboxylate (170 mg, 0.99 mmol), (4-butylphenyl)boronic acid (176 mg, 0.99 mmol), Pd(dppf)Cl₂ (36 mg, 0.05 mmol), sodium carbonate (577 mg, 5.45 mmol) and lithium chloride (21 mg, 0.50 mmol) in 1,4-dioxane/water (8 mL, 3:1) was stirred at 120° C. for 2 h under nitrogen atmosphere. The reaction was quenched with water (10 mL). The mixture was extracted with ethyl acetate (3×20 mL). The combined extracts were washed with brine (2×20 ml), dried over anhydrous Na₂SO₄ and filtered. The filtrate was concentrated and the residue was purified by column chromatography on silica gel (eluting with petroleum ether/ethyl acetate from 100:1 to 10:1) to afford methyl 2-(4-butylphenyl)pyrimidine-5-carboxylate (140 mg, 52%) as a yellow oil. MS-ESI: [M+H]⁺=270.9. ¹H NMR (400 MHz, CDCl₃) δ 9.30 (s, 2H), 8.43 (d, J=8.4 Hz, 2H), 7.34 (d, J=8.0 Hz, 2H), 4.00 (s, 3H), 2.70 (t, J=8.0 Hz, 2H), 1.68-1.64 (m, 2H), 1.42-1.36 (m, 2H), 0.95 (t, J=7.6 Hz, 3H).

To a solution of methyl 2-(4-butylphenyl)pyrimidine-5-carboxylate (0.32 g, 1.19 mmol) in methanol (10 mL) was added aqueous sodium hydroxide (10 mL, 50 mmol, 5.0 M). The reaction mixture was stirred at 100° C. for 2 h. The reaction was cooled at 20° C. and hydrochloride acid (1.0 M) was added until pH=3-4. The mixture was extracted with ethyl acetate (3×50 mL). The combined extracts were washed with brine (2×50 mL), dried over sodium sulfate and concentrated to give Compound 270A (370 mg, crude). MS-ESI: [M+H]⁺=257.3.

Compound 270 was prepared using General Methods 1 and 13 from Compound 270A and is obtained upon prep-HPLC purification (C18, CH₃CN/H₂O plus 0.05% HCl).

Analytical data for Compound 270: MS (ESI) m/z 431.8 (M/2+H)⁺; t_{R} 1.89 min (10% CH₃CN/H₂O−80% CH₃CN/H₂O, 4 min, 0.8 mL/min, Xtimate C18, 2.1×30 mm).

### Example 101

### Preparation of Compound 271

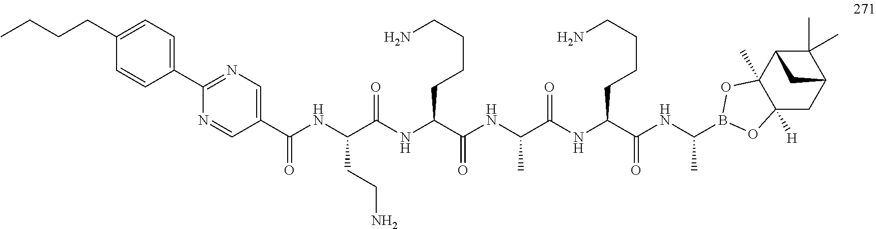 [SMILES: CCCCc1ccc(-c2ncc(C(=O)N[C@@H](CCN)C(=O)N[C@@H](CCCCN)C(=O)N[C@@H](C)C(=O)N[C@@H](CCCCN)C(=O)N[C@@H](C)B3O[C@@H]4C[C@@H]5C[C@@H](C5(C)C)[C@]4(C)O3)cn2)cc1]

Compound 271 was prepared using General Methods 1 and 13 from Compound 270A and is obtained upon prep-HPLC purification (C18, CH₃CN/H₂O plus 0.05% HCl).

Analytical data for Compound 271: MS (ESI) m/z 445.3 (M/2+H)⁺; t_{R} 1.71 min (10% CH₃CN/H₂O−80% CH₃CN/H₂O, 4 min, 0.8 mL/min, Xtimate C18, 2.1×30 mm).

### Example 102

### Preparation of Compound 272

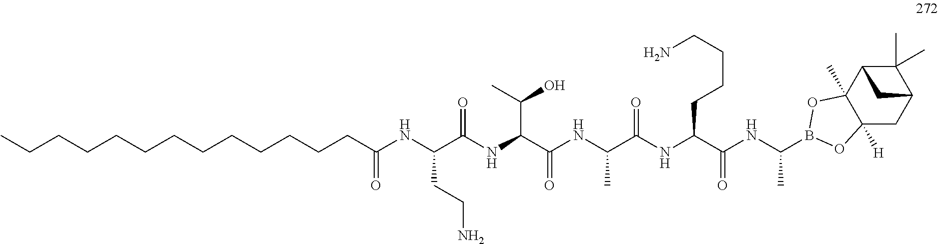 [SMILES: CCCCCCCCCCCCCC(=O)N[C@@H](CCN)C(=O)N[C@H](C(=O)N[C@@H](C)C(=O)N[C@@H](CCCCN)C(=O)N[C@@H](C)B1O[C@@H]2C[C@@H]3C[C@@H](C3(C)C)[C@]2(C)O1)[C@@H](C)O]

Compound 272 was prepared using General Methods 1 and 13 from commercial available tetradecanoic acid and is obtained upon prep-HPLC purification (C18, CH₃CN/H₂O plus 0.05% HCl).

Analytical data for Compound 272: MS (ESI) m/z 417.8 (M/2+H)⁺; t_{R} 2.23 min (10% CH₃CN/H₂O−80% CH₃CN/H₂O, 4 min, 0.8 mL/min, Xtimate C18, 2.1×30 mm).

### Example 103

### Preparation of Compound 273

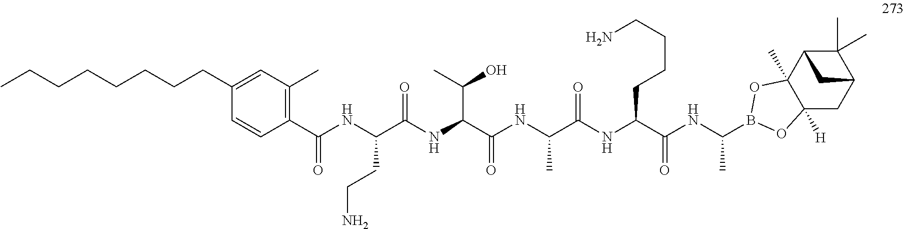 [SMILES: CCCCCCCCc1ccc(C(=O)N[C@@H](CCN)C(=O)N[C@H](C(=O)N[C@@H](C)C(=O)N[C@@H](CCCCN)C(=O)N[C@@H](C)B2O[C@@H]3C[C@@H]4C[C@@H](C4(C)C)[C@]3(C)O2)[C@@H](C)O)c(C)c1]

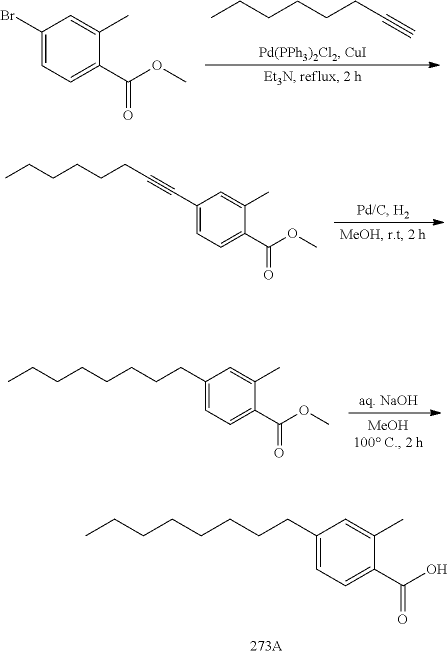

A mixture of methyl 4-bromo-2-methylbenzoate (1.0 g, 4.39 mmol), oct-1-yne (0.44 g, 3.99 mmol), Pd(PPh₃)₂Cl₂ (140 mg, 0.20 mmol) and CuI (38 mg, 0.20 mmol) in triethylamine (20 mL) was stirred at 100° C. for 2 h under nitrogen atmosphere. The reaction was quenched with water (30 mL). The mixture was extracted with dichloromethane (3×50 mL). The combined extracts were washed with brine (2×50 mL), dried over anhydrous Na₂SO₄ and concentrated. The residue was purified by column chromatography on silica gel (eluting with petroleum ether/ethyl acetate from 100:1 to 10:1) to afford methyl 2-methyl-4-(oct-1-yn-1-yl)benzoate (1.13 g, 100%) as a yellow oil.

A mixture of methyl 2-methyl-4-(oct-1-yn-1-yl)benzoate (1.13 g, 4.37 mmol) and Pd/C (0.2 g) in methanol (20 mL) was stirred at 25° C. for 16 h under hydrogen atmosphere. The catalyst was filtered off and the solvent was evaporated. The residue was purified by column chromatography on silica gel (eluting with petroleum ether/ethyl acetate from 100:1 to 10:1) to afford methyl 2-methyl-4-octylbenzoate (0.97 g, 84%) as a yellow oil.

To a solution of methyl 2-methyl-4-octylbenzoate (0.97 g, 3.70 mmol) in methanol (10 mL) was added aqueous sodium hydroxide (10 mL, 50 mmol, 5.0 M). The reaction mixture was stirred at 100° C. for 2 h. The reaction was cooled at 20° C. and hydrochloric acid (1.0 M) was added until pH=3-4. The mixture was extracted with ethyl acetate (3×50 mL). The combined extracts were washed with brine (2×50 mL), dried over sodium sulfate and filtered. The filtrate was concentrated to give Compound 273A (890 mg, 97%) as a yellow oil. ¹H NMR (400 MHz, CDCl₃): δ 7.98 (d, J=8.8 Hz, 1H), 7.10-7.08 (m, 2H), 2.64-2.60 (m, 5H), 1.35-1.20 (m, 12H), 0.89 (t, J=6.8 Hz, 3H).

Compound 273 was prepared using General Methods 1 and 13 from Compound 273A, and is obtained upon prep-HPLC purification (C18, CH₃CN/H₂O plus 0.05% HCl).

Analytical data for Compound 273: MS (ESI) m/z 427.7 (M/2+H)⁺; t_{R} 2.14 min (10% CH₃CN/H₂O−80% CH₃CN/H₂O, 4 min, 0.8 mL/min, Xtimate C18, 2.1×30 mm).

### Example 104

### Preparation of Compound 274

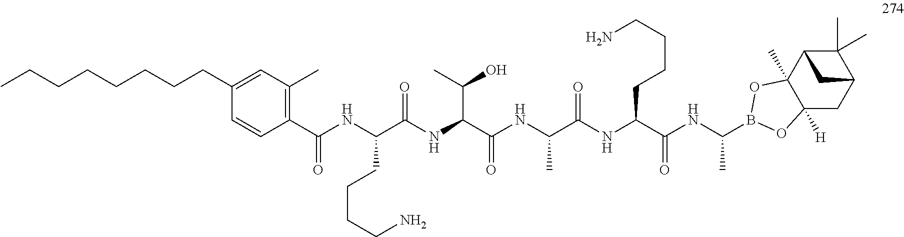 [SMILES: CCCCCCCCc1ccc(C(=O)N[C@@H](CCCCN)C(=O)N[C@H](C(=O)N[C@@H](C)C(=O)N[C@@H](CCCCN)C(=O)N[C@@H](C)B2O[C@@H]3C[C@@H]4C[C@@H](C4(C)C)[C@]3(C)O2)[C@@H](C)O)c(C)c1]

Compound 274 was prepared using General Methods 1 and 13 from Compound 273A, and is obtained upon prep-HPLC purification (C18, CH₃CN/H₂O plus 0.05% HCl).

Analytical data for Compound 274: MS (ESI) m/z 441.9 (M/2+H)⁺; t_{R} 2.18 min (10% CH₃CN/H₂O−80% CH₃CN/H₂O, 4 min, 0.8 mL/min, Xtimate C18, 2.1×30 mm).

### Example 105

### Preparation of Compounds 275 and 276 (derived from Compound 275 P1)

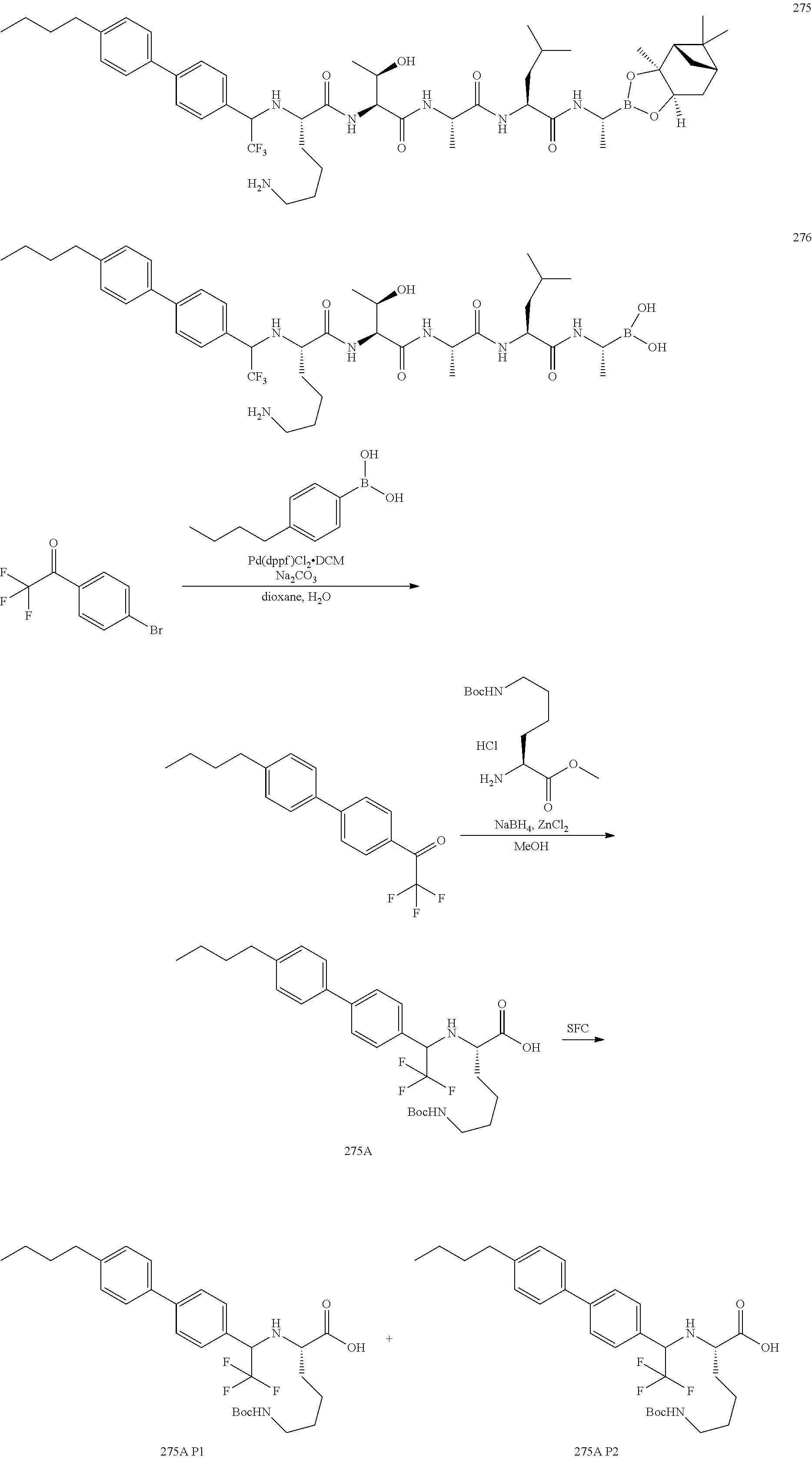

A mixture of (4-butylphenyl)boronic acid (500 mg, 2.8 mmol), 1-(4-bromophenyl)-2,2,2-trifluoroethanone (705 mg, 2.8 mmol), Pd(dppf)Cl₂-DCM (122.5 mg, 0.14 mmol), sodium carbonate (1.63 g, 15.4 mmol) and lithium chloride (411.6 mg, 9.8 mmol) in 1,4-dioxane/water (12 mL, 5:1) was stirred at 80° C. for 1 h under nitrogen atmosphere. The reaction was quenched with water (15 mL). The mixture was extracted with ethyl acetate (3×15 mL). The combined extracts were washed with brine (2×10 mL), dried over anhydrous sodium sulfate and filtered. The filtrate was concentrated and the residue was purified by column chromatography on silica gel (eluting with petroleum ether/ethyl acetate 5:1) to afford 1-(4′-butyl-[1,1′-biphenyl]-4-yl)-2,2,2-trifluoroethanone as a yellow oil (500 mg, 58%). ¹H NMR (400 MHz, CDCl₃): δ 8.15 (d, J=8.4 Hz, 2H), 7.77 (d, J=8.4 Hz, 2H), 7.59 (d, J=8.0 Hz, 2H), 7.33 (d, J=8.0 Hz, 2H), 2.70 (t, J=7.6 Hz, 2H), 1.70-1.62 (m, 2H), 1.44-1.38 (m, 2H), 0.97 (t, J=7.4 Hz, 3H).

To a solution of 1-(4′-butyl-[1,1′-biphenyl]-4-yl)-2,2,2-trifluoroethanone (500 mg, 1.6 mmol) and (S)-methyl 2-amino-6-((tert-butoxycarbonyl)amino)hexanoate hydrochloride (484 mg, 1.6 mmol) in methanol (10 mL) was added potassium carbonate (662 mg, 4.8 mmol). The reaction was stirred at 50° C. for 5 h and cooled to 25° C. for 16 h. To another flask was added zinc chloride (1 M solution in ether, 4 mL) and sodium borohydride (122 mg, 3.2 mmol). The solution was stirred at 25° C. for 16 h and was added to the former reaction mixture at −45° C. Then reaction was stirred at 0° C. for 1 h. TLC (petroleum ether/ethyl acetate=1:1) showed that starting material was consumed completely. The solution was poured into hydrochloride acid (1 M, 4 mL) at 0° C. The mixture was extracted with ethyl acetate (3×15 mL). The combined extracts were washed with brine (2×10 mL), dried over anhydrous sodium sulfate and filtered. The filtrate was concentrated and the residue was purified by column chromatography on silica gel (eluting with petroleum ether/ethyl acetate 4:1) to afford Compound 275A as a white solid (500 mg, 57%). MS-ESI: [M+H]⁺=537.3. ¹H NMR (400 MHz, CDCl₃): δ 7.59-7.44 (m, 6H), 7.28-7.25 (m, 2H), 4.61 (m, 1H), 3.50 (m, 1H), 3.14 (m, 2H), 2.68-2.64 (m, 2H), 1.75-1.62 (m, 4H), 1.49-1.39 (m, 15H), 0.96 (t, J=7.4 Hz, 3H).

Compound 275A was separated by SFC (Column: Chiralcel OJ-H 250×4.6 mm I.D., Sum. Mobile phase: methanol (0.05% DEA) in CO₂ from 5% to 40%. Flow rate: 2.35 mL/min. Wavelength: 220 nm) to give peak 1 (compound 275 P1, 90 mg) and peak 2 (compound 275 P2, 300 mg).

Compounds 275 and 276 were prepared using General Methods 1 and 13 from Compound 275A P1. Both 275 and 276 are obtained upon prep-HPLC purification (C18, CH₃CN/H₂O plus 0.05% HCl).

Analytical data for Compound 275: MS (ESI) m/z 949.2 (M+Na)⁺; t_{R} 2.73 min (10% CH₃CN/H₂O−80% CH₃CN/H₂O, 4 min, 0.8 mL/min, Xtimate C18, 2.1×30 mm).

Analytical data for Compound 276: MS (ESI) m/z 379.2 ((M-36)/2+H)⁺; t_{R} 2.76 min (10% CH₃CN/H₂O−80% CH₃CN/H₂O, 4 min, 0.8 mL/min, Xtimate C18, 2.1×30 mm).

### Example 106

### Preparation of Compounds 277 and 278 (derived from Compound 275 P2)

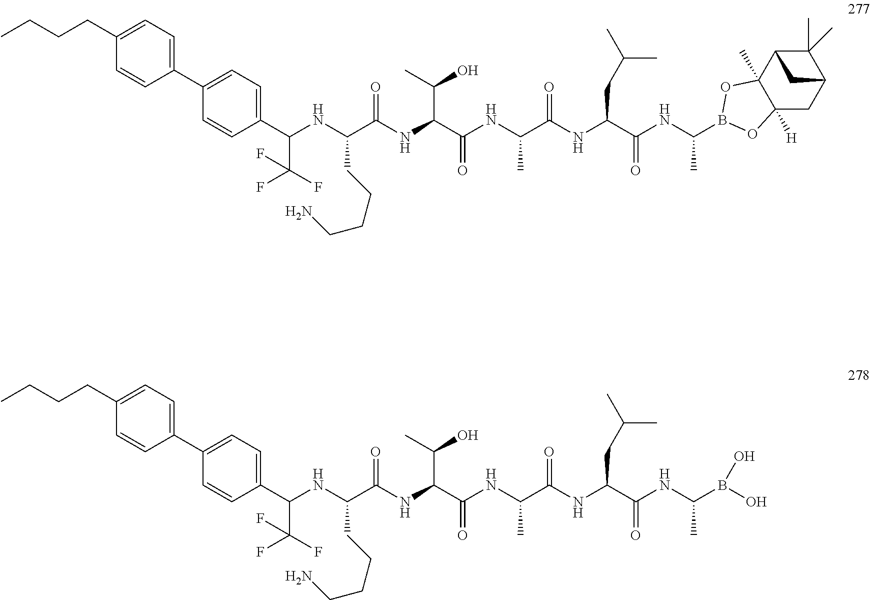

Compounds 277 and 278 were prepared using General Methods 1 and 13 from Compound 275A P2. Both compounds 277 and 278 are obtained upon prep-HPLC purification (C18, CH₃CN/H₂O plus 0.05% HCl).

Analytical data for Compound 277: MS (ESI) m/z 464.4 (M/2+H)⁺; t_{R} 2.75 min (10% CH₃CN/H₂O−80% CH₃CN/H₂O, 4 min, 0.8 mL/min, Xtimate C18, 2.1×30 mm).

Analytical data for Compound 278: MS (ESI) m/z 379.2 ((M-36)/2+H)⁺; t_{R} 2.76 min (10% CH₃CN/H₂O−80% CH₃CN/H₂O, 4 min, 0.8 mL/min, Xtimate C18, 2.1×30 mm).

### Example 107

### Preparation of Compound 279

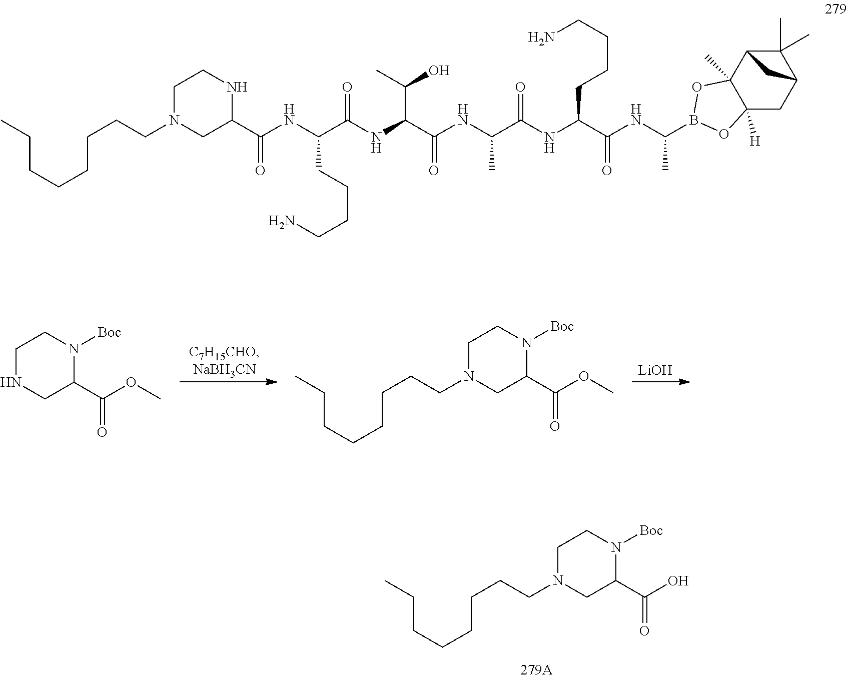

To a mixture of octanal (2 g, 17.8 mmol) in DCM (50 mL) was added 1-tert-butyl 2-methyl piperazine-1,2-dicarboxylate (4.8 g, 19.6 mmol), HOAc (1 mL) and NaBH₃CN (1.66 g, 26.8 mmol) at 0° C. The mixture was stirred at 30° C. for 12 h. The reaction mixture was added H₂O (50 mL), in which the aqueous layer was extracted by DCM (50 mL×2). The combined organic layers were dried over Na₂SO₄ and concentrated; and the residue was purified by silica gel column (PE/EA=20:1) to give 1-tert-butyl 2-methyl 4-octylpiperazine-1,2-dicarboxylate as a light yellow oil (4.0 g, 63%).

The ester hydrolysis of 1-tert-butyl 2-methyl 4-octylpiperazine-1,2-dicarboxylate (4.0 g, 11.2 mmol) was performed with LiOH in a manner similar to that described for compound 215C to afford compound 279A as a yellow oil (3.7 g, 97%).

Compound 279 was prepared as the HCl salt using General Methods 1 and 13 from Compound 279A and compound 126D2. LC-MS (General Method 12): MS (ESI) m/z 898.8 (M+Na)⁺; t_{R} 0.753 min.

### Example 108

### Preparation of Compound 280

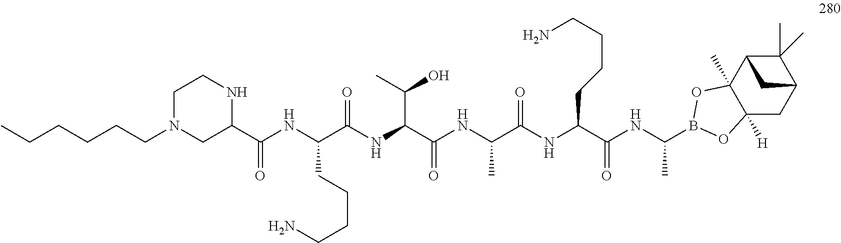 [SMILES: CCCCCCN1CCNC(C(=O)N[C@@H](CCCCN)C(=O)N[C@H](C(=O)N[C@@H](C)C(=O)N[C@@H](CCCCN)C(=O)N[C@@H](C)B2O[C@@H]3C[C@@H]4C[C@@H](C4(C)C)[C@]3(C)O2)[C@@H](C)O)C1]

Compound 280 was prepared as the HCl salt using General Methods 1 and 13 in a manner similar to compound 279 except hexanal was used as the starting material. LC-MS (General Method 12): MS (ESI) m/z 848.4 (M+H)⁺; t_{R} 0.724 min.

### Example 109

### Preparation of Compound 281

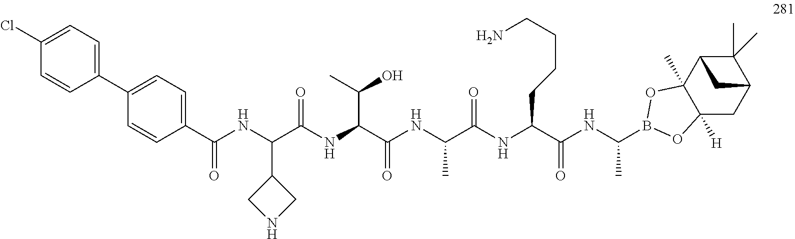 [SMILES: C[C@H](NC(=O)[C@H](CCCCN)NC(=O)[C@H](C)NC(=O)[C@@H](NC(=O)C(NC(=O)c1ccc(-c2ccc(Cl)cc2)cc1)C1CNC1)[C@@H](C)O)B1O[C@@H]2C[C@@H]3C[C@@H](C3(C)C)[C@]2(C)O1]

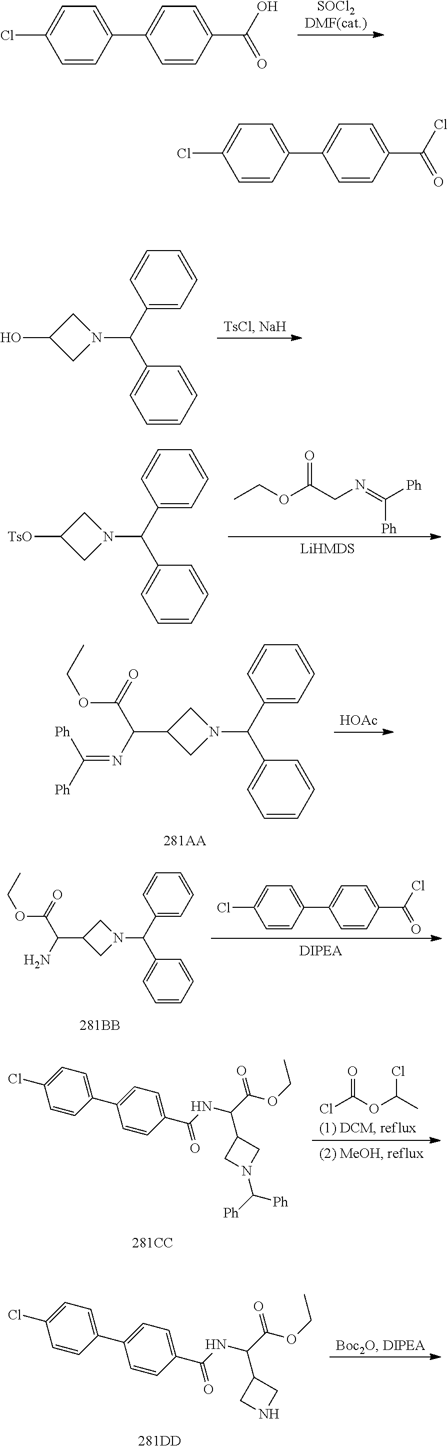

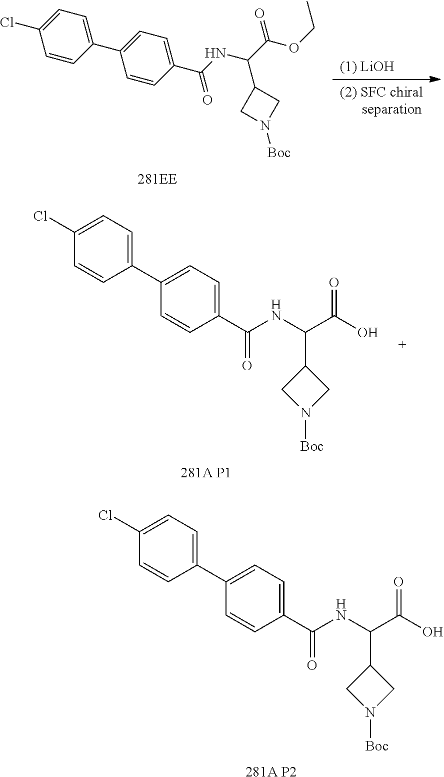

To a solution of 4′-chloro-[1,1′-biphenyl]-4-carboxylic acid (8.7 g, 37.2 mmol) in DCM (200 mL) was added SOCl₂ (22 g, 186 mmol) and DMF (0.5 ml) at room temperature. The mixture was refluxed for 4 h. After TLC showed that the reaction was completed, reaction volatiles were removed under reduced pressure to obtain 4′-chloro-[1,1′-biphenyl]-4-carbonyl chloride as a yellow solid (9.25 g, 98%).

To a solution of 1-benzhydrylazetidin-3-ol (50.7 g, 212 mmol) in anhydrous THF (700 mL) was added NaH (38.2 g, 954 mmol) at 0° C. After stirring for 30 min, p-TsCl (80.6 g, 424 mmol) was added to the mixture in portions at 0° C. The resultant mixture was allowed to warm to 20° C. and stirred for 18 h. After TLC showed the reaction was completed, the mixture was poured into crushed ice, where the aqueous layer was further extracted with DCM (300 mL×2). The combined organic layers were dried over Na₂SO₄ and concentrated under reduced pressure, and the residue was purified on silica gel column (PE/EtOAc=10:1 to 6:1) to give 1-benzhydrylazetidin-3-yl 4-methylbenzenesulfonate as a white solid (55 g, 77%).

To a solution of 1-benzhydrylazetidin-3-yl 4-methylbenzenesulfonate (55 g, 162 mmol) and ethyl 2-((diphenylmethylene)amino)acetate (44 g, 164 mmol) in anhydrous toluene (400 mL) was added LiHMDS (198 mL, 198 mmol) dropwise at 0° C. After that, the mixture was heated at 110° C. for 2 h. After TLC showed that the reaction was completed, the mixture was quenched with water (400 mL), where the aqueous layer was further extracted with EtOAc (300 mL×3). The combined organic layers were dried over anhydrous Na₂SO₄ and concentrated under reduced pressure to get the residue, which was purified on silica gel column (PE/EA=15:1˜10:1) to give pure compound 281AA as brown oil (38 g, 48%).

To a suspension of compound 281AA (38 g, 84.7 mmol) in THF (65 mL) and water (65 mL) was added acetic acid (65 mL) at 0° C., and the mixture was stirred at 20° C. for 18 h. After the TLC showed the reaction was completed, the mixture was diluted with water (700 mL), and brought to pH 8˜9 by addition of solid Na₂CO₃ (ca. 230 g), where the aqueous phase was extracted with EtOAc (400 mL×3). The combined organic layers were washed with brine (600 mL), dried over anhydrous MgSO₄ and concentrated under reduced pressure; and the residue was purified by silica gel column (PE/EA=3:1˜1:1) to afford compound 281BB as a pale yellow oil (18.5 g, 67%).

To a solution of compound 281BB (12 g, 37 mmol) and DIPEA (9.56 g, 74 mmol) in DCM (170 mL) was added 4′-chloro-[1,1′-biphenyl]-4-carbonyl chloride (9.25 g, 37 mmol) in DCM (20 mL) dropwise at 0° C. The reaction mixture was stirred at 20° C. for 6 h. After TLC showed that the reaction was completed, the mixture was washed with water (200 mL), where the aqueous layer was further extracted by DCM. The combined organic layers were collected, dried over NaSO₄ and concentrated to give the residue, which was purified by silica gel column (PE/EA=5:1) to give compound 281CC as a white solid (15 g, 75.3%).

To a solution of compound 281CC (13 g, 24 mmol) in DCM (60 mL) was added 1-chloroethyl chloroformate (6.8 g, 48 mmol) in DCM (30 mL) dropwise at 0° C. under N₂. The reaction was stirred for 30 min and then heated to reflux for 12 h. After TLC showed the disappearance of starting material, MeOH (100 mL) was added and the mixture was heated to reflux for another 1 h. The mixture was concentrated under pressure to give compound 281DD (8.8 g, 98%), which was used directly.

To a solution of compound 281DD (8.9 g, 23.9 mmol) and DIPEA (4.6 g, 35.9 mmol) in DCM (15 mL) was added Boc₂O (5.3 g, 23.9 mmol) at 0° C. The reactions mixture was stirred at 20° C. for 8 h. After TLC showed that the reaction was completed, the mixture was concentrated under reduced pressure to give the residue, which was purified by silica gel column (PE/EA=5:1 to 4:1) to give compound 281EE (4.5 g, 41%).

Typical ester hydrolysis under LiOH was applied to compound 281EE (4.5 g, 9.53 mmol) to obtain the carboxylic acid product as a white solid (4.0 g), which was further separated by chiral SFC to afford compound 281 P1 (1.5 g, 36%) and compound 281 P2 (1.7 g, 40%) as their individual enantiomers.

Compound 281 was prepared as the HCl salt using General Methods 1 and 13 from Compound 281A P1. LC-MS (General Method 12): MS (ESI) m/z 850.1 (M+Na)⁺; t_{R} 0.786 min.

### Example 110

### Preparation of Compound 282

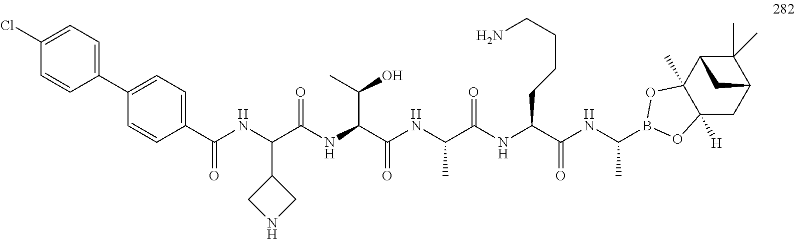 [SMILES: C[C@H](NC(=O)[C@H](CCCCN)NC(=O)[C@H](C)NC(=O)[C@@H](NC(=O)C(NC(=O)c1ccc(-c2ccc(Cl)cc2)cc1)C1CNC1)[C@@H](C)O)B1O[C@@H]2C[C@@H]3C[C@@H](C3(C)C)[C@]2(C)O1]

Compound 282 was prepared as the HCl salt using General Methods 1 and 13 from Compound 281A P2. LC-MS (General Method 12): MS (ESI) m/z 850.1 (M+Na)⁺; t_{R} 0.788 min.

### Biological Data

### Example 111

### Determination of Minimum Inhibitory Concentration

In vitro antimicrobial activity of each compound was determined by measuring minimal inhibitor concentrations (MICs) using the broth micro-dilution technique as approved by the Clinical and Laboratory Standards Institute (CLSI). Antibacterial activity was measure against two strains of bacteria: 1) methicillin resistant Staphylococcus aureus (MRSA) strain USA300 (NRS384) and 2) Escherichia coli strain MC4100 IMP-4213, which harbors an LptD mutation. Bacterial inocula were prepared by scraping cells into 1 mL of testing media (cation adjusted Mueller Hinton Broth supplemented with 0.002% v/v Tween-80) and diluting to a final OD₆₀₀ₙₘ of 0.01.

Test compounds were prepared in DMSO at a concentration of 10 mg/ml. These compound stocks were diluted into testing media at a concentration of 64 μg/ml and 9 serial 1:2 dilutions were made in the same media, in 96-well U bottom microtiter dishes. Bacterial inocula were added to the two fold serial dilutions of test compounds to a final density of OD OD₆₀₀ₙₘ of 0.0005 and incubated stationarily at 35° C. for 22 hours, after which the plates were examined visually. The MICs were recorded as the lowest concentration of test compound that completely prevented bacterial growth. The results are listed in Table 1.

TABLE 1
Antimicrobial activities in whole cell bacterial assays

| | MIC (μg/mL) | MIC (μg/mL) |
|---|---|---|
| Compound | E. coli | S. aureus |
| 101 | >64 | >64 |
| 102 | >64 | 32 |
| 103 | >64 | >64 |
| 104 | >64 | 64 |
| 105 | 64 | 51 |
| 106 | >64 | >64 |
| 107 | >64 | >64 |
| 108 | >64 | >64 |
| 109 | 32 | 32 |
| 110 | 64 | 32 |
| 111 | 16 | 16 |
| 112 | 64 | 1 |
| 113 | >64 | >64 |
| 114 | 32 | 13 |
| 115 | >64 | 11 |
| 116 | 32 | 1.4 |
| 117 | 8 | 2 |
| 118 | 8 | 2 |
| 119 | 2 | 1 |
| 120 | 11 | 2.8 |
| 121 | 1.3 | 0.4 |
| 122 | 0.2 | 0.4 |
| 123 | 4 | 2 |
| 124 | 0.6 | 0.8 |
| 125 | 0.4 | 0.4 |
| 126 | >64 | 64 |
| 127 | 32 | 2 |
| 128 | 64 | 2 |
| 129 | 4 | 2.8 |
| 130 | >64 | 64 |
| 131 | nt | 1 |
| 132 | 32 | 5.7 |
| 133 | >64 | 8 |
| 201 P1 | 1 | 0.09 |
| 201 P2 | 5.7 | 2 |
| 203 P1 | 11 | 1.2 |
| 203 P2 | 23 | 8 |
| 205 P1 | 32 | >64 |
| 205 P2 | 32 | >64 |
| 207 P1 | 8 | 5.7 |
| 207 P2 | 32 | 32 |
| 209 | 8 | 1.6 |
| 210 | 4 | 11 |
| 211 | 16 | 4 |
| 212 | 64 | 45 |
| 213 | 64 | 32 |
| 214 | 32 | 64 |
| 215 | 64 | 6.7 |
| 216 | 16 | 1 |
| 217 | 23 | 0.31 |
| 218 | >64 | 2.1 |
| 219 | 16 | 1 |
| 220 | 16 | 1 |
| 221 | 64 | 4 |
| 222 | 8 | 2 |
| 223 | 64 | 4 |
| 224 | >64 | 11 |
| 225 | 16 | 2 |
| 226 | >64 | 6.3 |
| 227 | 64 | 2 |
| 228 | >64 | 2 |
| 229 | 64 | 1.3 |
| 230 | 32 | 0.36 |
| 231 | >64 | 32 |
| 232 | 11 | 1.5 |
| 233 | 8 | 2 |
| 234 | 32 | 16 |
| 235 | 32 | 16 |
| 236 | 16 | 0.5 |
| 237 | 32 | 0.35 |
| 238 | 32 | 0.18 |
| 239 | 4 | 0.25 |
| 240 | 32 | 1.4 |
| 241 | 32 | 1 |
| 242 | 64 | 4 |
| 243 | >64 | 1.4 |
| 244 | 64 | 0.87 |
| 245 | 32 | 0.35 |
| 246 | >64 | 2.3 |
| 247 | >64 | 3.2 |
| 248 | >64 | 1 |
| 249 | >64 | 0.71 |
| 250 | >64 | 0.5 |
| 251 | >64 | 0.5 |
| 252 | 64 | 1 |
| 253 | 64 | 1 |
| 254 | nt | 16 |
| 255 | >64 | 11 |
| 256 | >64 | 10 |
| 257 | >64 | 11 |
| 258 | >64 | 8 |
| 259 | 64 | 2.4 |
| 260 | 32 | 23 |
| 261 | 4 | 1.7 |
| 262 | 4 | 5 |
| 263 | 0.25 | 0.71 |
| 264 | 4 | 8 |
| 265 | 16 | 16 |
| 266 | 16 | 23 |
| 267 | 32 | 11 |
| 268 | 8 | 0.5 |
| 269 | 11 | 0.35 |
| 270 | 0.25 | 0.25 |
| 271 | 0.13 | 0.25 |
| 272 | 0.13 | 0.13 |
| 273 | 0.13 | 0.13 |
| 274 | 0.5 | 0.13 |
| 275 | nt | nt |
| 276 | nt | nt |
| 277 | 4 | 1.4 |
| 278 | nt | nt |
| 279 | >64 | 10 |
| 280 | >64 | 64 |
| 281 | 8 | 4 |
| 282 | 16 | 8 |

nt = not tested

### Example 112

### Enzyme Inhibition Assay

Full length His-tagged E. coli SPase proteins were expressed in E. coli BL21(DE3) containing the plasmid pET23-lepB. Briefly, saturated overnight cultures grown in 20 ml of Luria-Bertani medium supplemented with ampicillin were subcultures into 1.5 L of Luria-Bertani, and shaken at 37° C. until an optical density at 600 nm of 0.4-0.5 was achieved. Protein expression was induced with Isopropyl β-D-1-thiogalactopyranoside (ITPG) at a final concentration of 0.5 μM, and purified using nickel affinity chromatography.

Full length His-tagged S. aureus SPase protein was expressed similarly from E. coli BL21(DE3) containing the plasmid pCDF1-SaSpsB and purified similarly to the E. coli protein with the following exceptions. SPase protein was solubilized using 300 mM NaCl, 20 mM Tris pH 8.06, 5 mM Imidazole, 10% glycerol, 1% Triton X-100, prior to purification in Ni-NTA Superflow resin and resin bound protein was washed in a similar buffer containing 1% Elugent in place of Triton X-100 prior to protein eluted in wash buffer supplemented with 300 mM imidazole. Protein purity was judged to exceed 95% by visual inspection of SDS-PAGE followed by Comassie staining. All protein concentrations were determined by BCA assay.

Signal peptidase enzyme activity of the above proteins was measured using two fluorogenic peptide substrates (decanoyl-LSSPAYNO2A [symbol] ADKabzPD and decanoyl-LTPTAYNO2A [symbol] ASKKabzDD), where abz is the fluorescence donor 2-aminobenzamide, YNO2 is the fluorescence acceptor 3-nitrotyrosine, and the cleavage site is indicated with an arrow. Enzyme mix solution was prepared by diluting 2.5 nM of Escherichia coli or Staphylococcus aureus SPase protein into reaction buffers consisting of 20 mM PO4 pH 7.4, 100 mM NaCl, and 1% Elugent™ or octyl phenoxypolyethoxylethanol detergent at a concentration of 0.25% or 0.0625%. Reactions were initiated by the addition of substrate to a final concentration of 20 μM. Reaction progress was monitored by measuring the increase in fluorescence signal (excitation at 314 nm, emission at 416 nm) using a SpectraMax M2 fluorescence microplate reader. To determine IC50 values of test compounds, compound stock solutions were prepared in DMSO at a concentration of 1 mM. Three-folder serial dilutions of test compounds, starting at 10 μM, were prepared in enzyme mix solution and incubated at room temperature for 10 minutes. Following this incubation, fluorogenic substrate was added to a final concentration of 20 μM and the increase in fluorescence, corresponding to substrate cleavage, was monitored continuously at room temperature for 1 hour. Initial reaction rates were calculated based on the rate of increase in fluorescence during the reaction. Reaction rates were plotted as a function of compound concentration, and IC₅₀ values are determined nonlinear regression analysis (SoftMaxPro 5.4, Molecular Devices™) of the sigmoidal dose-response curve. The results are listed in Table 2.

TABLE 2
Inhibitory activities (IC50) in biochemical SPase activity assays

| | IC 50 (nM) | IC 50 (nM) |
|---|---|---|
| Compound | E. coli | S. aureus |
| 101 | 870 | 1600 |
| 102 | 440 | 580 |
| 103 | 1100 | 1400 |
| 104 | 950 | 840 |
| 105 | 900 | 1300 |
| 106 | 6200 | 45 |
| 107 | 3200 | 360 |
| 108 | 930 | 3700 |
| 109 | 820 | 2000 |
| 110 | 6000 | 3800 |
| 111 | 360 | 690 |
| 112 | nt | nt |
| 113 | 5700 | 1200 |
| 114 | 64 | 250 |
| 115 | 330 | 590 |
| 116 | 200 | 480 |
| 117 | 20 | 150 |
| 118 | 15 | 70 |
| 119 | 10 | 120 |
| 120 | 21 | 120 |
| 121 | 54 | 280 |
| 122 | 20 | 240 |
| 123 | 11000 | 7800 |
| 124 | 110 | 2000 |
| 125 | 10 | 280 |
| 126 | 230 | 250 |
| 127 | 64 | 210 |
| 128 | 1200 | 75 |
| 129 | 3.6 | 120 |
| 130 | 270 | 280 |
| 131 | nt | nt |
| 133 | 7000 | 8800 |
| 134 | 3800 | 120 |
| 201 P1 | 7 | 10 |
| 201 P2 | 150 | 210 |
| 203 P1 | 440 | 95 |
| 203 P2 | 1500 | 1000 |
| 205 P1 | >10000 | >10000 |
| 205 P2 | >10000 | >10000 |
| 207 P1 | 1800 | 1400 |
| 207 P2 | 9900 | 1300 |
| 209 | 16 | 85 |
| 210 | 260 | 2300 |
| 211 | 380 | 390 |
| 212 | 34 | 2600 |
| 213 | 49 | >10000 |
| 214 | 180 | 630 |

nt = not tested

### Example 113

### Checkerboard Synergy Assays

2D MIC assay or “checkerboard assays” are the most common method used to quantify synergistic or antagonistic interactions between two antibiotics with respect to potency (Hallander, H. O., et al., Antimicrob. Agents Chemother. 1982 22:743-752). In this assay in each axis of a 96-well plate contains a 2-fold dilution of a given agent, such that each well contains a unique combination of the agents being tested.

To create a checkerboard dilution scheme, imipenem was diluted in Mueller Hinton II Broth+0.002% Tween-80 to twice the final desired concentration, and six 2-fold serial dilutions were performed in the same media yielding seven imipenem concentrations. Dilutions of Compound 217 were prepared similarly except that ten dilutions were performed for a total of eleven concentrations. For each concentration of imipenem, 50 uL aliquots were transferred to columns 1-12 of a given row of a 96-well clear polypropylene assay plate. For each concentration of Compound 217, 50 uL were transferred to rows A-H of a given column of the same plate. The resulting plate contained imipenem serially diluted on the Y-axis and Compound 217 serially diluted along the X-axis.

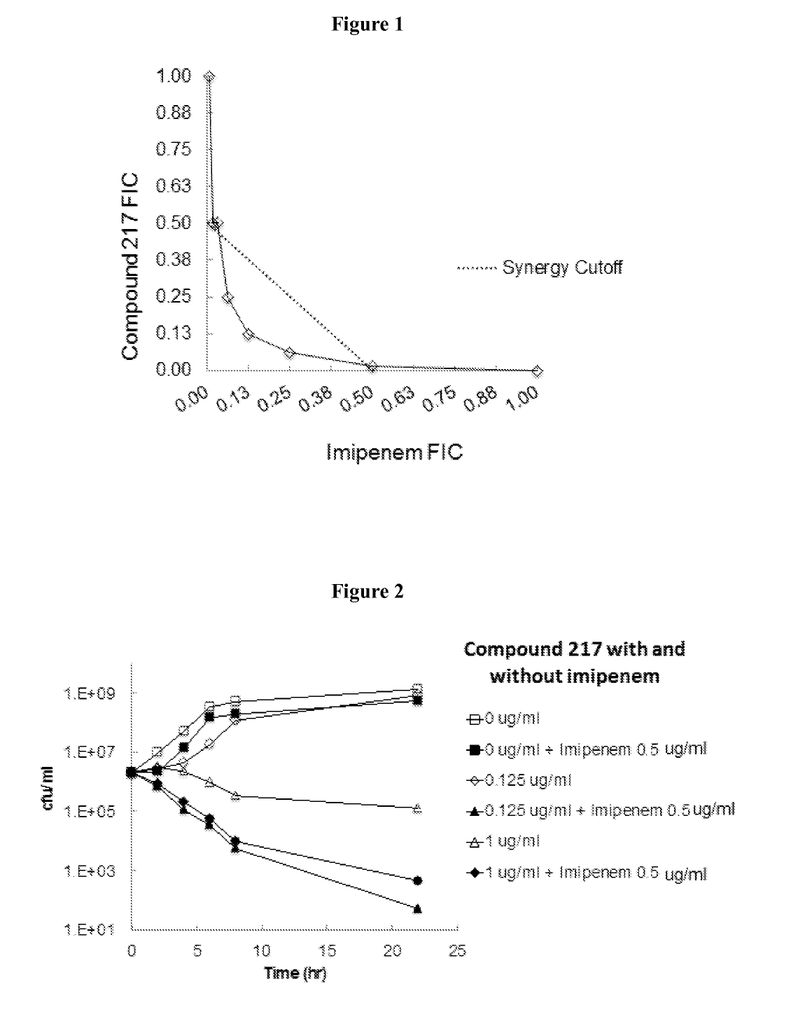

MRSA strain USA300 was grown overnight at 35° C. on Mueller Hinton Agar plates, and colonies were suspended in Mueller Hinton II Broth+0.002% Tween-80 to a final density of 1*10⁷ cfu/ml. To each well of the above plate, 5 ul of this suspension were added, resulting in an initial density of 5*10⁵ cfu/ml. The plate was incubated at 35° C. for 22 hours after which growth was determined via visual inspection. For each sub-MIC concentration of imipenem, the concentration of Compound 217 required to prevent visible growth was recorded, and the fractional inhibitor concentration (FIC) of each agent was calculated by dividing the concentration of each agent by MIC of the agent alone. FICs are plotted in FIG. 1 generating an isobologram, and synergy is defined as any point where the sum of the FICs is ≦0.5. Examination of FIG. 1 reveals significant synergy between Compound 217 and imipenem as evidenced by many points where the sum of the FICs is <0.5.

### Example 114

### Time-Kill Assays

Time-kill experiments enable quantification of rate of bacterial growth or death in the presence of a fixed concentration of one or more antibiotics (Arhin. F., et. al., Current Protocols in Microbiology 17.1.1-17.1.22, February 2010). Time-kill experiments were performed in 96-well incubation plates using Mueller Hinton II Broth+0.002% Tween-80 as the growth media. Individual wells contained imipenem alone, Compound 217 alone, or a combination of both agents at various concentrations. MRSA strain USA300 was grown overnight at 35° C. on Mueller Hinton Agar plates, and colonies were suspended in Mueller Hinton II Broth+0.002% Tween-80 to a final density of 2*10⁸ cfu/mL. To each well of the incubation plates, 5 uL of this suspension were added, resulting in an initial density of 1*10⁷ cfu/mL. Plates were incubated at 35° C., and at various time points 30 μl samples were removed, mixed 1:1 with 25 mg/ml activated carbon, serially diluted in sterile phosphate buffered saline supplemented with 0.05% Tween 20, and spotted onto Mueller Hinton Agar plates to enable cfu quantification. Colonies were counted and the cfu/ml at each time point calculated by based on the dilution factor and volume spotted. FIG. 2 shows the results from a representative time-kill assay. As seen from these results, the combination of 0.5 ug/mL imipenem plus 0.125 ug/mL Compound 217 is synergistic by time-kill assay, resulting in a faster and more extensive reduction of viable cells than either agent alone.

### Example 115

### Synergy of SPase Inhibitors with Partner β-Lactam Antibiotics

Many compounds in the Examples, including Compound 217, synergize with a wide range of beta-lactam antibiotics as quantified by SIC determination, where the SIC is measured and defined in a manner identical to the MIC, with the exception that the testing media contains a partner beta-lactam at a concentration equal to ¼× its MIC against the bacterial strain being tested. When the SIC of a compound is less than or equal to ¼ its MIC, the sum of the FICs for the compound and the partner beta-lactams is ≦0.5 indicating synergy (Hallander, H. O., et al., Antimicrob. Agents Chemother. 1982 22:743-752).

TABLE 3

MRSA strain USA300

| | Compound 217 | Fold reduction in |
|---|---|---|
| | MIC | Compound 217 |
| Partner beta-lactam | (μg/ml) | MIC |
| None | 0.5 | NA |
| | Compound 217 | Fold reduction in |
| | SIC | Compound 217 |
| Partner beta-lactam | (μg/ml) | MIC |
| Azlocillin | 0.125 | 4 |
| Amoxicillin/Clav | 0.125 | 4 |
| Ampicillin | 0.25 | 2 |
| Doripenem | 0.03125 | 16 |
| Meropenem | 0.01563 | 32 |
| Biapenem | 0.0156 | 32 |
| Cefamandole | 0.125 | 4 |
| Imipenem | 0.03125 | 16 |
| Mezlocillin | 0.125 | 4 |
| Cefmetazole | 0.125 | 4 |
| Cefprozil | 0.125 | 4 |
| Piperacillin/tazobactam | 0.125 | 4 |
| Carbenicillin | 0.125 | 4 |
| Cefaclor | 0.125 | 4 |
| Cephalothin | 0.03125 | 16 |
| Ertapenem | 0.125 | 4 |
| Cefazolin | 0.0625 | 8 |
| Cefepime | 0.03125 | 16 |
| Cefonicid | 0.125 | 4 |
| Cefoxitin | 0.125 | 4 |
| Ceftazidime | 0.125 | 4 |
| Oxacillin | 0.0625 | 8 |
| Cefdinir | 0.0078 | 64 |
| Cefixime | 0.0625 | 8 |
| Cefotaxime | 0.0625 | 8 |
| Cefotetan | 0.125 | 4 |
| Cefpodoxime | 0.0625 | 8 |
| Ceftizoxime | 0.0625 | 8 |
| Ceftriaxone | 0.03125 | 16 |
| Faropenem | ≦0.0039 | >128 |
| Mecillinam | 0.25 | 2 |
| Methicillin | 0.125 | 4 |
| Moxalactam | 0.03125 | 16 |
| Ticarcillin | 0.0625 | 8 |

The data in Table 3 demonstrate that Example compounds synergize with Azlocillin, Amoxicillin, Ampicillin, Doripenem, Meropenem, Biapenem, Cefamandole, Imipenem, Mezlocillin, Cefmetazole, Cefprozil, Piperacillin/tazobactam, Carbenicillin, Cefaclor, Cephalothin, Ertapenem, Cefazolin, Cefepime, Cefonicid, Cefoxitin, Ceftazidime, Oxacillin, Cefdinir, Cefixime, Cefotaxime, Cefotetan, Cefpodoxime, Ceftizoxime, Ceftriaxone, Faropenem, Mecillinam, Methicillin, Moxalactam, and Ticarcillin.

### Example 116

### SpsB Inhibitor/Imipenem Intraperitoneal Delivery in Neutropenic Thigh Infection Model

CD-1 mice are induced neutropenia (100 cells/mm³) by injecting 150 mg/kg and 100 mg/kg cyclophosphamide at day −5 and day −2 respectively. At day 0, mice are infected in the thigh muscle with MRSA strain COL 4×10⁵ CFU/50 μL. At 2 hours post infection, the SpsB inhibitor at 40 mg/kg is delivered intraperitoneally. At the same time, 10 mg/kg of imipenem/cilastatin is administered subcutaneously into the same mouse. At 8, 12, or 24 hours post infection, bacterial burden in the thigh muscle is determined by plating the tissue homogenate in series dilutions on blood agar plates. Other beta-lactam antibiotics may be used in place of imipenem in this model.

### Example 117

### Clinical Trial of the Safety and Efficacy of Compounds of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) in Patients with C. Difficile-Associated Diarrhea

Purpose:

This study aims to determine the safety and efficacy of compounds presented herein for the treatment of symptoms of C. difficile-associated diarrhea and lowering the risk of repeat episodes of diarrhea. The compounds are evaluated in comparison to current standard antibiotic treatment, so all patients will receive active medication. All study-related care is provided including doctor visits, physical exams, laboratory tests and study medication. Total length of participation is approximately 10 weeks.

Patients:

Eligible subjects will be men and women 18 years and older.

Criteria:

Inclusion Criteria:

Be at least 18 years old;

Have active mild to moderate C. difficile-Associated Diarrhea (CDAD);

Be able to tolerate oral medication;

Not be pregnant or breast-feeding; and

Sign and date an informed consent form.

Study Design:

This is a randomized, double-blind, active control study of the efficacy, safety, and tolerability of a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) in patients with C. difficile-associated diarrhea.

### Example 118

### Clinical Trial Comparing a Compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) with Vancomycin for the Treatment of MRSA Osteomyleitis

Purpose:

This study aims to determine the efficacy of compounds presented herein as compared to vancomycin for the treatment of methicillin-resistant Staphylococcus aureus (MRSA) osteomyelitis.

Patients:

Eligible subjects will be men and women 18 years and older.

Criteria:

Inclusion Criteria:
Culture-proven MRSA, obtained in operating room or sterile biopsy procedure from bone site. The infection and sampling site is either within the bone or a deep soft-tissue site that is contiguous with bone; OR radiographic abnormality consistent with osteomyelitis in conjunction with a positive blood culture for MRSA;
Surgical debridement of infection site, as needed;
Subject is capable of providing written informed consent; and
Subject capable of receiving outpatient parenteral therapy for 12 weeks.

Exclusion Criteria:
Hypersensitivity to a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or vancomycin;
S. aureus resistant to a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) or vancomycin;
Osteomyelitis that develops directly from a chronic, open wound;
Polymicrobial culture (the only exception is if coagulase-negative staphylococcus is present in the culture and the clinical assessment is that it is a contaminant);
Subject has a positive pregnancy test at study enrollment;
Baseline renal or hepatic insufficiency that would preclude administration of study drugs;
Active injection drug use without safe conditions to administer intravenous antibiotics for 3 months; and
Anticipated use of antibiotics for greater than 14 days for an infection other than osteomyelitis.

Study Design:

This is a randomized, open-label, active control, efficacy trial comparing vancomycin with a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) for the treatment of MRSA Osteomyelitis.

### Example 119

### Clinical Trial Evaluating a Compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) in Selected Serious Infections Caused by Vancomycin-Resistant Enterococcus (VRE)

Purpose:

This study aims to determine the safety and efficacy of a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) in the treatment of selected serious infections caused by VRE.

Patients:

Eligible subjects will be men and women 18 years and older.

Criteria:

Inclusion Criteria:
Isolation of one of the following multi-antibiotic resistant bacteria: vancomycin-resistant Enterococcus faecium, vancomycin-resistant Enterococcus faecalis alone or as part of a polymicrobial infection; and
Have a confirmed diagnosis of a serious infection (eg, bacteremia [unless due to an excluded infection], complicated intra-abdominal infection, complicated skin and skin structure infection, or pneumonia) requiring administration of intravenous (IV) antibiotic therapy.

Exclusion Criteria:
Subjects with any concomitant condition or taking any concomitant medication that, in the opinion of the investigator, could preclude an evaluation of a response or make it unlikely that the contemplated course of therapy or follow-up assessment will be completed or that will substantially increase the risk associated with the subject's participation in this study.
Anticipated length of antibiotic therapy less than 7 days

Study Design:

This is a randomized, double-blind, safety and efficacy study of a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) in the treatment of selected serious infections caused by VRE.

### Pharmaceutical Compositions

### Parenteral Composition

To prepare a parenteral pharmaceutical composition suitable for administration by injection, 100 mg of a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) is dissolved in DMSO and then mixed with 10 mL of 0.9% sterile saline. The mixture is incorporated into a dosage unit form suitable for administration by injection.

In another embodiment, the following ingredients are mixed to form an injectable formulation:

| Ingredient | Amount | |
|---|---|---|
| Compound of Formula (A), (A′), (I), (I′), (II), (II′), | 1.2 | g |
| (III), (III′), (IV), (IV′), (V), (V′), (VI), (VI′), (VII), | | |
| (VII′), (VIII), (VIII′), (IX), (IX′), (X), (X′), (XI), | | |
| (XI′), (XII), (XII′), (XIII), (XIII′), (XIV), (XIV′), | | |
| (XV), or (XV′) | | |
| sodium acetate buffer solution (0.4M) | 2.0 | mL |

| | |
|---|---|
| HCl (1N) or NaOH (1M) | q.s. to suitable pH |
| water (distilled, sterile) | q.s. to 20 mL |

All of the above ingredients, except water, are combined and stirred and if necessary, with slight heating if necessary. A sufficient quantity of water is then added.

### Oral Composition

To prepare a pharmaceutical composition for oral delivery, 100 mg of a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) is mixed with 750 mg of starch. The mixture is incorporated into an oral dosage unit, such as a hard gelatin capsule, which is suitable for oral administration.

In another embodiment, the following ingredients are mixed intimately and pressed into single scored tablets.

| | Quantity per |
|---|---|
| Ingredient | tablet, mg |
| compound of Formula (A), (A′), (I), (I′), (II), (II′), | 200 |
| (III), (III′), (IV), (IV′), (V), (V′), (VI), (VI′), (VII), | |
| (VII′), (VIII), (VIII′), (IX), (IX′), (X), (X′), (XI), | |
| (XI′), (XII), (XII′), (XIII), (XIII′), (XIV), (XIV′), | |
| (XV), or (XV′) | |
| Cornstarch | 50 |
| croscarmellose sodium | 25 |
| Lactose | 120 |
| magnesium stearate | 5 |

In yet another embodiment, the following ingredients are mixed intimately and loaded into a hard-shell gelatin capsule.

| | Quantity per |
|---|---|
| Ingredient | tablet, mg |
| compound of Formula (A), (A′), (I), (I′), (II), (II′), | 200 |
| (III), (III′), (IV), (IV′), (V), (V′), (VI), (VI′), (VII), | |
| (VII′), (VIII), (VIII′), (IX), (IX′), (X), (X′), (XI), | |
| (XI′), (XII), (XII′), (XIII), (XIII′), (XIV), (XIV′), | |
| (XV), or (XV′) | |
| lactose, spray-dried | 148 |
| magnesium stearate | 2 |

In yet another embodiment, the following ingredients are mixed to form a solution/suspension for oral administration:

| Ingredient | Amount | |
|---|---|---|
| Compound of Formula (A), (A′), (I), (I′), (II), (II′), | 1 | g |
| (III), (III′), (IV), (IV′), (V), (V′), (VI), (VI′), (VII), | 0.1 | g |
| (VII′), (VIII), (VIII′), (IX), (IX′), (X), (X′), (XI), | | |
| (XI′), (XII), (XII′), (XIII), (XIII′), (XIV), (XIV′), | | |
| (XV), or (XV′) | | |
| Anhydrous Sodium Carbonate | | |
| Ethanol (200 proof), USP | 10 | mL |
| Purified Water, USP | 90 | mL |
| Aspartame | 0.003 | g |

### Topical Gel Composition

To prepare a pharmaceutical topical gel composition, 100 mg of a compound of Formula (A), (A′), (I), (I′), (II), (II′), (III), (III′), (IV), (IV′), (V), (V′), (VI), (VI′), (VII), (VII′), (VIII), (VIII′), (IX), (IX′), (X), (X′), (XI), (XI′), (XII), (XII′), (XIII), (XIII′), (XIV), (XIV′), (XV), or (XV′) is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

What is claimed is:

1. A compound having the structure of Formula (A): 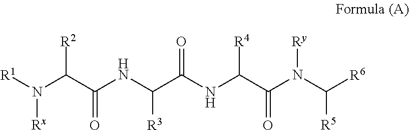
wherein:
R¹ is selected from: 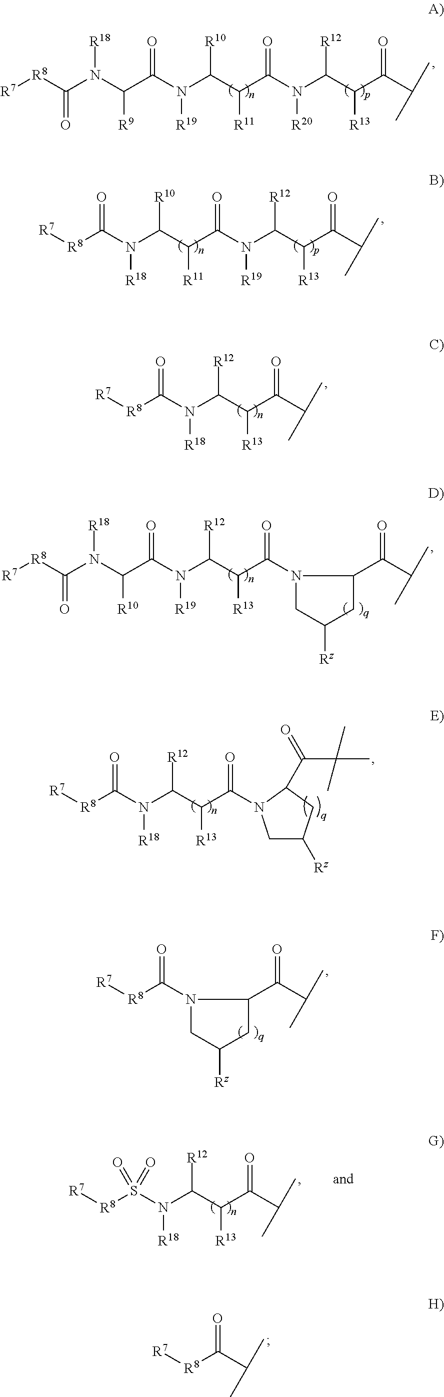
R², R⁴, R¹⁰, R¹¹, R¹², and R¹³ are each independently —H, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —CH₂—(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl, 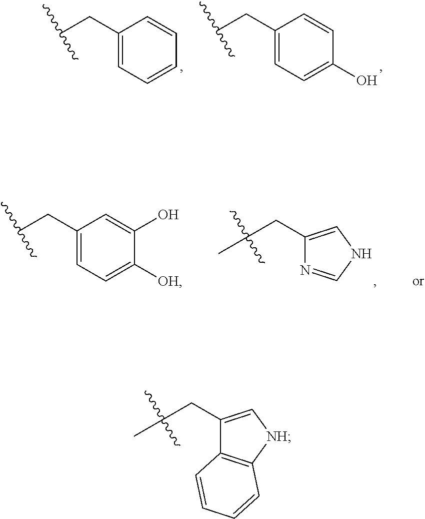
R³ is methyl, ethyl, isopropyl, or cyclopropyl;
R⁵ is H, methyl, ethyl, or —CH₂OH;
R⁶ is —C(═O)R¹⁴;
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is optionally substituted aryl, optionally substituted heterocycloalkyl, optionally substituted alkenyl, or a linear or branched alkyl chain of about 1-22 carbon atoms, optionally comprising within the alkyl chain or at an alkyl chain terminus an optionally substituted aryl, an optionally substituted heterocycloalkyl, or an optionally substituted 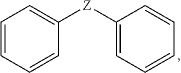
wherein Z is a bond, O, S, NH, CH₂, NHCH₂, or C≡C;
R⁸ is a bond, —O—, or —N(R¹⁷)—, optionally substituted C₁-C₆alkyl, optionally substituted C₁-C₆heteroalkyl, optionally substituted heterocycloalkyl, optionally substituted aryl, or optionally substituted heteroaryl;
R⁹ is —CH₂OH, —CH₂CH(CH₃)₂, 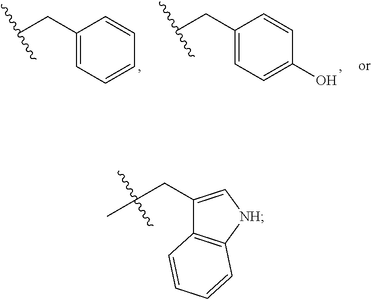
R¹⁴ is C₁-C₆alkyl, C₁-C₆haloalkyl, —C(O)OR²⁸, —CF₂C(O)OH, or 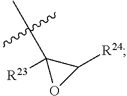
R¹⁵ and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ is H, C₁-C₄alkyl, or C₁-C₄alkoxy;
R²⁴ is —H, —CH₂OH, —CH(OH)(CH₃), —CH₂CF₃, —C(O)R²⁶, —C(O)OR²⁶, —C(O)NR²⁶R²⁷, CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl, 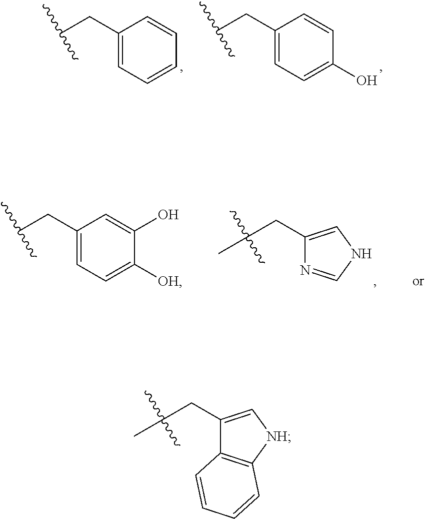
each R²⁵ is independently C₁-C₆alkyl;
R²⁶ is H, or C₁-C₄alkyl;
R²⁷ is H, or C₁-C₄alkyl;
R²⁸ is C₁-C₆alkyl;
n is 0 or 1;
p is 0 or 1; and
q is 0 or 1;
or a pharmaceutically acceptable salt, solvate, or prodrug thereof.

2. The compound of claim 1 wherein R¹ is

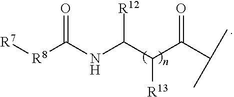

3. The compound of claim 1 wherein R¹ is

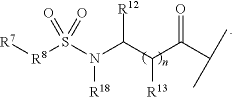

4. The compound of any one of claims 1-3 wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

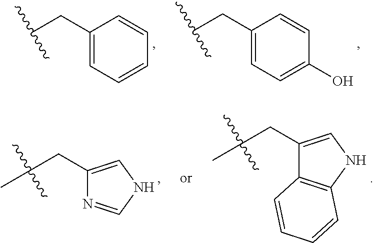

5. The compound of any one of claims 1-4 wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

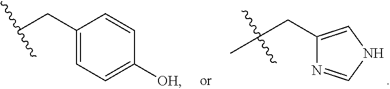 [SMILES: *Cc1ccc(O)cc1 ; *Cc1c[nH]cn1]

6. The compound of any one of claims 1-5 wherein R⁷ is a linear or branched alkyl chain of 1-22 carbon atoms, optionally comprising within the alkyl chain or at an alkyl chain terminus an optionally substituted aryl, an optionally substituted heteroaryl, an optionally substituted heterocycloalkyl, or an optionally substituted 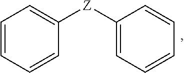
wherein Z is a bond, O, S, NH, CH₂, NHCH₂, or C≡C.

7. The compound of any one of claims 1-6 wherein R⁷ is a linear or branched alkyl chain of 1-22 carbon atoms, optionally comprising within the alkyl chain or at an alkyl chain terminus an optionally substituted 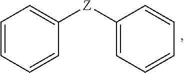
wherein Z is a bond.

8. The compound of any one of claims 1-7 wherein R⁷ is

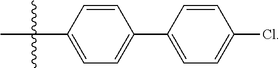 [SMILES: *c1ccc(-c2ccc(Cl)cc2)cc1]

9. The compound of any one of claims 1-8 wherein R⁸ is optionally substituted C₁-C₆heteroalkyl.

10. The compound of any one of claims 1-8 wherein R⁸ is a bond.

11. The compound of any one of claims 1-10 wherein R¹⁴ is —C(O)OR²⁸.

12. The compound of claim 11 wherein R²⁸ is CH₃.

13. The compound of any one of claims 1-10 wherein R¹⁴ is

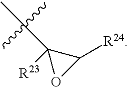

14. The compound of claim 13 wherein R²³ is H or C₁-C₄alkyl; and R²⁴ is H or optionally substituted C₁-C₈alkyl.

15. A compound having the structure of Formula (XIV): 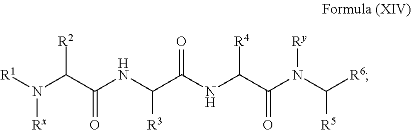
wherein:
R¹ is selected from: 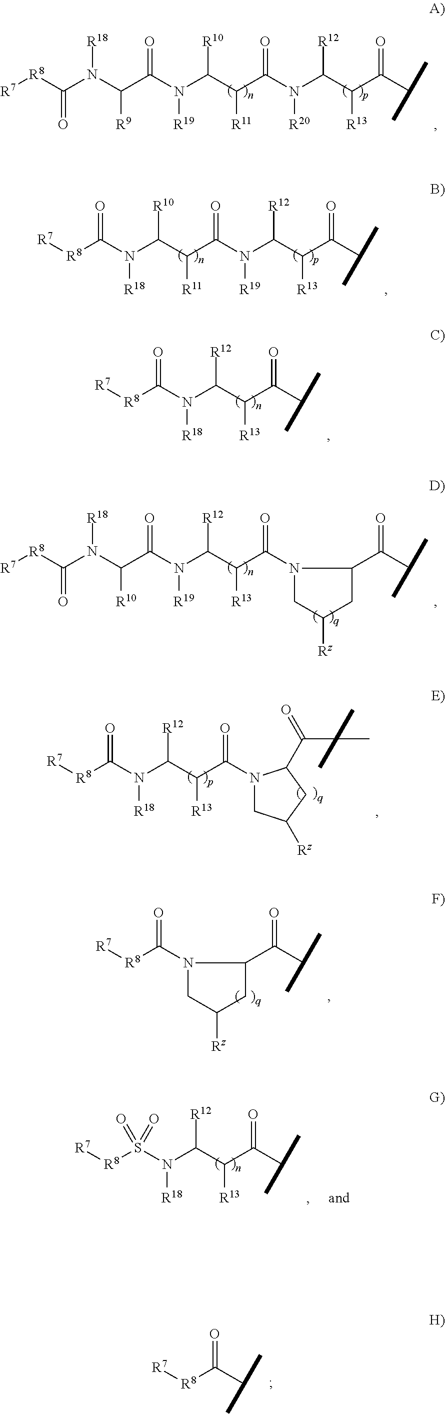
R², R⁴, R¹⁰, R¹¹, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂C(O)OR²⁵, —CH₂CH₂C(O)OH, —CH₂CH₂C(O)OR²⁵, —CH₂C(O)NH₂, —CH₂CH₂C(O)NH₂, —CH₂CH₂C(O)N(H)C(H)(CH₃)CO₂H, —CH₂CH₂C(O)N(H)C(H)(CO₂H)CH₂CO₂H, —CH₂NR²¹R²², —(CH₂)₂NR²¹R²², —(CH₂)₃NR²¹R²², —(CH₂)₄NR²¹R²², —(CH₂)₄N⁺(R²⁵)₃, —(CH₂)₄N(H)C(O)(2,3-dihydroxybenzene), optionally substituted C₁-C₈alkyl, optionally substituted C₁-C₈heteroalkyl, optionally substituted C₃-C₈cycloalkyl, optionally substituted —CH₂—C₃-C₈cycloalkyl, optionally substituted heterocycloalkyl, optionally substituted aryl, optionally substituted heteroaryl, 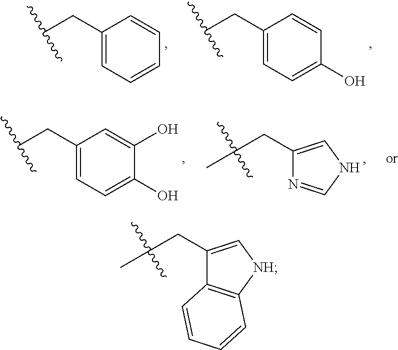
R³ is methyl, ethyl, isopropyl, or cyclopropyl;
R⁵ is H, methyl, ethyl, or —CH₂OH; or R⁵ and R²⁴ together with the boron atom form a 5- or 6-membered boron containing ring;
R⁶ is —C(═O)H, —CH₂C(═O)H, —C(═O)NHCH₂C(═O)H, —C(═O)C(═O)N(R¹⁴)₂, —C(═O)C(═O)OH, —B(OR²³)(OR²⁴), or 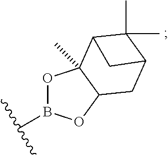 [SMILES: *B1OC2CC3CC(C3(C)C)[C@]2(C)O1]
or R⁵ and R⁶ together with the carbon atom form 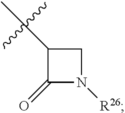
R^{x} is H, optionally substituted C₁-C₆alkyl, optionally substituted C₁-C₆heteroalkyl, or optionally substituted C₃-C₈cycloalkyl; or R^{x} and R² together with the nitrogen atom form an optionally substituted nitrogen containing ring;
R^{y} is H or methyl; or R^{y} and R⁵ together with the nitrogen atom form an optionally substituted nitrogen containing ring;
R^{z} is —NR¹⁵R¹⁶, —CH₂—NR¹⁵R¹⁶, or —(CH₂)₂—NR¹⁵R¹⁶;
R⁷ is unsubstituted C₁-C₁₀alkyl;
R⁸ is optionally substituted C₁-C₁₀heteroalkyl;
R⁹ is —CH₂OH, —CH₂CH(CH₃)₂, 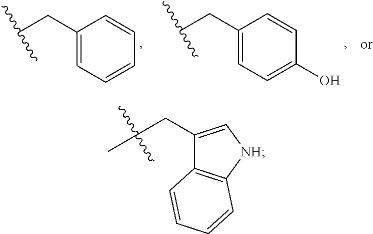
R¹⁴, R¹⁵, and R¹⁶ are each independently H, or C₁-C₄alkyl;
R¹⁷ is H, methyl, ethyl, isopropyl, or cyclopropyl;
R¹⁸, R¹⁹, and R²⁰ are each independently H, or methyl;
each R²¹ is independently H, or C₁-C₄alkyl;
each R²² is independently H, C₁-C₄alkyl, —C(═NH)(NH₂), or —CH(═NH);
R²³ and R²⁴ are each independently H, or C₁-C₄alkyl; or R²³ and R²⁴ together with the boron atom form an optionally substituted 5- or 6-membered boron containing ring;
each R²⁵ is independently C₁-C₆alkyl;
R²⁶ is H, C₁-C₄alkyl, C₁-C₄alkoxy, —CH₂C(O)OR²⁵, or —OCH₂C(O)OR²⁵;
n is 0 or 1;
p is 0 or 1; and
q is 0 or 1;
or a pharmaceutically acceptable salt, solvate, or prodrug thereof.

16. The compound of claim 15 wherein R¹ is

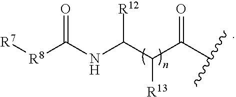

17. The compound of claim 15 wherein R¹ is

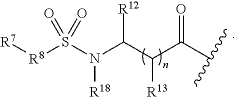

18. The compound of any one of claims 15-17 wherein R², R⁴, R¹², and R¹³ are each independently —H, —CH₃, —CH(CH₃)₂, —C(CH₃)₃, —CH(CH₃)(CH₂CH₃), —CH₂CH(CH₃)₂, —CH₂OH, —CH(OH)(CH₃), —CH₂CF₃, —CH₂C(O)OH, —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

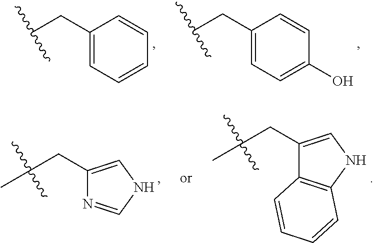

19. The compound of any one of claims 15-18 wherein R², R⁴, R¹², and R¹³ are each independently —H, —CH₃, —CH₂CH(CH₃)₂, —CH₂OH, —CH(OH)(CH₃), —CH₂CH₂C(O)OH, —CH₂C(O)NH₂, —CH₂CH₂C(O)NH₂, —CH₂NH₂, —(CH₂)₂NH₂, —(CH₂)₃NH₂, —(CH₂)₄NH₂,

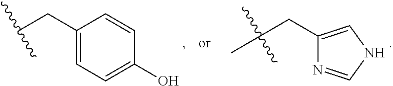 [SMILES: *Cc1ccc(O)cc1 ; *Cc1c[nH]cn1]

20. The compound of any one of claims 15-19 wherein R⁷ is unsubstituted C₁-C₈alkyl.

21. The compound of any one of claims 15-20 wherein R⁸ is a substituted C₁-C₈heteroalkyl.

22. The compound of any one of claims 15-20 wherein R⁸ is an unsubstituted C₁-C₈heteroalkyl.

23. The compound of any one of claims 15-22 wherein R⁶ is

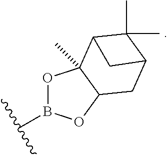 [SMILES: *B1OC2CC3CC(C3(C)C)[C@]2(C)O1]

24. The compound of any one of claims 15-22 wherein R⁶ is —B(OH)₂.

25. The compound of any one of claims 15-22 wherein R⁶ is —C(═O)C(═O)OH.

26. The compound of any one of claims 1-25 wherein n is 0.

27. A pharmaceutical composition comprising a compound of claim 1 or claim 15 and a pharmaceutically acceptable excipient.

28. A method of treatment of a bacterial infection in a mammal, comprising administering an effective amount of a compound of claim 1 or claim 15 to the mammal at a frequency and for a duration sufficient to provide a beneficial effect to the mammal.

29. The method of claim 28, wherein the bacterial infection is an infection involving Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

30. The method of claim 28 further comprising administering a second therapeutic agent.
